SEQUENCE LISTING (2 sequences)

SEQ ID NO: 1 - protein, 188 aa, Homo sapiens [sequence omitted]

SEQ ID NO: 2 - protein, 1333 aa, Homo sapiens [sequence omitted]

(19) United States
(12) Patent Application Publication
(10) Pub. No.: US 2026/0294925 A1
(43) Pub. Date: Oct. 1, 2026

(54) CELL PROLIFERATION MODULATORS AND USES THEREOF

(71) Applicant: Kumquat Biosciences Inc., San Diego, CA (US)

(72) Inventors: Yi Liu, San Diego, CA (US); Anne Uyei, Palo Alto, CA (US); Matthew R. Janes, Carlsbad, CA (US); Jeffrey Chen, San Diego, CA (US)

(21) Appl. No.: 19/548,258

(22) Filed: Feb. 24, 2026

Related U.S. Application Data
(63) Continuation of application No. PCT/US2024/045997, filed on Sep. 10, 2024.
(60) Provisional application No. 63/581,969, filed on Sep. 11, 2023.
(60) Provisional application No. 63/572,066, filed on Mar. 29, 2024.

(51) Int. Cl. A61K 31/541; A61K 31/4439; A61K 31/496; A61K 31/5025; A61K 31/504; A61K 31/506; A61K 31/519; A61K 31/529; A61K 31/5377; A61P 35/02

(52) U.S. Cl. CPC A61K 31/541; A61K 31/4439; A61K 31/496; A61K 31/5025; A61K 31/504; A61K 31/506; A61K 31/519; A61K 31/529; A61K 31/5377; A61P 35/02

(57) ABSTRACT

The present disclosure provides compounds and pharmaceutically acceptable salt thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of International Application No. PCT/US2024/045997, filed Sep. 10, 2024, which claims the benefit of U.S. Provisional Application No. 63/581,969, filed Sep. 11, 2023, and U.S. Provisional Application No. 63/572,066, filed Mar. 29, 2024, each incorporated herein by reference in its entirety.

### SEQUENCE LISTING

The instant application contains a Sequence Listing which has been submitted electronically in XML format and is hereby incorporated by reference in its entirety. Said XML copy, created on Feb. 24, 2026, is named 56690_772_301_SL.xml and is 4,104 bytes in size.

### BACKGROUND

The Philadelphia positive chromosome (Ph+) abnormality resulting from reciprocal translocation of t(9; 22) (q34; q11) is associated with several types of blood cancer. This translocation of the long arms of chromosomes 9 and 22 transposes the c-ABL1 gene from chromosome 9 to the 5′ half of the BCR gene on chromosome 22, creating a new hybrid gene called BCR-ABL, coding for p210^{BCR-ABL} or p190^{BCR-ABL}, depending on the location of the breakpoint on chromosome 22. This cytogenetic hallmark is present in over 90% of patients with chronic myelogenous leukemia (CML), in 20% to 35% of adults with acute lymphocytic leukemia (ALL) and in 5% or fewer children with ALL or patients with acute myelogenous leukemia. In 2023, an estimate of 9,000 new people will be diagnosed with CML and approximately 1,310 people will die of CML in the United States. In addition, about 6,540 new cases of ALL (20-35% of which will be Ph+) will be diagnosed and about 1,390 deaths will result from ALL.

The advent of generations of tyrosine kinase inhibitors, starting with imatinib as the first generation, have remarkably improved the life expectancy of patients. The majority of these tyrosine kinase inhibitors target the ATP site of the BCR-ABL oncoprotein, with an exception of asciminib that binds to an allosteric site. However, chronic use of the tyrosine kinase inhibitors has caused drug resistance with an alarming prevalence in 20-30% of treated CML patients. Am. J. Hematol. 2016; 91:252-265; Blood. 2012; 119:1123-1129. Over 90 different mutations in BCR-ABL have been detected. Amongst them, point mutations T315, Y253, E255, M351, G250, F359, and H396 are most common and correlated with drug resistance. JCO Glob. Oncol. 2021; 7: GO.21.00058.

Son of Sevenless 1 (SOS1), a homolog of SOS2 in mammalian cells, acts as a guanine nucleotide exchange factor (GEF) that promotes release of inactive GDP from RAS-family proteins to enable GTP binding (Chardin et al., Science, 1993, 260(5112):1338-43). SOS1 has been implicated in cancer via its ability to activate RAS-family protein signaling. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, TrkA, TrkB, TrkC, RET, c-MET, VEGFR1/2/3). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275(8):5966-75). This localization of SOS1 to the plasma membrane, proximal to RAS-family proteins, enables SOS1 to promote RAS-family protein activation. SOS1-activation of RAS-family proteins can also be mediated by the interaction of SOS1/Grb2 with the BCR-ABL oncoprotein commonly found in chronic myelogenous leukemia (Kardinal et al., 2001, Blood, 98:1773-81).

### SUMMARY

There exists a considerable need for identifying relevant monotherapy as well as combination therapy to address the long-felt need for gaining sustained therapeutic benefit against Philadelphia positive chromosome (Ph+) diseases, including various types of blood cancer. The present invention addresses these needs and provides related advantages as well.

In an aspect, the present disclosure provides a method of treating a Philadelphia chromosome-positive (Ph+) blood cancer in a subject in need thereof, comprising administering a pharmaceutical composition comprising an effective amount of a small molecule SOS1 inhibitor to the subject, wherein said subject has been treated with a tyrosine kinase inhibitor against BCR-ABL tyrosine kinase (TKI) prior to said administration of the SOS1 inhibitor. Exemplary Ph+ diseases are various blood cancers including but not limited to chronic myelogenous leukemia (CML), Ph+ acute lymphoblastic leukemia (Ph+ALL), or Ph+ lymphoblastic lymphoma (Ph+LBL). Resistance to the TKI can be characterized by one or more selected from the group consisting of progression of the Philadelphia chromosome-positive (Ph+) blood cancer, mutation in BCR-ABL gene, overexpression of BCR-ABL gene, change in drug transporter activity, activation of compensatory signaling pathway, change in cell metabolism, epigenic alteration, change in microenvironment and immunological status, altered expression of microRNAs, alteration of DNA damage repair and genomic instability. Alternatively, resistance to the TKI can be characterized by one or more mutations in BCR-ABL gene resulting in a decrease of TKI binding affinity to BCR-ABL tyrosine kinase. In some embodiments, resistance to the TKI is characterized by a mutation (1) resulting in augmentation of BCR-ABL kinase activity, half-life of BCR-ABL in the cell, expression of BCR-ABL in the cell, or (2) a mutation in the P-loop (ATP-binding site), a mutation in C-loop (catalytic domain), a mutation on activation (A)-loop, a mutation in myristate pocket, and/or a mutation that directly affects the binding of the TKI (drug contact site). Non-limiting examples of mutation in BCR-ABL gene that is correlated with drug resistance include T315I, F359V, Y253H, E255K, M351T, G250E, F359I, and H396. In some embodiments, a subject exhibiting relapse of CML is characterized by renewed onset of an abnormal hematological response, molecular response, and/or cytogenetic response.

In another aspect, the present disclosure provides a method of treating Philadelphia chromosome-positive (Ph+) diseases, including blood cancers in a subject in need thereof, comprising administering a pharmaceutical composition comprising an effective amount of a small molecule SOS1 inhibitor, wherein the subject is identified as having a genetic aberration in BCR-ABL. In some embodiments, the genetic aberration is a mutation in BCR-ABL associated with resistance to a tyrosine kinase inhibitor against BCR-ABL tyrosine kinase (TKI). In some embodiments, the genetic aberration is characterized by a mutation in the P-loop (ATP-binding site), a mutation in C-loop (catalytic domain), a mutation on activation (A)-loop, a mutation in myristate pocket, and/or a mutation that directly affects the binding of the TKI (drug contact site). In some embodiments, the genetic aberration is characterized by a mutation in BCR-ABL gene selected from the group consisting of T315I, F359V, Y253H, E255K, M351T, G250E, F359I, and H396.

In another aspect, the present disclosure provides a method of combination therapy, comprising administering (a) a small molecule SOS1 inhibitor and (b) a tyrosine kinase inhibitor against BCR-ABL tyrosine kinase (TKI) to a subject in need thereof, wherein the administering of (a) takes places prior to, concurrent with, or subsequent to administering (b), and wherein the combination therapy synergistically reduces progression of a Philadelphia chromosome-positive (Ph+) blood cancer, and/or reduces undesirable side effect associated with the TKI. In some embodiments, one or both of (a) and (b) are administered at a sub-therapeutic dose but achieving a therapeutic effect at least comparable to administering (a) or (b) alone when (a) or (b) is administered at the respective therapeutically effective amount. In some embodiments, the combination therapy exhibits a synergistic effect in reducing progression of chronic myelogenous leukemia (CML), Ph+ acute lymphoblastic leukemia (Ph+ALL), or Ph+ lymphoblastic lymphoma (Ph+LBL). In some embodiments, reducing progression of a Philadelphia chromosome-positive (Ph+) blood cancer is evidenced by an improvement of a response selected from the group consisting of hematologic response, molecular response, and cytogenetic response of said subject. In some embodiments, an improvement of hematologic response is characterized by blood cells of the subject return to normal levels. In some embodiments, an improvement of molecular response is characterized by a reduction in amount of BCR-ALB1 gene present in the subject's blood as ascertained by a nucleic acid assay. In some embodiments, an improvement of cytogenetic response is characterized by a reduction in number of bone marrow cells comprising Philadelphia chromosome. In some embodiments, the combination therapy exhibits a synergistic effect in reducing side effects associated with the TKI, including but not limited to cardio-toxicity, nausea, vomiting, diarrhea, muscle cramp, bone pain, fatigue, rashes, and edema.

Where desired, the TKI used in the combination therapy is dasatinib administered at a sub-therapeutic dose of less than 100 mg daily for treating chronic phase CML or less than 140 mg daily for treating accelerated and blast phase CML and Ph+ ALL. In some embodiments, the TKI used in the combination therapy is imatinib administered at a sub-therapeutic dose of less than 400 mg daily for treating chronic phase CML or less than 600 mg daily for treating accelerated and blast phase CML and Ph+ ALL. In some embodiments, the TKI used in the combination therapy is nilotinib administered at a sub-therapeutic dose of less than 300 mg twice daily for chronic phase CML or less than 400 mg twice a day for resistant (second line) and accelerated or blast phase of CML. In some embodiments, the TKI used in the combination therapy is asciminib administered at a sub-therapeutic dose of less than 80 mg daily or less than 40 mg twice daily, or less than 200 mg twice a day for Ph+ blood cancer having T315I mutation. In some embodiments, the TKI used in the combination therapy is ponatinib administered at a sub-therapeutic dose of less than 45 mg daily. In some embodiments, the TKI used in the combination therapy is radotinib, flumatinib, or olverembatinib administered at a sub-therapeutic dose approved for its intended indication.

In another aspect, the present disclosure provides a method of treating Philadelphia chromosome-positive (Ph+) blood cancer in a subject in need thereof. The method may comprise administering a pharmaceutical composition that comprises an effective amount of a small molecule SOS1 inhibitor to the subject, wherein said SOS1 inhibitor inhibits growth of a CML cell line with an IC50 less than about 50 nM as ascertained in a growth inhibition assay utilizing the CML cell line.

In yet another aspect, the present invention provides a method of reducing proliferation of a cell comprising a Philadelphia chromosome. The method comprises administering to the cells (a) a small molecule SOS1 inhibitor disclosed herein, and (b) at least one tyrosine kinase inhibitor against BCR-ABL tyrosine kinase (TKI), wherein the administration of (a) and (b) synergistically inhibits growth of CML cells as evidenced by achieving a comparable or higher degree of growth inhibition when either (1) less than 90% of the SOS1 inhibitor is administered as compared to the amount required for the SOS1 inhibitor when administered alone; or (2) less than 90% of the TKI is administered as compared to the amount required for TKI when administered alone. In some embodiments, the TKI administered is less than about 80%, 70%, 60%, 50%, 40%, 30%, 25%, 20%, 15%, or 10% of the amount required to achieve a comparable or higher degree of growth inhibition. In some embodiments, the practice of this method of reducing proliferation of Ph+ chronic myelogenous leukemia (CML) cells, Ph+ALL cells, or Ph+LBL is in vitro, ex vivo, or in vivo.

In yet another aspect, the present disclosure provides a method of reducing proliferation of a cell comprising a Philadelphia chromosome, the method comprising administering to the cells (a) a small molecule SOS1 inhibitor and (b) at least one tyrosine kinase inhibitor against BCR-ABL (TKI), wherein the administration of (a) and (b) synergistically inhibits growth of CML cells with a synergistic value of at least about 0.1, 0.2, 0.3, 0.4, 0.5, 1, 1.5, 2, 2.5, 3, 3.5, 4, 4.5, 5, 5.5, 6, 6.5, 7, 7.5, 8, 9, 10, 11, 12, 13, 14, 15, or above as ascertained by the Bliss independent criterion. In some embodiments, a subject combination treatment can be characterized by a synergistic value of about 0.1 to about 1, about 1.0-5, about 5 to about 10, or about 10-15 as ascertained by Bliss independent criterion. The synergistic value is ascertained by Bliss independent criterion in accordance to the formula:

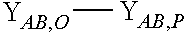

wherein:
Y_{AB,O} is observed percentage growth inhibition of the cancer cells by the application of (a) and (b) comprising (a) at dose A and (b) at dose B; and
Y_{AB,P} is predicted percentage growth inhibition of the cancer cells by the application of (a) and (b) comprising (a) at the dose A, and (b) at the dose B, wherein Y_{AB,P}=Y_{A}+Y_{B}−Y_{A}Y_{B},
wherein further:
Y_{A} is observed percentage growth inhibition of the cancer cells by (a) alone at the dose A;
Y_{B} is observed percentage growth inhibition of the cancer cells by (b) alone at the dose B; and
Y_{A}Y_{B} is product of Y_{A} and Y_{B}.

Any methods disclosed herein may be applied to treat Ph+ blood cancers including chronic myelogenous leukemia (CML), Ph+ acute lymphoblastic leukemia (Ph+ALL), and Ph+ lymphoblastic lymphoma (Ph+LBL). A subject SOS1 inhibitor can be administered as a monotherapy, or in combination with an additional agent or additional therapy. A wide range of suitable additional agents include but are not limited to TKIs, immunomodulatory agents, anti-nausea agents (or an anti-emetic), pain reliever, and chemotherapeutic agents. In some embodiments, the additional agent is the same TKI that the subject has previously been administered. In some embodiments, additional agent is a TKI that is different from that administered previously to the subject. In some embodiments, the additional agent is the same TKI that the subject has previously been administered but to be administered at a dose different from a prior dose to the subject. In some embodiments, the additional agent is a different TKI administered with sub-therapeutic amount to the subject. Where desired, the additional agent can be an immunomodulatory agent, a cytokine or a checkpoint immune blockade agent. In some embodiments, the additional agent is selected from the group consisting of anti-PD-L1 antibody, anti-CTLA-4 antibody, anti-PD-1 antibody, anti-LAG3 antibody, anti-TIM3 antibody, and combinations thereof.

In some embodiments, the SOS1 inhibitor is administered with an additional therapy selected from the group consisting of surgery, cell therapy, chemotherapy, bone marrow transplant, or radiation.

Suitable TKIs for any of the methods disclosed herein are selected from the group consisting of imatinib, dasatinib, nilotinib, bosutinib, radotinib, flumatinib, ponatinib, olverembatinib and asciminib.

A SOS1 inhibitor for use in any of the methods disclosed herein typically exhibits a high degree of potency against SOS1 in a cell, and/or upon administration to a subject, results in downregulation of SOS1 in the subject. In some examples, the methods disclosed herein utilize a small molecule SOS1 inhibitor of Formula (I-1) or Formula (A-1).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a KCL22-xenograft study in which the mice were treated with or without a SOS1 inhibitor of the present disclosure (Compound A), alone or in combination with dasatinib or imatinib. The results demonstrate that a SOS1 inhibitor disclosed herein synergistically reduces tumor volume and extends duration of controlling tumor growth in combination with existing TKIs, and also induces sensitivity to imatinib resistance.

FIG. 2 shows a Ba/F3-p210-BCR-ABL1-T315I allograft study in which mice were treated with or without a SOS1 inhibitor of the present disclosure (Compound A), alone or in combination with asciminib or ponatinib.

FIG. 3A depicts percent growth inhibition of Ba/F3-p210-BCR-ABL1-T315I cells 5 days after treatment with a SOS1 inhibitor of the present disclosure (Compound A), ponatinib, or a 1:1 molar ratio of Compound A and ponatinib.

FIG. 3B depicts percent growth inhibition of Ba/F3-p210-BCR-ABL1-T315I cells 5 days after treatment with a SOS1 inhibitor of the present disclosure (Compound A), asciminib, or a 1:1 molar ratio of Compound A and asciminib.

FIG. 4 shows that a Ph+ pre-B-ALL cell line, SUP-B15, is sensitive to treatment with a SOS1 inhibitor of the present disclosure (Compound A) in a 3-day growth inhibition assay. The activity of Compound A is compared to other SOS1 inhibitors and to TKIs imatinib and dasatinib.

FIG. 5A shows a degree of growth inhibition of KCL-22 cells upon treatment with (i) a SOS1 inhibitor of the present disclosure (Compound A) alone, (ii) imatinib alone, or (iii) a combination of (i) and (ii) at various concentrations.

FIG. 5B shows a degree of synergy across the combinations in FIG. 5A, as calculated using the BLISS independent model.

FIG. 6A shows a degree of growth inhibition of KCL-22 cells upon treatment with (i) a SOS1 inhibitor of the present disclosure (Compound A) alone, (ii) dasatinib alone, or (iii) a combination of (i) and (ii) at various concentrations.

FIG. 6B shows a degree of synergy across the combinations in FIG. 6A, as calculated using the BLISS independent model.

FIG. 7 shows a KCL22-r xenograft study in which the mice were treated with or without a SOS1 inhibitor of the present disclosure (Compound A), alone or in combination with dasatinib. The results demonstrate that a SOS1 inhibitor disclosed herein synergistically reduces tumor volume in combination with dasatinib in a model that is refractory to dasatinib monotherapy.

FIG. 8 shows a KCL22-xenograft study in which mice with a heavy disease burden were treated with dasatinib, alone or in combination with a SOS1 inhibitor of the present disclosure (Compound A). The results demonstrate that a SOS1 inhibitor disclosed herein synergistically reduces tumor volume in combination with dasatinib in a model that mimics a remission induction type design.

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.); PCR 2: A Practical Approach (M. J. MacPherson, B. D. Hames and G. R Taylor eds. (1995)); Antibodies, A Laboratory Manual (Harlow and Lane, eds. (1988)); and Culture of Animal Cells: A Manual of Basic Technique and Specialized Applications, 6th Edition (R. I. Freshney, ed. (2010)).

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise.

“About” as used herein when referring to a measurable value such as an amount, a duration, and the like, is meant to encompass variations of ±10% of a stated number or value.

The term “C_{x-y}” or “Cₓ-C_{y}” when used in conjunction with a chemical moiety, such as alkyl, alkenyl, or alkynyl, is meant to include groups that contain from x to y carbons in the chain. For example, the term “C_{x-y} alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C₂₋₁₂ alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₁₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include C₃₋₁₀ monocyclic rings, C₆₋₁₂ bicyclic rings, C₆₋₁₂ spirocyclic rings, and C₆₋₁₂ bridged rings. Each ring of a bicyclic carbocycle may be selected from saturated, unsaturated, and aromatic rings. In some embodiments, the carbocycle is a C₆₋₁₂ aryl group, such as C₆₋₁₀ aryl. In some embodiments, the carbocycle is a C₆₋₁₂ cycloalkyl group. In some embodiments, the carbocycle is a C₆₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocycle. Exemplary carbocycles include cyclopentyl, cyclohexyl, cyclohexenyl, adamantly, phenyl, indanyl, and naphthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2 or 3 heteroatoms selected from O, S and N. Heterocycles include 3- to 10-membered monocyclic rings, 6- to 12-membered bicyclic rings, 6- to 12-membered spirocyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to a 5- to 12-membered aromatic ring that comprises at least one heteroatom, such as 1, 2 or 3 heteroatoms, selected from O, S and N. As used herein, the heteroaryl ring may be selected from monocyclic or bicyclic—including fused, spirocyclic and bridged ring systems—wherein at least one of the rings in the ring system is aromatic. The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line “ [symbol] ” drawn across a bond or a dashed bond “ [symbol] ” are used interchangeably herein to denote where a bond disconnection or attachment occurs. For example, in the structure

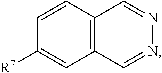

if R⁷ is 1-cyclopropyl-1-carbonitrile as in

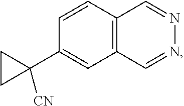 [SMILES: N#CC1(c2ccc3cnncc3c2)CC1]

then R⁷ may be depicted as

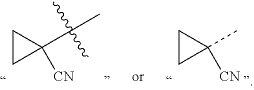

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I), is optionally substituted by one or more, such as 1, 2 or 3 substituents selected from:
halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²²)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from:
halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, and —N(R²²)(R²³);
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ¹⁸O, ¹⁷O, ³⁵S, ³⁶Cl, and ¹⁸F. Of particular interest are compound of Formula (I) enriched in tritium or carbon-14, which can be used, for example, in tissue distribution studies; compounds of the disclosure enriched in deuterium-especially at a site of metabolism-resulting, for example, in compounds having greater metabolic stability; and compounds of Formula (I) enriched in a positron emitting isotope, such as ¹¹C, ¹⁸F, ¹⁵O and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. In some embodiments, in order to optimize the therapeutic activity of the compounds of the disclosure, e.g., to treat fibrosis, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis-trans or E/Z isomers (geometric isomers), tautomeric forms and topoisomeric forms of the compounds described herein are included with the scope of the disclosure unless otherwise specified.

The term “pharmaceutically acceptable” refers to a material that is not biologically or otherwise unacceptable when used in the subject compositions and methods. For example, the term “pharmaceutically acceptable carrier” refers to a material—such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cancer” would include preventing cancer from occurring, ameliorating cancer, suppressing cancer, and alleviating the symptoms of cancer. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “administer”, “administering”, “administration”, and derivatives thereof refer to the methods that may be used to enable delivery of a composition to the desired site of biological action. These methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Administration is by any route, including parenteral. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease.

The term “effective amount” or “therapeutically effective amount” or “therapeutically effective dose” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The terms “sub-therapeutic amount”, “sub-effective amount”, and “sub-therapeutic dose” are used interchangeably and refer to the amount of an agent that is less than the effective amount for that agent, but when combined with an effective or sub-therapeutic amount of a different agent can produce a desired result, due to, for example, synergy in the resulting efficacious effects and/or reduced side effects by the combination of (i) the sub-therapeutic amount of the agent and (ii) the different agent (e.g., one or more different agents). For example, an agent can be approved for clinical use for a specified indication at a defined dose or range thereof (e.g., 150 milligrams per day (mg/d)) over the course of one or more administrations, and a sub-therapeutic amount of such agent can be lower than the approved dose or range thereof by at least about 0.1-fold, 0.2-fold, 0.5-fold, 1-fold, 2-fold, 5-fold, 10-fold, 20-fold, 50-fold, 100-fold, 200-fold, 500-fold, 1,000-fold, 2,000-fold, 5,000-fold, 10,000-fold, 20,000-fold, 50,000-fold, 100,000-fold, or more. The sub-therapeutic amount of such agent can be lower than the approved dose or range thereof by at most about 100,000-fold, 50,000-fold, 20,000-fold, 10,000-fold, 5,000-fold, 2,000-fold, 1,000-fold, 500-fold, 200-fold, 100-fold, 50-fold, 20-fold, 10-fold, 5-fold, 2-fold, 1-fold, 0.5-fold, 0.2-fold, 0.1-fold or less. A sub-therapeutic amount of an agent can be achieved by reducing the amount of the agent per dosage and/or by reducing the number of administrations (or cycles) of the agent to the subject.

The term “synergistic” or “synergizing” effect refers to when a desired effect (e.g., one or more different effects) of a combination (or combination treatment) comprising two or more different therapeutic components (e.g., two or more different therapies, two or more therapeutic agents, etc.) is greater than (i) the effect of each therapeutic component alone and/or (ii) the sum of the effect of each therapeutic component alone when administered individually (e.g., the sum of individual effects). The synergistic effect can be at least about 1%, 2%, 3%, 4%, 5%, 6%, 7%, 8%, 9%, 10%, 15%, 20%, 30%, 40%, 50%, 60%, 70%, 80%, 90%, 100%, 110%, 120%, 150%, 200%, 300%, 400%, 500%, 600%, 700%, 800%, 900%, 1,000%, 5,000%, or more than (i) the effect of each therapeutic component alone and/or (ii) the sum of individual effects. The effect can be any measurable effect including but not limited to an enhancement of a therapeutic effect of an individual component within the combination or a reduction in a side effect of an individual component within the combination. In some embodiments, the effect is a pharmacodynamic effect, such as phosphorylated ERK (p-ERK) and/or DUSP6 inhibition, optionally assessed in whole blood. To yield the synergistic effect, the two or more different therapeutic components of the combination treatment as disclosed herein can be administered concurrently or sequentially, as separate components or as a unit dosage. For example, a synergistic effect of a combination comprising a first agent and a second agent can yield a desired therapeutic outcome (e.g., in treating cancer) that is comparable (e.g., substantially the same) or better than (i) the therapeutic outcome of each therapeutic component alone at the therapeutically effective amount and/or (ii) the sum of individual effects, where either or both of the first and the second agent are administered in a respective sub-therapeutic amount. In another example, a synergistic effect of a combination comprising a first agent and a second agent can yield a desired therapeutic outcome (e.g., reducing side effect of either one of the agent) that is comparable (e.g., substantially the same) or better than the therapeutic outcome of each therapeutic component alone.

The term “IC50” refers to the half maximal inhibitory amount (e.g., concentration) of an inhibitor in inhibiting a biological or biochemical effect. IC50 can be a quantitative measure that indicates how much of a particular inhibitor is needed to inhibit a given biological or biochemical effect (e.g., expression and/or activity level of a gene/protein of interest, growth, or growth rate of a cell, etc.) by substantially half (e.g., about 50%). For example, determination of IC50 can be made by determining and constructing a dose-response curve and examining the effect of different concentrations of an inhibitor on reducing cell growth (e.g., inhibiting proliferation of cancer cells), and determining the concentration of the inhibitor at which 50% inhibition of cell growth is observed.

The term “combination”, as applied to agents including inhibitors disclosed herein, refers to the use of two or more agents (e.g., a SOS1 inhibitor and at least another inhibitor against a different signaling molecule) in vitro, in vivo, or ex-vivo. The two or more agents in combination can be formulated in one single formulation, or in separate formulation(s). A combination treatment or therapy with two or more agents can be carried out conjunctively in any temporal order, administered simultaneously or separately.

The term “conjunction” refers to a temporal aspect of the use of two or more agents (e.g., a SOS1 inhibitor and at least another inhibitor against a different signaling molecule) in vitro, in vivo, or ex-vivo. For example, one agent of a set of agents of interest can be administered prior to, subsequent to, or concurrently with the administration of a second agent of the set. Simultaneous administration can be effectuated by simultaneously administering multiple agents as separate agents, or as a unit dosage comprising the multiple agents.

The terms “antagonist” and “inhibitor” are used interchangeably, and they refer to a compound having the ability to inhibit a biological function (e.g., activity, expression, binding, protein-protein interaction) of a target protein (e.g., SOS1). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject”, “individual”, and “patient” are used interchangeably herein to refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like. Tissues, cells, and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

The term “small molecule” refers to one or more members from the group comprising an ion, a lipid, a chemical compound (e.g., natural or synthetic), and an amino acid (e.g., natural or synthetic), and a polypeptide (e.g., peptide or protein).

The term “nucleic acid agent” refers to an inhibitory agent capable of downregulating (e.g., reducing or inhibiting) expression and/or activity of a target moiety (e.g., a protein or a gene encoding thereof). In some embodiments, a nucleic acid agent may consist of a nucleic acid molecule. In some embodiments, a nucleic acid agent may comprise a nucleic acid molecule. In some embodiments, a nucleic acid agent may comprise a nucleic acid molecule and a non-nucleic acid molecule. The nucleic acid molecule and the non-nucleic acid molecule may be operatively coupled to each other to yield the inhibitory effect on the target moiety. The nucleic acid molecule and the non-nucleic acid molecule may be coupled (e.g., covalently and/or non-covalently) to each other. In some cases, the nucleic acid molecule and the non-nucleic acid molecule can be linked to each other via a linker. In some cases, the nucleic acid molecule can be configured to bind to the non-nucleic acid molecule. In some cases, the non-nucleic acid molecule can be configured to bind to the nucleic acid molecule. Non-limiting examples of the non-nucleic acid molecule include a small molecule, a polypeptide (e.g., an enzyme), etc. In some cases, the non-nucleic acid molecule is a nuclease, e.g., an endonuclease.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

An “antigen” is a moiety or molecule that contains an epitope, and, as such, also specifically binds to an antibody. An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, e.g., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

The term “diseased cell” refers to the state of a cell, tissue, or organism that diverges from the normal or healthy state. A diseased cell may result from the influence of a pathogen, a toxic substance, irradiation, or cell internal deregulation (e.g., genetic mutation). In an example, a diseased cell is a cell that has been infected with a pathogenic virus. In an example, a diseased cell is a malignant cell or neoplastic cell that may constitute or give rise to cancer in a subject (e.g., a mammal such as a human subject).

“Prodrug” is meant to indicate a compound that may be converted under physiological conditions or by solvolysis to a biologically active compound described herein (e.g., a compound of Formula (I)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (K-Ras or K-ras or Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

### Compounds

A SOS1 inhibitor for use in the present disclosure can be any SOS1 inhibitor that is known in the art, and can include any entity that, upon administration to a subject, results in downregulation of SOS1 in the subject. For example, a suitable SOS1 inhibitor can be selected from a variety of types of molecules. In particular, the SOS1 inhibitor can be a biological or chemical compound, such as a simple or complex organic or inorganic molecule, peptide, peptido mimetic, protein (e.g., antibody), liposome, or a polynucleotide (e.g., small interfering RNA, short hairpin RNA, microRNA, antisense, aptamer, ribozyme, triple helix). In some embodiments, a method disclosed herein utilizes a small molecule SOS1 inhibitor. As used herein, the term “small molecule” refers to a low molecular weight organic compound, such as a compound having a molecular weight of less than 1500 g/mol, less than 1250 g/mol, less than 1000 g/mol, or less than 750 g/mol. Many compounds are known to inhibit SOS1 (e.g., compounds of WO 2005/097119, which is incorporated herein by reference in its entirety).

In some embodiments, a small molecule SOS1 inhibitor may be conjugated to a degradation tag. A degradation tag may be configured to bind a degradation moiety having a capacity to degrade at least a portion of a target moiety that is bound by the degradation tag. In some embodiments, the target moiety is SOS1 or a substrate of SOS1.

In some embodiments, a subject SOS1 inhibitor disrupts the interaction between SOS1 and KRAS at an IC50 of less than about 20 nM, 10 nM, 5 nM, 4 nM, 3 nM, 2 nM, 1 nM, 0.5 nM, 0.4 nM, 0.3 nM, 0.2 nM, 0.1 nM or even less, as ascertained utilizing the Ras-SOS interaction assay described in Example 3. In some embodiments, the SOS1 inhibitor is at least 5-times more potent than BI-3406, MRTX0902, BAY 293, RMC-5845, or BI-1701963, such as at least 10-times, 20-times, 30-times, 40-times, 50-times, 60-times, 70-times, 80-times, 90-times, or 100-times more potent, as ascertained utilizing the Ras-SOS interaction assay described in Example 3. In some embodiments, a subject SOS1 inhibitor inhibits RAS signaling in a mutant receptor tyrosine kinase cell line, such as H1975, H358, or A375 cell lines, with an IC50 of less than about 10 nM, 5 nM, 4 nM, 3 nM, 2 nM, 1 nM, 0.5 nM, 0.4 nM, 0.3 nM, 0.2 nM, 0.1 nM or even less, as ascertained by a pERK inhibition assay. In some embodiments, the SOS1 inhibitor inhibits RAS signaling in a mutant receptor tyrosine kinase cell line, such as H1975, H358, or A375 cell lines, with at least 5-times more potency than BI-3406, MRTX0902, BAY 293, RMC-5845, or BI-1701963, such as at least 10-times, 50-times, 100-times, 200-times, 300-times, 500-times, 1000-times, 1500-times, or 2000-times more potency, as ascertained by a pERK inhibition assay. In some embodiments, a subject SOS1 inhibitor inhibits growth of a Ph+ cell, such as a CML cell line, with an IC50 less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, 0.1 nM or even less, as ascertained in a growth inhibition assay, optionally utilizing the CML cell line. In some embodiments, the SOS1 inhibitor inhibits a CML cell line selected from the group consisting of K562, BV173, KCL22-s, KCL22-imatinib-resistant cell line, Ba/F3 (BRC-ABL), Ba/F3 (BCR-ABL with T315I mutation), and Ba/F3 (BCR-ABL with F359V mutation) with an IC50 less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, 0.1 nM or even less. In some embodiments, the SOS1 inhibitor inhibits growth of a CML cell line with an IC50 at least 10, 50, 100, 200, 300, 500, or 1000 times less than that of

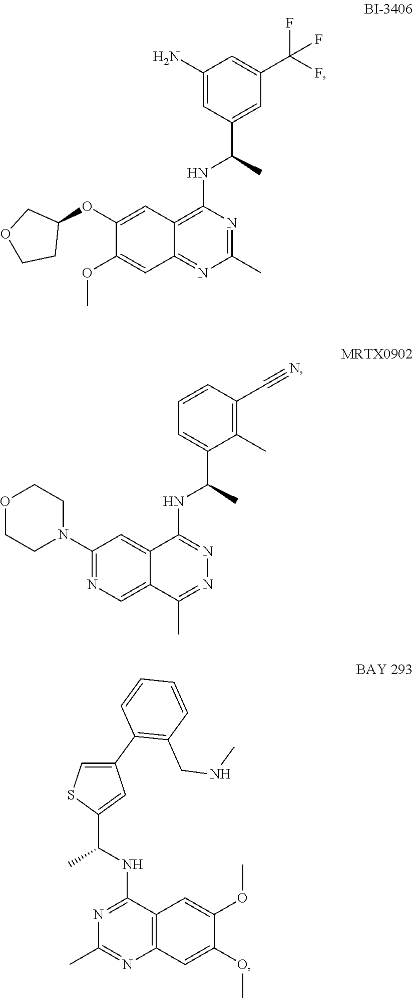

RMC-5845, or BI-1701963. In some embodiments, the SOS1 inhibitor inhibits growth of a CML cell line with an IC50 at least 10, 50, 100, 200, 300, 500, or 1000 times less than that of a SOS inhibitor described in WO2021092115, WO2018172250, WO2019201848, WO2019122129, WO2018115380, WO2021127429, WO2020180768, or WO2020180770, each of which is herein incorporated by reference in its entirety for all purposes. In some embodiments, the SOS1 inhibitor synergistically inhibits growth of a Ph+ cell, such as a CML cell line, in combination with a tyrosine kinase inhibitor against BCR-ABL tyrosine kinase (TKI). Exemplary TKI for use of such combination can be one or more of imatinib, dasatinib, nilotinib, bosutinib, radotinib, flumatinib, ponatinib, olverembatinib, and asciminib. In some embodiments, the SOS1 inhibitor is characterized in it synergistically inhibits growth of CML cells in combination with a TKI to yield at least about 80% growth inhibition when less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, or 0.1 nM of the SOS1 inhibitor is applied in combination with the TKI applied at its IC50 molarity, in an in vitro growth inhibition assay using CML cells. In some embodiments, the SOS1 inhibitor is characterized in that it synergistically increases percent of cell growth inhibition from about 10% to at least about 50%, when less than about 50 nM, 40 nM, 30 nM, 20 nM, 10 nM, 5 nM, 4 nM, 3 nM, 2 nM, or 1 nM of the SOS1 inhibitor is applied in combination with the TKI applied at its IC10 molarity, as ascertained in an in vitro growth inhibition assay using CML cells. In some embodiments, the SOS1 inhibitor is characterized in that it synergistically increases percent of cell growth inhibition from about 50% to at least about 80%, when less than about 50 nM, 40 nM, 30 nM, 20 nM, 10 nM, 5 nM, 4 nM, 3 nM, 2 nM, 1 nM of the SOS1 inhibitor is applied in combination with the TKI applied at its IC50 molarity, in an in vitro growth inhibition assay using CML cells.

The compounds of Formula (I) and (A) disclosed herein—including the compounds of Formula (I-A), (I-B), (I-1), (I-B1), (I-B2), (I-C), (I-C1), (I-C2), (I-C3), (I-D), (I-D1), (I-D2), (I-E), (I-E1), (II-B), (II-C), (III), (A-1), and (Aa-1)—or a pharmaceutically acceptable salt or solvate thereof, are SOS modulators and have a wide range of applications in therapeutics, diagnostics, and other biomedical research.

In certain aspects, the present disclosure provides a compound of Formula (I):

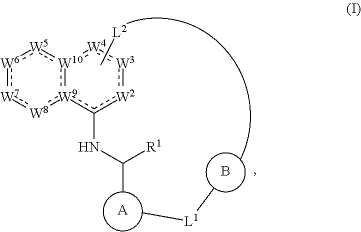

or a pharmaceutically acceptable salt or solvate thereof, wherein:

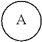

is selected from C₅₋₇ carbocycle and 5- to 7-membered heterocycle, each of which is optionally substituted with one or more R^{11a};

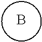

is absent or selected from C₃₋₈ carbocycle and 3- to 8-membered heterocycle, each of which is optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷;
L³ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b};
D is absent or selected from C₃₋₁₂ carbocycle and 3- to 12-membered heterocycle, each of which is optionally substituted with one or more R^{11d};
L⁴ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b};
W² is selected from N(R^{2b}), N, C(R²), C(R²)(R^{2a}), and C(O);
W³ is selected from N(R^{3b}), N, C(R³), C(R³)(R^{3a}), and C(O);
W⁴ is selected from N(R^{4b}), N, C(R⁴), C(R⁴)(R^{4a}), and C(O);
W⁵ is selected from N(R^{5b}), N, C(R⁵), C(R⁵)(R^{5a}), and C(O);
W⁶ is selected from N(R^{6b}), N, C(R⁶), C(R⁶)(R^{6a}), and C(O);
W⁷ is selected from N(R^{7b}), N, C(R⁷), C(R⁷)(R^{7a}), and C(O);
W⁸ is selected from N(R^{8b}), N, C(R⁸), C(R⁸)(R^{8a}), and C(O);
W⁹ is selected from N, C(R⁹), and C;
W¹⁰ is selected from N, C(R¹⁰), and C;
R¹ is C₁₋₃ alkyl optionally substituted with one or more R^{11c};
R², R^{2a}, R^{3a}, R^{4a}, R^{5a}, R^{6a}, R^{7a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{2b} and R^{8b} are each independently selected from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R^{3b}, R^{4b}, R^{5b}, R^{6b}, and R^{7b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R⁹ and R¹⁰ are each independently selected from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein each C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) is independently optionally substituted with one, two, or three R²⁰;
R¹¹, R^{11a}, and R^{11d} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11c} is independently selected at each occurrence from halogen, —OR¹², and —N(R¹²)(R¹³);
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In certain aspects, the present disclosure provides a compound of Formula (I):

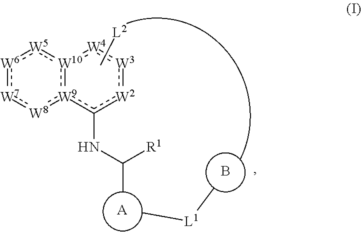

or a pharmaceutically acceptable salt or solvate thereof, wherein:

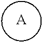

is selected from C₅₋₇ carbocycle and 5- to 7-membered heterocycle, each of which is optionally substituted with one or more R^{11a};

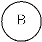

is absent or selected from C₃₋₈ carbocycle and 3- to 8-membered heterocycle, each of which is optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷;
W² is selected from N(R^{2b}), N, C(R²), C(R²)(R^{2a}), and C(O);
W³ is selected from N(R^{3b}), N, C(R³), C(R³)(R^{3a}), and C(O);
W⁴ is selected from N(R^{4b}), N, C(R⁴), C(R⁴)(R^{4a}), and C(O);
W⁵ is selected from N(R^{5b}), N, C(R⁵), C(R⁵)(R^{5a}), and C(O);
W⁶ is selected from N(R^{6b}), N, C(R⁶), C(R⁶)(R^{6a}), and C(O);
W⁷ is selected from N(R^{7b}), N, C(R⁷), C(R⁷)(R^{7a}), and C(O);
W⁸ is selected from N(R^{8b}), N, C(R⁸), C(R⁸)(R^{8a}), and C(O);
W⁹ is selected from N, C(R⁹), and C;
W¹⁰ is selected from N, C(R¹⁰), and C;
R¹ is C₁₋₃ alkyl optionally substituted with one or more R^{11c};
R², R^{2a}, R^{3a}, R^{4a}, R^{5a}, R^{6a}, R^{7a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{2b} and R^{8b} are each independently selected from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R^{3b}, R^{4b}, R^{5b}, R^{6b}, and R^{7b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R⁹ and R¹⁰ are each independently selected from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein each C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) is independently optionally substituted with one, two, or three R²⁰;
R¹¹ and R^{11a} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11c} is independently selected at each occurrence from halogen, —OR¹², and —N(R¹²)(R¹³);
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In some embodiments, the compound of Formula (I) is a compound of Formula (I-A):

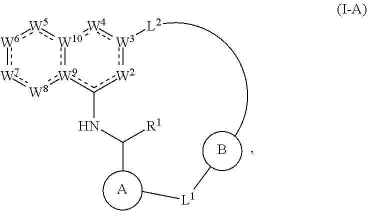

or a pharmaceutically acceptable salt or solvate thereof.

In some embodiments, the compound of Formula (I-A) is a compound selected from:

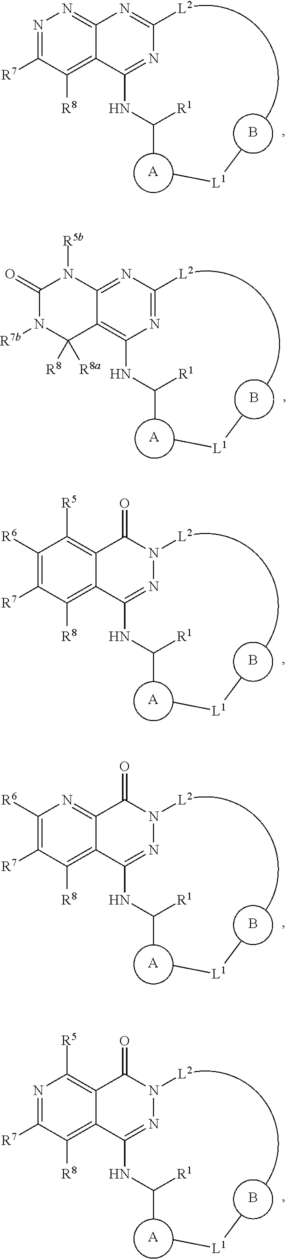

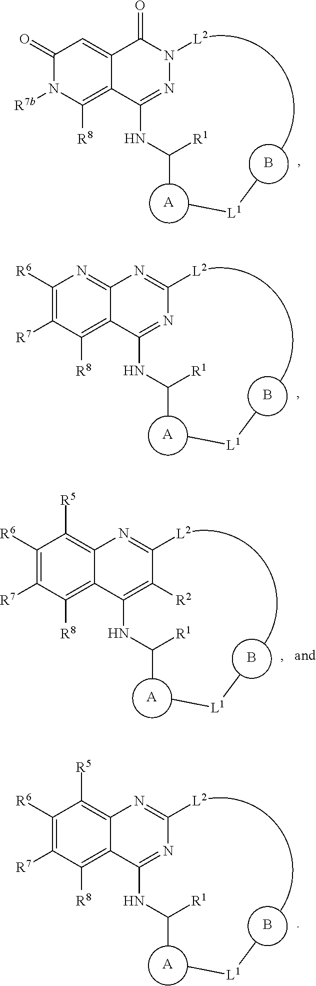

In some embodiments, the compound of Formula (I) is a compound of Formula (I-B), such as a compound of Formula (I-B1) or (I-B2):

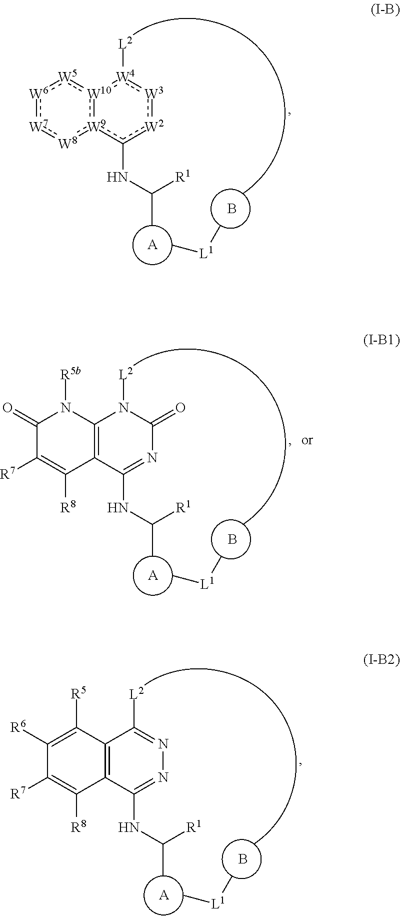

or a pharmaceutically acceptable salt or solvate thereof.

In some embodiments, the compound of Formula (I-B) is a compound selected from:

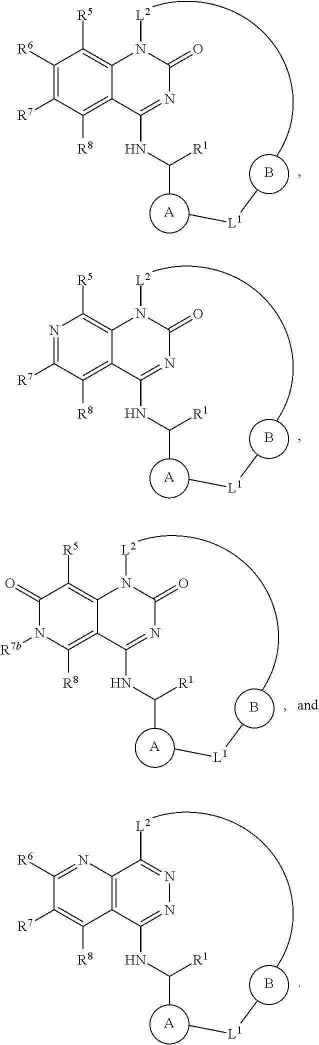

In some embodiments, the compound of Formula (I) is a compound of Formula (I-C), such as a compound of Formula (I-C1), (I-C2), or (I-C3):

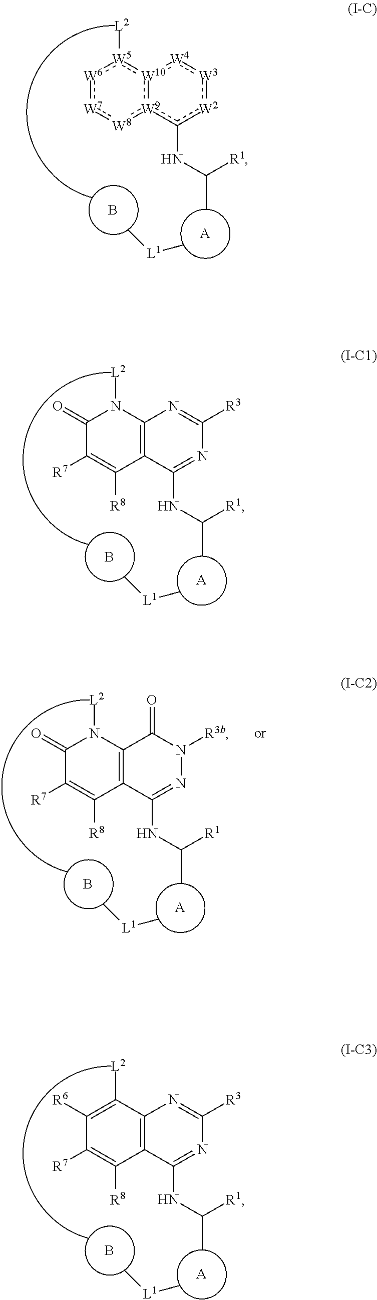

or a pharmaceutically acceptable salt or solvate thereof.

In some embodiments, the compound of Formula (I-C) is a compound selected from:

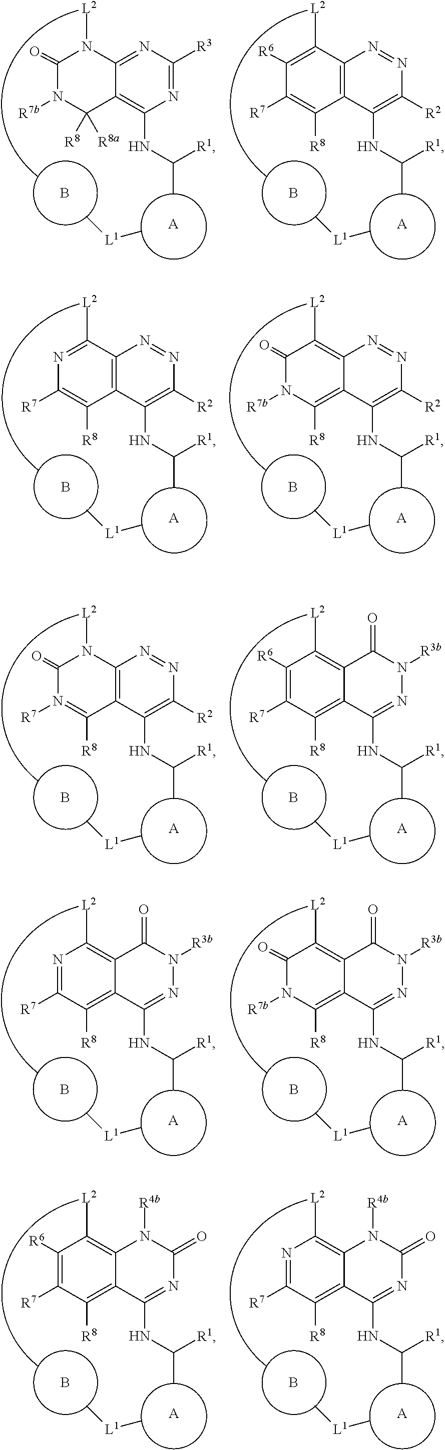

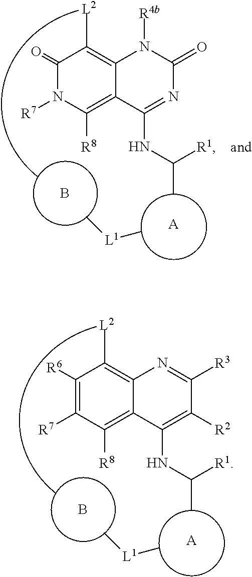

In some embodiments, the compound of Formula (I) is a compound of Formula (I-D), such as a compound of Formula (I-D1) or (I-D2):

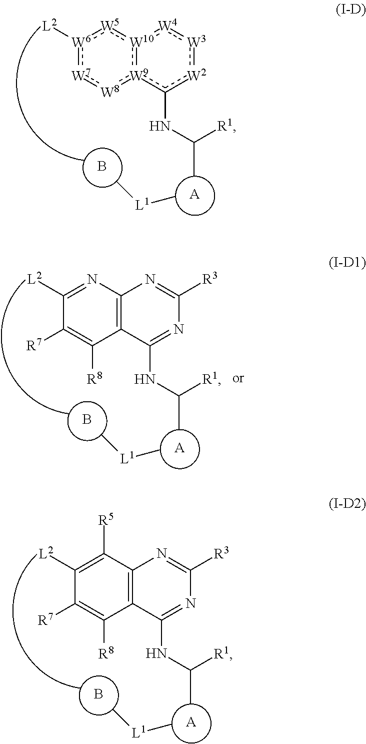

or a pharmaceutically acceptable salt or solvate thereof.

In some embodiments, the compound of Formula (I-D) is a compound selected from:

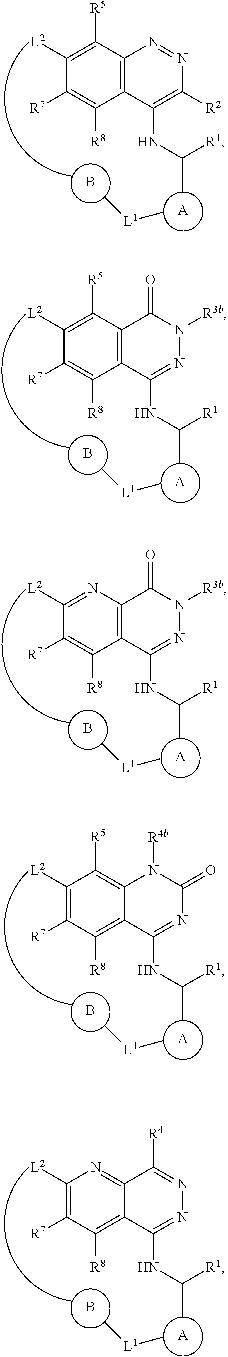

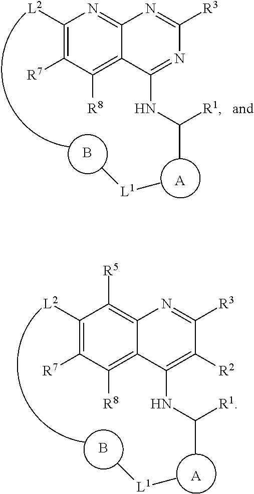

In some embodiments, the compound of Formula (I) is a compound of Formula (I-E), such as a compound of Formula (I-E1):

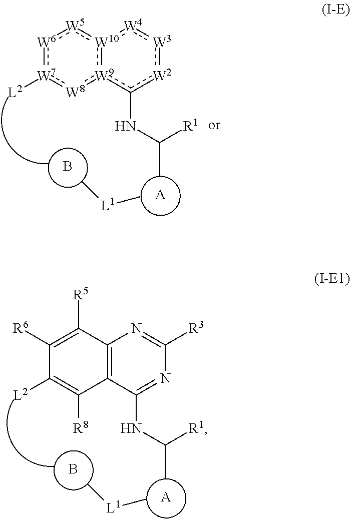

or a pharmaceutically acceptable salt or solvate thereof.

In some embodiments, the compound of Formula (I-E) is a compound selected from:

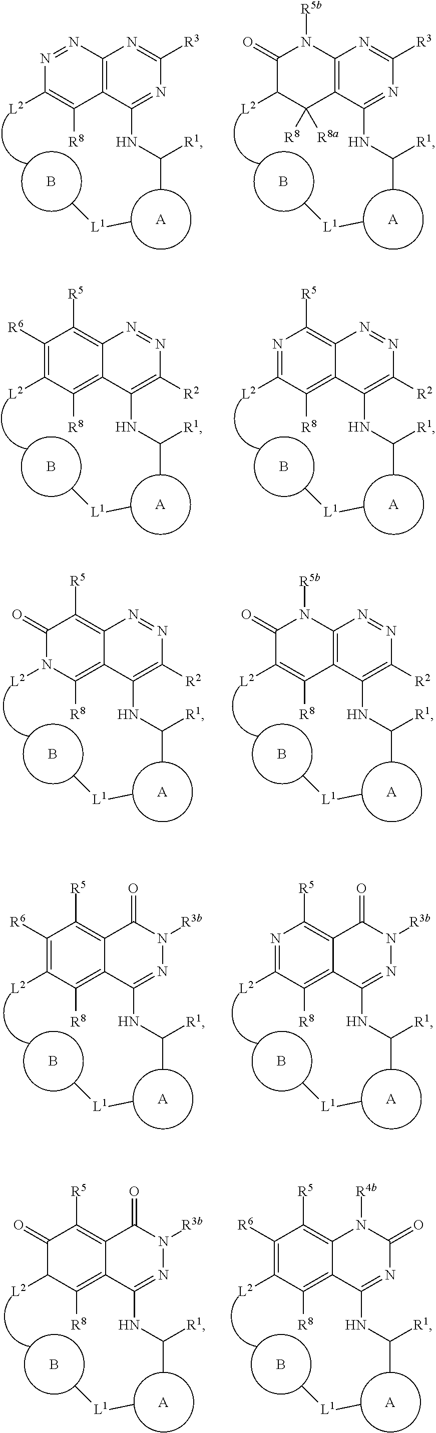

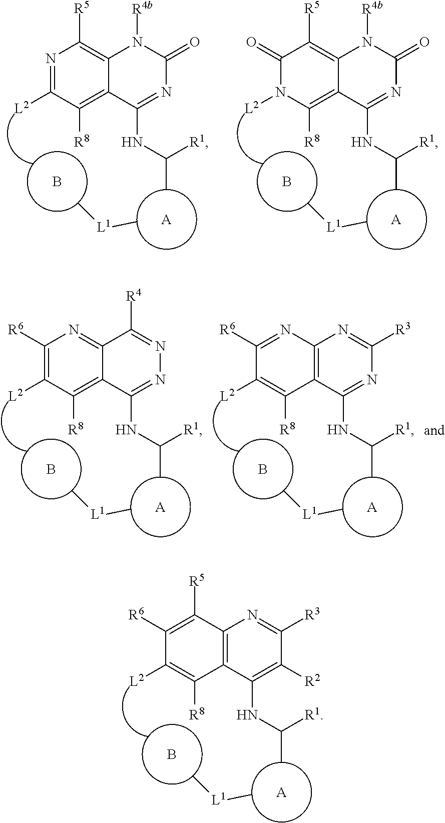

In some embodiments, for a compound of Formula (I), (I-A), (I-B), (I-C), (I-D), or (I-E), W² is N. In some embodiments, W³ is selected from N(R^{3b}), N, C(R³), and C(O), such as NCH₃, N, CH, CCH₃, and C(O). In some embodiments, W³ is selected from C(R³) and C(O), such as CH, CCH₃ and C(O). In some embodiments, W³ is CH. In some embodiments, W³ is CCH₃. In some embodiments, W⁴ is selected from N(R^{4b}), N, C(R⁴), and C(O), such as N(R^{4b}), N, C(R⁴), and C(O), wherein R^{4b} and R⁴ are each independently a bond to L². In some embodiments, W⁴ is selected from N(R^{4b}) and N, such as N(R^{4b}), wherein R^{4b} is a bond to L². In some embodiments, W⁴ is N. In some embodiments, W⁵ is selected from N(R^{5b}), N, C(R⁵), and C(O), such as N(R^{5b}), NCH₃, N, CH, C(R⁵), and C(O), wherein R^{5b} and R⁵ are each independently a bond to L². In some embodiments, W⁵ is selected from N(R^{5b}), N, and C(R⁵), such as N(R^{5b}), NCH₃, N, CH, and C(R⁵), wherein R^{5b} and R⁵ are each independently a bond to L². In some embodiments, W⁵ is selected from N(R^{5b}) and C(R⁵), such as N(R^{5b}), NCH₃, and CH, wherein R^{5b} is a bond to L². In some embodiments, W⁵ is N(R^{5b}). In some embodiments, W⁶ is selected from C(R⁶) and C(O), such as COCH₃, CH, C(R⁶), and C(O), wherein R⁶ is a bond to L². In some embodiments, W⁶ is C(O). In some embodiments, W⁷ is C(R⁷), such as W⁷ is C(R⁷) wherein R⁷ is a bond to L². In some embodiments, W⁷ is C(R⁷), wherein R⁷ is not hydrogen, such as R⁷ is selected from C₃₋₁₀ cycloalkyl, 3- to 10-membered heterocycloalkyl, and —OR¹², wherein C₃₋₁₀ cycloalkyl and 3- to 10-membered heterocycloalkyl are optionally substituted with one, two, or three R²⁰. In some embodiments, W⁸ is C(R⁸), such as W⁸ is CH. In some embodiments, W⁹ is C. In some embodiments, W¹⁰ is C.

In some embodiments, for a compound of Formula (I), (I-A), (I-C), (I-D), or (I-E), W² is N; W³ is N(R^{3b}); W⁴ is C(O); and W⁹ and W¹⁰ are each C, such as W² is N; W³ is NCH₃; W⁴ is C(O); and W⁹ and W¹⁰ are each C. In some embodiments, for a compound of Formula (I), (I-B), (I-C), (I-D), or (I-E), W² is N; W³ is C(O); W⁴ is N(R^{4b}); and W⁹ and W¹⁰ are each C, such as W² is N; W³ is C(O); W⁴ is N(R^{4b}), wherein R^{4b} is a bond to L²; and W⁹ and W¹⁰ are each C. In some embodiments, for a compound of Formula (I), (I-A), (I-C), (I-D), or (I-E), W² is N; W³ is C(R³); W⁴ is N; and W⁹ and W¹⁰ are each C, such as W² is N; W³ is CH or CCH₃; W⁴ is N; and W⁹ and W¹⁰ are each C. In some embodiments, for a compound of Formula (I), (I-A), (I-B), (I-C), (I-D), or (I-E), W⁵ is C(R⁵); W⁶ is C(R⁶); W⁷ is C(R⁷); W⁸ is C(R⁸); and W⁹ and W¹⁰ are each C, such as W⁵ is CH or C(R⁵), wherein R⁵ is a bond to L²; W⁶ is CH or C(R⁶), wherein R⁶ is a bond to L²; W⁷ is C(R⁷); W⁸ is CH; and W⁹ and W¹⁰ are each C. In some embodiments, for a compound of Formula (I), (I-A), (I-B), (I-C), or (I-E), W⁵ is N(R^{5b}); W⁶ is C(O); W⁷ is C(R⁷); W⁸ is C(R⁸); and W⁹ and W¹⁰ are each C, such as W⁵ is NCH₃ or N(R^{5b}), wherein R^{5b} is a bond to L²; W⁶ is C(O); W⁷ is C(R⁷); W⁸ is CH; and W⁹ and W¹⁰ are each C. In some embodiments, for a compound of Formula (I), (I-A), (I-B), (I-D), or (I-E), W⁵ is N; W⁶ is C(R⁶); W⁷ is C(R⁷); W⁸ is C(R⁸); and W⁹ and W¹⁰ are each C, such as W⁵ is N; W⁶ is COCH₃, CH, or C(R⁶), wherein R⁶ is a bond to L²; W⁷ is C(R⁷); W⁸ is CH; and W⁹ and W¹⁰ are each C. In some embodiments, for a compound of Formula (I), (I-A), (I-B), (I-C), (I-D), or (I-E), W² is N; W³ is selected from N(R^{3b}), N, C(R³), and C(O); W⁴ is selected from N(R^{4b}), N, C(R⁴), and C(O); W⁵ is selected from N(R^{5b}), N, and C(R⁵); W⁶ is selected from C(R⁶) and C(O); W⁷ is C(R⁷); W⁸ is C(R⁸); and W⁹ and W¹⁰ are each C, such as W² is N; W³ is selected from NCH₃, N, CH, CCH₃, and C(O); W⁴ is selected from N(R^{4b}), N, C(R⁴), and C(O), wherein R^{4b} and R⁴ are each independently a bond to L²; W⁵ is selected from N(R^{5b}), NCH₃, N, CH, and C(R⁵), wherein R^{5b} and R⁵ are each independently a bond to L²; W⁶ is selected from COCH₃, CH, C(R⁶), and C(O), wherein R⁶ is a bond to L²; W⁷ is C(R⁷); W⁸ is CH; and W⁹ and W¹⁰ are each C. In some embodiments, for a compound of Formula (I), (I-A), (I-B), (I-C), (I-D), or (I-E), W² is N; W³ is selected from C(R³) and C(O); W⁴ is selected from N(R^{4b}) and N; W⁵ is selected from N(R^{5b}) and C(R⁵); W⁶ is selected from C(R⁶) and C(O); W⁷ is C(R⁷); W⁸ is CH; and W⁹ and W¹⁰ are each C, such as W² is N; W³ is selected from CH, CCH₃, and C(O); W⁴ is selected from N(R^{4b}) and N, wherein R^{4b} is a bond to L²; W⁵ is selected from N(R^{5b}), NCH₃, CH, and C(R⁵), wherein R^{5b} and R⁵ are each independently a bond to L²; W⁶ is selected from COCH₃, CH, C(R⁶), and C(O), wherein R⁶ is a bond to L²; W⁷ is C(R⁷); W⁸ is CH; and W⁹ and W¹⁰ are each C.

In some embodiments, for a compound of Formula (I),

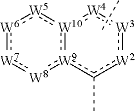

is selected from

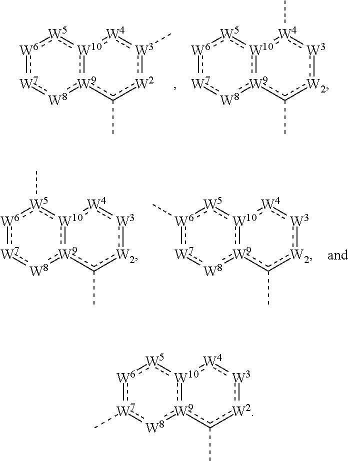

In some embodiments,

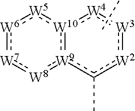

is selected from,

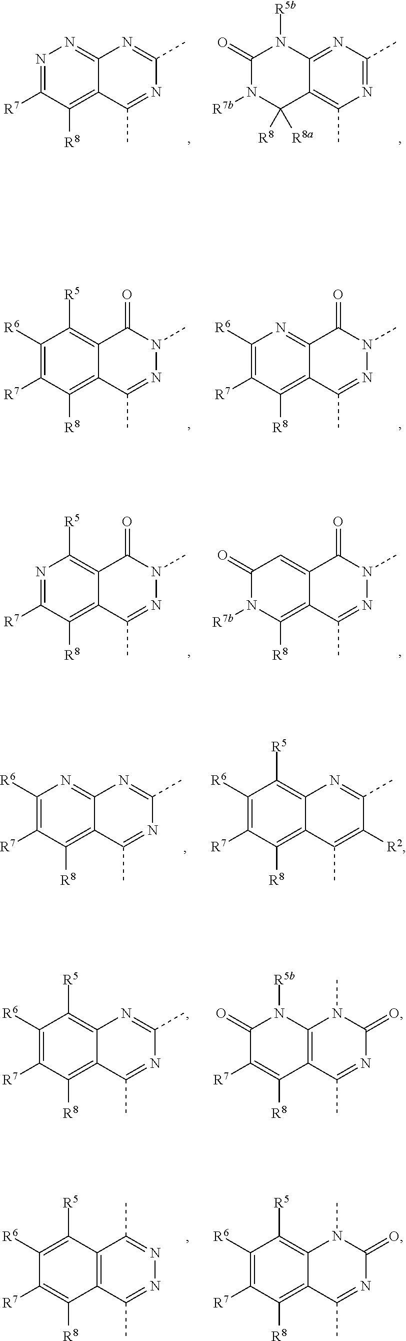

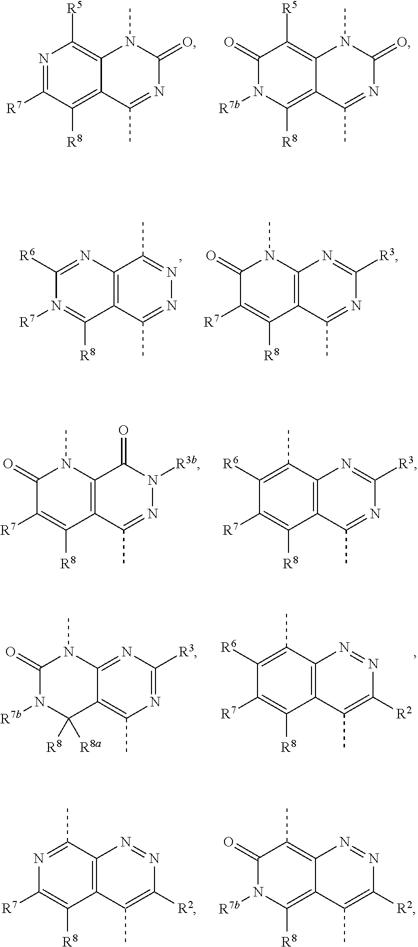

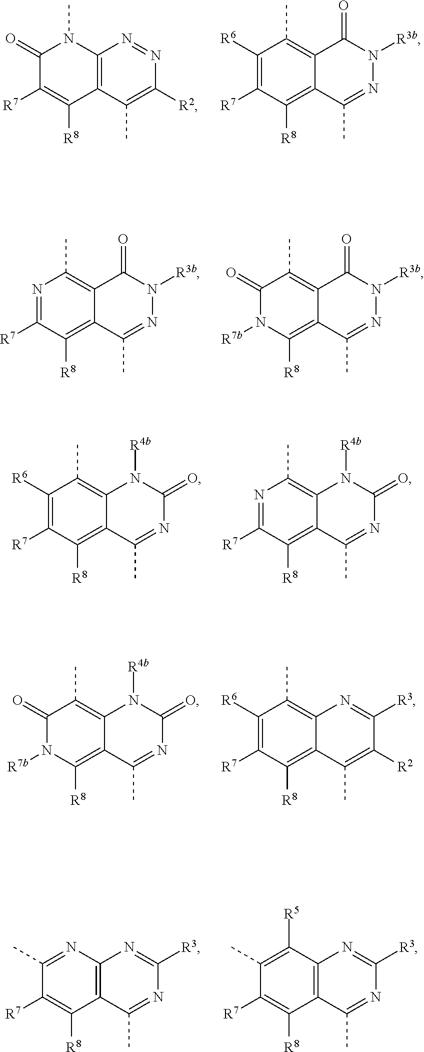

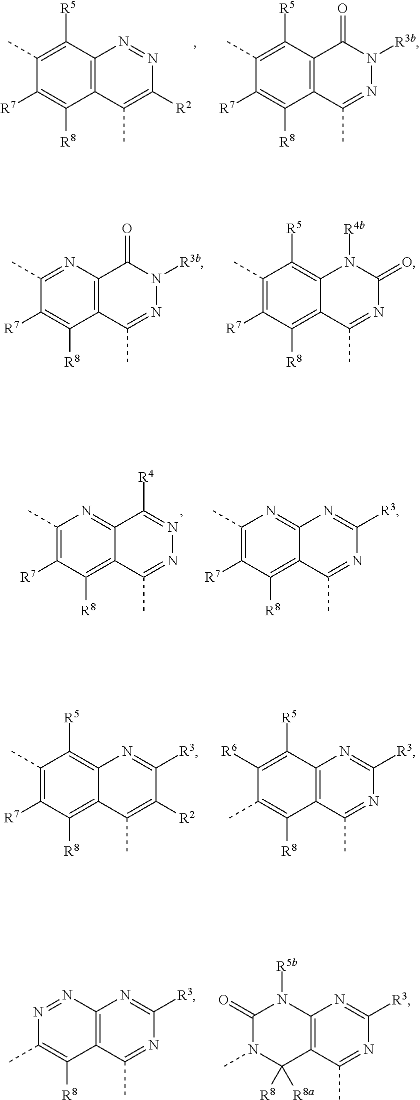

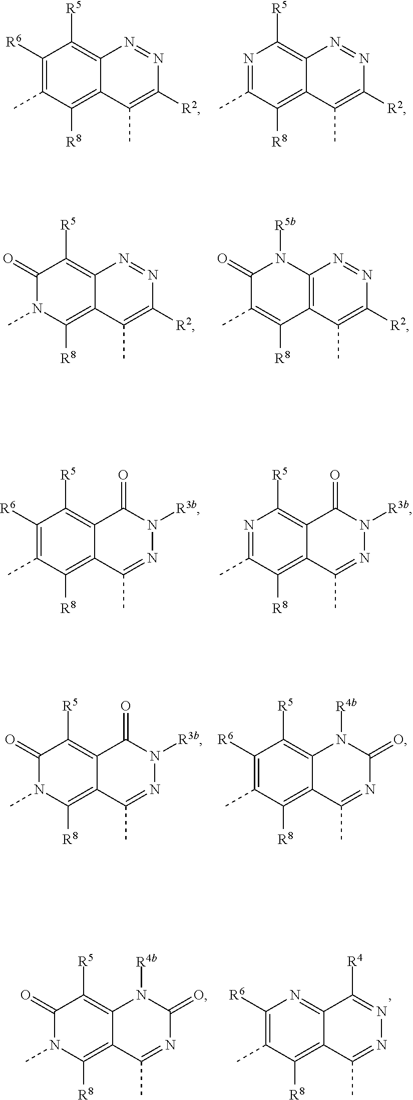

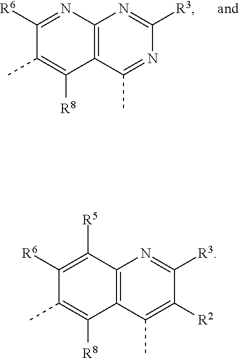

In some embodiments

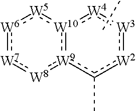

is selected from

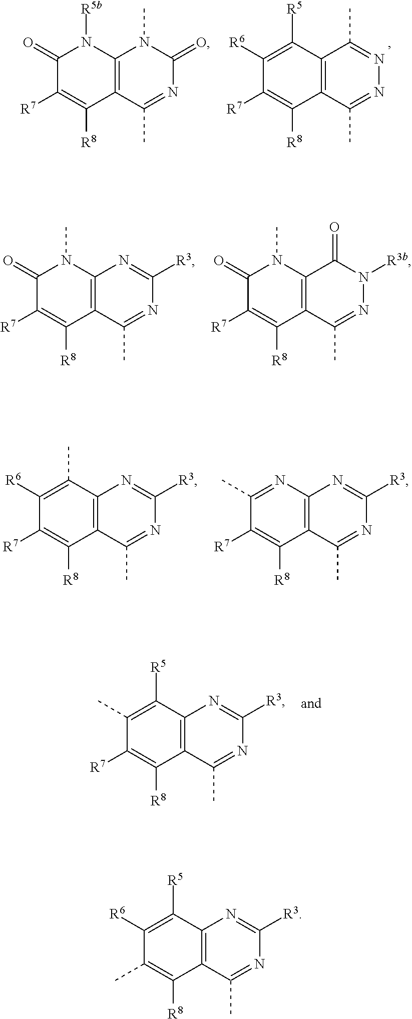

In some embodiments,

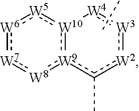

is selected from

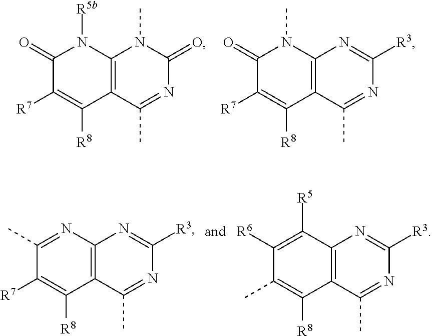

In some embodiments,

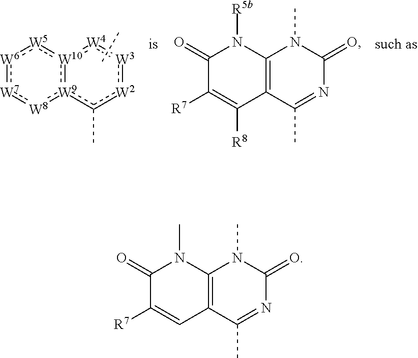

In some embodiments,

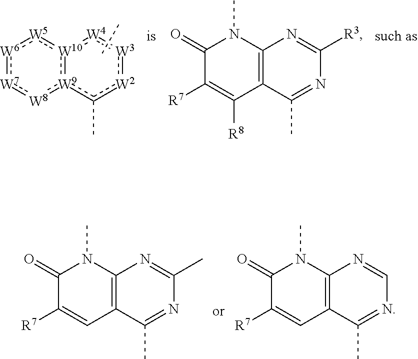

In some embodiments,

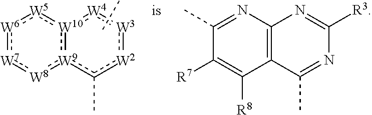

In some embodiments,

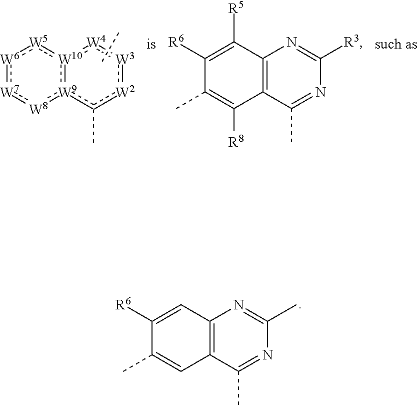

In some embodiments

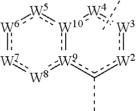

is selected from

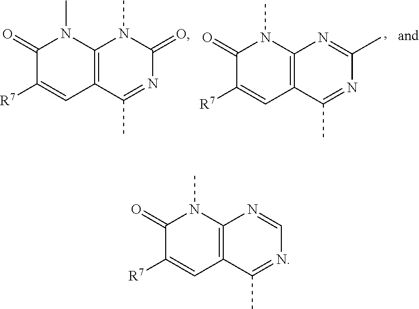

In some embodiments,

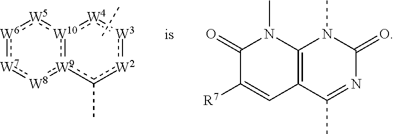

In some embodiments,

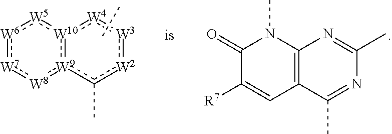

In some embodiments,

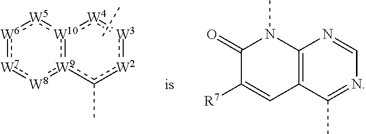

In some embodiments, for a compound of Formula (I), (I-A), (I-B), (I-C), (I-D), or (I-E), R², R^{2a}, R^{3a}, R^{4a}, R^{5a}, R⁶, R^{7a}, R⁸, and R^{8a} are each independently selected from hydrogen, halogen, —CN, C₁₋₃ alkyl, C₁₋₃ haloalkyl, —OH, —NH₂, —NHCH₃, and —N(CH₃)₂. In some embodiments, R², R^{2a}, R^{3a}, R^{4a}, R^{5a}, R⁶, R^{7a}, R⁸, and R^{8a} are each independently selected from hydrogen and —CH₃, such as hydrogen.

In some embodiments, for a compound of Formula (I), (I-A), (I-B), (I-C), (I-D), or (I-E), R³, R⁴, R⁵, and R⁶ are each independently selected from a bond to L², hydrogen, halogen, —CN, C₁₋₆ alkyl, C₃₋₆ carbocycle, 3- to 6-membered heterocycle, —OR¹², —SR¹², —N(R¹²)(R¹³), —C(O)OR¹², —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³), wherein each C₁₋₆ alkyl, C₃₋₆ carbocycle, and 3- to 6-membered heterocycle is independently optionally substituted with one, two, or three R²⁰; and R⁷ is selected from a bond to L², C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰. In some embodiments, R³, R⁴, R⁵, and R⁶ are each independently selected from a bond to L², hydrogen, halogen, —CN, C₁₋₃ alkyl, C₁₋₃ haloalkyl, —OH, —OCH₃, —NH₂, —NHCH₃, —N(CH₃)₂; and R⁷ is selected from a bond to L², C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰. In some embodiments, R³, R⁴, R⁵, and R⁶ are each independently selected from a bond to L², hydrogen, —CH₃, and —OCH₃; and R⁷ is selected from a bond to L², C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰.

In some embodiments, for a compound of Formula (I), (I-A), (I-B), (I-C), (I-D), or (I-E), R^{2b} and R^{8b} are each independently selected from hydrogen and C₁₋₃ alkyl, such as hydrogen and —CH₃. In some embodiments, R^{3b}, R^{4b}, R^{5b}, R^{6b}, and R^{7b} are each independently selected from a bond to L², hydrogen, and C₁₋₃ alkyl, such as a bond to L², hydrogen, and —CH₃. In some embodiments, R^{3b}, R^{4b}, R^{5b}, R⁶¹, and RV are each independently selected from a bond to L² and —CH₃. In some embodiments, R^{3b}, R^{4b}, R^{5b}, R^{6b}, and R^{7b} are each independently selected from a bond to L². In some embodiments, R⁹ and R¹⁰ are each hydrogen.

For a compound of Formula (I) wherein L² is -L³-D-L⁴-, it is understood that the selection of “a bond to L²” for one of R³, R⁴, R⁵, R⁶, R⁷, R^{3b}, R^{4b}, R^{5b}, R^{6b}, or R^{7b} inherently includes a bond to -L³-D-L⁴-, specifically to L⁴. For the avoidance of doubt, any recitation of R³, R⁴, R⁵, R⁶, R⁷, R^{3b}, R^{4b}, R^{5b}, R^{6b}, and/or R^{7b} that includes “a bond to L²” also may be considered to include “a bond to L⁴”.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), R¹ is selected from C₁₋₃ alkyl and C₁₋₃ haloalkyl, such as —CH₃, —CH₂CH₃, —CH(CH₃)₂, —CH₂F, —CHF₂, —CF₃, CH₂CH₂F, —CH₂CHF₂, and —CH₂CF₃. In some embodiments, R¹ is selected from C₁₋₃ alkyl, such as —CH₃ and —CH₂CH₃. In some embodiments, R¹ is —CH₃. In some embodiments, R¹ is (R)—CH₃. In some embodiments, R¹ is (S)—CH₃.

In some embodiments, for a compound of Formula (I), (I-B), (I-C), (I-C1), (I-C3), (I-D), (I-D1), (I-D2), (I-E), or (I-E1), R³ is selected from hydrogen, halogen, —CN, C₁₋₆ alkyl, C₃₋₆ carbocycle, 3- to 6-membered heterocycle, —OR¹², —SR¹², —N(R¹²)(R¹³), —C(O)OR¹², —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³), wherein each C₁₋₆ alkyl, C₃₋₆ carbocycle, and 3- to 6-membered heterocycle is independently optionally substituted with one, two, or three R²⁰. In some embodiments, R³ is selected from hydrogen, halogen, —CN, C₁₋₃ alkyl, C₁₋₃ haloalkyl, —OH, —OCH₃, —NH₂, —NHCH₃, —N(CH₃)₂. In some embodiments, R³ is selected from hydrogen, halogen, —CN, —OR¹², and C₁₋₆ alkyl optionally substituted with one, two, or three R²⁰. In some embodiments, R³ is C₁₋₆ alkyl optionally substituted with one, two, or three R²⁰. In some embodiments, R³ is hydrogen or —CH₃. In some embodiments, R³ is hydrogen. In some embodiments, R³ is —CH₃.

In some embodiments, for a compound of Formula (I), (I-B), (I-C), (I-C2), (I-D), or (I-E), R^{3b} is selected from hydrogen and C₁₋₃ alkyl, such as hydrogen and —CH₃. In some embodiments, R^{3b} is —CH₃. In some embodiments, R^{3b} is hydrogen.

In some embodiments, for a compound of Formula (I), (I-A), (I-B), (I-B2), (I-D), (I-D2), (I-E), or (I-E1), R⁵ is selected from hydrogen, halogen, —CN, C₁₋₆ alkyl, C₃₋₆ carbocycle, 3- to 6-membered heterocycle, —OR¹², —SR¹², —N(R¹²)(R¹³), —C(O)OR¹², —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³), wherein each C₁₋₆ alkyl, C₃₋₆ carbocycle, and 3- to 6-membered heterocycle is independently optionally substituted with one, two, or three R²⁰. In some embodiments, R⁵ is selected from hydrogen, halogen, —CN, C₁₋₃ alkyl, C₁₋₃ haloalkyl, —OH, —OCH₃, —NH₂, —NHCH₃, —N(CH₃)₂. In some embodiments, R⁵ is selected from hydrogen, —OR¹², and C₁₋₆ alkyl optionally substituted with one, two, or three R²⁰. In some embodiments, R⁵ is hydrogen or —CH₃. In some embodiments, R⁵ is hydrogen. In some embodiments, R⁵ is —CH₃.

In some embodiments, for a compound of Formula (I), (I-A), (I-B), (I-B1), (I-D), or (I-E), R^{5b} is selected from hydrogen and C₁₋₃ alkyl, such as hydrogen and —CH₃. In some embodiments, R^{5b} is selected from hydrogen and C₁₋₆ alkyl optionally substituted with one, two, or three R²⁰. In some embodiments, R^{5b} is —CH₃. In some embodiments, R^{5b} is hydrogen. In some embodiments, R^{5b} is a bond to L².

In some embodiments, for a compound of Formula (I), (I-A), (I-B), (I-B2), (I-C), (I-C3), (I-E), or (I-E1), R⁶ is selected from hydrogen, halogen, —CN, C₁₋₆ alkyl, C₃₋₆ carbocycle, 3- to 6-membered heterocycle, —OR¹², —SR¹², —N(R¹²)(R¹³), —C(O)OR¹², —C(O)R¹⁵, —S(O)R¹⁵, —OC(O)R¹⁵, —C(O)N(R¹²)(R¹³), —N(R¹⁴)C(O)R¹⁵, —S(O)₂R¹⁵, and —S(O)₂N(R¹²)(R¹³), wherein each C₁₋₆ alkyl, C₃₋₆ carbocycle, and 3- to 6-membered heterocycle is independently optionally substituted with one, two, or three R²⁰. In some embodiments, R⁶ is selected from hydrogen, halogen, —CN, C₁₋₃ alkyl, C₁₋₃ haloalkyl, —OH, —OCH₃, —NH₂, —NHCH₃, and —N(CH₃)₂. In some embodiments, R⁶ is selected from hydrogen, —OR¹², and C₁₋₆ alkyl optionally substituted with one, two, or three R²⁰, and wherein R¹² is selected from C₁₋₆ alkyl. In some embodiments, R⁶ is selected from hydrogen and —OCH₃. In some embodiments, R⁶ is hydrogen. In some embodiments, R⁶ is —OCH₃.

In some embodiments, for a compound of Formula (I), (I-A), (I-B), (I-B1), (I-B2), (I-C), (I-C1), (I-C2), (I-C3), (I-D), (I-D1), or (I-D2), R⁷ is selected from C₁₋₆ alkyl, C₃₋₁₀ carbocycle, 3- to 10-membered heterocycle, —OR¹², —SR¹², —N(R¹²)(R¹³), —C(O)R¹⁵, —C(O)N(R¹²)(R¹³), —N(R¹⁴)C(O)R¹⁵, —S(O)₂R¹⁵, —S(O)₂N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₃₋₁₀ carbocycle, and 3- to 10-membered heterocycle is independently optionally substituted with one, two, or three R²⁰. In some embodiments, R⁷ is selected from C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰. In some embodiments, R⁷ is selected from C₁₋₆ alkyl, C₃₋₁₀ cycloalkyl, 3- to 10-membered heterocycloalkyl, and —N(R¹²)(R¹³), wherein C₁₋₆ alkyl, C₃₋₁₀ cycloalkyl, and 3- to 10-membered heterocycloalkyl are optionally substituted with one, two, or three R²⁰. In some embodiments, R⁷ is 3- to 10-membered heterocycloalkyl optionally substituted with one, two, or three R²⁰, such as R⁷ is 4- to 6-membered heterocycloalkyl optionally substituted with one, two, or three R²⁰. In some embodiments, R⁷ is 3- to 10-membered heterocycloalkyl optionally substituted with one, two, or three R²⁰, wherein the heterocycloalkyl comprises at least one O, N, or S, such as one O atom, one or two N atoms, or one S atom. In some embodiments, R⁷ is 3- to 6-membered heterocycloalkyl optionally substituted with one, two, or three R²⁰, wherein the heterocycloalkyl comprises S(O)₂. In some embodiments, R⁷ is C₃₋₁₀ cycloalkyl optionally substituted with one, two, or three R²⁰, such as R⁷ is C₃₋₆ cycloalkyl optionally substituted with one, two, or three R²⁰. In some embodiments, R⁷ is C₃₋₄ cycloalkyl optionally substituted with one R²⁰, optionally wherein R²⁰ is —CN. In some embodiments, R⁷ is C₁₋₆ alkyl optionally substituted with one, two, or three R²⁰, such as R⁷ is C₁₋₆ alkyl substituted with one or two R²⁰. In some embodiments, R⁷ is —N(R¹²)(R¹³). In some embodiments, R⁷ is —OR¹², such as —O(3- to 6-membered heterocycloalkyl). In some embodiments, R⁷ is substituted with at least one —CN. In some embodiments, R⁷ is unsubstituted. In some embodiments, R⁷ is selected from C₃₋₁₀ carbocycle and 3- to 10-membered heterocycle, each of which is optionally substituted with one, two, or three R²⁰. In some embodiments, R⁷ is selected from C₃₋₁₀ carbocycle and 3- to 10-membered heterocycle, each of which is optionally substituted with one, two, or three substituents selected from oxo, —CN, and C₁₋₆ alkyl. In some embodiments, R⁷ is 4- to 6-membered heterocycloalkyl substituted with one, two, or three substituents selected from oxo, —CN, and C₁₋₃ alkyl. In some embodiments, R⁷ is C₃₋₄ cycloalkyl substituted with one, two, or three substituents selected from oxo, —CN, and C₁₋₃ alkyl.

In some embodiments, for a compound of Formula (I), (I-A), (I-B), (I-B1), (I-B2), (I-C), (I-C1), (I-C2), (I-C3), (I-D), (I-D1), or (I-D2), R⁷ is selected from C₁₋₆ alkyl, C₃₋₁₀ carbocycle, 3- to 10-membered heterocycle, —OR¹², —SR¹², —N(R¹²)(R¹³), —C(O)R¹⁵, —C(O)N(R¹²)(R¹³), —N(R¹⁴)C(O)R¹⁵, —S(O)₂R¹⁵, —S(O)(NR¹²)R¹⁵, —S(O)₂N(R¹²)(R¹³), —CH₂C(O)N(R¹²)(R¹³), —CH₂N(R¹⁴)C(O)R¹⁵, —CH₂S(O)₂R¹⁵, —CH₂S(O)(NR¹²)R¹⁵, and —CH₂S(O)₂N(R¹²)(R¹³), wherein each C₁₋₆ alkyl, C₃₋₁₀ carbocycle, and 3- to 10-membered heterocycle is independently optionally substituted with one, two, or three R²⁰. In some embodiments, R⁷ is selected from C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, —S(O)(NR¹²)R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰. In some embodiments, R⁷ is 3- to 6-membered heterocycloalkyl optionally substituted with one, two, or three R²⁰, wherein the heterocycloalkyl comprises S(O)(NR¹²).

In some embodiments, for a compound of Formula (I), (I-A), (I-B), (I-B1), (I-B2), (I-C), (I-C1), (I-C2), (I-C3), (I-D), (I-D1), or (I-D2), R⁷ is

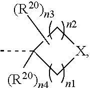

wherein:
n1 is an integer from 1 to 3;
n2 is an integer from 0 to 2;
n3 is an integer from 0 to 2;
n4 is 0 or 1; and
X is selected from —O—, —S(O₂)—, —P(O)—, —CH₂—, —CH(OH)—, —CH(OR¹²)—, —CH(R²⁰)—, —C(R²⁰)₂—, —NR¹²—, —CH(N(R¹²)(R¹³))—, —CH(C(O)N(R¹²)(R¹³))—, and —CH(S(O)₂N(R¹²)(R¹³))—,
wherein R¹², R¹³, and R²⁰ are as defined elsewhere herein, and
optionally wherein two R²⁰ groups, or R²⁰ and R¹², join together with the atom(s) to which they are attached to form a ring. For example, RT may be selected from

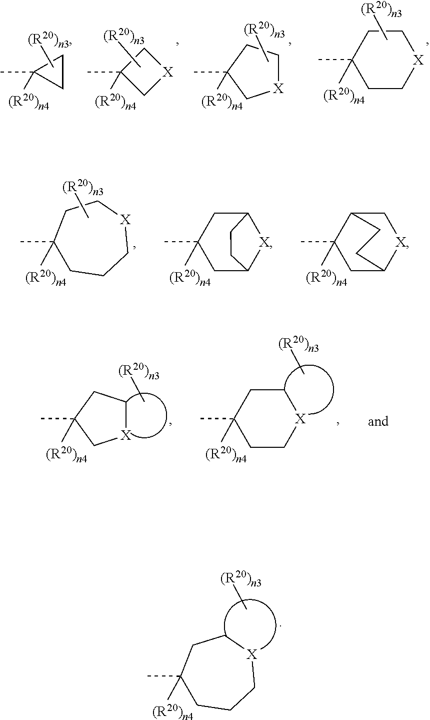

In some embodiments, X is selected from —O—, —S(O)₂—, —S(O)(NR¹²)—, —P(O)—, —CH₂—, —CH(OH)—, —CH(OR¹²)—, —CH(R²⁰)—, —C(R²⁰)₂—, —NR¹²—, —CH(N(R¹²)(R¹³))—, —CH(C(O)N(R¹²)(R¹³))—, and —CH(S(O)₂N(R¹²)(R¹³))—.

In some embodiments, for a compound of Formula (I), (I-A), (I-B), (I-B1), (I-B2), (I-C), (I-C1), (I-C2), (I-C3), (I-D), (I-D1),

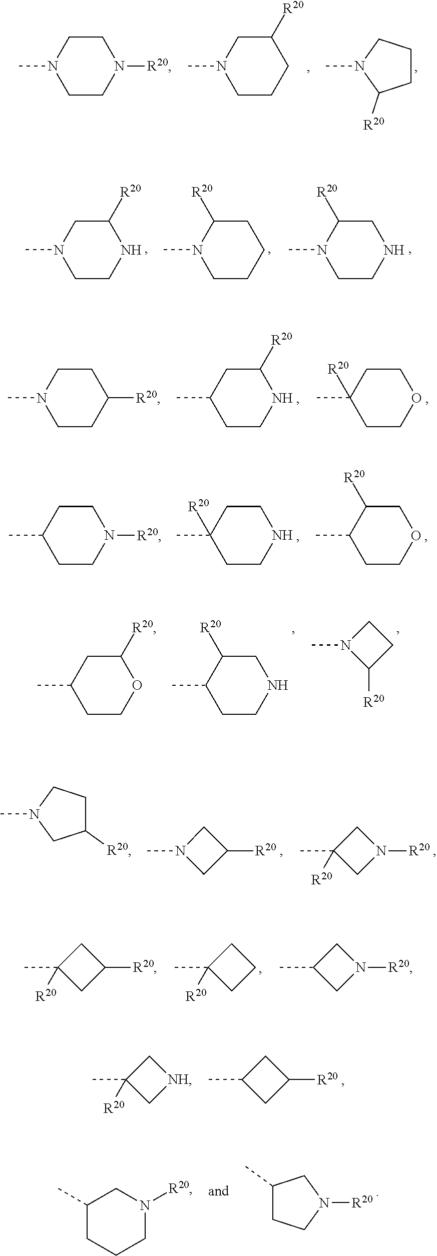

In some embodiments, R⁷ is selected from

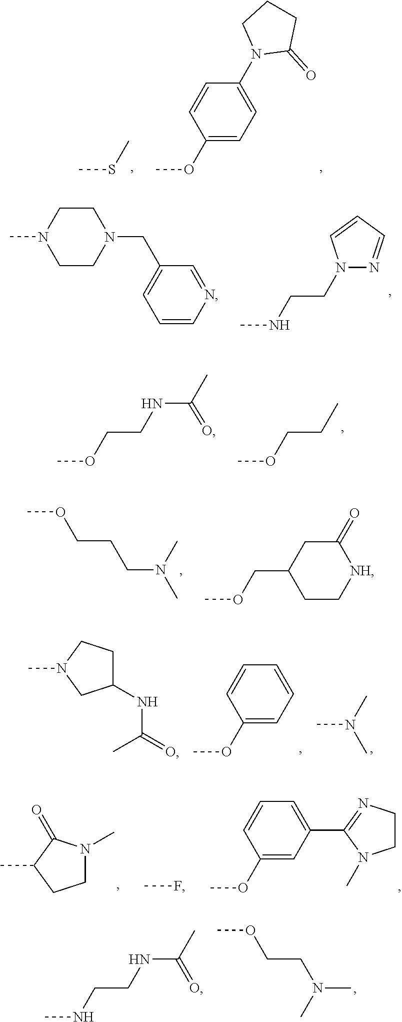

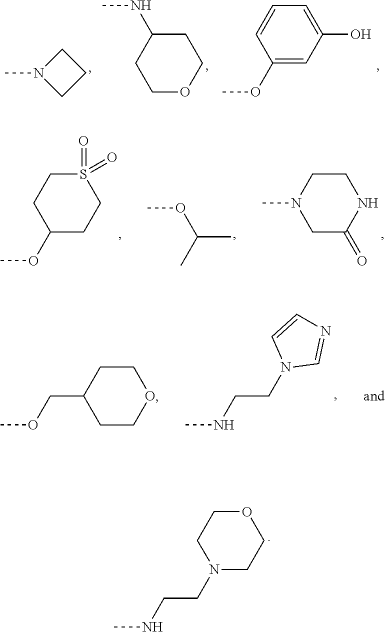

In some embodiments, R⁷ is selected from

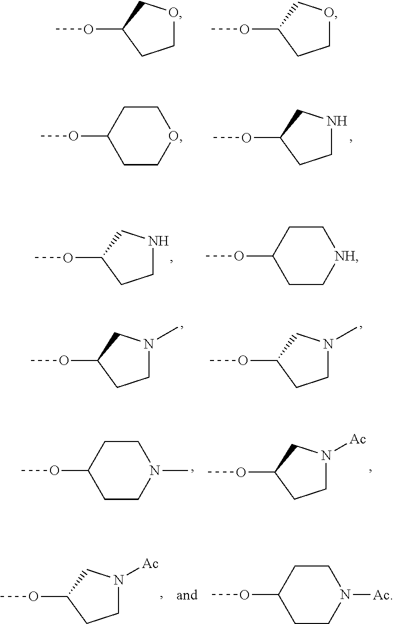

In some embodiments, R⁷ is selected from

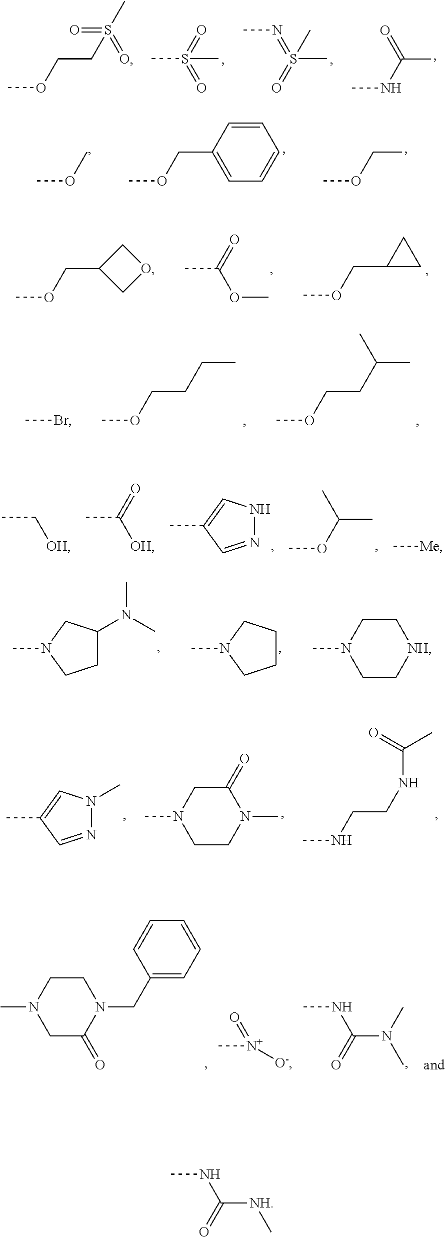

In some embodiments, R⁷ is selected from

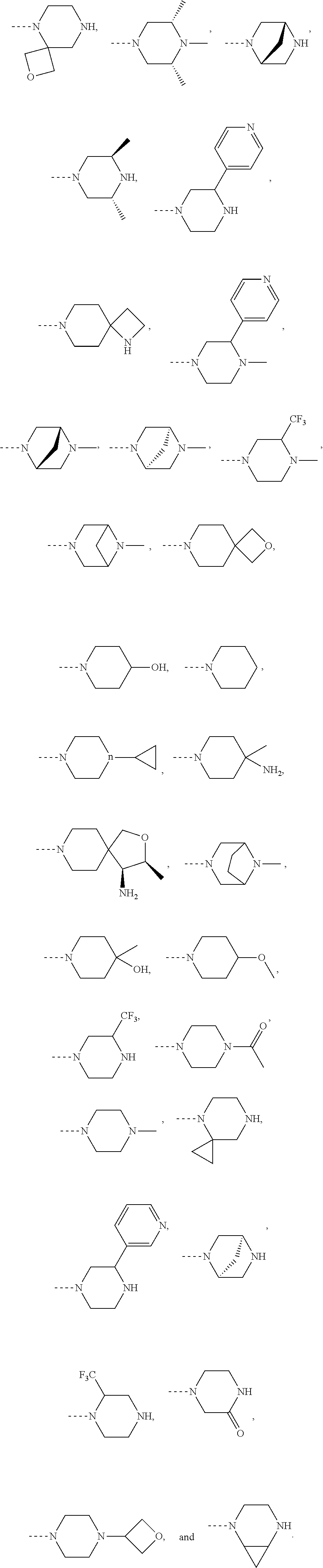

In some embodiments, R⁷ is selected from

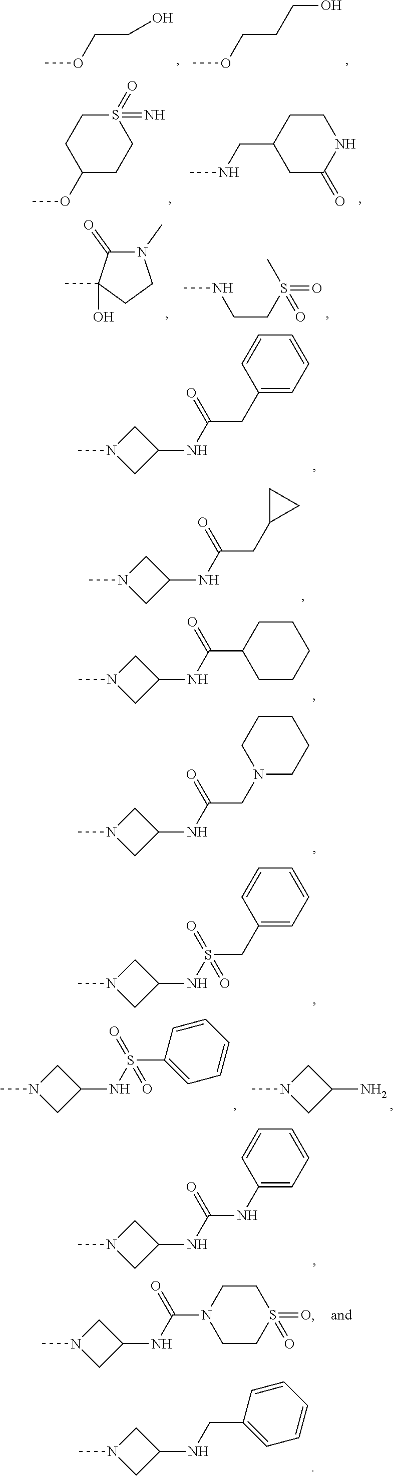

In some embodiments, R⁷ is selected from

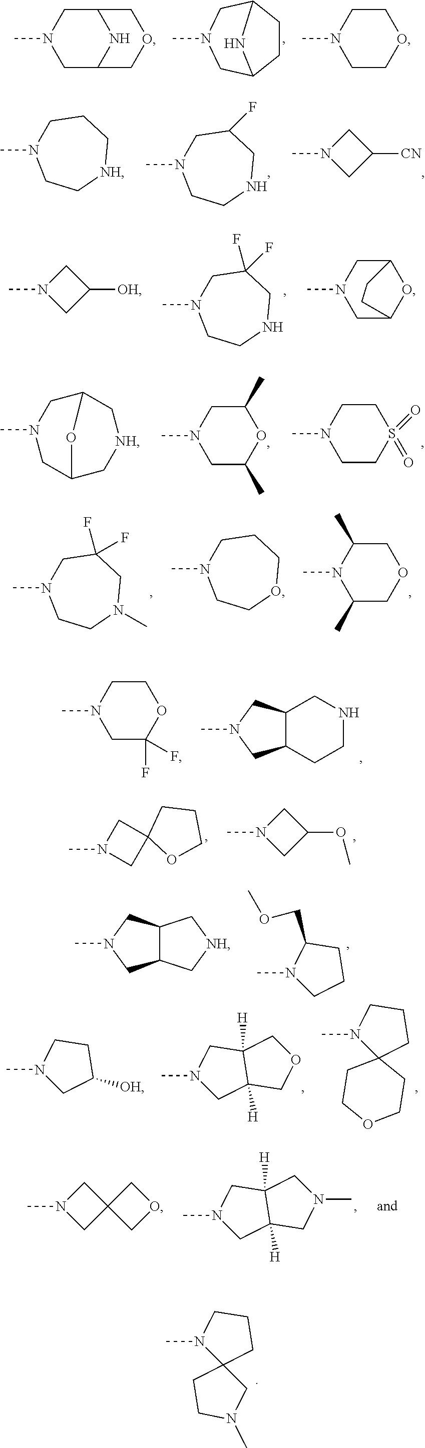

In some embodiments R⁷ is selected from

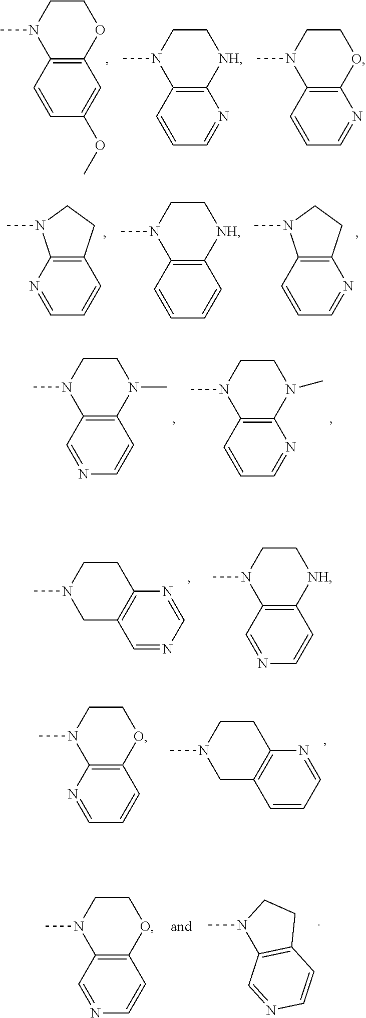

In some embodiments, R⁷ is selected from

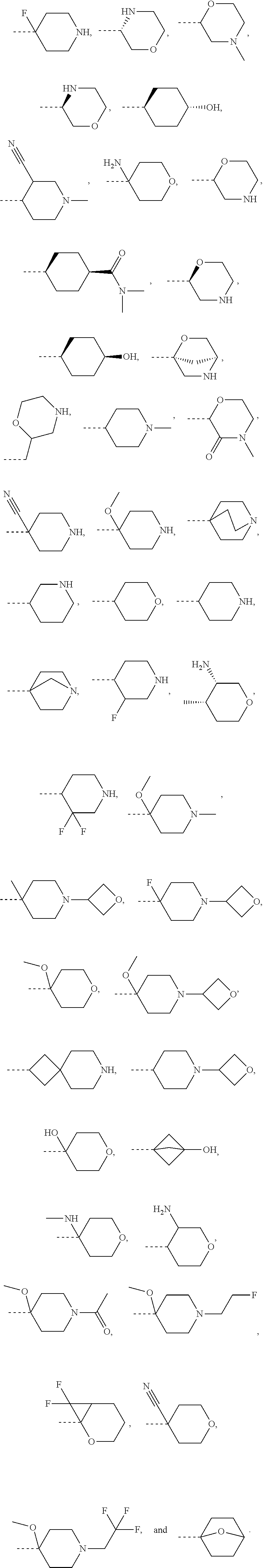

In some embodiments, R⁷ is selected from

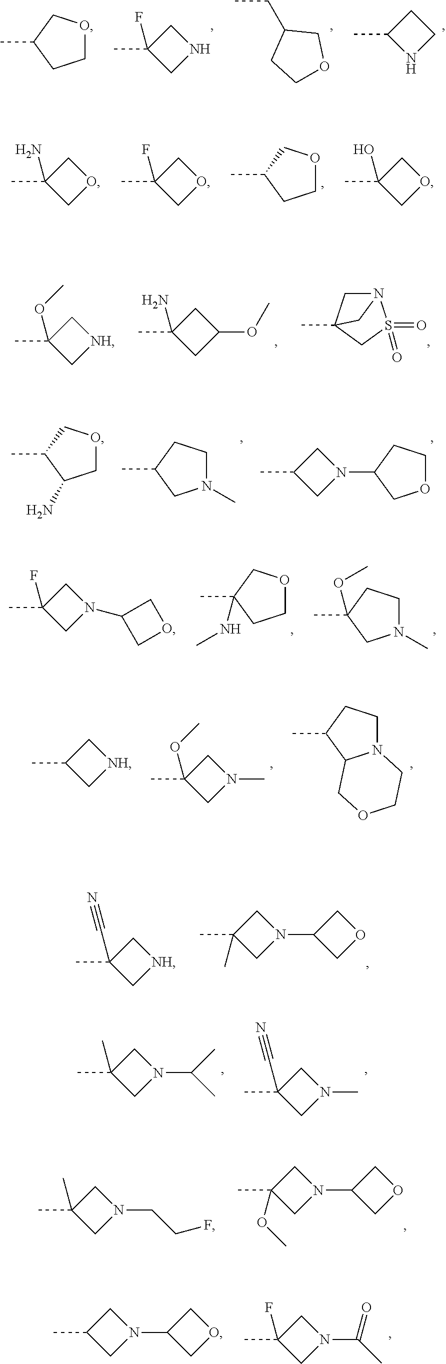

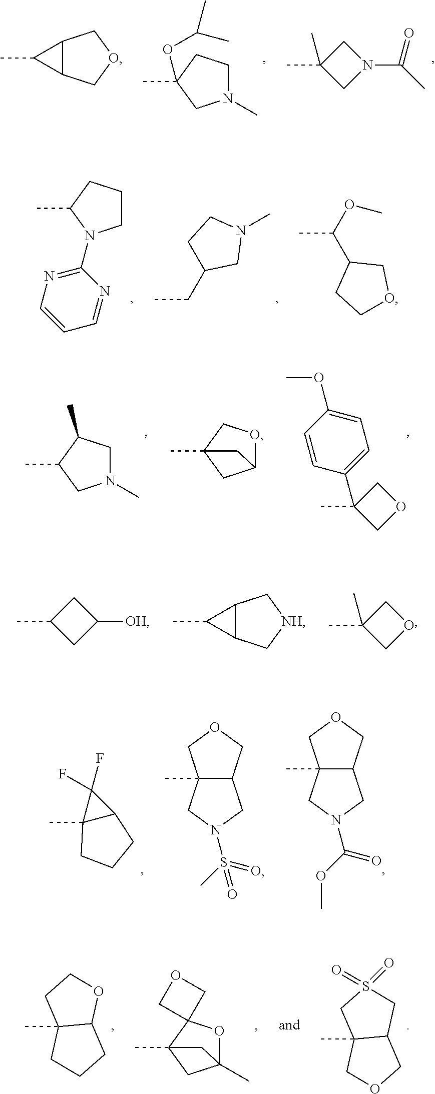

In some embodiments, R⁷ is selected from

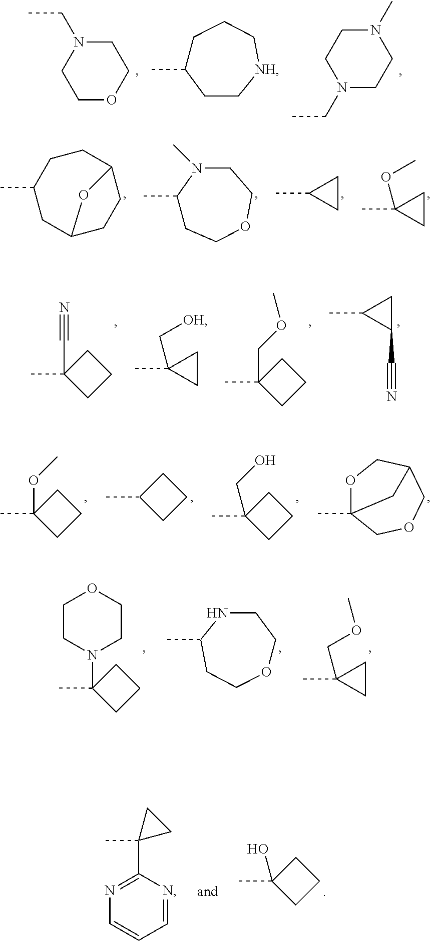

In some embodiments, R⁷ is selected from

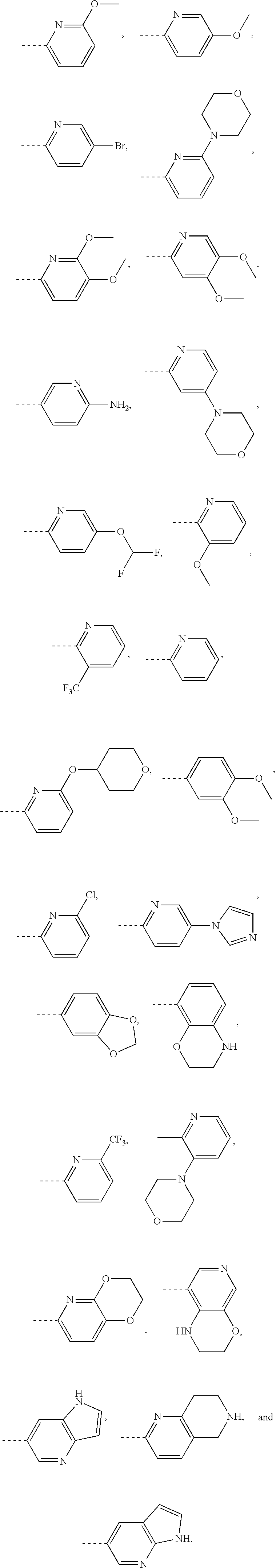

In some embodiments, R⁷ is selected from

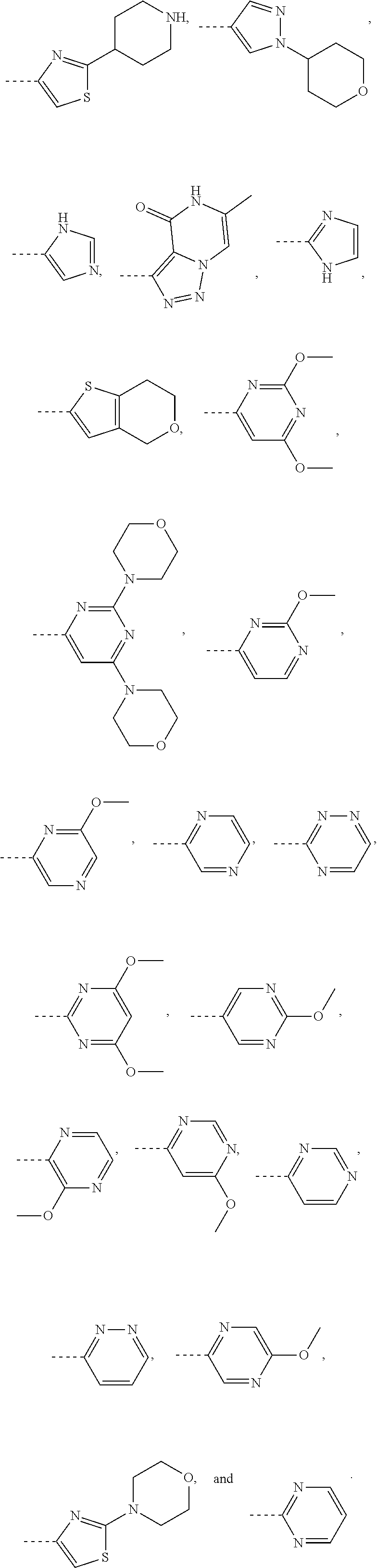

In some embodiments, R⁷ is selected from

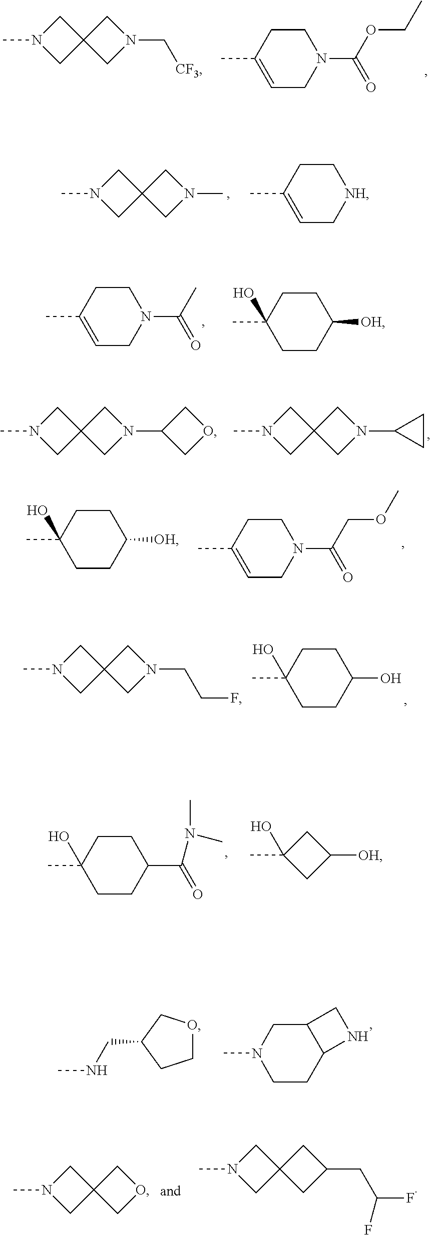

In some embodiments, R⁷ is selected from

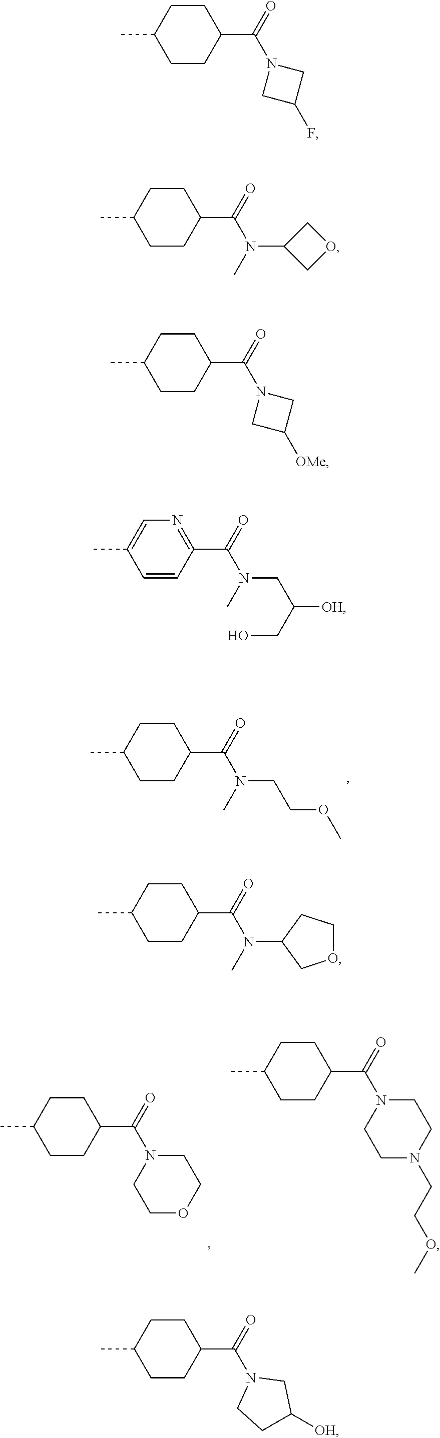

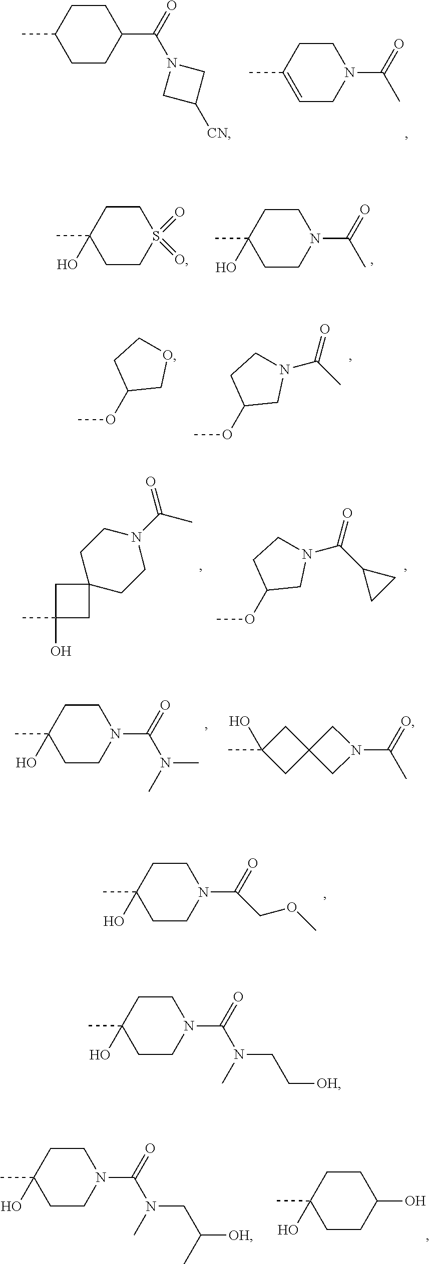

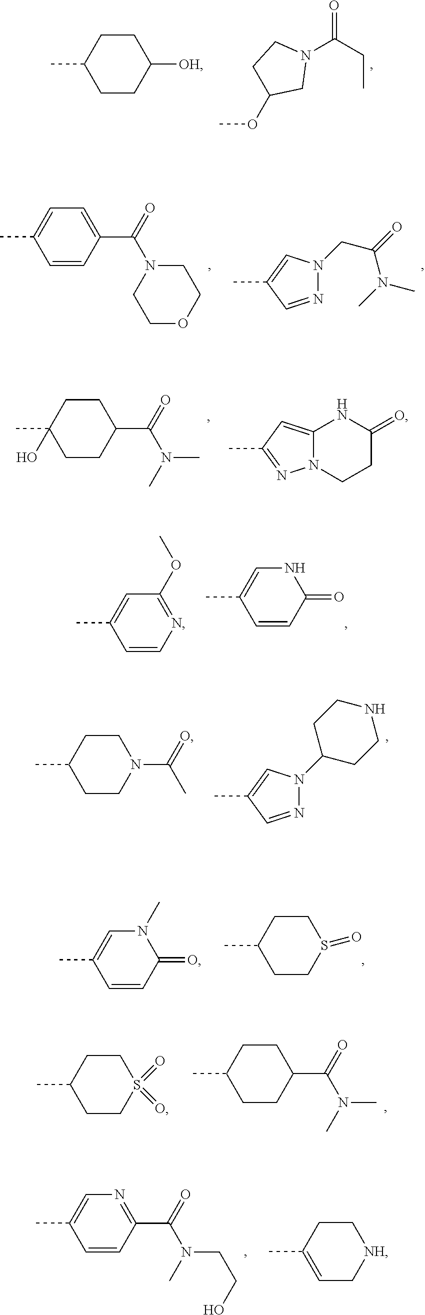

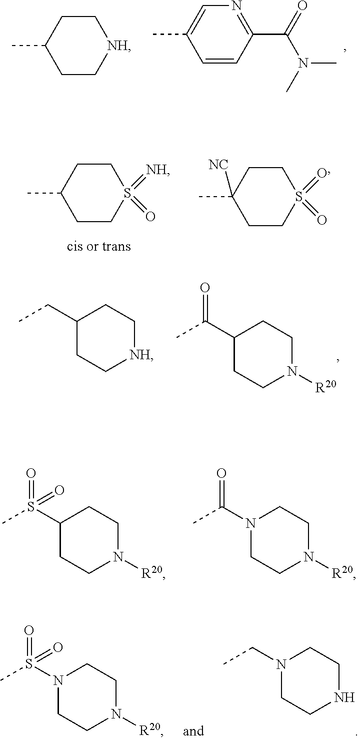

In some embodiments, R⁷ is

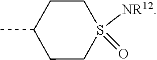

In some embodiments, R⁷ is selected from

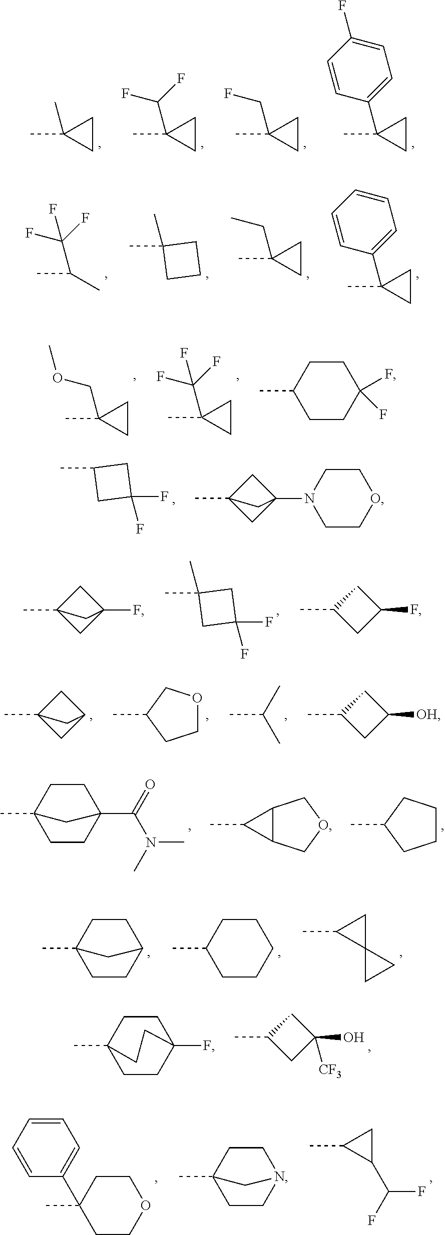

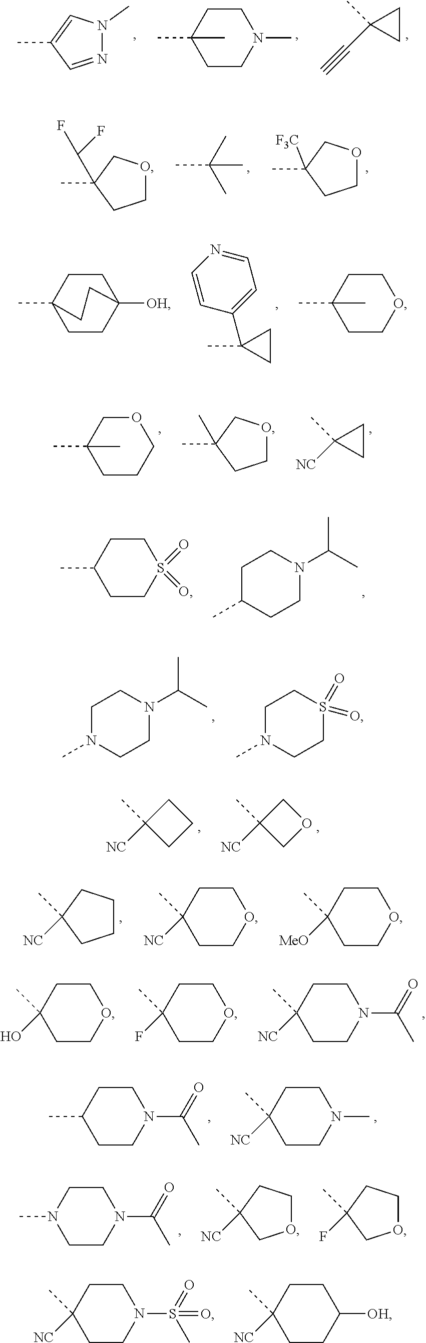

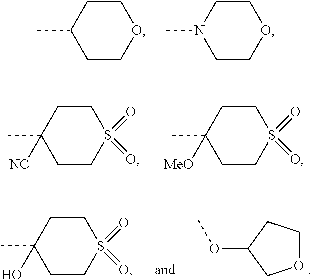

In some embodiments, R⁷ is selected from

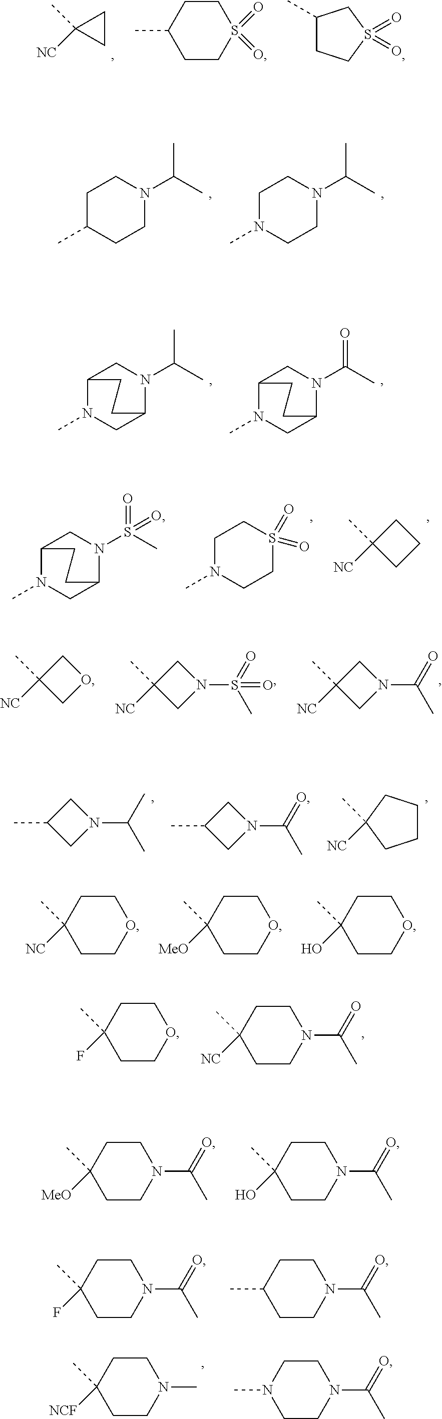

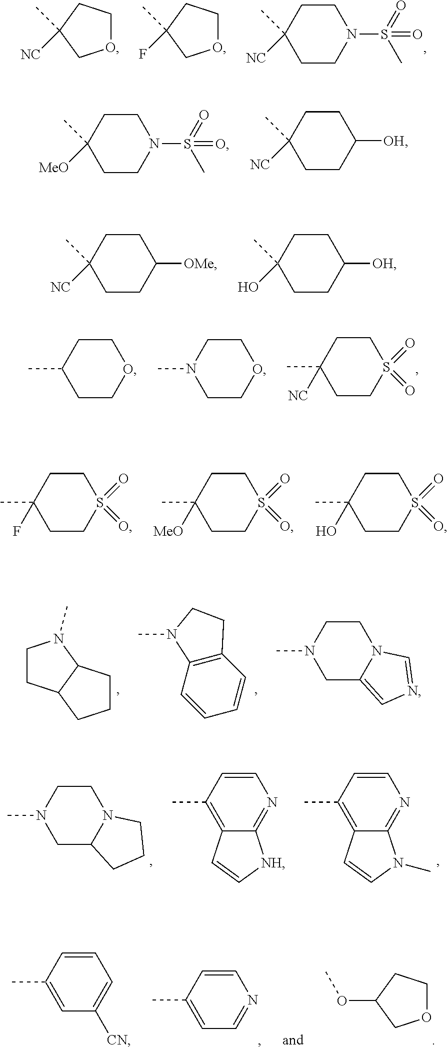

In some embodiments R⁷ is selected from

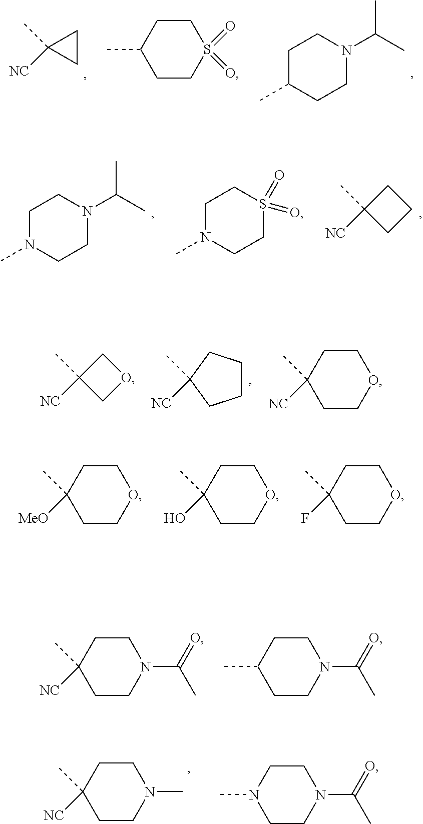

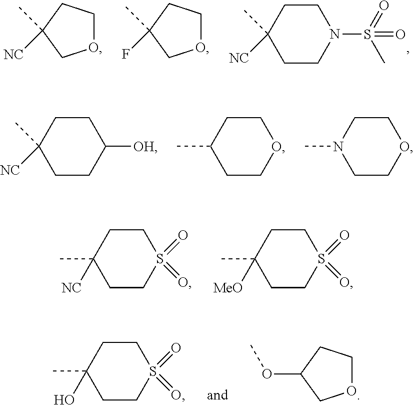

In some embodiments, R⁷ is selected from

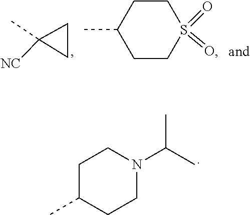

In some embodiments R⁷ is

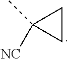

In some embodiments, R⁷ is

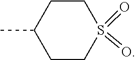

In some embodiments, R⁷ is

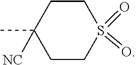

In some embodiments, R⁷ is

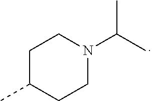

In some embodiments, R⁷ is

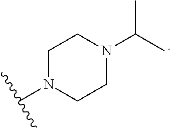 [SMILES: *N1CCN(C(C)C)CC1]

In some embodiments, R⁷ is

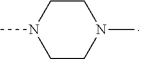

In some embodiments, R⁷ is

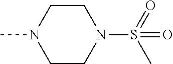

In some embodiments, R⁷ is

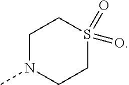

In some embodiments, R⁷ is

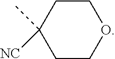

In some embodiments, R⁷ is

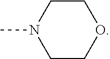

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), R⁸ is selected from hydrogen, halogen, and C₁₋₆ alkyl optionally substituted with one, two, or three R²⁰. In some embodiments, R⁸ is hydrogen.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1),

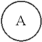

is selected from C₃₋₁₀ carbocycle and 3- to 10-membered heterocycle, such as C₅₋₇ carbocycle and 5- to 7-membered heterocycle, each of which is optionally substituted with one or more R¹¹. In some embodiments,

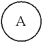

is selected from C₅₋₇ cycloalkyl, 5- to 7-membered heterocycloalkyl, 5- to 7-membered heteroaryl, and phenyl, each of which is optionally substituted with one or more R¹¹. In some embodiments,

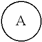

is selected from phenyl, pyridyl, and thiophenyl, each of which is optionally substituted with one or more R¹¹. In some embodiments,

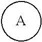

is selected from

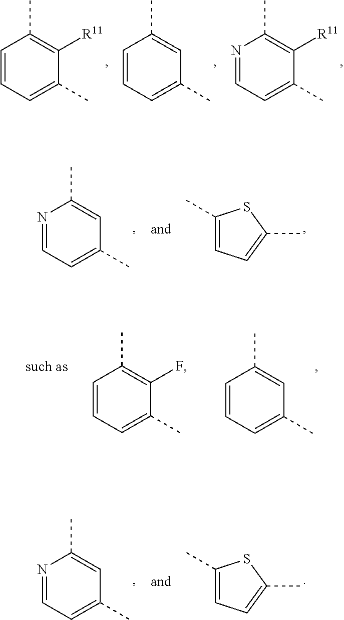

In some embodiments, R¹¹, when present, is independently selected at each occurrence from fluorine and —CH₃. In some embodiments,

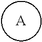

is selected from,

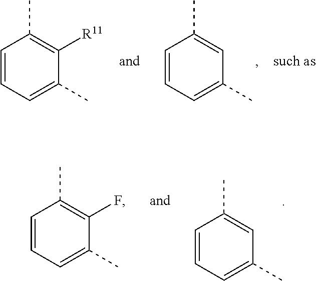

In some embodiments,

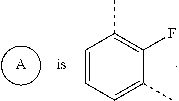

In some embodiments is

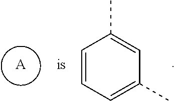

In some embodiments,

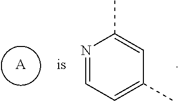

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), L¹ is selected from C₁₋₆ alkylene and C₁₋₆ haloalkylene, such as C₁₋₃ alkylene and C₁₋₃ haloalkylene. In some embodiments, L¹ is selected from a bond and C₁₋₃ haloalkylene. In some embodiments, L¹ is C₁₋₃ haloalkylene, such as —CF₂—, —CF₂CH₂—, or —CF₂CH₂CH₂—. In some embodiments, L¹ is C₁₋₂ haloalkylene, such as —CF₂— or —CF₂CH₂—. In some embodiments, L¹ is —CF₂—. In some embodiments, L¹ is —CF₂CH₂—. In some embodiments, L¹ is —CF₂CH₂CH₂—. In some embodiments, L¹ is a bond. In some embodiments, L¹ is selected from a bond, —O—, —NR¹²—, —S—, C₁₋₆ alkylene, C₁₋₆ haloalkylene, and 2- to 6-membered heteroalkylene, wherein C₁₋₆ alkylene, C₁₋₆ haloalkylene, and 2- to 6-membered heteroalkylene are optionally substituted with one or more R^{11b}. In some embodiments, L¹ is selected from —O—, —NR¹²—, —S—, and 2- to 6-membered heteroalkylene, wherein 2- to 6-membered heteroalkylene is optionally substituted with one or more R^{11b}. In some embodiments, L¹ is C₁₋₆ alkylene optionally substituted with one or more R^{11b}, such as one, two, or three R^{11b}. In some embodiments, L¹ is selected from a bond, —O—, —NR¹²—, —S—, C₁₋₃ alkylene, C₁₋₃ haloalkylene, and 2- to 3-membered heteroalkylene, wherein C₁₋₃ alkylene, C₁₋₃ haloalkylene, and 2- to 3-membered heteroalkylene are optionally substituted with one or more R^{11b}. In some embodiments, L¹ is selected from —O—, —NR¹²—, —S—, and 2- to 3-membered heteroalkylene, wherein 2- to 3-membered heteroalkylene is optionally substituted with one or more R^{11b}. In some embodiments, L¹ is C₁₋₃ alkylene substituted with one or more R^{11b}, such as one, two, or three R^{11b}.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1),

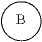

is absent or selected from C₄₋₈ carbocycle and 4- to 8-membered heterocycle, each of which is optionally substituted with one or more R^{11a}. In some embodiments,

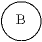

is absent or selected from phenyl and 4- to 8-membered heterocycle, each of which is optionally substituted with one or more R^{11a}. In some embodiments,

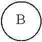

is selected from phenyl and 4- to 8-membered heterocycle, each of which is optionally substituted with one or more R^{11a}. In some embodiments,

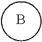

is absent or selected from phenyl, azetidine, pyrrolidine, and piperidine, each of which is optionally substituted with one or more R^{11a}. In some embodiments,

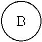

is selected from phenyl, azetidine, pyrrolidine, and piperidine, each of which is optionally substituted with one or more R^{11a}. In some embodiments,

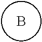

is selected from azetidine, pyrrolidine, and piperidine, each of which is optionally substituted with one or more —CH₃. In some embodiments,

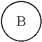

is phenyl, optionally substituted with one or more R^{11a}. In some embodiments,

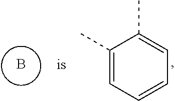

optionally substituted with one or more R^{11a}. In some embodiments is

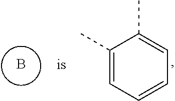

In some embodiments,

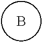

is azetidine, optionally substituted with one or more R^{11a}. In some embodiments,

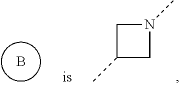

optionally substituted with one or more R^{11a}. In some embodiments,

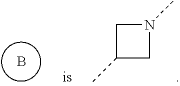

In some embodiments,

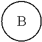

is pyrrolidine, optionally substituted with one or more R^{11a}. In some embodiments,

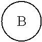

is piperidine, optionally substituted with one or more R^{11a}. In some embodiments, is

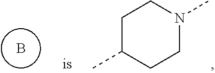

optionally substituted with one or more R^{11a}. In some embodiments,

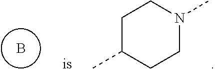

In some embodiments,

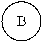

is unsubstituted. In some embodiments,

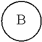

is substituted with one or more R^{11a}, such as one, two or three R^{11a}. In some embodiments, R^{11a} is independently selected at each occurrence from halogen, —CN, C₁₋₆ alkyl, C₃₋₆ carbocycle, 3- to 6-membered heterocycle, —OR¹², —N(R¹²)(R¹³), —N(R¹⁴)S(O)₂R¹⁵, —C(O)N(R¹²)(R¹³), —N(R¹⁴)C(O)R¹⁵, —S(O)₂R¹⁵, and —S(O)₂N(R¹²)(R¹³), wherein C₁₋₆ alkyl, C₃₋₆ carbocycle, and 3- to 6-membered heterocycle are optionally substituted with one, two, or three R²⁰. In some embodiments, R^{11a} is independently selected at each occurrence from halogen, C₁₋₆ alkyl, and C₁₋₆ haloalkyl. In some embodiments, R^{11a} is independently selected at each occurrence from C₁₋₆ alkyl. In some embodiments, R^{11a} is —CH₃.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}. In some embodiments, L² is selected from C₅₋₂₅ alkylene, C₅₋₂₅ alkenylene, 5- to 25-membered heteroalkylene, and 5- to 25-membered heteroalkenylene, each of which is optionally substituted with one or more R^{11b}. In some embodiments, L², together with the atoms to which it is attached, forms a 16- to 36-membered macrocyclic ring, such as a 16- to 24-membered macrocyclic ring. In some embodiments, L² is selected from C₆₋₅ alkylene, C₆₋₁₅ alkenylene, C₆₋₁₅ alkynylene, 6- to 15-membered heteroalkylene, and 6- to 15-membered heteroalkenylene, each of which is optionally substituted with one or more R^{11b}. In some embodiments, L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}. In some embodiments, an alkenylene or heteroalkenylene of L² comprises one carbon-carbon double bond. In some embodiments, a heteroalkylene or heteroalkenylene of L² comprises at least one oxygen or nitrogen atom. In some embodiments, a heteroalkylene or heteroalkenylene of L² comprises at least one basic nitrogen. In some embodiments, L² is selected from C₅₋₉ alkylene, C₅₋₉ alkenylene, and 5- to 9-membered heteroalkylene, each of which is optionally substituted with one or more R^{11b}. In some embodiments, L² is selected from C₆₋₉ alkylene, C₆₋₉ alkenylene, and 6- to 9-membered heteroalkylene, each of which is optionally substituted with one or more R^{11b}. In some embodiments, L² is selected from C₅₋₈ alkylene, C₅₋₈ alkenylene, and 5- to 8-membered heteroalkylene, each of which is optionally substituted with one or more R^{11b}. In some embodiments, L² is selected from C₆₋₈ alkylene, C₆₋₈ alkenylene, and 6- to 8-membered heteroalkylene, each of which is optionally substituted with one or more R^{11b}. In some embodiments, L² is selected from C₆₋₈ alkylene and C₆₋₈ alkenylene, each of which is optionally substituted with one or more R^{11b}. In some embodiments, L² is selected from C₆ alkylene and C₆ alkenylene, each of which is optionally substituted with one or more R^{11b}. In some embodiments, L² is selected from C₇ alkylene and C₇ alkenylene, each of which is optionally substituted with one or more R^{11b}. In some embodiments, L² is selected from C₈ alkylene and C₈ alkenylene, each of which is optionally substituted with one or more R^{11b}. In some embodiments, L² is —CH₂CHCH(CH₂)₄—. In some embodiments, L² is 6- to 8-membered heteroalkylene, optionally substituted with one or more R^{11b}. In some embodiments, L² is 6-membered heteroalkylene, optionally substituted with one or more R^{11b}. In some embodiments, L² is 7-membered heteroalkylene, optionally substituted with one or more R^{11b} In some embodiments, L² is 8-membered heteroalkylene, optionally substituted with one or more R^{11b}. In some embodiments, L² is 8-membered heteroalkylene, wherein the heteroalkylene comprises one oxygen atom. In some embodiments, L² is —(CH₂)₂₋₅O(CH₂)₀₋₅—, such as L² is —(CH₂)₂₋₅O(CH₂)₂₋₅—. In some embodiments, L² is —(CH₂)₄O(CH₂)₃—. In some embodiments, R^{11b} is independently selected at each occurrence from halogen, oxo, C₁₋₆ alkyl, C₁₋₆ haloalkyl, (C₁₋₆ alkyl)-OH, and —OH. In some embodiments, R^{11b} is independently selected at each occurrence from —F, ═O, —CH₃, —CH₂F, —CHF₂, —CF₃, —CH₂CH₂F, —CH₂CHF₂, —CH₂OH, and —OH. In some embodiments, R^{11b} is independently selected at each occurrence from —CH₃, —CH₂OH, —CH_{2F}, —CHF₂, and —CF₃, or two R^{11b} join to form ═O or C₃₋₆ cycloalkyl, such as cyclopropyl. In some embodiments, R^{11b} is independently selected at each occurrence from —CH₃, —CH₂OH, —CH₂F, —CHF₂, and —CF₃, or two R^{11b} join to form C₃₋₆ cycloalkyl, such as cyclopropyl. In some embodiments, R^{11b} is independently selected at each occurrence from —CH₃, —F, —CN, and —OH. In some embodiments, L² comprises —C(O)N(R¹⁴)— or —N(R¹⁴)C(O)—. In some embodiments, L² comprises —O—. In some embodiments, L² is substituted with at least one —CH₃, —CH₂OH, —CH₂F, —CHF₂, or —CF₃, or two substituents join to form cyclopropyl. In some embodiments, L² is substituted with at least one —CH₃, —F, —CN, or —OH. In some embodiments, L² is unsubstituted.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), L² is —(C₁₋₅ alkylene)-C(O)N(R¹⁴)—(C₁₋₅ alkylene)-, such as —(C₁₋₅ alkylene)-C(O)N(CH₃)—(C₁₋₅ alkylene)- or —(C₁₋₅ alkylene)-C(O)NH—(C₁₋₅ alkylene)-, wherein C₁₋₅ alkylene is optionally substituted with one or more R^{11b}. In some embodiments, L² is —(C₁₋₂ alkylene)-C(O)N(R¹⁴)—(C₃₋₄ alkylene)-, such as —(C₁₋₂ alkylene)-C(O)N(CH₃)—(C₃₋₄ alkylene)- or —(C₁₋₂ alkylene)-C(O)NH—(C₃₋₄ alkylene)-, wherein C₁₋₂ alkylene and C₃₋₄ alkylene are each independently optionally substituted with one or more R^{11b}. In some embodiments, R^{11b} is independently selected at each occurrence from halogen, C₁₋₆ alkyl, C₁₋₆ haloalkyl, (C₁₋₆ alkyl)-OH, and —OH. In some embodiments, R^{11b} is independently selected at each occurrence from —F, —CH₃, —CH₂F, —CHF₂, —CF₃, —CH₂CH₂F, —CH₂CHF₂, —CH₂OH, and —OH. In some embodiments, R^{11b} is independently selected at each occurrence from —CH₃, —CH₂OH, —CH₂F, —CHF₂, and —CF₃, or two R^{11b} join to form C₃₋₆ cycloalkyl, such as cyclopropyl. In some embodiments, R^{11b} is independently selected at each occurrence from —CH₃, —F, —CN, and —OH. In some embodiments, L² is substituted with at least one —CH₃, —F, —CN, or —OH.

In some embodiments, L² is selected from —C(R^{11b})(R^{11b})—(C₃₋₁₀ alkylene)-C(R^{11b})(R^{11b})—, —CH(R^{11b})—(C₃₋₁₀ alkylene)-C(R^{11b})(R^{11b})—, —CH₂—(C₃₋₁₀ alkylene)-C(R^{11b})(R^{11b})—, —CH(R^{11b})—(C₃₋₁₀ alkylene)-CH(R^{11b})—, —CH₂—(C₃₋₁₀ alkylene)-CH(R^{11b})—, —C(R^{11b})(R^{11b})—(C₃₋₁₀ alkenylene)-C(R^{11b})(R^{11b})—, —CH(R^{11b})—(C₃₋₁₀ alkenylene)-C(R^{11b})(R^{11b})—, —CH₂—(C₃₋₁₀ alkenylene)-C(R^{11b})(R^{11b})—, —CH(R^{11b})—(C₃₋₁₀ alkenylene)-CH(R^{11b})—, —CH₂—(C₃₋₁₀ alkenylene)-CH(R^{11b})—, —C(R^{11b})(R^{11b})-(3- to 10-membered heteroalkylene)-C(R^{11b})(R^{11b})—, —CH(R^{11b})-(3- to 10-membered heteroalkylene)-C(R^{11b})(R^{11b})—, —CH₂-(3- to 10-membered heteroalkylene)-C(R^{11b})(R^{11b})—, —CH(R^{11b})-(3- to 10-membered heteroalkylene)-CH(R^{11b})—, —CH₂-(3- to 10-membered heteroalkylene)-CH(R^{11b})—, —C(R^{11b})(R^{11b})-(3- to 10-membered heteroalkenylene)-C(R^{11b})(R^{11b})—, —CH(R^{11b})-(3- to 10-membered heteroalkenylene)-C(R^{11b})(R^{11b})—, —CH₂-(3- to 10-membered heteroalkenylene)-C(R^{11b})(R^{11b})—, —CH(R^{11b})-(3- to 10-membered heteroalkenylene)-CH(R^{11b})—, and —CH₂-(3- to 10-membered heteroalkenylene)-CH(R^{11b})—. In some embodiments, L² is selected from —C(R^{11b})(R^{11b})—(C₃₋₁₀ alkylene)-C(R^{11b})(R^{11b})—, —CH(R^{11b})—(C₃₋₁₀ alkylene)-C(R^{11b})(R^{11b})—, —CH₂—(C₃₋₁₀ alkylene)-C(R^{11b})(R^{11b})—, —CH(R^{11b})—(C₃₋₁₀ alkylene)-CH(R^{11b})—, and —CH₂—(C₃₋₁₀ alkylene)-CH(R^{11b})—. In some embodiments, L² is selected from —C(R^{11b})(R^{11b})—(C₃₋₁₀ alkenylene)-C(R^{11b})(R^{11b})—, —CH(R^{11b})—(C₃₋₁₀ alkenylene)-C(R^{11b})(R^{11b})—, —CH₂—(C₃₋₁₀ alkenylene)-C(R^{11b})(R^{11b})—, —CH(R^{11b})—(C₃₋₁₀ alkenylene)-CH(R^{11b})—, and —CH₂—(C₃₋₁₀ alkenylene)-CH(R^{11b})—. In some embodiments, L² is selected from —C(R^{11b})(R^{11b})-(3- to 10-membered heteroalkylene)-C(R^{11b})(R^{11b})—, —CH(R^{11b})-(3- to 10-membered heteroalkylene)-C(R^{11b})(R^{11b})—, —CH₂-(3- to 10-membered heteroalkylene)-C(R^{1b})(R^{11b})—, —CH(R^{11b})-(3- to 10-membered heteroalkylene)-CH(R^{11b})—, and —CH₂-(3- to 10-membered heteroalkylene)-CH(R^{11b})—. In some embodiments, L² is selected from —C(R^{11b})(R^{11b})-(3- to 10-membered heteroalkenylene)-C(R^{11b})(R^{11b})—, —CH(R^{11b})-(3- to 10-membered heteroalkenylene)-C(R^{11b})(R^{11b})—, —CH₂-(3- to 10-membered heteroalkenylene)-C(R^{11b})(R^{11b})—, —CH(R^{11b})-(3- to 10-membered heteroalkenylene)-CH(R^{11b})—, and —CH₂-(3- to 10-membered heteroalkenylene)-CH(R^{11b})—. Any C₃₋₁₀ alkylene, C₃₋₁₀ alkenylene, 3- to 10-membered heteroalkylene, or 3- to 10-membered heteroalkenylene in this paragraph may optionally be substituted with one or more R^{11b}. In some embodiments, R^{11b} is independently selected at each occurrence from halogen, oxo, C₁₋₆ alkyl, C₁₋₆ haloalkyl, (C₁₋₆ alkyl)-OH, and —OH. In some embodiments, R^{11b} is independently selected at each occurrence from —F, —CH₃, —CH₂F, —CHF₂, —CF₃, —CH₂CH₂F, —CH₂CHF₂, —CH₂OH, and —OH. In some embodiments, R^{11b} is independently selected at each occurrence from —CH₃, —CH₂OH, —CH₂F, —CHF₂, and —CF₃, or two R^{11b} join to form ═O or C₃₋₆ cycloalkyl, such as cyclopropyl.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1), L² is -L³-D-L⁴-, wherein L³ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b}; D is absent or selected from C₃₋₁₂ carbocycle and 3- to 12-membered heterocycle, each of which is optionally substituted with one or more R^{11d}; and L⁴ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b}. As noted above, alkenylene and alkynylene groups comprise one or more carbon-carbon double or triple bonds, respectively (i.e., comprising two or more carbon atoms, for example as in C₂₋₁₀ alkenylene, C₂₋₁₀ alkynylene, C₂₋₈ alkenylene, C₂₋₈ alkynylene, C₅₋₂₅ alkenylene, C₅₋₂₅ alkynylene, C₆₋₁₅ alkenylene, C₆₋₁₅ alkynylene, C₅₋₁₀ alkenylene, and C₅₋₁₀ alkynylene). Similarly, a heteroalkenylene group comprises one or more carbon-carbon double bond (i.e., comprising two or more carbon atoms and one or more heteroatom, for example as in 3- to 10-membered heteroalkenylene, 3- to 8-membered heteroalkenylene, 5- to 25-membered heteroalkenylene, 6- to 15-membered heteroalkenylene, and 5- to 10-membered heteroalkenylene. In some embodiments, L² is -L³-D-L⁴-, wherein L³ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b}; D is selected from C₃₋₁₂ carbocycle and 3- to 12-membered heterocycle, each of which is optionally substituted with one or more R^{11d}; and L⁴ is absent. In some embodiments, L² is -L³-D-L⁴-, wherein L³ is selected from C₁₋₈ alkylene, C₂₋₈ alkenylene, 2- to 8-membered heteroalkylene, and 3- to 8-membered heteroalkenylene, each of which is optionally substituted with one or more R^{11b}; D is absent or selected from C₃₋₁₀ carbocycle and 3- to 10-membered heterocycle, each of which is optionally substituted with one or more R^{11d}; and L⁴ is selected from C₁₋₈ alkylene, C₂₋₈ alkenylene, 2- to 8-membered heteroalkylene, and 3- to 8-membered heteroalkenylene, each of which is optionally substituted with one or more R¹¹. In some embodiments, L² is -L³-D-L⁴-, wherein L³ is selected from C₁₋₈ alkylene, C₂₋₈ alkenylene, 2- to 8-membered heteroalkylene, and 3- to 8-membered heteroalkenylene, each of which is optionally substituted with one or more R^{11b}; D is selected from C₃₋₁₀ carbocycle and 3- to 10-membered heterocycle, each of which is optionally substituted with one or more R^{11d}; and L⁴ is selected from C₁₋₈ alkylene, C₂₋₈ alkenylene, 2- to 8-membered heteroalkylene, and 3- to 8-membered heteroalkenylene, each of which is optionally substituted with one or more R^{11b}. In some embodiments, R^{11b} is independently selected at each occurrence from halogen, oxo, C₁₋₆ alkyl, C₁₋₆ haloalkyl, (C₁₋₆ alkyl)-OH, and —OH. In some embodiments, R^{11b} is independently selected at each occurrence from —F, —CH₃, —CH₂F, —CHF₂, —CF₃, —CH₂CH₂F, —CH₂CHF₂, —CH₂OH, and —OH. In some embodiments, R^{11b} is independently selected at each occurrence from —CH₃, —CH₂OH, —CH₂F, —CHF₂, and —CF₃, or two R^{11b} join to form ═O or C₃₋₆ cycloalkyl, such as cyclopropyl. In some embodiments, D is selected from phenyl and 5- to 8-membered heteroaryl, such as triazole and imidazole. In some embodiments, D is unsubstituted. In some embodiments, D is substituted with one or more R^{11d}, such as one, two, or three R^{11d}.

In some embodiments, for a compound of Formula (I), L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷. For example, L² may be covalently bound to W³—wherein R^{3b} or R³ is a bond to L²—as depicted in Formula (I-A). In some embodiments, L² is covalently bound to W⁴—wherein R^{4b} or R⁴ is a bond to L²—as depicted in Formula (I-B). In some embodiments, L² is covalently bound to W⁵—wherein R^{5b} or R⁵ is a bond to L²—as depicted in Formula (I-C). In some embodiments, L² is covalently bound to W⁶—wherein R^{6b} or R⁶ is a bond to L²—as depicted in Formula (I-D). In some embodiments, L² is covalently bound to W⁷—wherein R^{7b} or R⁷ is a bond to L²—as depicted in Formula (I-E).

In some embodiments, for a compound of Formula (I), (I-A), (I-B), (I-1), (I-B1), (I-B2), (I-C), (I-C1), (I-C2), (I-C3), (I-D), (I-D1), (I-D2), (I-E), or (I-E1):
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, and —N(R²²)(R²³);
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, for a compound of Formula (I), (I-A), (I-B), (I-1), (I-B1), (I-B2), (I-C), (I-C1), (I-C2), (I-C3), (I-D), (I-D1), (I-D2), (I-E), or (I-E1), R²⁰ is independently selected at each occurrence from halogen, oxo, ═NR²², —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)(NR²²)R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NR²², —CN, C₁₋₆ alkyl, C₁₋₆ haloalkyl, C₁₋₆ alkoxy, C₁₋₆ haloalkoxy, —OR²¹, —SR²¹, and —N(R²²)(R²³).

In some embodiments, for a compound of Formula (I), (I-A), (I-B), (I-1), (I-B1), (I-B2), (I-C), (I-C1), (I-C2), (I-C3), (I-D), (I-D1), (I-D2), (I-E), or (I-E1), R²⁰ is independently selected at each occurrence from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

In some embodiments, for a compound of Formula (I-B1), R^{5b} is —CH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen. In some embodiments, for a compound of Formula (I-B2), R⁵ is hydrogen; R⁶ is selected from hydrogen and —OCH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen.

In some embodiments, for a compound of Formula (I-C1), R³ is hydrogen or —CH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen. In some embodiments, for a compound of Formula (I-C2), R^{3b} is —CH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen. In some embodiments, for a compound of Formula (I-C3), R³ is hydrogen or —CH₃; R⁶ is selected from hydrogen and —OCH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1): R¹ is —CH₃;

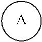

is selected from phenyl and 5- to 7-membered heteroaryl, each of which is optionally substituted with one or more R¹¹; L¹ is selected from a bond and C₁₋₃ haloalkylene;

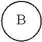

is selected from absent, phenyl, and 4- to 8-membered heterocycle, wherein the phenyl and 4- to 8-membered heterocycle are optionally substituted with one or more R^{11a}; and L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}. In some embodiments, R¹¹, when present, is fluorine; R^{11a}, when present, is —CH₃; and R^{11b}, when present, is selected from halogen, oxo, C₁₋₆ alkyl, C₁₋₆ haloalkyl, (C₁₋₆ alkyl)-OH, and —OH.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1): R¹ is —CH₃;

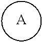

is selected from

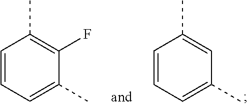

L¹ is C₁₋₂ haloalkylene;

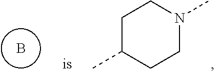

optionally substituted with one or more R^{11a}; and L² is selected from C₆₋₈ alkylene, C₆₋₈ alkenylene, and 6- to 8-membered heteroalkylene, each of which is optionally substituted with one or more R^{11b}. In some embodiments, R¹ is —CH₃;

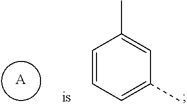

L¹ is C₁₋₂ haloalkylene;

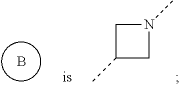

and L² is 6- to 8-membered heteroalkylene, optionally substituted with one or more R^{11b}. In some embodiments, R¹ is —CH₃;

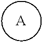

is selected from

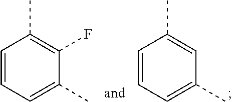

L¹ is C₁₋₂ haloalkylene;

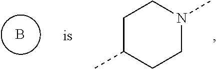

optionally substituted with one or more R^{11a}; and L² is selected from C₆₋₈alkylene and C₆₋₈ alkenylene, each of which is optionally substituted with one or more R^{11b}. In some embodiments, R¹ is —CH₃;

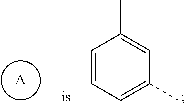

L¹ is C₁₋₂ haloalkylene;

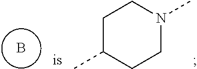

and L² is C₆₋₈ alkenylene, optionally substituted with one or more R^{11b}. In some embodiments, R¹ is —CH₃;

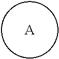

is selected from

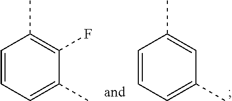

L¹ is C₁₋₂ haloalkylene;

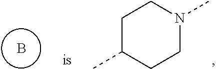

optionally substituted with one or more R^{11a}; and L² is —(C₁₋₂ alkylene)-C(O)N(CH₃)—(C₃₋₄ alkylene)- or —(C₁₋₂ alkylene)-C(O)NH—(C₃₋₄ alkylene)-, wherein C₁₋₂ alkylene and C₃₋₄ alkylene are each independently optionally substituted with one or more R^{11b}. In some embodiments, R^{11a}, when present, is —CH₃; and R^{11b}, when present, is selected from halogen, —CN, oxo, C₁₋₃ alkyl, C₁₋₃ haloalkyl, (C₁₋₃ alkyl)-OH, and —OH.

In some embodiments, for a compound of Formula (I), (I-A), (I-B), (I-B1), (I-B2), (I-C), (I-C1), (I-C2), (I-C3), (I-D), (I-D1), (I-D2), (I-E), or (I-E1): R¹ is —CH₃;

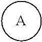

is selected from

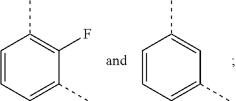

L¹ is C₁₋₂ haloalkylene;

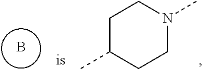

and L² is selected from C₆₋₈ alkylene, C₆₋₈ alkenylene, and 6- to 8-membered heteroalkylene, each of which is optionally substituted with one or more R^{11b}. In some embodiments, R¹ is —CH₃;

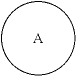

is selected from

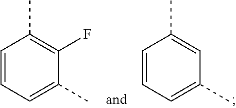

L¹ is C₁₋₂ haloalkylene;

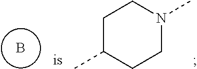

and L² is selected from C₆₋₈ alkylene and C₆₋₈ alkenylene, each of which is optionally substituted with one or more R^{11b}. In some embodiments, R¹ is —CH₃;

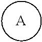

is selected from

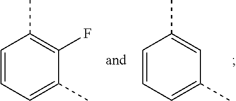

L¹ is C₁₋₂ haloalkylene;

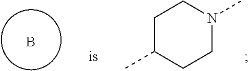

and L² is —(C₁₋₂ alkylene)-C(O)N(CH₃)—(C₃₋₄ alkylene)- or —(C₁₋₂ alkylene)-C(O)NH—(C₃₋₄ alkylene)-, wherein C₁₋₂ alkylene and C₃₋₄ alkylene are each independently optionally substituted with one or more R^{11b}. In some embodiments, R^{1b}, when present, is selected from halogen, —CN, oxo, C₁₋₃ alkyl, C₁₋₃ haloalkyl, (C₁₋₃ alkyl)-OH, and —OH.

In some embodiments, a compound of Formula (I) is a compound of the formula:

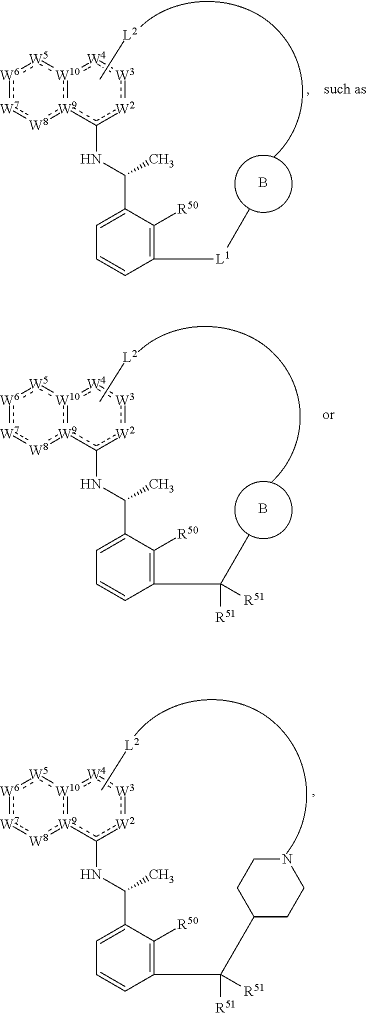

wherein R⁵⁰ is hydrogen or R¹¹ and R⁵¹ is hydrogen or halogen.

In some embodiments, a compound of Formula (I) is a compound of the formula:

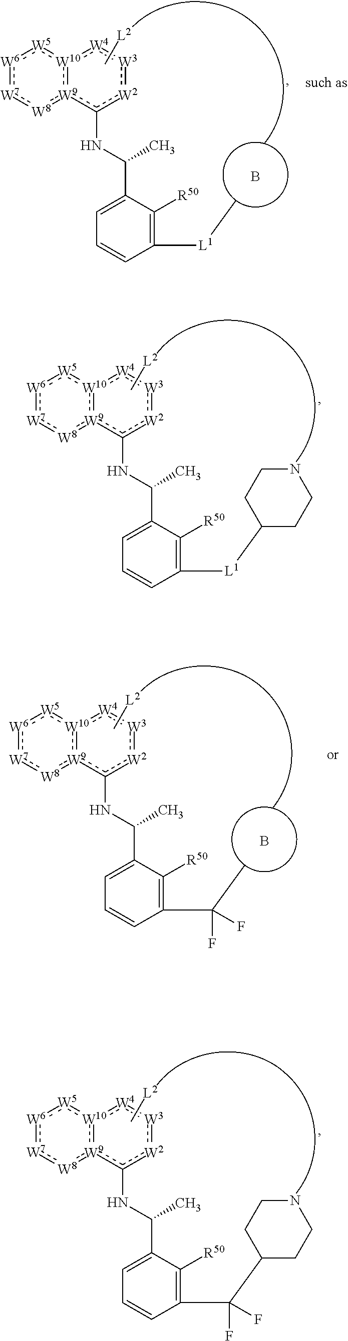

wherein R⁵⁰ is hydrogen or fluoro. In some embodiments, a compound of Formula (I-A) is a compound of the formula:

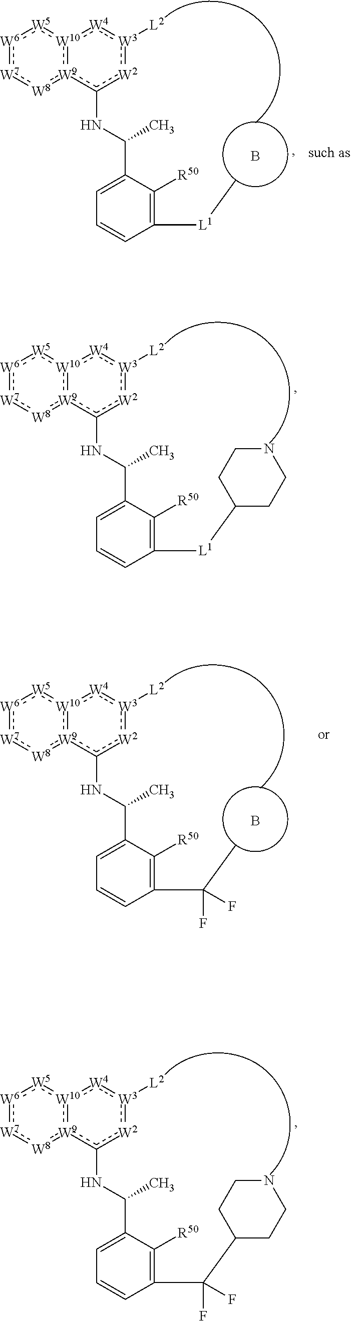

wherein R⁵⁰ is hydrogen or fluoro. In some embodiments, a compound of Formula (I-B) is a compound of the formula:

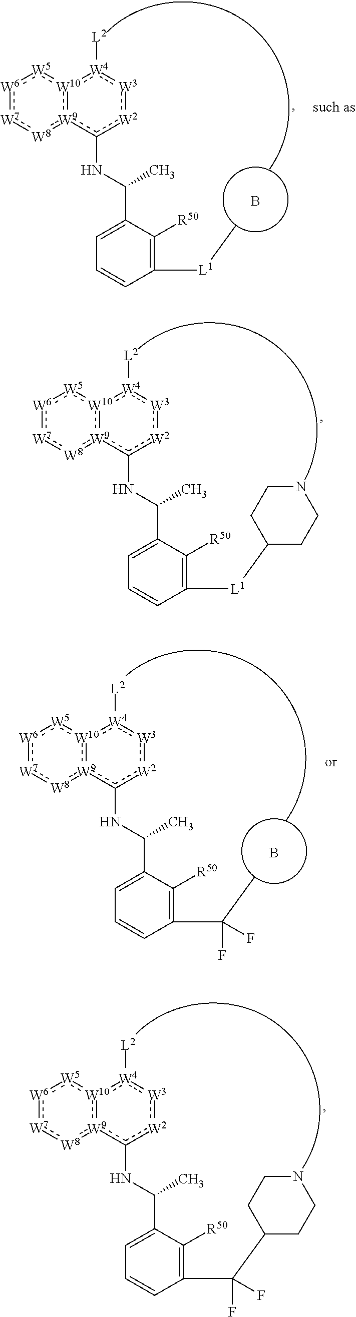

wherein R⁵⁰ is hydrogen or fluoro. In some embodiments, a compound of Formula (I-C) is a compound of the formula:

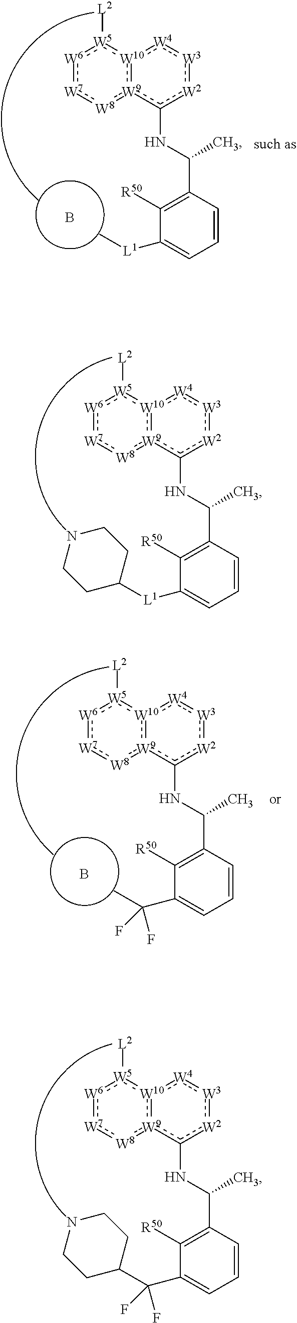

wherein R⁵⁰ is hydrogen or fluoro. In some embodiments, a compound of Formula (I-C) is a compound of the formula,

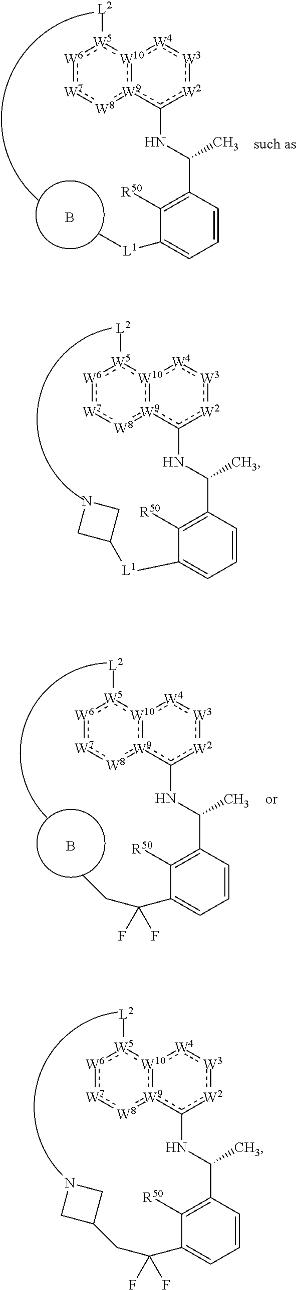

wherein R⁵⁰ is hydrogen or fluoro. In some embodiments, a compound of Formula (I-D) is a compound of the formula:

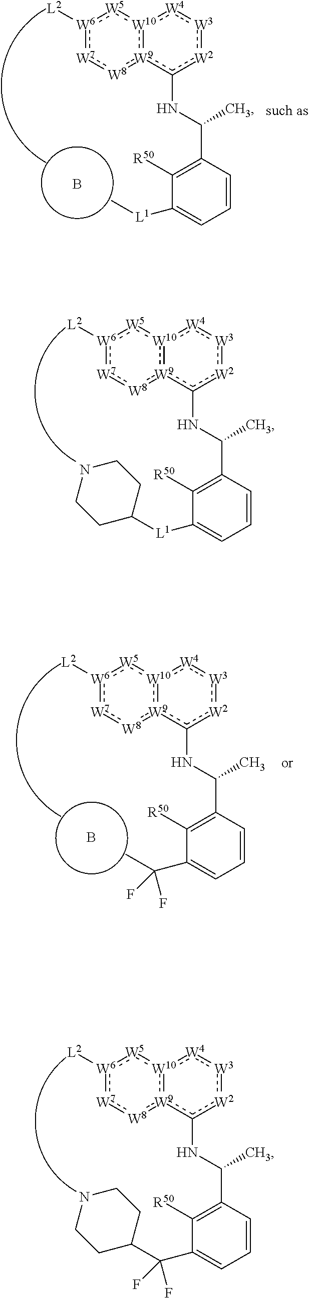

wherein R⁵⁰ is hydrogen or fluoro. In some embodiments, a compound of Formula (I-E) is a compound of the formula:

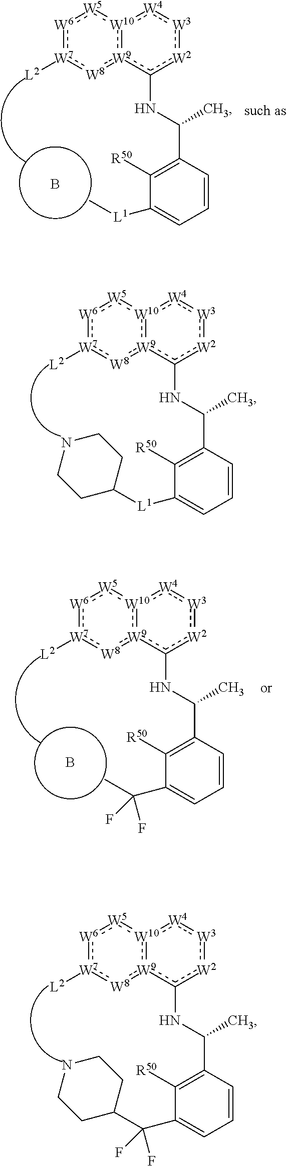

wherein R⁵⁰ is hydrogen or fluoro. In some embodiments, L¹ is C₁₋₃ haloalkylene, such as C₁₋₂ haloalkylene or C₁₋₂ fluoroalkylene. In some embodiments, L² is selected from C₆₋₈ alkylene, C₆₋₈ alkenylene, and 6- to 8-membered heteroalkylene, each of which is optionally substituted with one or more R^{11b}. In some embodiments, L² is selected from C₆₋₈ alkylene and C₆₋₈ alkenylene, each of which is optionally substituted with one or more R^{11b}. In some embodiments, L² is —(C₁₋₂ alkylene)-C(O)N(CH₃)—(C₃₋₄ alkylene)- or —(C₁₋₂ alkylene)-C(O)NH—(C₃₋₄ alkylene)-, wherein C₁₋₂ alkylene and C₃₋₄ alkylene are each independently optionally substituted with one or more R^{11b}. In some embodiments, R^{11b}, when present, is selected from halogen, —CN, C₁₋₃ alkyl, C₁₋₃ haloalkyl, (C₁₋₃ alkyl)-OH, and —OH. In some embodiments, R⁵⁰ is hydrogen. In some embodiments, R⁵⁰ is fluoro.

In certain aspects, the present disclosure provides a compound of Formula (I-C1):

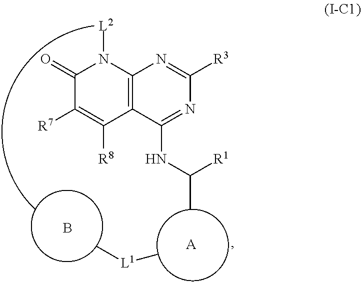

or a pharmaceutically acceptable salt or solvate thereof, wherein:

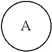

is selected from

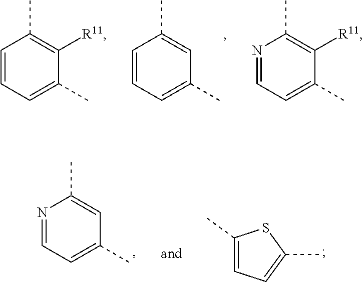

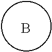

is selected from azetidine, pyrrolidine, and piperidine, each of which is optionally substituted with one or more —CH₃;
L¹ is C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{b};
R¹ is —CH₃;
R³ is hydrogen or —CH₃;
R⁷ is selected from

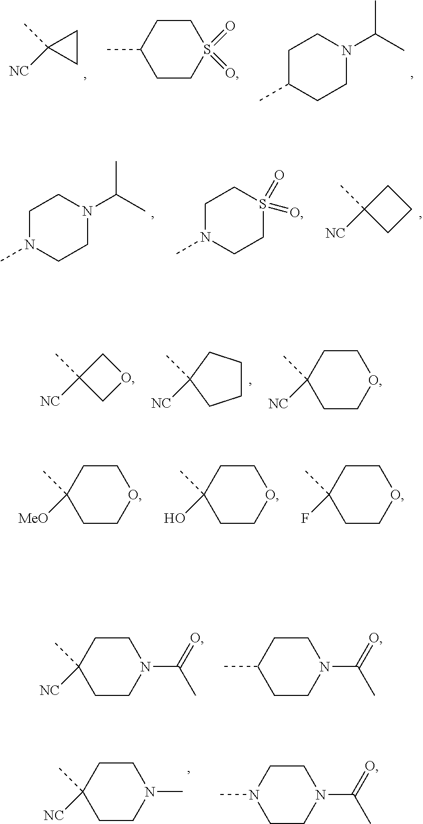

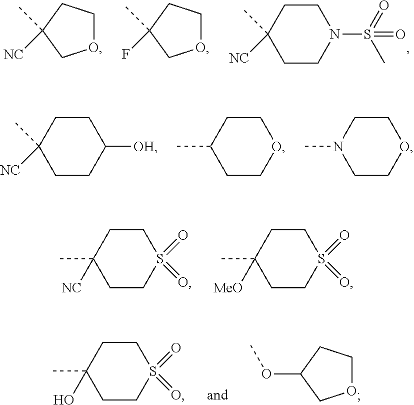

R⁸ is hydrogen;
R¹¹ is selected from fluorine and —CH₃; and
R^{11b} is selected from halogen, oxo, C₁₋₆ alkyl, C₁₋₆ haloalkyl, (C₁₋₆ alkyl)-OH, and —OH.

In certain aspects, the present disclosure provides a compound of Formula (I-C1):

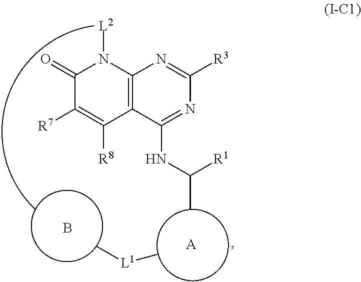

or a pharmaceutically acceptable salt or solvate thereof, wherein:

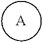

is selected from

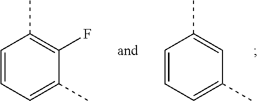

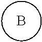

is selected from azetidine, pyrrolidine, and piperidine;
L¹ is selected from —CF₂—, —CF₂CH₂—, and —CF₂CH₂CH₂—;
L² is selected from C₅₋₁₀ alkylene, C₅₋₁₀ alkenylene, 5- to 10-membered heteroalkylene, and 5- to 10-membered heteroalkenylene;
R¹ is —CH₃;
R³ is hydrogen or —CH₃;
R⁷ is selected from

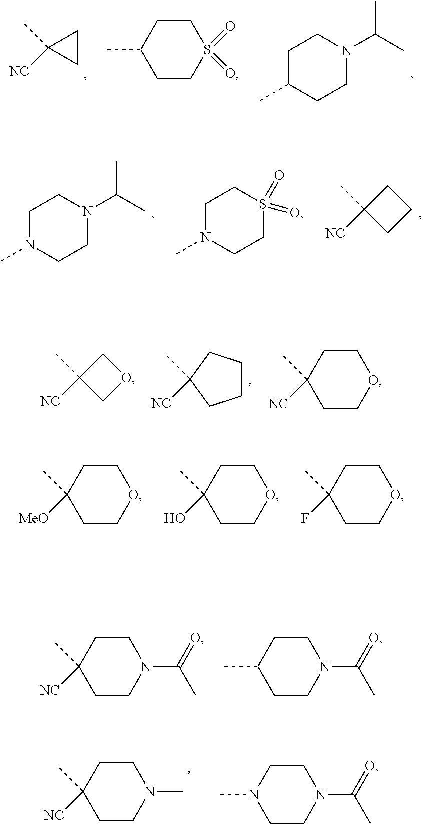

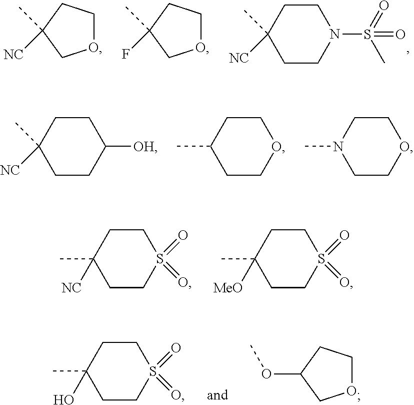

R⁸ is hydrogen.

In certain aspects, the present disclosure provides a compound of Formula (I-C1):

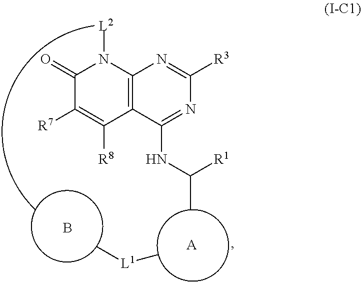

or a pharmaceutically acceptable salt or solvate thereof, wherein:

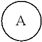

is selected from

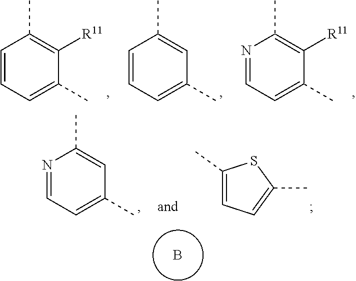

is selected from azetidine, pyrrolidine, and piperidine, each of which is optionally substituted with one or more —CH₃;
L¹ is C₁₋₃ haloalkylene;
L² is C₅₋₁₀ alkenylene optionally substituted with one or more R^{11b};
R¹ is —CH₃;
R³ is —CH₃;
R⁷ is selected from

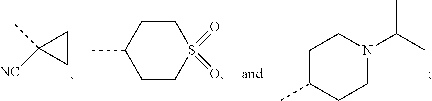

R⁸ is hydrogen;
R¹¹ is selected from fluorine and —CH₃; and
R^{11b} is selected from halogen, oxo, C₁₋₆ alkyl, C₁₋₆ haloalkyl, (C₁₋₆ alkyl)-OH, and —OH.

In certain aspects, the present disclosure provides a compound of Formula (I-C1):

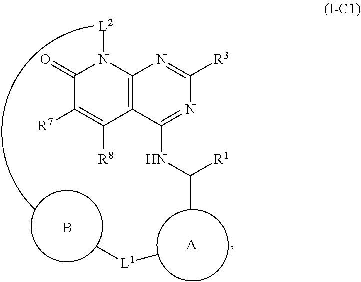

or a pharmaceutically acceptable salt or solvate thereof, wherein:

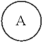

is selected from

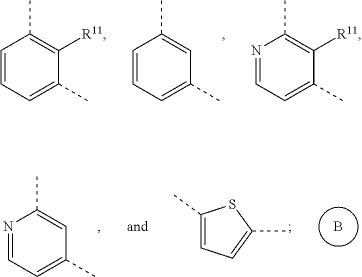

is selected from azetidine, pyrrolidine, and piperidine, each of which is optionally substituted with one or more —CH₃;
L¹ is C₁₋₃ haloalkylene;
L² is selected from 5- to 10-membered heteroalkylene optionally substituted with one or more R^{11b};
R¹ is —CH₃;
R³ is hydrogen or —CH₃;
R⁷ is selected from

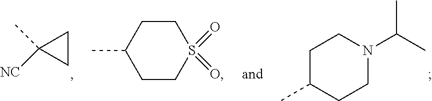

R⁸ is hydrogen;
R¹¹ is selected from fluorine and —CH₃; and
R^{11b} is selected from halogen, oxo, C₁₋₆ alkyl, C₁₋₆ haloalkyl, (C₁₋₆ alkyl)-OH, and —OH.

In certain aspects, the present disclosure provides a compound of Formula (I):

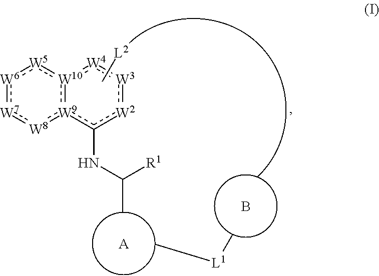

or a pharmaceutically acceptable salt or solvate thereof, wherein:

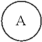

is selected from phenyl and 5- to 7-membered heteroaryl, each of which is optionally substituted with one or more R¹¹;

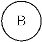

is absent or selected from phenyl and 4- to 8-membered heterocycle, each of which is optionally substituted with one or more R^{11a};
L¹ is selected from a bond and C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}, wherein L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷;
L³ is selected from C₁₋₈ alkylene, C₂₋₈ alkenylene, 2- to 8-membered heteroalkylene, and 3- to 8-membered heteroalkenylene, each of which is optionally substituted with one or more R^{11b};
D is selected from C₃₋₁₀ carbocycle and 3- to 10-membered heterocycle, each of which is optionally substituted with one or more R^{11d};
L⁴ is selected from C₁₋₈ alkylene, C₂₋₈ alkenylene, 2- to 8-membered heteroalkylene, and 3- to 8-membered heteroalkenylene, each of which is optionally substituted with one or more R^{11b};
W² is N;
W³ is selected from N(R^{3b}), N, C(R³), and C(O);
W⁴ is selected from N(R^{4b}), N, C(R⁴), and C(O);
W⁵ is selected from N(R^{5b}), N, and C(R⁵);
W⁶ is selected from C(R⁶) and C(O);
W⁷ is C(R⁷);
W⁸ is C(R⁸);
W⁹ and W¹⁰ are each C;
R¹ is —CH₃;
R², R^{2a}, R^{3a}, R^{4a}, R^{5a}, R^{6a}, R^{7a}, R⁸, and R^{8a} are each independently selected from hydrogen and —CH₃;
R³, R⁴, R⁵, and R⁶ are each independently selected from a bond to L², hydrogen, halogen, —CN, C₁₋₃ alkyl, C₁₋₃ haloalkyl, —OH, —OCH₃, —NH₂, —NHCH₃, —N(CH₃)₂;
R⁷ is selected from a bond to L², C₁₋₆ alkyl, C₃₋₁₀ carbocycle, 3- to 10-membered heterocycle, —OR¹², —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰;
R^{3b}, R^{4b}, and R^{5b} are each independently selected from a bond to L², hydrogen, and C₁₋₃ alkyl;
R¹¹, R^{11a}, and R^{11d} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In certain aspects, the present disclosure provides a compound of Formula (III):

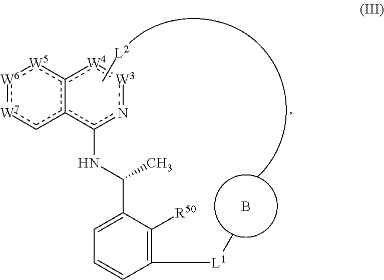

or a pharmaceutically acceptable salt or solvate thereof, wherein:

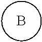

is selected from 3- to 8-membered heterocycle, optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷;
L³ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{1b};
D is absent or selected from C₃₋₁₂ carbocycle and 3- to 12-membered heterocycle, each of which is optionally substituted with one or more R^{11d};
L⁴ is selected from C₁₋₁₀ alkylene, C₂₋₁₀ alkenylene, C₂₋₁₀ alkynylene, 2- to 10-membered heteroalkylene, and 3- to 10-membered heteroalkenylene, each of which is optionally substituted with one or more R^{11b};
W³ is selected from N(R^{3b}) and N and W⁴ is selected from C(R⁴) and C(O); or W³ is selected from C(R³) and C(O), and W⁴ is selected from N(R^{4b}) and N;
W⁵ is selected from N(R^{5b}), N, and C(R⁵);
W⁶ is selected from N, C(R⁶), and C(O);
W⁷ is selected from N, C(R⁷), and C(O);
R⁵⁰ is hydrogen or halogen;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{3b}, R^{4b}, and R^{5b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R^{11a} and R^{11d} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In certain aspects, the present disclosure provides a compound of Formula (III):

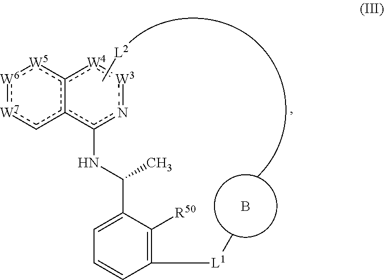

or a pharmaceutically acceptable salt or solvate thereof, wherein:

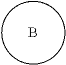

is selected from 3- to 8-membered heterocycle, optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷;
W³ is selected from N(R^{3b}) and N and W⁴ is selected from C(R⁴) and C(O); or W³ is selected from C(R³) and C(O), and W⁴ is selected from N(R^{4b}) and N;
W⁵ is selected from N(R^{5b}), N, and C(R⁵);
W⁶ is selected from N, C(R⁶), and C(O);
W⁷ is selected from N, C(R⁷), and C(O);
R⁵⁰ is hydrogen or halogen;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{3b}, R^{4b}, and R^{5b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R^{11a} is independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In some embodiments, a compound of Formula (III) is a compound of the formula:

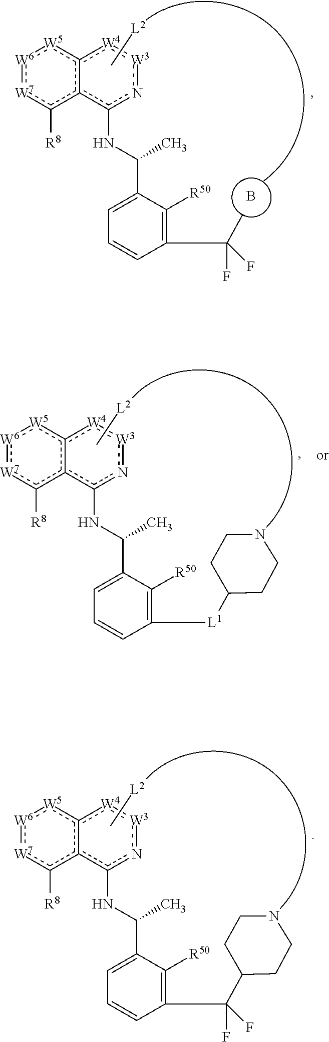

In some embodiments, a compound of Formula (III) is a compound of the formula:

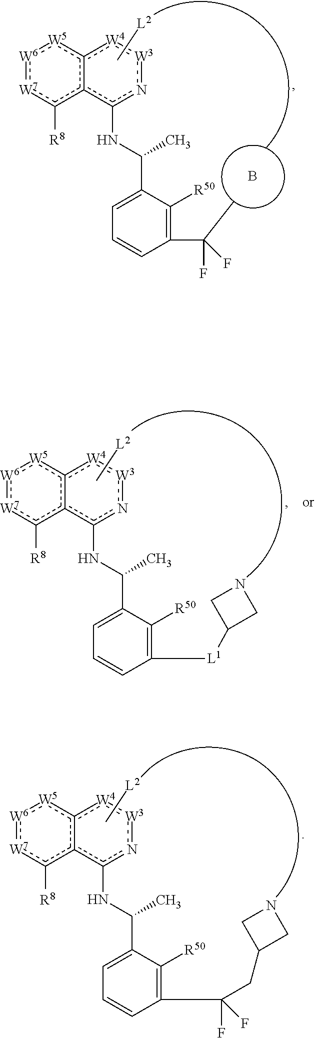

In some embodiments, for a compound of Formula (III),

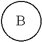

is selected from azetidine, pyrrolidine, and piperidine, each of which is optionally substituted with one or more R^{11a}. In some embodiments,

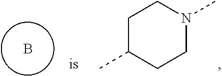

each of which is optionally substituted with one or more R^{11a}. In some embodiments,

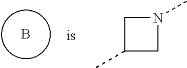

optionally substituted with one or more R¹¹. In some embodiments, L¹ is C₁₋₃ haloalkylene. In some embodiments, L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}, wherein L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷; L³ is selected from C₁₋₈ alkylene, C₂₋₈ alkenylene, 2- to 8-membered heteroalkylene, and 3- to 8-membered heteroalkenylene, each of which is optionally substituted with one or more R^{11b}; D is selected from C₃₋₁₀ carbocycle and 3- to 10-membered heterocycle, each of which is optionally substituted with one or more R^{11d}; and L⁴ is selected from C₁₋₈ alkylene, C₂₋₈ alkenylene, 2- to 8-membered heteroalkylene, and 3- to 8-membered heteroalkenylene, each of which is optionally substituted with one or more R^{11b}.

In certain aspects, the present disclosure provides a compound of the formula:

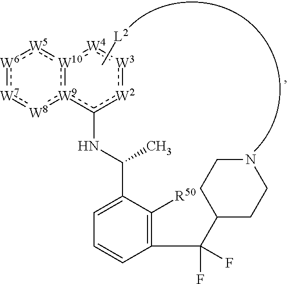

or a pharmaceutically acceptable salt or solvate thereof, wherein:
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}, wherein L² is covalently bound to one of W³, W⁴, W⁵, W⁶, or W⁷; or L² is -L³-D-L⁴-, wherein L⁴ is covalently bound to one of W³, W⁴, W⁵, W⁶, or W⁷;
L³ is selected from C₁₋₈ alkylene, C₂₋₈ alkenylene, 2- to 8-membered heteroalkylene, and 3- to 8-membered heteroalkenylene, each of which is optionally substituted with one or more R^{11b};
D is selected from C₃₋₁₀ carbocycle and 3- to 10-membered heterocycle, each of which is optionally substituted with one or more R^{11d};
L⁴ is selected from C₁₋₈ alkylene, C₂₋₈ alkenylene, 2- to 8-membered heteroalkylene, and 3- to 8-membered heteroalkenylene, each of which is optionally substituted with one or more R^{11b};
W² is selected from N(R^{2b}), N, C(R²), C(R²)(R^{2a}), and C(O);
W³ is selected from N(R^{3b}), N, C(R³), C(R³)(R^{3a}), and C(O);
W⁴ is selected from N(R^{4b}), N, C(R⁴), C(R⁴)(R^{4a}), and C(O);
W⁵ is selected from N(R^{5b}), N, C(R⁵), C(R⁵)(R^{5a}), and C(O);
W⁶ is selected from N(R^{6b}), N, C(R⁶), C(R⁶)(R^{6a}), and C(O);
W⁷ is selected from N(R^{7I}), N, C(R⁷), C(R⁷)(R^{7a}), and C(O);
W⁸ is selected from N(R^{8b}), N, C(R⁸), C(R⁸)(R^{8a}), and C(O);
W⁹ is selected from N, C(R⁹), and C;
W¹⁰ is selected from N, C(R¹⁰), and C;
R², R^{2a}, R^{3a}, R^{4a}, R^{5a}, R^{6a}, R^{7a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{2b} and R^{8b} are each independently selected from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R^{3b}, R^{4b}, R^{5b}, R^{6b}, and R^{7b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R⁹ and R¹⁰ are each independently selected from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein each C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) is independently optionally substituted with one, two, or three R²⁰;
R^{11d} is independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
   [symbol] indicates a single or double bond such that all valences are satisfied.

In certain aspects, the present disclosure provides a compound of Formula (II-B) or (II-C):

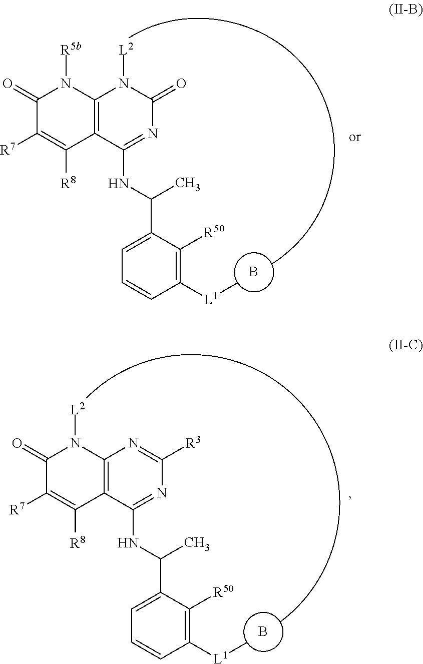

or a pharmaceutically acceptable salt or solvate thereof, wherein:

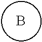

is absent or 4- to 8-membered heterocycle optionally substituted with one or more R^{11a};
L¹ is C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}; or L² is -L³-D-L⁴-;
L³ is selected from C₁₋₈ alkylene, C₂₋₈ alkenylene, 2- to 8-membered heteroalkylene, and 3- to 8-membered heteroalkenylene, each of which is optionally substituted with one or more R^{1b}; D is selected from C₃₋₁₀ carbocycle and 3- to 10-membered heterocycle, each of which is optionally substituted with one or more R^{11d};
L⁴ is selected from C₁₋₈ alkylene, C₂₋₈ alkenylene, 2- to 8-membered heteroalkylene, and 3- to 8-membered heteroalkenylene, each of which is optionally substituted with one or more R^{11b};
R³ and R⁸ are each independently selected from hydrogen and —CH₃;
R⁷ is selected from C₃₋₈ carbocycle and 3- to 8-membered heterocycle, each of which is optionally substituted with one, two, or three R²⁰;
R^{5b} is selected from hydrogen and C₁₋₃ alkyl;
R⁵⁰ is selected from hydrogen and halogen;
R^{11a} and R^{11d} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from hydrogen and C₁₋₆ alkyl;
R⁴ is independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, the present disclosure provides a compound of Formula (II-B) or (II-C):

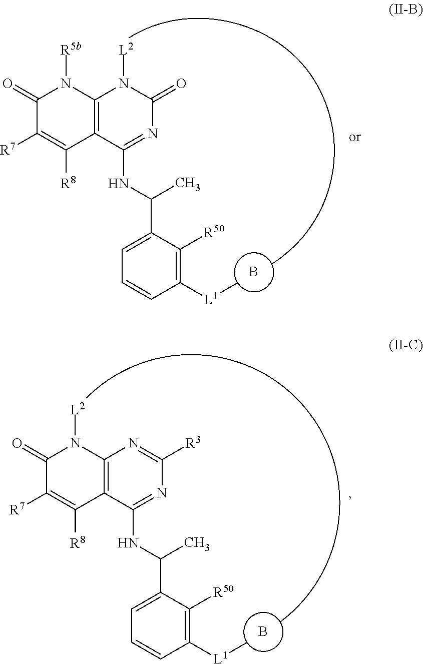

or a pharmaceutically acceptable salt or solvate thereof, wherein:

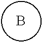

is absent or 4- to 8-membered heterocycle optionally substituted with one or more R^{11a};
L¹ is C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b};
R³, R^{5b}, and R⁸ are each independently selected from hydrogen and —CH₃;
R⁷ is selected from C₃₋₈ carbocycle and 3- to 8-membered heterocycle, each of which is optionally substituted with one, two, or three R²⁰;
R⁵⁰ is selected from hydrogen and halogen;
R^{11a} is independently selected at each occurrence from halogen, C₁₋₃ alkyl, and C₁₋₃ haloalkyl;
R^{11b} is independently selected at each occurrence from halogen, oxo, —CN, C₁₋₃ alkyl, and —OH; and
R²⁰ is independently selected at each occurrence from halogen, oxo, —CN, and C₁₋₆ alkyl.

In some embodiments, the present disclosure provides a compound of Formula (II-C):

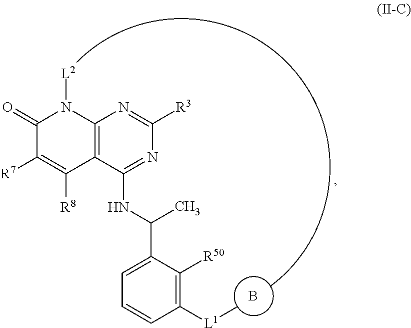

or a pharmaceutically acceptable salt or solvate thereof, wherein:

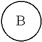

is absent or 4- to 8-membered heterocycle optionally substituted with one or more R^{11a};
L¹ is C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b};
R³ is hydrogen;
R⁸ is selected from hydrogen and —CH₃;
R⁷ is selected from

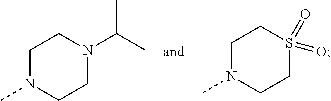

R⁵⁰ is selected from hydrogen and halogen;
R^{11a} is independently selected at each occurrence from halogen, C₁₋₃ alkyl, and C₁₋₃ haloalkyl;
R^{11b} is independently selected at each occurrence from halogen, oxo, —CN, C₁₋₃ alkyl, and —OH; and
R²⁰ is independently selected at each occurrence from halogen, oxo, —CN, and C₁₋₆ alkyl.

In some embodiments, the present disclosure provides a compound of Formula (II-C):

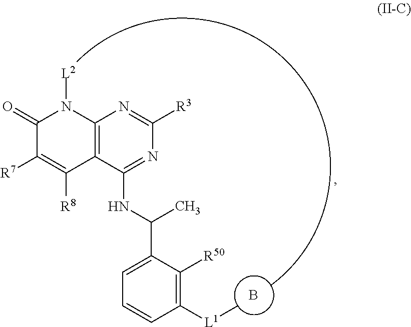

or a pharmaceutically acceptable salt or solvate thereof, wherein:

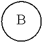

is 4- to 8-membered heterocycle optionally substituted with one or more R^{11a};
L¹ is C₁₋₃ haloalkylene;
L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b};
R³ is selected from hydrogen and —CH₃;
R⁸ is selected from hydrogen and —CH₃;
R⁷ is selected from

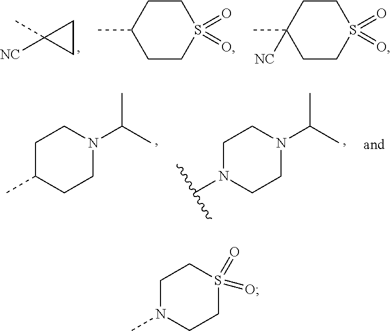

R⁵⁰ is selected from hydrogen and halogen;
R^{11a} is independently selected at each occurrence from halogen, C₁₋₃ alkyl, and C₁₋₃ haloalkyl; and
R^{11b} is independently selected at each occurrence from halogen, oxo, —CN, C₁₋₃ alkyl, and —OH.

In some embodiments, the present disclosure provides a compound of Formula (II-C):

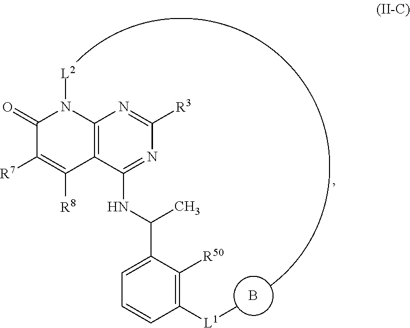

or a pharmaceutically acceptable salt or solvate thereof, wherein:

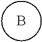

is 4- to 6-membered heterocycle;
L¹ is C₁₋₃ haloalkylene;
L² is selected from C₅₋₁₀ alkylene, C₅₋₁₀ alkenylene, and 5- to 10-membered heteroalkylene;
R³ is selected from hydrogen and —CH₃;
R⁸ is hydrogen;
R⁷ is selected

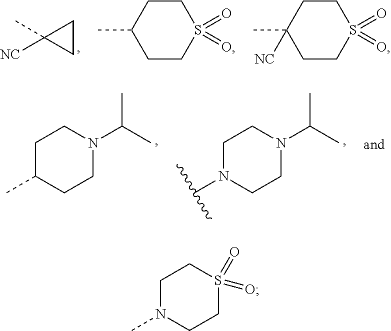

and
R⁵⁰ is selected from hydrogen and halogen.

In some embodiments, the present disclosure provides a compound of Formula (II-C):

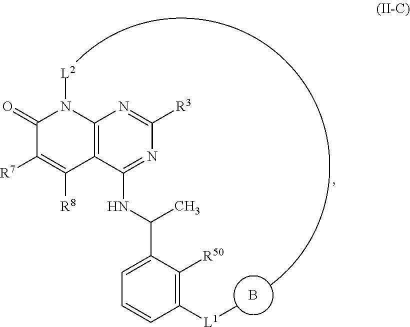

or a pharmaceutically acceptable salt or solvate thereof, wherein:

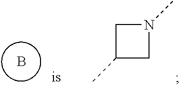

L¹ is —CF₂CH₂—;
L² is 5- to 10-membered heteroalkylene, wherein the heteroalkylene comprises one oxygen atom;
R³ is selected from hydrogen and —CH₃;
R⁸ is hydrogen;
R⁷ is selected from

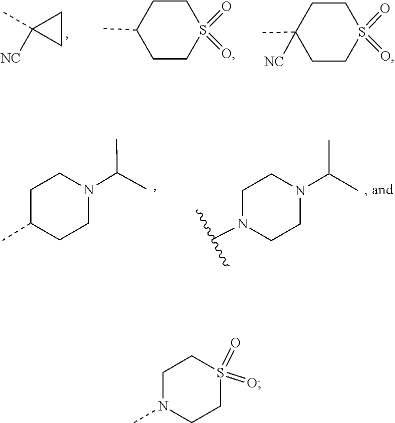

and
R⁵⁰ is selected from hydrogen and halogen.

In some embodiments, the present disclosure provides a compound of Formula (II-C):

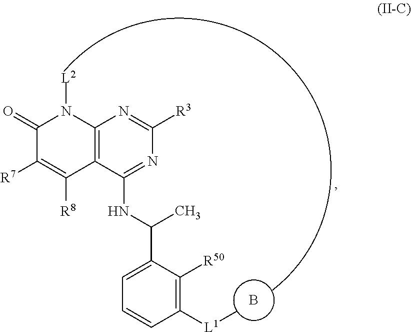

or a pharmaceutically acceptable salt or solvate thereof, wherein:

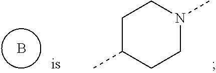

L¹ is —CF₂—;
L² is selected from C₅₋₁₀ alkenylene;
R³ is selected from hydrogen and —CH₃;
R⁸ is hydrogen;
R⁷ is selected from

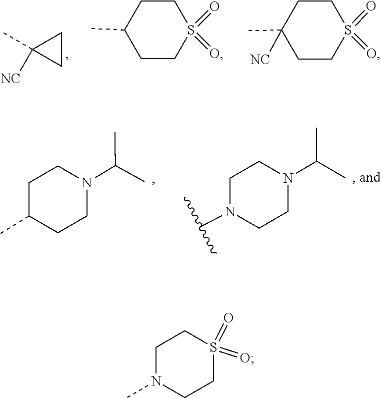

and
R⁵⁰ is selected from hydrogen and halogen.

In some embodiments, a compound of Formula (II-B) is a compound of the formula:

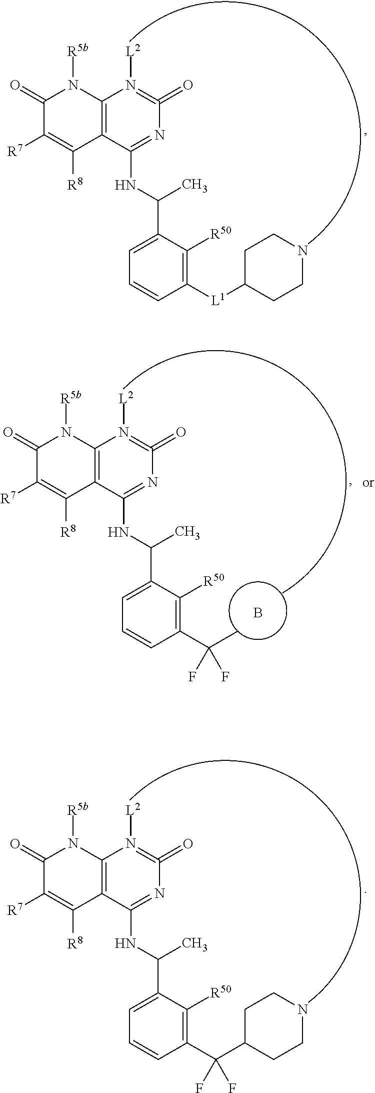

In some embodiments, R^{5b} is —CH₃; R⁸ is hydrogen; and R⁵⁰ is selected from hydrogen and fluoro. In some embodiments, R^{5b} is —CH₃; R⁸ is hydrogen; and R⁵⁰ is hydrogen. In some embodiments, R^{5b} is —CH₃; R⁸ is hydrogen; and R⁵⁰ is fluoro. In some embodiments, R⁷ is selected from

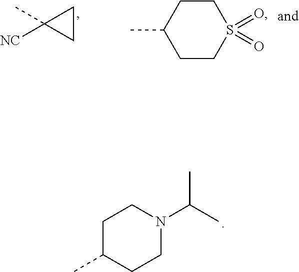

In some embodiments, R^{5b} is —CH₃; R⁸ is hydrogen; R⁵⁰ is hydrogen; and R⁷ is selected from

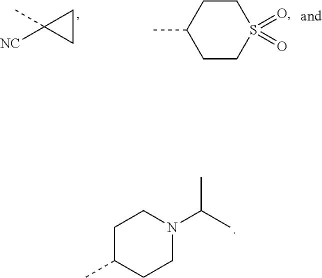

In some embodiments, R^{5b} is —CH₃; R⁸ is hydrogen; R⁵⁰ is fluoro; and R⁷ is selected from

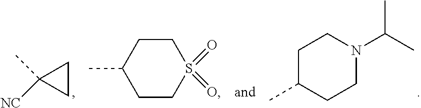

In some embodiments, R^{5b} is —CH₃; R⁸ is hydrogen; R⁵⁰ is selected from hydrogen and fluoro; and R⁷ is

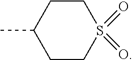

In some embodiments, R^{5b} is —CH₃; R⁸ is hydrogen; R⁵⁰ is selected from hydrogen and fluoro; and R⁷ is

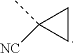

In some embodiments, R^{5b} is —CH₃; R⁸ is hydrogen; R⁵⁰ is selected from hydrogen and fluoro; and R⁷ is

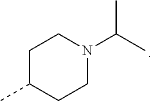

In some embodiments, a compound of Formula (II-C) is a compound of the formula:

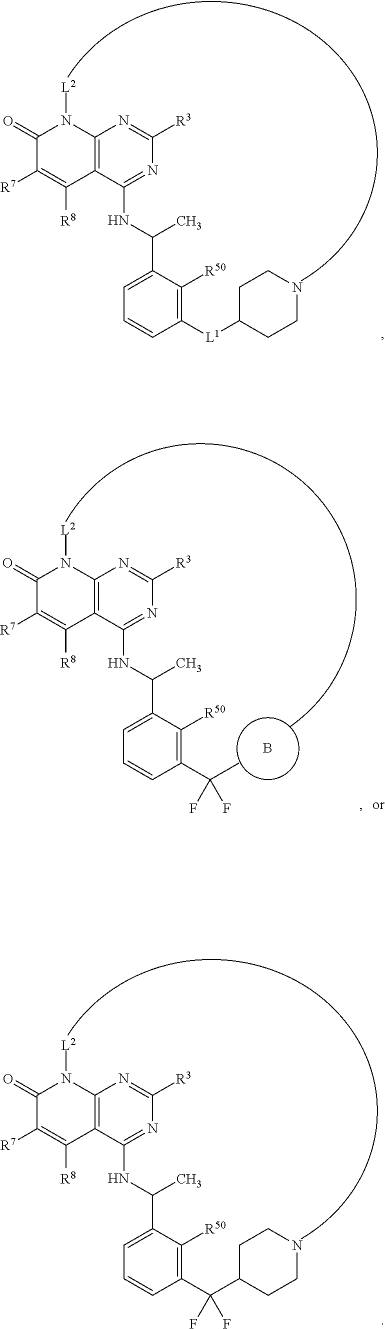

In some embodiments, a compound of Formula (II-C) is a compound of the formula:

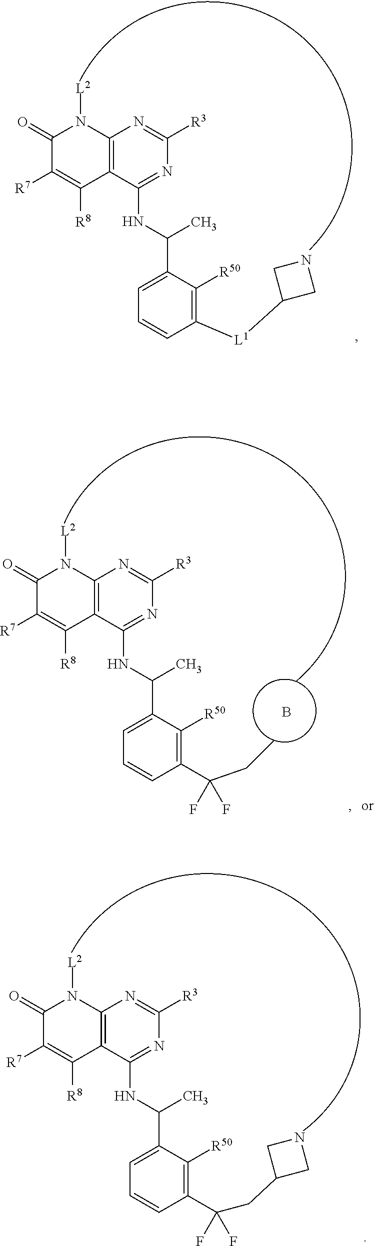

In some embodiments, R³ is selected from hydrogen and —CH₃; R⁸ is hydrogen; and R⁵⁰ is selected from hydrogen and fluoro. In some embodiments, R³ is selected from hydrogen and —CH₃; R⁸ is hydrogen; and R⁵⁰ is hydrogen. In some embodiments, R³ is selected from hydrogen and —CH₃; R⁸ is hydrogen; and R⁵⁰ is fluoro. In some embodiments, R³ is hydrogen; R⁸ is hydrogen; and R⁵⁰ is hydrogen. In some embodiments, R³ is hydrogen; R⁸ is hydrogen; and R⁵⁰ is fluoro. In some embodiments, R³ is —CH₃; R⁸ is hydrogen; and R⁵⁰ is hydrogen. In some embodiments, R³ is —CH₃; R⁸ is hydrogen; and R⁵⁰ is fluoro. In some embodiments, R⁷ is selected from

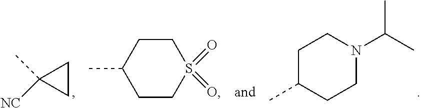

In some embodiments, R³ is selected from hydrogen and —CH₃; R⁸ is hydrogen; R⁵⁰ is hydrogen; and R⁷ is selected from

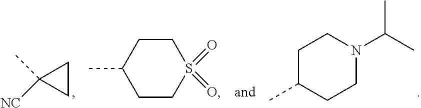

In some embodiments, R³ is selected from hydrogen and —CH₃; R⁸ is hydrogen; R⁵⁰ is fluoro; and R⁷ is selected from

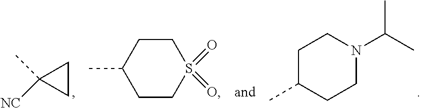

In some embodiments, R³ is hydrogen; R⁸ is hydrogen; R⁵⁰ is selected from hydrogen and fluoro; and R⁷ is selected from

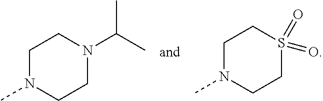

In some embodiments, R³ is hydrogen; R⁸ is hydrogen; R⁵⁰ is hydrogen; and R⁷ is selected from

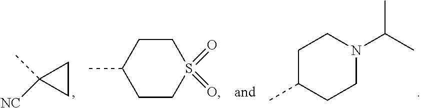

In some embodiments, R³ is hydrogen; R⁸ is hydrogen; R⁵⁰ is fluoro; and R⁷ is selected from

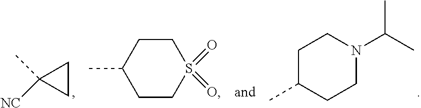

In some embodiments, R³ is —CH₃; R⁸ is hydrogen; R⁵⁰ is hydrogen; and R⁷ is selected from

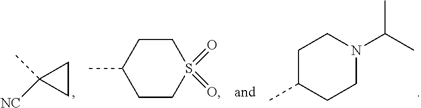

In some embodiments, R³ is —CH₃; R⁸ is hydrogen; R⁵⁰ is fluoro; and R⁷ is selected from

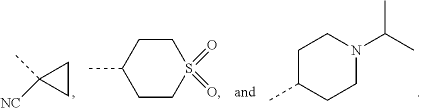

In some embodiments, R³ is selected from hydrogen and —CH₃; R⁸ is hydrogen; R⁵⁰ is selected from hydrogen and fluoro; and R⁷ is

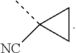

In some embodiments, R³ is selected from hydrogen and —CH₃; R⁸ is hydrogen; R⁵⁰ is selected from hydrogen and fluoro; and R⁷ is

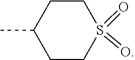

In some embodiments, R³ is selected from hydrogen and —CH₃; R⁸ is hydrogen; R⁵⁰ is selected from hydrogen and fluoro; and R⁷ is

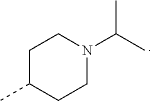

Embodiments disclosed herein that refer to a compound of Formula (I), (I-A), (I-B), (I-B1), (I-B2), (I-C), (I-C1), (I-C2), (I-C3), (I-D), (I-D1), (I-D2), (I-E), and/or (I-E1) are also intended to apply to a compound of Formula (I-1), (II-B), (II-C), and (III) unless the context of the embodiment clearly dictates otherwise (e.g., the embodiment refers solely to a variable not present in the compound of Formula (I-1), (II-B), (II-C), or (III), such as R¹).

In some embodiments, a compound of Formula (I) is a compound of the formula:

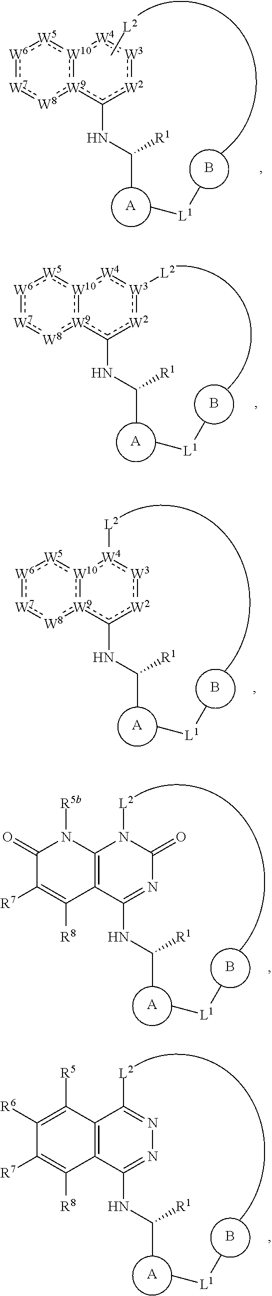

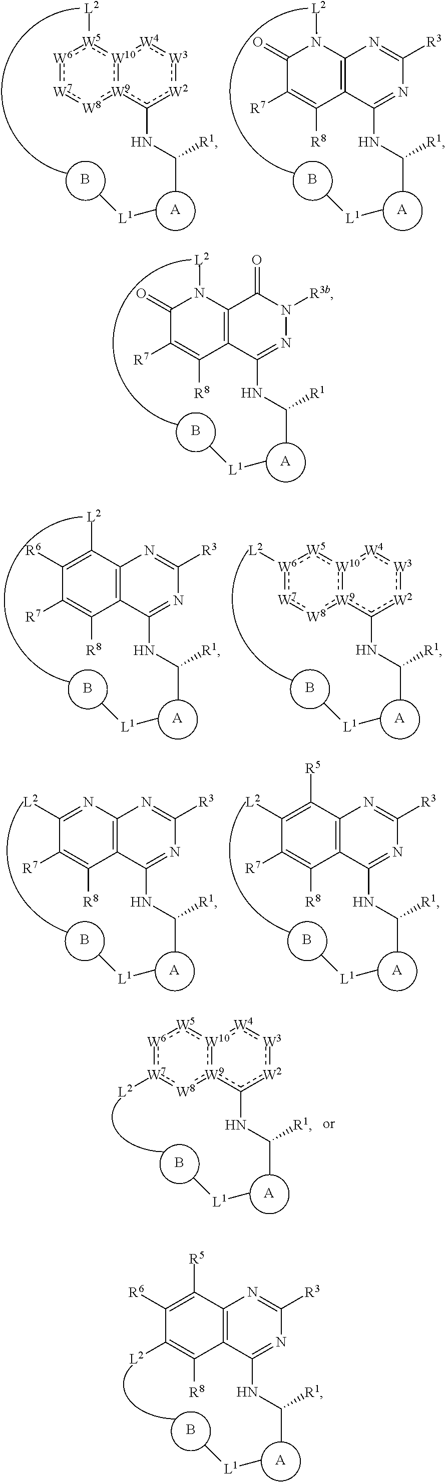

or a salt or solvate thereof.

In certain aspects, the present disclosure provides a compound of Formula (I-1):

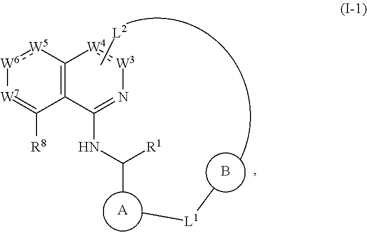

or a pharmaceutically acceptable salt or solvate thereof, wherein:

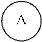

is selected from C₅₋₇ carbocycle and 5- to 7-membered heterocycle, each of which is optionally substituted with one or more R¹¹;

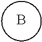

is absent or selected from C₃₋₈ carbocycle and 3- to 8-membered heterocycle, each of which is optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷;
W³ is selected from N(R^{3b}), N, C(R³), and C(O);
W⁴ is selected from N(R^{4b}), N, C(R⁴), and C(O);
W⁵ is selected from N(R^{5b}), N, and C(R⁵);
W⁶ is selected from C(R⁶) and C(O);
W⁷ is C(R⁷);
R¹ is C₁₋₃ alkyl optionally substituted with one or more R^{11c};
R⁸ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{3b}, R^{4b}, and R^{5b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R¹¹ and R^{11a} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11c} is independently selected at each occurrence from halogen, —OR¹², and —N(R¹²)(R¹³);
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or R¹⁷ and R^{17a}, together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and
[symbol] indicates a single or double bond such that all valences are satisfied.

In some embodiments, the compound of Formula (I-1) is a compound of Formula (I-B1) or (I-B2):

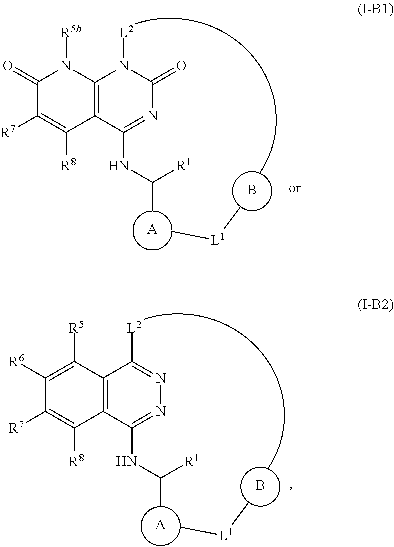

or a pharmaceutically acceptable salt or solvate thereof.

In some embodiments, the compound of Formula (I-1) is a compound of Formula (I-C1), (I-C2), or (I-C3):

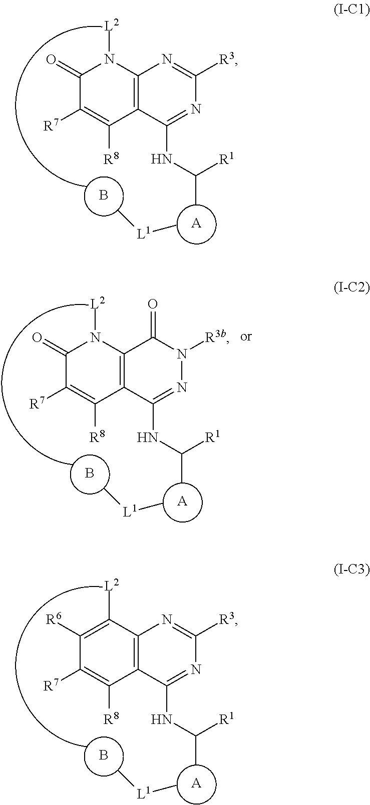

or a pharmaceutically acceptable salt or solvate thereof.

In some embodiments, for a compound of Formula (I-1), (I-B1), (I-B2), (I-C1), (I-C2), or (I-C3), R¹ is —CH₃. In some embodiments, for a compound of Formula (I-1), (I-C1), or (I-C3), R³ is selected from hydrogen, —CN, —OR¹², and —CH₃. In some embodiments, for a compound of Formula (I-1), (I-B2), or (I-C3), R⁶ is selected from hydrogen, —OR¹², and C₁₋₆ alkyl optionally substituted with one, two, or three R²⁰, and wherein R¹² is selected from C₁₋₆ alkyl. In some embodiments, for a compound of Formula (I-1), (I-B1), (I-B2), (I-C1), (I-C2), or (I-C3), R⁸ is hydrogen.

In some embodiments, for a compound of Formula (I-1), (I-B1), (I-B2), (I-C1), (I-C2), or (I-C3), R⁷ is selected from C₁₋₆ alkyl, C₃₋₁₀ cycloalkyl, 3- to 10-membered heterocycloalkyl, and —N(R¹²)(R¹³), wherein C₁₋₆ alkyl, C₃₋₁₀ cycloalkyl, and 3- to 10-membered heterocycloalkyl are optionally substituted with one, two, or three R²⁰. In some embodiments, R⁷ is selected from

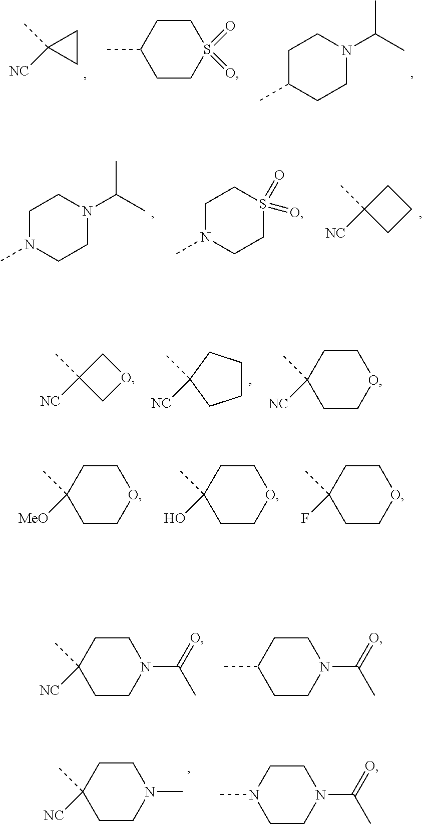

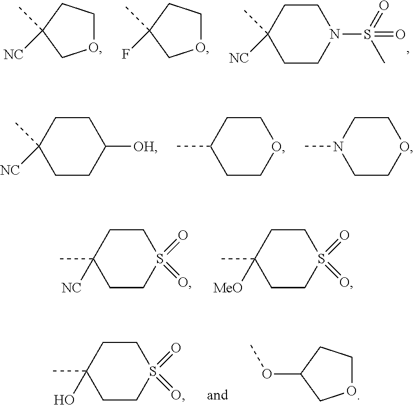

In some embodiments, R⁷ is selected from

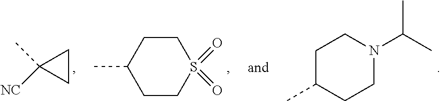

In some embodiments, for a compound of Formula (I-1), (I-B1), (I-B2), (I-C1), (I-C2), or (I-C3),

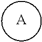

is selected from phenyl and 5- to 7-membered heteroaryl, each of which is optionally substituted with one or more R¹¹. In some embodiments,

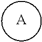

is selected from

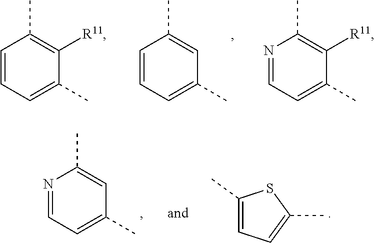

In some embodiments, R¹¹ is independently selected from fluorine and —CH₃.

In some embodiments, for a compound of Formula (I-1), (I-B1), (I-B2), (I-C1), (I-C2), or (I-C3), L¹ is C₁₋₃ haloalkylene. In some embodiments, L¹ is selected from —CF₂—, —CF₂CH₂—, and —CF₂CH₂CH₂—.

In some embodiments, for a compound of Formula (I-1), (I-B1), (I-B2), (I-C1), (I-C2), or (I-C3),

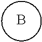

is selected from absent, phenyl, and 4- to 8-membered heterocycle, wherein the phenyl and 4- to 8-membered heterocycle are optionally substituted with one or more R^{11a}. In some embodiments,

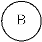

is selected from azetidine, pyrrolidine, and piperidine, each of which is optionally substituted with one or more —CH₃.

In some embodiments, for a compound of Formula (I-1), (I-B1), (I-B2), (I-C1), (I-C2), or (I-C3), L² is selected from C₆₋₁₅ alkylene, C₆₋₁₅ alkenylene, C₆₋₁₅ alkynylene, 6- to 15-membered heteroalkylene, and 6- to 15-membered heteroalkenylene, each of which is optionally substituted with one or more R^{11b}. In some embodiments, L² is selected from C₅₋₁₀ alkylene, C₅₋₁₀ alkenylene, 5- to 10-membered heteroalkylene, and 5- to 10-membered heteroalkenylene, each of which is optionally substituted with one or more R^{11b}. In some embodiments, the alkenylene and heteroalkenylene contain one carbon-carbon double bond. In some embodiments, the heteroalkylene and heteroalkenylene comprise at least one oxygen or nitrogen atom.

In some embodiments, for a compound of Formula (I-1), (I-B1), or (I-B2), R⁵ is hydrogen; R^{5b} is —CH₃; R⁶ is selected from hydrogen and —OCH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen.

In some embodiments, for a compound of Formula (I-1), (I-C1), (I-C2), or (I-C3), R³ is hydrogen or —CH₃; R^{3b} is —CH₃; R⁶ is selected from hydrogen and —OCH₃; R⁷ is selected from C₁₋₆ alkyl, C₃₋₁₀ carbocycle, 3- to 10-membered heterocycle, —N(R¹²)(R¹³), —C(O)R¹⁵, —C(O)N(R¹²)(R¹³), —S(O)₂R¹⁵, and —SO₂N(R¹²)(R¹³), wherein C₁₋₆ alkyl, C₃₋₁₀ carbocycle, and 3- to 10-membered heterocycle are optionally substituted with one, two, or three R²⁰; and R⁸ is hydrogen.

In certain aspects, the present disclosure provides a compound of Formula (A):

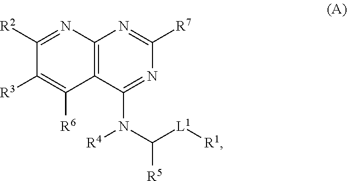

or a pharmaceutically acceptable salt or solvate thereof, wherein:
R¹ is a 3-12 membered cycloalkyl ring, 3-12 membered heterocycloalkyl ring, 6-10 membered aryl ring, or 5-10 membered heteroaryl ring, wherein the 3-12 membered cycloalkyl ring, 3-12 membered heterocycloalkyl ring, 6-10 membered aryl ring, and 5-10 membered heteroaryl ring are optionally substituted with one or more R¹⁰;
L¹ is a bond or C₁₋₆alkyl;
R² is —OR^{2a}, —NR^{2b}R^{2c}, —SR^{2g}, —S(O)R^{2h}, —S(O)₂^{2h}, —S(O)₂R^{2b}R^{2c}, —C(R^{2d})(R^{2e})(R^{2f}), C(O)NR^{2b}R^{2c}, —CN, or halogen;
R² is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —S(O)₂R¹⁵, —S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2b} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —S(O)₂R¹⁵, and —S(O)₂N(R¹²)(R¹³), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2c} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₉heteroaryl are optionally substituted with one, two, or three R^{20a}; or R^{2b} and R^{2c}, together with the nitrogen to which they are attached, form a C₂₋₉heterocycloalkyl ring optionally substituted with one, two, or three R^{20a};
R^{2d} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R⁷)(R^{17a}), wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2e} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2f} is selected from hydrogen, C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2g} is selected from hydrogen, C₂₋₆alkyl, C₂₋₆alkenyl, C₂₋₆alkynyl, C₃₋₁₀cycloalkyl, C₂₋₉heterocycloalkyl, C₆₋₁₀aryl, C₁₋₉ heteroaryl, —C(O)OR¹², —C(O)R¹⁵, —C(O)N(R¹²)(R¹³), —S(O)₂R¹⁵, —S(O)₂N(R¹²)(R¹³), wherein C₂₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R^{2h} is selected from C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₁₀cycloalkyl, C₂₋₉heterocycloalkyl, C₆₋₁₀aryl, and C₁₋₉heteroaryl are optionally substituted with one, two, or three R^{20a};
R³ is selected from halogen, C₁₋₆alkyl, C₂₋₆alkenyl, C₂₋₆alkynyl, C₃₋₁₄cycloalkyl, C₂₋₉heterocycloalkyl, C₆₋₁₀aryl, C₁₋₉ heteroaryl, —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, N(R¹⁴)S(O)R¹⁵, —C(O)R¹⁵, —S(O)R¹⁵, —OC(O)R¹⁵, —C(O)N(R¹²)(R^{13a}), —C(O)C(O)N(R¹²)(R¹³), —N(R¹⁴)C(O)R¹⁵, —S(O)₂R¹⁵, —S(O)₂N(R¹²)(R¹³), —S(O)N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), CH₂S(O) R¹⁵, —CH₂S(O)N(R¹²)(R¹³), —CH₂N(R¹²)S(O)(R¹³) and —P(O)(R¹⁷)(R^{17a}), wherein C₁₋₆alkyl, C₂₋₆alkenyl, C₂₋₆alkynyl, C₃₋₁₄cycloalkyl, C₂₋₉heterocycloalkyl, C₆₋₁₀aryl, and C₁₋₉heteroaryl are optionally substituted with one, two, or three R^{20b};
R⁴ is selected from hydrogen, C₁₋₆alkyl, and C₁₋₆haloalkyl;
R⁵ is selected from hydrogen, C₁₋₆alkyl, and C₁₋₆haloalkyl;
R⁶ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆alkyl, C₂₋₆alkenyl, C₂₋₆alkynyl, C₃₋₁₀cycloalkyl, and C₂₋₉heterocycloalkyl are optionally substituted with one, two, or three R^{20c};
R⁷ is selected from hydrogen, halogen, —CN, C₁₋₆alkyl, C₂₋₆alkenyl, C₂₋₆alkynyl, C₃₋₁₀cycloalkyl, C₂₋₉heterocycloalkyl, C₁₋₉ heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆alkyl, C₂₋₆alkenyl, C₂₋₆alkynyl, C₃₋₁₀cycloalkyl, C₂₋₉heterocycloalkyl, and C₁₋₉heteroaryl are optionally substituted with one, two, or three R^{20c};
each R¹⁰ is independently selected from halogen, —CN, C₁₋₆alkyl, C₂₋₆alkenyl, C₂₋₆alkynyl, C₃₋₇cycloalkyl, C₂₋₉heterocycloalkyl, C₆₋₁₀aryl, C₁₋₉heteroaryl, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉heteroaryl are optionally substituted with one, two, or three R²¹;
each R¹² is independently selected from hydrogen, C₁₋₆alkyl, C₁₋₆ haloalkyl, C₂₋₆alkenyl, C₂₋₆alkynyl, C₃₋₇cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉heteroaryl are optionally substituted with one, two, or three R^{20e};
each R¹³ is independently selected from hydrogen, C₁₋₆alkyl, and C₁₋₆haloalkyl; or R¹² and R¹³, together with the nitrogen to which they are attached, form a C₂₋₉heterocycloalkyl ring optionally substituted with one, two, or three R^{20f},
R^{13a} is selected from C₁₋₆alkyl and C₁₋₆haloalkyl; or R¹² and R^{13a}, together with the nitrogen to which they are attached, form a C₂₋₉heterocycloalkyl ring optionally substituted with one, two, or three R^{20f};
each R¹⁴ is independently selected from hydrogen, C₁₋₆alkyl, and C₁₋₆haloalkyl;
each R¹⁵ is independently selected C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉heteroaryl are optionally substituted with one, two, or three R^{20g};
each R¹⁷ and each R^{17a} are each independently selected from C₁₋₆alkyl and C₃₋₆cycloalkyl, wherein C₁₋₆alkyl and C₃₋₆cycloalkyl are optionally substituted with one, two or three of R^{20h}; or R¹⁷ and R^{17a} are combined to form a C₂₋₉heterocycloalkyl ring;
each R^{20a}, R^{20b}, R^{20c}, R^{20d}, R^{20e}, R^{20f}, R^{20g}, and R^{20h} are each independently selected from halogen, oxo, ═NH, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, ═NH, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₇cycloalkyl, C₂₋₉heterocycloalkyl, C₆₋₁₀aryl, and C₁₋₉heteroaryl, wherein C₃₋₇cycloalkyl, C₂₋₉heterocycloalkyl, C₆₋₁₀aryl, and C₁₋₉heteroaryl are optionally substituted with one, two, or three groups independently selected from halogen and C₁₋₆alkyl;
each R²² is independently selected from H, C₁₋₆alkyl, C₁₋₆haloalkyl, C₂₋₆alkenyl, C₂₋₆alkynyl, C₃₋₇cycloalkyl, C₂₋₉heterocycloalkyl, C₆₋₁₀aryl, and C₁₋₉heteroaryl, wherein C₃₋₇cycloalkyl, C₂₋₉heterocycloalkyl, C₆₋₁₀aryl, and C₁₋₉heteroaryl are optionally substituted with one, two, or three groups independently selected from halogen and C₁₋₆alkyl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉ heteroaryl, wherein C₁₋₆alkyl, C₃₋₇cycloalkyl, C₂₋₉heterocycloalkyl, C₆₋₁₀aryl, and C₁₋₉heteroaryl are optionally substituted with one, two, or three groups independently selected from halogen, C₁₋₆alkyl, C₁₋₆haloalkyl, C₁₋₆alkoxy, C₃₋₇cycloalkyl, C₂₋₉heterocycloalkyl, C₆₋₁₀aryl, and C₁₋₉heteroaryl.

In certain aspects, the present disclosure provides a compound of Formula (A-1):

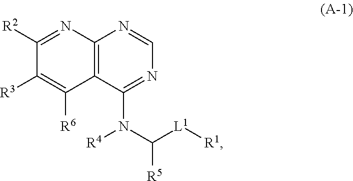

or a pharmaceutically acceptable salt or solvate thereof, wherein:
R¹ is a 6-10 membered aryl ring optionally substituted with one or more R¹⁰;
L¹ is a bond;
R² is —OR²a or halogen;
R^{2a} is selected from C₁₋₆alkyl, C₃₋₁₀cycloalkyl, C₂₋₉heterocycloalkyl, C₆₋₁₀aryl, C₁₋₉heteroaryl, wherein C₁₋₆alkyl, C₃₋₁₀cycloalkyl, C₂₋₉heterocycloalkyl, C₆₋₁₀aryl, and C₁₋₉heteroaryl are optionally substituted with one, two, or three R^{20a};
R³ is selected from halogen, C₁₋₆alkyl, C₃₋₁₄cycloalkyl, C₂₋₉heterocycloalkyl, C₆₋₁₀aryl, and C₁₋₉heteroaryl, wherein C₁₋₆alkyl, C₃₋₁₄cycloalkyl, C₂₋₉heterocycloalkyl, C₆₋₁₀aryl, and C₁₋₉heteroaryl are optionally substituted with one, two, or three R^{20b};
R⁴ is selected from hydrogen, C₁₋₆alkyl, and C₁₋₆haloalkyl;
R⁵ is selected from hydrogen, C₁₋₆alkyl, and C₁₋₆haloalkyl;
R⁶ is selected from hydrogen, halogen, —CN, and C₁₋₆alkyl, wherein C₁₋₆alkyl is optionally substituted with one, two, or three R^{20c};
each R¹⁰ is independently selected from halogen, —CN, C₁₋₆alkyl, C₃₋₇cycloalkyl, C₂₋₉heterocycloalkyl, C₆₋₁₀aryl, C₁₋₉ heteroaryl, wherein C₁₋₆alkyl, C₃₋₇cycloalkyl, C₂₋₉heterocycloalkyl, C₆₋₁₀aryl, and C₁₋₉heteroaryl are optionally substituted with one, two, or three R^{20d};
each R^{20a}, R^{20b}, R^{20c}, and R^{20d} are each independently selected from halogen, oxo, ═NH, —CN, C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, C₁₋₉ heteroaryl, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆alkyl, C₂₋₆alkenyl, C₂₋₆alkynyl, C₃₋₁₀cycloalkyl, —CH₂—C₃₋₁₀cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, ═NH, —CN, C₁₋₆alkyl, C₁₋₆haloalkyl, C₁₋₆alkoxy, C₁₋₆haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
each R²¹ is independently selected from H, C₁₋₆alkyl, C₁₋₆haloalkyl, C₂₋₆alkenyl, C₂₋₆alkynyl, C₃₋₇cycloalkyl, C₂₋₉heterocycloalkyl, C₆₋₁₀aryl, and C₁₋₉heteroaryl, wherein C₃₋₇cycloalkyl, C₂₋₉heterocycloalkyl, C₆₋₁₀aryl, and C₁₋₉heteroaryl are optionally substituted with one, two, or three groups independently selected from halogen and C₁₋₆alkyl;
each R²² is independently selected from H, C₁₋₆alkyl, C₁₋₆haloalkyl, C₂₋₆alkenyl, C₂₋₆alkynyl, C₃₋₇cycloalkyl, C₂₋₉heterocycloalkyl, C₆₋₁₀aryl, and C₁₋₉heteroaryl, wherein C₃₋₇cycloalkyl, C₂₋₉heterocycloalkyl, C₆₋₁₀aryl, and C₁₋₉heteroaryl are optionally substituted with one, two, or three groups independently selected from halogen and C₁₋₆alkyl;
each R²³ is independently selected from H and C₁₋₆alkyl;
each R²⁴ is independently selected from H and C₁₋₆alkyl; and
each R²⁵ is selected from C₁₋₆alkyl, C₂₋₆alkenyl, C₂₋₆alkynyl, C₃₋₇cycloalkyl, C₂₋₉heterocycloalkyl, C₆₋₁₀aryl, and C₁₋₉ heteroaryl, wherein C₁₋₆alkyl, C₃₋₇cycloalkyl, C₂₋₉heterocycloalkyl, C₆₋₁₀aryl, and C₁₋₉heteroaryl are optionally substituted with one, two, or three groups independently selected from halogen, C₁₋₆alkyl, C₁₋₆haloalkyl, C₁₋₆alkoxy, C₃₋₇cycloalkyl, C₂₋₉heterocycloalkyl, C₆₋₁₀aryl, and C₁₋₉heteroaryl.

In some embodiments, the compound of Formula (A-1) is a compound of Formula (Aa-1):

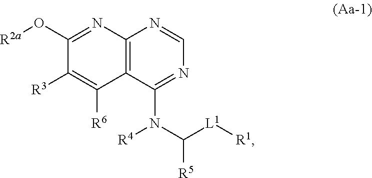

or a pharmaceutically acceptable salt or solvate thereof.

In some embodiments, for a compound of Formula (A), (A-1), or (Aa-1), R^{2a} is C₁₋₆alkyl optionally substituted with one, two, or three R^{20a}. In some embodiments, R^{2a} is unsubstituted C₁₋₆alkyl. In some embodiments, R^{2a} is —CH₃.

In some embodiments, for a compound of Formula (A), (A-1), or (Aa-1), R³ is C₂₋₉heterocycloalkyl optionally substituted with one, two, or three R^{20b}. In some embodiments, R³ is C₃₋₁₀cycloalkyl optionally substituted with one, two, or three R^{20b}. In some embodiments, R³ is C₃₋₄cycloalkyl optionally substituted with one R^{20b}. In some embodiments, R^{20b} is —CN or halogen. In some embodiments, R³ is

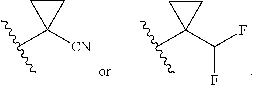 [SMILES: *C1(C#N)CC1 ; *C1(C(F)F)CC1]

In some embodiments, for a compound of Formula (A), (A-1), or (Aa-1), R⁶ is selected from hydrogen, halogen, and unsubstituted C₁₋₆alkyl. In some embodiments, R⁶ is hydrogen. In some embodiments, R⁵ is C₁₋₆alkyl. In some embodiments, R⁵ is —CH₃. In some embodiments, R⁴ is hydrogen.

In some embodiments, for a compound of Formula (A), (A-1), or (Aa-1), R¹ is phenyl substituted with one or more R¹⁰. In some embodiments, R¹⁰ is independently selected from halogen and C₁₋₆alkyl, wherein C₁₋₆alkyl is optionally substituted with one, two, or three R^{20d}. In some embodiments, each R¹⁰ is independently selected from halogen and C₁₋₆alkyl, wherein C₁₋₆alkyl is substituted with one two, or three R^{20d}, and each R^{20d} is halogen or —OH. In some embodiments, R¹ is independently selected from

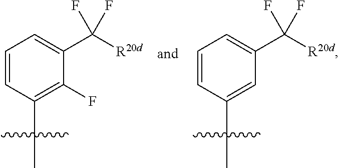

In some embodiments, R^{20d} is C₁₋₆alkyl optionally substituted with one, two, or three groups independently selected from F and —OH. In some embodiments, R^{20d} is C₁₋₃alkyl substituted with one —OH. In some embodiments, R¹ is selected from

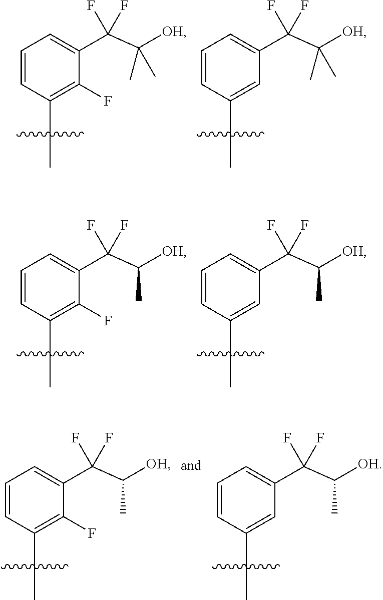

Small molecule SOS1 inhibitors suitable for use in the subject methods—including the synergistic inhibition of growth of a Ph+ cell, such as a CML cell line, in combination with a tyrosine kinase inhibitor against BCR-ABL tyrosine kinase (TKI)—include compounds of Formula (I); Formula (I-A); Formula (I-B); Formula (I-1), encompassing Compound A and Compound B; Formula (I-B1); Formula (I-B2); Formula (I-C); Formula (I-C1); Formula (I-C2); Formula (I-C3); Formula (I-D); Formula (I-D1); Formula (I-D2); Formula (I-E); Formula (I-E1); Formula (II-B); Formula (II-C); Formula (III); Formula (A); Formula (A-1); and Formula (Aa-1). Exemplary small molecule SOS1 inhibitors include, but are not limited to, compounds selected from Table 1 (including Compound A and Compound B), Table 2, or a salt or solvate thereof.

In some embodiments, a compound disclosed herein, such as a compound of Formula (I), (I-A), (I-B), (I-1), (I-B1), (I-B2), (I-C), (I-C1), (I-C2), (I-C3), (I-D), (I-D1), (I-D2), (I-E), (I-E1), (II-B), (II-C), (III), (A), (A-1), or (Aa-1), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1-16, the steps in some cases may be performed in a different order than the order shown in Schemes 1-16. Various modifications to these synthetic reaction schemes may be made and will be suggested to one skilled in the art having referred to the present disclosure. Numberings or R groups in each scheme typically have the same meanings as those defined elsewhere herein unless otherwise indicated.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

In general, compounds of the disclosure may be prepared by the following reaction schemes:

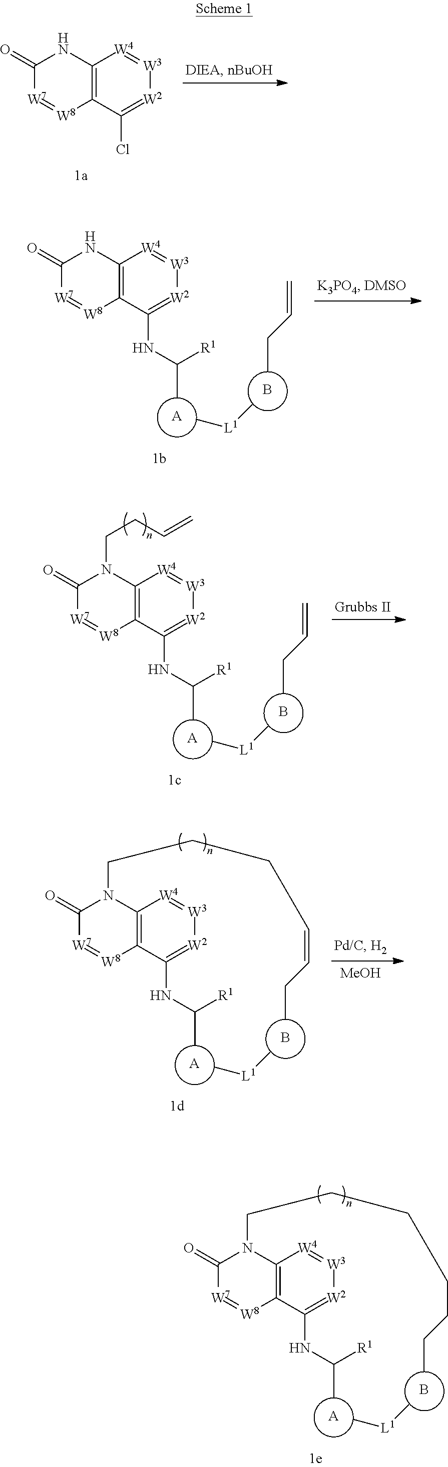

In some embodiments, a compound of Formula 1e may be prepared according to Scheme 1. For example, heteroaryl amine 1b can be formed from chloride 1a via a nucleophilic aromatic substitution reaction. Substitution of the lactam can proceed under basic conditions to give diene 1c, which can undergo a cross metathesis reaction—such as Grubbs cross metathesis reaction—to form macrocycle 1d. Optionally, 1d may be subjected to one or more subsequent reactions, such as a hydrogenation reaction, to provide a compound of Formula 1e.

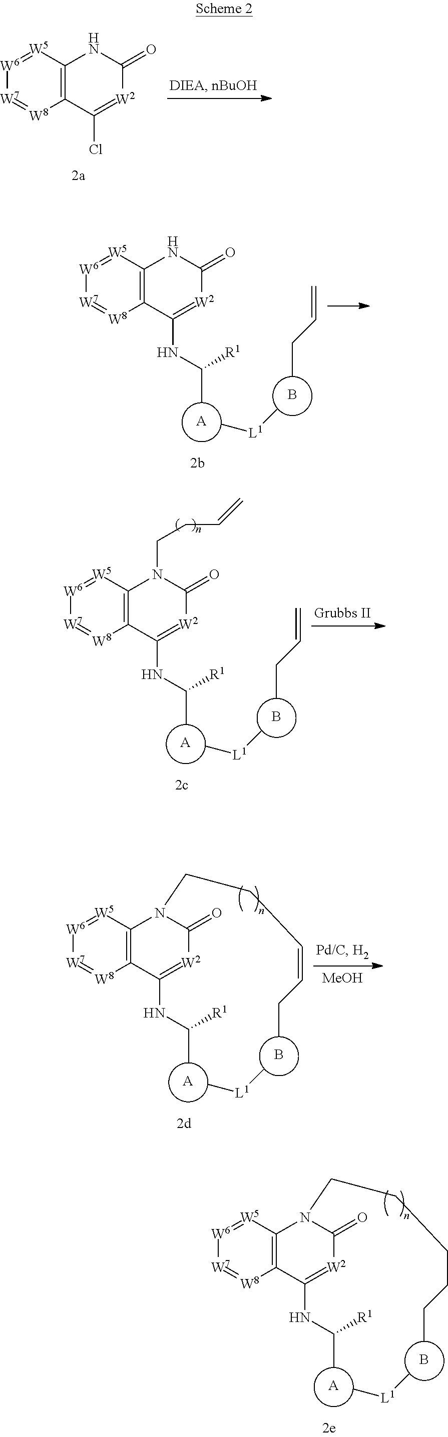

Similarly, in some embodiments, a compound of Formula 2e may be prepared according to Scheme 2. For example, heteroaryl amine 2b can be formed from chloride 2a via a nucleophilic aromatic substitution reaction. Substitution of the lactam can proceed under basic conditions to give diene 2c, which can undergo a cross metathesis reaction—such as Grubbs cross metathesis reaction—to form macrocycle 2d. Optionally, 2d may be subjected to one or more subsequent reactions, such as a hydrogenation reaction, to provide a compound of Formula 2e.

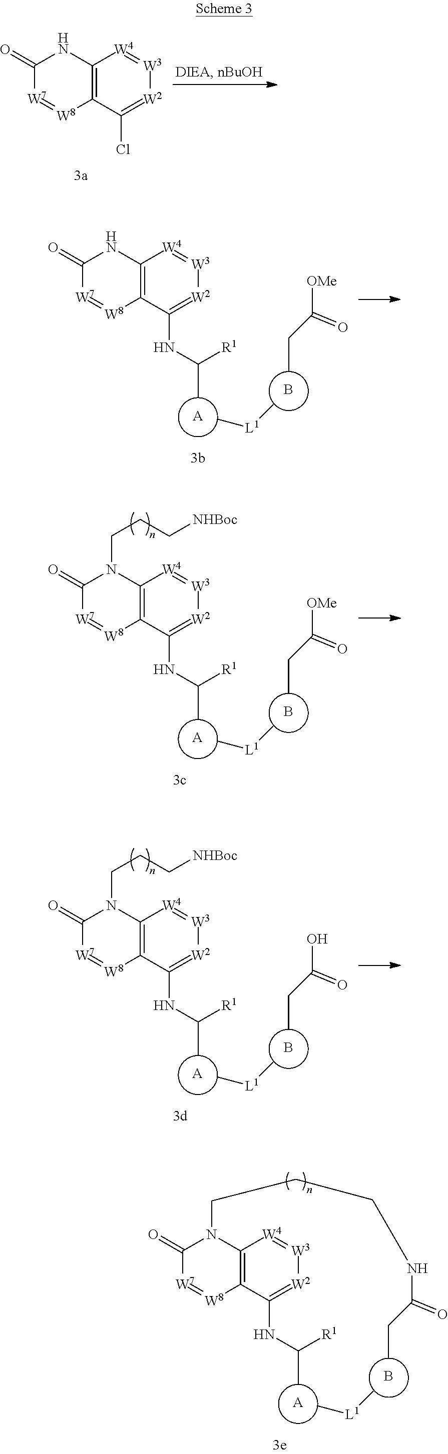

In some embodiments, a compound of Formula 3e may be prepared according to Scheme 3. For example, heteroaryl amine 3b can be formed from chloride 3a via a substitution reaction. Substitution of the lactam can give protected amine 3c. Hydrolysis of the ester can form carboxylic acid 3d, which can undergo deprotection and peptide coupling reactions to afford macrocycle of Formula 3e.

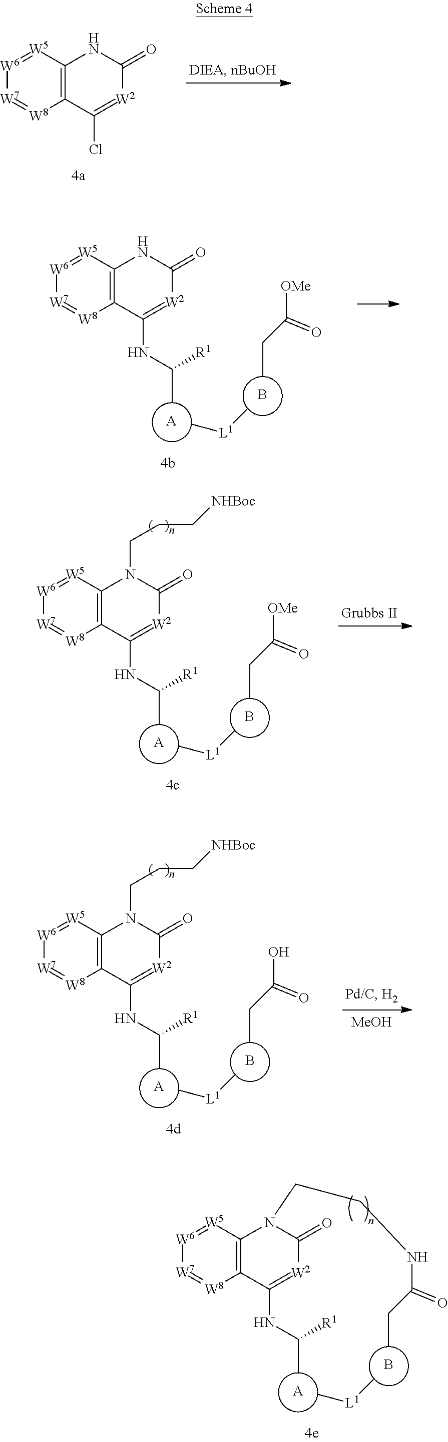

Similarly, in some embodiments, a compound of Formula 4e may be prepared according to Scheme 4. For example, heteroaryl amine 4b can be formed from chloride 4a via a substitution reaction. Substitution of the lactam can give protected amine 4c. Hydrolysis of the ester can form carboxylic acid 4d, which can undergo deprotection and peptide coupling reactions to afford a macrocycle of Formula 4e.

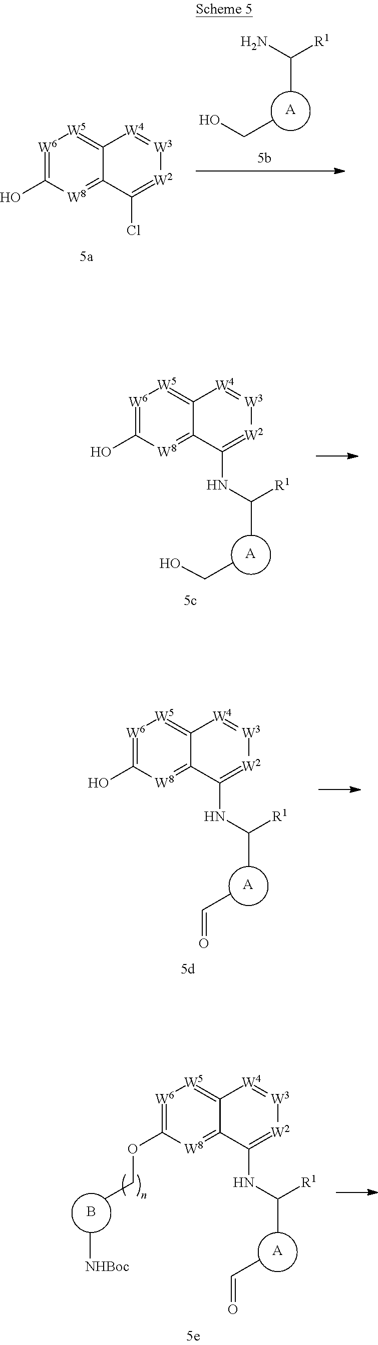

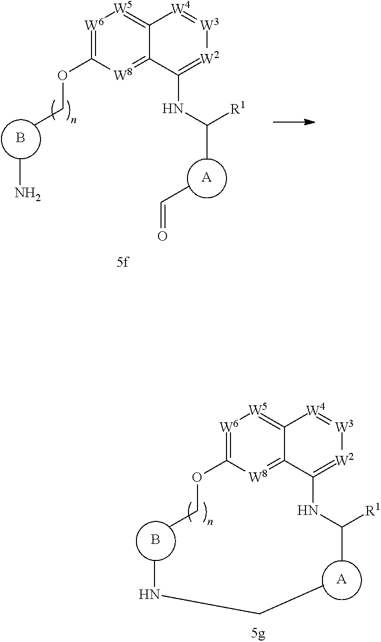

In some embodiments, a compound of Formula 5g may be prepared according to Scheme 5. For example, heteroaryl amine 3c can be formed by coupling chloride 5a with amine 5b. Oxidation of the alcohol can give aldehyde 5d, which can be followed with substitution of the phenol to give 5e. Removal of the amine protecting group can afford 5f, which can undergo a reductive amination to form a macrocycle of Formula 5g.

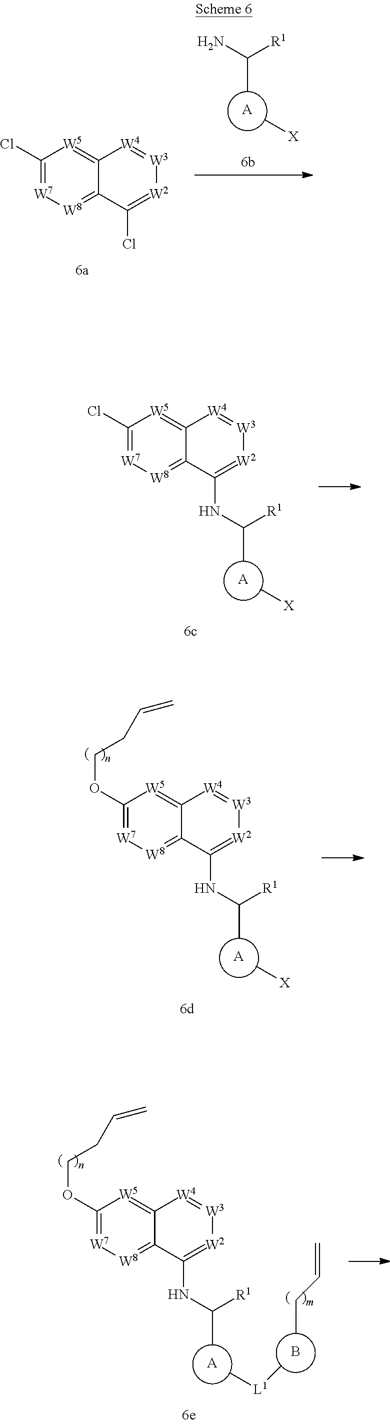

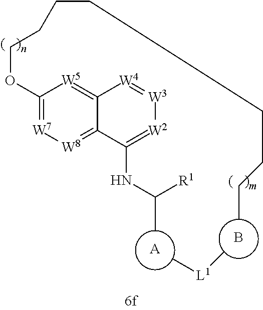

In some embodiments, a compound of Formula 6f may be prepared according to Scheme 6. For example, heteroaryl amine 6c can be formed by coupling chloride 1a with amine 6b. Substitution of the phenol to olefin 6d can be followed by installation of a second olefin to give diene 6e. A cross metathesis reaction—such as Grubbs cross metathesis reaction—can be followed by hydrogenation of the resulting double bond to provide a macrocycle of Formula 6f.

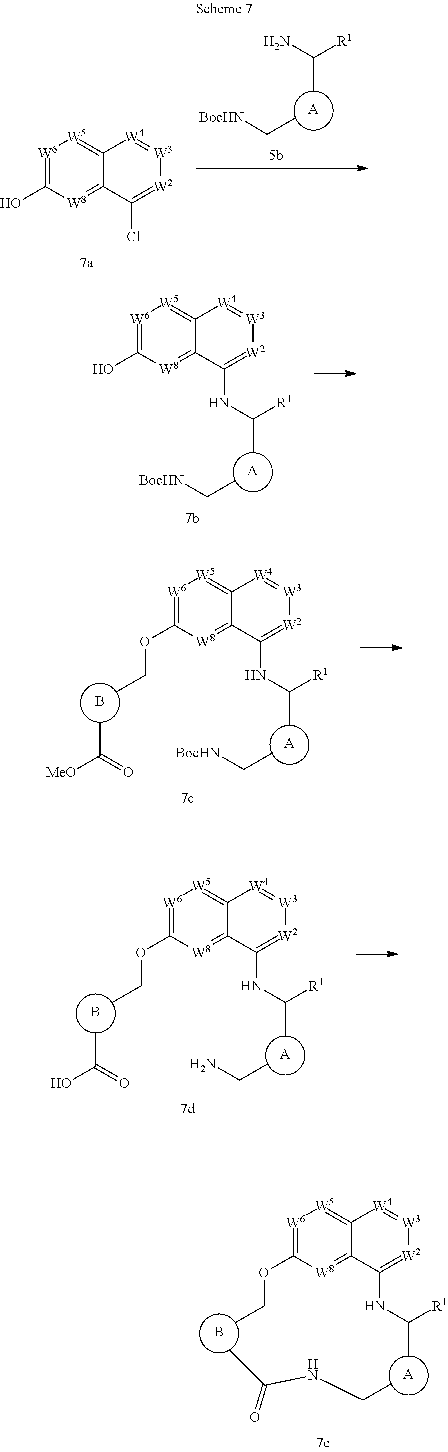

In some embodiments, a compound of Formula 7e may be prepared according to Scheme 7. For example, heteroaryl amine 7b can be formed by coupling chloride 7a with amine 5b. Substitution of the phenol can give 7c. Ester hydrolysis and deprotection of the amine can give 7d, which can be cyclized to form a macrocycle of Formula 7e via a peptide coupling reaction.

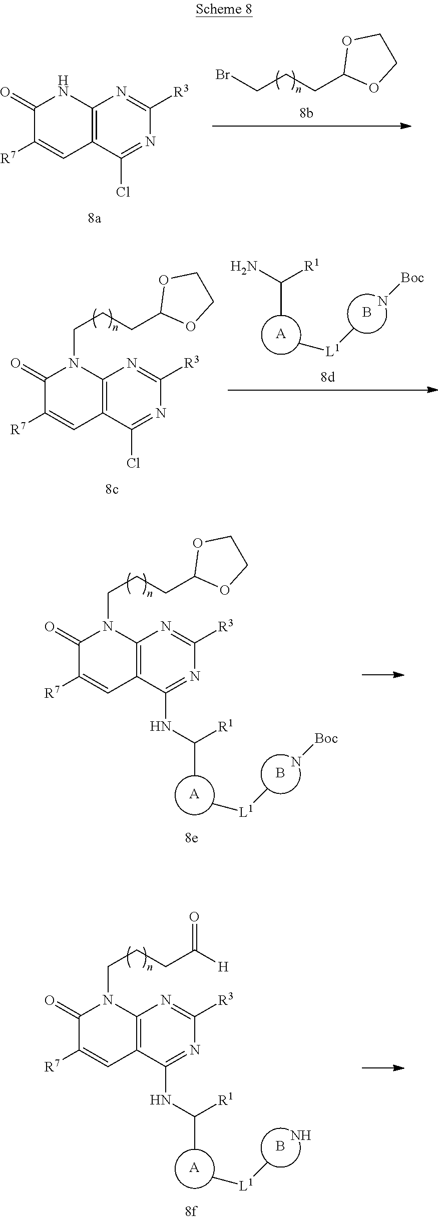

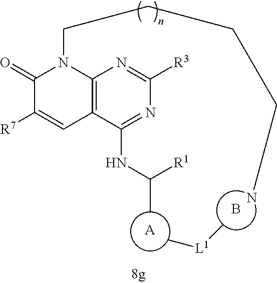

In some embodiments, a compound of Formula 8g may be prepared according to Scheme 8. For example, substitution of lactam 8a with a suitable bromo dioxolane (8b) can give acetal 8c. Nucleophilic aromatic substitution with amine 8d can provide heteroaryl amine 8e, which can be treated with a suitable acid, such as HCl, to remove the Boc protecting group and reveal the aldehyde. Finally, cyclization of 8f can proceed via reductive amination conditions to give a macrocycle of Formula 8g.

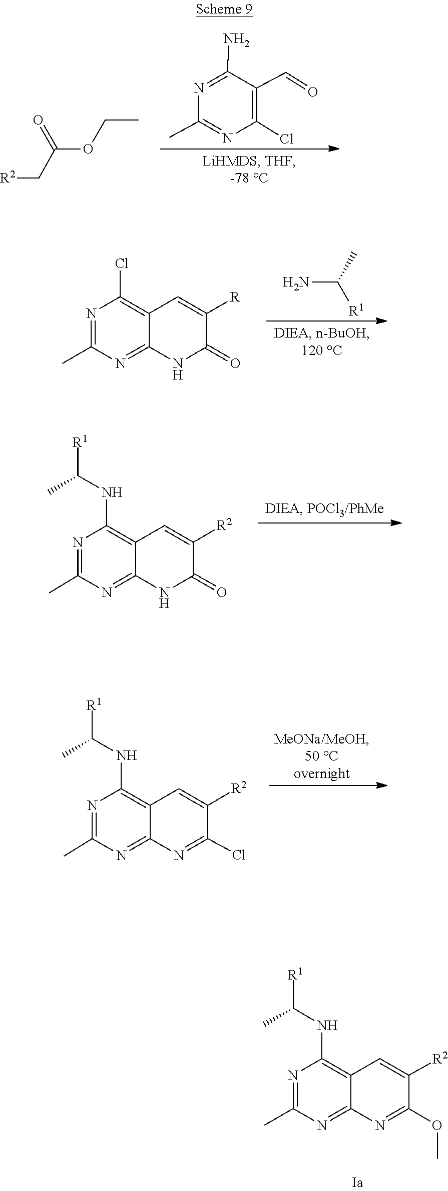

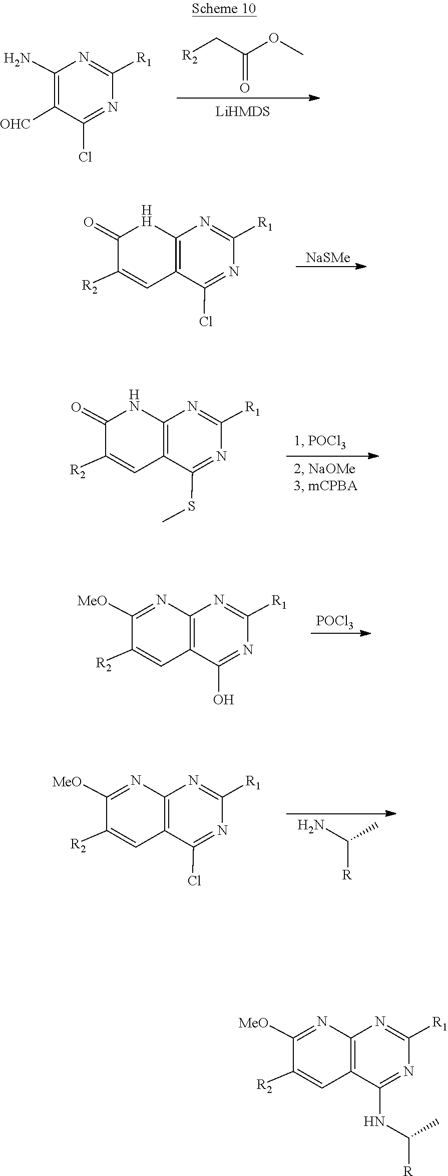

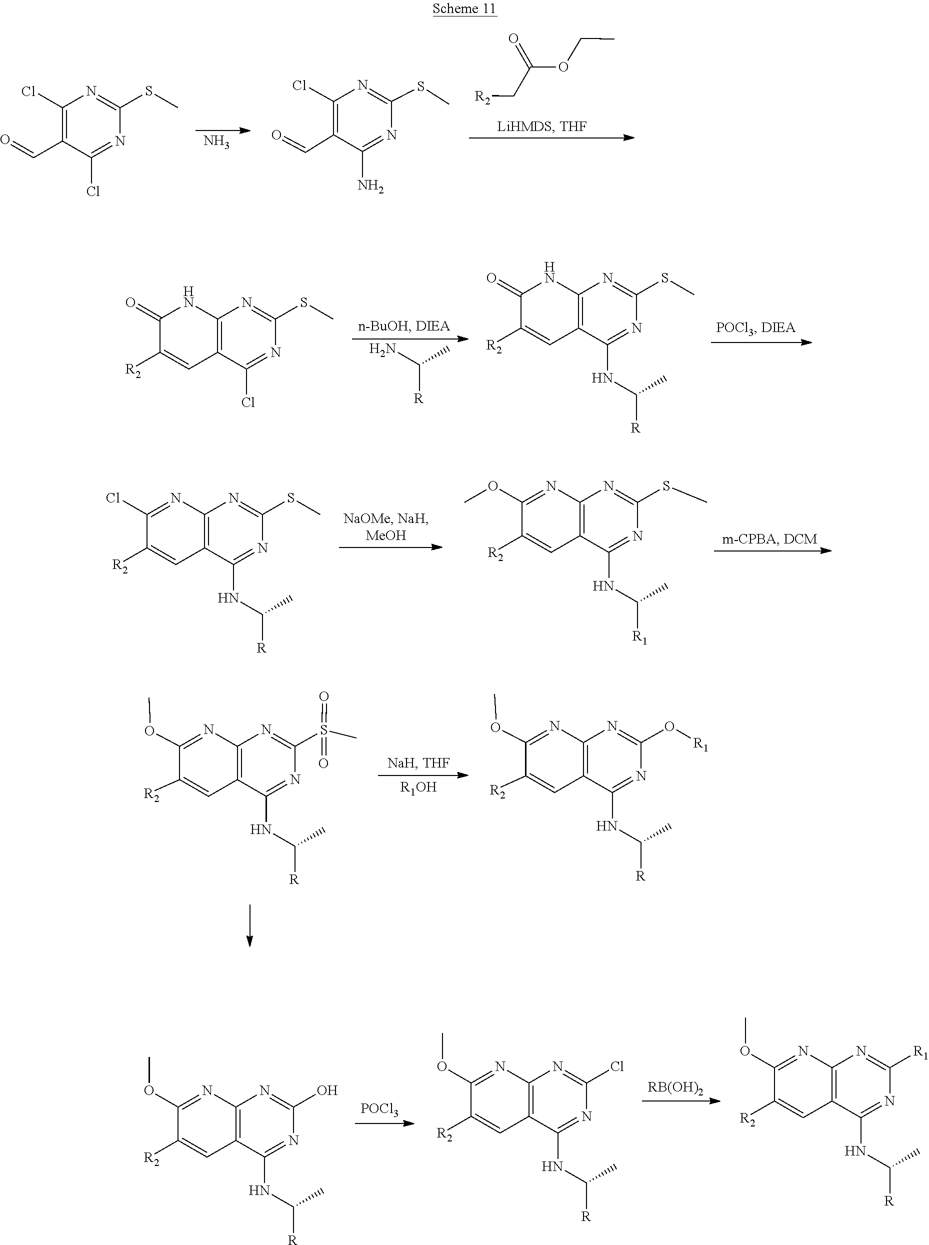

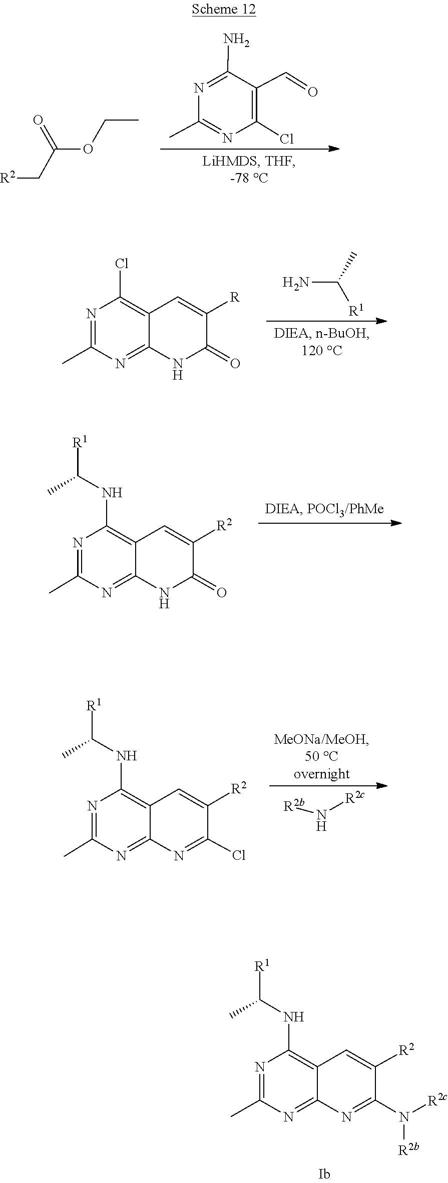

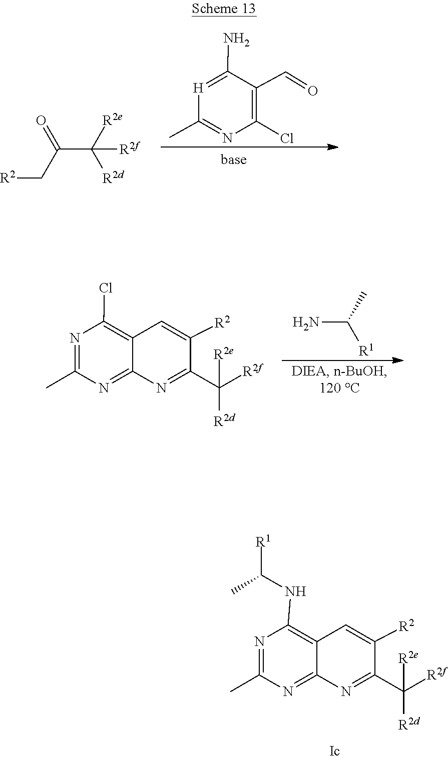

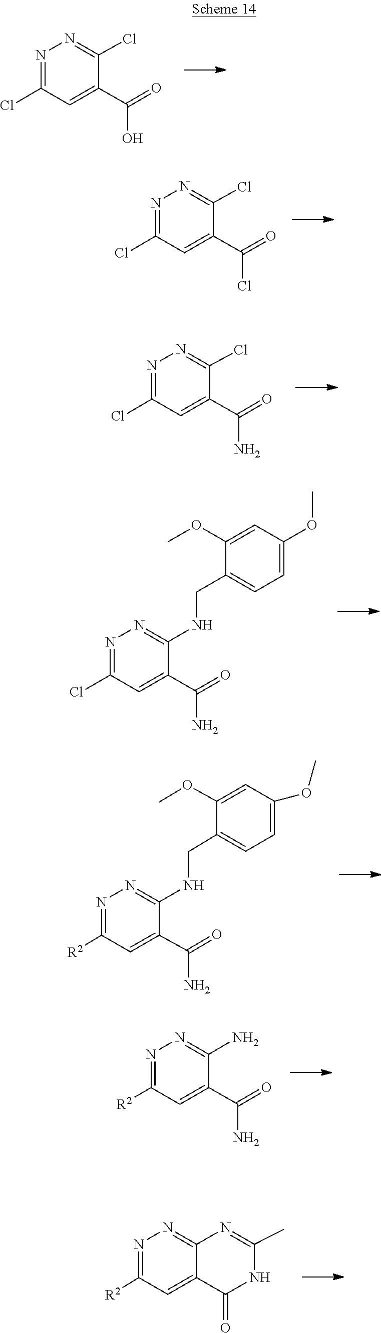

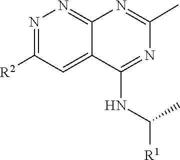

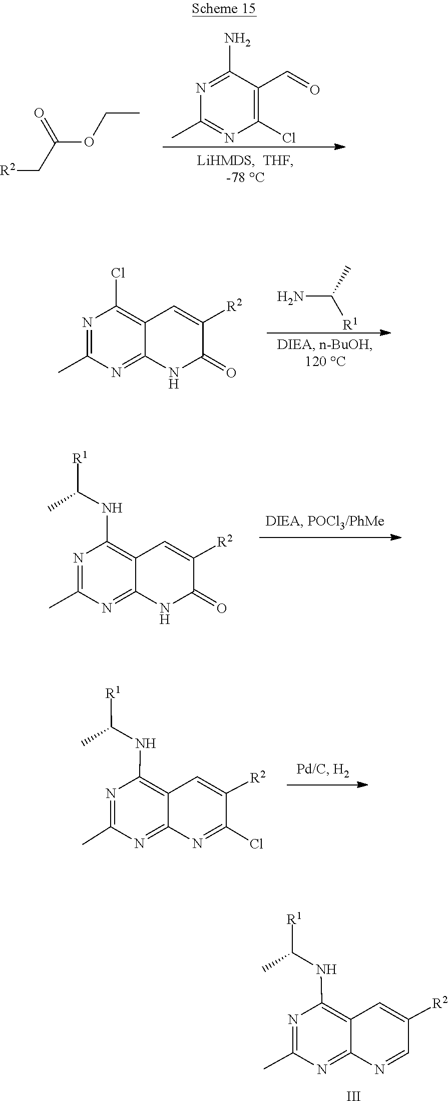

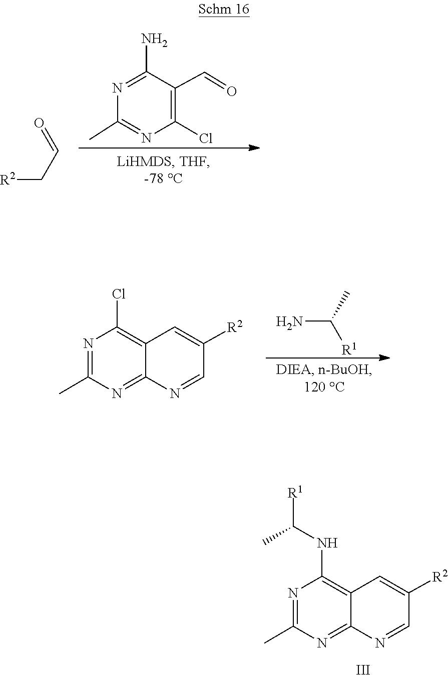

Synthetic procedures for certain compounds may be found in PCT/US2022/018584, U.S. Pat. No. 11,648,254, PCT/US2023/065963, and U.S. application Ser. No. 18/328,109, each of which is incorporated by reference in its entirety, including any compounds, formulas, recitations of compound variables, and synthetic methods disclosed therein. In some embodiments, a SOS1 inhibitor of the present disclosure is a compound described in U.S. Pat. No. 11,648,254, which is incorporated herein by reference in its entirety. In some embodiments, a SOS1 inhibitor of the present disclosure is a compound described in U.S. Pat. No. 11,912,708, which is incorporated herein by reference in its entirety.

In some embodiments, a compound of the present disclosure, for example, a compound of a formula given in Table 1 or Table 2, was synthesized according to one of the general routes outlined in Schemes 1-16 or by methods generally known in the art. In some embodiments, exemplary compounds may include, but are not limited to, a compound selected from Table 1, Table 2, or a salt or solvate thereof.

TABLE 1

| No. | Structure | Chemical Name | [M + H]⁺ |
|---|---|---|---|
| A1 | 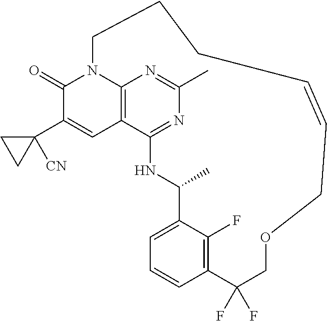 [SMILES: Cc1nc2c3cc(C4(C#N)CC4)c(=O)n(c3n1)CCC/C=C\COCC(F)(F)c1cccc(c1F)[C@@H](C)N2] | 1-((3R,Z)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclotridecaphan-9- en-1⁶-yl)cyclopropane-1-carbonitrile | 524.4 |
| A2 | 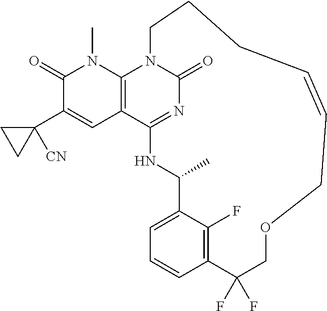 [SMILES: C[C@H]1Nc2nc(=O)n(c3c2cc(C2(C#N)CC2)c(=O)n3C)CCC/C=C\COCC(F)(F)c2cccc1c2F] | 1-((1³E,3R,9Z)-4²,5,5-trifluoro-1⁸,3-dimethyl- 1²,1⁷-dioxo-1¹,1²,1⁷,1⁸-tetrahydro-7-oxa-2-aza- 1(4,1)-pyrido[2,3-d]pyrimidina-4(1,3)- benzenacyclotridecaphan-9-en-1⁶-yl)cyclopropane- 1-carbonitrile | 540.4 |
| A3 | 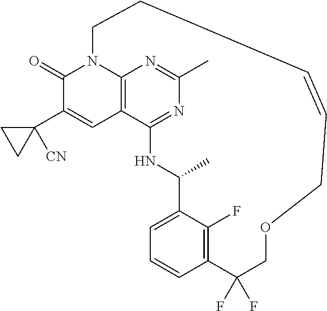 [SMILES: Cc1nc2c3cc(C4(C#N)CC4)c(=O)n(c3n1)CC/C=C\COCC(F)(F)c1cccc(c1F)[C@@H](C)N2] | 1-((3R,Z)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclododecaphan-9- en-1⁶-yl)cyclopropane-1-carbonitrile | 510.4 |
| A4 | 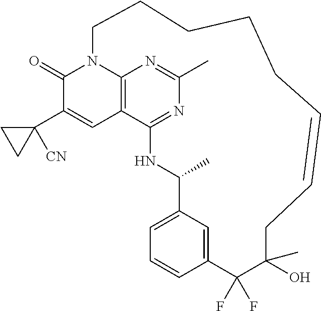 [SMILES: Cc1nc2c3cc(C4(C#N)CC4)c(=O)n(c3n1)CCCCC/C=C\CC(C)(O)C(F)(F)c1cccc(c1)[C@@H](C)N2] | 1-((3R,Z)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclotetradecaphan-8- en-1⁶-yl)cyclopropane-1-carbonitrile | 548.3 |
| A5 | 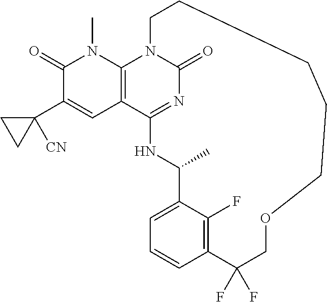 [SMILES: C[C@H]1Nc2nc(=O)n(c3c2cc(C2(C#N)CC2)c(=O)n3C)CCCCCOCC(F)(F)c2cccc1c2F] | 1-((3R,E)-4²,5,5-trifluoro-1⁸,3-dimethyl-1²,1⁷- dioxo-1¹,1²,1⁷,1⁸-tetrahydro-7-oxa-2-aza-1(4,1)- pyrido[2,3-d]pyrimidina-4(1,3)- benzenacyclododecaphane-1⁶-yl)cyclopropane-1- carbonitrile | 528.4 |
| A6 | 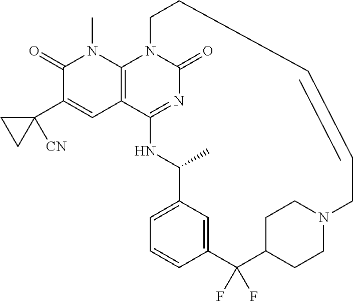 [SMILES: C[C@H]1Nc2nc(=O)n(c3c2cc(C2(C#N)CC2)c(=O)n3C)CC/C=C\CN2CCC(CC2)C(F)(F)c2cccc1c2] | 1-((6³E,4R,9Z)-2,2-difluoro-6⁸,4-dimethyl-6²,6⁷- dioxo-6¹,6²,6⁷,6⁸-tetrahydro-5-aza-6(4,1)- pyrido[2,3-d]pyrimidina-1(4,1)-piperidina-3(1,3)- benzenacycloundecaphan-9-en-6⁶- yl)cyclopropane-1-carbonitrile | 561.4 |
| A7 | 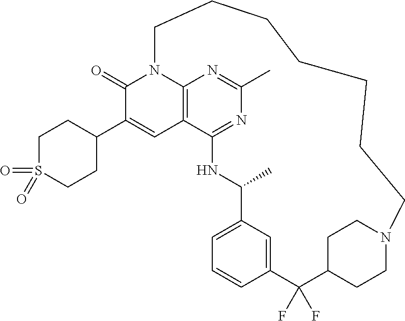 [SMILES: Cc1nc2c3cc(C4CCS(=O)(=O)CC4)c(=O)n(c3n1)CCCCCCCN1CCC(CC1)C(F)(F)c1cccc(c1)[C@@H](C)N2] | (4R)-6⁶-(1,1-dioxidotetrahydro-2H-thiopyran-4- yl)-2,2-difluoro-6²,4-dimethyl-6⁷,6⁸-dihydro-5-aza- 6(4,8)-pyrido[2,3-d]pyrimidina-1(4,1)-piperidina- 3(1,3)-benzenacyclotridecaphan-6⁷-one | 644.5 |
| A8 | 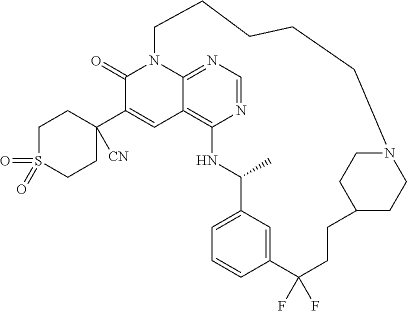 [SMILES: C[C@H]1Nc2ncnc3c2cc(C2(C#N)CCS(=O)(=O)CC2)c(=O)n3CCCCCN2CCC(CC2)CCC(F)(F)c2cccc1c2] | 4-((3R)-5,5-difluoro-3-methyl-1⁷-oxo-1⁷,1⁸- dihydro-2-aza-1(4,8)-pyrido[2,3-d]pyrimidina- 8(4,1)-piperidina-4(1,3)- benzenacyclotridecaphane-1⁶-yl)tetrahydro-2H- thiopyran-4-carbonitrile 1,1-dioxide | 653.3 |
| A9 | 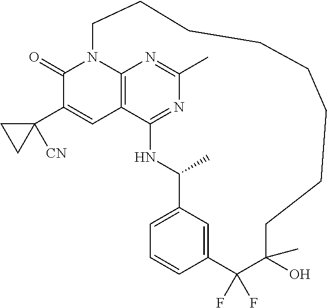 [SMILES: Cc1nc2c3cc(C4(C#N)CC4)c(=O)n(c3n1)CCCCCCCCC(C)(O)C(F)(F)c1cccc(c1)[C@@H](C)N2] | 1-((3R)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclotetradecaphane- 1⁶-yl)cyclopropane-1-carbonitrile | 550.4 |
| A10 | 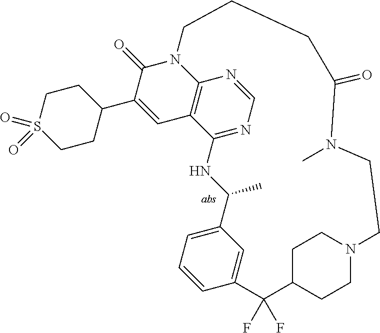 [SMILES: C[C@H]1Nc2ncnc3c2cc(C2CCS(=O)(=O)CC2)c(=O)n3CCCC(=O)N(C)CCN2CCC(CC2)C(F)(F)c2cccc1c2] | (4R)-6⁶-(1,1-dioxidotetrahydro-2H-thiopyran-4- yl)-2,2-difluoro-4,11-dimethyl-6⁷,6⁸-dihydro-5,11- diaza-6(4,8)-pyrido[2,3-d]pyrimidina-1(4,1)- piperidina-3(1,3)-benzenacyclotridecaphane-6⁷,10- dione | 657.4 |
| A11 | 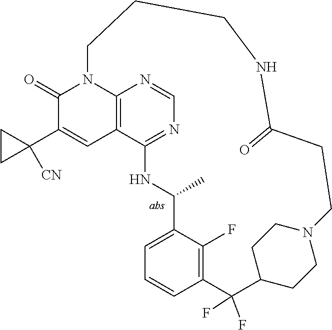 [SMILES: C[C@H]1Nc2ncnc3c2cc(C2(C#N)CC2)c(=O)n3CCCNC(=O)CCN2CCC(CC2)C(F)(F)c2cccc1c2F] | 1-((4R)-3²,2,2-trifluoro-4-methyl-6⁷,11-dioxo- 6⁷,6⁸-dihydro-5,10-diaza-6(4,8)-pyrido[2,3- d]pyrimidina-1(4,1)-piperidina-3(1,3)- benzenacyclotridecaphane-6⁶-yl)cyclopropane-1- carbonitrile | 594.5 |
| A12 | 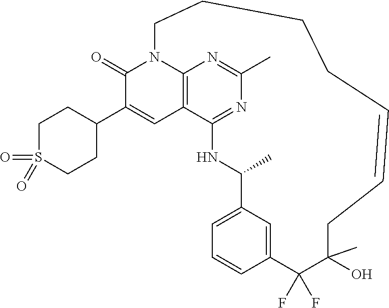 [SMILES: Cc1nc2c3cc(C4CCS(=O)(=O)CC4)c(=O)n(c3n1)CCCC/C=C\CC(C)(O)C(F)(F)c1cccc(c1)[C@@H](C)N2] | (3R,Z)-1⁶-(1,1-dioxidotetrahydro-2H-thiopyran-4- yl)-5,5-difluoro-6-hydroxy-1²,3,6-trimethyl-1⁷,1⁸- dihydro-2-aza-1(4,8)-pyrido[2,3-d]pyrimidina- 4(1,3)-benzenacyclotridecaphan-8-en-1⁷-one | 601.4 |
| A13 | 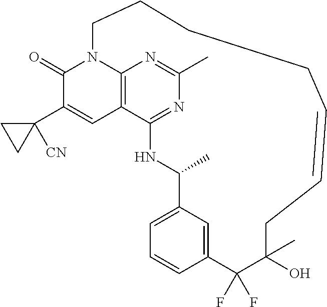 [SMILES: Cc1nc2c3cc(C4(C#N)CC4)c(=O)n(c3n1)CCCC/C=C\CC(C)(O)C(F)(F)c1cccc(c1)[C@@H](C)N2] | 1-((3R,Z)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclotridecaphan-8- en-1⁶-yl)cyclopropane-1-carbonitrile | 534.4 |
| A14 | 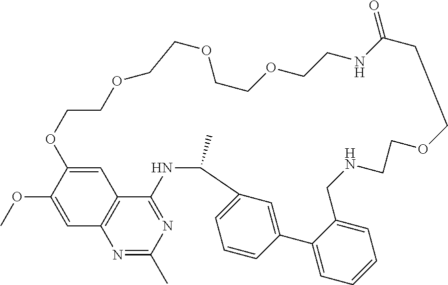 [SMILES: COc1cc2nc(C)nc3c2cc1OCCOCCOCCOCCNC(=O)CCOCCNCc1ccccc1-c1cccc(c1)[C@@H](C)N3] | (R)-5⁷-methoxy-5²,3-dimethyl-6,9,12,15,22- pentaoxa-4,18,25-triaza-5(4,6)-quinazolina- 1(1,2),2(1,3)-dibenzenacyclohexacosaphan-19-one | 688.4 |
| A15 | 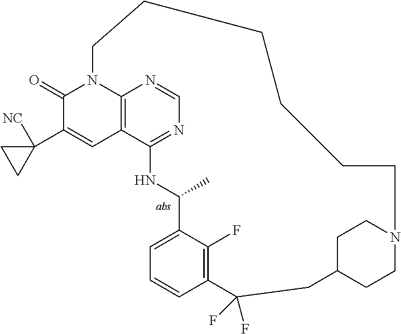 [SMILES: C[C@H]1Nc2ncnc3c2cc(C2(C#N)CC2)c(=O)n3CCCCCCN2CCC(CC2)CC(F)(F)c2cccc1c2F] | 1-((3R)-4²,5,5-trifluoro-3-methyl-1⁷-oxo-1⁷,1⁸- dihydro-2-aza-1(4,8)-pyrido[2,3-d]pyrimidina- 7(4,1)-piperidina-4(1,3)- benzenacyclotridecaphane-1⁶-yl)cyclopropane-1- carbonitrile | 579.4 |
| A16 | 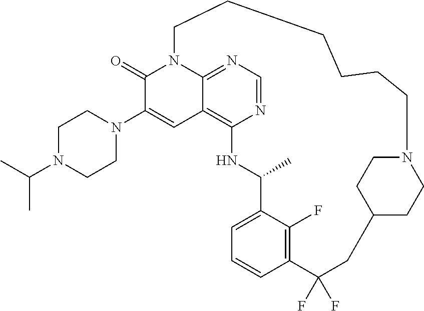 [SMILES: CC(C)N1CCN(c2cc3c4ncnc3n(c2=O)CCCCCCN2CCC(CC2)CC(F)(F)c2cccc(c2F)[C@@H](C)N4)CC1] | (3R)-4²,5,5-trifluoro-1⁶-(4-isopropylpiperazin-1- yl)-3-methyl-1⁷,1⁸-dihydro-2-aza-1(4,8)- pyrido[2,3-d]pyrimidina-7(4,1)-piperidina-4(1,3)- benzenacyclotridecaphan-1⁷-one | 640.8 |
| A17 | 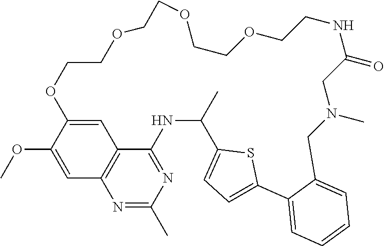 [SMILES: COc1cc2nc(C)nc3c2cc1OCCOCCOCCOCCNC(=O)CN(C)Cc1ccccc1C1=CC=C(S1)C(C)N3] | 5⁷-methoxy-5²,3,21-trimethyl-6,9,12,15-tetraoxa- 4,1⁸,21-triaza-5(4,6)-quinazolina-2(2,5)- thiophena-1(1,2)-benzenacyclodocosaphan-19-one | 650.3 |
| A18 | 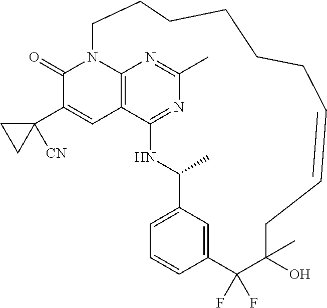 [SMILES: Cc1nc2c3cc(C4(C#N)CC4)c(=O)n(c3n1)CCCCCC/C=C\CC(C)(O)C(F)(F)c1cccc(c1)[C@@H](C)N2] | 1-((3R,Z)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclopentadecaphan- 8-en-1⁶-yl)cyclopropane-1-carbonitrile | 562.4 |
| A19 | 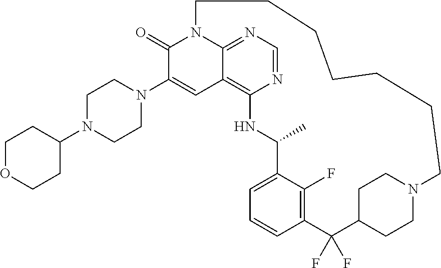 [SMILES: C[C@H]1Nc2ncnc3c2cc(N2CCN(C4CCOCC4)CC2)c(=O)n3CCCCCCCN2CCC(CC2)C(F)(F)c2cccc1c2F] | (4R)-3²,2,2-trifluoro-4-methyl-6⁶-(4-(tetrahydro- 2H-pyran-4-yl)piperazin-1-yl)-6⁷,6⁸-dihydro-5- aza-6(4,8)-pyrido[2,3-d]pyrimidina-1(4,1)- piperidina-3(1,3)-benzenacyclotridecaphan-6⁷-one | 682.5 |
| A20 | 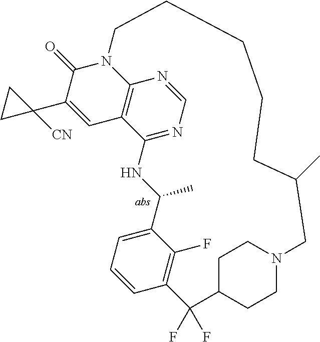 [SMILES: CC1CCCCCn2c(=O)c(C3(C#N)CC3)cc3c(ncnc32)N[C@H](C)c2cccc(c2F)C(F)(F)C2CCN(CC2)C1] | 1-((4R)-3²,2,2-trifluoro-4,12-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)cyclopropane-1- carbonitrile | 593.4 |
| A21 | 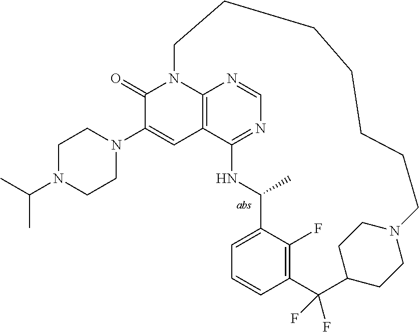 [SMILES: CC(C)N1CCN(c2cc3c4ncnc3n(c2=O)CCCCCCCN2CCC(CC2)C(F)(F)c2cccc(c2F)[C@@H](C)N4)CC1] | (4R)-3²,2,2-trifluoro-6⁶-(4-isopropylpiperazin-1- yl)-4-methyl-6⁷,6⁸-dihydro-5-aza-6(4,8)- pyrido[2,3-d]pyrimidina-1(4,1)-piperidina-3(1,3)- benzenacyclotridecaphan-6⁷-one | 640.3 |
| A22 | 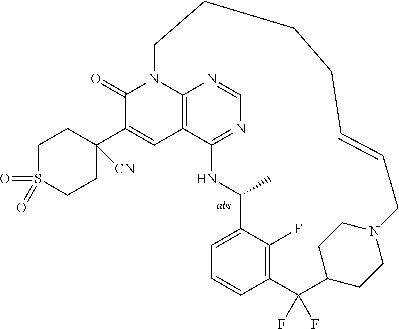 [SMILES: C[C@H]1Nc2ncnc3c2cc(C2(C#N)CCS(=O)(=O)CC2)c(=O)n3CCCC/C=C/CN2CCC(CC2)C(F)(F)c2cccc1c2F] | 4-((4R,E)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)-benzenacyclotridecaphan- 11-en-6⁶-yl)tetrahydro-2H-thiopyran-4-carbonitrile 1,1-dioxide | 651.3 |
| A23 | 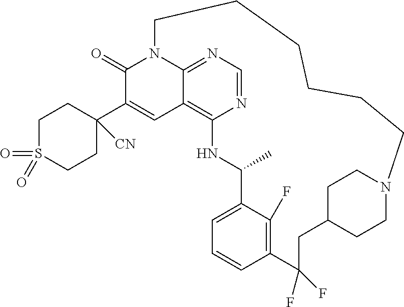 [SMILES: C[C@H]1Nc2ncnc3c2cc(C2(C#N)CCS(=O)(=O)CC2)c(=O)n3CCCCCCN2CCC(CC2)CC(F)(F)c2cccc1c2F] | 4-((3R)-4²,5,5-trifluoro-3-methyl-1⁷-oxo-1⁷,1⁸- dihydro-2-aza-1(4,8)-pyrido[2,3-d]pyrimidina- 7(4,1)-piperidina-4(1,3)- benzenacyclotridecaphane-1⁶-yl)tetrahydro-2H- thiopyran-4-carbonitrile 1,1-dioxide | 671.3 |
| A24 | 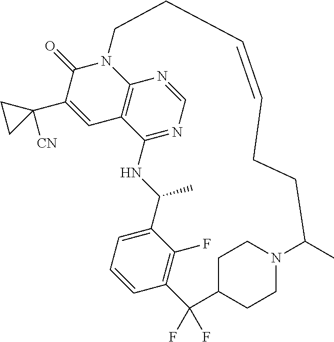 [SMILES: CC1CC/C=C\CCn2c(=O)c(C3(C#N)CC3)cc3c(ncnc32)N[C@H](C)c2cccc(c2F)C(F)(F)C2CCN1CC2] | 1-((4R,Z)-3²,2,2-trifluoro-4,13-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphan-9-en-6⁶-yl)cyclopropane- 1-carbonitrile | 591.5 |
| A25 | 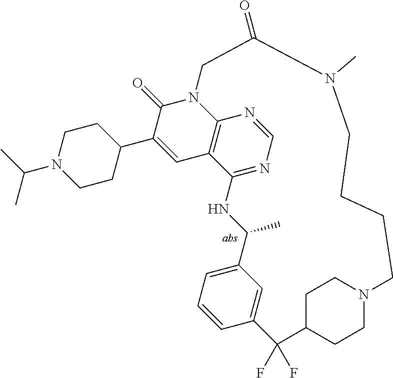 [SMILES: CC(C)N1CCC(c2cc3c4ncnc3n(c2=O)CC(=O)N(C)CCCCN2CCC(CC2)C(F)(F)c2cccc(c2)[C@@H](C)N4)CC1] | (4R)-2,2-difluoro-6⁶-(1-isopropylpiperidin-4-yl)- 4,9-dimethyl-6⁷,6⁸-dihydro-5,9-diaza-6(4,8)- pyrido[2,3-d]pyrimidina-1(4,1)-piperidina-3(1,3)- benzenacyclotridecaphane-6⁷,8-dione | 650.3 |
| A26 | 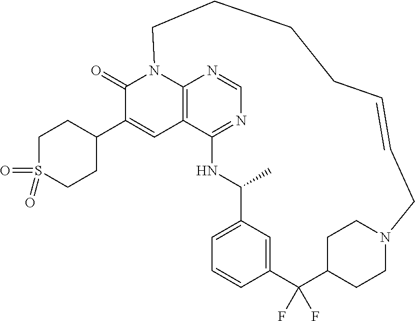 [SMILES: C[C@H]1Nc2ncnc3c2cc(C2CCS(=O)(=O)CC2)c(=O)n3CCCC/C=C/CN2CCC(CC2)C(F)(F)c2cccc1c2] | (4R,E)-6⁶-(1,1-dioxidotetrahydro-2H-thiopyran-4- yl)-2,2-difluoro-4-methyl-6⁷,6⁸-dihydro-5-aza- 6(4,8)-pyrido[2,3-d]pyrimidina-1(4,1)-piperidina- 3(1,3)-benzenacyclotridecaphan-11-en-6⁷-one | 626.4 |
| A27 | 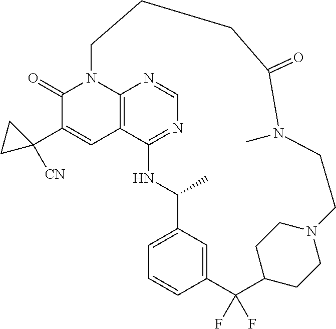 [SMILES: C[C@H]1Nc2ncnc3c2cc(C2(C#N)CC2)c(=O)n3CCCC(=O)N(C)CCN2CCC(CC2)C(F)(F)c2cccc1c2] | 1-((4R)-2,2-difluoro-4,11-dimethyl-6⁷,10-dioxo- 6⁷,6⁸-dihydro-5,11-diaza-6(4,8)-pyrido[2,3- d]pyrimidina-1(4,1)-piperidina-3(1,3)- benzenacyclotridecaphane-6⁶-yl)cyclopropane-1- carbonitrile | 590.3 |
| A28 | 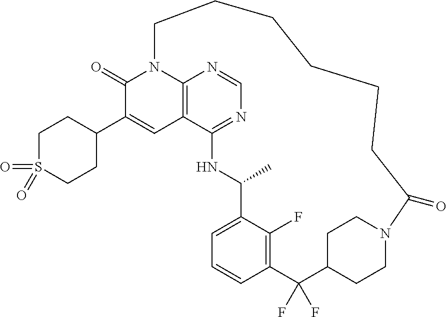 [SMILES: C[C@H]1Nc2ncnc3c2cc(C2CCS(=O)(=O)CC2)c(=O)n3CCCCCCC(=O)N2CCC(CC2)C(F)(F)c2cccc1c2F] | (4R)-6⁶-(1,1-dioxidotetrahydro-2H-thiopyran-4- yl)-3²,2,2-trifluoro-4-methyl-6⁷,6⁸-dihydro-5-aza- 6(4,8)-pyrido[2,3-d]pyrimidina-1(4,1)-piperidina- 3(1,3)-benzenacyclotridecaphane-6⁷,13-dione | 660.2 |
| A29 | 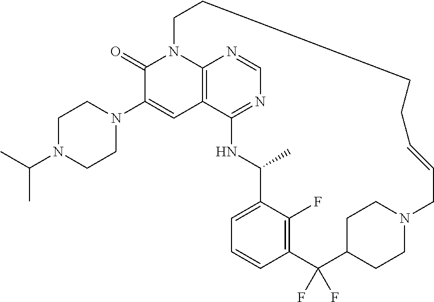 [SMILES: CC(C)N1CCN(c2cc3c4ncnc3n(c2=O)CCCC/C=C/CN2CCC(CC2)C(F)(F)c2cccc(c2F)[C@@H](C)N4)CC1] | (4R,E)-2,2-difluoro-6⁶-(4-isopropylpiperazin-1-yl)- 4-methyl-6⁷,6⁸-dihydro-5-aza-6(4,8)-pyrido[2,3- d]pyrimidina-1(4,1)-piperidina-3(1,3)- benzenacyclotridecaphan-11-en-6⁷-one | 620.3 |
| A30 | 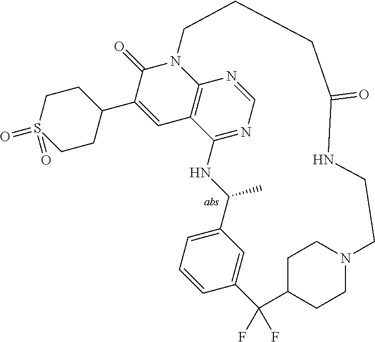 [SMILES: C[C@H]1Nc2ncnc3c2cc(C2CCS(=O)(=O)CC2)c(=O)n3CCCC(=O)NCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5,11- diaza-6(4,8)-pyrido[2,3-d]pyrimidina-1(4,1)- piperidina-3(1,3)-benzenacyclotridecaphane-6⁷,10- dione | 643.4 |
| A31 | 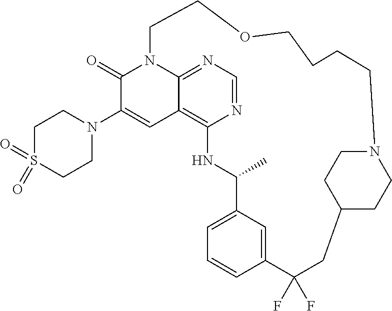 [SMILES: C[C@H]1Nc2ncnc3c2cc(N2CCS(=O)(=O)CC2)c(=O)n3CCOCCCCN2CCC(CC2)CC(F)(F)c2cccc1c2] | (3R)-1⁶-(1,1-dioxidotetrahydro-2H-thiopyran-4- yl)-5,5-difluoro-3-methyl-1⁷,1⁸-dihydro-12-oxa-2- aza-1(4,8)-pyrido[2,3-d]pyrimidina-7(4,1)- piperidina-4(1,3)-benzenacyclotetradecaphan-1⁷- one | 664.5 |
| A32 | 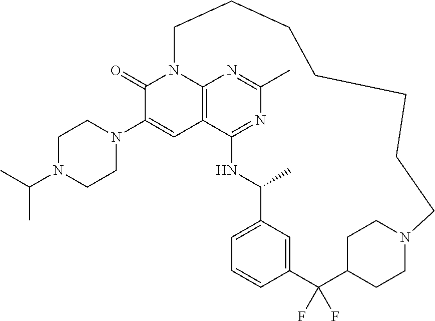 [SMILES: Cc1nc2c3cc(N4CCN(C(C)C)CC4)c(=O)n(c3n1)CCCCCCCN1CCC(CC1)C(F)(F)c1cccc(c1)[C@@H](C)N2] | (4R)-2,2-difluoro-6⁶-(1-isopropylpiperidin-4-yl)- 6²,4-dimethyl-6⁷,6⁸-dihydro-5-aza-6(4,8)- pyrido[2,3-d]pyrimidina-1(4,1)-piperidina-3(1,3)- benzenacyclotridecaphan-6⁷-one | 635.6 |
| A33 | 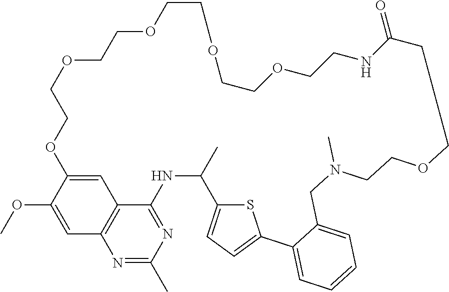 [SMILES: COc1cc2nc(C)nc3c2cc1OCCOCCOCCOCCOCCNC(=O)CCOCCN(C)Cc1ccccc1-c1ccc(s1)C(C)N3] | 5⁷-methoxy-5²,3,28-trimethyl-6,9,1²,15,1⁸,25- hexaoxa-4,21,28-triaza-5(4,6)-quinazolina-2(2,5)- thiophena-1(1,2)-benzenacyclononacosaphan-22- one | 752.4 |
| A34 | 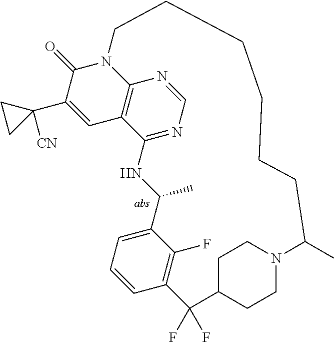 [SMILES: CC1CCCCCCn2c(=O)c(C3(C#N)CC3)cc3c(ncnc32)N[C@H](C)c2cccc(c2F)C(F)(F)C2CCN1CC2] | 1-((4R)-3²,2,2-trifluoro-4,13-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)cyclopropane-1- carbonitrile | 593.4 |
| A35 | 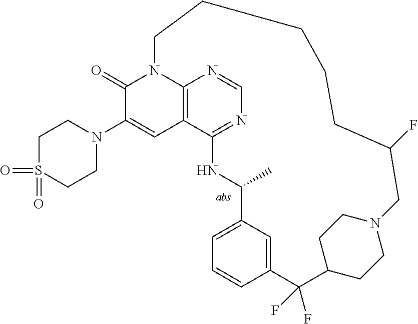 [SMILES: C[C@H]1Nc2ncnc3c2cc(N2CCS(=O)(=O)CC2)c(=O)n3CCCCCC(F)CN2CCC(CC2)C(F)(F)c2cccc1c2] | (4R)-6⁶-(1,1-dioxidotetrahydro-2H-thiopyran-4- yl)-2,2,12-trifluoro-4-methyl-6⁷,6⁸-dihydro-5-aza- 6(4,8)-pyrido[2,3-d]pyrimidina-1(4,1)-piperidina- 3(1,3)-benzenacyclotridecaphan-6⁷-one | 646.4 |
| A36 | 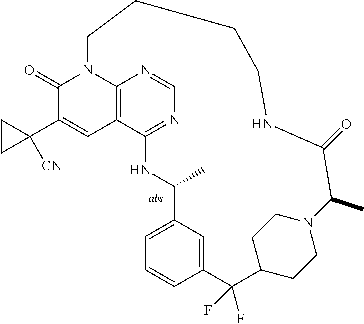 [SMILES: C[C@@H]1C(=O)NCCCCn2c(=O)c(C3(C#N)CC3)cc3c(ncnc32)N[C@H](C)c2cccc(c2)C(F)(F)C2CCN1CC2] | 1-((4R,13R)-2,2-difluoro-4,13-dimethyl-6⁷,12- dioxo-6⁷,6⁸-dihydro-5,11-diaza-6(4,8)-pyrido[2,3- d]pyrimidina-1(4,1)-piperidina-3(1,3)- benzenacyclotridecaphane-6⁶-yl)cyclopropane-1- carbonitrile | 590.3 |
| A37 | 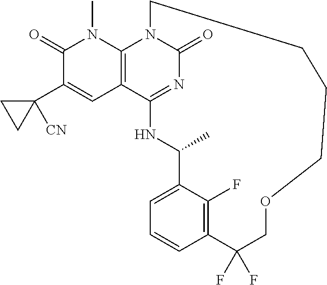 [SMILES: C[C@H]1Nc2nc(=O)n(c3c2cc(C2(C#N)CC2)c(=O)n3C)CCCCOCC(F)(F)c2cccc1c2F] | 1-((3R,E)-4²,5,5-trifluoro-1⁸,3-dimethyl-1²,1⁷- dioxo-1¹,1²,1⁷,1⁸-tetrahydro-7-oxa-2-aza-1(4,1)- pyrido[2,3-d]pyrimidina-4(1,3)- benzenacycloundecaphane-1⁶-yl)cyclopropane-1- carbonitrile | 514.3 |
| A38 | 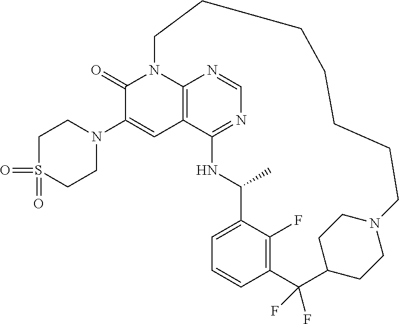 [SMILES: C[C@H]1Nc2ncnc3c2cc(N2CCS(=O)(=O)CC2)c(=O)n3CCCCCCCN2CCC(CC2)C(F)(F)c2cccc1c2F] | (4R)-6⁶-(1,1-dioxidothiomorpholino)-3²,2,2- trifluoro-4-methyl-6⁷,6⁸-dihydro-5-aza-6(4,8)- pyrido[2,3-d]pyrimidina-1(4,1)-piperidina-3(1,3)- benzenacyclotridecaphan-6⁷-one | 647.6 |
| A39 | 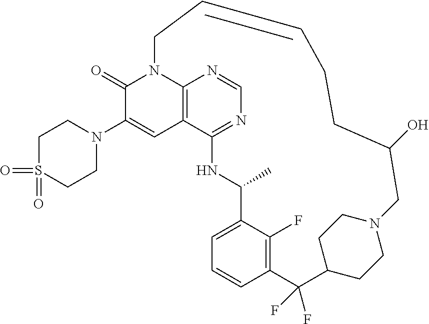 [SMILES: C[C@H]1Nc2ncnc3c2cc(N2CCS(=O)(=O)CC2)c(=O)n3C/C=C\CCC(O)CN2CCC(CC2)C(F)(F)c2cccc1c2F] | (4R,Z)-6⁶-(1,1-dioxidotetrahydro-2H-thiopyran-4- yl)-3²,2,2-trifluoro-12-hydroxy-4-methyl-6⁷,6⁸- dihydro-5-aza-6(4,8)-pyrido[2,3-d]pyrimidina- 1(4,1)-piperidina-3(1,3)-benzenacyclotridecaphan- 8-en-6⁷-one | 660.4 |
| A40 | 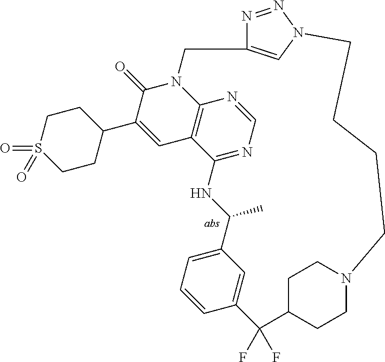 [SMILES: C[C@H]1Nc2ncnc3c2cc(C2CCS(=O)(=O)CC2)c(=O)n3Cc2cn(nn2)CCCCN2CCC(CC2)C(F)(F)c2cccc1c2] | (5R,Z)-36-(1,1-dioxidotetrahydro-2H-thiopyran-4- yl)-7,7-difluoro-5-methyl-3⁷,3⁸-dihydro-1¹H-4-aza- 3(8,4)-pyrido[2,3-d]pyrimidina-8(4,1)-piperidina- 1(4,1)-triazola-6(1,3)-benzenacyclododecaphan-37- one | 667.3 |
| A41 | 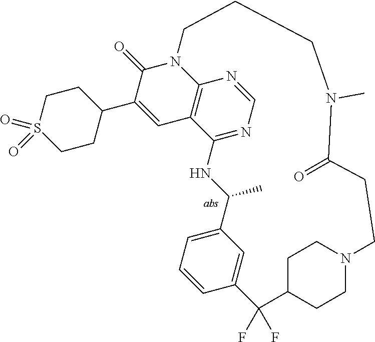 [SMILES: C[C@H]1Nc2ncnc3c2cc(C2CCS(=O)(=O)CC2)c(=O)n3CCCN(C)C(=O)CCN2CCC(CC2)C(F)(F)c2cccc1c2] | (4R)-6⁶-(1,1-dioxidotetrahydro-2H-thiopyran-4- yl)-2,2-difluoro-4,10-dimethyl-6⁷,6⁸-dihydro-5,10- diaza-6(4,8)-pyrido[2,3-d]pyrimidina-1(4,1)- piperidina-3(1,3)-benzenacyclotridecaphane-6⁷,11- dione | 657.4 |
| A42 | 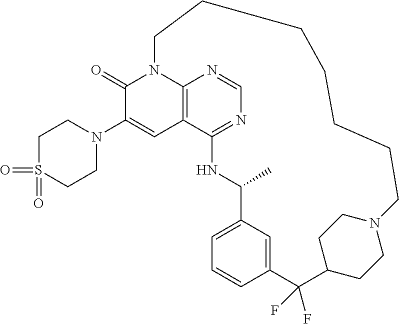 [SMILES: C[C@H]1Nc2ncnc3c2cc(N2CCS(=O)(=O)CC2)c(=O)n3CCCCCCCN2CCC(CC2)C(F)(F)c2cccc1c2] | (4R)-6⁶-(1,1-dioxidothiomorpholino)-2,2-difluoro- 4-methyl-6⁷,6⁸-dihydro-5-aza-6(4,8)-pyrido[2,3- d]pyrimidina-1(4,1)-piperidina-3(1,3)- benzenacyclotridecaphan-6⁷-one | 629.3 |
| A43 | 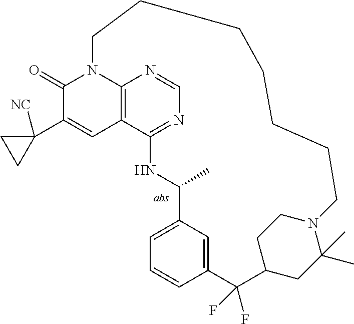 [SMILES: C[C@H]1Nc2ncnc3c2cc(C2(C#N)CC2)c(=O)n3CCCCCCCN2CCC(CC2(C)C)C(F)(F)c2cccc1c2] | 1-((4R)-2,2-difluoro-1²,1²,4-trimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)cyclopropane-1- carbonitrile | 589.3 |
| A44 | 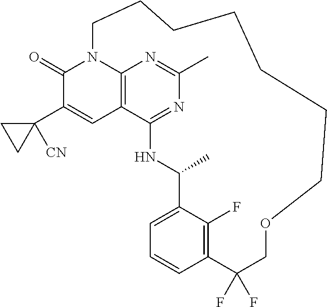 [SMILES: Cc1nc2c3cc(C4(C#N)CC4)c(=O)n(c3n1)CCCCCCCOCC(F)(F)c1cccc(c1F)[C@@H](C)N2] | 1-((3R)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclotetradecaphane- 1⁶-yl)cyclopropane-1-carbonitrile | 540.4 |
| A45 | 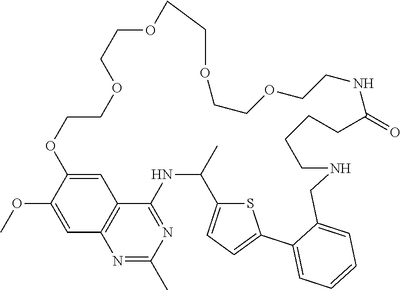 [SMILES: COc1cc2nc(C)nc3c2cc1OCCOCCOCCOCCOCCNC(=O)CCCCNCc1ccccc1-c1ccc(s1)C(C)N3] | 5⁷-methoxy-5²,3-dimethyl-6,9,12,15,18-pentaoxa- 4,21,27-triaza-5(4,6)-quinazolina-2(2,5)- thiophena-1(1,2)-benzenacyclooctacosaphan-22- one | 722.4 |
| A46 | 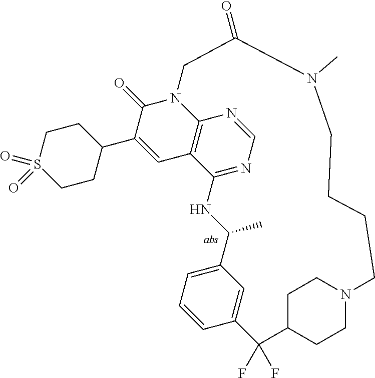 [SMILES: C[C@H]1Nc2ncnc3c2cc(C2CCS(=O)(=O)CC2)c(=O)n3CC(=O)N(C)CCCCN2CCC(CC2)C(F)(F)c2cccc1c2] | (4R)-6⁶-(1,1-dioxidotetrahydro-2H-thiopyran-4- yl)-2,2-difluoro-4,9-dimethyl-6⁷,6⁸-dihydro-5,9- diaza-6(4,8)-pyrido[2,3-d]pyrimidina-1(4,1)- piperidina-3(1,3)-benzenacyclotridecaphane-6⁷,8- dione | 657.3 |
| A47 | 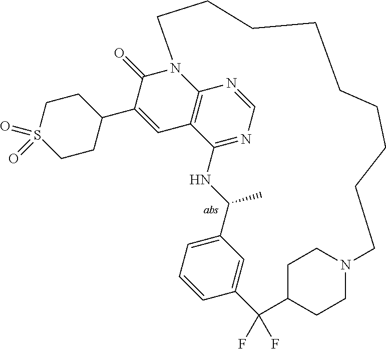 [SMILES: C[C@H]1Nc2ncnc3c2cc(C2CCS(=O)(=O)CC2)c(=O)n3CCCCCCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5-aza- 6(4,8)-pyrido[2,3-d]pyrimidina-1(4,1)-piperidina- 3(1,3)-benzenacyclopentadecaphan-6⁷-one | 656.3 |
| A48 | 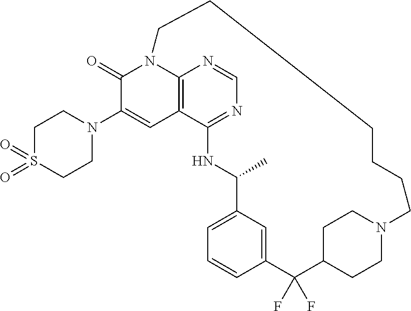 [SMILES: C[C@H]1Nc2ncnc3c2cc(N2CCS(=O)(=O)CC2)c(=O)n3CCCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5-aza- 6(4,8)-pyrido[2,3-d]pyrimidina-1(4,1)-piperidina- 3(1,3)-benzenacyclododecaphan-6⁷-one | 614.4 |
| A49 | 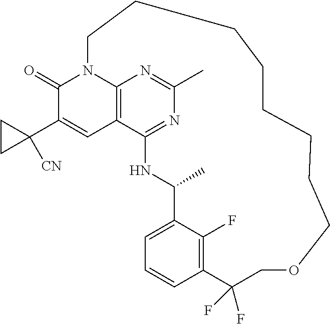 [SMILES: Cc1nc2c3cc(C4(C#N)CC4)c(=O)n(c3n1)CCCCCCCCOCC(F)(F)c1cccc(c1F)[C@@H](C)N2] | 1-((3R)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclopentadecaphane- 1⁶-yl)cyclopropane-1-carbonitrile | 554.3 |
| A50 | 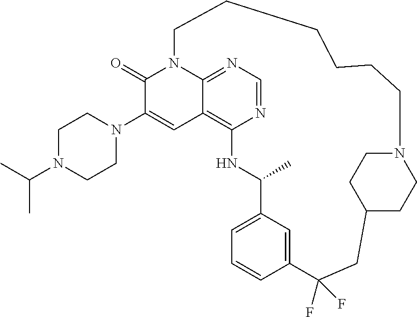 [SMILES: CC(C)N1CCN(c2cc3c4ncnc3n(c2=O)CCCCCCN2CCC(CC2)CC(F)(F)c2cccc(c2)[C@@H](C)N4)CC1] | (3R)-5,5-difluoro-1⁶-(4-isopropylpiperazin-1-yl)-3- methyl-1⁷,1⁸-dihydro-2-aza-1(4,8)-pyrido[2,3- d]pyrimidina-7(4,1)-piperidina-4(1,3)- benzenacyclotridecaphan-1⁷-one | 622.4 |
| A51 | 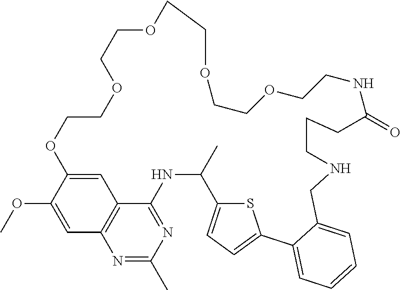 [SMILES: COc1cc2nc(C)nc3c2cc1OCCOCCOCCOCCOCCNC(=O)CCCNCc1ccccc1-c1ccc(s1)C(C)N3] | 5⁷-methoxy-5²,3-dimethyl-6,9,12,15,18-pentaoxa- 4,21,26-triaza-5(4,6)-quinazolina-2(2,5)- thiophena-1(1,2)-benzenacycloheptacosaphan-22- one | 708.3 |
| A52 | 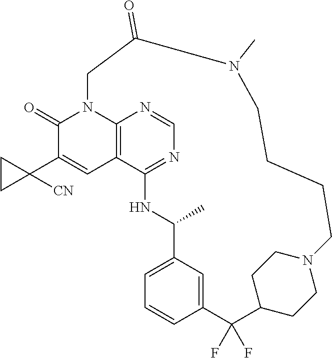 [SMILES: C[C@H]1Nc2ncnc3c2cc(C2(C#N)CC2)c(=O)n3CC(=O)N(C)CCCCN2CCC(CC2)C(F)(F)c2cccc1c2] | 1-((4R)-2,2-difluoro-4,9-dimethyl-6⁷,8-dioxo- 6⁷,6⁸-dihydro-5,9-diaza-6(4,8)-pyrido[2,3- d]pyrimidina-1(4,1)-piperidina-3(1,3)- benzenacyclotridecaphane-6⁶-yl)cyclopropane-1- carbonitrile | 590.3 |
| A53 | 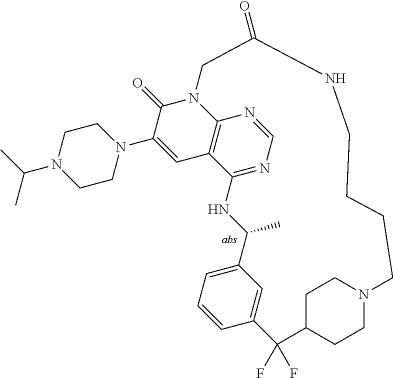 [SMILES: CC(C)N1CCN(c2cc3c4ncnc3n(c2=O)CC(=O)NCCCCN2CCC(CC2)C(F)(F)c2cccc(c2)[C@@H](C)N4)CC1] | (4R)-2,2-difluoro-6⁶-(4-isopropylpiperazin-1-yl)-4- methyl-6⁷,6⁸-dihydro-5,9-diaza-6(4,8)-pyrido[2,3- d]pyrimidina-1(4,1)-piperidina-3(1,3)- benzenacyclotridecaphane-6⁷,8-dione | 637.2 |
| A54 | 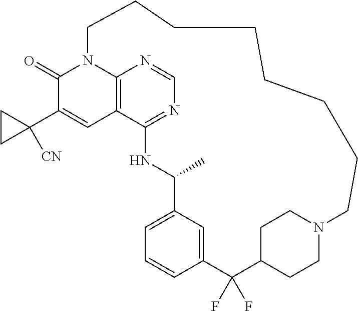 [SMILES: C[C@H]1Nc2ncnc3c2cc(C2(C#N)CC2)c(=O)n3CCCCCCCCN2CCC(CC2)C(F)(F)c2cccc1c2] | 1-((4R)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotetradecaphane-6⁶-yl)cyclopropane-1- carbonitrile | 575.4 |
| A55 | 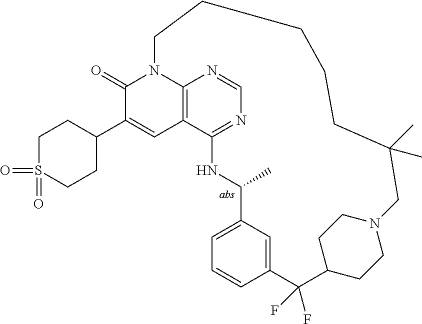 [SMILES: C[C@H]1Nc2ncnc3c2cc(C2CCS(=O)(=O)CC2)c(=O)n3CCCCCC(C)(C)CN2CCC(CC2)C(F)(F)c2cccc1c2] | (4R)-6⁶-(1,1-dioxidotetrahydro-2H-thiopyran-4- yl)-2,2-difluoro-4,12,12-trimethyl-6⁷,6⁸-dihydro-5- aza-6(4,8)-pyrido[2,3-d]pyrimidina-1(4,1)- piperidina-3(1,3)-benzenacyclotridecaphan-6⁷-one | 656.6 |
| A56 | 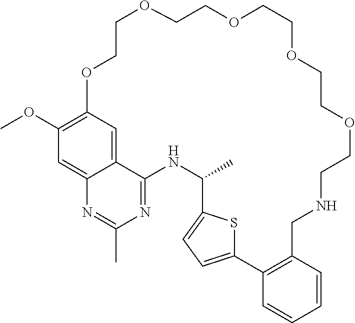 [SMILES: COc1cc2nc(C)nc3c2cc1OCCOCCOCCOCCOCCNCc1ccccc1C1=CC=C(S1)[C@@H](C)N3] | (R)-5⁷-methoxy-5²,3-dimethyl-6,9,12,15,18- pentaoxa-4,21-diaza-5(4,6)-quinazolina-2(2,5)- thiophena-1(1,2)-benzenacyclodocosaphane | 623.2 |
| A57 | 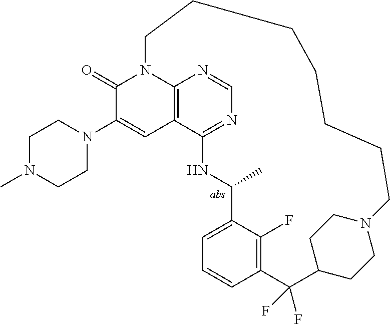 [SMILES: C[C@H]1Nc2ncnc3c2cc(N2CCN(C)CC2)c(=O)n3CCCCCCCN2CCC(CC2)C(F)(F)c2cccc1c2F] | (4R)-3²,2,2-trifluoro-4-methyl-6⁶-(4- methylpiperazin-1-yl)-6⁷,6⁸-dihydro-5-aza-6(4,8)- pyrido[2,3-d]pyrimidina-1(4,1)-piperidina-3(1,3)- benzenacyclotridecaphan-6⁷-one | 612.3 |
| A58 | 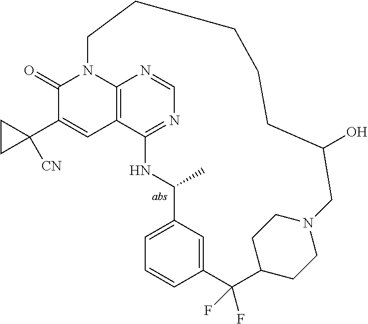 [SMILES: C[C@H]1Nc2ncnc3c2cc(C2(C#N)CC2)c(=O)n3CCCCCC(O)CN2CCC(CC2)C(F)(F)c2cccc1c2] | 1-((4R)-2,2-difluoro-12-hydroxy-4-methyl-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)cyclopropane-1- carbonitrile | 577.3 |
| A59 | 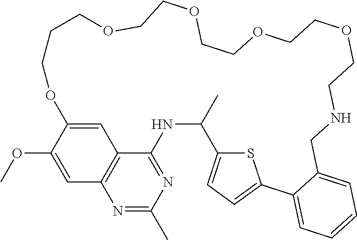 [SMILES: COc1cc2nc(C)nc3c2cc1OCCCOCCOCCOCCOCCNCc1ccccc1-c1ccc(s1)C(C)N3] | 5⁷-methoxy-5²,3-dimethyl-6,10,13,16,19-pentaoxa- 4,22-diaza-5(4,6)-quinazolina-2(2,5)-thiophena- 1(1,2)-benzenacyclotricosaphane | 637.3 |
| A60 | 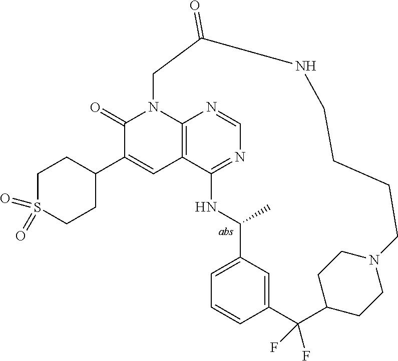 [SMILES: C[C@H]1Nc2ncnc3c2cc(C2CCS(=O)(=O)CC2)c(=O)n3CC(=O)NCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5,9-diaza- 6(4,8)-pyrido[2,3-d]pyrimidina-1(4,1)-piperidina- 3(1,3)-benzenacyclotridecaphane-6⁷,8-dione | 643.3 |
| A61 | 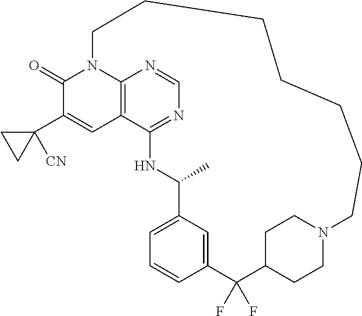 [SMILES: C[C@H]1Nc2ncnc3c2cc(C2(C#N)CC2)c(=O)n3CCCCCCCN2CCC(CC2)C(F)(F)c2cccc1c2] | 1-((4R)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotridecaphane-6⁶-yl)cyclopropane-1- carbonitrile | 561.5 |
| A62 | 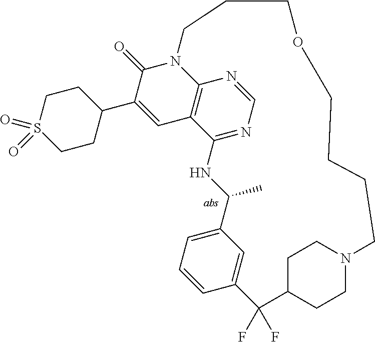 [SMILES: C[C@H]1Nc2ncnc3c2cc(C2CCS(=O)(=O)CC2)c(=O)n3CCCOCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-10-oxa-5- aza-6(4,8)-pyrido[2,3-d]pyrimidina-1(4,1)- piperidina-3(1,3)-benzenacyclotetradecaphan-6⁷- one | 644.5 |
| A63 | 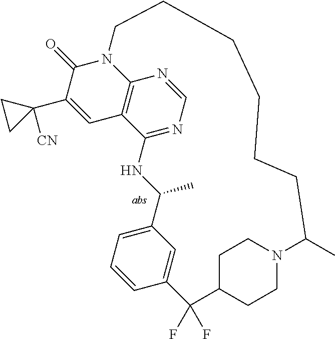 [SMILES: CC1CCCCCCn2c(=O)c(C3(C#N)CC3)cc3c(ncnc32)N[C@H](C)c2cccc(c2)C(F)(F)C2CCN1CC2] | 1-((4R)-2,2-difluoro-4,13-dimethyl-6⁷-oxo-6⁷,6⁸- dihydro-5-aza-6(4,8)-pyrido[2,3-d]pyrimidina- 1(4,1)-piperidina-3(1,3)- benzenacyclotridecaphane-6⁶-yl)cyclopropane-1- carbonitrile | 575.3 |
| A64 | 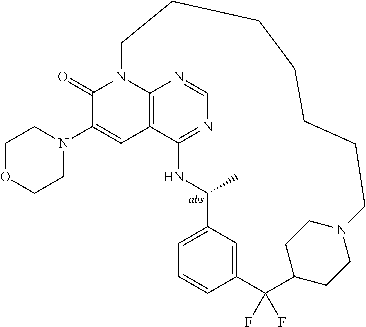 [SMILES: C[C@H]1Nc2ncnc3c2cc(N2CCOCC2)c(=O)n3CCCCCCCN2CCC(CC2)C(F)(F)c2cccc1c2] | (4R)-2,2-difluoro-4-methyl-6⁶-morpholino-6⁷,6⁸- dihydro-5-aza-6(4,8)-pyrido[2,3-d]pyrimidina- 1(4,1)-piperidina-3(1,3)-benzenacyclotridecaphan- 6⁷-one | 581.3 |
| A65 | 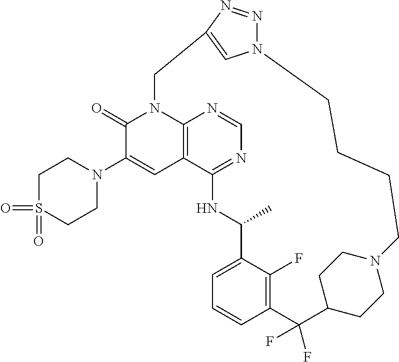 [SMILES: C[C@H]1Nc2ncnc3c2cc(N2CCS(=O)(=O)CC2)c(=O)n3Cc2cn(nn2)CCCCN2CCC(CC2)C(F)(F)c2cccc1c2F] | (5R,Z)-3⁶-(1,1-dioxidothiomorpholino)-6²,7,7- trifluoro-5-methyl-3⁷,3⁸-dihydro-1¹H-4-aza-3(8,4)- pyrido[2,3-d]pyrimidina-8(4,1)-piperidina-1(4,1)- triazola-6(1,3)-benzenacyclododecaphan-3⁷-one | 686.2 |
| A66 | 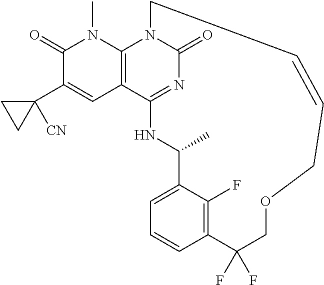 [SMILES: C[C@H]1Nc2nc(=O)n(c3c2cc(C2(C#N)CC2)c(=O)n3C)C/C=C\COCC(F)(F)c2cccc1c2F] | 1-((13E,3R,9Z)-4²,5,5-trifluoro-1⁸,3-dimethyl- 1²,1⁷-dioxo-1¹,1²,1⁷,1⁸-tetrahydro-7-oxa-2-aza- 1(4,1)-pyrido[2,3-d]pyrimidina-4(1,3)- benzenacycloundecaphan-9-en-1⁶- yl)cyclopropane-1-carbonitrile | 512.3 |
| A67 | 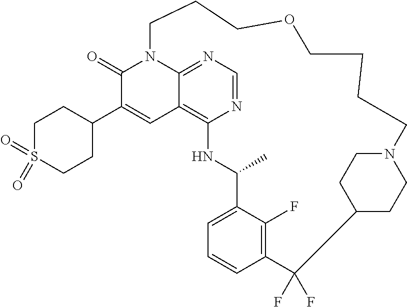 [SMILES: C[C@H]1Nc2ncnc3c2cc(C2CCS(=O)(=O)CC2)c(=O)n3CCCOCCCCN2CCC(CC2)C(F)(F)c2cccc1c2F] | (4R)-6⁶-(4-acetylpiperazin-1-yl)-3²,2,2-trifluoro-4- methyl-6⁷,6⁸-dihydro-5-aza-6(4,8)-pyrido[2,3- d]pyrimidina-1(4,1)-piperidina-3(1,3)- benzenacyclotridecaphan-6⁷-one | 640.4 |
| A68 | 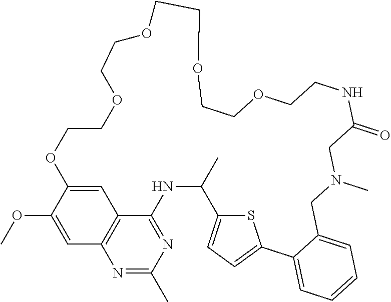 [SMILES: COc1cc2nc(C)nc3c2cc1OCCOCCOCCOCCOCCNC(=O)CN(C)Cc1ccccc1C1=CC=C(S1)C(C)N3] | 5⁷-methoxy-5²,3,24-trimethyl-6,9,1²,15,1⁸- pentaoxa-4,21,24-triaza-5(4,6)-quinazolina-2(2,5)- thiophena-1(1,2)-benzenacyclopentacosaphan-22- one | 694.3 |
| A69 | 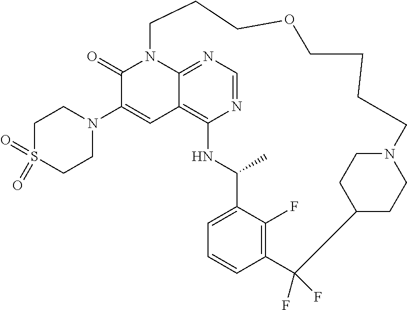 [SMILES: C[C@H]1Nc2ncnc3c2cc(N2CCS(=O)(=O)CC2)c(=O)n3CCCOCCCCN2CCC(CC2)C(F)(F)c2cccc1c2F] | (4R)-6⁶-(1,1-dioxidothiomorpholino)-3²,2,2- trifluoro-4-methyl-6⁷,6⁸-dihydro-10-oxa-5-aza- 6(4,8)-pyrido[2,3-d]pyrimidina-1(4,1)-piperidina- 3(1,3)-benzenacyclotetradecaphan-6⁷-one | 663.3 |
| A70 | 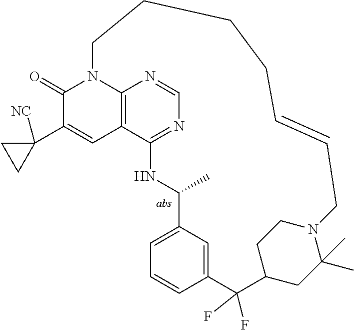 [SMILES: C[C@H]1Nc2ncnc3c2cc(C2(C#N)CC2)c(=O)n3CCCC/C=C/CN2CCC(CC2(C)C)C(F)(F)c2cccc1c2] | 1-((4R,E)-2,2-difluoro-1²,1²,4-trimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphan-11-en-6⁶- yl)cyclopropane-1-carbonitrile | 587.4 |
| A71 | 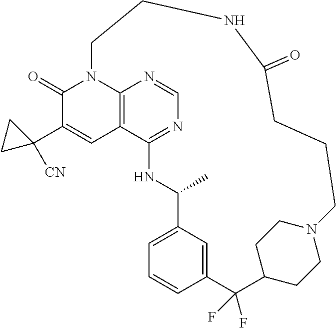 [SMILES: C[C@H]1Nc2ncnc3c2cc(C2(C#N)CC2)c(=O)n3CCNC(=O)CCCN2CCC(CC2)C(F)(F)c2cccc1c2] | 1-((4R)-2,2-difluoro-4-methyl-6⁷,10-dioxo-6⁷,6⁸- dihydro-5,9-diaza-6(4,8)-pyrido[2,3-d]pyrimidina- 1(4,1)-piperidina-3(1,3)- benzenacyclotridecaphane-6⁶-yl)cyclopropane-1- carbonitrile | 576.3 |
| A72 | 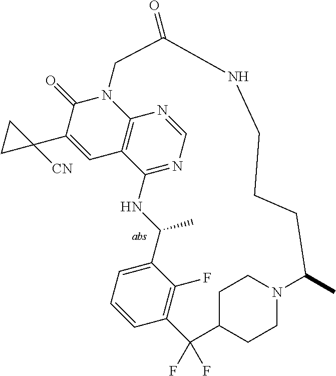 [SMILES: C[C@@H]1CCCNC(=O)Cn2c(=O)c(C3(C#N)CC3)cc3c(ncnc32)N[C@H](C)c2cccc(c2F)C(F)(F)C2CCN1CC2] | 1-((4R,13R)-3²,2,2-trifluoro-4,13-dimethyl-6⁷,8- dioxo-6⁷,6⁸-dihydro-5,9-diaza-6(4,8)-pyrido[2,3- d]pyrimidina-1(4,1)-piperidina-3(1,3)- benzenacyclotridecaphane-6⁶-yl)cyclopropane-1- carbonitrile | 608.3 |
| A73 | 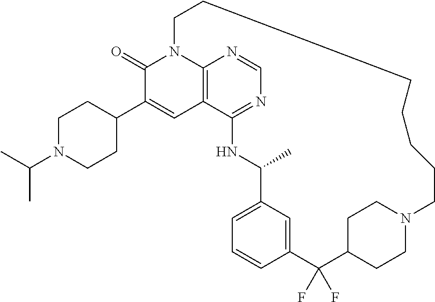 [SMILES: CC(C)N1CCC(c2cc3c4ncnc3n(c2=O)CCCCCCCN2CCC(CC2)C(F)(F)c2cccc(c2)[C@@H](C)N4)CC1] | (4R)-2,2-difluoro-6⁶-(1-isopropylpiperidin-4-yl)-4- methyl-6⁷,6⁸-dihydro-5-aza-6(4,8)-pyrido[2,3- d]pyrimidina-1(4,1)-piperidina-3(1,3)- benzenacyclotridecaphan-6⁷-one | 621.6 |
| A74 | 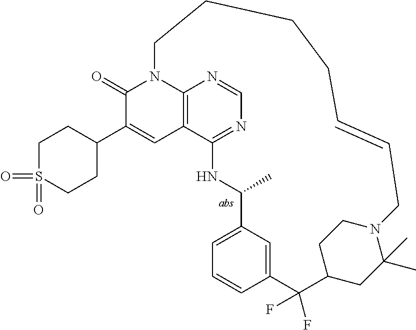 [SMILES: C[C@H]1Nc2ncnc3c2cc(C2CCS(=O)(=O)CC2)c(=O)n3CCCC/C=C/CN2CCC(CC2(C)C)C(F)(F)c2cccc1c2] | (4R,E)-6⁶-(1,1-dioxidotetrahydro-2H-thiopyran-4- yl)-2,2-difluoro-1²,1²,4-trimethyl-6⁷,6⁸-dihydro-5- aza-6(4,8)-pyrido[2,3-d]pyrimidina-1(4,1)- piperidina-3(1,3)-benzenacyclotridecaphan-11-en- 6⁷-one | 654.3 |
| A75 | 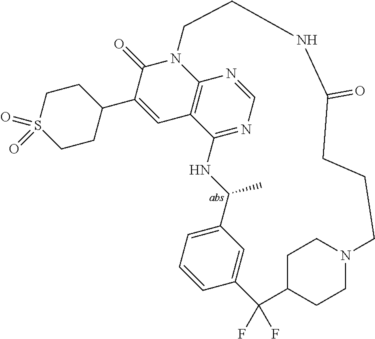 [SMILES: C[C@H]1Nc2ncnc3c2cc(C2CCS(=O)(=O)CC2)c(=O)n3CCNC(=O)CCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5,9-diaza- 6(4,8)-pyrido[2,3-d]pyrimidina-1(4,1)-piperidina- 3(1,3)-benzenacyclotridecaphane-6⁷,10-dione | 643.2 |
| A76 | 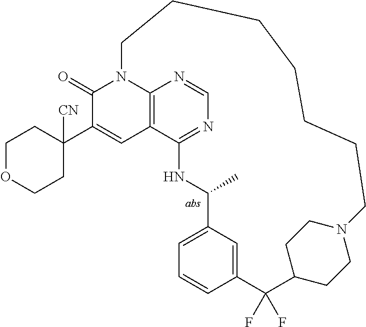 [SMILES: C[C@H]1Nc2ncnc3c2cc(C2(C#N)CCOCC2)c(=O)n3CCCCCCCN2CCC(CC2)C(F)(F)c2cccc1c2] | 4-((4R)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotridecaphane-6⁶-yl)tetrahydro-2H- pyran-4-carbonitrile | 605.2 |
| A77 | 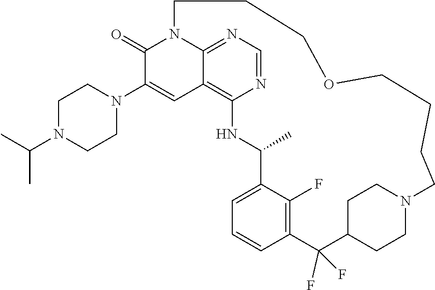 [SMILES: CC(C)N1CCN(c2cc3c4ncnc3n(c2=O)CCCOCCCCN2CCC(CC2)C(F)(F)c2cccc(c2F)[C@@H](C)N4)CC1] | (4R)-3²,2,2-trifluoro-6⁶-(4-isopropylpiperazin-1- yl)-4-methyl-6⁷,6⁸-dihydro-10-oxa-5-aza-6(4,8)- pyrido[2,3-d]pyrimidina-1(4,1)-piperidina-3(1,3)- benzenacyclotetradecaphan-6⁷-one | 656.6 |
| A78 | 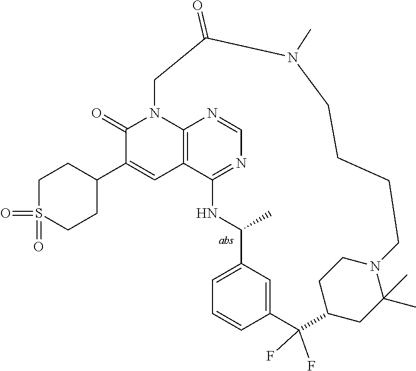 | (1⁴R,4R)-6⁶-(1,1-dioxidotetrahydro-2H-thiopyran- 4-yl)-2,2-difluoro-1²,1²,4,9-tetramethyl-6⁷,6⁸- dihydro-5,9-diaza-6(4,8)-pyrido[2,3-d]pyrimidina- 1(4,1)-piperidina-3(1,3)- benzenacyclotridecaphane-6⁷,8-dione | 685.3 |
| A79 | 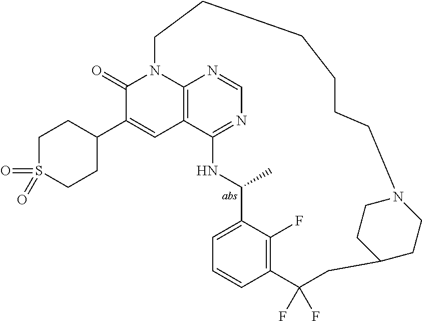 [SMILES: C[C@H]1Nc2ncnc3c2cc(C2CCS(=O)(=O)CC2)c(=O)n3CCCCCCN2CCC(CC2)CC(F)(F)c2cccc1c2F] | (3R)-1⁶-(1,1-dioxidotetrahydro-2H-thiopyran-4- yl)-4²,5,5-trifluoro-3-methyl-1⁷,1⁸-dihydro-2-aza- 1(4,8)-pyrido[2,3-d]pyrimidina-7(4,1)-piperidina- 4(1,3)-benzenacyclotridecaphan-1⁷-one | 646.1 |
| A80 | 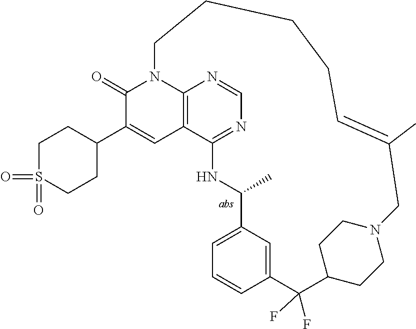 [SMILES: C/C1=C\CCCCn2c(=O)c(C3CCS(=O)(=O)CC3)cc3c(ncnc32)N[C@H](C)c2cccc(c2)C(F)(F)C2CCN(CC2)C1] | (4R,E)-6⁶-(1,1-dioxidotetrahydro-2H-thiopyran-4- yl)-2,2-difluoro-4,12-dimethyl-6⁷,6⁸-dihydro-5- aza-6(4,8)-pyrido[2,3-d]pyrimidina-1(4,1)- piperidina-3(1,3)-benzenacyclotridecaphan-11-en- 6⁷-one | 640.3 |
| A81 | 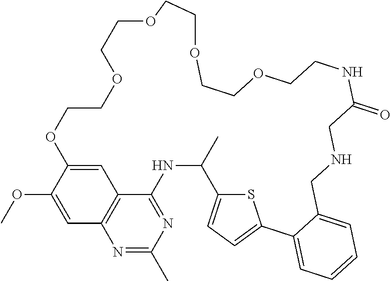 [SMILES: COc1cc2nc(C)nc3c2cc1OCCOCCOCCOCCOCCNC(=O)CNCc1ccccc1C1=CC=C(S1)C(C)N3] | 5⁷-methoxy-5²,3-dimethyl-6,9,12,15,18-pentaoxa- 4,21,24-triaza-5(4,6)-quinazolina-2(2,5)- thiophena-1(1,2)-benzenacyclopentacosaphan-22- one | 680.3 |
| A82 | 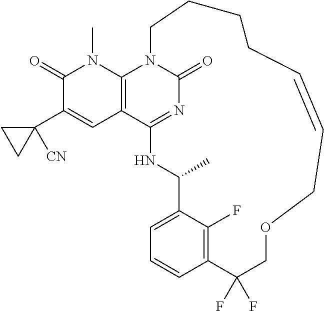 [SMILES: C[C@H]1Nc2nc(=O)n(c3c2cc(C2(C#N)CC2)c(=O)n3C)CCCC/C=C\COCC(F)(F)c2cccc1c2F] | 1-((1³E,3R,9Z)-4²,5,5-trifluoro-1⁸,3-dimethyl- 1²,1⁷-dioxo-1¹,1²,1⁷,1⁸-tetrahydro-7-oxa-2-aza- 1(4,1)-pyrido[2,3-d]pyrimidina-4(1,3)- benzenacyclotetradecaphan-9-en-1⁶- yl)cyclopropane-1-carbonitrile | 554.4 |
| A83 | 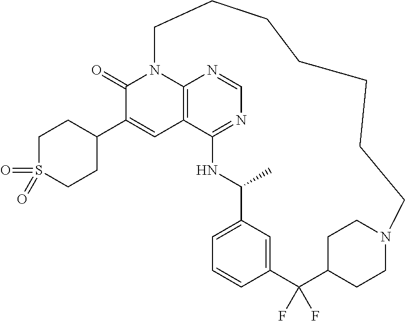 [SMILES: C[C@H]1Nc2ncnc3c2cc(C2CCS(=O)(=O)CC2)c(=O)n3CCCCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5-aza- 6(4,8)-pyrido[2,3-d]pyrimidina-1(4,1)-piperidina- 3(1,3)-benzenacyclotridecaphan-6⁷-one | 628.3 |
| A84 | 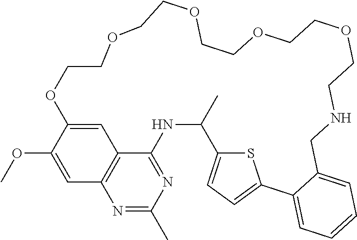 [SMILES: COc1cc2nc(C)nc3c2cc1OCCOCCOCCOCCOCCNCc1ccccc1-c1ccc(s1)C(C)N3] | 5⁷-methoxy-5²,3-dimethyl-6,9,12,15,18-pentaoxa- 4,21-diaza-5(4,6)-quinazolina-2(2,5)-thiophena- 1(1,2)-benzenacyclodocosaphane | 623.3 |
| A85 | 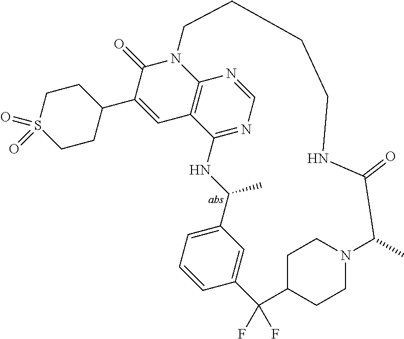 [SMILES: C[C@H]1Nc2ncnc3c2cc(C2CCS(=O)(=O)CC2)c(=O)n3CCCCNC(=O)[C@H](C)N2CCC(CC2)C(F)(F)c2cccc1c2] | (4R,13S)-6⁶-(1,1-dioxidotetrahydro-2H-thiopyran- 4-yl)-2,2-difluoro-4,13-dimethyl-6⁷,6⁸-dihydro- 5,11-diaza-6(4,8)-pyrido[2,3-d]pyrimidina-1(4,1)- piperidina-3(1,3)-benzenacyclotridecaphane-6⁷,12- dione | 657.3 |
| A86 | 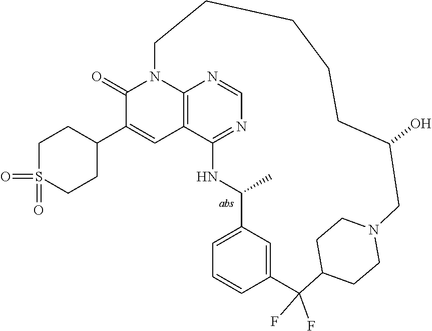 [SMILES: C[C@H]1Nc2ncnc3c2cc(C2CCS(=O)(=O)CC2)c(=O)n3CCCCC[C@H](O)CN2CCC(CC2)C(F)(F)c2cccc1c2] | (4R,12S)-6⁶-(1,1-dioxidotetrahydro-2H-thiopyran- 4-yl)-2,2-difluoro-12-hydroxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 644.3 |
| A87 | 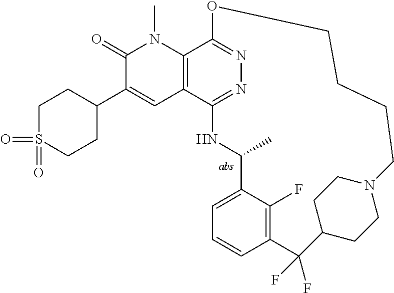 [SMILES: C[C@H]1Nc2nnc(c3c2cc(C2CCS(=O)(=O)CC2)c(=O)n3C)OCCCCN2CCC(CC2)C(F)(F)c2cccc1c2F] | (R)-6³-(1,1-dioxidotetrahydro-2H-thiopyran-4-yl)- 3²,2,2-trifluoro-6¹,4-dimethyl-6¹,6²-dihydro-7-oxa- 5-aza-6(5,8)-pyrido[2,3-d]pyridazina-1(4,1)- piperidina-3(1,3)-benzenacycloundecaphan-6²-one | 634.2 |
| A88 | 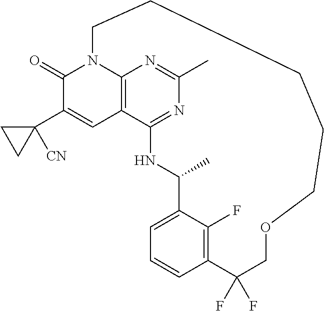 [SMILES: Cc1nc2c3cc(C4(C#N)CC4)c(=O)n(c3n1)CCCCCOCC(F)(F)c1cccc(c1F)[C@@H](C)N2] | 1-((3R)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclododecaphane-1⁶- yl)cyclopropane-1-carbonitrile | 512.4 |
| A89 | 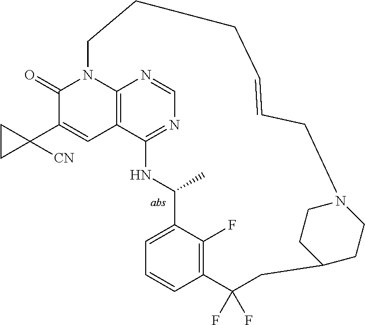 [SMILES: C[C@H]1Nc2ncnc3c2cc(C2(C#N)CC2)c(=O)n3CCC/C=C/CN2CCC(CC2)CC(F)(F)c2cccc1c2F] | 1-((3R,E)-4²,5,5-trifluoro-3-methyl-1⁷-oxo-1⁷,1⁸- dihydro-2-aza-1(4,8)-pyrido[2,3-d]pyrimidina- 7(4,1)-piperidina-4(1,3)-benzenacyclotridecaphan- 9-en-1⁶-yl)cyclopropane-1-carbonitrile | 577.2 |
| A90 | 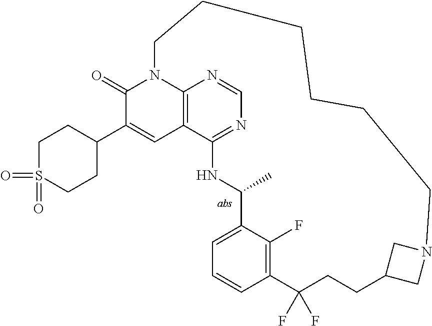 [SMILES: C[C@H]1Nc2ncnc3c2cc(C2CCS(=O)(=O)CC2)c(=O)n3CCCCCCN2CC(CCC(F)(F)c3cccc1c3F)C2] | (3R)-1⁶-(1,1-dioxidotetrahydro-2H-thiopyran-4- yl)-4²,5,5-trifluoro-3-methyl-1⁷,1⁸-dihydro-2-aza- 1(4,8)-pyrido[2,3-d]pyrimidina-8(3,1)-azetidina- 4(1,3)-benzenacyclotetradecaphan-1⁷-one | 632.2 |
| A91 | 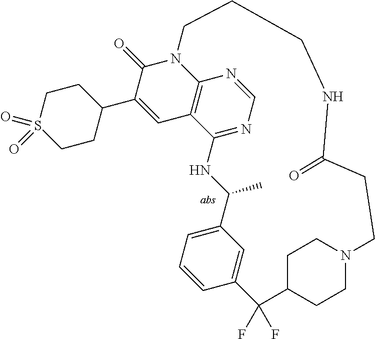 [SMILES: C[C@H]1Nc2ncnc3c2cc(C2CCS(=O)(=O)CC2)c(=O)n3CCCNC(=O)CCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5,10- diaza-6(4,8)-pyrido[2,3-d]pyrimidina-1(4,1)- piperidina-3(1,3)-benzenacyclotridecaphane-6⁷,11- dione | 643.2 |
| A92 | 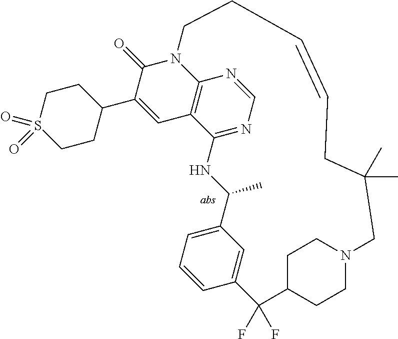 [SMILES: C[C@H]1Nc2ncnc3c2cc(C2CCS(=O)(=O)CC2)c(=O)n3CC/C=C\CC(C)(C)CN2CCC(CC2)C(F)(F)c2cccc1c2] | (4R,Z)-6⁶-(1,1-dioxidotetrahydro-2H-thiopyran-4- yl)-2,2-difluoro-4,12,12-trimethyl-6⁷,6⁸-dihydro-5- aza-6(4,8)-pyrido[2,3-d]pyrimidina-1(4,1)- piperidina-3(1,3)-benzenacyclotridecaphan-9-en- 6⁷-one | 654.4 |
| A93 | 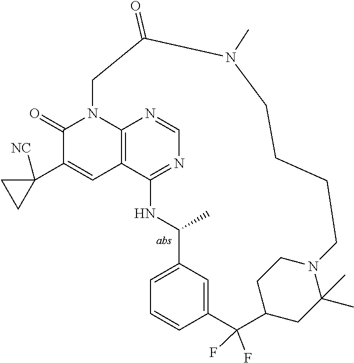 [SMILES: C[C@H]1Nc2ncnc3c2cc(C2(C#N)CC2)c(=O)n3CC(=O)N(C)CCCCN2CCC(CC2(C)C)C(F)(F)c2cccc1c2] | 1-((4R)-2,2-difluoro-1²,1²,4,9-tetramethyl-6⁷,8- dioxo-6⁷,6⁸-dihydro-5,9-diaza-6(4,8)-pyrido[2,3- d]pyrimidina-1(4,1)-piperidina-3(1,3)- benzenacyclotridecaphane-6⁶-yl)cyclopropane-1- carbonitrile | 618.3 |
| A94 | 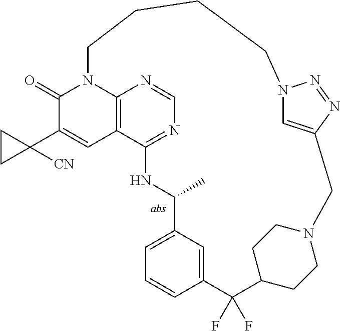 [SMILES: C[C@H]1Nc2ncnc3c2cc(C2(C#N)CC2)c(=O)n3CCCCn2cc(nn2)CN2CCC(CC2)C(F)(F)c2cccc1c2] | 1-((6R,Z)-4,4-difluoro-6-methyl-8⁷-oxo-8⁷,8⁸- dihydro-1¹H-7-aza-8(4,8)-pyrido[2,3- d]pyrimidina-3(1,4)-piperidina-1(4,1)-triazola- 5(1,3)-benzenacyclododecaphane-8⁶- yl)cyclopropane-1-carbonitrile | 600.3 |
| A95 | 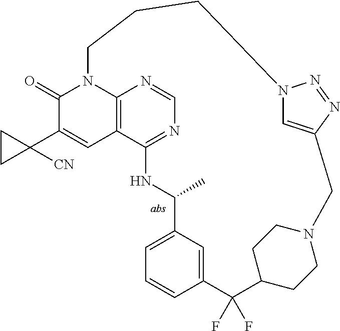 [SMILES: C[C@H]1Nc2ncnc3c2cc(C2(C#N)CC2)c(=O)n3CCCn2cc(nn2)CN2CCC(CC2)C(F)(F)c2cccc1c2] | 1-((6R,Z)-4,4-difluoro-6-methyl-8⁷-oxo-8⁷,8⁸- dihydro-1¹H-7-aza-8(4,8)-pyrido[2,3- d]pyrimidina-3(1,4)-piperidina-1(4,1)-triazola- 5(1,3)-benzenacycloundecaphane-8⁶- yl)cyclopropane-1-carbonitrile | 586.4 |
| A96 | 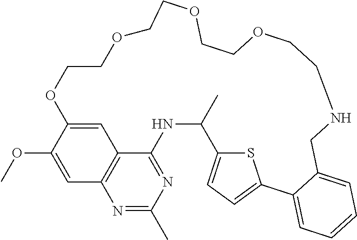 [SMILES: COc1cc2nc(C)nc3c2cc1OCCOCCOCCOCCNCc1ccccc1C1=CC=C(S1)C(C)N3] | 5⁷-methoxy-5²,3-dimethyl-6,9,1²,15-tetraoxa-4,18- diaza-5(4,6)-quinazolina-2(2,5)-thiophena-1(1,2)- benzenacyclononadecaphane | 579.3 |
| A97 | 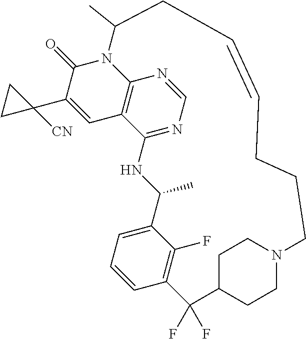 [SMILES: CC1C/C=C\CCCN2CCC(CC2)C(F)(F)c2cccc(c2F)[C@@H](C)Nc2ncnc3c2cc(C2(C#N)CC2)c(=O)n31] | 1-((4R,Z)-3²,2,2-trifluoro-4,7-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphan-9-en-6⁶-yl)cyclopropane- 1-carbonitrile | 591.5 |
| A98 | 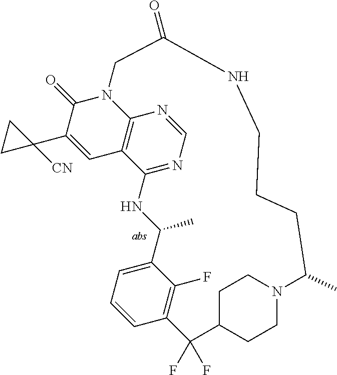 [SMILES: C[C@H]1Nc2ncnc3c2cc(C2(C#N)CC2)c(=O)n3CC(=O)NCCC[C@H](C)N2CCC(CC2)C(F)(F)c2cccc1c2F] | 1-((4R,13S)-3²,2,2-trifluoro-4,13-dimethyl-6⁷,8- dioxo-6⁷,6⁸-dihydro-5,9-diaza-6(4,8)-pyrido[2,3- d]pyrimidina-1(4,1)-piperidina-3(1,3)- benzenacyclotridecaphane-6⁶-yl)cyclopropane-1- carbonitrile | 608.3 |
| A99 | 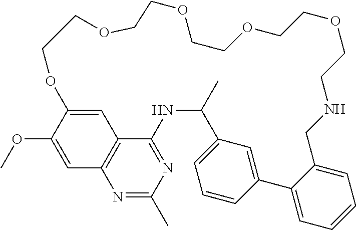 [SMILES: COc1cc2nc(C)nc3c2cc1OCCOCCOCCOCCOCCNCc1ccccc1-c1cccc(c1)C(C)N3] | 5⁷-methoxy-5²,3-dimethyl-6,9,12,15,18-pentaoxa- 4,21-diaza-5(4,6)-quinazolina-1(1,2),2(1,3)- dibenzenacyclodocosaphane | 617.4 |
| A100 | 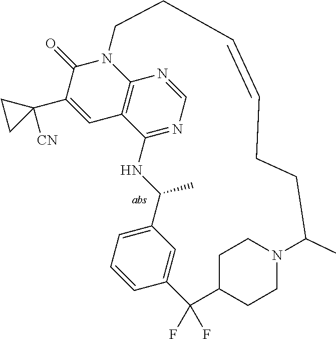 [SMILES: CC1CC/C=C\CCn2c(=O)c(C3(C#N)CC3)cc3c(ncnc32)N[C@H](C)c2cccc(c2)C(F)(F)C2CCN1CC2] | 1-((4R,Z)-2,2-difluoro-4,13-dimethyl-6⁷-oxo-6⁷,6⁸- dihydro-5-aza-6(4,8)-pyrido[2,3-d]pyrimidina- 1(4,1)-piperidina-3(1,3)-benzenacyclotridecaphan- 9-en-6⁶-yl)cyclopropane-1-carbonitrile | 573.3 |
| A101 | 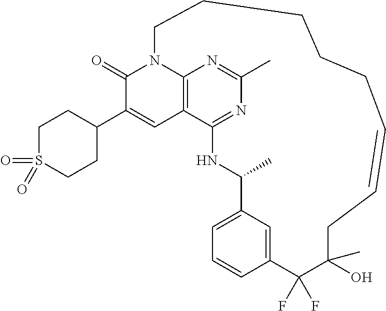 [SMILES: Cc1nc2c3cc(C4CCS(=O)(=O)CC4)c(=O)n(c3n1)CCCCC/C=C\CC(C)(O)C(F)(F)c1cccc(c1)[C@@H](C)N2] | (3R,Z)-1⁶-(1,1-dioxidotetrahydro-2H-thiopyran-4- yl)-5,5-difluoro-6-hydroxy-1²,3,6-trimethyl-1⁷,1⁸- dihydro-2-aza-1(4,8)-pyrido[2,3-d]pyrimidina- 4(1,3)-benzenacyclotetradecaphan-8-en-1⁷-one | 615.4 |
| A102 | 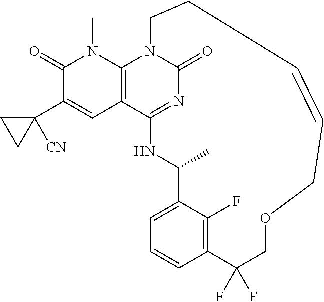 [SMILES: C[C@H]1Nc2nc(=O)n(c3c2cc(C2(C#N)CC2)c(=O)n3C)CC/C=C\COCC(F)(F)c2cccc1c2F] | 1-((1³E,3R,9Z)-4²,5,5-trifluoro-1⁸,3-dimethyl- 1²,1⁷-dioxo-1¹,1²,1⁷,1⁸-tetrahydro-7-oxa-2-aza- 1(4,1)-pyrido[2,3-d]pyrimidina-4(1,3)- benzenacyclododecaphan-9-en-1⁶- yl)cyclopropane-1-carbonitrile | 526.4 |
| A103 | 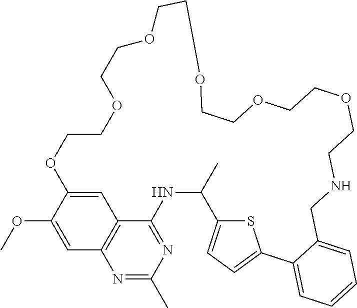 [SMILES: COc1cc2nc(C)nc3c2cc1OCCOCCOCCOCCOCCOCCNCc1ccccc1C1=CC=C(S1)C(C)N3] | 5⁷-methoxy-5²,3-dimethyl-6,9,12,15,18,21- hexaoxa-4,24-diaza-5(4,6)-quinazolina-2(2,5)- thiophena-1(1,2)-benzenacyclopentacosaphane | 667.3 |
| A104 | 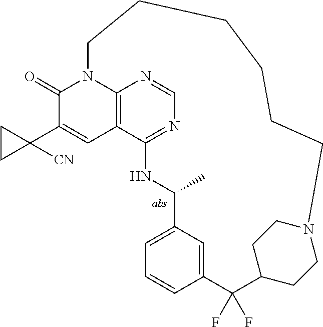 [SMILES: C[C@H]1Nc2ncnc3c2cc(C2(C#N)CC2)c(=O)n3CCCCCCN2CCC(CC2)C(F)(F)c2cccc1c2] | 1-((4R)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclododecaphane-6⁶-yl)cyclopropane-1- carbonitrile | 547.3 |
| A105 | 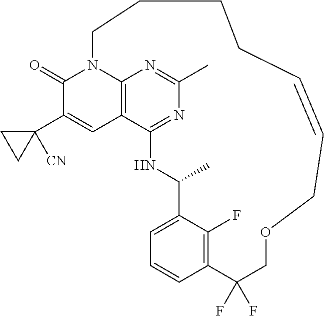 [SMILES: Cc1nc2c3cc(C4(C#N)CC4)c(=O)n(c3n1)CCCC/C=C\COCC(F)(F)c1cccc(c1F)[C@@H](C)N2] | 1-((3R,Z)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclotetradecaphan-9- en-1⁶-yl)cyclopropane-1-carbonitrile | 538.4 |
| A106 | 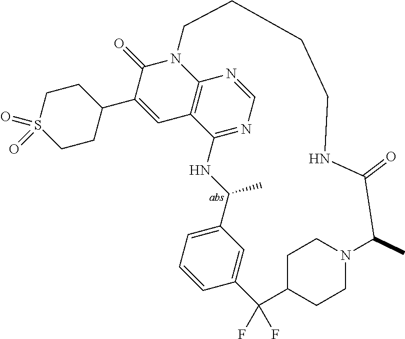 [SMILES: C[C@@H]1C(=O)NCCCCn2c(=O)c(C3CCS(=O)(=O)CC3)cc3c(ncnc32)N[C@H](C)c2cccc(c2)C(F)(F)C2CCN1CC2] | (4R,13R)-6⁶-(1,1-dioxidotetrahydro-2H-thiopyran- 4-yl)-2,2-difluoro-4,13-dimethyl-6⁷,6⁸-dihydro- 5,11-diaza-6(4,8)-pyrido[2,3-d]pyrimidina-1(4,1)- piperidina-3(1,3)-benzenacyclotridecaphane-6⁷,12- dione | 657.3 |
| A107 | 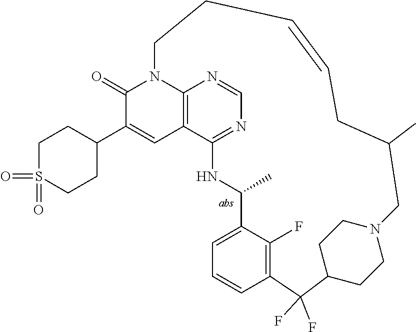 [SMILES: CC1C/C=C\CCn2c(=O)c(C3CCS(=O)(=O)CC3)cc3c(ncnc32)N[C@H](C)c2cccc(c2F)C(F)(F)C2CCN(CC2)C1] | (4R,Z)-6⁶-(1,1-dioxidotetrahydro-2H-thiopyran-4- yl)-3²,2,2-trifluoro-4,12-dimethyl-6⁷,6⁸-dihydro-5- aza-6(4,8)-pyrido[2,3-d]pyrimidina-1(4,1)- piperidina-3(1,3)-benzenacyclotridecaphan-9-en- 6⁷-one | 658.2 |
| A108 | 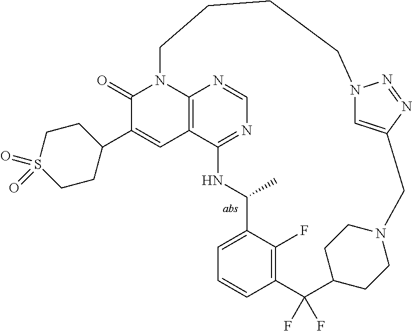 [SMILES: C[C@H]1Nc2ncnc3c2cc(C2CCS(=O)(=O)CC2)c(=O)n3CCCCn2cc(nn2)CN2CCC(CC2)C(F)(F)c2cccc1c2F] | (6R,Z)-8⁶-(1,1-dioxidotetrahydro-2H-thiopyran-4- yl)-5²,4,4-trifluoro-6-methyl-8⁷,8⁸-dihydro-1¹H-7- aza-8(4,8)-pyrido[2,3-d]pyrimidina-3(1,4)- piperidina-1(4,1)-triazola-5(1,3)- benzenacyclododecaphan-8⁷-one | 685.2 |
| A109 | 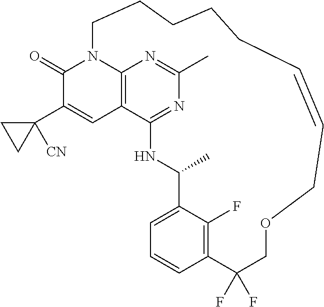 [SMILES: Cc1nc2c3cc(C4(C#N)CC4)c(=O)n(c3n1)CCCCC/C=C\COCC(F)(F)c1cccc(c1F)[C@@H](C)N2] | 1-((3R,Z)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclopentadecaphan- 9-en-1⁶-yl)cyclopropane-1-carbonitrile | 552.4 |
| A110 | 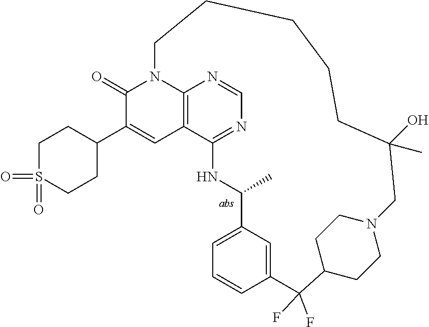 [SMILES: C[C@H]1Nc2ncnc3c2cc(C2CCS(=O)(=O)CC2)c(=O)n3CCCCCC(C)(O)CN2CCC(CC2)C(F)(F)c2cccc1c2] | (4R)-6⁶-(1,1-dioxidotetrahydro-2H-thiopyran-4- yl)-2,2-difluoro-12-hydroxy-4,12-dimethyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 658.3 |
| A111 | 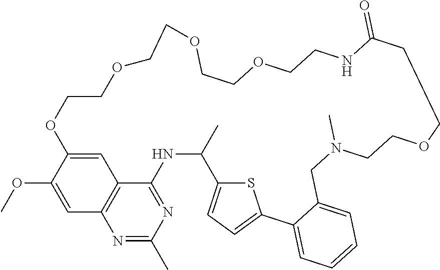 [SMILES: COc1cc2nc(C)nc3c2cc1OCCOCCOCCOCCNC(=O)CCOCCN(C)Cc1ccccc1C1=CC=C(S1)C(C)N3] | 5⁷-methoxy-5²,3,25-trimethyl-6,9,12,15,22- pentaoxa-4,1⁸,25-triaza-5(4,6)-quinazolina-2(2,5)- thiophena-1(1,2)-benzenacyclohexacosaphan-19- one | 708.3 |
| A112 | 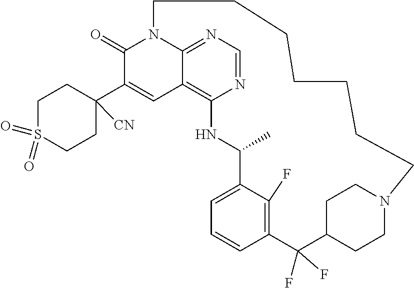 [SMILES: C[C@H]1Nc2ncnc3c2cc(C2(C#N)CCS(=O)(=O)CC2)c(=O)n3CCCCCCCN2CCC(CC2)C(F)(F)c2cccc1c2F] | 4-((4R)-3²,2,2-trifluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotridecaphane-6⁶-yl)tetrahydro-2H- thiopyran-4-carbonitrile 1,1-dioxide | 671.5 |
| A113 | 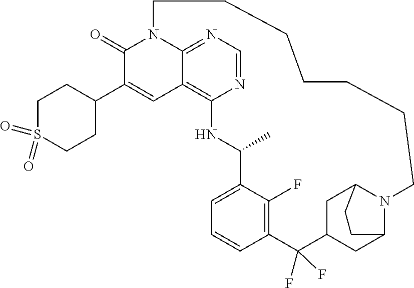 [SMILES: C[C@H]1Nc2ncnc3c2cc(C2CCS(=O)(=O)CC2)c(=O)n3CCCCCCCN2C3CCC2CC(C3)C(F)(F)c2cccc1c2F] | (1¹S,1⁵R,4R)-6⁶-(1,1-dioxidotetrahydro-2H- thiopyran-4-yl)-3²,2,2-trifluoro-4-methyl-6⁷,6⁸ dihydro-1⁸,5-diaza-6(4,8)-pyrido[2,3- d]pyrimidina-1(3,8)-bicyclo[3.2.1]octana-3(1,3)- benzenacyclotridecaphan-6⁷-one | 672.3 |
| A114 | 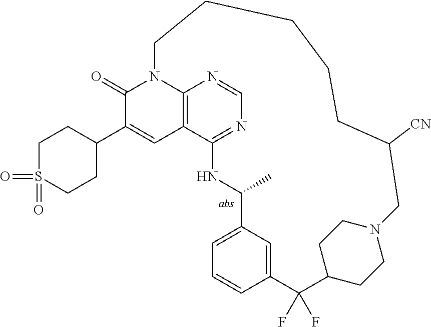 [SMILES: C[C@H]1Nc2ncnc3c2cc(C2CCS(=O)(=O)CC2)c(=O)n3CCCCCC(C#N)CN2CCC(CC2)C(F)(F)c2cccc1c2] | (4R)-6⁶-(1,1-dioxidotetrahydro-2H-thiopyran-4- yl)-2,2-difluoro-4-methyl-6⁷-oxo-6⁷,6⁸-dihydro-5- aza-6(4,8)-pyrido[2,3-d]pyrimidina-1(4,1)- piperidina-3(1,3)-benzenacyclotridecaphane-12- carbonitrile | 653.2 |
| A115 | 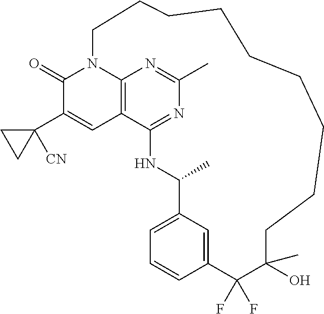 [SMILES: Cc1nc2c3cc(C4(C#N)CC4)c(=O)n(c3n1)CCCCCCCCCC(C)(O)C(F)(F)c1cccc(c1)[C@@H](C)N2] | 1-((3R)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclopentadecaphane- 1⁶-yl)cyclopropane-1-carbonitrile | 564.4 |
| A116 | 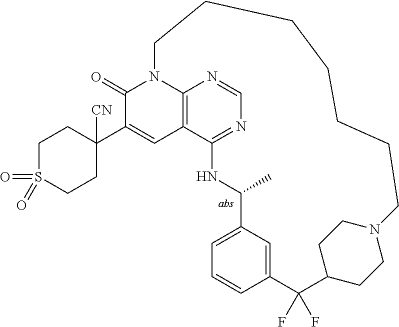 [SMILES: C[C@H]1Nc2ncnc3c2cc(C2(C#N)CCS(=O)(=O)CC2)c(=O)n3CCCCCCCN2CCC(CC2)C(F)(F)c2cccc1c2] | 4-((4R)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotridecaphane-6⁶-yl)tetrahydro-2H- thiopyran-4-carbonitrile 1,1-dioxide | 653.4 |
| A117 | 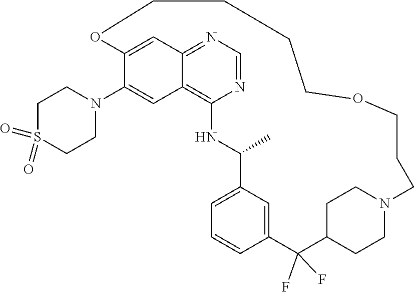 [SMILES: C[C@H]1Nc2ncnc3cc(c(N4CCS(=O)(=O)CC4)cc23)OCCCCOCCCN2CCC(CC2)C(F)(F)c2cccc1c2] | (R)-4-(2,2-difluoro-4-methyl-7,12-dioxa-5-aza- 6(4,7)-quinazolina-1(4,1)-piperidina-3(1,3)- benzenacyclopentadecaphane-6⁶-yl)thiomorpholine 1,1-dioxide | 644.4 |
| A118 | 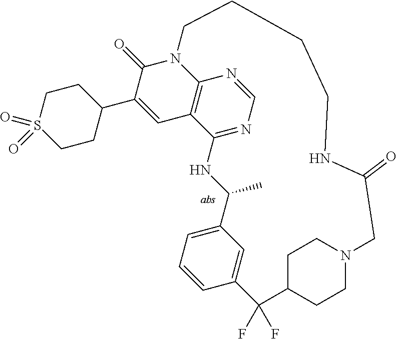 [SMILES: C[C@H]1Nc2ncnc3c2cc(C2CCS(=O)(=O)CC2)c(=O)n3CCCCNC(=O)CN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5,11- diaza-6(4,8)-pyrido[2,3-d]pyrimidina-1(4,1)- piperidina-3(1,3)-benzenacyclotridecaphane-6⁷,12- dione | 643.3 |
| A119 | 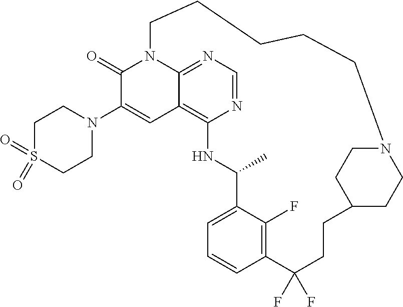 [SMILES: C[C@H]1Nc2ncnc3c2cc(N2CCS(=O)(=O)CC2)c(=O)n3CCCCCN2CCC(CC2)CCC(F)(F)c2cccc1c2F] | (3R)-1⁶-(1,1-dioxidothiomorpholino)-4²,5,5- trifluoro-3-methyl-1⁷,1⁸-dihydro-2-aza-1(4,8)- pyrido[2,3-d]pyrimidina-8(4,1)-piperidina-4(1,3)- benzenacyclotridecaphan-1⁷-one | 647.3 |
| A120 | 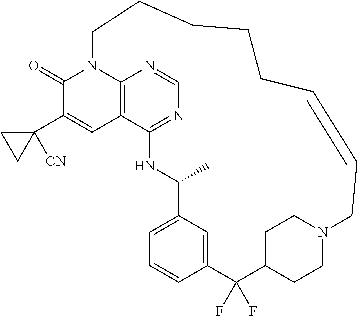 [SMILES: C[C@H]1Nc2ncnc3c2cc(C2(C#N)CC2)c(=O)n3CCCCC/C=C\CN2CCC(CC2)C(F)(F)c2cccc1c2] | 1-((4R,Z)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclotetradecaphan-12-en-6⁶- yl)cyclopropane-1-carbonitrile | 573.4 |
| A121 | 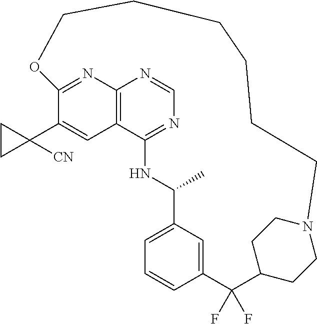 [SMILES: C[C@H]1Nc2ncnc3nc(c(C4(C#N)CC4)cc23)OCCCCCCN2CCC(CC2)C(F)(F)c2cccc1c2] | (R)-1-(2,2-difluoro-4-methyl-7-oxa-5-aza-6(4,7)- pyrido[2,3-d]pyrimidina-1(4,1)-piperidina-3(1,3)- benzenacyclotridecaphane-6⁶-yl)cyclopropane-1- carbonitrile | 547.3 |
| A122 | 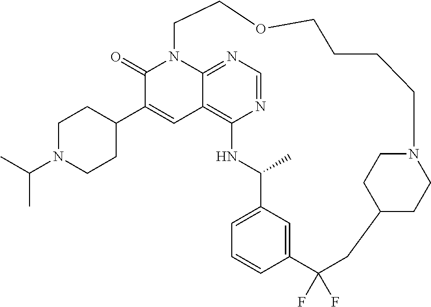 [SMILES: CC(C)N1CCC(c2cc3c4ncnc3n(c2=O)CCOCCCCN2CCC(CC2)CC(F)(F)c2cccc(c2)[C@@H](C)N4)CC1] | (3R)-5,5-difluoro-1⁶-(1-isopropylpiperidin-4-yl)-3- methyl-1⁷,1⁸-dihydro-12-oxa-2-aza-1(4,8)- pyrido[2,3-d]pyrimidina-7(4,1)-piperidina-4(1,3)- benzenacyclotetradecaphan-1⁷-one | 637.5 |
| A123 | 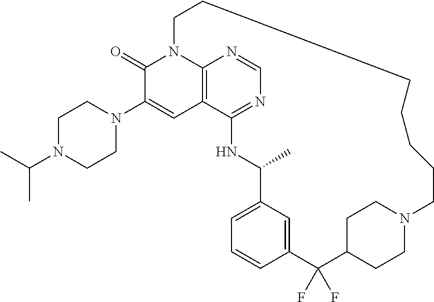 [SMILES: CC(C)N1CCN(c2cc3c4ncnc3n(c2=O)CCCCCCCN2CCC(CC2)C(F)(F)c2cccc(c2)[C@@H](C)N4)CC1] | (4R)-2,2-difluoro-6⁶-(4-isopropylpiperazin-1-yl)-4- methyl-6⁷,6⁸-dihydro-5-aza-6(4,8)-pyrido[2,3- d]pyrimidina-1(4,1)-piperidina-3(1,3)- benzenacyclotridecaphan-6⁷-one | 622.3 |
| A124 | 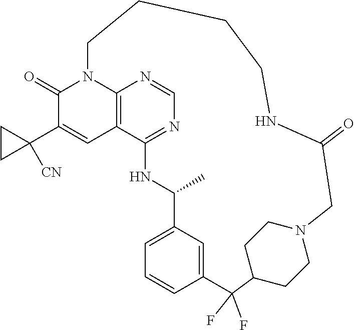 [SMILES: C[C@H]1Nc2ncnc3c2cc(C2(C#N)CC2)c(=O)n3CCCCNC(=O)CN2CCC(CC2)C(F)(F)c2cccc1c2] | 1-((4R)-2,2-difluoro-4-methyl-6⁷,12-dioxo-6⁷,6⁸- dihydro-5,11-diaza-6(4,8)-pyrido[2,3- d]pyrimidina-1(4,1)-piperidina-3(1,3)- benzenacyclotridecaphane-6⁶-yl)cyclopropane-1- carbonitrile | 576.3 |
| A125 | 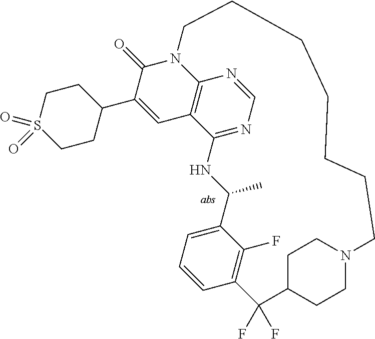 [SMILES: C[C@H]1Nc2ncnc3c2cc(C2CCS(=O)(=O)CC2)c(=O)n3CCCCCCCN2CCC(CC2)C(F)(F)c2cccc1c2F] | (4R)-6⁶-(1,1-dioxidotetrahydro-2H-thiopyran-4- yl)-3²,2,2-trifluoro-4-methyl-6⁷,6⁸-dihydro-5-aza- 6(4,8)-pyrido[2,3-d]pyrimidina-1(4,1)-piperidina- 3(1,3)-benzenacyclotridecaphan-6⁷-one | 646.3 |
| A126 | 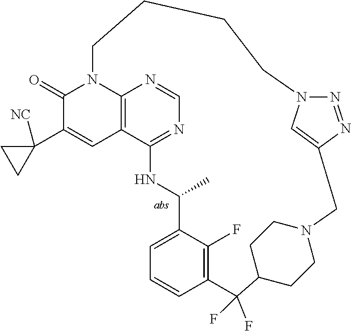 [SMILES: C[C@H]1Nc2ncnc3c2cc(C2(C#N)CC2)c(=O)n3CCCCn2cc(nn2)CN2CCC(CC2)C(F)(F)c2cccc1c2F] | 1-((6R,Z)-5²,4,4-trifluoro-6-methyl-8⁷-oxo-8⁷,8⁸- dihydro-1¹H-7-aza-8(4,8)-pyrido[2,3- d]pyrimidina-3(1,4)-piperidina-1(4,1)-triazola- 5(1,3)-benzenacyclododecaphane-8⁶- yl)cyclopropane-1-carbonitrile | 618.4 |
| A127 | 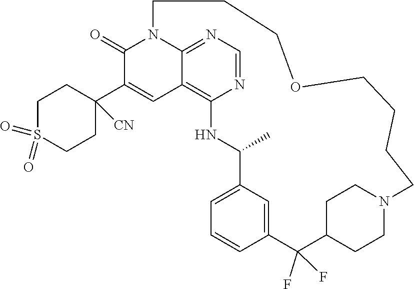 [SMILES: C[C@H]1Nc2ncnc3c2cc(C2(C#N)CCS(=O)(=O)CC2)c(=O)n3CCCOCCCCN2CCC(CC2)C(F)(F)c2cccc1c2] | 4-((4R)-2,2-difluoro-4-methyl-6⁷-oxo-6⁷,6⁸- dihydro-10-oxa-5-aza-6(4,8)-pyrido[2,3- d]pyrimidina-1(4,1)-piperidina-3(1,3)- benzenacyclotetradecaphane-6⁶-yl)tetrahydro-2H- thiopyran-4-carbonitrile 1,1-dioxide | 669.3 |
| A128 | 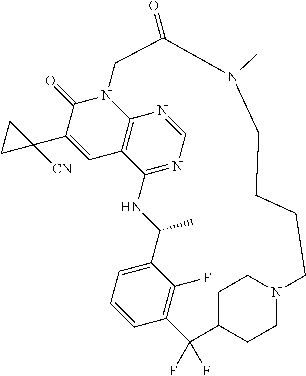 [SMILES: C[C@H]1Nc2ncnc3c2cc(C2(C#N)CC2)c(=O)n3CC(=O)N(C)CCCCN2CCC(CC2)C(F)(F)c2cccc1c2F] | 1-((4R)-3²,2,2-trifluoro-4,9-dimethyl-6⁷,8-dioxo- 6⁷,6⁸-dihydro-5,9-diaza-6(4,8)-pyrido[2,3- d]pyrimidina-1(4,1)-piperidina-3(1,3)- benzenacyclotridecaphane-6⁶-yl)cyclopropane-1- carbonitrile | 608.4 |
| A129 | 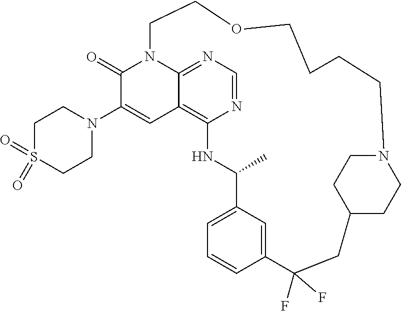 [SMILES: C[C@H]1Nc2ncnc3c2cc(N2CCS(=O)(=O)CC2)c(=O)n3CCOCCCCN2CCC(CC2)CC(F)(F)c2cccc1c2] | (3R)-1⁶-(1,1-dioxidothiomorpholino)-5,5-difluoro- 3-methyl-1⁷,1⁸-dihydro-12-oxa-2-aza-1(4,8)- pyrido[2,3-d]pyrimidina-7(4,1)-piperidina-4(1,3)- benzenacyclotetradecaphan-1⁷-one | 645.4 |
| A130 | 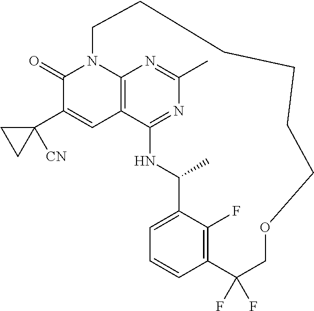 [SMILES: Cc1nc2c3cc(C4(C#N)CC4)c(=O)n(c3n1)CCCCCCOCC(F)(F)c1cccc(c1F)[C@@H](C)N2] | 1-((3R)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclotridecaphane-1⁶- yl)cyclopropane-1-carbonitrile | 526.4 |
| A131 | 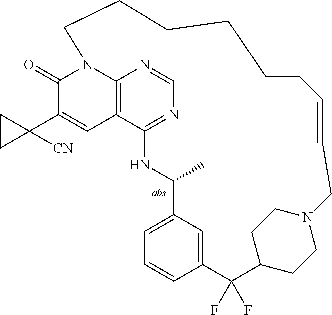 [SMILES: C[C@H]1Nc2ncnc3c2cc(C2(C#N)CC2)c(=O)n3CCCCCC/C=C/CN2CCC(CC2)C(F)(F)c2cccc1c2] | 1-((4R,E)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)- benzenacyclopentadecaphan-13-en-6⁶- yl)cyclopropane-1-carbonitrile | 587.3 |
| A132 | 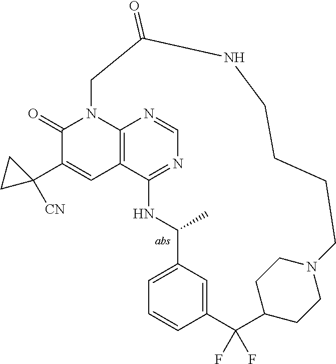 [SMILES: C[C@H]1Nc2ncnc3c2cc(C2(C#N)CC2)c(=O)n3CC(=O)NCCCCN2CCC(CC2)C(F)(F)c2cccc1c2] | 1-((4R)-2,2-difluoro-4-methyl-6⁷,8-dioxo-6⁷,6⁸- dihydro-5,9-diaza-6(4,8)-pyrido[2,3-d]pyrimidina- 1(4,1)-piperidina-3(1,3)- benzenacyclotridecaphane-6⁶-yl)cyclopropane-1- carbonitrile | 576.7 |
| A133 | 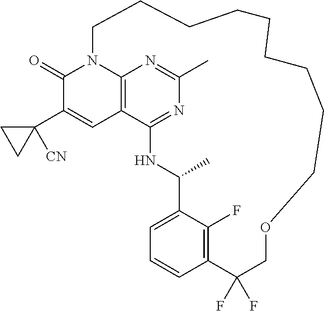 [SMILES: Cc1nc2c3cc(C4(C#N)CC4)c(=O)n(c3n1)CCCCCCCCCOCC(F)(F)c1cccc(c1F)[C@@H](C)N2] | 1-((3R)-4²,5,5-trifluoro-1²,3-dimethyl-1⁷-oxo- 1⁷,1⁸-dihydro-7-oxa-2-aza-1(4,8)-pyrido[2,3- d]pyrimidina-4(1,3)-benzenacyclohexadecaphane- 1⁶-yl)cyclopropane-1-carbonitrile | 568.5 |
| A134 | 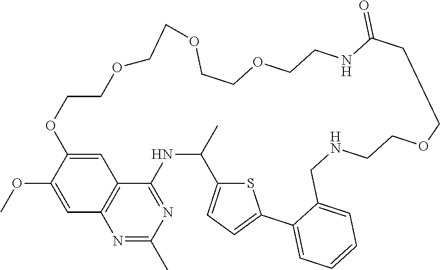 [SMILES: COc1cc2nc(C)nc3c2cc1OCCOCCOCCOCCNC(=O)CCOCCNCc1ccccc1C1=CC=C(S1)C(C)N3] | 5⁷-methoxy-5²,3-dimethyl-6,9,12,15,22-pentaoxa- 4,1⁸,25-triaza-5(4,6)-quinazolina-2(2,5)- thiophena-1(1,2)-benzenacyclohexacosaphan-19- one | 694.3 |
| A135 | 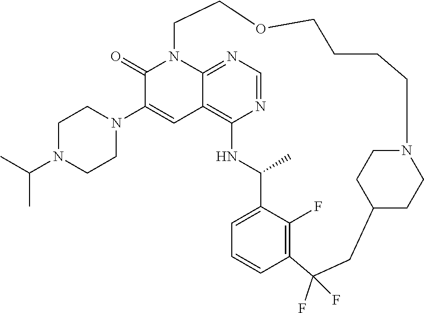 [SMILES: CC(C)N1CCN(c2cc3c4ncnc3n(c2=O)CCOCCCCN2CCC(CC2)CC(F)(F)c2cccc(c2F)[C@@H](C)N4)CC1] | (3R)-4²,5,5-trifluoro-1⁶-(4-isopropylpiperazin-1- yl)-3-methyl-1⁷,1⁸-dihydro-12-oxa-2-aza-1(4,8)- pyrido[2,3-d]pyrimidina-7(4,1)-piperidina-4(1,3)- benzenacyclotetradecaphan-1⁷-one | 656.7 |
| A136 | 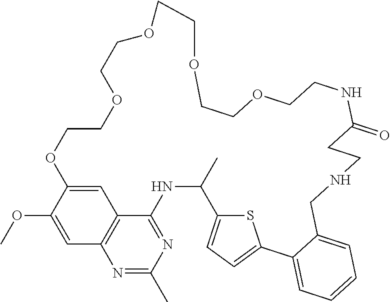 [SMILES: COc1cc2nc(C)nc3c2cc1OCCOCCOCCOCCOCCNC(=O)CCNCc1ccccc1C1=CC=C(S1)C(C)N3] | 5⁷-methoxy-5²,3-dimethyl-6,9,12,15,18-pentaoxa- 4,21,25-triaza-5(4,6)-quinazolina-2(2,5)- thiophena-1(1,2)-benzenacyclohexacosaphan-22- one | 694.3 |
| A137 | 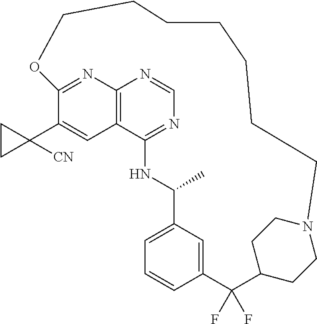 [SMILES: C[C@H]1Nc2ncnc3nc(c(C4(C#N)CC4)cc23)OCCCCCCCN2CCC(CC2)C(F)(F)c2cccc1c2] | (R)-1-(2,2-difluoro-4-methyl-7-oxa-5-aza-6(4,7)- pyrido[2,3-d]pyrimidina-1(4,1)-piperidina-3(1,3)- benzenacyclotetradecaphane-6⁶-yl)cyclopropane-1- carbonitrile | 561.4 |
| A138 | 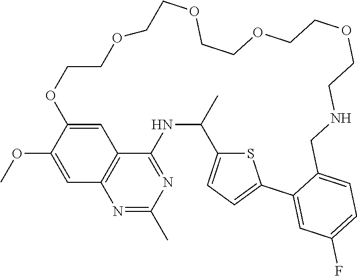 [SMILES: COc1cc2nc(C)nc3c2cc1OCCOCCOCCOCCOCCNCc1ccc(F)cc1C1=CC=C(S1)C(C)N3] | 1⁵-fluoro-5⁷-methoxy-5²,3-dimethyl-6,9,12,15,18- pentaoxa-4,21-diaza-5(4,6)-quinazolina-2(2,5)- thiophena-1(1,2)-benzenacyclodocosaphane | 641.3 |
| A139 | 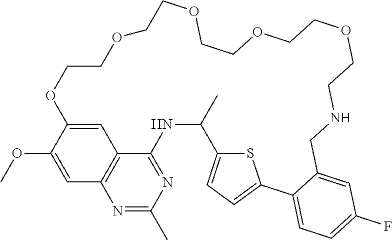 [SMILES: COc1cc2nc(C)nc3c2cc1OCCOCCOCCOCCOCCNCc1cc(F)ccc1C1=CC=C(S1)C(C)N3] | 1⁴-fluoro-5⁷-methoxy-5²,3-dimethyl-6,9,12,15,18- pentaoxa-4,21-diaza-5(4,6)-quinazolina-2(2,5)- thiophena-1(1,2)-benzenacyclodocosaphane | 641.3 |
| A140 | 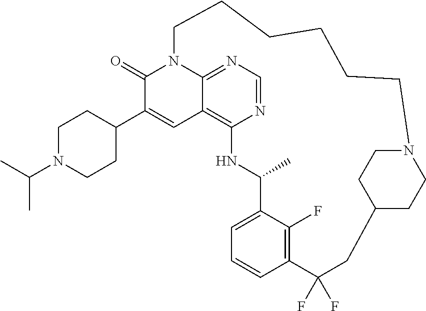 [SMILES: CC(C)N1CCC(c2cc3c4ncnc3n(c2=O)CCCCCCN2CCC(CC2)CC(F)(F)c2cccc(c2F)[C@@H](C)N4)CC1] | (3R)-4²,5,5-trifluoro-1⁶-(1-isopropylpiperidin-4- yl)-3-methyl-1⁷,1⁸-dihydro-2-aza-1(4,8)- pyrido[2,3-d]pyrimidina-7(4,1)-piperidina-4(1,3)- benzenacyclotridecaphan-1⁷-one | 639.3 |
| A141 | 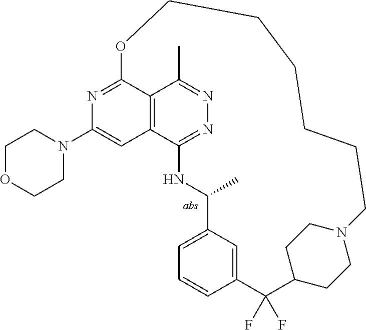 [SMILES: Cc1nnc2c3cc(N4CCOCC4)nc(c13)OCCCCCCN1CCC(CC1)C(F)(F)c1cccc(c1)[C@@H](C)N2] | (R)-2,2-difluoro-6⁴,4-dimethyl-6⁷-morpholino-7- oxa-5-aza-6(1,5)-pyrido[3,4-d]pyridazina-1(4,1)- piperidina-3(1,3)-benzenacyclotridecaphane | 581.2 |
| A142 | 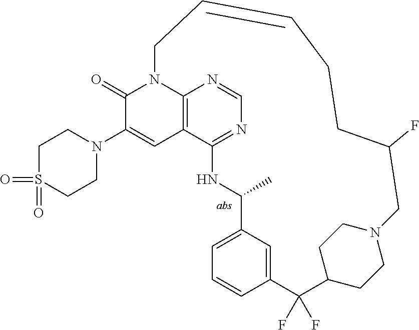 [SMILES: C[C@H]1Nc2ncnc3c2cc(N2CCS(=O)(=O)CC2)c(=O)n3C/C=C\CCC(F)CN2CCC(CC2)C(F)(F)c2cccc1c2] | (4R,Z)-6⁶-(1,1-dioxidotetrahydro-2H-thiopyran-4- yl)-2,2,12-trifluoro-4-methyl-6⁷,6⁸-dihydro-5-aza- 6(4,8)-pyrido[2,3-d]pyrimidina-1(4,1)-piperidina- 3(1,3)-benzenacyclotridecaphan-8-en-6⁷-one | 644.2 |
| A143 | 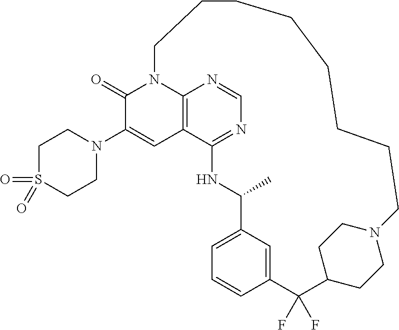 [SMILES: C[C@H]1Nc2ncnc3c2cc(N2CCS(=O)(=O)CC2)c(=O)n3CCCCCCCCN2CCC(CC2)C(F)(F)c2cccc1c2] | (4R)-6⁶-(1,1-dioxidotetrahydro-2H-thiopyran-4- yl)-2,2-difluoro-4-methyl-6⁷,6⁸-dihydro-5-aza- 6(4,8)-pyrido[2,3-d]pyrimidina-1(4,1)-piperidina- 3(1,3)-benzenacyclotetradecaphan-6⁷-one | 642.4 |
| A144 | 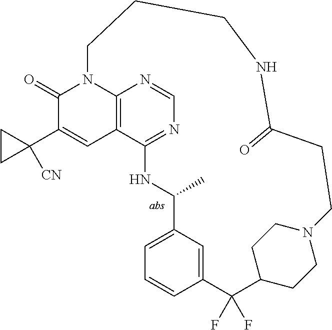 [SMILES: C[C@H]1Nc2ncnc3c2cc(C2(C#N)CC2)c(=O)n3CCCNC(=O)CCN2CCC(CC2)C(F)(F)c2cccc1c2] | 1-((4R)-2,2-difluoro-4-methyl-6⁷,11-dioxo-6⁷,6⁸- dihydro-5,10-diaza-6(4,8)-pyrido[2,3- d]pyrimidina-1(4,1)-piperidina-3(1,3)- benzenacyclotridecaphane-6⁶-yl)cyclopropane-1- carbonitrile | 576.3 |
| A145 | 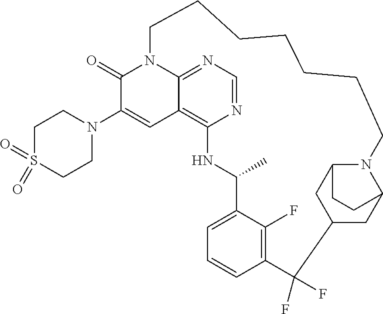 [SMILES: C[C@H]1Nc2ncnc3c2cc(N2CCS(=O)(=O)CC2)c(=O)n3CCCCCCCN2C3CCC2CC(C3)C(F)(F)c2cccc1c2F] | (1¹S,1⁵R,4R)-6⁶-(1,1-dioxidotetrahydro-2H- thiopyran-4-yl)-3²,2,2-trifluoro-4-methyl-6⁷,6⁸- dihydro-1⁸,5-diaza-6(4,8)-pyrido[2,3- d]pyrimidina-1(3,8)-bicyclo[3.2.1]octana-3(1,3)- benzenacyclotridecaphan-6⁷-one | 672.3 |
| A146 | 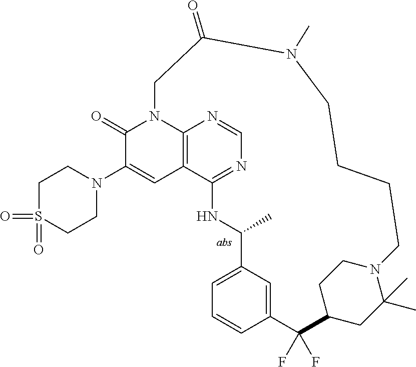 | (1⁴S,4R)-6⁶-(1,1-dioxidotetrahydro-2H-thiopyran- 4-yl)-2,2-difluoro-1²,1²,4,9-tetramethyl-6⁷,6⁸- dihydro-5,9-diaza-6(4,8)-pyrido[2,3-d]pyrimidina- 1(4,1)-piperidina-3(1,3)- benzenacyclotridecaphane-6⁷,8-dione | 685.3 |
| A147 | 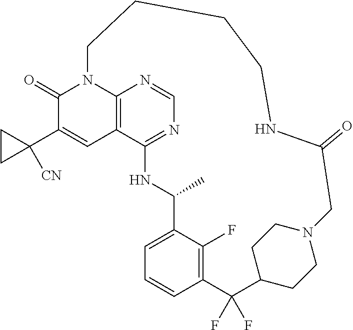 [SMILES: C[C@H]1Nc2ncnc3c2cc(C2(C#N)CC2)c(=O)n3CCCCNC(=O)CN2CCC(CC2)C(F)(F)c2cccc1c2F] | 1-((4R)-3²,2,2-trifluoro-4-methyl-6⁷,12-dioxo- 6⁷,6⁸-dihydro-5,11-diaza-6(4,8)-pyrido[2,3- d]pyrimidina-1(4,1)-piperidina-3(1,3)- benzenacyclotridecaphane-6⁶-yl)cyclopropane-1- carbonitrile | 594.3 |
| A148 | 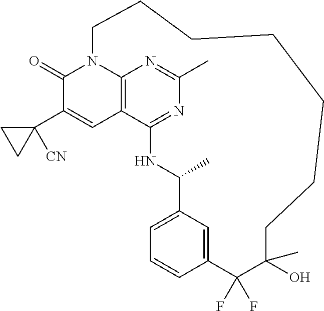 [SMILES: Cc1nc2c3cc(C4(C#N)CC4)c(=O)n(c3n1)CCCCCCCC(C)(O)C(F)(F)c1cccc(c1)[C@@H](C)N2] | 1-((3R)-5,5-difluoro-6-hydroxy-1²,3,6-trimethyl- 1⁷-oxo-1⁷,1⁸-dihydro-2-aza-1(4,8)-pyrido[2,3- d]pyrimidina-4(1,3)-benzenacyclotridecaphane-1⁶- yl)cyclopropane-1-carbonitrile | 536.4 |
| A149 | 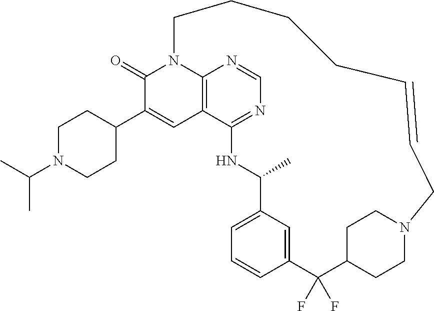 [SMILES: CC(C)N1CCC(c2cc3c4ncnc3n(c2=O)CCCC/C=C/CN2CCC(CC2)C(F)(F)c2cccc(c2)[C@@H](C)N4)CC1] | (4R,E)-2,2-difluoro-6⁶-(1-isopropylpiperidin-4-yl)- 4-methyl-6⁷,6⁸-dihydro-5-aza-6(4,8)-pyrido[2,3- d]pyrimidina-1(4,1)-piperidina-3(1,3)- benzenacyclotridecaphan-11-en-6⁷-one | 619.4 |
| A150 | 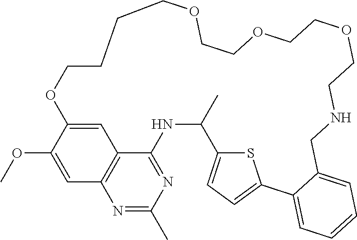 [SMILES: COc1cc2nc(C)nc3c2cc1OCCCCOCCOCCOCCNCc1ccccc1C1=CC=C(S1)C(C)N3] | 5⁷-methoxy-5²,3-dimethyl-6,11,14,17-tetraoxa- 4,20-diaza-5(4,6)-quinazolina-2(2,5)-thiophena- 1(1,2)-benzenacyclohenicosaphane | 607.3 |
| A151 | 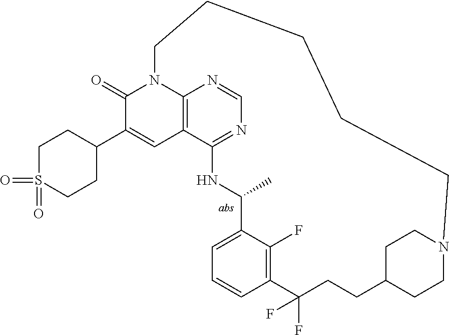 [SMILES: C[C@H]1Nc2ncnc3c2cc(C2CCS(=O)(=O)CC2)c(=O)n3CCCCCN2CCC(CC2)CCC(F)(F)c2cccc1c2F] | (3R)-1⁶-(1,1-dioxidotetrahydro-2H-thiopyran-4- yl)-4²,5,5-trifluoro-3-methyl-1⁷,1⁸-dihydro-2-aza- 1(4,8)-pyrido[2,3-d]pyrimidina-8(4,1)-piperidina- 4(1,3)-benzenacyclotridecaphan-1⁷-one | 646.2 |
| A152 | 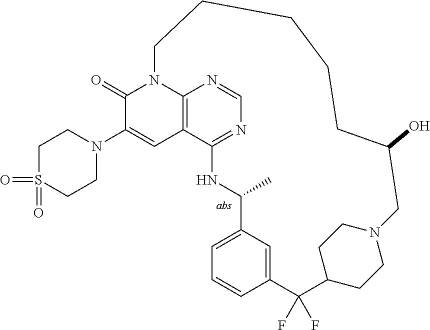 [SMILES: C[C@H]1Nc2ncnc3c2cc(N2CCS(=O)(=O)CC2)c(=O)n3CCCCC[C@@H](O)CN2CCC(CC2)C(F)(F)c2cccc1c2] | (4R,12R)-6⁶-(1,1-dioxidotetrahydro-2H-thiopyran- 4-yl)-2,2-difluoro-12-hydroxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 644.2 |
| A153 | 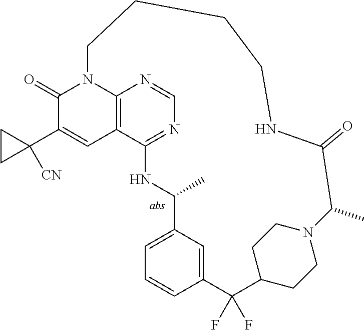 [SMILES: C[C@H]1Nc2ncnc3c2cc(C2(C#N)CC2)c(=O)n3CCCCNC(=O)[C@H](C)N2CCC(CC2)C(F)(F)c2cccc1c2] | 1-((4R,13S)-2,2-difluoro-4,13-dimethyl-6⁷,12- dioxo-6⁷,6⁸-dihydro-5,11-diaza-6(4,8)-pyrido[2,3- d]pyrimidina-1(4,1)-piperidina-3(1,3)- benzenacyclotridecaphane-6⁶-yl)cyclopropane-1- carbonitrile | 590.3 |
| A154 | 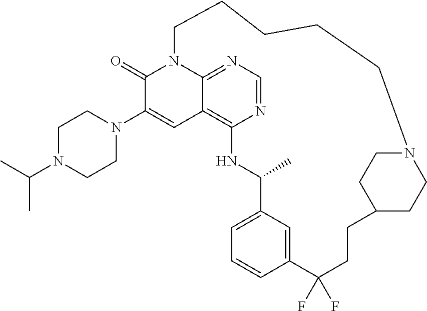 [SMILES: CC(C)N1CCN(c2cc3c4ncnc3n(c2=O)CCCCCN2CCC(CC2)CCC(F)(F)c2cccc(c2)[C@@H](C)N4)CC1] | (3R)-5,5-difluoro-1⁶-(4-isopropylpiperazin-1-yl)-3- methyl-1⁷,1⁸-dihydro-2-aza-1(4,8)-pyrido[2,3- d]pyrimidina-8(4,1)-piperidina-4(1,3)- benzenacyclotridecaphan-1⁷-one | 622.4 |
| A155 | 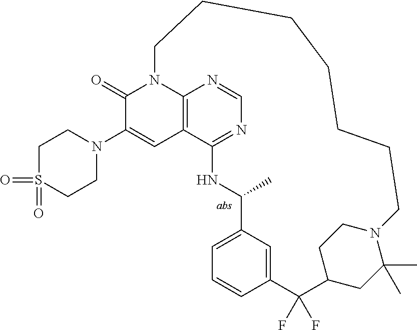 [SMILES: C[C@H]1Nc2ncnc3c2cc(N2CCS(=O)(=O)CC2)c(=O)n3CCCCCCCN2CCC(CC2(C)C)C(F)(F)c2cccc1c2] | (4R)-6⁶-(1,1-dioxidotetrahydro-2H-thiopyran-4- yl)-2,2-difluoro-1²,1²,4-trimethyl-6⁷,6⁸-dihydro-5- aza-6(4,8)-pyrido[2,3-d]pyrimidina-1(4,1)- piperidina-3(1,3)-benzenacyclotridecaphan-6⁷-one | 656.3 |
| A156 | 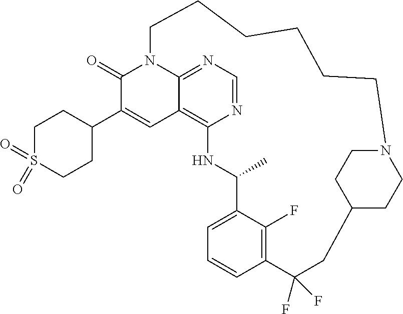 [SMILES: C[C@H]1Nc2ncnc3c2cc(C2CCS(=O)(=O)CC2)c(=O)n3CCCCCCN2CCC(CC2)CC(F)(F)c2cccc1c2F] | (3R)-1⁶-(1,1-dioxidothiomorpholino)-4²,5,5- trifluoro-3-methyl-1⁷,1⁸-dihydro-2-aza-1(4,8)- pyrido[2,3-d]pyrimidina-7(4,1)-piperidina-4(1,3)- benzenacyclotridecaphan-1⁷-one | 646.8 |
| A157 | 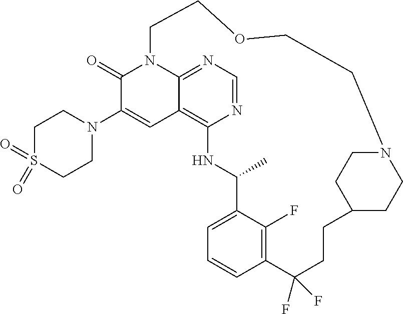 [SMILES: C[C@H]1Nc2ncnc3c2cc(N2CCS(=O)(=O)CC2)c(=O)n3CCOCCN2CCC(CC2)CCC(F)(F)c2cccc1c2F] | (3R)-1⁶-(1,1-dioxidotetrahydro-2H-thiopyran-4- yl)-4²,5,5-trifluoro-3-methyl-1⁷,1⁸-dihydro-11-oxa- 2-aza-1(4,8)-pyrido[2,3-d]pyrimidina-8(4,1)- piperidina-4(1,3)-benzenacyclotridecaphan-1⁷-one | 648.6 |
| A158 | 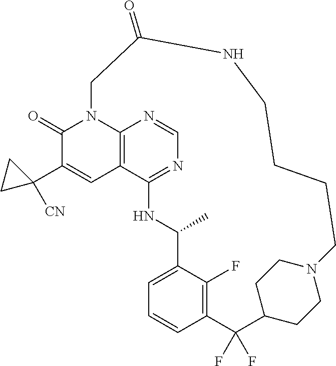 [SMILES: C[C@H]1Nc2ncnc3c2cc(C2(C#N)CC2)c(=O)n3CC(=O)NCCCCN2CCC(CC2)C(F)(F)c2cccc1c2F] | 1-((4R)-3²,2,2-trifluoro-4-methyl-6⁷,8-dioxo-6⁷,6⁸- dihydro-5,9-diaza-6(4,8)-pyrido[2,3-d]pyrimidina- 1(4,1)-piperidina-3(1,3)- benzenacyclotridecaphane-6⁶-yl)cyclopropane-1- carbonitrile | 594.7 |
| A159 | 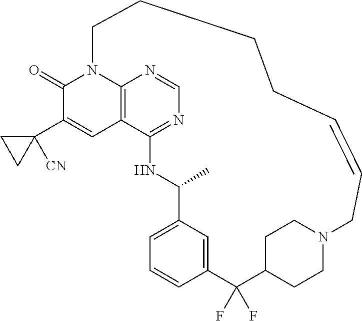 [SMILES: C[C@H]1Nc2ncnc3c2cc(C2(C#N)CC2)c(=O)n3CCCC/C=C\CN2CCC(CC2)C(F)(F)c2cccc1c2] | 1-((4R,Z)-2,2-difluoro-4-methyl-6⁷-oxo-6⁷,6⁸- dihydro-5-aza-6(4,8)-pyrido[2,3-d]pyrimidina- 1(4,1)-piperidina-3(1,3)-benzenacyclotridecaphan- 11-en-6⁶-yl)cyclopropane-1-carbonitrile | 559.4 |
| A160 | 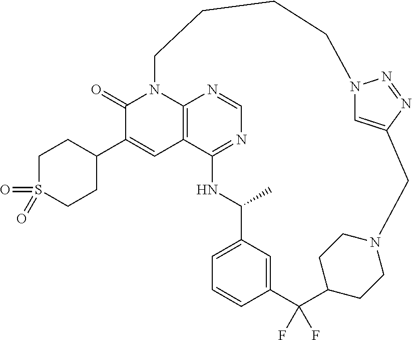 [SMILES: C[C@H]1Nc2ncnc3c2cc(C2CCS(=O)(=O)CC2)c(=O)n3CCCCn2cc(nn2)CN2CCC(CC2)C(F)(F)c2cccc1c2] | (6R,Z)-8⁶-(1,1-dioxidotetrahydro-2H-thiopyran-4- yl)-4,4-difluoro-6-methyl-8⁷,8⁸-dihydro-1¹H-7-aza- 8(4,8)-pyrido[2,3-d]pyrimidina-3(1,4)-piperidina- 1(4,1)-triazola-5(1,3)-benzenacyclododecaphan-8⁷- one | 667.3 |
| A161 | 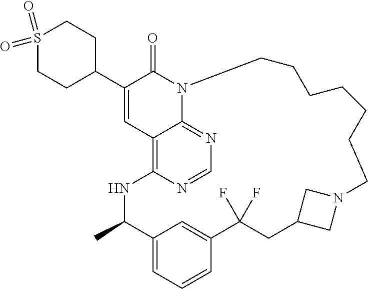 [SMILES: C[C@H]1Nc2ncnc3c2cc(C2CCS(=O)(=O)CC2)c(=O)n3CCCCCCCN2CC(C2)CC(F)(F)c2cccc1c2] | (3R)-1⁶-(1,1-dioxidotetrahydro-2H-thiopyran-4- yl)-5,5-difluoro-3-methyl-1⁷,1⁸-dihydro-2-aza- 1(4,8)-pyrido[2,3-d]pyrimidina-7(3,1)-azetidina- 4(1,3)-benzenacyclotetradecaphan-1⁷-one | 614.3 |
| A162 | 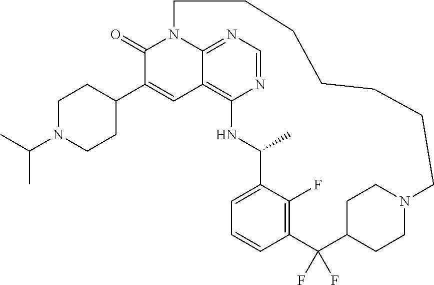 [SMILES: CC(C)N1CCC(c2cc3c4ncnc3n(c2=O)CCCCCCCN2CCC(CC2)C(F)(F)c2cccc(c2F)[C@@H](C)N4)CC1] | (4R)-3²,2,2-trifluoro-6⁶-(1-isopropylpiperidin-4- yl)-4-methyl-6⁷,6⁸-dihydro-5-aza-6(4,8)- pyrido[2,3-d]pyrimidina-1(4,1)-piperidina-3(1,3)- benzenacyclotridecaphan-6⁷-one | 639.7 |
| A163 | 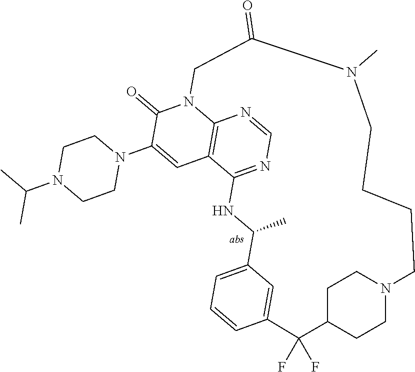 [SMILES: CC(C)N1CCN(c2cc3c4ncnc3n(c2=O)CC(=O)N(C)CCCCN2CCC(CC2)C(F)(F)c2cccc(c2)[C@@H](C)N4)CC1] | (4R)-2,2-difluoro-6⁶-(4-isopropylpiperazin-1-yl)- 4,9-dimethyl-6⁷,6⁸-dihydro-5,9-diaza-6(4,8)- pyrido[2,3-d]pyrimidina-1(4,1)-piperidina-3(1,3)- benzenacyclotridecaphane-6⁷,8-dione | 651.3 |
| A164 | 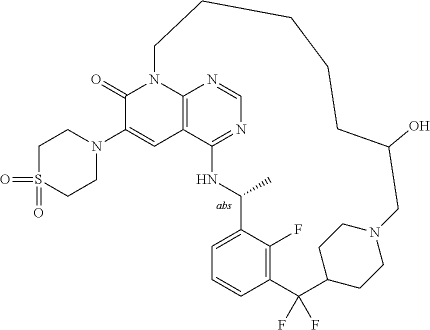 [SMILES: C[C@H]1Nc2ncnc3c2cc(N2CCS(=O)(=O)CC2)c(=O)n3CCCCCC(O)CN2CCC(CC2)C(F)(F)c2cccc1c2F] | (4R)-6⁶-(1,1-dioxidotetrahydro-2H-thiopyran-4- yl)-3²,2,2-trifluoro-12-hydroxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 662.4 |
| A165 | 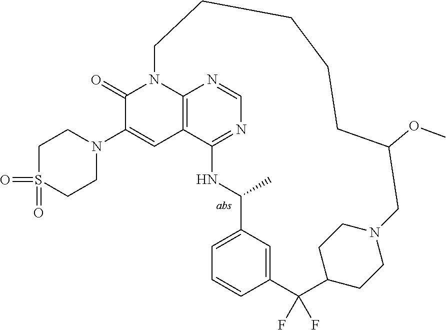 [SMILES: COC1CCCCCn2c(=O)c(N3CCS(=O)(=O)CC3)cc3c(ncnc32)N[C@H](C)c2cccc(c2)C(F)(F)C2CCN(CC2)C1] | (4R)-6⁶-(1,1-dioxidotetrahydro-2H-thiopyran-4- yl)-2,2-difluoro-12-methoxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 658.3 |
| A166 | 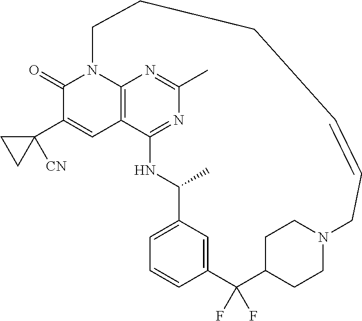 [SMILES: Cc1nc2c3cc(C4(C#N)CC4)c(=O)n(c3n1)CCC/C=C\CN1CCC(CC1)C(F)(F)c1cccc(c1)[C@@H](C)N2] | 1-((4R,Z)-2,2-difluoro-6²,4-dimethyl-6⁷-oxo-6⁷,6⁸- dihydro-5-aza-6(4,8)-pyrido[2,3-d]pyrimidina- 1(4,1)-piperidina-3(1,3)-benzenacyclododecaphan- 10-en-6⁶-yl)cyclopropane-1-carbonitrile | 559.5 |
| A167 | 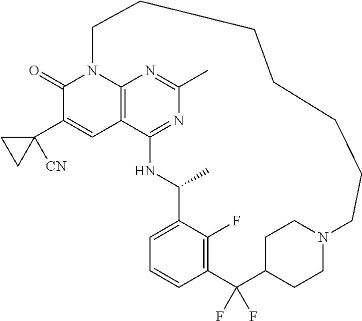 [SMILES: Cc1nc2c3cc(C4(C#N)CC4)c(=O)n(c3n1)CCCCCCCN1CCC(CC1)C(F)(F)c1cccc(c1F)[C@@H](C)N2] | 1-((4R)-3²,2,2-trifluoro-6²,4-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)cyclopropane-1- carbonitrile | 593.5 |
| A168 | 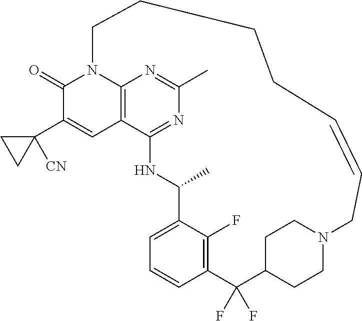 [SMILES: Cc1nc2c3cc(C4(C#N)CC4)c(=O)n(c3n1)CCCC/C=C\CN1CCC(CC1)C(F)(F)c1cccc(c1F)[C@@H](C)N2] | 1-((4R,Z)-3²,2,2-trifluoro-6²,4-dimethyl-6⁷-oxo- 6⁷,6⁸-dihydro-5-aza-6(4,8)-pyrido[2,3- d]pyrimidina-1(4,1)-piperidina-3(1,3)- benzenacyclotridecaphan-11-en-6⁶- yl)cyclopropane-1-carbonitrile | 591.5 |
| A169 | 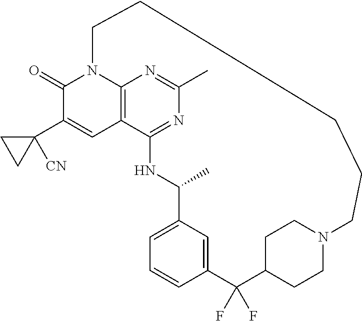 [SMILES: Cc1nc2c3cc(C4(C#N)CC4)c(=O)n(c3n1)CCCCCN1CCC(CC1)C(F)(F)c1cccc(c1)[C@@H](C)N2] | 1-((4R)-2,2-difluoro-6²,4-dimethyl-6⁷-oxo-6⁷,6⁸- dihydro-5-aza-6(4,8)-pyrido[2,3-d]pyrimidina- 1(4,1)-piperidina-3(1,3)- benzenacycloundecaphane-6⁶-yl)cyclopropane-1- carbonitrile | 547.4 |
| A170 | 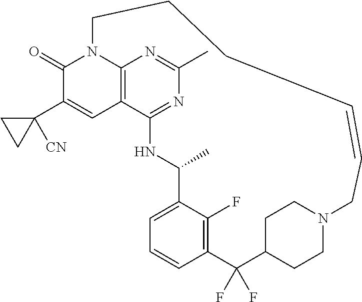 [SMILES: Cc1nc2c3cc(C4(C#N)CC4)c(=O)n(c3n1)CCC/C=C\CN1CCC(CC1)C(F)(F)c1cccc(c1F)[C@@H](C)N2] | 1-((4R,Z)-3²,2,2-trifluoro-6²,4-dimethyl-6⁷-oxo- 6⁷,6⁸-dihydro-5-aza-6(4,8)-pyrido[2,3- d]pyrimidina-1(4,1)-piperidina-3(1,3)- benzenacyclododecaphan-10-en-6⁶- yl)cyclopropane-1-carbonitrile | 577.5 |
| A171 | 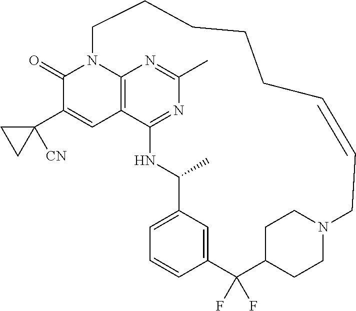 [SMILES: Cc1nc2c3cc(C4(C#N)CC4)c(=O)n(c3n1)CCCCC/C=C\CN1CCC(CC1)C(F)(F)c1cccc(c1)[C@@H](C)N2] | 1-((4R,Z)-2,2-difluoro-6²,4-dimethyl-6⁷-oxo-6⁷,6⁸- dihydro-5-aza-6(4,8)-pyrido[2,3-d]pyrimidina- 1(4,1)-piperidina-3(1,3)- benzenacyclotetradecaphan-12-en-6⁶- yl)cyclopropane-1-carbonitrile | 587.5 |
| A172 | 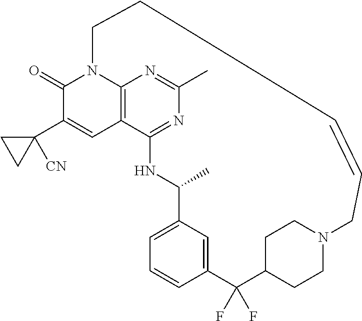 [SMILES: Cc1nc2c3cc(C4(C#N)CC4)c(=O)n(c3n1)CC/C=C\CN1CCC(CC1)C(F)(F)c1cccc(c1)[C@@H](C)N2] | 1-((4R,Z)-2,2-difluoro-6²,4-dimethyl-6⁷-oxo-6⁷,6⁸- dihydro-5-aza-6(4,8)-pyrido[2,3-d]pyrimidina- 1(4,1)-piperidina-3(1,3)-benzenacycloundecaphan- 9-en-6⁶-yl)cyclopropane-1-carbonitrile | 545.4 |
| A173 | 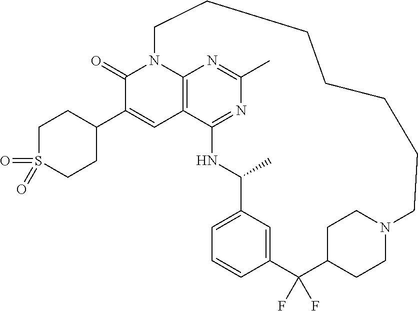 [SMILES: Cc1nc2c3cc(C4CCS(=O)(=O)CC4)c(=O)n(c3n1)CCCCCCCN1CCC(CC1)C(F)(F)c1cccc(c1)[C@@H](C)N2] | (4R)-6⁶-(1,1-dioxidotetrahydro-2H-thiopyran-4- yl)-2,2-difluoro-6²,4-dimethyl-6⁷,6⁸-dihydro-5-aza- 6(4,8)-pyrido[2,3-d]pyrimidina-1(4,1)-piperidina- 3(1,3)-benzenacyclotridecaphan-6⁷-one | 642.5 |
| A174 | 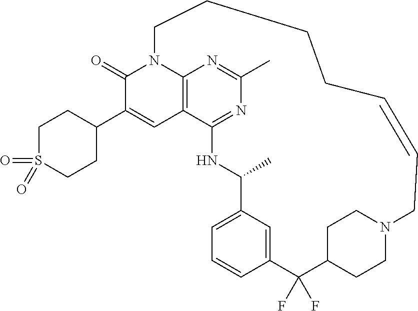 [SMILES: Cc1nc2c3cc(C4CCS(=O)(=O)CC4)c(=O)n(c3n1)CCCC/C=C\CN1CCC(CC1)C(F)(F)c1cccc(c1)[C@@H](C)N2] | (4R,Z)-6⁶-(1,1-dioxidotetrahydro-2H-thiopyran-4- yl)-2,2-difluoro-6²,4-dimethyl-6⁷,6⁸-dihydro-5-aza- 6(4,8)-pyrido[2,3-d]pyrimidina-1(4,1)-piperidina- 3(1,3)-benzenacyclotridecaphan-11-en-6⁷-one | 640.5 |
| A175 | 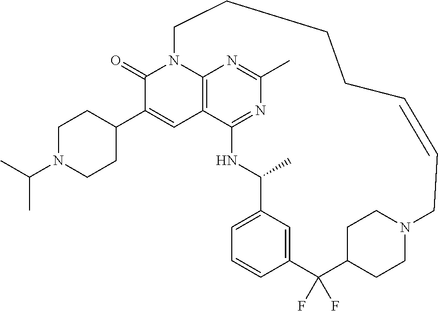 [SMILES: Cc1nc2c3cc(C4CCN(C(C)C)CC4)c(=O)n(c3n1)CCCC/C=C\CN1CCC(CC1)C(F)(F)c1cccc(c1)[C@@H](C)N2] | (4R,Z)-2,2-difluoro-6⁶-(1-isopropylpiperidin-4-yl)- 6²,4-dimethyl-6⁷,6⁸-dihydro-5-aza-6(4,8)- pyrido[2,3-d]pyrimidina-1(4,1)-piperidina-3(1,3)- benzenacyclotridecaphan-11-en-6⁷-one | 633.5 |
| A176 | 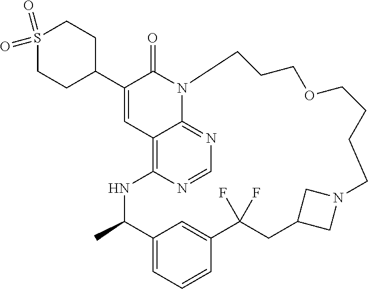 [SMILES: C[C@H]1Nc2ncnc3c2cc(C2CCS(=O)(=O)CC2)c(=O)n3CCCOCCCCN2CC(C2)CC(F)(F)c2cccc1c2] | (3R)-1⁶-(1,1-dioxidotetrahydro-2H-thiopyran-4- yl)-5,5-difluoro-3-methyl-1⁷,1⁸-dihydro-12-oxa-2- aza-1(4,8)-pyrido[2,3-d]pyrimidina-7(3,1)- azetidina-4(1,3)-benzenacyclopentadecaphan-1⁷- one | 630.4 |
| A177 | 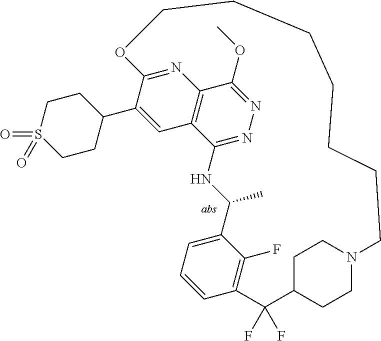 [SMILES: COc1nnc2c3cc(C4CCS(=O)(=O)CC4)c(nc13)OCCCCCCCN1CCC(CC1)C(F)(F)c1cccc(c1F)[C@@H](C)N2] | (R)-4-(3²,2,2-trifluoro-68-methoxy-4-methyl-7- oxa-5-aza-6(5,2)-pyrido[2,3-d]pyridazina-1(4,1)- piperidina-3(1,3)-benzenacyclotetradecaphane-6³- yl)tetrahydro-2H-thiopyran 1,1-dioxide | 676.3 |
| A178 | 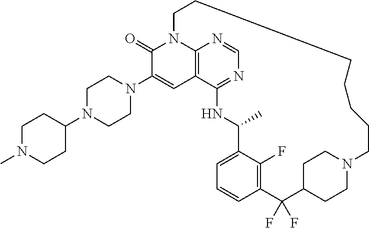 [SMILES: C[C@H]1Nc2ncnc3c2cc(N2CCN(C4CCN(C)CC4)CC2)c(=O)n3CCCCCCCN2CCC(CC2)C(F)(F)c2cccc1c2F] | (4R)-3²,2,2-trifluoro-4-methyl-6⁶-(4-(1- methylpiperidin-4-yl)piperazin-1-yl)-6⁷,6⁸- dihydro-5-aza-6(4,8)-pyrido[2,3-d]pyrimidina- 1(4,1)-piperidina-3(1,3)- benzenacyclotridecaphan-6⁷-one | 695.5 |
| A179 | 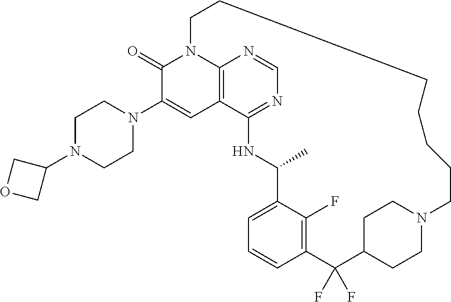 [SMILES: C[C@H]1Nc2ncnc3c2cc(N2CCN(C4COC4)CC2)c(=O)n3CCCCCCCN2CCC(CC2)C(F)(F)c2cccc1c2F] | (4R)-3²,2,2-trifluoro-4-methyl-6⁶-(4-(oxetan-3- yl)piperazin-1-yl)-6⁷,6⁸-dihydro-5-aza-6(4,8)- pyrido[2,3-d]pyrimidina-1(4,1)-piperidina-3(1,3)- benzenacyclotridecaphan-6⁷-one | 654.3 |
| A180 | 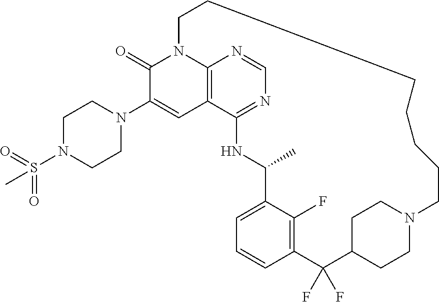 [SMILES: C[C@H]1Nc2ncnc3c2cc(N2CCN(S(C)(=O)=O)CC2)c(=O)n3CCCCCCCN2CCC(CC2)C(F)(F)c2cccc1c2F] | (4R)-3²,2,2-trifluoro-4-methyl-6⁶-(4- (methylsulfonyl)piperazin-1-yl)-6⁷,6⁸-dihydro-5- aza-6(4,8)-pyrido[2,3-d]pyrimidina-1(4,1)- piperidina-3(1,3)- benzenacyclotridecaphan-6⁷-one | 676.4 |
| A181 | 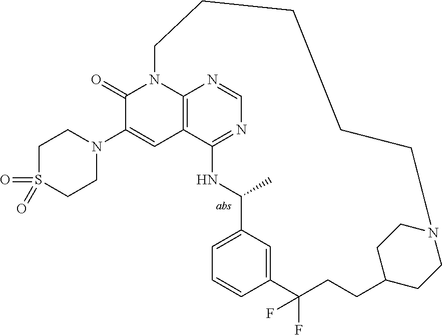 [SMILES: C[C@H]1Nc2ncnc3c2cc(N2CCS(=O)(=O)CC2)c(=O)n3CCCCCN2CCC(CC2)CCC(F)(F)c2cccc1c2] | (3R)-1⁶-(1,1-dioxidothiomorpholino)-5,5-difluoro- 3-methyl-1⁷,1⁸-dihydro-2-aza-1(4,8)-pyrido[2,3- d]pyrimidina-8(4,1)-piperidina-4(1,3)- benzenacyclotridecaphan-1⁷-one | 629.3 |
| A182 | 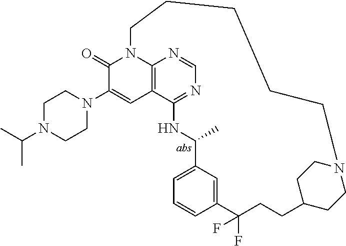 [SMILES: CC(C)N1CCN(c2cc3c4ncnc3n(c2=O)CCCCCN2CCC(CC2)CCC(F)(F)c2cccc(c2)[C@@H](C)N4)CC1] | (3R)-5,5-difluoro-1⁶-(1-isopropylpiperidin-4-yl)-3- methyl-1⁷,1⁸-dihydro-2-aza-1(4,8)-pyrido[2,3- d]pyrimidina-8(4,1)-piperidina-4(1,3)- benzenacyclotridecaphan-1⁷-one | 621.4 |
| A183 | 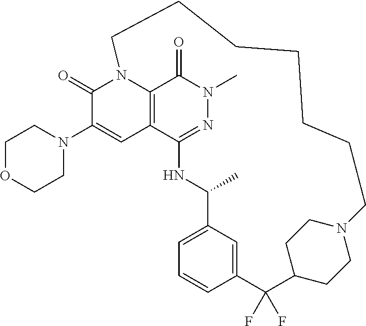 [SMILES: C[C@H]1Nc2nn(C)c(=O)c3c2cc(N2CCOCC2)c(=O)n3CCCCCCCN2CCC(CC2)C(F)(F)c2cccc1c2] | (4R,E)-2,2-difluoro-6⁷,4-dimethyl-6³-morpholino- 6¹,6²,6⁷,6⁸-tetrahydro-5-aza-6(5,1)-pyrido[2,3- d]pyridazina-1(4,1)-piperidina-3(1,3)- benzenacyclotridecaphane-6²,6⁸-dione | 611.5 |
| A184 | 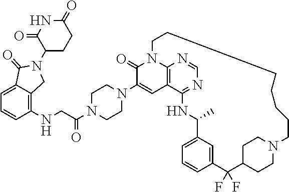 [SMILES: C[C@H]1Nc2ncnc3c2cc(N2CCN(C(=O)CNc4cccc5c4CN(C4CCC(=O)NC4=O)C5=O)CC2)c(=O)n3CCCCCCCN2CCC(CC2)C(F)(F)c2cccc1c2] | 3-(4-((2-(4-((4R)-2,2-difluoro-4-methy1-6⁷-oxo- 6⁷,6⁸-dihydro-5-aza-6(4,8)-pyrido[2,3- d]pyrimidina-1(4,1)-piperidina-3(1,3)- benzenacyclotridecaphane-6⁶-yl)piperazin-1-yl)-2- oxoethyl)amino)-1-oxoisoindolin-2-yl)piperidine- 2,6-dione | 879.7 |
| A185 | 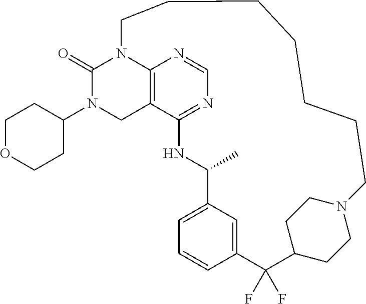 [SMILES: C[C@H]1Nc2ncnc3c2CN(C2CCOCC2)C(=O)N3CCCCCCCN2CCC(CC2)C(F)(F)c2cccc1c2] | (4R)-2,2-difluoro-4-methyl-6³-(tetrahydro-2H- pyran-4-yl)-6¹,6²,6³,6⁴-tetrahydro-5-aza-6(5,1)- pyrimido[4,5-d]pyrimidina-1(4,1)-piperidina- 3(1,3)-benzenacyclotridecaphan-6²-one | 583.5 |
| A186 | 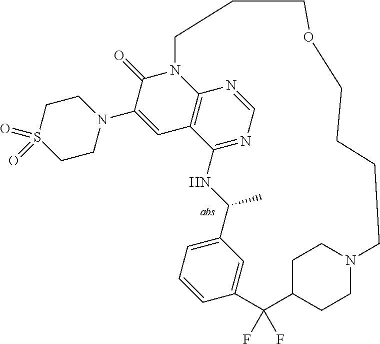 [SMILES: C[C@H]1Nc2ncnc3c2cc(N2CCS(=O)(=O)CC2)c(=O)n3CCCOCCCCN2CCC(CC2)C(F)(F)c2cccc1c2] | (4R)-6⁶-(1,1-dioxidothiomorpholino)-2,2-difluoro- 4-methyl-6⁷,6⁸-dihydro-10-oxa-5-aza-6(4,8)- pyrido[2,3-d]pyrimidina-1(4,1)-piperidina-3(1,3)- benzenacyclotetradecaphan-6⁷-one | 645.3 |
| A187 | 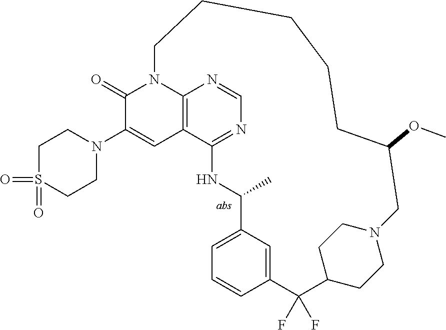 [SMILES: CO[C@@H]1CCCCCn2c(=O)c(N3CCS(=O)(=O)CC3)cc3c(ncnc32)N[C@H](C)c2cccc(c2)C(F)(F)C2CCN(CC2)C1] | (4R,12R)-6⁶-(1,1-dioxidotetrahydro-2H-thiopyran- 4-yl)-2,2-difluoro-12-methoxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 658.3 |
| A188 | 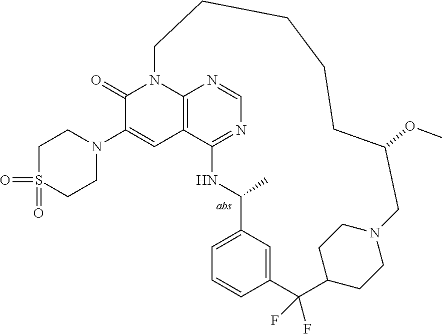 [SMILES: CO[C@H]1CCCCCn2c(=O)c(N3CCS(=O)(=O)CC3)cc3c(ncnc32)N[C@H](C)c2cccc(c2)C(F)(F)C2CCN(CC2)C1] | (4R,12S)-6⁶-(1,1-dioxidotetrahydro-2H-thiopyran- 4-yl)-2,2-difluoro-12-methoxy-4-methyl-6⁷,6⁸- dihydro-5-aza-6(4,8)-pyrido[2,3-d]pyrimidina- 1(4,1)-piperidina-3(1,3)-benzenacyclotridecaphan- 6⁷-one | 658.6 |
| A189 | 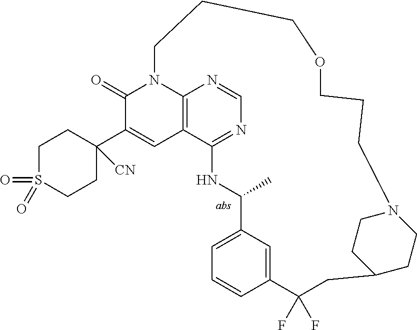 [SMILES: C[C@H]1Nc2ncnc3c2cc(C2(C#N)CCS(=O)(=O)CC2)c(=O)n3CCCOCCCN2CCC(CC2)CC(F)(F)c2cccc1c2] | 4-((3R)-5,5-difluoro-3-methyl-1⁷-oxo-1⁷,1⁸- dihydro-11-oxa-2-aza-1(4,8)-pyrido[2,3- d]pyrimidina-7(4,1)-piperidina-4(1,3)- benzenacyclotetradecaphane-1⁶-yl)tetrahydro-2H- thiopyran-4-carbonitrile 1,1-dioxide | 669.3 |
| A190 | 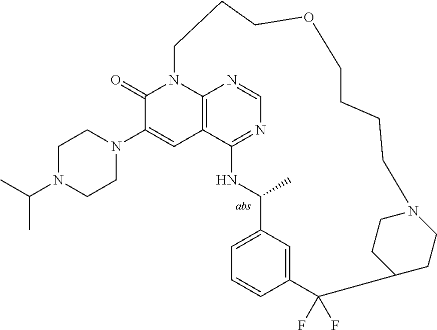 [SMILES: CC(C)N1CCN(c2cc3c4ncnc3n(c2=O)CCCOCCCCN2CCC(CC2)C(F)(F)c2cccc(c2)[C@@H](C)N4)CC1] | (4R)-2,2-difluoro-6⁶-(4-isopropylpiperazin-1-yl)-4- methyl-6⁷,6⁸-dihydro-10-oxa-5-aza-6(4,8)- pyrido[2,3-d]pyrimidina-1(4,1)-piperidina-3(1,3)- benzenacyclotetradecaphan-6⁷-one | 638.4 |
| A191 | 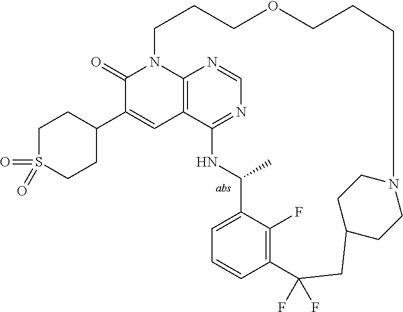 [SMILES: C[C@H]1Nc2ncnc3c2cc(C2CCS(=O)(=O)CC2)c(=O)n3CCCOCCCN2CCC(CC2)CC(F)(F)c2cccc1c2F] | (3R)-1⁶-(1,1-dioxidotetrahydro-2H-thiopyran-4- yl)-4²,5,5-trifluoro-3-methyl-1⁷,1⁸-dihydro-11-oxa- 2-aza-1(4,8)-pyrido[2,3-d]pyrimidina-7(4,1)- piperidina-4(1,3)-benzenacyclotetradecaphan-1⁷- one | 662.3 |
| A192 | 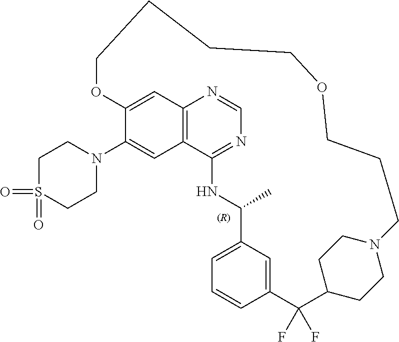 | (R)-4-(2,2-difluoro-4-methyl-7,12-dioxa-5-aza- 6(4,7)-quinazolina-1(4,1)-piperidina-3(1,3)- benzenacyclopentadecaphane-6⁶-yl)thiomorpholine 1,1-dioxide | 644.4 |
| A193 | 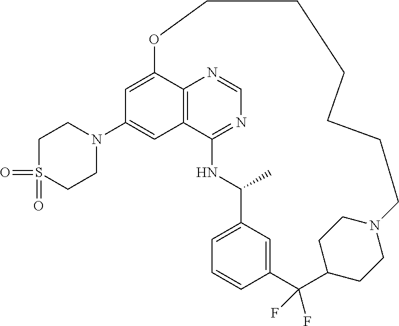 [SMILES: C[C@H]1Nc2ncnc3c(cc(N4CCS(=O)(=O)CC4)cc23)OCCCCCCN2CCC(CC2)C(F)(F)c2cccc1c2] | (R)-4-(2,2-difluoro-4-methyl-7-oxa-5-aza-6(4,8)- quinazolina-1(4,1)-piperidina-3(1,3)- benzenacyclotridecaphane-6⁶-yl)thiomorpholine 1,1-dioxide | 614.6 |
| A194 | 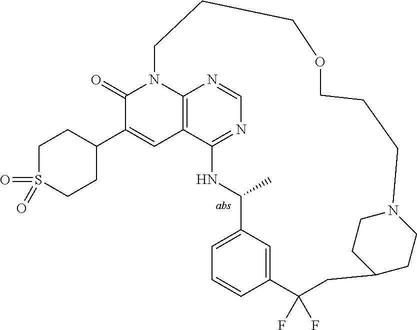 [SMILES: C[C@H]1Nc2ncnc3c2cc(C2CCS(=O)(=O)CC2)c(=O)n3CCCOCCCN2CCC(CC2)CC(F)(F)c2cccc1c2] | (3R)-1⁶-(1,1-dioxidotetrahydro-2H-thiopyran-4- yl)-5,5-difluoro-3-methyl-1⁷,1⁸-dihydro-11-oxa-2- aza-1(4,8)-pyrido[2,3-d]pyrimidina-7(4,1)- piperidina-4(1,3)-benzenacyclotetradecaphan-1⁷- one | 644.3 |
| A195 | 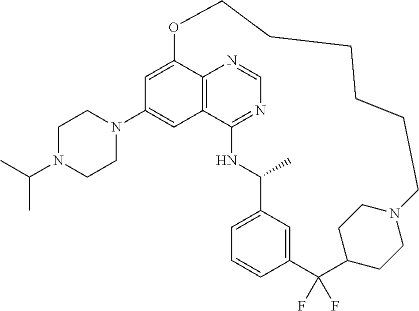 [SMILES: CC(C)N1CCN(c2cc3c4ncnc(c4c2)N[C@H](C)c2cccc(c2)C(F)(F)C2CCN(CCCCCCO3)CC2)CC1] | (R)-2,2-difluoro-6⁶-(4-isopropylpiperazin-1-yl)-4- methyl-7-oxa-5-aza-6(4,8)-quinazolina-1(4,1)- piperidina-3(1,3)-benzenacyclotridecaphane | 607.5 |
| A196 | 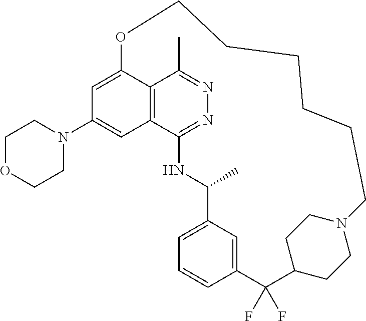 [SMILES: Cc1nnc2c3cc(N4CCOCC4)cc(c13)OCCCCCCN1CCC(CC1)C(F)(F)c1cccc(c1)[C@@H](C)N2] | (R)-2,2-difluoro-6⁴,4-dimethyl-6⁷-morpholino-7- oxa-5-aza-6(1,5)-phthalazina-1(4,1)-piperidina- 3(1,3)-benzenacyclotridecaphane | 580.5 |
| A197 | 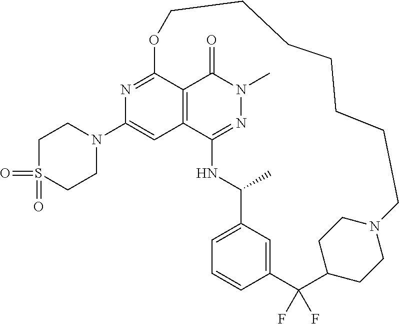 [SMILES: C[C@H]1Nc2nn(C)c(=O)c3c(nc(N4CCS(=O)(=O)CC4)cc23)OCCCCCCCN2CCC(CC2)C(F)(F)c2cccc1c2] | (R)-6⁷-(1,1-dioxidothiomorpholino)-2,2-difluoro- 6³,4-dimethyl-6³,6⁴-dihydro-7-oxa-5-aza-6(1,5)- pyrido[3,4-d]pyridazina-1(4,1)-piperidina-3(1,3)- benzenacyclotetradecaphan-64-one | 659.3 |
| A198 | 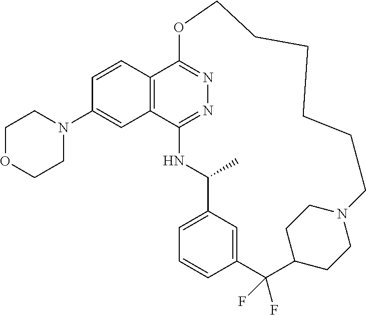 [SMILES: C[C@H]1Nc2nnc(c3ccc(N4CCOCC4)cc23)OCCCCCCN2CCC(CC2)C(F)(F)c2cccc1c2] | (R)-2,2-difluoro-4-methyl-6⁷-morpholino-7-oxa-5- aza-6(1,4)-phthalazina-1(4,1)-piperidina-3(1,3)- benzenacyclotridecaphane | |
| A200 | 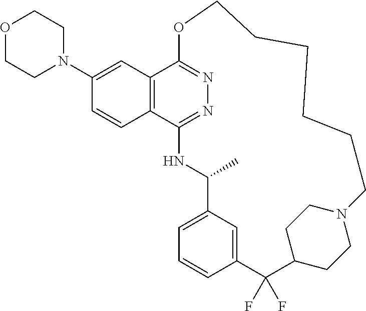 [SMILES: C[C@H]1Nc2nnc(c3cc(N4CCOCC4)ccc23)OCCCCCCN2CCC(CC2)C(F)(F)c2cccc1c2] | (R)-2,2-difluoro-4-methyl-6⁶-morpholino-7-oxa-5- aza-6(1,4)-phthalazina-1(4,1)-piperidina-3(1,3)- benzenacyclotridecaphane | |
| A201 | 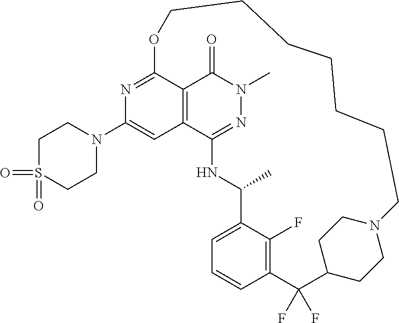 [SMILES: C[C@H]1Nc2nn(C)c(=O)c3c(nc(N4CCS(=O)(=O)CC4)cc23)OCCCCCCCN2CCC(CC2)C(F)(F)c2cccc1c2F] | (R)-6⁷-(1,1-dioxidothiomorpholino)-3²,2,2- trifluoro-6³,4-dimethyl-6³,6⁴-dihydro-7-oxa-5-aza- 6(1,5)-pyrido[3,4-d]pyridazina-1(4,1)-piperidina- 3(1,3)-benzenacyclotetradecaphan-6⁴-one | |
| A202 | 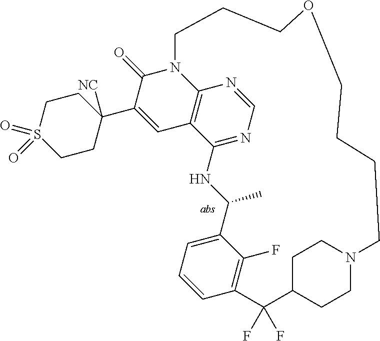 [SMILES: C[C@H]1Nc2ncnc3c2cc(C2(C#N)CCS(=O)(=O)CC2)c(=O)n3CCCOCCCCN2CCC(CC2)C(F)(F)c2cccc1c2F] | 4-((4R)-3²,2,2-trifluoro-4-methyl-6⁷-oxo-6⁷,6⁸- dihydro-10-oxa-5-aza-6(4,8)-pyrido[2,3- d]pyrimidina-1(4,1)-piperidina-3(1,3)- benzenacyclotetradecaphane-6⁶-yl)tetrahydro-2H- thiopyran-4-carbonitrile 1,1-dioxide | 687.3 |
| A203 | 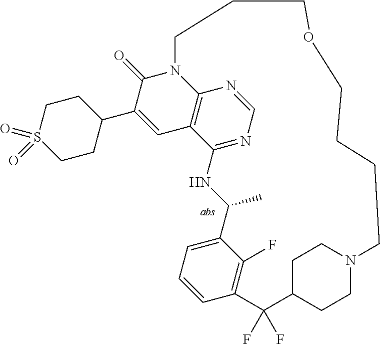 [SMILES: C[C@H]1Nc2ncnc3c2cc(C2CCS(=O)(=O)CC2)c(=O)n3CCCOCCCCN2CCC(CC2)C(F)(F)c2cccc1c2F] | (4R)-6⁶-(1,1-dioxidotetrahydro-2H-thiopyran-4- yl)-3²,2,2-trifluoro-4-methyl-6⁷,6⁸-dihydro-10-oxa- 5-aza-6(4,8)-pyrido[2,3-d]pyrimidina-1(4,1)- piperidina-3(1,3)-benzenacyclotetradecaphan-6⁷- one | 662.3 |
| A204 | 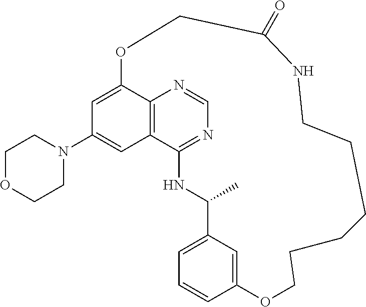 [SMILES: C[C@H]1Nc2ncnc3c(cc(N4CCOCC4)cc23)OCC(=O)NCCCCCCOc2cccc1c2] | (R)-3-methyl-1⁶-morpholino-5,15-dioxa-2,12- diaza-1(4,8)-quinazolina-4(1,3)- benzenacyclopentadecaphan-13-one | 506.4 |
| A205 | 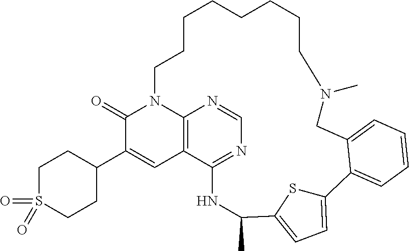 [SMILES: C[C@H]1Nc2ncnc3c2cc(C2CCS(=O)(=O)CC2)c(=O)n3CCCCCCCCN(C)Cc2ccccc2C2=CC=C1S2] | (3R)-5⁶-(1,1-dioxidotetrahydro-2H-thiopyran-4- yl)-3,14-dimethyl-5⁷,5⁸-dihydro-4,14-diaza-5(4,8)- pyrido[2,3-d]pyrimidina-2(2,5)-thiophena-1(1,2)- benzenacyclopentadecaphan-5⁷-one | 634.5 |
| A206 | 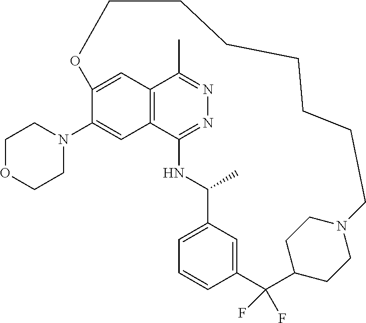 [SMILES: Cc1nnc2c3cc(N4CCOCC4)c(cc13)OCCCCCCCN1CCC(CC1)C(F)(F)c1cccc(c1)[C@@H](C)N2] | (R)-2,2-difluoro-6⁴,4-dimethyl-6⁷-morpholino-7- oxa-5-aza-6(1,6)-phthalazina-1(4,1)-piperidina- 3(1,3)-benzenacyclotetradecaphane | 594.5 |
| A207 | 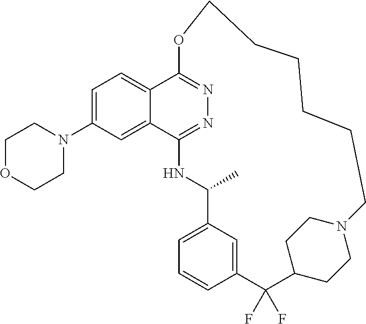 [SMILES: C[C@H]1Nc2nnc(c3ccc(N4CCOCC4)cc23)OCCCCCCN2CCC(CC2)C(F)(F)c2cccc1c2] | (R)-2,2-difluoro-4-methyl-6⁷-morpholino-7-oxa-5- aza-6(1,4)-phthalazina-1(4,1)-piperidina-3(1,3)- benzenacyclotridecaphane and (R)-2,2-difluoro-4-methyl-6⁶-morpholino-7-oxa-5- aza-6(1,4)-phthalazina-1(4,1)-piperidina-3(1,3)- benzenacyclotridecaphane (mixture of regioisomers) | 566.2 |
| A208 | 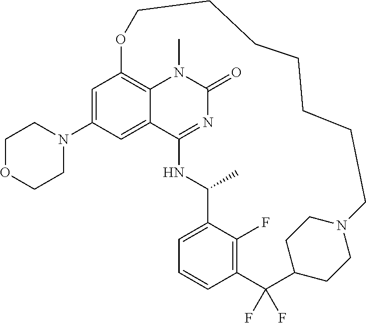 [SMILES: C[C@H]1Nc2nc(=O)n(C)c3c(cc(N4CCOCC4)cc23)OCCCCCCCN2CCC(CC2)C(F)(F)c2cccc1c2F] | (R)-3²,2,2-trifluoro-6¹,4-dimethyl-6⁶-morpholino- 6¹,6²-dihydro-7-oxa-5-aza-6(4,8)-quinazolina- 1(4,1)-piperidina-3(1,3)- benzenacyclotetradecaphan-6²-one | 628.4 |
| A209 | 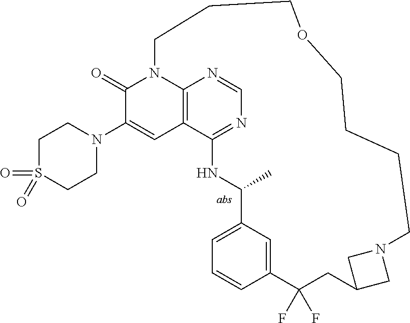 [SMILES: C[C@H]1Nc2ncnc3c2cc(N2CCS(=O)(=O)CC2)c(=O)n3CCCOCCCCN2CC(C2)CC(F)(F)c2cccc1c2] | (3R)-1⁶-(1,1-dioxidothiomorpholino)-5,5-difluoro- 3-methyl-1⁷,1⁸-dihydro-12-oxa-2-aza-1(4,8)- pyrido[2,3-d]pyrimidina-7(3,1)-azetidina-4(1,3)- benzenacyclopentadecaphan-1⁷-one | 631.6 |
| A210 | 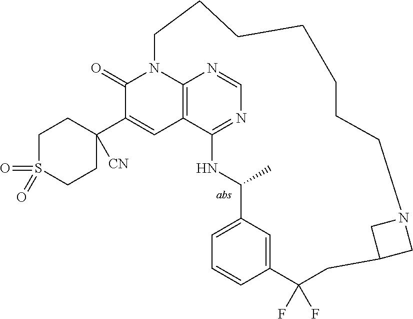 [SMILES: C[C@H]1Nc2ncnc3c2cc(C2(C#N)CCS(=O)(=O)CC2)c(=O)n3CCCCCCCN2CC(C2)CC(F)(F)c2cccc1c2] | 4-((3R)-5,5-difluoro-3-methyl-1⁷-oxo-1⁷,1⁸- dihydro-2-aza-1(4,8)-pyrido[2,3-d]pyrimidina- 7(3,1)-azetidina-4(1,3)- benzenacyclotetradecaphane-1⁶-yl)tetrahydro-2H- thiopyran-4-carbonitrile 1,1-dioxide | 639.5 |
| A211 | 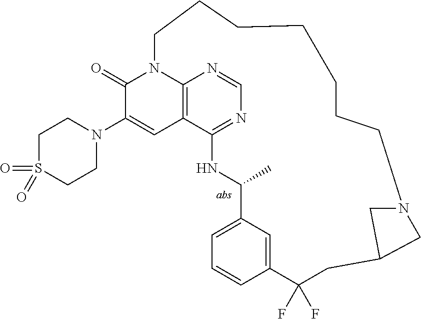 [SMILES: C[C@H]1Nc2ncnc3c2cc(N2CCS(=O)(=O)CC2)c(=O)n3CCCCCCCN2CC(C2)CC(F)(F)c2cccc1c2] | (3R)-1⁶-(1,1-dioxidothiomorpholino)-5,5-difluoro- 3-methyl-1⁷,1⁸-dihydro-2-aza-1(4,8)-pyrido[2,3- d]pyrimidina-7(3,1)-azetidina-4(1,3)- benzenacyclotetradecaphan-1⁷-one | 615.3 |
| A212 | 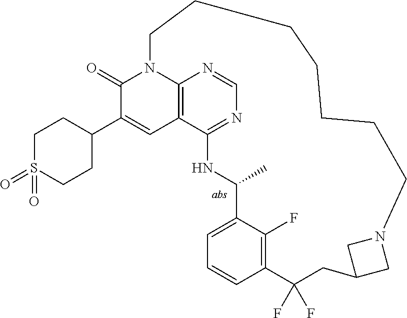 [SMILES: C[C@H]1Nc2ncnc3c2cc(C2CCS(=O)(=O)CC2)c(=O)n3CCCCCCCN2CC(C2)CC(F)(F)c2cccc1c2F] | (3R)-1⁶-(1,1-dioxidotetrahydro-2H-thiopyran-4- yl)-4²,5,5-trifluoro-3-methyl-1⁷,1⁸-dihydro-2-aza- 1(4,8)-pyrido[2,3-d]pyrimidina-7(3,1)-azetidina- 4(1,3)-benzenacyclotetradecaphan-1⁷-one | 632.0 |
| A213 | 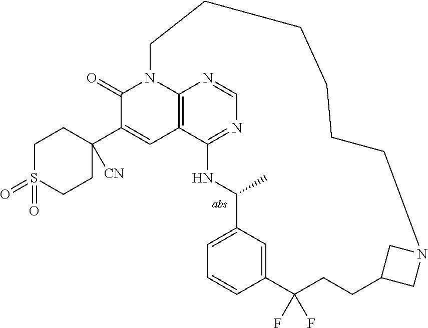 [SMILES: C[C@H]1Nc2ncnc3c2cc(C2(C#N)CCS(=O)(=O)CC2)c(=O)n3CCCCCCN2CC(CCC(F)(F)c3cccc1c3)C2] | 4-((3R)-5,5-difluoro-3-methyl-1⁷-oxo-1⁷,1⁸- dihydro-2-aza-1(4,8)-pyrido[2,3-d]pyrimidina- 8(3,1)-azetidina-4(1,3)- benzenacyclotetradecaphane-1⁶-yl)tetrahydro-2H- thiopyran-4-carbonitrile 1,1-dioxide | 639.2 |

TABLE 2

| | | | [M + |
|---|---|---|---|
| No. | Structure | Chemical Name | H]⁺ |
| B101 | 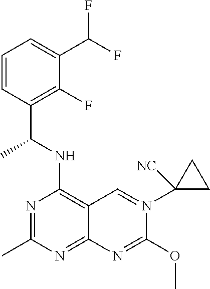 | 1-(4-((R)-1-(2-fluoro-3- (difluoromethyl)phenyl)ethylamino)-7-methoxy- 2-methylpyrido[2,3-d]pyrimidin-6- yl)cyclopropanecarbonitrile | 428.9 |
| B102 | 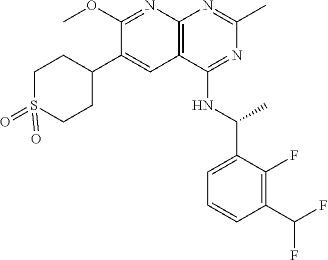 [SMILES: COc1nc2nc(C)nc(N[C@H](C)c3cccc(C(F)F)c3F)c2cc1C1CCS(=O)(=O)CC1] | (R)-4-(4-((1-(3-(difluoromethyl)-2- fluorophenyl)ethyl)amino)-7-methoxy-2- methylpyrido[2,3-d]pyrimidin-6-yl)tetrahydro- 2H-thiopyran 1,1-dioxide | 495 |
| B103 | 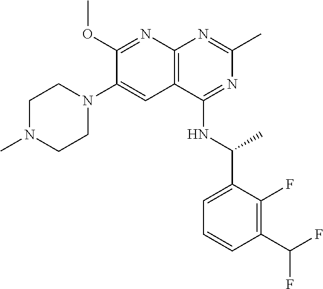 [SMILES: COc1nc2nc(C)nc(N[C@H](C)c3cccc(C(F)F)c3F)c2cc1N1CCN(C)CC1] | (R)-N-(1-(3-(difluoromethyl)-2- fluorophenyl)ethyl)-7-methoxy-2-methyl-6-(4- methylpiperazin-1-yl)pyrido[2,3-d]pyrimidin-4- amine | 461.2 |
| B104 | 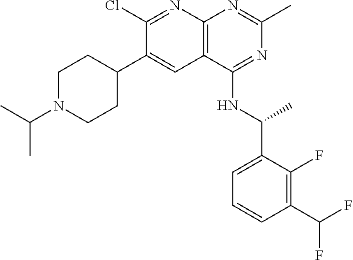 [SMILES: Cc1nc(N[C@H](C)c2cccc(C(F)F)c2F)c2cc(C3CCN(C(C)C)CC3)c(Cl)nc2n1] | (R)-7-chloro-N-(1-(3-(difluoromethyl)-2- fluorophenyl)ethyl)-6-(1-isopropylpiperidin-4- yl)-2-methylpyrido[2,3-d]pyrimidin-4-amine | 492 |
| B105 | 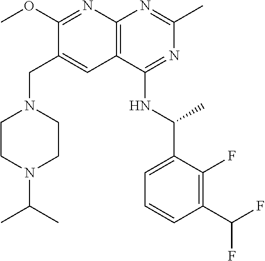 [SMILES: COc1nc2nc(C)nc(N[C@H](C)c3cccc(C(F)F)c3F)c2cc1CN1CCN(C(C)C)CC1] | (R)-N-(1-(3-(difluoromethyl)-2- fluorophenyl)ethyl)-6-((4-isopropylpiperazin-1- yl)methyl)-7-methoxy-2-methylpyrido[2,3- d]pyrimidin-4-amine | 503 |
| B106 | 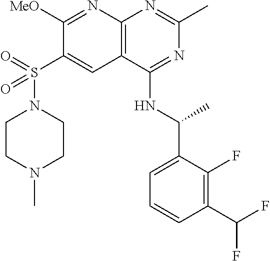 [SMILES: COc1nc2nc(C)nc(N[C@H](C)c3cccc(C(F)F)c3F)c2cc1S(=O)(=O)N1CCN(C)CC1] | (R)-N-(1-(3-(difluoromethyl)-2- fluorophenyl)ethyl)-7-methoxy-2-methyl-6-((4- methylpiperazin-1-yl)sulfonyl)pyrido[2,3- d]pyrimidin-4-amine | 525 |
| B107 | 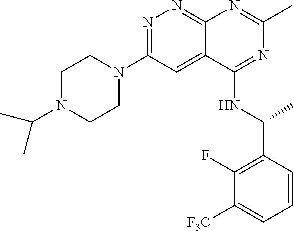 [SMILES: Cc1nc(N[C@H](C)c2cccc(C(F)(F)F)c2F)c2cc(N3CCN(C(C)C)CC3)nnc2n1] | (R)-N-(1-(2-fluoro-3- (trifluoromethyl)phenyl)ethyl)-3-(4- isopropylpiperazin-1-yl)-7-methylpyrimido[4,5- c]pyridazin-5-amine | 478 |
| B108 | 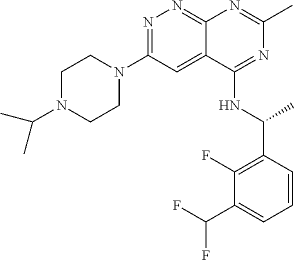 [SMILES: Cc1nc(N[C@H](C)c2cccc(C(F)F)c2F)c2cc(N3CCN(C(C)C)CC3)nnc2n1] | (R)-N-(1-(3-(difluoromethyl)-2- fluorophenyl)ethyl)-3-(4-isopropylpiperazin-1- yl)-7-methylpyrimido[4,5-c]pyridazin-5-amine | 460 |
| B109 | 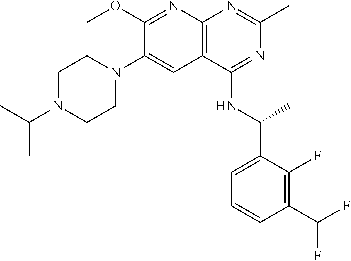 [SMILES: COc1nc2nc(C)nc(N[C@H](C)c3cccc(C(F)F)c3F)c2cc1N1CCN(C(C)C)CC1] | (R)-N-(1-(3-(difluoromethyl)-2-fluoro- phenyl)ethyl)-6-(4-isopropylpiperazin-1-yl)- 7-methoxy-2-methylpyrido[2,3-d]pyrimidin-4- amine | 489.3 |
| B110 | 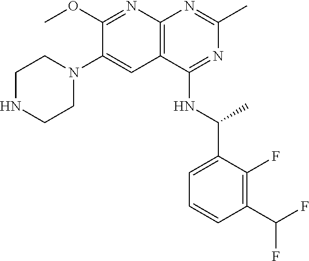 [SMILES: COc1nc2nc(C)nc(N[C@H](C)c3cccc(C(F)F)c3F)c2cc1N1CCNCC1] | (R)-N-(1-(3-(difluoromethyl)-2- fluorophenyl)ethyl)-7-methoxy-2-methyl-6- (piperazin-1-yl)pyrido[2,3-d]pyrimidin-4-amine | 447.4 |
| B111 | 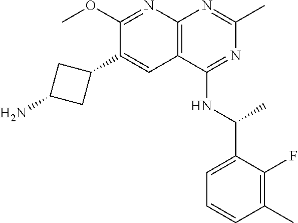 [SMILES: COc1nc2nc(C)nc(N[C@H](C)c3cccc(C)c3F)c2cc1[C@H]1C[C@@H](N)C1] | 6-((1s,3S)-3-aminocyclobutyl)-N-((R)-1-(2- fluoro-3-methylphenyl)ethyl)-7-methoxy-2- methylpyrido[2,3-d]pyrimidin-4-amine | 396.4 |
| B112 | 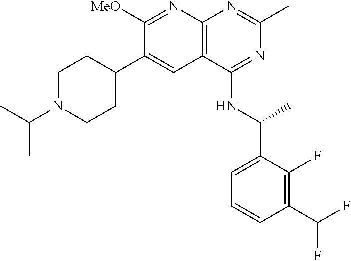 [SMILES: COc1nc2nc(C)nc(N[C@H](C)c3cccc(C(F)F)c3F)c2cc1C1CCN(C(C)C)CC1] | (R)-N-(1-(3-(difluoromethyl)-2-fluoro- phenyl)ethyl)-6-(1-isopropylpiperidin-4-yl)- 7-methoxy-2-methylpyrido[2,3-d]pyrimidin-4- amine | 488.5 |
| B113 | 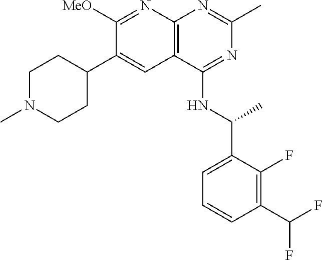 [SMILES: COc1nc2nc(C)nc(N[C@H](C)c3cccc(C(F)F)c3F)c2cc1C1CCN(C)CC1] | (R)-N-(1-(3-(difluoromethyl)-2- fluorophenyl)ethyl)-7-methoxy-2-methyl-6-(1- methylpiperidin-4-yl)pyrido[2,3-d]pyrimidin-4- amine | 460.4 |
| B114 | 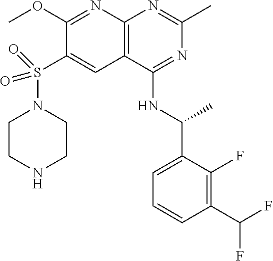 [SMILES: COc1nc2nc(C)nc(N[C@H](C)c3cccc(C(F)F)c3F)c2cc1S(=O)(=O)N1CCNCC1] | (R)-N-(1-(3-(difluoromethyl)-2- fluorophenyl)ethyl)-7-methoxy-2-methyl-6- (piperazin-1-ylsulfonyl)pyrido[2,3-d]pyrimidin-4- amine | 511.4 |
| B115 | 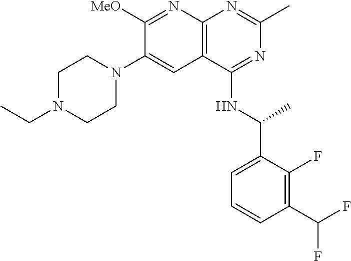 [SMILES: CCN1CCN(c2cc3c(N[C@H](C)c4cccc(C(F)F)c4F)nc(C)nc3nc2OC)CC1] | (R)-N-(1-(3-(difluoromethyl)-2- fluorophenyl)ethyl)-6-(4-ethylpiperazin-1-yl)-7- methoxy-2-methylpyrido[2,3-d]pyrimidin-4- amine | 475.4 |
| B116 | 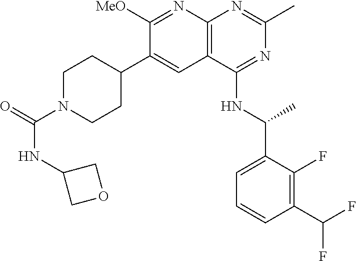 [SMILES: COc1nc2nc(C)nc(N[C@H](C)c3cccc(C(F)F)c3F)c2cc1C1CCN(C(=O)NC2COC2)CC1] | (R)-4-(4-((1-(3-(difluoromethyl)-2- fluorophenyl)ethyl)amino)-7-methoxy-2- methylpyrido[2,3-d]pyrimidin-6-yl)-N-(oxetan- 3-yl)piperidine-1-carboxamide | 546.0 |
| B117 | 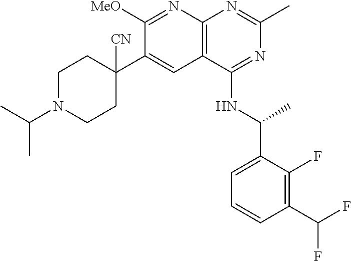 [SMILES: COc1nc2nc(C)nc(N[C@H](C)c3cccc(C(F)F)c3F)c2cc1C1(C#N)CCN(C(C)C)CC1] | (R)-4-(4-((1-(3-(difluoromethyl)-2- fluorophenyl)ethyl)amino)-7-methoxy-2- methylpyrido[2,3-d]pyrimidin-6-yl)-1- isopropylpiperidine-4-carbonitrile | 513.4 |
| B118 | 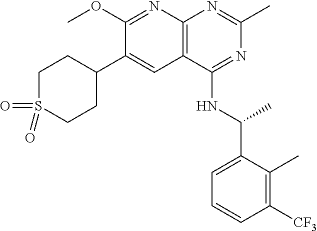 [SMILES: COc1nc2nc(C)nc(N[C@H](C)c3cccc(C(F)(F)F)c3C)c2cc1C1CCS(=O)(=O)CC1] | (R)-4-(7-methoxy-2-methyl-4-((1-(2-methyl-3- (trifluoromethyl)phenyl)ethyl)amino)pyrido[2,3- d]pyrimidin-6-yl)tetrahydro-2H-thiopyran 1,1- dioxide | 509.9 |
| B119 | 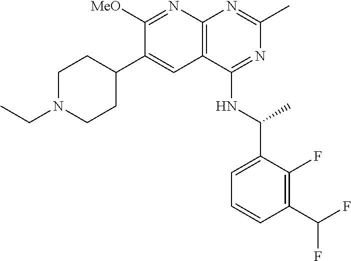 [SMILES: CCN1CCC(c2cc3c(N[C@H](C)c4cccc(C(F)F)c4F)nc(C)nc3nc2OC)CC1] | (R)-N-(1-(3-(difluoromethyl)-2- fluorophenyl)ethyl)-6-(1-ethylpiperidin-4-yl)-7- methoxy-2-methylpyrido[2,3-d]pyrimidin-4- amine | 474.5 |
| B120 | 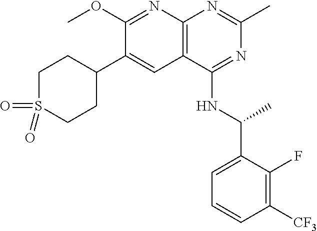 [SMILES: COc1nc2nc(C)nc(N[C@H](C)c3cccc(C(F)(F)F)c3F)c2cc1C1CCS(=O)(=O)CC1] | (R)-4-(4-((1-(2-fluoro-3- (trifluoromethyl)phenyl)ethyl)amino)-7-methoxy- 2-methylpyrido[2,3-d]pyrimidin-6-yl)tetrahydro- 2H-thiopyran 1,1-dioxide | 513.8 |
| B121 | 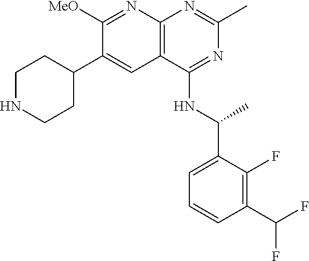 [SMILES: COc1nc2nc(C)nc(N[C@H](C)c3cccc(C(F)F)c3F)c2cc1C1CCNCC1] | (R)-N-(1-(3-(difluoromethyl)-2- fluorophenyl)ethyl)-7-methoxy-2-methyl-6- (piperidin-4-yl)pyrido[2,3-d]pyrimidin-4-amine | 446.4 |
| B122 | 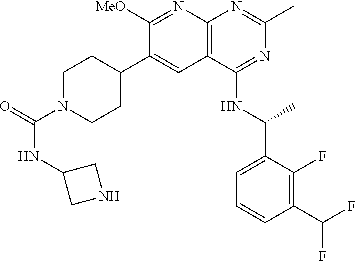 [SMILES: COc1nc2nc(C)nc(N[C@H](C)c3cccc(C(F)F)c3F)c2cc1C1CCN(C(=O)NC2CNC2)CC1] | (R)-N-(azetidin-3-yl)-4-(4-((1-(3- (difluoromethyl)-2-fluorophenyl)eth- yl)amino)-7-methoxy-2-methyl- pyrido[2,3-d]pyrimidin-6-yl)piperidine-1- carboxamide | 544.6 |
| B123 | 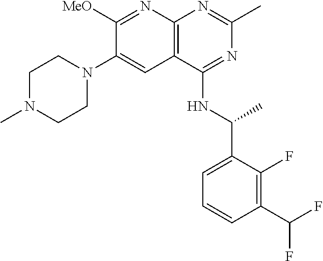 [SMILES: COc1nc2nc(C)nc(N[C@H](C)c3cccc(C(F)F)c3F)c2cc1N1CCN(C)CC1] | (R)-N-(1-(3-(difluoromethyl)-2- fluorophenyl)ethyl)-7-methoxy-2-methyl-6-(4- methylpiperazin-1-yl)pyrido[2,3-d]pyrimidin-4- amine | 475.9 |
| B124 | 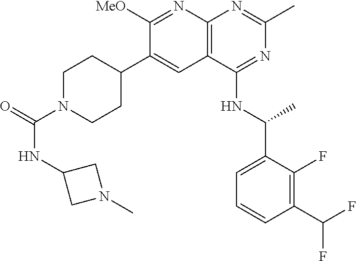 [SMILES: COc1nc2nc(C)nc(N[C@H](C)c3cccc(C(F)F)c3F)c2cc1C1CCN(C(=O)NC2CN(C)C2)CC1] | (R)-4-(4-((1-(3-(difluoromethyl)-2- fluorophenyl)ethyl)amino)-7-methoxy-2- methylpyrido[2,3-d]pyrimidin-6-yl)-N-(1- methylazetidin-3-yl)piperidine-1-carboxamide | 558.6 |
| B125 | 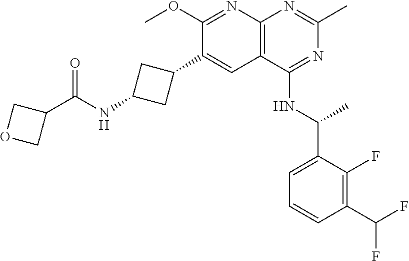 [SMILES: COc1nc2nc(C)nc(N[C@H](C)c3cccc(C(F)F)c3F)c2cc1[C@H]1C[C@@H](NC(=O)C2COC2)C1] | N-((1S,3s)-3-(4-(((R)-1-(3-(difluoromethyl)-2- fluorophenyl)ethyl)amino)-7-methoxy-2- methylpyrido[2,3-d]pyrimidin-6- yl)cyclobutyl)oxetane-3-carboxamide | 516.9 |
| B126 | 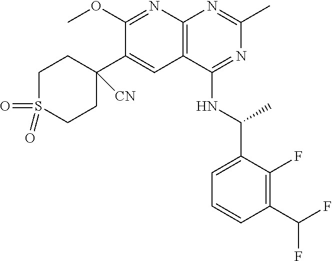 [SMILES: COc1nc2nc(C)nc(N[C@H](C)c3cccc(C(F)F)c3F)c2cc1C1(C#N)CCS(=O)(=O)CC1] | (R)-4-(4-((1-(3-(difluoromethyl)-2- fluorophenyl)ethyl)amino)-7-methoxy-2- methylpyrido[2,3-d]pyrimidin-6-yl)tetrahydro- 2H-thiopyran-4-carbonitrile 1,1-dioxide | 520.8 |
| B127 | 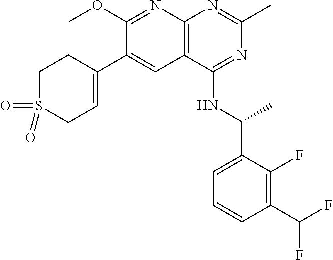 [SMILES: COc1nc2nc(C)nc(N[C@H](C)c3cccc(C(F)F)c3F)c2cc1C1=CCS(=O)(=O)CC1] | (R)-4-(4-((1-(3-(difluoromethyl)-2- fluorophenyl)ethyl)amino)-7-methoxy-2- methylpyrido[2,3-d]pyrimidin-6-yl)-3,6-dihydro- 2H-thiopyran 1,1-dioxide | 493.8 |
| B128 | 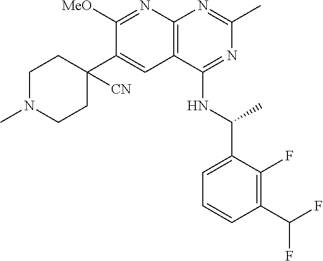 [SMILES: COc1nc2nc(C)nc(N[C@H](C)c3cccc(C(F)F)c3F)c2cc1C1(C#N)CCN(C)CC1] | (R)-4-(4-((1-(3-(difluoromethyl)-2- fluorophenyl)ethyl)amino)-7-methoxy-2- methylpyrido[2,3-d]pyrimidin-6-yl)-1- methylpiperidine-4-carbonitrile | 485.5 |
| B129 | 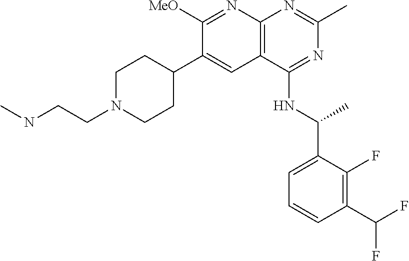 [SMILES: CNCCN1CCC(c2cc3c(N[C@H](C)c4cccc(C(F)F)c4F)nc(C)nc3nc2OC)CC1] | (R)-N-(1-(3-(difluoromethyl)-2- fluorophenyl)ethyl)-7-methoxy-2-methyl-6-(1-(2- (methyl-12-azaneyl)ethyl)piperidin-4- yl)pyrido[2,3-d]pyrimidin-4-amine | 503.5 |
| B130 | 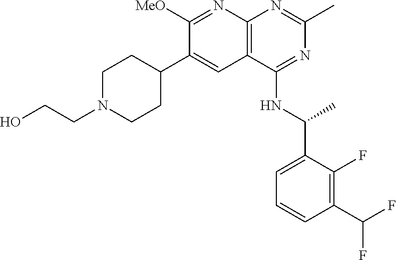 [SMILES: COc1nc2nc(C)nc(N[C@H](C)c3cccc(C(F)F)c3F)c2cc1C1CCN(CCO)CC1] | (R)-2-(4-(4-((1-(3-(difluoromethyl)-2- fluorophenyl)ethyl)amino)-7-methoxy-2- methylpyrido[2,3-d]pyrimidin-6-yl)piperidin-1- yl)ethan-1-ol | 490.4 |
| B131 | 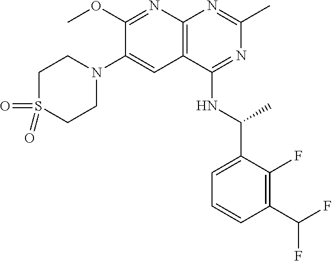 [SMILES: COc1nc2nc(C)nc(N[C@H](C)c3cccc(C(F)F)c3F)c2cc1N1CCS(=O)(=O)CC1] | (R)-4-(4-((1-(3-(difluoromethyl)-2- fluorophenyl)ethyl)amino)-7-methoxy-2- methylpyrido[2,3-d]pyrimidin-6-yl)thio- morpholine 1,1-dioxide | 496.8 |
| B132 | 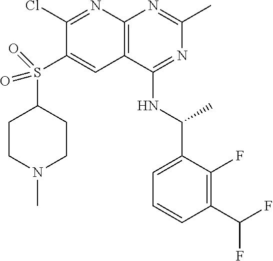 [SMILES: Cc1nc(N[C@H](C)c2cccc(C(F)F)c2F)c2cc(S(=O)(=O)C3CCN(C)CC3)c(Cl)nc2n1] | (R)-7-chloro-N-(1-(3-(difluoromethyl)-2- fluorophenyl)ethyl)-2-methyl-6-((1- methylpiperidin-4-yl)sulfonyl)pyrido[2,3- d]pyrimidin-4-amine | 528.9 |
| B133 | 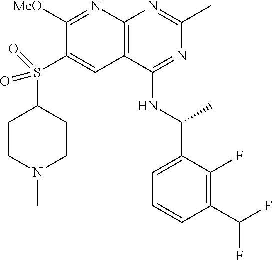 [SMILES: COc1nc2nc(C)nc(N[C@H](C)c3cccc(C(F)F)c3F)c2cc1S(=O)(=O)C1CCN(C)CC1] | (R)-N-(1-(3-(difluoromethyl)-2- fluorophenyl)ethyl)-7-methoxy-2-methyl-6-((1- methylpiperidin-4-yl)sulfonyl)pyrido[2,3- d]pyrimidin-4-amine | 524.4 |
| B134 | 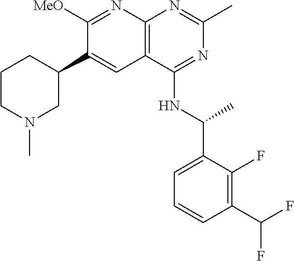 [SMILES: COc1nc2nc(C)nc(N[C@H](C)c3cccc(C(F)F)c3F)c2cc1[C@@H]1CCCN(C)C1] | N-((R)-1-(3-(difluoromethyl)-2-fluoro- phenyl)ethyl)-7-methoxy-2-methyl-6-((S)-1- methylpiperidin-3-yl)pyrido[2,3-d]pyrimidin-4- amine | 460.4 |
| B135 | 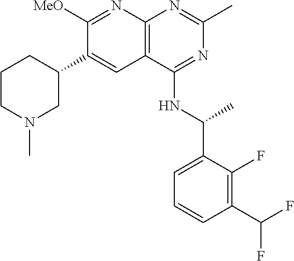 [SMILES: COc1nc2nc(C)nc(N[C@H](C)c3cccc(C(F)F)c3F)c2cc1[C@H]1CCCN(C)C1] | N-((R)-1-(3-(difluoromethyl)-2-fluoro- phenyl)ethyl)-7-methoxy-2-methyl-6-((R)-1- methylpiperidin-3-yl)pyrido[2,3-d]pyrimidin-4- amine | 460.4 |
| B136 | 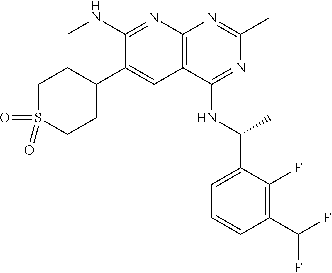 [SMILES: CNc1nc2nc(C)nc(N[C@H](C)c3cccc(C(F)F)c3F)c2cc1C1CCS(=O)(=O)CC1] | (R)-4-(4-((1-(3-(difluoromethyl)-2- fluorophenyl)ethyl)amino)-2-methyl-7- (methylamino)pyrido[2,3-d]pyrimidin-6- yl)tetrahydro-2H-thiopyran 1,1-dioxide | 494.8 |
| B137 | 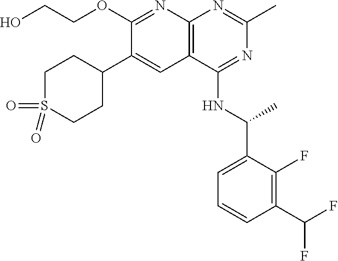 [SMILES: Cc1nc(N[C@H](C)c2cccc(C(F)F)c2F)c2cc(C3CCS(=O)(=O)CC3)c(OCCO)nc2n1] | (R)-4-(4-((1-(3-(difluoromethyl)-2-fluoro- phenyl)ethyl)amino)-7-(2-hydroxyethoxy)-2- methylpyrido[2,3-d]pyrimidin-6-yl)tetrahydro- 2H-thiopyran 1,1-dioxide | 525.8 |
| B138 | 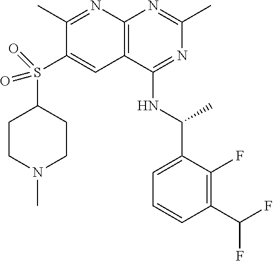 [SMILES: Cc1nc(N[C@H](C)c2cccc(C(F)F)c2F)c2cc(S(=O)(=O)C3CCN(C)CC3)c(C)nc2n1] | (R)-N-(1-(3-(difluoromethyl)-2- fluorophenyl)ethyl)-2,7-dimethyl-6-((1- methylpiperidin-4-yl)sulfonyl)pyrido[2,3- d]pyrimidin-4-amine | 494.4 |
| B139 | 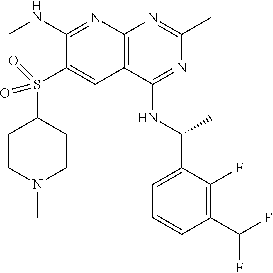 [SMILES: CNc1nc2nc(C)nc(N[C@H](C)c3cccc(C(F)F)c3F)c2cc1S(=O)(=O)C1CCN(C)CC1] | (R)-N4-(1-(3-(difluoromethyl)-2- fluorophenyl)ethyl)-N7,2-dimethyl-6-((1- methylpiperidin-4-yl)sulfonyl)pyrido[2,3- d]pyrimidine-4,7-diamine | 523.5 |
| B140 | 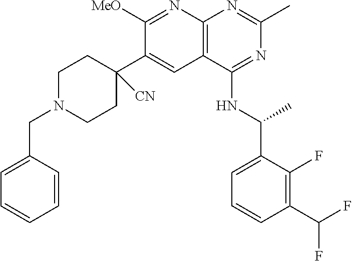 [SMILES: COc1nc2nc(C)nc(N[C@H](C)c3cccc(C(F)F)c3F)c2cc1C1(C#N)CCN(Cc2ccccc2)CC1] | (R)-1-benzyl-4-(4-((1-(3-(difluoromethyl)-2- fluorophenyl)ethyl)amino)-7-methoxy-2- methylpyrido[2,3-d]pyrimidin-6-yl)piperidine-4- carbonitrile | 561.5 |
| B141 | 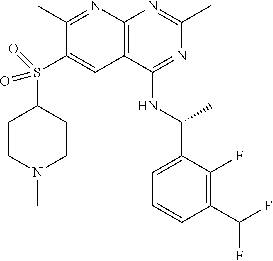 [SMILES: Cc1nc(N[C@H](C)c2cccc(C(F)F)c2F)c2cc(S(=O)(=O)C3CCN(C)CC3)c(C)nc2n1] | (R)-N-(1-(3-(difluoromethyl)-2- fluorophenyl)ethyl)-2,7-dimethyl-6-((1- methylpiperidin-4-yl)sulfonyl)pyrido[2,3- d]pyrimidin-4-amine | 508.4 |
| B142 | 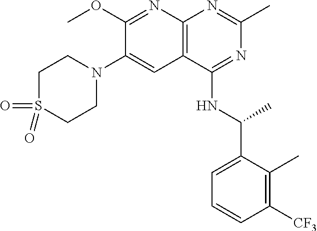 [SMILES: COc1nc2nc(C)nc(N[C@H](C)c3cccc(C(F)(F)F)c3C)c2cc1N1CCS(=O)(=O)CC1] | (R)-4-(7-methoxy-2-methyl-4-((1-(2-methyl-3- (trifluoromethyl)phenyl)ethyl)amino)pyrido[2,3- d]pyrimidin-6-yl)thiomorpholine 1,1-dioxide | 510.5 |
| B143 | 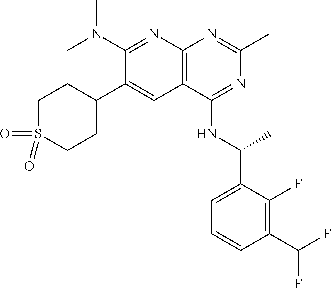 [SMILES: Cc1nc(N[C@H](C)c2cccc(C(F)F)c2F)c2cc(C3CCS(=O)(=O)CC3)c(N(C)C)nc2n1] | (R)-4-(4-((1-(3-(difluoromethyl)-2-fluoro- phenyl)ethyl)amino)-7-(dimethylamino)-2- methylpyrido[2,3-d]pyrimidin-6-yl)tetrahydro- 2H-thiopyran 1,1-dioxide | 508.8 |
| B144 | 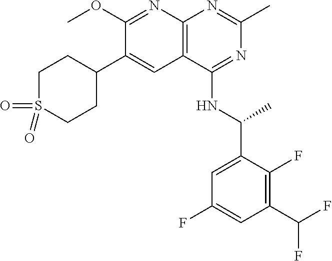 [SMILES: COc1nc2nc(C)nc(N[C@H](C)c3cc(F)cc(C(F)F)c3F)c2cc1C1CCS(=O)(=O)CC1] | (R)-4-(4-((1-(3-(difluoromethyl)-2,5- difluorophenyl)ethyl)amino)-7-methoxy-2- methylpyrido[2,3-d]pyrimidin-6-yl)tetrahydro- 2H-thiopyran 1,1-dioxide | 513.9 |
| B145 | 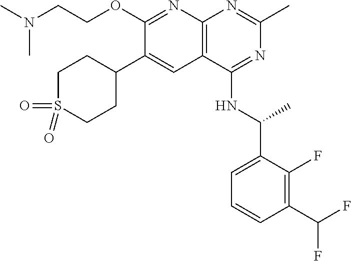 [SMILES: Cc1nc(N[C@H](C)c2cccc(C(F)F)c2F)c2cc(C3CCS(=O)(=O)CC3)c(OCCN(C)C)nc2n1] | (R)-4-(4-((1-(3-(difluoromethyl)-2- fluorophenyl)ethyl)amino)-7-(2- (dimethylamino)ethoxy)-2-methylpyrido[2,3- d]pyrimidin-6-yl)tetrahydro-2H-thiopyran 1,1- dioxide | 552.9 |
| B146 | 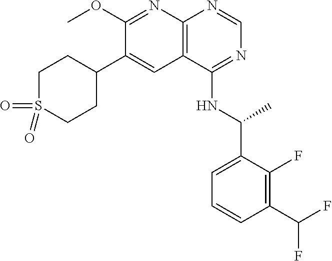 [SMILES: COc1nc2ncnc(N[C@H](C)c3cccc(C(F)F)c3F)c2cc1C1CCS(=O)(=O)CC1] | (R)-4-(4-((1-(3-(difluoromethyl)-2-fluoro- phenyl)ethyl)amino)-7-methoxypyrido[2,3- d]pyrimidin-6-yl)tetrahydro-2H-thiopyran 1,1- dioxide | 481.4 |
| B147 | 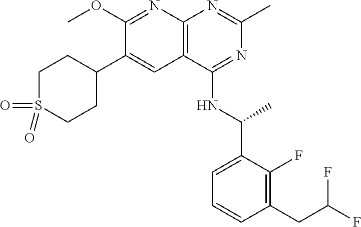 [SMILES: COc1nc2nc(C)nc(N[C@H](C)c3cccc(CC(F)F)c3F)c2cc1C1CCS(=O)(=O)CC1] | (R)-4-(4-((1-(3-(2,2-difluoroethyl)-2- fluorophenyl)ethyl)amino)-7-methoxy-2- methylpyrido[2,3-d]pyrimidin-6-yl)tetrahydro- 2H-thiopyran 1,1-dioxide | 509.8 |
| B148 | 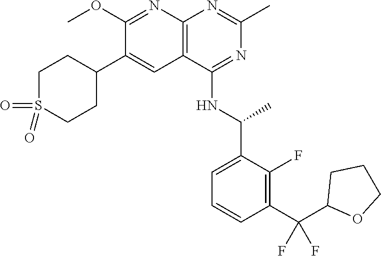 [SMILES: COc1nc2nc(C)nc(N[C@H](C)c3cccc(C(F)(F)C4CCCO4)c3F)c2cc1C1CCS(=O)(=O)CC1] | 4-(4-(((R)-1-(3-(difluoro((S)-tetrahydrofuran-2- yl)methyl)-2-fluorophenyl)ethyl)amino)-7- methoxy-2-methylpyrido[2,3-d]pyrimidin-6- yl)tetrahydro-2H-thiopyran 1,1-dioxide | 565.9 |
| B149 | 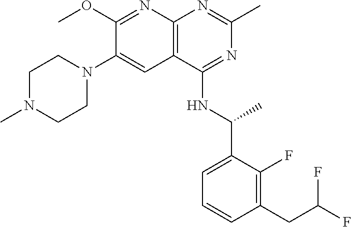 [SMILES: COc1nc2nc(C)nc(N[C@H](C)c3cccc(CC(F)F)c3F)c2cc1N1CCN(C)CC1] | (R)-N-(1-(3-(2,2-difluoroethyl)-2- fluorophenyl)ethyl)-7-methoxy-2-methyl-6-(4- methylpiperazin-1-yl)pyrido[2,3-d]pyrimidin-4- amine | 475.5 |
| B150 | 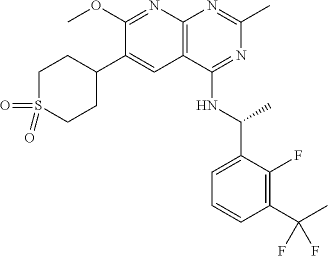 [SMILES: COc1nc2nc(C)nc(N[C@H](C)c3cccc(C(C)(F)F)c3F)c2cc1C1CCS(=O)(=O)CC1] | (R)-4-(4-((1-(3-(1,1-difluoroethyl)-2- fluorophenyl)ethyl)amino)-7-methoxy-2- methylpyrido[2,3-d]pyrimidin-6-yl)tetrahydro- 2H-thiopyran 1,1-dioxide | 509.9 |
| B151 | 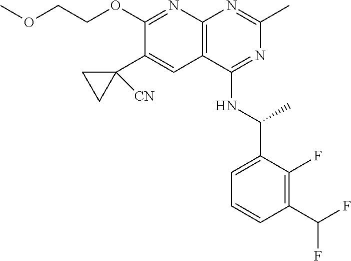 [SMILES: COCCOc1nc2nc(C)nc(N[C@H](C)c3cccc(C(F)F)c3F)c2cc1C1(C#N)CC1] | (R)-1-(4-((1-(3-(difluoromethyl)-2-fluoro- phenyl)ethyl)amino)-7-(2-methoxyethoxy)- 2-methylpyrido[2,3-d]pyrimidin-6- yl)cyclopropane-1-carbonitrile | 472.9 |
| B152 | 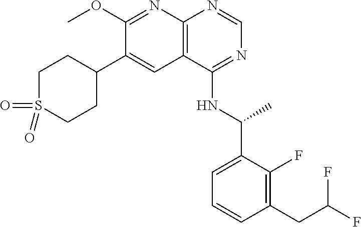 [SMILES: COc1nc2ncnc(N[C@H](C)c3cccc(CC(F)F)c3F)c2cc1C1CCS(=O)(=O)CC1] | (R)-4-(4-((1-(3-(2,2-difluoroethyl)-2-fluoro- phenyl)ethyl)amino)-7-methoxypyrido[2,3- d]pyrimidin-6-yl)tetrahydro-2H-thiopyran 1,1- dioxide | 495.8 |
| B153 | 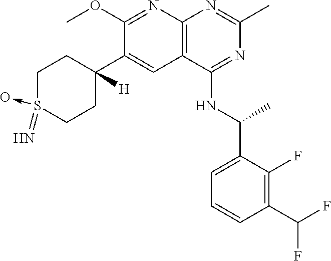 | 4-(4-(((R)-1-(3-(difluoromethyl)-2- fluorophenyl)ethyl)amino)-7-methoxy-2- methylpyrido[2,3-d]pyrimidin-6-yl)-1- iminohexahydro-1λ6-thiopyran 1-oxide | 494.8 |
| B154 | 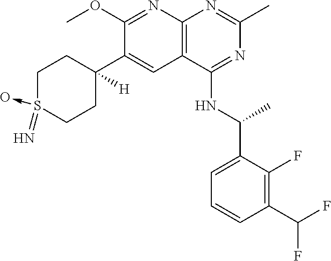 | (R)-4-(4-((1-(3-(difluoromethyl)-2- fluorophenyl)ethyl)amino)-7-methoxy-2- methylpyrido[2,3-d]pyrimidin-6-yl)-1- iminohexahydro-1λ6-thiopyran 1-oxide | 494.8 |
| B155 | 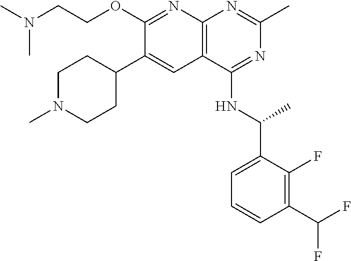 [SMILES: Cc1nc(N[C@H](C)c2cccc(C(F)F)c2F)c2cc(C3CCN(C)CC3)c(OCCN(C)C)nc2n1] | (R)-1-(4-((1-(3-(difluoromethyl)-2- fluorophenyl)ethyl)amino)-7-(2- (dimethylamino)ethoxy)-2-methylpyrido[2,3- d]pyrimidin-6-yl)cyclopropane-1-carbonitrile | 517.5 |
| B156 | 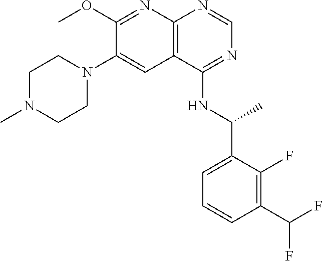 [SMILES: COc1nc2ncnc(N[C@H](C)c3cccc(C(F)F)c3F)c2cc1N1CCN(C)CC1] | (R)-N-(1-(3-(difluoromethyl)-2- fluorophenyl)ethyl)-7-methoxy-6-(4- methylpiperazin-1-yl)pyrido[2,3-d]pyrimidin-4- amine | 447.3 |
| B157 | 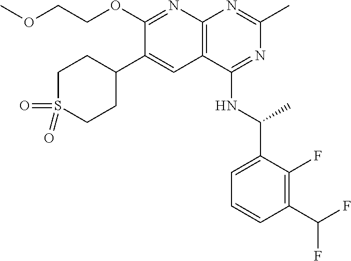 [SMILES: COCCOc1nc2nc(C)nc(N[C@H](C)c3cccc(C(F)F)c3F)c2cc1C1CCS(=O)(=O)CC1] | (R)-4-(4-((1-(3-(difluoromethyl)-2- fluorophenyl)ethyl)amino)-7-(2-methoxyethoxy)- 2-methylpyrido[2,3-d]pyrimidin-6-yl)tetrahydro- 2H-thiopyran 1,1-dioxide | 539.8 |
| B158 | 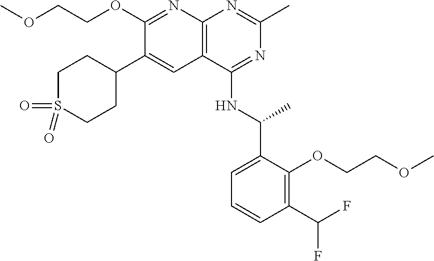 | (R)-4-(4-((1-(3-(difluoromethyl)-2-(2- methoxyethoxy)phenyl)ethyl)amino)-7-(2- methoxyethoxy)-2-methylpyrido[2,3-d]pyrimi- din-6-yl)tetrahydro-2H-thiopyran 1,1-dioxide | 595.9 |
| B159 | 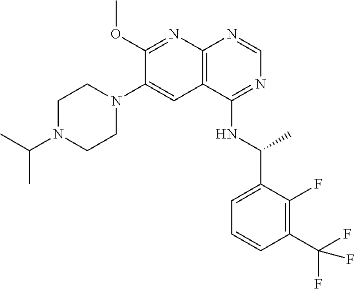 [SMILES: COc1nc2ncnc(N[C@H](C)c3cccc(C(F)(F)F)c3F)c2cc1N1CCN(C(C)C)CC1] | (R)-N-(1-(2-fluoro-3- (trifluoromethyl)phenyl)ethyl)-6-(4- isopropylpiperazin-1-yl)-7-methoxypyrido[2,3- d]pyrimidin-4-amine | 493.4 |
| B160 | 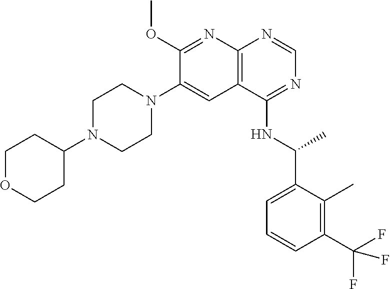 [SMILES: COc1nc2ncnc(N[C@H](C)c3cccc(C(F)(F)F)c3C)c2cc1N1CCN(C2CCOCC2)CC1] | (R)-7-methoxy-N-(1-(2-methyl-3- (trifluoromethyl)phenyl)ethyl)-6-(4-(tetrahydro- 2H-pyran-4-y1)piperazin-1-yl)pyrido[2,3- d]pyrimidin-4-amine | 531.4 |
| B161 | 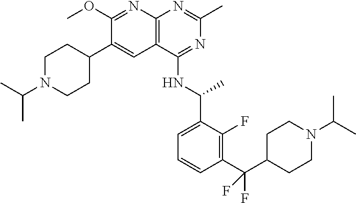 [SMILES: COc1nc2nc(C)nc(N[C@H](C)c3cccc(C(F)(F)C4CCN(C(C)C)CC4)c3F)c2cc1C1CCN(C(C)C)CC1] | (R)-N-(1-(3-(difluoro(1-isopropylpiperidin-4- yl)methyl)-2-fluorophenyl)ethyl)-6-(1- isopropylpiperidin-4-yl)-7-methoxy-2- methylpyrido[2,3-d]pyrimidin-4-amine | 613.6 |
| B162 | 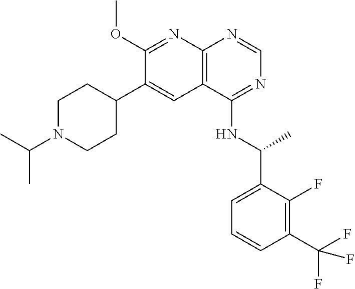 [SMILES: COc1nc2ncnc(N[C@H](C)c3cccc(C(F)(F)F)c3F)c2cc1C1CCN(C(C)C)CC1] | (R)-N-(1-(2-fluoro-3- (trifluoromethyl)phenyl)ethyl)-6-(1- isopropylpiperidin-4-yl)-7-methoxypyrido[2,3- d]pyrimidin-4-amine | 492.4 |
| B163 | 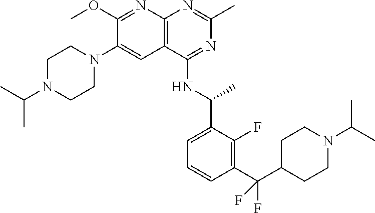 [SMILES: COc1nc2nc(C)nc(N[C@H](C)c3cccc(C(F)(F)C4CCN(C(C)C)CC4)c3F)c2cc1N1CCN(C(C)C)CC1] | (R)-N-(1-(3-(difluoro(1-isopropylpiperidin-4- yl)methyl)-2-fluorophenyl)ethyl)-6-(4- isopropylpiperazin-1-yl)-7-methoxy-2- methylpyrido[2,3-d]pyrimidin-4-amine | 614.5 |
| B164 | 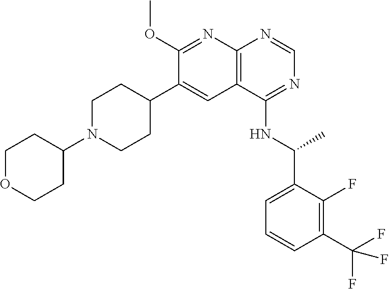 [SMILES: COc1nc2ncnc(N[C@H](C)c3cccc(C(F)(F)F)c3F)c2cc1C1CCN(C2CCOCC2)CC1] | (R)-N-(1-(2-fluoro-3- (trifluoromethyl)phenyl)ethyl)-7-methoxy-6-(1- (tetrahydro-2H-pyran-4-yl)piperidin-4- yl)pyrido[2,3-d]pyrimidin-4-amine | 534.5 |
| B165 | 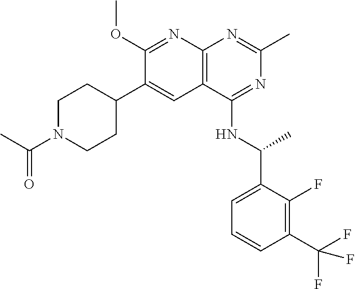 [SMILES: COc1nc2nc(C)nc(N[C@H](C)c3cccc(C(F)(F)F)c3F)c2cc1C1CCN(C(C)=O)CC1] | (R)-1-(4-(4-((1-(2-fluoro-3- (trifluoromethyl)phenyl)ethyl)amino)-7-methoxy- 2-methylpyrido[2,3-d]pyrimidin-6-yl)piperidin-1- yl)ethan-1-one | 506.8 |
| B166 | 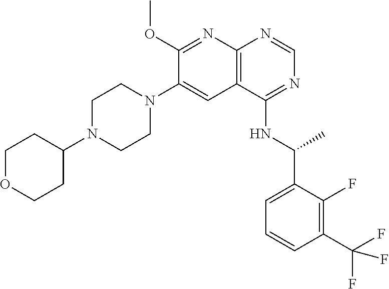 [SMILES: COc1nc2ncnc(N[C@H](C)c3cccc(C(F)(F)F)c3F)c2cc1N1CCN(C2CCOCC2)CC1] | (R)-N-(1-(2-fluoro-3- (trifluoromethyl)phenyl)ethyl)-7-methoxy-6-(4- (tetrahydro-2H-pyran-4-yl)piperazin-1- yl)pyrido[2,3-d]pyrimidin-4-amine | 535.4 |
| B167 | 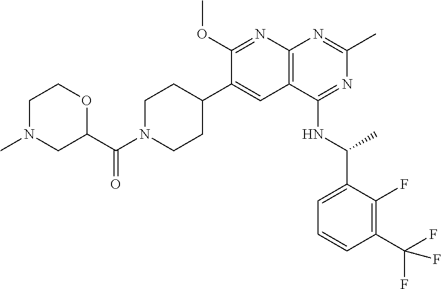 [SMILES: COc1nc2nc(C)nc(N[C@H](C)c3cccc(C(F)(F)F)c3F)c2cc1C1CCN(C(=O)C2CN(C)CCO2)CC1] | (4-(4-(((R)-1-(2-fluoro-3-(trifluoro- methyl)phenyl)ethyl)amino)-7-methoxy- 2-methylpyrido[2,3-d]pyrimidin-6-yl)piperidin- 1-yl)(4-methylmorpholin-2-yl)methanone | 591.4 |
| B168 | 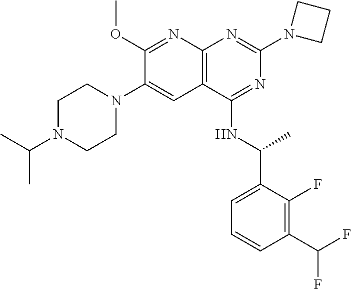 [SMILES: COc1nc2nc(N3CCC3)nc(N[C@H](C)c3cccc(C(F)F)c3F)c2cc1N1CCN(C(C)C)CC1] | (R)-2-(azetidin-1-yl)-N-(1-(3- (difluoromethyl)-2-fluorophenyl)ethyl)- 6-(4-isopropylpiperazin-1-yl)-7- methoxypyrido[2,3-d]pyrimidin-4-amine | 530.45 |
| B169 | 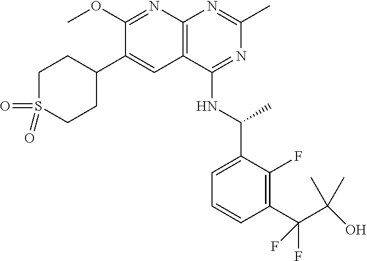 [SMILES: COc1nc2nc(C)nc(N[C@H](C)c3cccc(C(F)(F)C(C)(C)O)c3F)c2cc1C1CCS(=O)(=O)CC1] | (R)-4-(4-((1-(3-(1,1-difluoro-2-hydroxy-2- methylpropyl)-2-fluorophenyl)ethyl)amino)-7- methoxy-2-methylpyrido[2,3-d]pyrimidin-6- yl)thiomorpholine 1,1-dioxide | 554.4 |
| B170 | 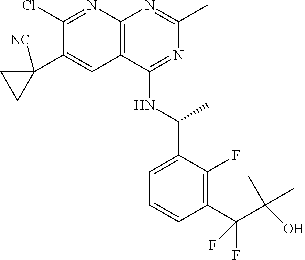 [SMILES: Cc1nc(N[C@H](C)c2cccc(C(F)(F)C(C)(C)O)c2F)c2cc(C3(C#N)CC3)c(Cl)nc2n1] | (R)-1-(7-chloro-4-((1-(3-(1,1- difluoro-2-hydroxy-2-methylpropyl)- 2-fluorophenyl)ethyl)amino)-2-methyl- pyrido[2,3-d]pyrimidin-6-yl)cyclopropane- 1-carbonitrile | 490.3 |
| B171 | 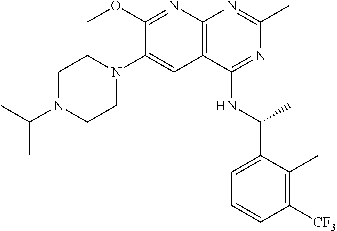 [SMILES: COc1nc2nc(C)nc(N[C@H](C)c3cccc(C(F)(F)F)c3C)c2cc1N1CCN(C(C)C)CC1] | (R)-6-(4-isopropylpiperazin-1-yl)-7-methoxy-2- methyl-N-(1-(2-methyl-3- (trifluoromethyl)phenyl)ethyl)pyrido[2,3- d]pyrimidin-4-amine | 503.3 |
| B172 | 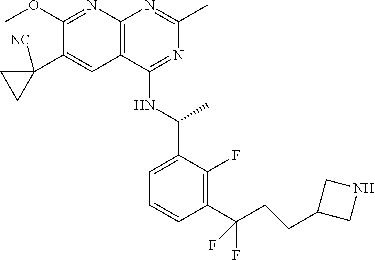 [SMILES: COc1nc2nc(C)nc(N[C@H](C)c3cccc(C(F)(F)CCC4CNC4)c3F)c2cc1C1(C#N)CC1] | (R)-1-(4-((1-(3-(3-(azetidin-3-yl)-1,1- difluoropropyl)-2-fluorophenyl)ethyl)amino)-7- methoxy-2-methylpyrido[2,3-d]pyrimidin-6- yl)cyclopropane-1-carbonitrile | 511.35 |
| B173 | 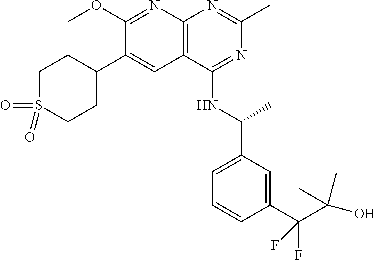 [SMILES: COc1nc2nc(C)nc(N[C@H](C)c3cccc(C(F)(F)C(C)(C)O)c3)c2cc1C1CCS(=O)(=O)CC1] | (R)-4-(4-((1-(3-(1,1-difluoro-2-hydroxy-2- methylpropyl)phenyl)ethyl)amino)-7- methoxy-2-methylpyrido[2,3-d]pyrimidin- 6-yl)tetrahydro-2H-thiopyran 1,1-dioxide | 535.6 |
| B174 | 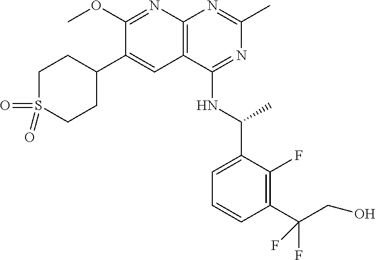 [SMILES: COc1nc2nc(C)nc(N[C@H](C)c3cccc(C(F)(F)CO)c3F)c2cc1C1CCS(=O)(=O)CC1] | (R)-4-(4-((1-(3-(1,1-difluoro-2-hydroxyethyl)- 2-fluorophenyl)ethyl)amino)-7-methoxy-2- methylpyrido[2,3-d]pyrimidin-6-yl)tetrahydro- 2H-thiopyran 1,1-dioxide | 525.6 |
| B175 | 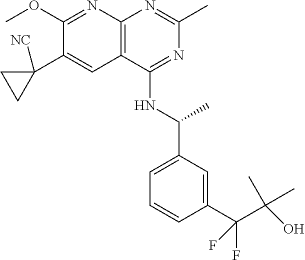 [SMILES: COc1nc2nc(C)nc(N[C@H](C)c3cccc(C(F)(F)C(C)(C)O)c3)c2cc1C1(C#N)CC1] | (R)-1-(4-((1-(3-(1,1-difluoro-2-hydroxy-2- methylpropyl)phenyl)ethyl)amino)-7-methoxy- 2-methylpyrido[2,3-d]pyrimidin-6- yl)cyclopropane-1-carbonitrile | 468.6 |
| B176 | 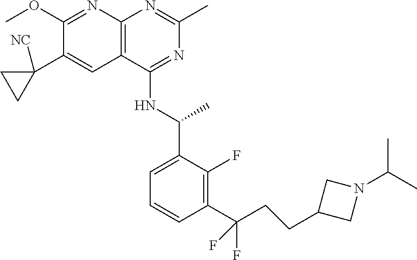 [SMILES: COc1nc2nc(C)nc(N[C@H](C)c3cccc(C(F)(F)CCC4CN(C(C)C)C4)c3F)c2cc1C1(C#N)CC1] | (R)-1-(4-((1-(3-(1,1-difluoro-3-(1- isopropylazetidin-3-yl)propyl)-2- fluorophenyl)ethyl)amino)-7-methoxy-2- methylpyrido[2,3-d]pyrimidin-6- yl)cyclopropane-1-carbonitrile | 553.4 |
| B177 | 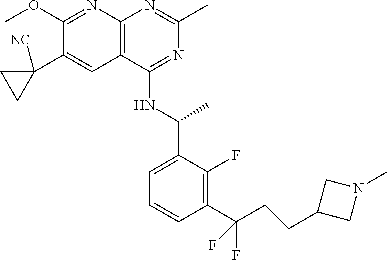 [SMILES: COc1nc2nc(C)nc(N[C@H](C)c3cccc(C(F)(F)CCC4CN(C)C4)c3F)c2cc1C1(C#N)CC1] | (R)-1-(4-((1-(3-(1,1-difluoro-3-(1- methylazetidin-3-yl)propyl)-2- fluorophenyl)ethyl)amino)-7- methoxy-2-methylpyrido[2,3-d]pyrimidin-6- yl)cyclopropane-1-carbonitrile | 525.3 |
| B178 | 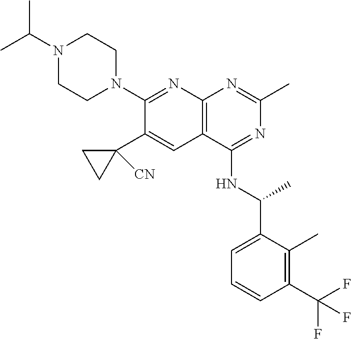 [SMILES: Cc1nc(N[C@H](C)c2cccc(C(F)(F)F)c2C)c2cc(C3(C#N)CC3)c(N3CCN(C(C)C)CC3)nc2n1] | (R)-1-(7-(4-isopropylpiperazin-1-yl)- 2-methyl-4-((1-(2-methyl-3-(tri- fluoromethyl)phenyl)ethyl)amino)pyrido[2,3- d]pyrimidin-6-yl)cyclopropane-1-carbonitrile | 538.4 |
| B179 | 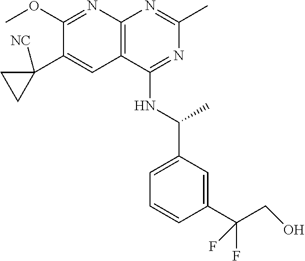 [SMILES: COc1nc2nc(C)nc(N[C@H](C)c3cccc(C(F)(F)CO)c3)c2cc1C1(C#N)CC1] | (R)-1-(4-((1-(3-(1,1-difluoro-2-hydroxy- ethyl)phenyl)ethyl)amino)-7-methoxy-2- methylpyrido[2,3-d]pyrimidin-6- yl)cyclopropane-1-carbonitrile | 440.6 |
| B180 | 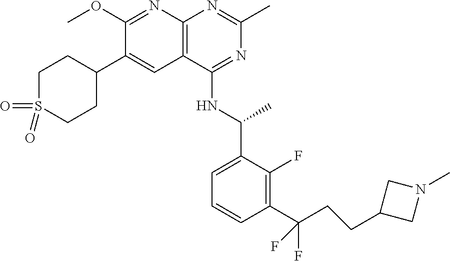 [SMILES: COc1nc2nc(C)nc(N[C@H](C)c3cccc(C(F)(F)CCC4CN(C)C4)c3F)c2cc1C1CCS(=O)(=O)CC1] | (R)-4-(4-((1-(3-(1,1-difluoro-3-(1- methylazetidin-3-yl)propyl)-2- fluorophenyl)ethyl)amino)-7-methoxy- 2-methylpyrido[2,3-d]pyrimidin-6- yl)tetrahydro-2H-thiopyran 1,1-dioxide | 592.4 |
| B181 | 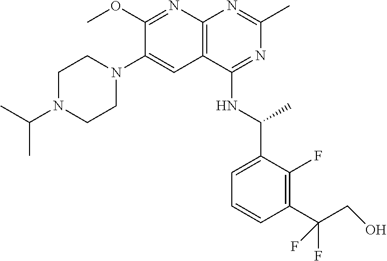 [SMILES: COc1nc2nc(C)nc(N[C@H](C)c3cccc(C(F)(F)CO)c3F)c2cc1N1CCN(C(C)C)CC1] | (R)-2,2-difluoro-2-(2-fluoro-3-(1-((6-(4- isopropylpiperazin-1-yl)-7-methoxy-2- methylpyrido[2,3-d]pyrimidin-4- yl)amino)ethyl)phenyl)ethan-1-ol | 519.3 |
| B182 | 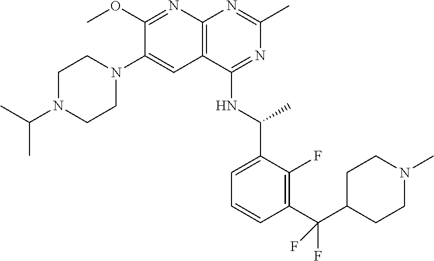 [SMILES: COc1nc2nc(C)nc(N[C@H](C)c3cccc(C(F)(F)C4CCN(C)CC4)c3F)c2cc1N1CCN(C(C)C)CC1] | (R)-N-(1-(3-(difluoro(1-methylpiperidin-4- yl)methyl)-2-fluorophenyl)ethyl)-6-(4- isopropylpiperazin-1-yl)-7-methoxy-2- methylpyrido[2,3-d]pyrimidin-4-amine | 586.5 |
| B183 | 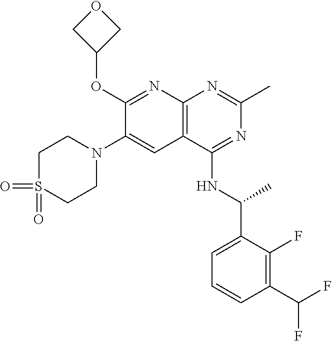 [SMILES: Cc1nc(N[C@H](C)c2cccc(C(F)F)c2F)c2cc(N3CCS(=O)(=O)CC3)c(OC3COC3)nc2n1] | (R)-4-(4-((1-(3-(difluoromethyl)-2- fluorophenyl)ethyl)amino)-2-methyl-7- (oxetan-3-yloxy)pyrido[2,3-d]pyrimidin- 6-yl)thiomorpholine 1,1-dioxide | 538.4 |
| B184 | 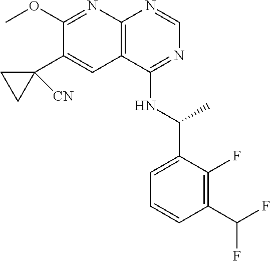 [SMILES: COc1nc2ncnc(N[C@H](C)c3cccc(C(F)F)c3F)c2cc1C1(C#N)CC1] | (R)-1-(4-((1-(3-(difluoromethyl)-2-fluoro- phenyl)ethyl)amino)-7-methoxypyrido[2,3- d]pyrimidin-6-yl)cyclopropane-1-carbonitrile | 414.7 |
| B185 | 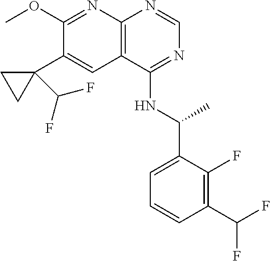 [SMILES: COc1nc2ncnc(N[C@H](C)c3cccc(C(F)F)c3F)c2cc1C1(C(F)F)CC1] | (R)-N-(1-(3-(difluoromethyl)-2- fluorophenyl)ethyl)-6-(1- (difluoromethyl)cyclopropyl)-7- methoxypyrido[2,3-d]pyrimidin-4-amine | 439.65 |
| B186 | 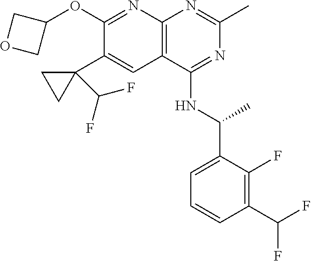 [SMILES: Cc1nc(N[C@H](C)c2cccc(C(F)F)c2F)c2cc(C3(C(F)F)CC3)c(OC3COC3)nc2n1] | (R)-N-(1-(3-(difluoromethyl)-2- fluorophenyl)ethyl)-6-(1-(difluoro- methyl)cyclopropyl)-2-methyl-7-(oxetan- 3-yloxy)pyrido[2,3-d]pyrimidin-4-amine | 495.8 |
| B187 | 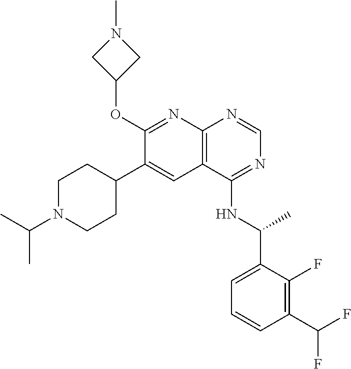 [SMILES: CC(C)N1CCC(c2cc3c(N[C@H](C)c4cccc(C(F)F)c4F)ncnc3nc2OC2CN(C)C2)CC1] | (R)-N-(1-(3-(difluoromethyl)-2-fluoro- phenyl)ethyl)-6-(1-isopropylpiperidin-4-yl)- 7-((1-methylazetidin-3-yl)oxy)pyrido[2,3- d]pyrimidin-4-amine | 529.9 |
| B188 | 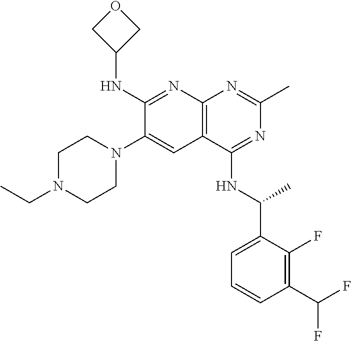 [SMILES: CCN1CCN(c2cc3c(N[C@H](C)c4cccc(C(F)F)c4F)nc(C)nc3nc2NC2COC2)CC1] | (R)-N4-(1-(3-(difluoromethyl)-2-fluoro- phenyl)ethyl)-6-(4-ethylpiperazin-1-yl)-2- methyl-N7-(oxetan-3-yl)pyrido[2,3- d]pyrimidine-4,7-diamine | 516.5 |
| B189 | 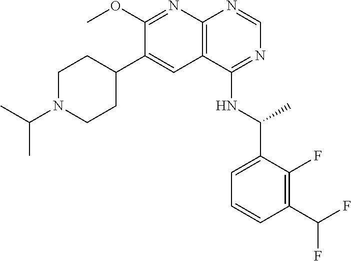 [SMILES: COc1nc2ncnc(N[C@H](C)c3cccc(C(F)F)c3F)c2cc1C1CCN(C(C)C)CC1] | (R)-N-(1-(3-(difluoromethyl)-2-fluoro- phenyl)ethyl)-6-(1-isopropylpiperidin-4-yl)- 7-methoxypyrido[2,3-d]pyrimidin-4-amine | 474.5 |
| B190 | 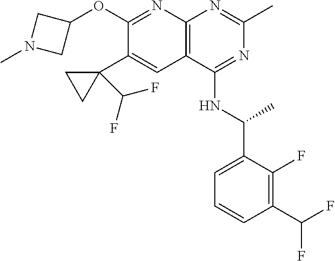 [SMILES: Cc1nc(N[C@H](C)c2cccc(C(F)F)c2F)c2cc(C3(C(F)F)CC3)c(OC3CN(C)C3)nc2n1] | (R)-N-(1-(3-(difluoromethyl)-2- fluorophenyl)ethyl)-6-(1-(difluoro- methyl)cyclopropyl)-2-methyl-7-((1- methylazetidin-3-yl)oxy)pyrido[2,3- d]pyrimidin-4-amine | 508.3 |
| B191 | 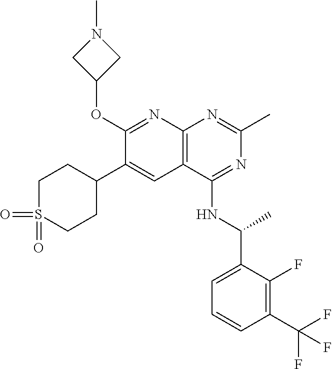 [SMILES: Cc1nc(N[C@H](C)c2cccc(C(F)(F)F)c2F)c2cc(C3CCS(=O)(=O)CC3)c(OC3CN(C)C3)nc2n1] | (R)-4-(4-((1-(3-(difluoromethyl)-2- fluorophenyl)ethyl)amino)-2-methyl-7-((1- methylazetidin-3-yl)oxy)pyrido[2,3- d]pyrimidin-6-yl)tetrahydro-2H-thiopyran 1,1-dioxide | 550.4 |
| B192 | 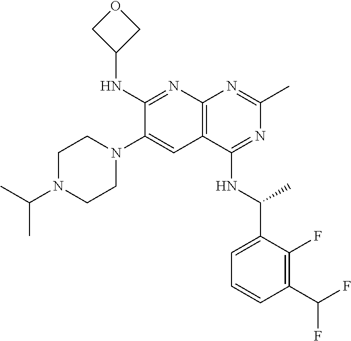 [SMILES: Cc1nc(N[C@H](C)c2cccc(C(F)F)c2F)c2cc(N3CCN(C(C)C)CC3)c(NC3COC3)nc2n1] | (R)-N4-(1-(3-(difluoromethyl)-2- fluorophenyl)ethyl)-6-(4-isopropylpiperazin- 1-yl)-2-methyl-N7-(oxetan-3-yl)pyrido[2,3- d]pyrimidine-4,7-diamine | 530.5 |
| B193 | 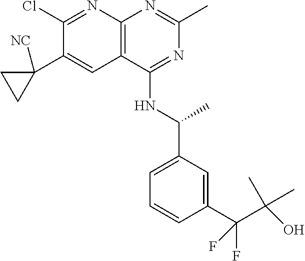 [SMILES: Cc1nc(N[C@H](C)c2cccc(C(F)(F)C(C)(C)O)c2)c2cc(C3(C#N)CC3)c(Cl)nc2n1] | (R)-1-(7-chloro-4-((1-(3-(1,1-difluoro-2- hydroxy-2-methylpropyl)phenyl)ethyl)ami- no)-2-methylpyrido[2,3-d]pyrimidin- 6-yl)cyclopropane-1-carbonitrile | 472.3 |
| B194 | 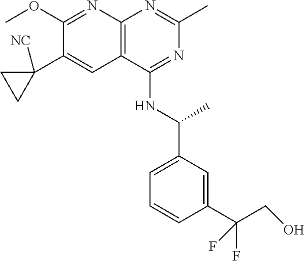 [SMILES: COc1nc2nc(C)nc(N[C@H](C)c3cccc(C(F)(F)CO)c3)c2cc1C1(C#N)CC1] | (R)-1-(7-chloro-4-((1-(3-(1,1-difluoro-2- hydroxyethyl)phenyl)ethyl)amino)-2- methylpyrido[2,3-d]pyrimidin-6- yl)cyclopropane-1-carbonitrile | 444.2 |
| B195 | 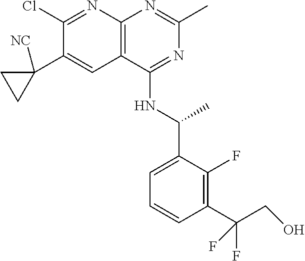 [SMILES: Cc1nc(N[C@H](C)c2cccc(C(F)(F)CO)c2F)c2cc(C3(C#N)CC3)c(Cl)nc2n1] | (R)-1-(7-chloro-4-((1-(3-(1,1-difluoro-2- hydroxyethyl)-2-fluorophenyl)ethyl)amino)- 2-methylpyrido[2,3-d]pyrimidin- 6-yl)cyclopropane-1-carbonitrile | 462.3 |
| B196 | 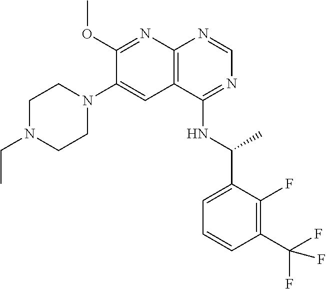 [SMILES: CCN1CCN(c2cc3c(N[C@H](C)c4cccc(C(F)(F)F)c4F)ncnc3nc2OC)CC1] | (R)-6-(4-ethylpiperazin-1-yl)-N-(1-(2-fluoro-3- (trifluoromethyl)phenyl)ethyl)-7- methoxypyrido[2,3-d]pyrimidin-4-amine | 479.3 |
| B197 | 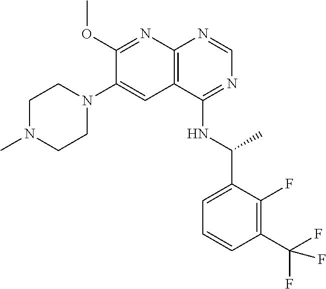 [SMILES: COc1nc2ncnc(N[C@H](C)c3cccc(C(F)(F)F)c3F)c2cc1N1CCN(C)CC1] | (R)-N-(1-(2-fluoro-3- (trifluoromethyl)phenyl)ethyl)-7-methoxy-6-(4- methylpiperazin-1-yl)pyrido[2,3-d]pyrimidin-4- amine | 465.3 |
| B198 | 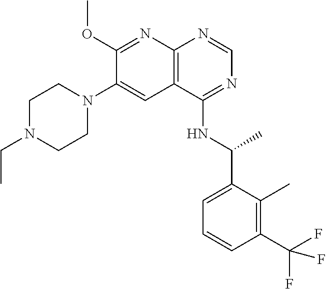 [SMILES: CCN1CCN(c2cc3c(N[C@H](C)c4cccc(C(F)(F)F)c4C)ncnc3nc2OC)CC1] | (R)-6-(4-ethylpiperazin-1-yl)-7-methoxy- N-(1-(2-methyl-3- (trifluoromethyl)phenyl)ethyl)pyrido[2,3- d]pyrimidin-4-amine | 475.4 |
| B199 | 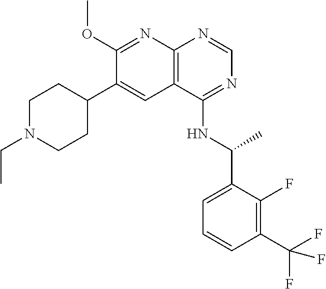 [SMILES: CCN1CCC(c2cc3c(N[C@H](C)c4cccc(C(F)(F)F)c4F)ncnc3nc2OC)CC1] | (R)-6-(1-ethylpiperidin-4-yl)-N-(1-(2-fluoro-3- (trifluoromethyl)phenyl)ethyl)-7- methoxypyrido[2,3-d]pyrimidin-4-amine | 478.4 |
| B200 | 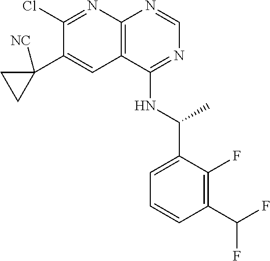 [SMILES: C[C@@H](Nc1ncnc2nc(Cl)c(C3(C#N)CC3)cc12)c1cccc(C(F)F)c1F] | (R)-1-(7-chloro-4-((1-(3-(difluoromethyl)-2- fluorophenyl)ethyl)amino)pyrido[2,3- d]pyrimidin-6-yl)cyclopropane-1-carbonitrile | 418.4 |
| B201 | 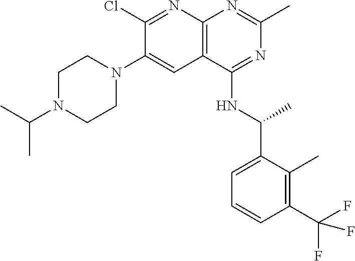 [SMILES: Cc1nc(N[C@H](C)c2cccc(C(F)(F)F)c2C)c2cc(N3CCN(C(C)C)CC3)c(Cl)nc2n1] | (R)-7-chloro-6-(4-isopropylpiperazin-1-yl)-2- methyl-N-(1-(2-methyl-3- (trifluoromethyl)phenyl)ethyl)pyrido[2,3- d]pyrimidin-4-amine | 507.3 |
| B202 | 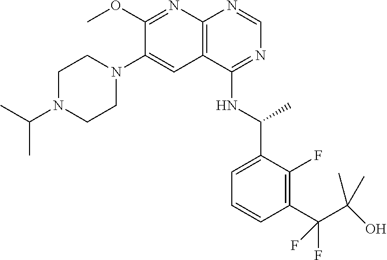 [SMILES: COc1nc2ncnc(N[C@H](C)c3cccc(C(F)(F)C(C)(C)O)c3F)c2cc1N1CCN(C(C)C)CC1] | (R)-1,1-difluoro-1-(3-(1-((6-(4-isopropyl- piperazin-1-yl)-7-methoxypyrido[2,3- d]pyrimidin-4-yl)amino)ethyl)phenyl)-2- methylpropan-2-ol | 515.5 |
| B203 | 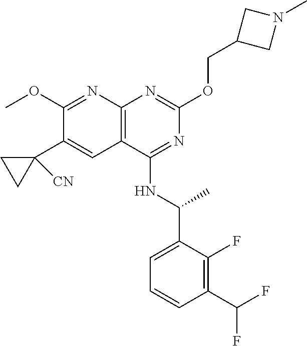 [SMILES: COc1nc2nc(OCC3CN(C)C3)nc(N[C@H](C)c3cccc(C(F)F)c3F)c2cc1C1(C#N)CC1] | (R)-1-(4-((1-(3-(difluoromethyl)-2- fluorophenyl)ethyl)amino)-7-methoxy-2-((1- methylazetidin-3-yl)methoxy)pyrido[2,3- d]pyrimidin-6-yl)cyclopropane-1-carbonitrile | 513.4 |
| B204 | 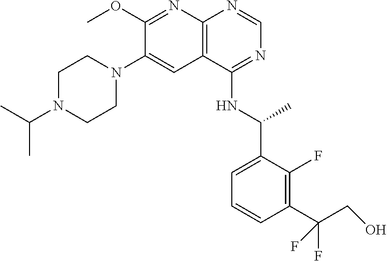 [SMILES: COc1nc2ncnc(N[C@H](C)c3cccc(C(F)(F)CO)c3F)c2cc1N1CCN(C(C)C)CC1] | (R)-2,2-difluoro-2-(2-fluoro-3-(1-((6-(4- isopropylpiperazin-1-yl)-7-methoxy- pyrido[2,3-d]pyrimidin-4-yl)amino)eth- yl)phenyl)ethan-1-ol | 505.5 |
| B205 | 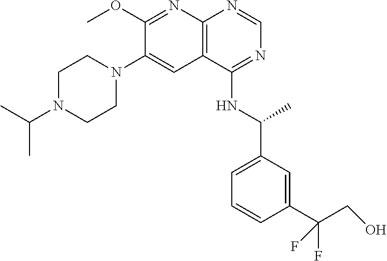 [SMILES: COc1nc2ncnc(N[C@H](C)c3cccc(C(F)(F)CO)c3)c2cc1N1CCN(C(C)C)CC1] | (R)-2,2-difluoro-2-(3-(1-((6-(4-isopropyl- 1piperazin--yl)-7-methoxypyrido[2,3- d]pyrimidin-4-yl)amino)ethyl)phe- nyl)ethan-1-ol | 487.4 |
| B206 | 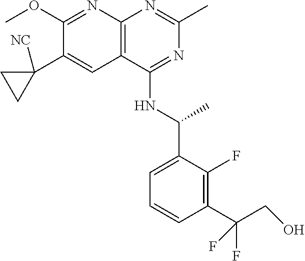 [SMILES: COc1nc2nc(C)nc(N[C@H](C)c3cccc(C(F)(F)CO)c3F)c2cc1C1(C#N)CC1] | (R)-1-(4-((1-(3-(1,1-difluoro-2-hydroxyethyl)- 2-fluorophenyl)ethyl)amino)-7-methoxy- 2-methylpyrido[2,3-d]pyrimidin-6- yl)cyclopropane-1-carbonitrile | 458.7 |
| B207 | 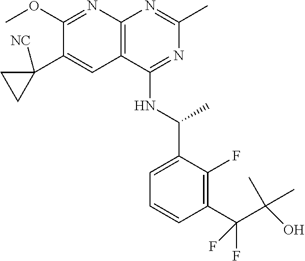 [SMILES: COc1nc2nc(C)nc(N[C@H](C)c3cccc(C(F)(F)C(C)(C)O)c3F)c2cc1C1(C#N)CC1] | (R)-1-(4-((1-(3-(1,1-difluoro-2-hydroxy-2- methylpropyl)-2-fluorophenyl)ethyl)amino)-7- methoxy-2-methylpyrido[2,3-d]pyrimidin-6- yl)cyclopropane-1-carbonitrile | 486.8 |
| B208 | 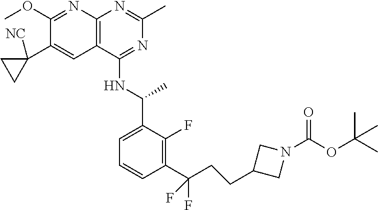 [SMILES: COc1nc2nc(C)nc(N[C@H](C)c3cccc(C(F)(F)CCC4CN(C(=O)OC(C)(C)C)C4)c3F)c2cc1C1(C#N)CC1] | tert-butyl (R)-3-(3-(3-(1-((6-(1-cyanocyclopro- pyl)-7-methoxy-2-methylpyrido[2,3-d]pyrimi- din-4-yl)amino)ethyl)-2-fluorophenyl)- 3,3-difluoropropyl)azetidine-1-carboxylate | 611.5 |
| B209 | 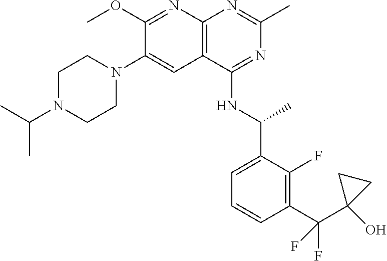 [SMILES: COc1nc2nc(C)nc(N[C@H](C)c3cccc(C(F)(F)C4(O)CC4)c3F)c2cc1N1CCN(C(C)C)CC1] | (R)-1-(difluoro(2-fluoro-3-(1-((6-(4- isopropylpiperazin-1-yl)-7-methoxy-2- methylpyrido[2,3-d]pyrimidin-4- yl)amino)ethyl)phenyl)methyl)cyclopropan-1-ol | 545.5 |
| B210 | 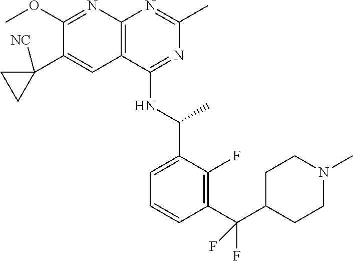 [SMILES: COc1nc2nc(C)nc(N[C@H](C)c3cccc(C(F)(F)C4CCN(C)CC4)c3F)c2cc1C1(C#N)CC1] | (R)-1-(4-((1-(3-(difluoro(1-methylpiperidin-4- yl)methyl)-2-fluorophenyl)ethyl)amino)-7- methoxy-2-methylpyrido[2,3-d]pyrimidin-6- yl)cyclopropane-1-carbonitrile | 525.5 |
| B211 | 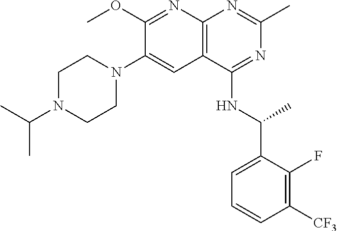 [SMILES: COc1nc2nc(C)nc(N[C@H](C)c3cccc(C(F)(F)F)c3F)c2cc1N1CCN(C(C)C)CC1] | (R)-N-(1-(2-fluoro-3- (trifluoromethyl)phenyl)ethyl)-6-(4- isopropylpiperazin-1-yl)-7-methoxy-2- methylpyrido[2,3-d]pyrimidin-4-amine | 507.4 |
| B212 | 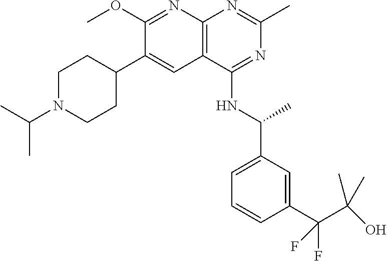 [SMILES: COc1nc2nc(C)nc(N[C@H](C)c3cccc(C(F)(F)C(C)(C)O)c3)c2cc1C1CCN(C(C)C)CC1] | (R)-1,1-difluoro-1-(3-(1-((6-(1- isopropylpiperidin-4-yl)-7-methoxy- 2-methylpyrido[2,3-d]pyrimidin-4- yl)amino)ethyl)phenyl)-2-methylpropan-2-ol | 528.5 |
| B213 | 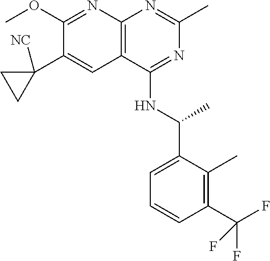 [SMILES: COc1nc2nc(C)nc(N[C@H](C)c3cccc(C(F)(F)F)c3C)c2cc1C1(C#N)CC1] | (R)-1-(7-methoxy-2-methyl- 4-((1-(2-methyl-3-(trifluoromethyl)phe- nyl)ethyl)amino)pyrido[2,3-d]pyrimidin-6- yl)cyclopropane-1-carbonitrile | 442.7 |
| B214 | 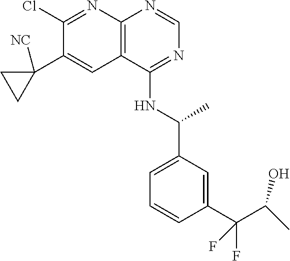 [SMILES: C[C@@H](Nc1ncnc2nc(Cl)c(C3(C#N)CC3)cc12)c1cccc(C(F)(F)[C@@H](C)O)c1] | 1-(7-chloro-4-(((R)-1-(3-((R)-1,1-difluoro-2- hydroxypropyl)phenyl)ethyl)amino)pyrido[2,3- d]pyrimidin-6-yl)cyclopropane-1-carbonitrile | 444.3 |
| B215 | 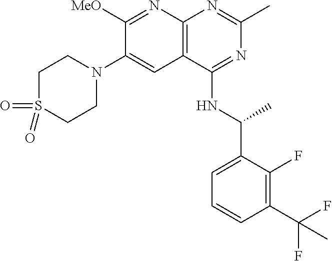 [SMILES: COc1nc2nc(C)nc(N[C@H](C)c3cccc(C(C)(F)F)c3F)c2cc1N1CCS(=O)(=O)CC1] | (R)-4-(4-((1-(3-(1,1-difluoroethyl)-2- fluorophenyl)ethyl)amino)-7-methoxy-2- methylpyrido[2,3-d]pyrimidin-6- yl)thiomorpholine 1,1-dioxide | 510.3 |
| B216 | 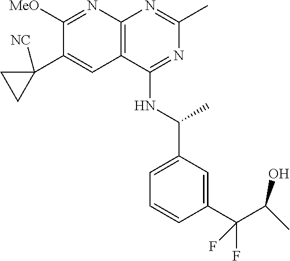 [SMILES: COc1nc2nc(C)nc(N[C@H](C)c3cccc(C(F)(F)[C@H](C)O)c3)c2cc1C1(C#N)CC1] | 1-(4-(((R)-1-(3-((S)-1,1-difluoro-2-hy- droxypropyl)phenyl)ethyl)amino)-7- methoxy-2-methylpyrido[2,3-d]pyrimidin- 6-yl)cyclopropane-1-carbonitrile | 454.3 |
| B217 | 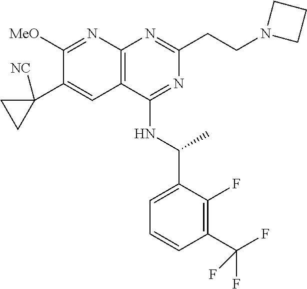 [SMILES: COc1nc2nc(CCN3CCC3)nc(N[C@H](C)c3cccc(C(F)(F)F)c3F)c2cc1C1(C#N)CC1] | (R)-1-(2-(2-(azetidin-1-yl)ethyl)-4-((1-(2- fluoro-3-(trifluoromethyl)phenyl)ethyl)amino)- 7-methoxypyrido[2,3-d]pyrimidin-6- yl)cyclopropane-1-carbonitrile | 497.4 |
| B218 | 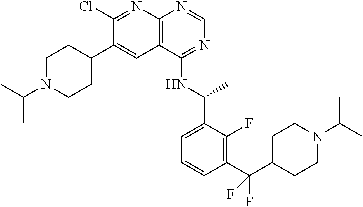 [SMILES: CC(C)N1CCC(c2cc3c(N[C@H](C)c4cccc(C(F)(F)C5CCN(C(C)C)CC5)c4F)ncnc3nc2Cl)CC1] | (R)-7-chloro-N-(1-(3-(difluoro(1- isopropylpiperidin-4-yl)methyl)-2- fluorophenyl)ethyl)-6-(1-isopropylpiperidin-4- yl)pyrido[2,3-d]pyrimidin-4-amine | 603.5 |
| B219 | 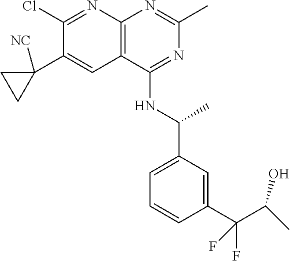 [SMILES: Cc1nc(N[C@H](C)c2cccc(C(F)(F)[C@@H](C)O)c2)c2cc(C3(C#N)CC3)c(Cl)nc2n1] | 1-(7-chloro-4-(((R)-1-(3-((R)-1,1-difluoro-2- hydroxypropyl)phenyl)ethyl)amino)-2- methylpyrido[2,3-d]pyrimidin-6- yl)cyclopropane-1-carbonitrile | 458.2 |
| B220 | 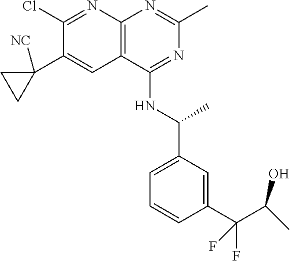 [SMILES: Cc1nc(N[C@H](C)c2cccc(C(F)(F)[C@H](C)O)c2)c2cc(C3(C#N)CC3)c(Cl)nc2n1] | 1-(7-chloro-4-(((R)-1-(3-((S)-1,1-difluoro-2- hydroxypropyl)phenyl)ethyl)amino)-2- methylpyrido[2,3-d]pyrimidin-6- yl)cyclopropane-1-carbonitrile | 458.2 |
| B221 | 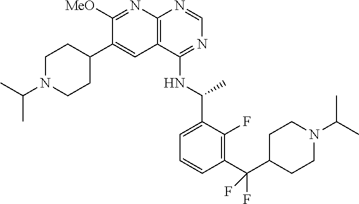 [SMILES: COc1nc2ncnc(N[C@H](C)c3cccc(C(F)(F)C4CCN(C(C)C)CC4)c3F)c2cc1C1CCN(C(C)C)CC1] | (R)-N-(1-(3-(difluoro(1-isopropylpiperidin-4- yl)methyl)-2-fluorophenyl)ethyl)-6-(1- isopropylpiperidin-4-yl)-7-methoxypyrido[2,3- d]pyrimidin-4-amine | 599.5 |
| B222 | 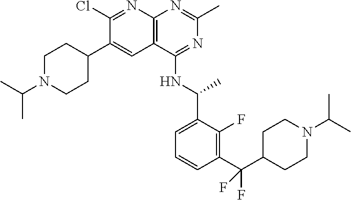 [SMILES: Cc1nc(N[C@H](C)c2cccc(C(F)(F)C3CCN(C(C)C)CC3)c2F)c2cc(C3CCN(C(C)C)CC3)c(Cl)nc2n1] | (R)-7-chloro-N-(1-(3-(difluoro(1- isopropylpiperidin-4-yl)methyl)-2-fluoro- phenyl)ethyl)-6-(1-isopropylpiperidin-4-yl)- 2-methylpyrido[2,3-d]pyrimidin-4-amine | 617.6 |
| B223 | 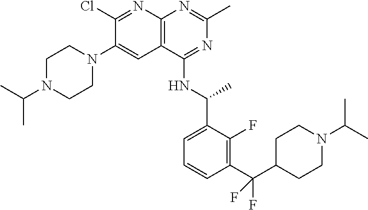 [SMILES: Cc1nc(N[C@H](C)c2cccc(C(F)(F)C3CCN(C(C)C)CC3)c2F)c2cc(N3CCN(C(C)C)CC3)c(Cl)nc2n1] | (R)-7-chloro-N-(1-(3-(difluoro(1- isopropylpiperidin-4-yl)methyl)-2-fluoro- phenyl)ethyl)-6-(4-isopropylpiperazin-1-yl)- 2-methylpyrido[2,3-d]pyrimidin-4-amine | 618.6 |
| B224 | 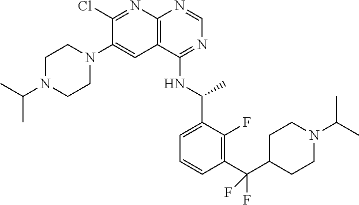 [SMILES: CC(C)N1CCC(C(F)(F)c2cccc([C@@H](C)Nc3ncnc4nc(Cl)c(N5CCN(C(C)C)CC5)cc34)c2F)CC1] | (R)-7-chloro-N-(1-(3-(difluoro(1- isopropylpiperidin-4-yl)methyl)-2- fluorophenyl)ethyl)-6-(4-isopropylpiperazin-1- yl)pyrido[2,3-d]pyrimidin-4-amine | 604.5 |
| B225 | 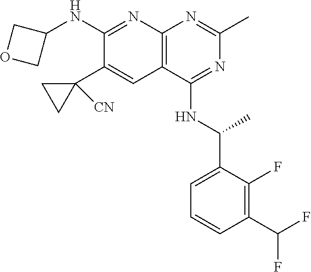 [SMILES: Cc1nc(N[C@H](C)c2cccc(C(F)F)c2F)c2cc(C3(C#N)CC3)c(NC3COC3)nc2n1] | (R)-1-(4-((1-(3-(difluoromethyl)-2- fluorophenyl)ethyl)amino)-2-methyl-7-(oxetan- 3-ylamino)pyrido[2,3-d]pyrimidin-6- yl)cyclopropane-1-carbonitrile | 469.25 |
| B226 | 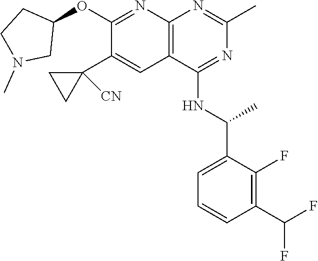 [SMILES: Cc1nc(N[C@H](C)c2cccc(C(F)F)c2F)c2cc(C3(C#N)CC3)c(O[C@@H]3CCN(C)C3)nc2n1] | 1-(4-(((R)-1-(3-(difluoromethyl)-2-fluoro- phenyl)ethyl)amino)-2-methyl-7-(((R)-1- methylpyrrolidin-3-yl)oxy)pyrido[2,3-d]pyrimi- din-6-yl)cyclopropane-1-carbonitrile | 497.4 |
| B227 | 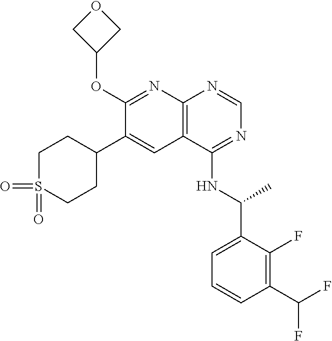 [SMILES: C[C@@H](Nc1ncnc2nc(OC3COC3)c(C3CCS(=O)(=O)CC3)cc12)c1cccc(C(F)F)c1F] | (R)-4-(4-((1-(3-(difluoromethyl)-2- fluorophenyl)ethyl)amino)-7-(oxetan-3- yloxy)pyrido[2,3-d]pyrimidin-6-yl)tetrahydro- 2H-thiopyran 1,1-dioxide | 523.8 |
| B228 | 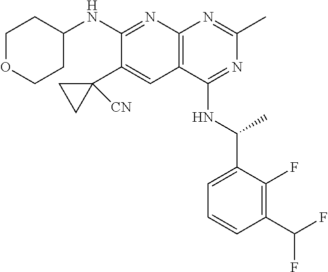 [SMILES: Cc1nc(N[C@H](C)c2cccc(C(F)F)c2F)c2cc(C3(C#N)CC3)c(NC3CCOCC3)nc2n1] | (R)-1-(4-((1-(3-(difluoromethyl)-2- fluorophenyl)ethyl)amino)-2-methyl-7- ((tetrahydro-2H-pyran-4-yl)amino)pyrido[2,3- d]pyrimidin-6-yl)cyclopropane-1-carbonitrile | 497.3 |
| B229 | 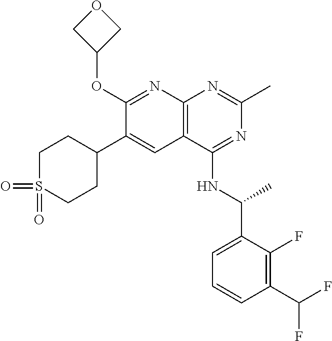 [SMILES: Cc1nc(N[C@H](C)c2cccc(C(F)F)c2F)c2cc(C3CCS(=O)(=O)CC3)c(OC3COC3)nc2n1] | (R)-4-(4-((1-(3-(difluoromethyl)-2-fluoro- phenyl)ethyl)amino)-2-methyl-7-(oxetan-3- yloxy)pyrido[2,3-d]pyrimidin-6-yl)tetrahydro- 2H-thiopyran 1,1-dioxide | 537.3 |
| B230 | 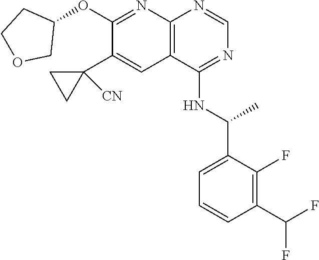 [SMILES: C[C@@H](Nc1ncnc2nc(O[C@H]3CCOC3)c(C3(C#N)CC3)cc12)c1cccc(C(F)F)c1F] | 1-(4-(((R)-1-(3-(difluoromethyl)-2-fluoro- phenyl)ethyl)amino)-7-(((S)-tetrahydrofuran- 3-yl)oxy)pyrido[2,3-d]pyrimidin-6- yl)cyclopropane-1-carbonitrile | 470.7 |
| B231 | 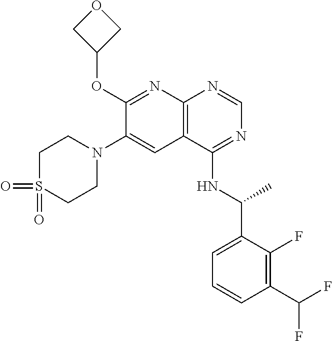 [SMILES: C[C@@H](Nc1ncnc2nc(OC3COC3)c(N3CCS(=O)(=O)CC3)cc12)c1cccc(C(F)F)c1F] | (R)-4-(4-((1-(3-(difluoromethyl)-2- fluorophenyl)ethyl)amino)-7-(oxetan-3- yloxy)pyrido[2,3-d]pyrimidin-6-yl)tetrahydro- 2H-thiopyran 1,1-dioxide | 524.3 |
| B232 | 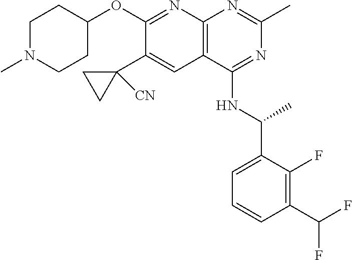 [SMILES: Cc1nc(N[C@H](C)c2cccc(C(F)F)c2F)c2cc(C3(C#N)CC3)c(OC3CCN(C)CC3)nc2n1] | (R)-1-(4-((1-(3-(difluoromethyl)-2- fluorophenyl)ethyl)amino)-2-methyl-7-((1- methylpiperidin-4-yl)oxy)pyrido[2,3- d]pyrimidin-6-yl)cyclopropane-1-carbonitrile | 511.3 |
| B233 | 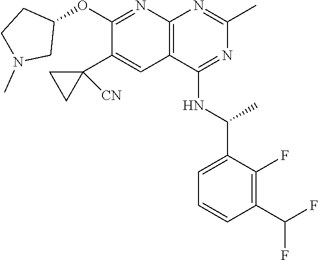 [SMILES: Cc1nc(N[C@H](C)c2cccc(C(F)F)c2F)c2cc(C3(C#N)CC3)c(O[C@H]3CCN(C)C3)nc2n1] | 1-(4-(((R)-1-(3-(difluoromethyl)-2-fluoro- phenyl)ethyl)amino)-2-methyl-7-(((S)-1- methylpyrrolidin-3-yl)oxy)pyrido[2,3-d]pyrimi- din-6-yl)cyclopropane-1-carbonitrile | 497.4 |
| B234 | 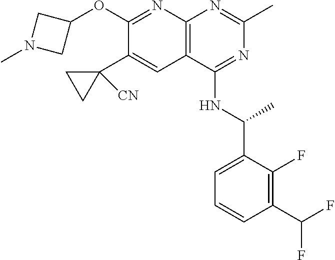 [SMILES: Cc1nc(N[C@H](C)c2cccc(C(F)F)c2F)c2cc(C3(C#N)CC3)c(OC3CN(C)C3)nc2n1] | (R)-1-(4-((1-(3-(difluoromethyl)-2- fluorophenyl)ethyl)amino)-2-methyl-7-((1- methylazetidin-3-yl)oxy)pyrido[2,3-d]pyrimi- din-6-yl)cyclopropane-1-carbonitrile | 483.3 |
| B235 | 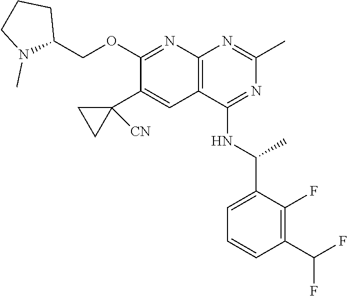 [SMILES: Cc1nc(N[C@H](C)c2cccc(C(F)F)c2F)c2cc(C3(C#N)CC3)c(OC[C@H]3CCCN3C)nc2n1] | 1-(4-(((R)-1-(3-(difluoromethyl)-2- fluorophenyl)ethyl)amino)-2-methyl-7-(((R)-1- methylpyrrolidin-2-yl)methoxy)pyrido[2,3- d]pyrimidin-6-yl)cyclopropane-1-carbonitrile | 511.4 |
| B236 | 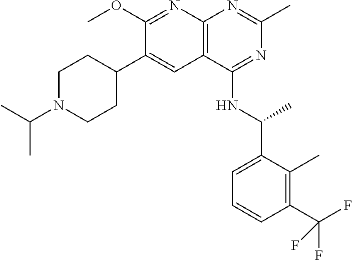 [SMILES: COc1nc2nc(C)nc(N[C@H](C)c3cccc(C(F)(F)F)c3C)c2cc1C1CCN(C(C)C)CC1] | (R)-6-(1-isopropylpiperidin-4-y1)-7-methoxy-2- methyl-N-(1-(2-methyl-3- (trifluoromethyl)phenyl)ethyl)pyrido[2,3- d]pyrimidin-4-amine | 502.3 |
| B237 | 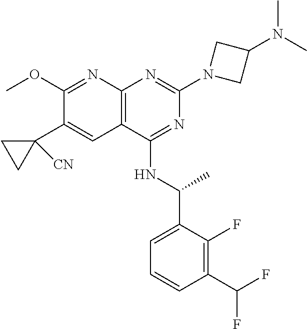 [SMILES: COc1nc2nc(N3CC(N(C)C)C3)nc(N[C@H](C)c3cccc(C(F)F)c3F)c2cc1C1(C#N)CC1] | (R)-1-(4-((1-(3-(difluoromethyl)-2- fluorophenyl)ethyl)amino)-2-(3- (dimethylamino)azetidin-1-yl)-7- methoxypyrido[2,3-d]pyrimidin-6- yl)cyclopropane-1-carbonitrile | 512.3 |
| B238 | 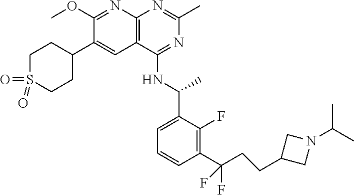 [SMILES: COc1nc2nc(C)nc(N[C@H](C)c3cccc(C(F)(F)CCC4CN(C(C)C)C4)c3F)c2cc1C1CCS(=O)(=O)CC1] | (R)-4-(4-((1-(3-(1,1-difluoro-3-(1- isopropylazetidin-3-yl)propyl)-2- fluorophenyl)ethyl)amino)-7-methoxy-2- methylpyrido[2,3-d]pyrimidin-6-yl)tetrahydro- 2H-thiopyran 1,1-dioxide | 620.4 |
| B239 | 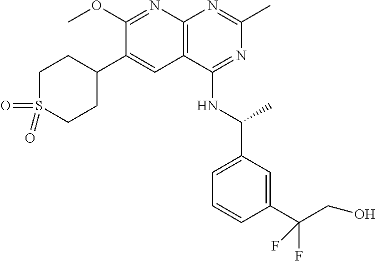 [SMILES: COc1nc2nc(C)nc(N[C@H](C)c3cccc(C(F)(F)CO)c3)c2cc1C1CCS(=O)(=O)CC1] | (R)-4-(4-((1-(3-(1,1-difluoro-2-hydroxy- ethyl)phenyl)ethyl)amino)-7-methoxy-2- methylpyrido[2,3-d]pyrimidin-6-yl)tetrahydro- 2H-thiopyran 1,1-dioxide | 507.6 |
| B240 | 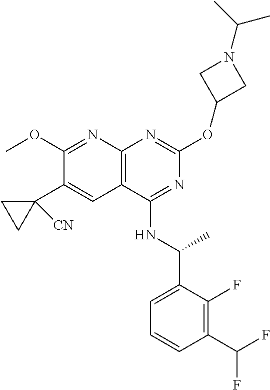 [SMILES: COc1nc2nc(OC3CN(C(C)C)C3)nc(N[C@H](C)c3cccc(C(F)F)c3F)c2cc1C1(C#N)CC1] | (R)-1-(4-((1-(3-(difluoromethyl)-2-fluoro- phenyl)ethyl)amino)-2-((1-isopropylazetidin- 3-yl)oxy)-7-methoxypyrido[2,3-d]pyrimidin-6- yl)cyclopropane-1-carbonitrile | 527.3 |
| B241 | 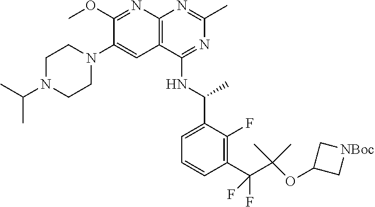 [SMILES: COc1nc2nc(C)nc(N[C@H](C)c3cccc(C(F)(F)C(C)(C)OC4CN(C(=O)OC(C)(C)C)C4)c3F)c2cc1N1CCN(C(C)C)CC1] | tert-butyl (R)-3-((1,1-difluoro-1-(2-fluoro-3- (1-((6-(4-isopropylpiperazin-1-yl)-7- methoxy-2-methylpyrido[2,3-d]pyrimidin-4- yl)amino)ethyl)phenyl)-2-methylpropan-2- yl)oxy)azetidine-1-carboxylate | 702.6 |
| B242 | 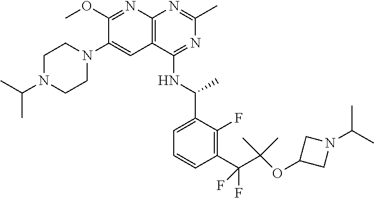 [SMILES: COc1nc2nc(C)nc(N[C@H](C)c3cccc(C(F)(F)C(C)(C)OC4CN(C(C)C)C4)c3F)c2cc1N1CCN(C(C)C)CC1] | (R)-N-(1-(3-(1,1-difluoro-2-((1- isopropylazetidin-3-yl)oxy)-2-methylpro- pyl)-2-fluorophenyl)ethyl)- 6-(4-isopropylpiperazin-1-yl)-7-methoxy-2- methylpyrido[2,3-d]pyrimidin-4-amine | 644.5 |
| B243 | 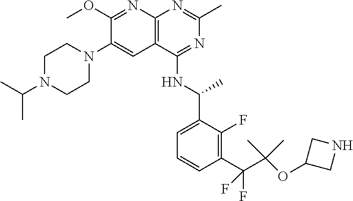 [SMILES: COc1nc2nc(C)nc(N[C@H](C)c3cccc(C(F)(F)C(C)(C)OC4CNC4)c3F)c2cc1N1CCN(C(C)C)CC1] | (R)-N-(1-(3-(2-(azetidin-3-yloxy)-1,1-difluoro- 2-methylpropyl)-2-fluorophenyl)ethyl)-6-(4- isopropylpiperazin-1-yl)-7-methoxy-2- methylpyrido[2,3-d]pyrimidin-4-amine | 602.5 |
| B244 | 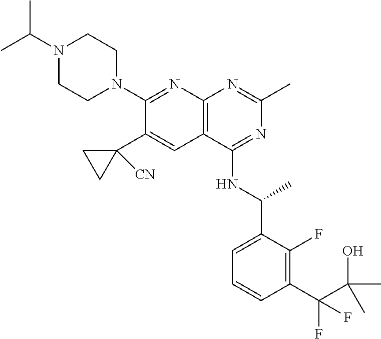 [SMILES: Cc1nc(N[C@H](C)c2cccc(C(F)(F)C(C)(C)O)c2F)c2cc(C3(C#N)CC3)c(N3CCN(C(C)C)CC3)nc2n1] | (R)-1-(4-((1-(3-(1,1-difluoro-2-hydroxy-2- methylpropyl)-2-fluorophenyl)ethyl)amino)- 7-(4-isopropylpiperazin-1-yl)-2-methyl- pyrido[2,3-d]pyrimidin-6-yl)cyclopropane-1- carbonitrile | 582.5 |
| B245 | 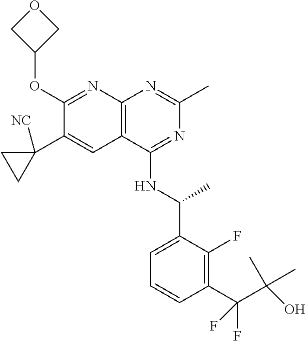 [SMILES: Cc1nc(N[C@H](C)c2cccc(C(F)(F)C(C)(C)O)c2F)c2cc(C3(C#N)CC3)c(OC3COC3)nc2n1] | (R)-1-(4-((1-(3-(1,1-difluoro-2-hydroxy-2- methylpropyl)-2-fluorophenyl)ethyl)amino)-2- methyl-7-(oxetan-3-yloxy)pyrido[2,3- d]pyrimidin-6-yl)cyclopropane-1-carbonitrile | 528.7 |
| B246 | 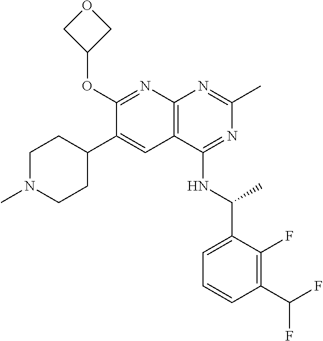 [SMILES: Cc1nc(N[C@H](C)c2cccc(C(F)F)c2F)c2cc(C3CCN(C)CC3)c(OC3COC3)nc2n1] | (R)-N-(1-(3-(difluoromethyl)-2- fluorophenyl)ethyl)-2-methyl-6-(1- methylpiperidin-4-yl)-7-(oxetan-3- yloxy)pyrido[2,3-d]pyrimidin-4-amine | 502.5 |
| B247 | 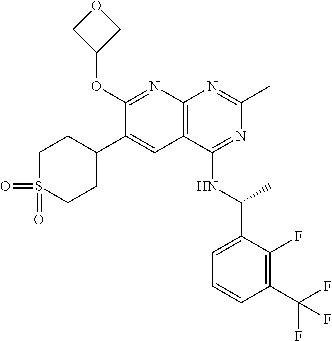 [SMILES: Cc1nc(N[C@H](C)c2cccc(C(F)(F)F)c2F)c2cc(C3CCS(=O)(=O)CC3)c(OC3COC3)nc2n1] | (R)-4-(4-((1-(2-fluoro-3-(trifluoro- methyl)phenyl)ethyl)amino)-2-methyl-7- (oxetan-3-yloxy)pyrido[2,3-d]pyrimidin-6- yl)tetrahydro-2H-thiopyran 1,1-dioxide | 555.8 |
| B248 | 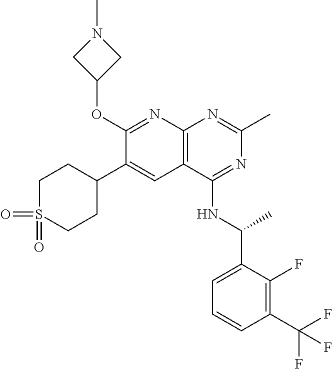 [SMILES: Cc1nc(N[C@H](C)c2cccc(C(F)(F)F)c2F)c2cc(C3CCS(=O)(=O)CC3)c(OC3CN(C)C3)nc2n1] | (R)-4-(4-((1-(2-fluoro-3-(trifluoro- methyl)phenyl)ethyl)amino)-2-methyl-7- ((1-methylazetidin-3-yl)oxy)pyrido[2,3- d]pyrimidin-6-yl)tetrahydro-2H-thiopyran 1,1- dioxide | 568.5 |
| B249 | 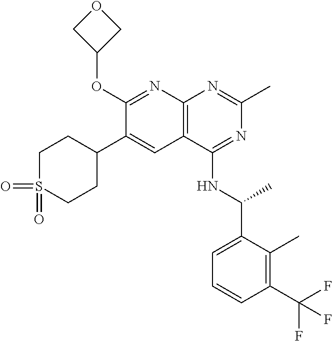 [SMILES: Cc1nc(N[C@H](C)c2cccc(C(F)(F)F)c2C)c2cc(C3CCS(=O)(=O)CC3)c(OC3COC3)nc2n1] | (R)-4-(2-methyl-4-((1-(2-methyl-3-(tri- fluoromethyl)phenyl)ethyl)amino)-7-(oxetan-3- yloxy)pyrido[2,3-d]pyrimidin-6-yl)tetrahydro- 2H-thiopyran 1,1-dioxide | 551.5 |
| B250 | 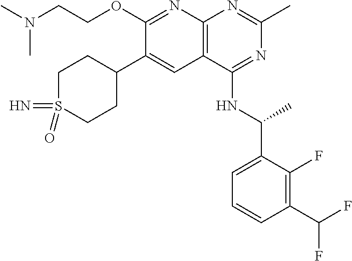 [SMILES: Cc1nc(N[C@H](C)c2cccc(C(F)F)c2F)c2cc(C3CCS(=N)(=O)CC3)c(OCCN(C)C)nc2n1] | (R)-4-(4-((1-(3-(difluoromethyl)-2- fluorophenyl)ethyl)amino)-7-(2- (dimethylamino)ethoxy)-2-methylpyrido[2,3- d]pyrimidin-6-yl)-1-iminohexahydro- 1l6-thiopyran1-oxide | 551.5 |
| B251 | 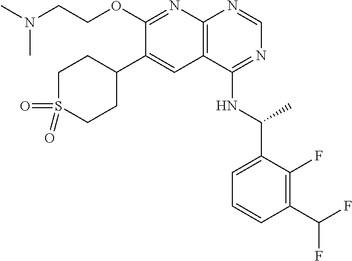 [SMILES: C[C@@H](Nc1ncnc2nc(OCCN(C)C)c(C3CCS(=O)(=O)CC3)cc12)c1cccc(C(F)F)c1F] | (R)-4-(4-((1-(3-(difluoromethyl)-2- fluorophenyl)ethyl)amino)-7-(2- (dimethylamino)ethoxy)pyrido[2,3-d]pyrimidin- 6-yl)tetrahydro-2H-thiopyran 1,1-dioxide | 538.5 |
| B252 | 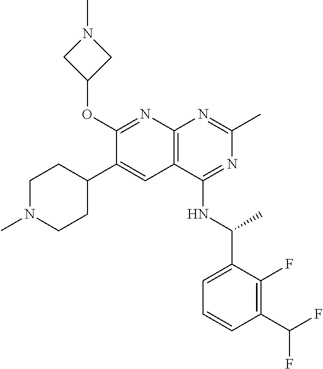 [SMILES: Cc1nc(N[C@H](C)c2cccc(C(F)F)c2F)c2cc(C3CCN(C)CC3)c(OC3CN(C)C3)nc2n1] | (R)-N-(1-(3-(difluoromethyl)-2- fluorophenyl)ethyl)-2-methyl-7-((1- methylazetidin-3-yl)oxy)-6-(1-methylpiperidin- 4-yl)pyrido[2,3-d]pyrimidin-4-amine | 515.5 |
| B253 | 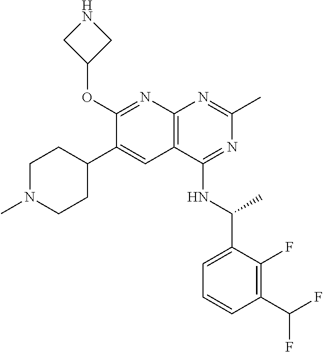 [SMILES: Cc1nc(N[C@H](C)c2cccc(C(F)F)c2F)c2cc(C3CCN(C)CC3)c(OC3CNC3)nc2n1] | (R)-7-(azetidin-3-yloxy)-N-(1-(3-(difluoro- methyl)-2-fluorophenyl)ethyl)-2-methyl-6-(1- methylpiperidin-4-yl)pyrido[2,3-d]pyrimidin-4- amine | 501.5 |
| B254 | 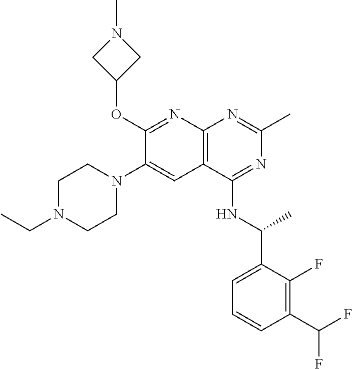 [SMILES: CCN1CCN(c2cc3c(N[C@H](C)c4cccc(C(F)F)c4F)nc(C)nc3nc2OC2CN(C)C2)CC1] | (R)-N-(1-(3-(difluoromethyl)-2-fluoro- phenyl)ethyl)-6-(4-ethylpiperazin-1-yl)-2- methyl-7-((1-methylazetidin-3-yl)oxy)pyri- do[2,3-d]pyrimidin-4-amine | 530.5 |
| B255 | 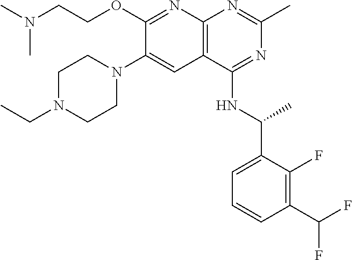 [SMILES: CCN1CCN(c2cc3c(N[C@H](C)c4cccc(C(F)F)c4F)nc(C)nc3nc2OCCN(C)C)CC1] | (R)-N-(1-(3-(difluoromethyl)-2-fluoro- phenyl)ethyl)-7-(2-(dimethylamino)ethoxy)- 6-(4-ethylpiperazin-1-yl)-2-methylpyrido[2,3- d]pyrimidin-4-amine | 532.5 |
| B256 | 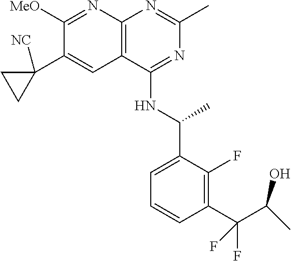 [SMILES: COc1nc2nc(C)nc(N[C@H](C)c3cccc(C(F)(F)[C@H](C)O)c3F)c2cc1C1(C#N)CC1] | 1-(4-(((R)-1-(3-((S)-1,1-difluoro-2- hydroxypropyl)-2-fluorophenyl)ethyl)amino)-7- methoxy-2-methylpyrido[2,3-d]pyrimidin-6- yl)cyclopropane-1-carbonitrile | 472.3 |
| B257 | 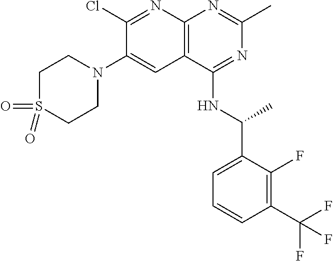 [SMILES: Cc1nc(N[C@H](C)c2cccc(C(F)(F)F)c2F)c2cc(N3CCS(=O)(=O)CC3)c(Cl)nc2n1] | (R)-4-(7-chloro-4-((1-(2-fluoro-3- (trifluoromethyl)phenyl)ethyl)amino)-2- methylpyrido[2,3-d]pyrimidin-6- yl)thiomorpholine 1,1-dioxide | 518.3 |
| B258 | 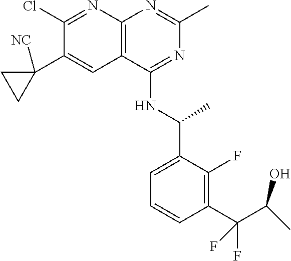 [SMILES: Cc1nc(N[C@H](C)c2cccc(C(F)(F)[C@H](C)O)c2F)c2cc(C3(C#N)CC3)c(Cl)nc2n1] | 1-(7-chloro-4-(((R)-1-(3-((S)-1,1-difluoro-2- hydroxypropyl)-2-fluorophenyl)ethyl)amino)- 2-methylpyrido[2,3-d]pyrimidin-6- yl)cyclopropane-1-carbonitrile | 476.3 |
| B259 | 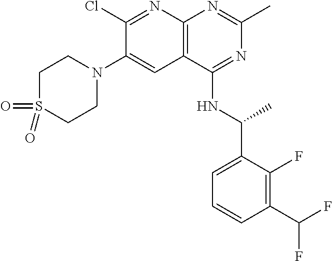 [SMILES: Cc1nc(N[C@H](C)c2cccc(C(F)F)c2F)c2cc(N3CCS(=O)(=O)CC3)c(Cl)nc2n1] | (R)-4-(7-chloro-4-((1-(3-(difluoromethyl)-2- fluorophenyl)ethyl)amino)-2-methylpyri- do[2,3-d]pyrimidin-6-yl)thiomorpholine 1,1-dioxide | 500.3 |
| B260 | 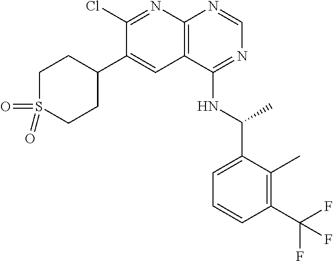 [SMILES: Cc1c([C@@H](C)Nc2ncnc3nc(Cl)c(C4CCS(=O)(=O)CC4)cc23)cccc1C(F)(F)F] | (R)-4-(7-chloro-4-((1-(2-methyl-3-(tri- fluoromethyl)phenyl)ethyl)amino)pyrido[2,3- d]pyrimidin-6-yl)tetrahydro-2H-thiopyran 1,1- dioxide | 499.3 |
| B261 | 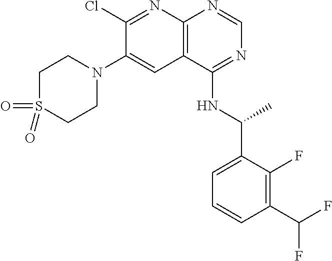 [SMILES: C[C@@H](Nc1ncnc2nc(Cl)c(N3CCS(=O)(=O)CC3)cc12)c1cccc(C(F)F)c1F] | (R)-4-(7-chloro-4-((1-(3-(difluoromethyl)-2- fluorophenyl)ethyl)amino)pyrido[2,3- d]pyrimidin-6-yl)thiomorpholine 1,1-dioxide | 486.2 |
| B262 | 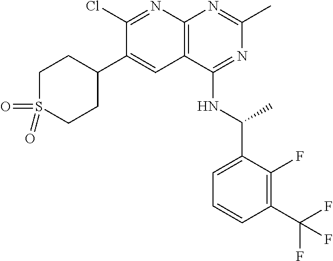 [SMILES: Cc1nc(N[C@H](C)c2cccc(C(F)(F)F)c2F)c2cc(C3CCS(=O)(=O)CC3)c(Cl)nc2n1] | (R)-4-(7-chloro-4-((1-(2-fluoro-3- (trifluoromethyl)phenyl)ethyl)amino)-2- methylpyrido[2,3-d]pyrimidin-6-yl)tetrahydro- 2H-thiopyran 1,1-dioxide | 517.3 |
| B263 | 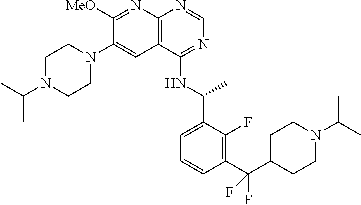 [SMILES: COc1nc2ncnc(N[C@H](C)c3cccc(C(F)(F)C4CCN(C(C)C)CC4)c3F)c2cc1N1CCN(C(C)C)CC1] | (R)-N-(1-(3-(difluoro(1-isopropylpiperidin-4- yl)methyl)-2-fluorophenyl)ethyl)-6-(4- isopropylpiperazin-1-yl)-7-methoxypyrido[2,3- d]pyrimidin-4-amine | 600.3 |
| B264 | 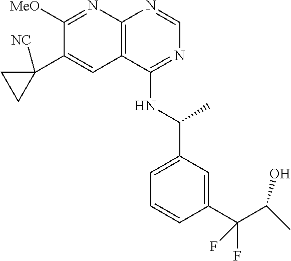 [SMILES: COc1nc2ncnc(N[C@H](C)c3cccc(C(F)(F)[C@@H](C)O)c3)c2cc1C1(C#N)CC1] | 1-(4-(((R)-1-(3-((R)-1,1-difluoro-2- hydroxypropyl)phenyl)ethyl)amino)-7- methoxypyrido[2,3-d]pyrimidin-6- yl)cyclopropane-1-carbonitrile | 440.3 |
| B265 | 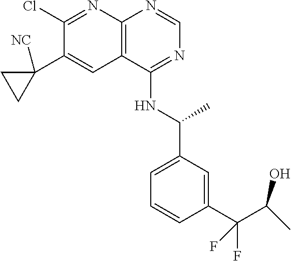 [SMILES: C[C@H](O)C(F)(F)c1cccc([C@@H](C)Nc2ncnc3nc(Cl)c(C4(C#N)CC4)cc23)c1] | 1-(7-chloro-4-(((R)-1-(3-((S)-1,1-difluoro-2- hydroxypropyl)phenyl)ethyl)amino)pyrido[2,3- d]pyrimidin-6-yl)cyclopropane-1-carbonitrile | 444. |
| B266 | 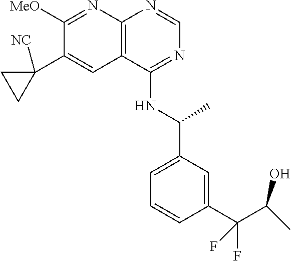 [SMILES: COc1nc2ncnc(N[C@H](C)c3cccc(C(F)(F)[C@H](C)O)c3)c2cc1C1(C#N)CC1] | 1-(4-(((R)-1-(3-((S)-1,1-difluoro-2- hydroxypropyl)phenyl)ethyl)amino)-7- methoxypyrido[2,3-d]pyrimidin-6- yl)cyclopropane-1-carbonitrile | 440.2 |
| B267 | 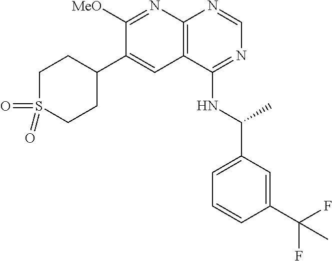 [SMILES: COc1nc2ncnc(N[C@H](C)c3cccc(C(C)(F)F)c3)c2cc1C1CCS(=O)(=O)CC1] | (R)-4-(4-((1-(3-(1,1-difluoroethyl)-2- fluorophenyl)ethyl)amino)-7-methoxypyrido[2,3- d]pyrimidin-6-yl)tetrahydro-2H-thiopyran 1,1- dioxide | 495.3 |
| B268 | 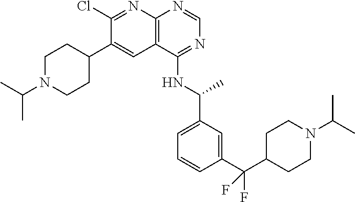 [SMILES: CC(C)N1CCC(c2cc3c(N[C@H](C)c4cccc(C(F)(F)C5CCN(C(C)C)CC5)c4)ncnc3nc2Cl)CC1] | (R)-7-chloro-N-(1-(3-(difluoro(1-iso- propylpiperidin-4-yl)methyl)phenyl)ethyl)-6-(1- isopropylpiperidin-4-yl)pyrido[2,3-d]pyrimidin- 4-amine | 585.3 |
| B269 | 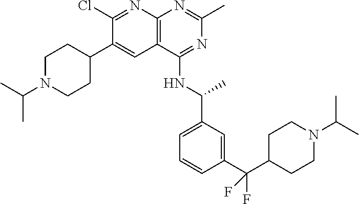 [SMILES: Cc1nc(N[C@H](C)c2cccc(C(F)(F)C3CCN(C(C)C)CC3)c2)c2cc(C3CCN(C(C)C)CC3)c(Cl)nc2n1] | (R)-7-chloro-N-(1-(3-(difluoro(1-iso- propylpiperidin-4-yl)methyl)phenyl)ethyl)-6-(1- isopropylpiperidin-4-yl)-2-methylpyrido[2,3- d]pyrimidin-4-amine | 599.6 |
| B270 | 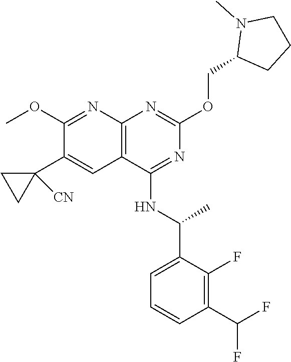 [SMILES: COc1nc2nc(OC[C@H]3CCCN3C)nc(N[C@H](C)c3cccc(C(F)F)c3F)c2cc1C1(C#N)CC1] | 1-(4-(((R)-1-(3-(difluoromethyl)-2- fluorophenyl)ethyl)amino)-7-methoxy-2-(((R)-1- methylpyrrolidin-2-yl)methoxy)pyrido[2,3- d]pyrimidin-6-yl)cyclopropane-1-carbonitrile | 527.5 |
| B271 | 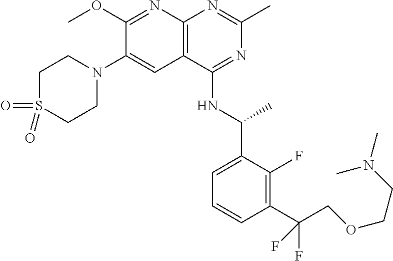 [SMILES: COc1nc2nc(C)nc(N[C@H](C)c3cccc(C(F)(F)COCCN(C)C)c3F)c2cc1N1CCS(=O)(=O)CC1] | (R)-4-(4-((1-(3-(2-(2-(dimethylamino)ethoxy)- 1,1-difluoroethyl)-2-fluorophenyl)ethyl)amino)- 7-methoxy-2-methylpyrido[2,3-d]pyrimidin-6- yl)thiomorpholine 1,1-dioxide | 596.5 |
| B272 | 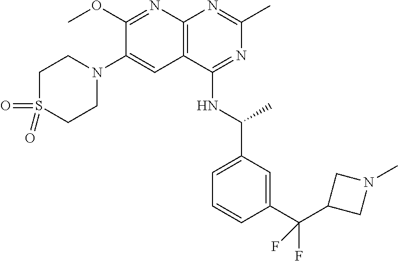 [SMILES: COc1nc2nc(C)nc(N[C@H](C)c3cccc(C(F)(F)C4CN(C)C4)c3)c2cc1N1CCS(=O)(=O)CC1] | (R)-4-(4-((1-(3-(difluoro(1-methylazetidin-3- yl)methyl)phenyl)ethyl)amino)-7-methoxy-2- methylpyrido[2,3-d]pyrimidin-6- yl)thiomorpholine 1,1-dioxide | 546.4 |
| B273 | 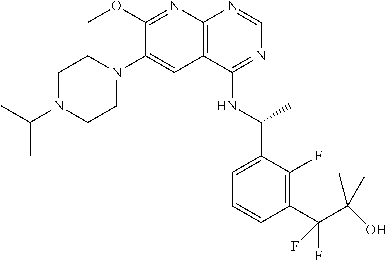 [SMILES: COc1nc2ncnc(N[C@H](C)c3cccc(C(F)(F)C(C)(C)O)c3F)c2cc1N1CCN(C(C)C)CC1] | (R)-1,1-difluoro-1-(2-fluoro-3-(1-((6-(4- isopropylpiperazin-1-yl)-7-methoxypyrido[2,3- d]pyrimidin-4-yl)amino)ethyl)phenyl)-2- methylpropan-2-ol | 533.5 |
| B274 | 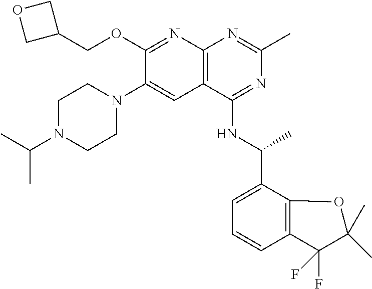 [SMILES: Cc1nc(N[C@H](C)c2cccc3c2OC(C)(C)C3(F)F)c2cc(N3CCN(C(C)C)CC3)c(OCC3COC3)nc2n1] | (R)-N-(1-(3,3-difluoro-2,2-dimethyl-2,3- dihydrobenzofuran-7-yl)ethyl)-6-(4- isopropylpiperazin-1-yl)-2-methyl-7-(oxetan-3- ylmethoxy)pyrido[2,3-d]pyrimidin-4-amine | 583.6 |
| B275 | 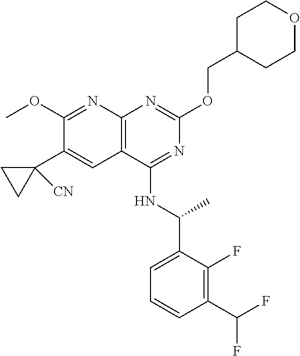 [SMILES: COc1nc2nc(OCC3CCOCC3)nc(N[C@H](C)c3cccc(C(F)F)c3F)c2cc1C1(C#N)CC1] | (R)-1-(4-((1-(3-(difluoromethyl)-2- fluorophenyl)ethyl)amino)-7-methoxy-2- ((tetrahydro-2H-pyran-4-yl)methoxy)pyrido[2,3- d]pyrimidin-6-yl)cyclopropane-1-carbonitrile | 528.8 |
| B276 | 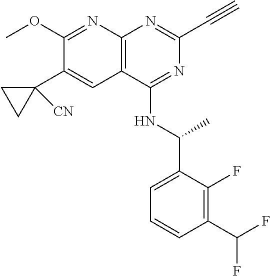 [SMILES: C#Cc1nc(N[C@H](C)c2cccc(C(F)F)c2F)c2cc(C3(C#N)CC3)c(OC)nc2n1] | (R)-1-(4-((1-(3-(difluoromethyl)-2- fluorophenyl)ethyl)amino)-2-ethynyl-7- methoxypyrido[2,3-d]pyrimidin-6- yl)cyclopropane-1-carbonitrile | 438.3 |
| B277 | 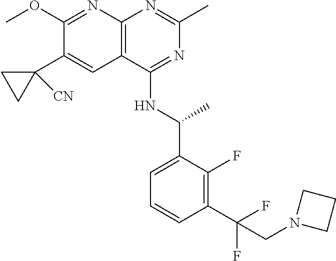 [SMILES: COc1nc2nc(C)nc(N[C@H](C)c3cccc(C(F)(F)CN4CCC4)c3F)c2cc1C1(C#N)CC1] | (R)-1-(4-((1-(3-(2-(azetidin-1-yl)-1,1- difluoroethyl)-2-fluorophenyl)ethyl)amino)-7- methoxy-2-methylpyrido[2,3-d]pyrimidin-6- yl)cyclopropane-1-carbonitrile | 497.4 |
| B278 | 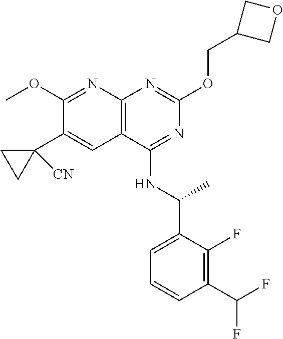 [SMILES: COc1nc2nc(OCC3COC3)nc(N[C@H](C)c3cccc(C(F)F)c3F)c2cc1C1(C#N)CC1] | (R)-1-(4-((1-(3-(difluoromethyl)-2- fluorophenyl)ethyl)amino)-7-methoxy-2-(oxetan- 3-ylmethoxy)pyrido[2,3-d]pyrimidin-6- yl)cyclopropane-1-carbonitrile | 500.4 |
| B279 | 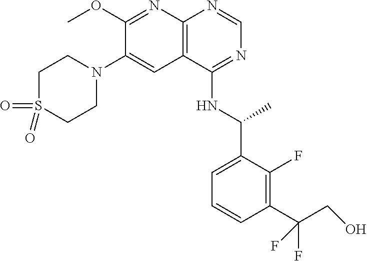 [SMILES: COc1nc2ncnc(N[C@H](C)c3cccc(C(F)(F)CO)c3F)c2cc1N1CCS(=O)(=O)CC1] | (R)-4-(4-((1-(3-(1,1-difluoro-2-hydroxyethyl)- 2-fluorophenyl)ethyl)amino)-7-methoxy- pyrido[2,3-d]pyrimidin-6-yl)thiomorpholine 1,1-dioxide | 512.3 |
| B280 | 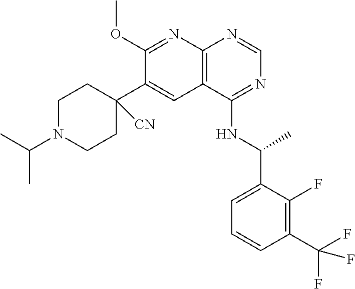 [SMILES: COc1nc2ncnc(N[C@H](C)c3cccc(C(F)(F)F)c3F)c2cc1C1(C#N)CCN(C(C)C)CC1] | (R)-4-(4-((1-(2-fluoro-3- (trifluoromethyl)phenyl)ethyl)amino)-7- methoxypyrido[2,3-d]pyrimidin-6-yl)-1- isopropylpiperidine-4-carbonitrile | 517.4 |
| B281 | 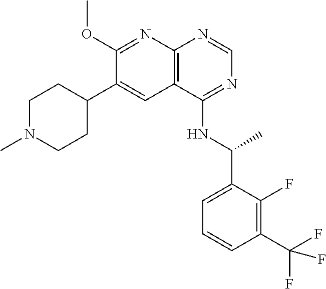 [SMILES: COc1nc2ncnc(N[C@H](C)c3cccc(C(F)(F)F)c3F)c2cc1C1CCN(C)CC1] | (R)-N-(1-(2-fluoro-3- (trifluoromethyl)phenyl)ethyl)-7-methoxy-6-(1- methylpiperidin-4-yl)pyrido[2,3-d]pyrimidin-4- amine | 464.3 |
| B282 | 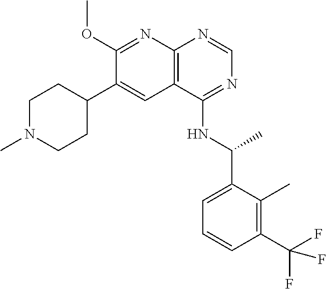 [SMILES: COc1nc2ncnc(N[C@H](C)c3cccc(C(F)(F)F)c3C)c2cc1C1CCN(C)CC1] | (R)-7-methoxy-N-(1-(2-methyl-3- (trifluoromethyl)phenyl)ethyl)-6-(1- methylpiperidin-4-yl)pyrido[2,3-d]pyrimidin-4- amine | 460.3 |
| B283 | 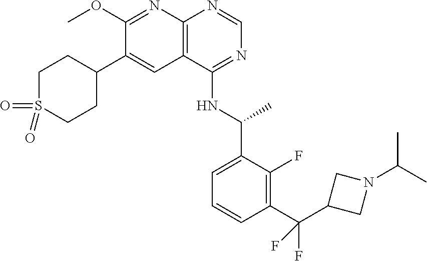 [SMILES: COc1nc2ncnc(N[C@H](C)c3cccc(C(F)(F)C4CN(C(C)C)C4)c3F)c2cc1C1CCS(=O)(=O)CC1] | (R)-4-(4-((1-(3-(difluoro(1-isopropylazetidin-3- yl)methyl)-2-fluorophenyl)ethyl)amino)-7- methoxypyrido[2,3-d]pyrimidin-6-yl)tetrahydro- 2H-thiopyran 1,1-dioxide | 578.4 |
| B284 | 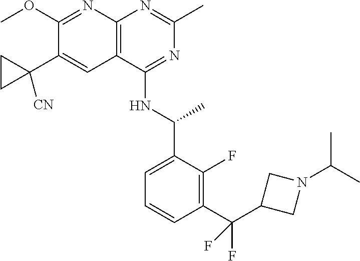 [SMILES: COc1nc2nc(C)nc(N[C@H](C)c3cccc(C(F)(F)C4CN(C(C)C)C4)c3F)c2cc1C1(C#N)CC1] | (R)-1-(4-((1-(3-(difluoro(1-isopropylazetidin-3- yl)methyl)-2-fluorophenyl)ethyl)amino)-7- methoxy-2-methylpyrido[2,3-d]pyrimidin-6- yl)cyclopropane-1-carbonitrile | 525.4 |
| B285 | 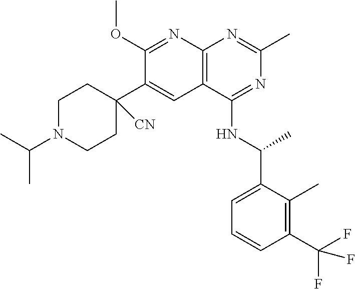 [SMILES: COc1nc2nc(C)nc(N[C@H](C)c3cccc(C(F)(F)F)c3C)c2cc1C1(C#N)CCN(C(C)C)CC1] | (R)-1-isopropyl-4-(7-methoxy-2-methyl-4-((1- (2-methyl-3- (trifluoromethyl)phenyl)ethyl)amino)pyrido [2,3-d]pyrimidin-6-yl)piperidine-4-carbonitrile | 527.4 |
| B286 | 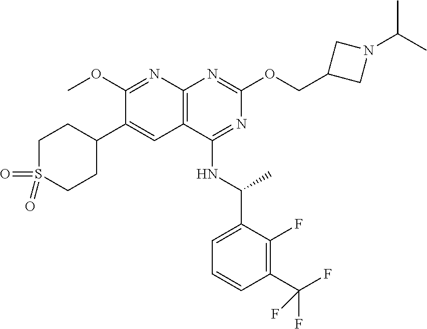 [SMILES: COc1nc2nc(OCC3CN(C(C)C)C3)nc(N[C@H](C)c3cccc(C(F)(F)F)c3F)c2cc1C1CCS(=O)(=O)CC1] | (R)-4-(4-((1-(2-fluoro-3- (trifluoromethyl)phenyl)ethyl)amino)-2-((1- isopropylazetidin-3-yl)methoxy)-7- methoxypyrido[2,3-d]pyrimidin-6-yl)tetrahydro- 2H-thiopyran 1,1-dioxide | 626.4 |
| B287 | 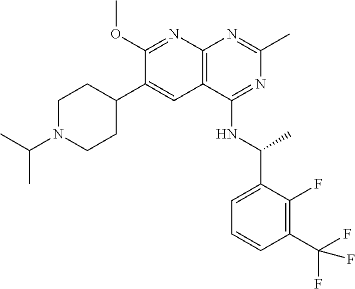 [SMILES: COc1nc2nc(C)nc(N[C@H](C)c3cccc(C(F)(F)F)c3F)c2cc1C1CCN(C(C)C)CC1] | (R)-N-(1-(2-fluoro-3- (trifluoromethyl)phenyl)ethyl)-6-(1- isopropylpiperidin-4-yl)-7-methoxy-2- methylpyrido[2,3-d]pyrimidin-4-amine | 506.5 |
| B288 | 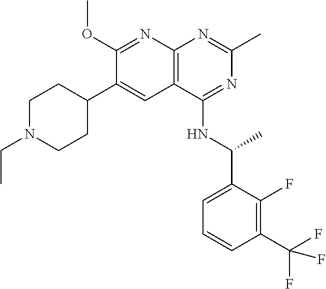 [SMILES: CCN1CCC(c2cc3c(N[C@H](C)c4cccc(C(F)(F)F)c4F)nc(C)nc3nc2OC)CC1] | (R)-6-(1-ethylpiperidin-4-yl)-N-(1-(2-fluoro-3- (trifluoromethyl)phenyl)ethyl)-7-methoxy-2- methylpyrido[2,3-d]pyrimidin-4-amine | 492.4 |
| B289 | 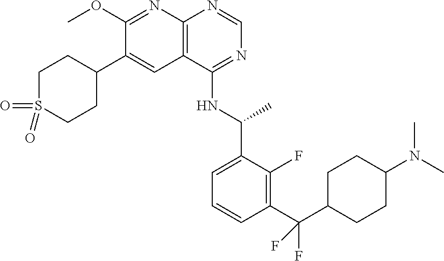 [SMILES: COc1nc2ncnc(N[C@H](C)c3cccc(C(F)(F)C4CCC(N(C)C)CC4)c3F)c2cc1C1CCS(=O)(=O)CC1] | (R)-4-(4-((1-(3-((4- (dimethylamino)cyclohexyl)difluoromethyl)-2- fluorophenyl)ethyl)amino)-7-methoxypyrido [2,3-d]pyrimidin-6-yl)tetrahydro-2H-thiopyran 1,1-dioxide | 606 |
| B290 | 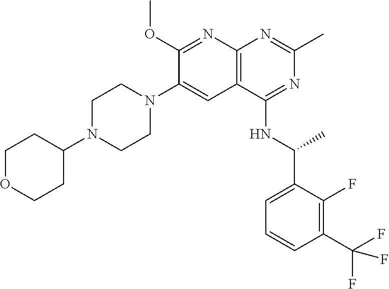 [SMILES: COc1nc2nc(C)nc(N[C@H](C)c3cccc(C(F)(F)F)c3F)c2cc1N1CCN(C2CCOCC2)CC1] | (R)-N-(1-(2-fluoro-3-(trifluoro- methyl)phenyl)ethyl)-7-methoxy-2- methyl-6-(4-(tetrahydro-2H-pyran-4- yl)piperazin-1-yl)pyrido[2,3- d]pyrimidin-4-amine | 549.4 |
| B291 | 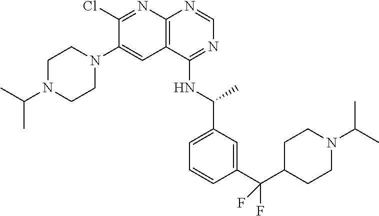 [SMILES: CC(C)N1CCC(C(F)(F)c2cccc([C@@H](C)Nc3ncnc4nc(Cl)c(N5CCN(C(C)C)CC5)cc34)c2)CC1] | (R)-7-chloro-N-(1-(3-(difluoro(1- isopropylpiperidin-4-yl)methyl)phe- nyl)ethyl)-6-(4-isopropylpiperazin- 1-yl)pyrido[2,3-d]pyrimidin-4- amine | 586.5 |
| B292 | 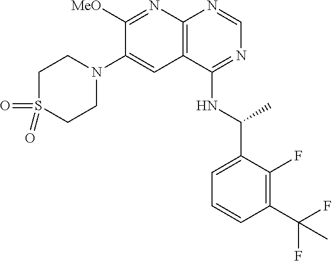 [SMILES: COc1nc2ncnc(N[C@H](C)c3cccc(C(C)(F)F)c3F)c2cc1N1CCS(=O)(=O)CC1] | (R)-4-(4-((1-(3-(1,1-difluoroethyl)-2-fluoro- phenyl)ethyl)amino)-7-methoxypyrido[2,3- d]pyrimidin-6-yl)thiomorpholine 1,1-dioxide | 496.4 |
| B293 | 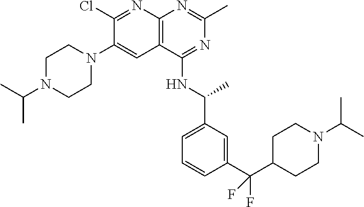 [SMILES: Cc1nc(N[C@H](C)c2cccc(C(F)(F)C3CCN(C(C)C)CC3)c2)c2cc(N3CCN(C(C)C)CC3)c(Cl)nc2n1] | (R)-7-chloro-N-(1-(3-(difluoro(1-iso- propylpiperidin-4-yl)methyl)phenyl)ethyl)-6-(4- isopropylpiperazin-1-yl)-2-methylpyrido[2,3- d]pyrimidin-4-amine | 600.6 |
| B294 | 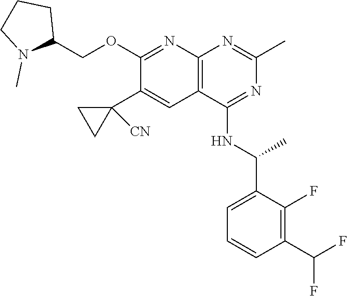 | (R)-1-(4-((1-(3-(difluoromethyl)-2- fluorophenyl)ethyl)amino)-7-((5-(di- methylamino)pentyl)oxy)-2-methylpyrido[2,3- d]pyrimidin-6-yl)cyclopropane-1-carbonitrile | 511.4 |
| B295 | 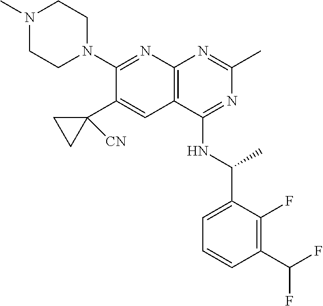 [SMILES: Cc1nc(N[C@H](C)c2cccc(C(F)F)c2F)c2cc(C3(C#N)CC3)c(N3CCN(C)CC3)nc2n1] | (R)-1-(4-((1-(3-(difluoromethyl)-2- fluorophenyl)ethyl)amino)-2-methyl-7-(4- methylpiperazin-1-yl)pyrido[2,3-d]pyrimidin- 6-yl)cyclopropane-1-carbonitrile | 496.3 |
| B296 | 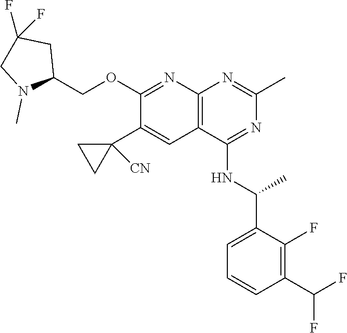 [SMILES: Cc1nc(N[C@H](C)c2cccc(C(F)F)c2F)c2cc(C3(C#N)CC3)c(OC[C@@H]3CC(F)(F)CN3C)nc2n1] | 1-(7-(((S)-4,4-difluoro-1-methylpyrrolidin-2- yl)methoxy)-4-(((R)-1-(3-(difluoromethyl)-2- fluorophenyl)ethyl)amino)-2-methylpyrido[2,3- d]pyrimidin-6-yl)cyclopropane-1-carbonitrile | 547.2 |
| B297 | 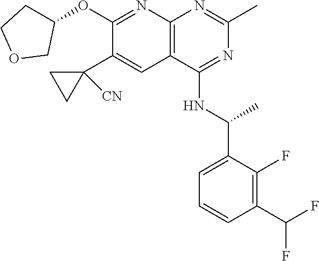 [SMILES: Cc1nc(N[C@H](C)c2cccc(C(F)F)c2F)c2cc(C3(C#N)CC3)c(O[C@H]3CCOC3)nc2n1] | 1-(4-(((R)-1-(3-(difluoromethyl)-2- fluorophenyl)ethyl)amino)-2-methyl-7-(((S)- tetrahydrofuran-3-yl)oxy)pyrido[2,3- d]pyrimidin-6-yl)cyclopropane-1-carbonitrile | 483.3 |
| B298 | 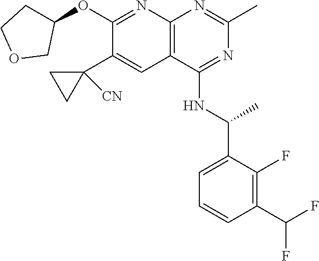 [SMILES: Cc1nc(N[C@H](C)c2cccc(C(F)F)c2F)c2cc(C3(C#N)CC3)c(O[C@@H]3CCOC3)nc2n1] | 1-(4-(((R)-1-(3-(difluoromethyl)-2- fluorophenyl)ethyl)amino)-2-methyl-7-(((R)- tetrahydrofuran-3-yl)oxy)pyrido[2,3- d]pyrimidin-6-yl)cyclopropane-1-carbonitrile | 483.3 |
| B299 | 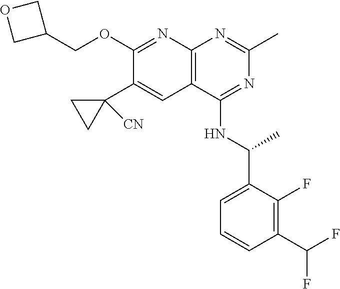 [SMILES: Cc1nc(N[C@H](C)c2cccc(C(F)F)c2F)c2cc(C3(C#N)CC3)c(OCC3COC3)nc2n1] | (R)-1-(4-((1-(3-(difluoromethyl)-2-fluoro- phenyl)ethyl)amino)-2-methyl-7-(oxetan-3- ylmethoxy)pyrido[2,3-d]pyrimidin-6- yl)cyclopropane-1-carbonitrile | 484.7 |
| B300 | 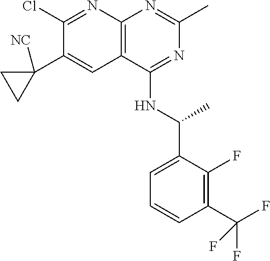 [SMILES: Cc1nc(N[C@H](C)c2cccc(C(F)(F)F)c2F)c2cc(C3(C#N)CC3)c(Cl)nc2n1] | (R)-1-(7-chloro-4-((1-(2-fluoro-3- (trifluoromethyl)phenyl)ethyl)amino)-2- methylpyrido[2,3-d]pyrimidin-6- yl)cyclopropane-1-carbonitrile | 450.5 |
| B301 | 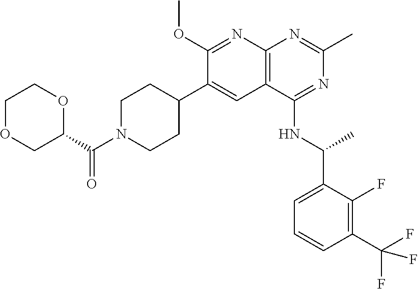 [SMILES: COc1nc2nc(C)nc(N[C@H](C)c3cccc(C(F)(F)F)c3F)c2cc1C1CCN(C(=O)[C@@H]2COCCO2)CC1] | ((S)-1,4-dioxan-2-y1)(4-(4-(((R)-1-(2-fluoro-3- (trifluoromethyl)phenyl)ethyl)amino)-7- methoxy-2-methylpyrido[2,3-d]pyrimidin-6- yl)piperidin-1-yl)methanone | 578.8 |
| B302 | 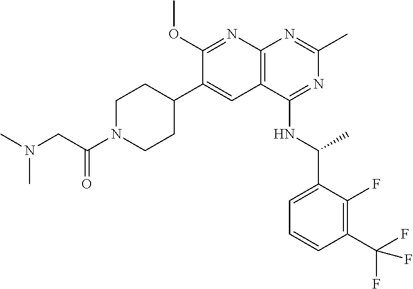 [SMILES: COc1nc2nc(C)nc(N[C@H](C)c3cccc(C(F)(F)F)c3F)c2cc1C1CCN(C(=O)CN(C)C)CC1] | (R)-2-(dimethylamino)-1-(4-(4-((1-(2-fluoro- 3-(trifluoromethyl)phenyl)ethyl)amino)- 7-methoxy-2-methylpyrido[2,3-d]pyrimidin- 6-yl)piperidin-1-yl)ethan-1-one | 549.4 |
| B303 | 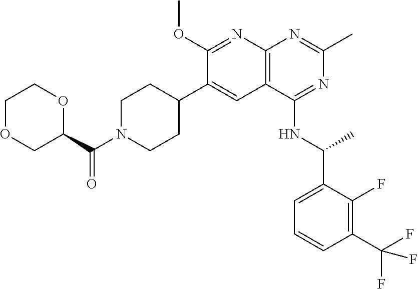 [SMILES: COc1nc2nc(C)nc(N[C@H](C)c3cccc(C(F)(F)F)c3F)c2cc1C1CCN(C(=O)[C@H]2COCCO2)CC1] | ((R)-1,4-dioxan-2-y1)(4-(4-(((R)-1-(2-fluoro- 3-(trifluoromethyl)phenyl)ethyl)amino)-7- methoxy-2-methylpyrido[2,3-d]pyrimidin- 6-yl)piperidin-1-yl)methanone | 578.4 |
| B304 | 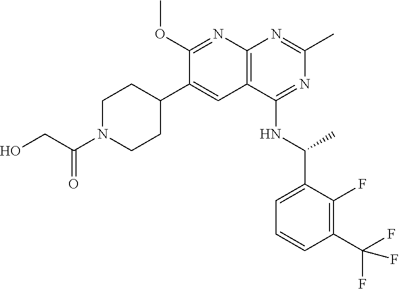 [SMILES: COc1nc2nc(C)nc(N[C@H](C)c3cccc(C(F)(F)F)c3F)c2cc1C1CCN(C(=O)CO)CC1] | (R)-1-(4-(4-((1-(2-fluoro-3-(trifluoro- methyl)phenyl)ethyl)amino)-7-methoxy- 2-methylpyrido[2,3-d]pyrimidin-6-yl)piperidin- 1-yl)-2-hydroxyethan-1-one | 522.8 |
| B305 | 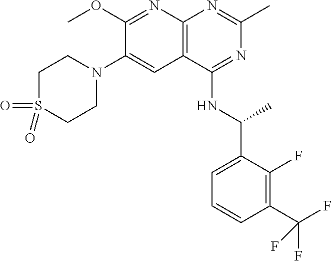 [SMILES: COc1nc2nc(C)nc(N[C@H](C)c3cccc(C(F)(F)F)c3F)c2cc1N1CCS(=O)(=O)CC1] | (R)-4-(4-((1-(2-fluoro-3-(tri- fluoromethyl)phenyl)ethyl)amino)-7-methoxy- 2-methylpyrido[2,3-d]pyrimidin-6- yl)thiomorpholine 1,1-dioxide | 514.3 |
| B306 | 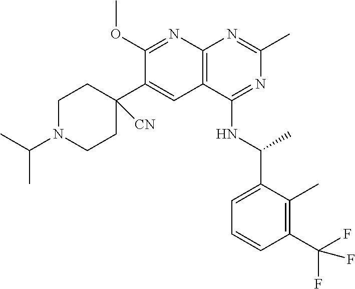 [SMILES: COc1nc2nc(C)nc(N[C@H](C)c3cccc(C(F)(F)F)c3C)c2cc1C1(C#N)CCN(C(C)C)CC1] | (R)-4-(4-((1-(2-fluoro-3-(tri- fluoromethyl)phenyl)ethyl)amino)-7-methoxy- 2-methylpyrido[2,3-d]pyrimidin-6-yl)-1- isopropylpiperidine-4-carbonitrile | 531.4 |
| B307 | 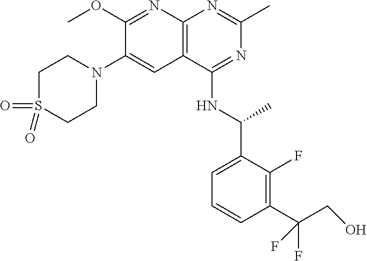 [SMILES: COc1nc2nc(C)nc(N[C@H](C)c3cccc(C(F)(F)CO)c3F)c2cc1N1CCS(=O)(=O)CC1] | (R)-4-(4-((1-(3-(1,1-difluoro-2-hydroxyethyl)- 2-fluorophenyl)ethyl)amino)-7-methoxy- 2-methylpyrido[2,3-d]pyrimidin-6- yl)thiomorpholine 1,1-dioxide | 526.7 |
| B308 | 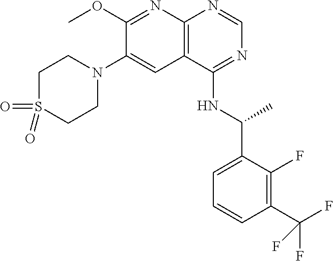 [SMILES: COc1nc2ncnc(N[C@H](C)c3cccc(C(F)(F)F)c3F)c2cc1N1CCS(=O)(=O)CC1] | (R)-4-(4-((1-(2-fluoro-3- (trifluoromethyl)phenyl)ethyl)amino)-7- methoxypyrido[2,3-d]pyrimidin-6- yl)thiomorpholine 1,1-dioxide | 500.6 |
| B309 | 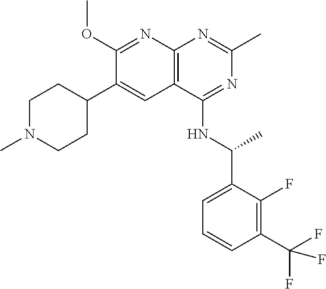 [SMILES: COc1nc2nc(C)nc(N[C@H](C)c3cccc(C(F)(F)F)c3F)c2cc1C1CCN(C)CC1] | (R)-N-(1-(2-fluoro-3- (trifluoromethyl)phenyl)ethyl)-7-methoxy-2- methyl-6-(1-methylpiperidin-4-yl)pyrido[2,3- d]pyrimidin-4-amine | 478.3 |
| B310 | 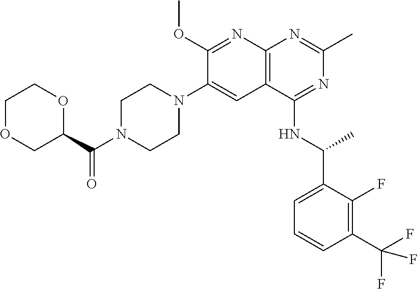 [SMILES: COc1nc2nc(C)nc(N[C@H](C)c3cccc(C(F)(F)F)c3F)c2cc1N1CCN(C(=O)[C@H]2COCCO2)CC1] | ((R)-1,4-dioxan-2-yl)(4-(4-(((R)-1-(2-fluoro- 3-(trifluoromethyl)phenyl)ethyl)amino)-7- methoxy-2-methylpyrido[2,3-d]pyrimidin- 6-yl)piperazin-1-yl)methanone | 579.4 |
| B311 | 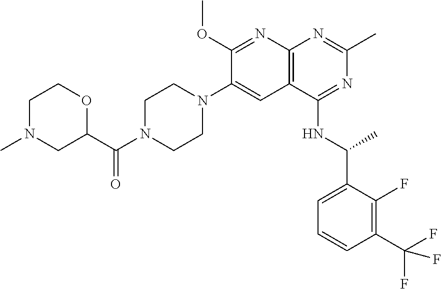 [SMILES: COc1nc2nc(C)nc(N[C@H](C)c3cccc(C(F)(F)F)c3F)c2cc1N1CCN(C(=O)C2CN(C)CCO2)CC1] | (4-(4-(((R)-1-(2-fluoro-3-(trifluoro- methyl)phenyl)ethyl)amino)-7-methoxy- 2-methylpyrido[2,3-d]pyrimidin-6-yl)piperazin- 1-yl)(4-methylmorpholin-2-yl)methanone | 592.4 |
| B312 | 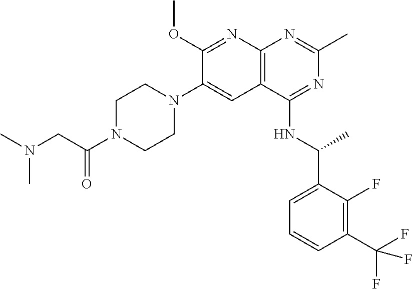 [SMILES: COc1nc2nc(C)nc(N[C@H](C)c3cccc(C(F)(F)F)c3F)c2cc1N1CCN(C(=O)CN(C)C)CC1] | (R)-2-(dimethylamino)-1-(4-(4-((1-(2-fluoro-3- (trifluoromethyl)phenyl)ethyl)amino)-7- methoxy-2-methylpyrido[2,3-d]pyrimidin- 6-yl)piperazin-1-yl)ethan-1-one | 550.4 |
| B313 | 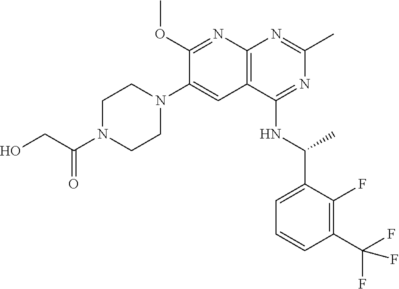 [SMILES: COc1nc2nc(C)nc(N[C@H](C)c3cccc(C(F)(F)F)c3F)c2cc1N1CCN(C(=O)CO)CC1] | (R)-1-(4-(4-((1-(2-fluoro-3-(trifluoro- methyl)phenyl)ethyl)amino)-7-methoxy- 2-methylpyrido[2,3-d]pyrimidin-6- yl)piperazin-1-yl)-2-hydroxyethan-1-one | 523.4 |
| B314 | 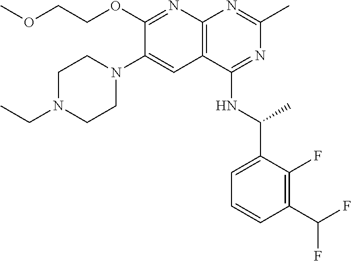 [SMILES: CCN1CCN(c2cc3c(N[C@H](C)c4cccc(C(F)F)c4F)nc(C)nc3nc2OCCOC)CC1] | (R)-N-(1-(3-(difluoromethyl)-2-fluoro- phenyl)ethyl)-6-(4-ethylpiperazin-1-yl)-7-(2- methoxyethoxy)-2-methylpyrido[2,3- d]pyrimidin-4-amine | 519.5 |
| B315 | 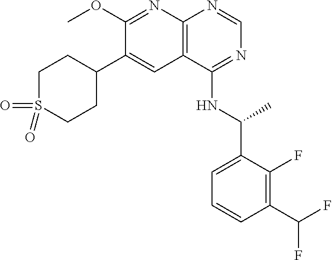 [SMILES: COc1nc2ncnc(N[C@H](C)c3cccc(C(F)F)c3F)c2cc1C1CCS(=O)(=O)CC1] | (R)-4-(4-((1-(3-(difluoromethyl)-2- fluorophenyl)ethyl)amino)-7-methoxy- pyrido[2,3-d]pyrimidin-6-yl)tetrahydro- 2H-thiopyran 1,1-dioxide | 481.3 |
| B316 | 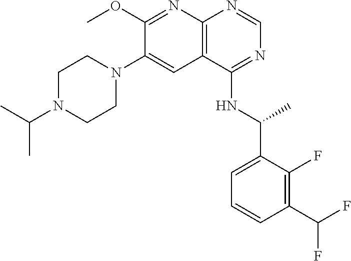 [SMILES: COc1nc2ncnc(N[C@H](C)c3cccc(C(F)F)c3F)c2cc1N1CCN(C(C)C)CC1] | (R)-N-(1-(3-(difluoromethyl)-2-fluoro- phenyl)ethyl)-6-(4-isopropylpiperazin-1-yl)- 7-methoxypyrido[2,3-d]pyrimidin-4-amine | 475.4 |
| B317 | 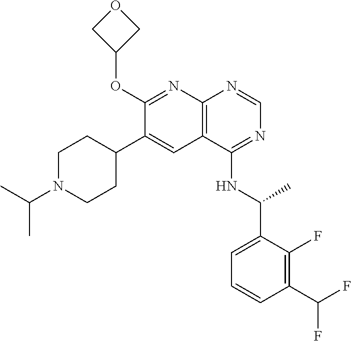 [SMILES: CC(C)N1CCC(c2cc3c(N[C@H](C)c4cccc(C(F)F)c4F)ncnc3nc2OC2COC2)CC1] | (R)-N-(1-(3-(difluoromethyl)-2- fluorophenyl)ethyl)-6-(1-isopropyl- piperidin-4-yl)-7-(oxetan- 3-yloxy)pyrido[2,3-d]pyrimidin-4-amine | 516.5 |
| B318 | 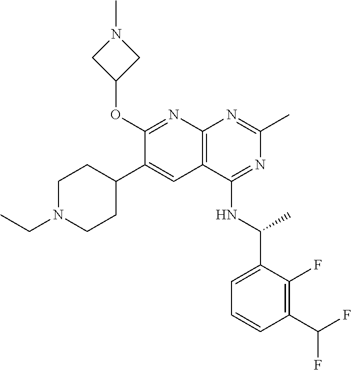 [SMILES: CCN1CCC(c2cc3c(N[C@H](C)c4cccc(C(F)F)c4F)nc(C)nc3nc2OC2CN(C)C2)CC1] | (R)-4-(4-((1-(3-(difluoromethyl)-2-fluoro- phenyl)ethyl)amino)-7-methoxypyrido[2,3- d]pyrimidin-6-yl)tetrahydro-2H-thiopyran 1,1-dioxide | 529.5 |
| B319 | 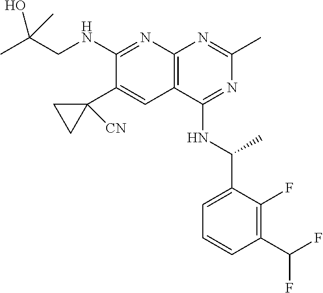 [SMILES: Cc1nc(N[C@H](C)c2cccc(C(F)F)c2F)c2cc(C3(C#N)CC3)c(NCC(C)(C)O)nc2n1] | (R)-1-(4-((1-(3-(difluoromethyl)-2- fluorophenyl)ethyl)amino)-7-((2-hydroxy-2- methylpropyl)amino)-2-methylpyrido[2,3- d]pyrimidin-6-yl)cyclopropane-1-carbonitrile | 485.3 |
| B320 | 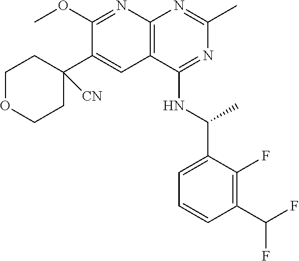 [SMILES: COc1nc2nc(C)nc(N[C@H](C)c3cccc(C(F)F)c3F)c2cc1C1(C#N)CCOCC1] | (R)-4-(4-((1-(3-(difluoromethyl)-2- fluorophenyl)ethyl)amino)-7-methoxy-2- methylpyrido[2,3-d]pyrimidin-6-yl)tetrahydro- 2H-pyran-4-carbonitrile | 472.6 |
| B321 | 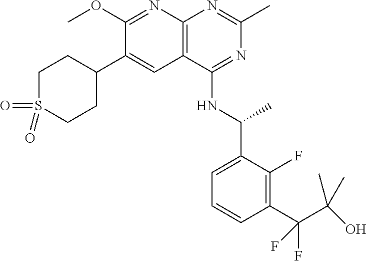 [SMILES: COc1nc2nc(C)nc(N[C@H](C)c3cccc(C(F)(F)C(C)(C)O)c3F)c2cc1C1CCS(=O)(=O)CC1] | (R)-4-(4-((1-(3-(1,1-difluoro-2-hydroxy-2- methylpropyl)-2-fluorophenyl)ethyl)amino)-7- methoxy-2-methylpyrido[2,3-d]pyrimidin-6- yl)tetrahydro-2H-thiopyran 1,1-dioxide | 553.3 |
| B322 | 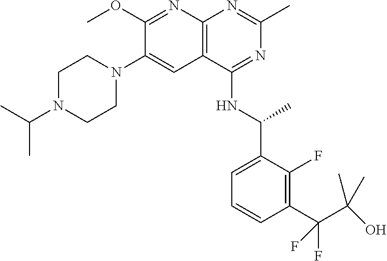 [SMILES: COc1nc2nc(C)nc(N[C@H](C)c3cccc(C(F)(F)C(C)(C)O)c3F)c2cc1N1CCN(C(C)C)CC1] | (R)-1,1-difluoro-1-(2-fluoro-3-(1-((6-(4- isopropylpiperazin-1-yl)-7-methoxy-2- methylpyrido[2,3-d]pyrimidin-4- yl)amino)ethyl)phenyl)-2-methylpropan-2-ol | 547.5 |
| B323 | 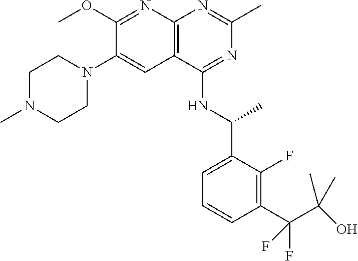 [SMILES: COc1nc2nc(C)nc(N[C@H](C)c3cccc(C(F)(F)C(C)(C)O)c3F)c2cc1N1CCN(C)CC1] | (R)-1,1-difluoro-1-(2-fluoro-3-(1-((7-methoxy- 2-methyl-6-(4-methylpiperazin-1-yl)pyri- do[2,3-d]pyrimidin-4-yl)amino)ethyl)phenyl)- 2-methylpropan-2-ol | 519.4 |
| B324 | 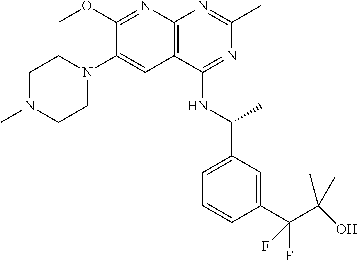 [SMILES: COc1nc2nc(C)nc(N[C@H](C)c3cccc(C(F)(F)C(C)(C)O)c3)c2cc1N1CCN(C)CC1] | (R)-1,1-difluoro-1-(3-(1-((7-methoxy- 2-methyl-6-(4-methylpiperazin- 1-yl)pyrido[2,3-d]pyrimidin-4- yl)amino)ethyl)phenyl)-2-methylpropan- 2-ol | 501.5 |
| B325 | 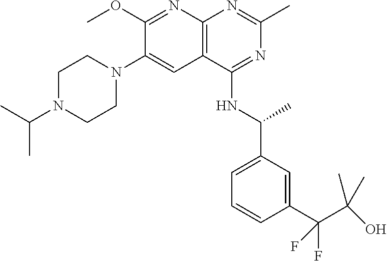 [SMILES: COc1nc2nc(C)nc(N[C@H](C)c3cccc(C(F)(F)C(C)(C)O)c3)c2cc1N1CCN(C(C)C)CC1] | (R)-1,1-difluoro-1-(3-(1-((6-(4- isopropylpiperazin-1-yl)-7-methoxy- 2-methylpyrido[2,3-d]pyrimidin- 4-yl)amino)ethyl)phenyl)-2- methylpropan-2-ol | 529.5 |
| B326 | 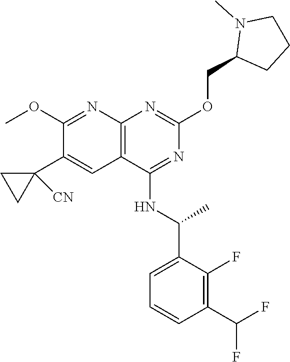 [SMILES: COc1nc2nc(OC[C@@H]3CCCN3C)nc(N[C@H](C)c3cccc(C(F)F)c3F)c2cc1C1(C#N)CC1] | 1-(4-(((R)-1-(3-(difluoromethyl)-2-fluoro- phenyl)ethyl)amino)-7-methoxy-2-(((S)-1- methylpyrrolidin-2-yl)methoxy)pyrido[2,3- d]pyrimidin-6-yl)cyclopropane-1-carbonitrile | 527.5 |
| B327 | 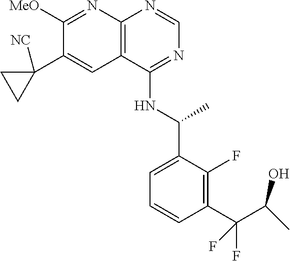 [SMILES: COc1nc2ncnc(N[C@H](C)c3cccc(C(F)(F)[C@H](C)O)c3F)c2cc1C1(C#N)CC1] | 1-(4-(((R)-1-(3-((S)-1,1-difluoro-2- hydroxypropyl)-2-fluorophenyl)ethyl)amino)- 7-methoxypyrido[2,3-d]pyrimidin-6- yl)cyclopropane-1-carbonitrile | 458.7 |
| B328 | 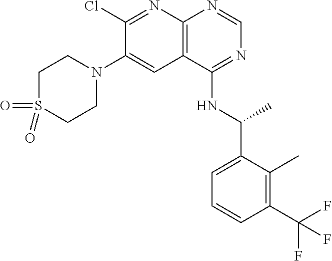 [SMILES: Cc1c([C@@H](C)Nc2ncnc3nc(Cl)c(N4CCS(=O)(=O)CC4)cc23)cccc1C(F)(F)F] | (R)-4-(7-chloro-4-((1-(2-methyl-3-(tri- fluoromethyl)phenyl)ethyl)amino)pyrido[2,3- d]pyrimidin-6-yl)thiomorpholine 1,1-dioxide | 500.2 |
| B329 | 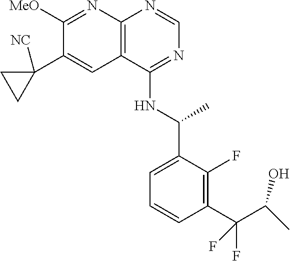 [SMILES: COc1nc2ncnc(N[C@H](C)c3cccc(C(F)(F)[C@@H](C)O)c3F)c2cc1C1(C#N)CC1] | 1-(4-(((R)-1-(3-((R)-1,1-difluoro-2- hydroxypropyl)-2-fluorophenyl)ethyl)amino)- 7-methoxypyrido[2,3-d]pyrimidin-6- yl)cyclopropane-1-carbonitrile | 458.7 |
| B330 | 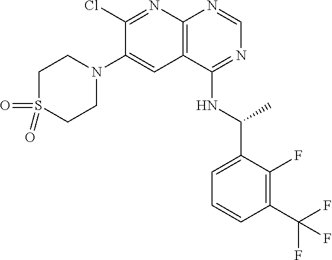 [SMILES: C[C@@H](Nc1ncnc2nc(Cl)c(N3CCS(=O)(=O)CC3)cc12)c1cccc(C(F)(F)F)c1F] | (R)-4-(7-chloro-4-((1-(2-fluoro-3-(tri- fluoromethyl)phenyl)ethyl)amino)pyrido[2,3- d]pyrimidin-6-yl)thiomorpholine 1,1-dioxide | 504.2 |
| B331 | 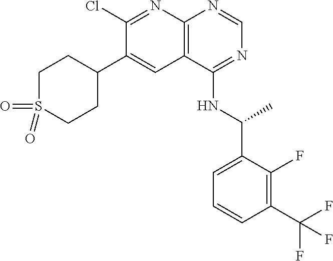 [SMILES: C[C@@H](Nc1ncnc2nc(Cl)c(C3CCS(=O)(=O)CC3)cc12)c1cccc(C(F)(F)F)c1F] | (R)-4-(7-chloro-4-((1-(2-fluoro-3-(tri- fluoromethyl)phenyl)ethyl)amino)pyrido[2,3- d]pyrimidin-6-yl)tetrahydro-2H-thiopyran 1,1- dioxide | 503.2 |
| B332 | 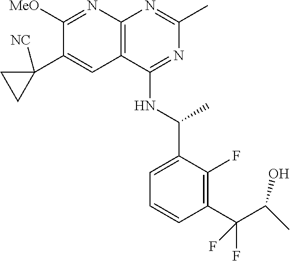 [SMILES: COc1nc2nc(C)nc(N[C@H](C)c3cccc(C(F)(F)[C@@H](C)O)c3F)c2cc1C1(C#N)CC1] | 1-(4-(((R)-1-(3-((R)-1,1-difluoro-2-hy- droxypropyl)-2-fluorophenyl)ethyl)amino)-7- methoxy-2-methylpyrido[2,3-d]pyrimidin-6- yl)cyclopropane-1-carbonitrile | 472.3 |
| B333 | 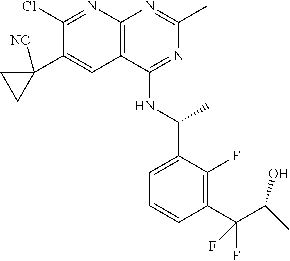 [SMILES: Cc1nc(N[C@H](C)c2cccc(C(F)(F)[C@@H](C)O)c2F)c2cc(C3(C#N)CC3)c(Cl)nc2n1] | 1-(7-chloro-4-(((R)-1-(3-((R)-1,1-difluoro-2- hydroxypropyl)-2-fluorophenyl)ethyl)amino)- 2-methylpyrido[2,3-d]pyrimidin- 16-yl)cyclopropane--carbonitrile | 476.3 |
| B334 | 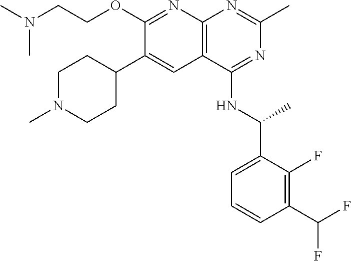 [SMILES: Cc1nc(N[C@H](C)c2cccc(C(F)F)c2F)c2cc(C3CCN(C)CC3)c(OCCN(C)C)nc2n1] | (R)-N-(1-(3-(difluoromethyl)-2-fluoro- phenyl)ethyl)-7-(2-(dimethylamino)ethoxy)- 2-methyl-6-(1-methylpiperidin-4-yl)pyrido[2,3- d]pyrimidin-4-amine | 517.5 |
| B335 | 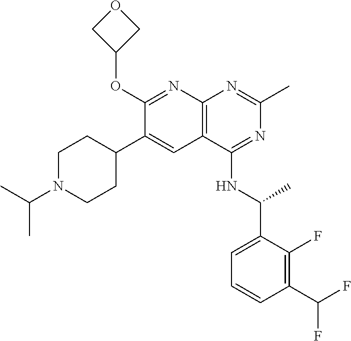 [SMILES: Cc1nc(N[C@H](C)c2cccc(C(F)F)c2F)c2cc(C3CCN(C(C)C)CC3)c(OC3COC3)nc2n1] | (R)-N-(1-(3-(difluoromethyl)-2- fluorophenyl)ethyl)-6-(1-isopropylpiperidin- 4-yl)-2-methyl-7-(oxetan-3-yloxy)pyrido[2,3- d]pyrimidin-4-amine | 530.5 |
| B336 | 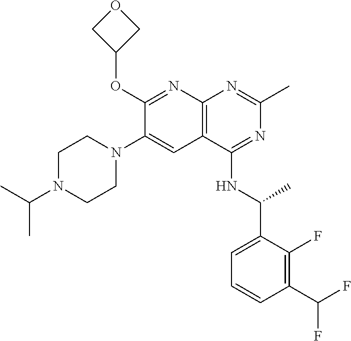 [SMILES: Cc1nc(N[C@H](C)c2cccc(C(F)F)c2F)c2cc(N3CCN(C(C)C)CC3)c(OC3COC3)nc2n1] | (R)-N-(1-(3-(difluoromethyl)-2- fluorophenyl)ethyl)-6-(4-isopropylpiperazin- 1-yl)-2-methyl-7-(oxetan-3-yloxy)pyrido[2,3- d]pyrimidin-4-amine | 531.5 |
| B337 | 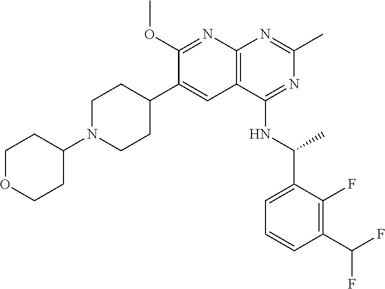 [SMILES: COc1nc2nc(C)nc(N[C@H](C)c3cccc(C(F)F)c3F)c2cc1C1CCN(C2CCOCC2)CC1] | (R)-N-(1-(3-(difluoromethyl)-2- fluorophenyl)ethyl)-7-methoxy-2-methyl-6-(1- (tetrahydro-2H-pyran-4-yl)piperidin-4- yl)pyrido[2,3-d]pyrimidin-4-amine | 530.5 |
| B338 | 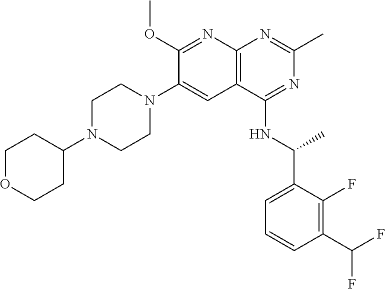 [SMILES: COc1nc2nc(C)nc(N[C@H](C)c3cccc(C(F)F)c3F)c2cc1N1CCN(C2CCOCC2)CC1] | (R)-N-(1-(3-(difluoromethyl)-2- fluorophenyl)ethyl)-7-methoxy-2-methyl-6-(4- (tetrahydro-2H-pyran-4-y1)piperazin-1- yl)pyrido[2,3-d]pyrimidin-4-amine | 531.5 |
| B339 | 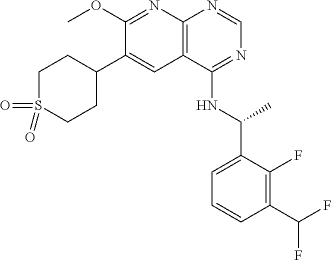 [SMILES: COc1nc2ncnc(N[C@H](C)c3cccc(C(F)F)c3F)c2cc1C1CCS(=O)(=O)CC1] | (R)-4-(4-((1-(3-(difluoromethyl)-2-fluoro- phenyl)ethyl)amino)-7-methoxypyrido[2,3- d]pyrimidin-6-yl)thiomorpholine 1,1-dioxide | 482.3 |
| B340 | 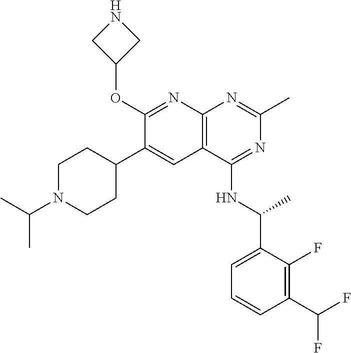 [SMILES: Cc1nc(N[C@H](C)c2cccc(C(F)F)c2F)c2cc(C3CCN(C(C)C)CC3)c(OC3CNC3)nc2n1] | (R)-7-(azetidin-3-yloxy)-N-(1- (3-(difluoromethyl)-2-fluoro- phenyl)ethyl)-6-(1-isopropylpiperidin-4- yl)-2-methylpyrido[2,3-d]pyrimidin-4-amine | 529.5 |
| B341 | 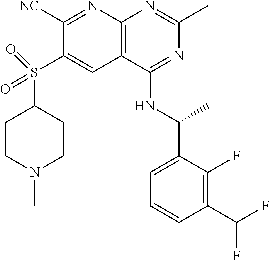 [SMILES: Cc1nc(N[C@H](C)c2cccc(C(F)F)c2F)c2cc(S(=O)(=O)C3CCN(C)CC3)c(C#N)nc2n1] | (R)-4-((1-(3-(difluoromethyl)-2- fluorophenyl)ethyl)amino)-2-methyl-6-((1- methylpiperidin-4-yl)sulfonyl)pyrido[2,3- d]pyrimidine-7-carbonitrile | 519.4 |
| B342 | 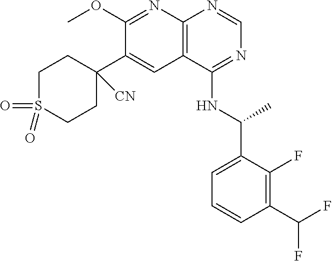 [SMILES: COc1nc2ncnc(N[C@H](C)c3cccc(C(F)F)c3F)c2cc1C1(C#N)CCS(=O)(=O)CC1] | (R)-4-(4-((1-(3-(difluoromethyl)-2-fluoro- phenyl)ethyl)amino)-7-methoxypyrido[2,3- d]pyrimidin-6-yl)tetrahydro-2H-thiopyran-4- carbonitrile 1,1-dioxide | 506.3 |
| B343 | 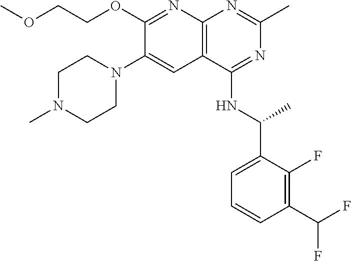 [SMILES: COCCOc1nc2nc(C)nc(N[C@H](C)c3cccc(C(F)F)c3F)c2cc1N1CCN(C)CC1] | (R)-N-(1-(3-(difluoromethyl)-2- fluorophenyl)ethyl)-7-(2-methoxyethoxy)-2- methyl-6-(4-methylpiperazin-1-yl)pyrido[2,3- d]pyrimidin-4-amine | 505.5 |
| B344 | 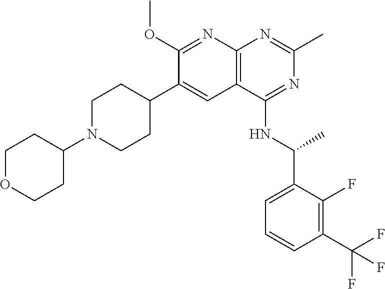 [SMILES: COc1nc2nc(C)nc(N[C@H](C)c3cccc(C(F)(F)F)c3F)c2cc1C1CCN(C2CCOCC2)CC1] | (R)-N-(1-(2-fluoro-3-(trifluoro- methyl)phenyl)ethyl)-7-methoxy-2- methyl-6-(1-(tetrahydro-2H-pyran-4- yl)piperidin-4-yl)pyrido[2,3-d]pyrimidin- 4-amine | 548.5 |
| B345 | 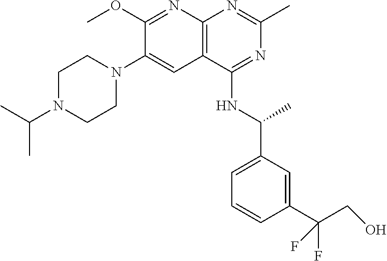 [SMILES: COc1nc2nc(C)nc(N[C@H](C)c3cccc(C(F)(F)CO)c3)c2cc1N1CCN(C(C)C)CC1] | (R)-2,2-difluoro-2-(3-(1-((6-(4- isopropylpiperazin-1-yl)- 7-methoxy-2-methylpyrido[2,3-d]pyrimidin- 4-yl)amino)ethyl)phenyl)ethan-1-ol | 501.4 |
| B346 | 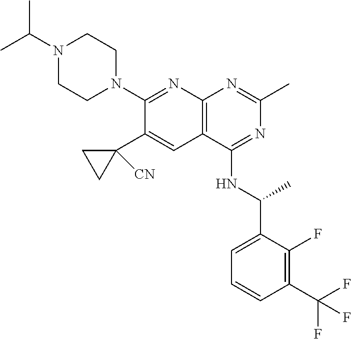 [SMILES: Cc1nc(N[C@H](C)c2cccc(C(F)(F)F)c2F)c2cc(C3(C#N)CC3)c(N3CCN(C(C)C)CC3)nc2n1] | (R)-1-(4-((1-(2-fluoro-3- (trifluoromethyl)phenyl)ethyl)amino)-7-(4- isopropylpiperazin-1-yl)-2-methylpyrido[2,3- d]pyrimidin-6-yl)cyclopropane-1-carbonitrile | 542.4 |
| B347 | 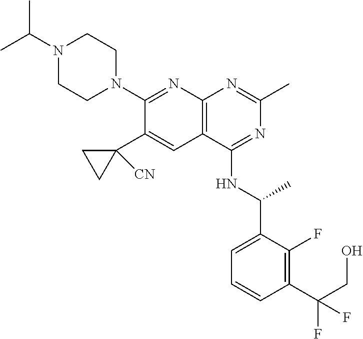 [SMILES: Cc1nc(N[C@H](C)c2cccc(C(F)(F)CO)c2F)c2cc(C3(C#N)CC3)c(N3CCN(C(C)C)CC3)nc2n1] | (R)-1-(4-((1-(3-(1,1-difluoro-2-hydroxyethyl)- 2-fluorophenyl)ethyl)amino)-7-(4- isopropylpiperazin-1-yl)-2-methylpyrido[2,3- d]pyrimidin-6-yl)cyclopropane-1-carbonitrile | 554.4 |
| B348 | 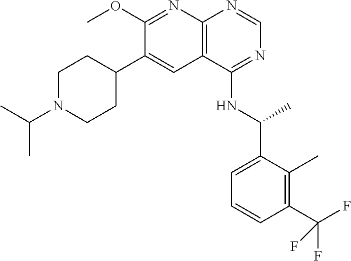 [SMILES: COc1nc2ncnc(N[C@H](C)c3cccc(C(F)(F)F)c3C)c2cc1C1CCN(C(C)C)CC1] | (R)-6-(1-isopropylpiperidin-4-yl)-7- methoxy-N-(1-(2-methyl-3- (trifluoromethyl)phenyl)ethyl)pyrido[2,3- d]pyrimidin-4-amine | 488.3 |
| B349 | 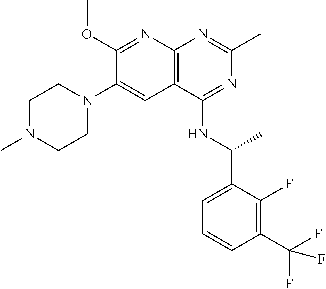 [SMILES: COc1nc2nc(C)nc(N[C@H](C)c3cccc(C(F)(F)F)c3F)c2cc1N1CCN(C)CC1] | (R)-N-(1-(2-fluoro-3- (trifluoromethyl)phenyl)ethyl)-7-methoxy-2- methyl-6-(4-methylpiperazin-1-yl)pyrido[2,3- d]pyrimidin-4-amine | 479.3 |
| B350 | 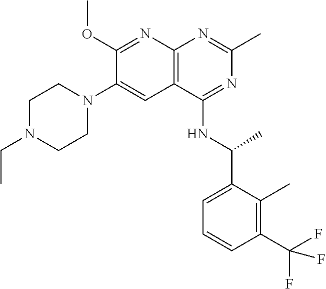 [SMILES: CCN1CCN(c2cc3c(N[C@H](C)c4cccc(C(F)(F)F)c4C)nc(C)nc3nc2OC)CC1] | (R)-6-(4-ethylpiperazin-1-yl)-7-methoxy-2- methyl-N-(1-(2-methyl-3- (trifluoromethyl)phenyl)ethyl)pyrido[2,3- d]pyrimidin-4-amine | 489.4 |
| B351 | 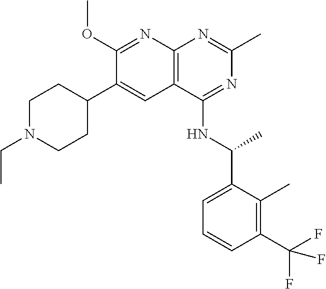 [SMILES: CCN1CCC(c2cc3c(N[C@H](C)c4cccc(C(F)(F)F)c4C)nc(C)nc3nc2OC)CC1] | (R)-6-(1-ethylpiperidin-4-yl)-7-methoxy-2- methyl-N-(1-(2-methyl-3- (trifluoromethyl)phenyl)ethyl)pyrido[2,3- d]pyrimidin-4-amine | 488.4 |
| B352 | 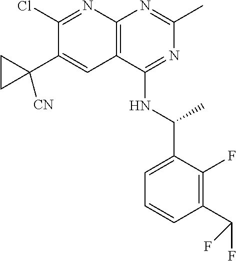 [SMILES: Cc1nc(N[C@H](C)c2cccc(C(F)F)c2F)c2cc(C3(C#N)CC3)c(Cl)nc2n1] | (R)-1-(7-chloro-4-((1-(3-(difluoromethyl)-2- fluorophenyl)ethyl)amino)-2-methylpyrido[2,3- d]pyrimidin-6-yl)cyclopropane-1-carbonitrile | 432.3 |
| B353 | 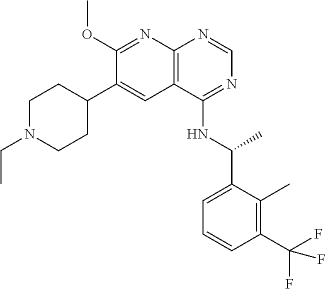 [SMILES: CCN1CCC(c2cc3c(N[C@H](C)c4cccc(C(F)(F)F)c4C)ncnc3nc2OC)CC1] | (R)-6-(1-ethylpiperidin-4-yl)-7-methoxy-N-(1- (2-methyl-3- (trifluoromethyl)phenyl)ethyl)pyrido[2,3- d]pyrimidin-4-amine | 474.3 |
| B354 | 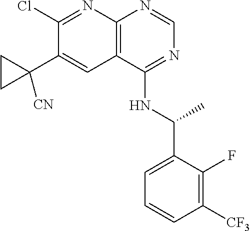 [SMILES: C[C@@H](Nc1ncnc2nc(Cl)c(C3(C#N)CC3)cc12)c1cccc(C(F)(F)F)c1F] | (R)-1-(7-chloro-4-((1-(2-fluoro-3-(tri- fluoromethyl)phenyl)ethyl)amino)pyrido[2,3- d]pyrimidin-6-yl)cyclopropane-1-carbonitrile | 436.5 |
| B355 | 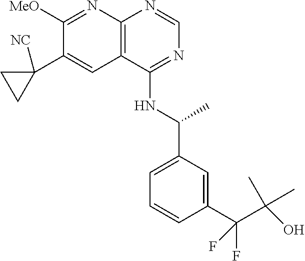 [SMILES: COc1nc2ncnc(N[C@H](C)c3cccc(C(F)(F)C(C)(C)O)c3)c2cc1C1(C#N)CC1] | (R)-1-(4-((1-(3-(1,1-difluoro-2-hydroxy-2- methylpropyl)phenyl)ethyl)amino)-7- methoxypyrido[2,3-d]pyrimidin-6- yl)cyclopropane-1-carbonitrile | 454.7 |
| B356 | 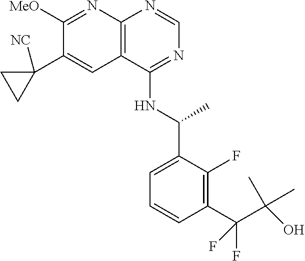 [SMILES: COc1nc2ncnc(N[C@H](C)c3cccc(C(F)(F)C(C)(C)O)c3F)c2cc1C1(C#N)CC1] | (R)-1-(4-((1-(3-(1,1-difluoro-2-hydroxy-2- methylpropyl)-2-fluorophenyl)ethyl)amino)-7- methoxypyrido[2,3-d]pyrimidin-6- yl)cyclopropane-1-carbonitrile | 472.7 |
| B357 | 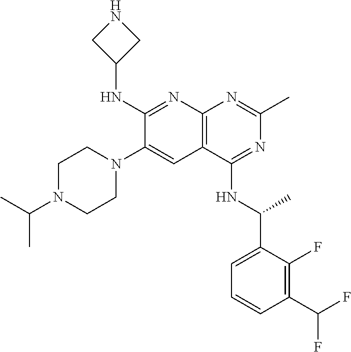 [SMILES: Cc1nc(N[C@H](C)c2cccc(C(F)F)c2F)c2cc(N3CCN(C(C)C)CC3)c(NC3CNC3)nc2n1] | (R)-N7-(azetidin-3-yl)-N4-(1- (3-(difluoromethyl)-2- fluorophenyl)ethyl)-6-(4- isopropylpiperazin-1-yl)-2-methyl- pyrido[2,3-d]pyrimidine-4,7-diamine | 529.3 |
| B358 | 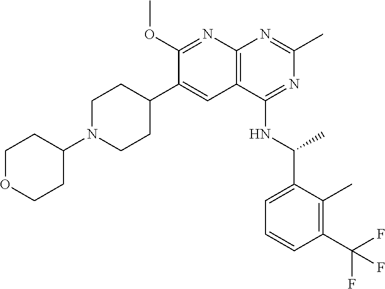 [SMILES: COc1nc2nc(C)nc(N[C@H](C)c3cccc(C(F)(F)F)c3C)c2cc1C1CCN(C2CCOCC2)CC1] | (R)-7-methoxy-2-methyl-N-(1-(2-methyl-3- (trifluoromethyl)phenyl)ethyl)-6- (1-(tetrahydro-2H-pyran-4-yl)piperidin- 4-yl)pyrido[2,3-d]pyrimidin-4-amine | 544.5 |
| B359 | 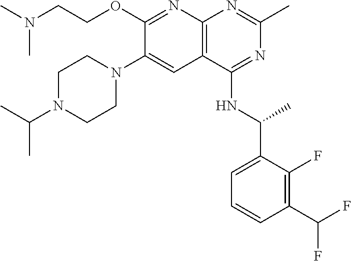 [SMILES: Cc1nc(N[C@H](C)c2cccc(C(F)F)c2F)c2cc(N3CCN(C(C)C)CC3)c(OCCN(C)C)nc2n1] | (R)-N-(1-(3-(difluoromethyl)-2- fluorophenyl)ethyl)-7-(2-(dimethyl- amino)ethoxy)-6-(4-isopropylpiperazin-1-yl)- 2-methylpyrido[2,3-d]pyrimidin-4-amine | 546.3 |
| B360 | 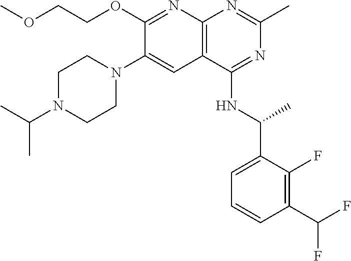 [SMILES: COCCOc1nc2nc(C)nc(N[C@H](C)c3cccc(C(F)F)c3F)c2cc1N1CCN(C(C)C)CC1] | (R)-N-(1-(3-(difluoromethyl)-2-fluoro- phenyl)ethyl)-6-(4-isopropylpiperazin-1-yl)- 7-(2-methoxyethoxy)-2-methylpyrido[2,3- d]pyrimidin-4-amine | 533.5 |
| B361 | 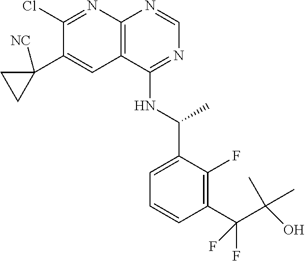 [SMILES: C[C@@H](Nc1ncnc2nc(Cl)c(C3(C#N)CC3)cc12)c1cccc(C(F)(F)C(C)(C)O)c1F] | (R)-1-(7-chloro-4-((1-(3-(1,1-difluoro-2- hydroxy-2-methylpropyl)-2-fluoro- phenyl)ethyl)amino)pyrido[2,3-d]pyrimidin- 6-yl)cyclopropane-1-carbonitrile | 476.2 |
| B362 | 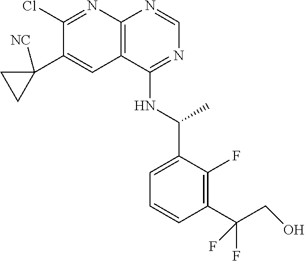 [SMILES: C[C@@H](Nc1ncnc2nc(Cl)c(C3(C#N)CC3)cc12)c1cccc(C(F)(F)CO)c1F] | (R)-1-(7-chloro-4-((1-(3-(1,1-difluoro-2- hydroxyethyl)-2-fluoro- phenyl)ethyl)amino)pyrido[2,3-d]pyrimidin- 6-yl)cyclopropane-1-carbonitrile | 448.2 |
| B363 | 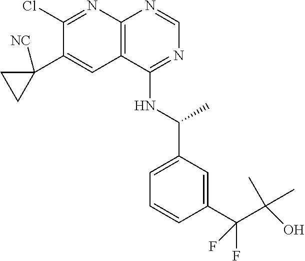 [SMILES: C[C@@H](Nc1ncnc2nc(Cl)c(C3(C#N)CC3)cc12)c1cccc(C(F)(F)C(C)(C)O)c1] | (R)-1-(7-chloro-4-((1-(3-(1,1-difluoro- 2-hydroxy-2-methylpropyl)phenyl)eth- yl)amino)pyrido[2,3-d]pyrimidin- 6-yl)cyclopropane-1-carbonitrile | 458.3 |
| B364 | 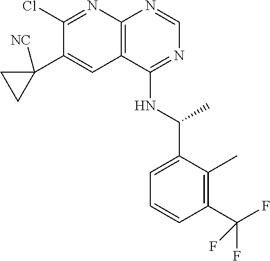 [SMILES: Cc1c([C@@H](C)Nc2ncnc3nc(Cl)c(C4(C#N)CC4)cc23)cccc1C(F)(F)F] | (R)-1-(7-chloro-4-((1-(2-methyl-3-(tri- fluoromethyl)phenyl)ethyl)amino)pyrido[2,3- d]pyrimidin-6-yl)cyclopropane-1-carbonitrile | 432.2 |
| B365 | 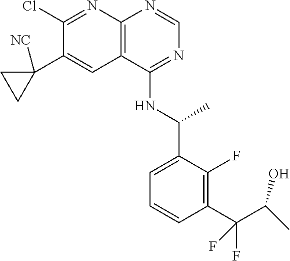 [SMILES: C[C@@H](Nc1ncnc2nc(Cl)c(C3(C#N)CC3)cc12)c1cccc(C(F)(F)[C@@H](C)O)c1F] | 1-(7-chloro-4-(((R)-1-(3-((R)-1,1-difluoro-2- hydroxypropyl)-2-fluoro- phenyl)ethyl)amino)pyrido[2,3-d]pyrimidin- 6-yl)cyclopropane-1-carbonitrile | 462.2 |
| B366 | 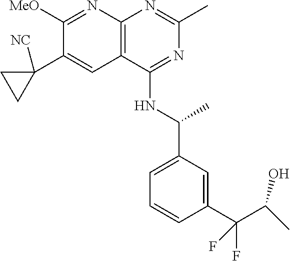 [SMILES: COc1nc2nc(C)nc(N[C@H](C)c3cccc(C(F)(F)[C@@H](C)O)c3)c2cc1C1(C#N)CC1] | 1-(4-(((R)-1-(3-((R)-1,1-difluoro-2- hydroxypropyl)phenyl)ethyl)amino)-7- methoxy-2-methylpyrido[2,3- d]pyrimidin-6-yl)cyclopropane- 1-carbonitrile | 454.3 |
| B367 | 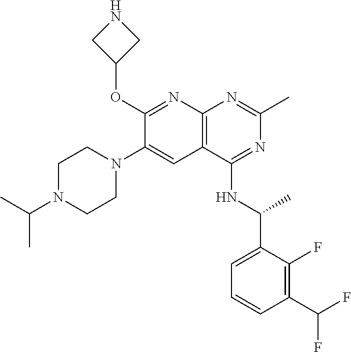 [SMILES: Cc1nc(N[C@H](C)c2cccc(C(F)F)c2F)c2cc(N3CCN(C(C)C)CC3)c(OC3CNC3)nc2n1] | (R)-7-(azetidin-3-yloxy)-N-(1- (3-(difluoromethyl)-2- fluorophenyl)ethyl)-6-(4-isopropylpiperazin- 1-yl)-2-methylpyrido[2,3-d]pyrimidin-4- amine | 530.5 |
| B368 | 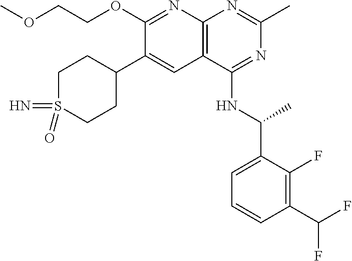 [SMILES: COCCOc1nc2nc(C)nc(N[C@H](C)c3cccc(C(F)F)c3F)c2cc1C1CCS(=N)(=O)CC1] | (R)-4-(4-((1-(3-(difluoromethyl)-2-fluoro- phenyl)ethyl)amino)-7-(2-methoxyethoxy)- 2-methylpyrido[2,3-d]pyrimidin-6-yl)-1- iminohexahydro-1l6-thiopyran 1-oxide | 538.5 |
| B369 | 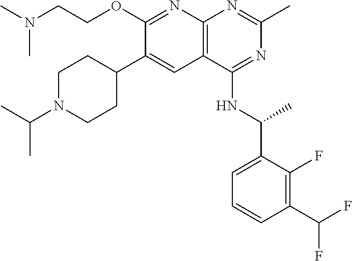 [SMILES: Cc1nc(N[C@H](C)c2cccc(C(F)F)c2F)c2cc(C3CCN(C(C)C)CC3)c(OCCN(C)C)nc2n1] | (R)-N-(1-(3-(difluoromethyl)-2-fluoro- phenyl)ethyl)-7-(2-(dimethylamino)ethoxy)- 6-(1-isopropylpiperidin-4-yl)-2-methylpyri- do[2,3-d]pyrimidin-4-amine | 545.6 |
| B370 | 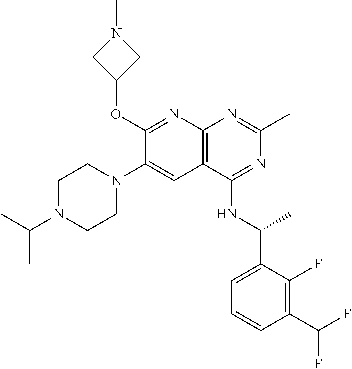 [SMILES: Cc1nc(N[C@H](C)c2cccc(C(F)F)c2F)c2cc(N3CCN(C(C)C)CC3)c(OC3CN(C)C3)nc2n1] | (R)-N-(1-(3-(difluoromethyl)-2-fluoro- phenyl)ethyl)-6-(4-isopropylpiperazin-1-yl)- 2-methyl-7-((1-methylazetidin-3- yl)oxy)pyrido[2,3-d]pyrimidin-4-amine | 544.5 |
| B371 | 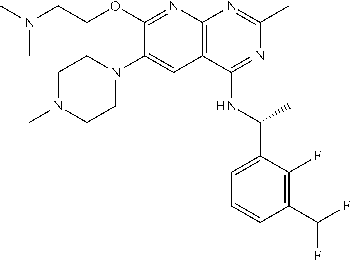 [SMILES: Cc1nc(N[C@H](C)c2cccc(C(F)F)c2F)c2cc(N3CCN(C)CC3)c(OCCN(C)C)nc2n1] | (R)-N-(1-(3-(difluoromethyl)-2-fluoro- phenyl)ethyl)-7-(2-(dimethylamino)ethoxy)- 2-methyl-6-(4-methylpiperazin-1-yl)pyrido[2,3- d]pyrimidin-4-amine | 518.5 |
| B372 | 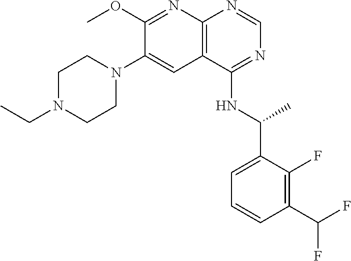 [SMILES: CCN1CCN(c2cc3c(N[C@H](C)c4cccc(C(F)F)c4F)ncnc3nc2OC)CC1] | (R)-N-(1-(3-(difluoromethyl)-2-fluoro- phenyl)ethyl)-6-(4-ethylpiperazin-1-yl)-7- methoxypyrido[2,3-d]pyrimidin-4-amine | 461.3 |
| B373 | 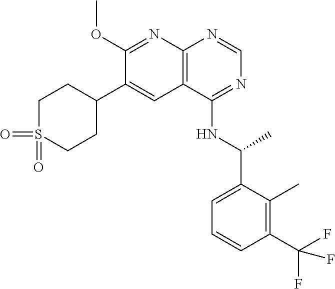 [SMILES: COc1nc2ncnc(N[C@H](C)c3cccc(C(F)(F)F)c3C)c2cc1C1CCS(=O)(=O)CC1] | (R)-4-(7-methoxy-4-((1-(2-methyl-3- (trifluoromethyl)phenyl)ethyl)amino)pyri do[2,3-d]pyrimidin-6-yl)tetrahydro-2H- thiopyran 1,1-dioxide | 530.4 |
| B374 | 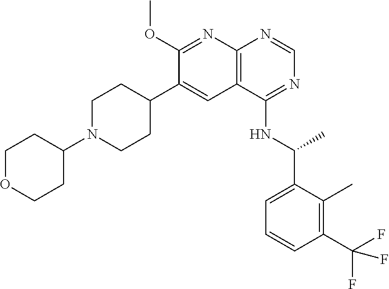 [SMILES: COc1nc2ncnc(N[C@H](C)c3cccc(C(F)(F)F)c3C)c2cc1C1CCN(C2CCOCC2)CC1] | (R)-7-methoxy-N-(1-(2-methyl-3- (trifluoromethyl)phenyl)ethyl)-6-(1-(tetrahydro- 2H-pyran-4-yl)piperidin-4-yl)pyrido[2,3- d]pyrimidin-4-amine | 495.3 |
| B375 | 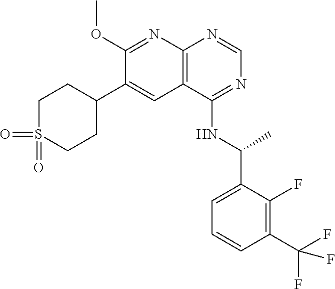 [SMILES: COc1nc2ncnc(N[C@H](C)c3cccc(C(F)(F)F)c3F)c2cc1C1CCS(=O)(=O)CC1] | (R)-4-(4-((1-(2-fluoro-3- (trifluoromethyl)phenyl)ethyl)amino)-7- methoxypyrido[2,3-d]pyrimidin-6-yl)tetrahydro- 2H-thiopyran 1,1-dioxide | 499.3 |
| B376 | 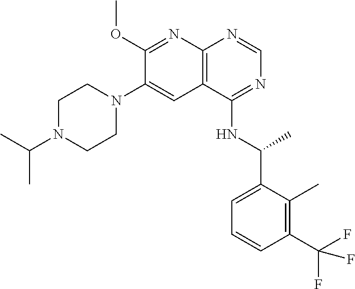 [SMILES: COc1nc2ncnc(N[C@H](C)c3cccc(C(F)(F)F)c3C)c2cc1N1CCN(C(C)C)CC1] | (R)-6-(4-isopropylpiperazin-1-yl)-7-methoxy-N- (1-(2-methyl-3- (trifluoromethyl)phenyl)ethyl)pyrido[2,3- d]pyrimidin-4-amine | 489.4 |
| B377 | 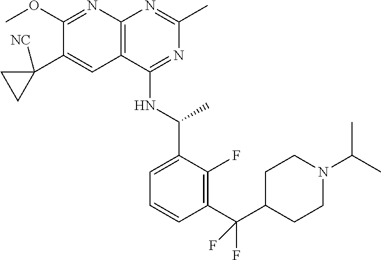 [SMILES: COc1nc2nc(C)nc(N[C@H](C)c3cccc(C(F)(F)C4CCN(C(C)C)CC4)c3F)c2cc1C1(C#N)CC1] | (R)-1-(4-((1-(3-(difluoro(1-isopropylpiperidin-4- yl)methyl)-2-fluorophenyl)ethyl)amino)-7- methoxy-2-methylpyrido[2,3-d]pyrimidin-6- yl)cyclopropane-1-carbonitrile | 533.4 |
| B378 | 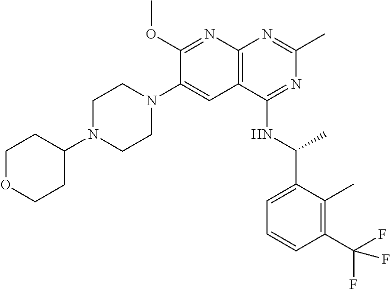 [SMILES: COc1nc2nc(C)nc(N[C@H](C)c3cccc(C(F)(F)F)c3C)c2cc1N1CCN(C2CCOCC2)CC1] | (R)-7-methoxy-2-methyl-N-(1-(2-methyl-3- (trifluoromethyl)phenyl)ethyl)-6-(4-(tetrahydro- 2H-pyran-4-yl)piperazin-1-yl)pyrido[2,3- d]pyrimidin-4-amine | 545.4 |
| B379 | 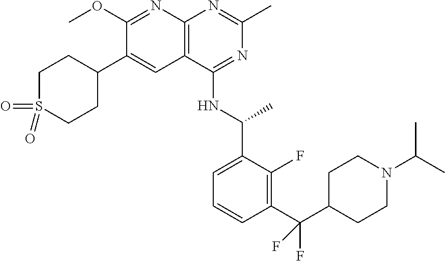 [SMILES: COc1nc2nc(C)nc(N[C@H](C)c3cccc(C(F)(F)C4CCN(C(C)C)CC4)c3F)c2cc1C1CCS(=O)(=O)CC1] | (R)-4-(4-((1-(3-(difluoro(1-isopropylpiperidin-4- yl)methyl)-2-fluorophenyl)ethyl)amino)-7- methoxy-2-methylpyrido[2,3-d]pyrimidin-6- yl)tetrahydro-2H-thiopyran 1,1-dioxide | 620.5 |
| B380 | 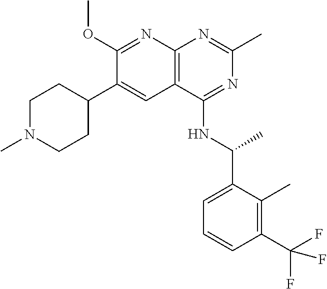 [SMILES: COc1nc2nc(C)nc(N[C@H](C)c3cccc(C(F)(F)F)c3C)c2cc1C1CCN(C)CC1] | (R)-7-methoxy-2-methyl-N-(1-(2-methyl-3- (trifluoromethyl)phenyl)ethyl)-6-(1- methylpiperidin-4-yl)pyrido[2,3-d]pyrimidin-4- amine | 474.4 |
| B381 | 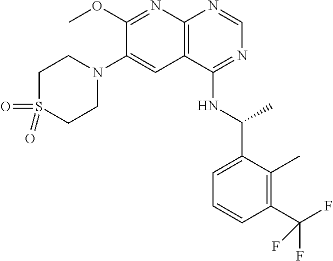 [SMILES: COc1nc2ncnc(N[C@H](C)c3cccc(C(F)(F)F)c3C)c2cc1N1CCS(=O)(=O)CC1] | (R)-4-(7-methoxy-4-((1-(2-methyl-3- (trifluoromethyl)phenyl)ethyl)amino)pyrido[2,3- d]pyrimidin-6-yl)thiomorpholine 1,1-dioxide | 496.7 |
| B382 | 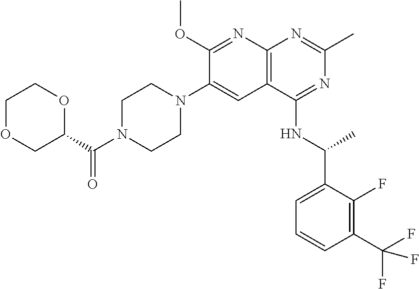 [SMILES: COc1nc2nc(C)nc(N[C@H](C)c3cccc(C(F)(F)F)c3F)c2cc1N1CCN(C(=O)[C@@H]2COCCO2)CC1] | ((S)-1,4-dioxan-2-yl)(4-(4-(((R)-1-(2-fluoro-3- (trifluoromethyl)phenyl)ethyl)amino)-7-methoxy- 2-methylpyrido[2,3-d]pyrimidin-6-yl)piperazin-1- yl)methanone | 579.4 |
| B383 | 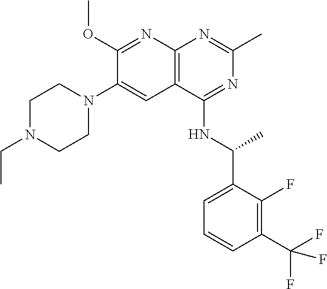 [SMILES: CCN1CCN(c2cc3c(N[C@H](C)c4cccc(C(F)(F)F)c4F)nc(C)nc3nc2OC)CC1] | (R)-6-(4-ethylpiperazin-1-yl)-N-(1-(2-fluoro-3- (trifluoromethyl)phenyl)ethyl)-7-methoxy-2- methylpyrido[2,3-d]pyrimidin-4-amine | 493.3 |
| B384 | 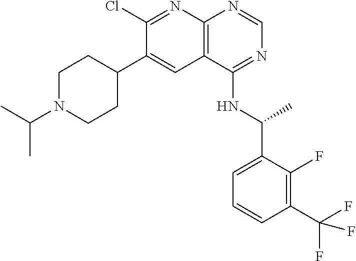 [SMILES: CC(C)N1CCC(c2cc3c(N[C@H](C)c4cccc(C(F)(F)F)c4F)ncnc3nc2Cl)CC1] | (R)-7-chloro-N-(1-(2-fluoro-3- (trifluoromethyl)phenyl)ethyl)-6-(1- isopropylpiperidin-4-yl)pyrido[2,3-d]pyrimidin-4- amine | 496.4 |
| B385 | 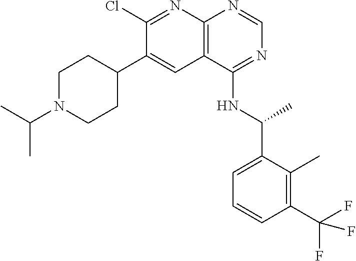 [SMILES: Cc1c([C@@H](C)Nc2ncnc3nc(Cl)c(C4CCN(C(C)C)CC4)cc23)cccc1C(F)(F)F] | (R)-7-chloro-6-(1-isopropylpiperidin-4-yl)-N-(1- (2-methyl-3- (trifluoromethyl)phenyl)ethyl)pyrido[2,3- d]pyrimidin-4-amine | 492.4 |
| B386 | 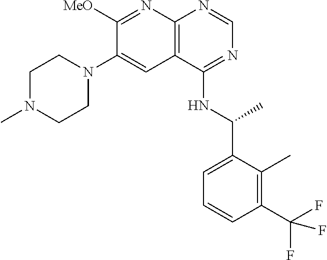 [SMILES: COc1nc2ncnc(N[C@H](C)c3cccc(C(F)(F)F)c3C)c2cc1N1CCN(C)CC1] | (R)-7-methoxy-N-(1-(2-methyl-3- (trifluoromethyl)phenyl)ethyl)-6-(4- methylpiperazin-1-yl)pyrido[2,3-d]pyrimidin-4- amine | 461.4 |
| B387 | 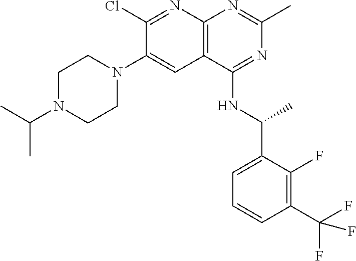 [SMILES: Cc1nc(N[C@H](C)c2cccc(C(F)(F)F)c2F)c2cc(N3CCN(C(C)C)CC3)c(Cl)nc2n1] | (R)-7-chloro-N-(1-(2-fluoro-3- (trifluoromethyl)phenyl)ethyl)-6-(4- isopropylpiperazin-1-yl)-2-methylpyrido[2,3- d]pyrimidin-4-amine | 511.6 |
| B388 | 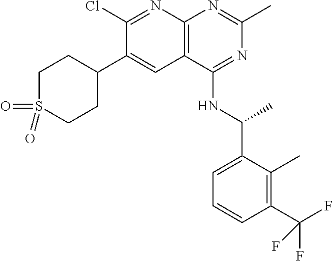 [SMILES: Cc1nc(N[C@H](C)c2cccc(C(F)(F)F)c2C)c2cc(C3CCS(=O)(=O)CC3)c(Cl)nc2n1] | (R)-4-(7-chloro-2-methyl-4-((1-(2-methyl-3- (trifluoromethyl)phenyl)ethyl)amino)pyrido[2,3- d]pyrimidin-6-yl)tetrahydro-2H-thiopyran 1,1- dioxide | 513.2 |
| B389 | 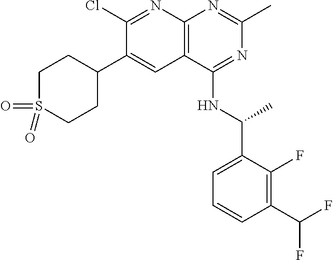 [SMILES: Cc1nc(N[C@H](C)c2cccc(C(F)F)c2F)c2cc(C3CCS(=O)(=O)CC3)c(Cl)nc2n1] | (R)-4-(7-chloro-4-((1-(3-(difluoromethyl)-2- fluorophenyl)ethyl)amino)-2-methylpyrido[2,3- d]pyrimidin-6-yl)tetrahydro-2H-thiopyran 1,1- dioxide | 499.3 |
| B390 | 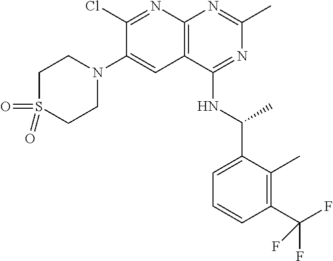 [SMILES: Cc1nc(N[C@H](C)c2cccc(C(F)(F)F)c2C)c2cc(N3CCS(=O)(=O)CC3)c(Cl)nc2n1] | (R)-4-(7-chloro-2-methyl-4-((1-(2-methyl-3- (trifluoromethyl)phenyl)ethyl)amino)pyrido[2,3- d]pyrimidin-6-yl)thiomorpholine 1,1-dioxide | 514.3 |
| B391 | 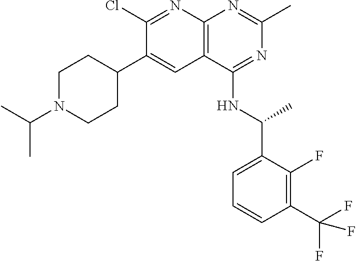 [SMILES: Cc1nc(N[C@H](C)c2cccc(C(F)(F)F)c2F)c2cc(C3CCN(C(C)C)CC3)c(Cl)nc2n1] | (R)-7-chloro-N-(1-(2-fluoro-3- (trifluoromethyl)phenyl)ethyl)-6-(1- isopropylpiperidin-4-yl)-2-methylpyrido[2,3- d]pyrimidin-4-amine | 510.3 |
| B392 | 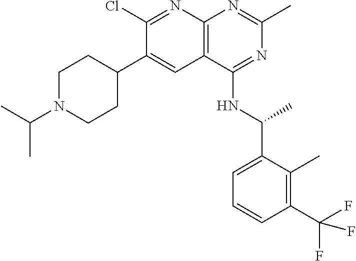 [SMILES: Cc1nc(N[C@H](C)c2cccc(C(F)(F)F)c2C)c2cc(C3CCN(C(C)C)CC3)c(Cl)nc2n1] | (R)-7-chloro-6-(1-isopropylpiperidin-4-yl)-2- methyl-N-(1-(2-methyl-3- (trifluoromethyl)phenyl)ethyl)pyrido[2,3- d]pyrimidin-4-amine | 506.4 |
| B393 | 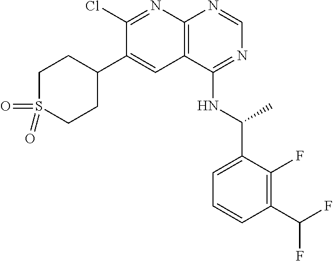 [SMILES: C[C@@H](Nc1ncnc2nc(Cl)c(C3CCS(=O)(=O)CC3)cc12)c1cccc(C(F)F)c1F] | (R)-4-(7-chloro-4-((1-(3-(difluoromethyl)-2- fluorophenyl)ethyl)amino)pyrido[2,3- d]pyrimidin-6-yl)tetrahydro-2H-thiopyran 1,1-dioxide | 485.2 |
| B394 | 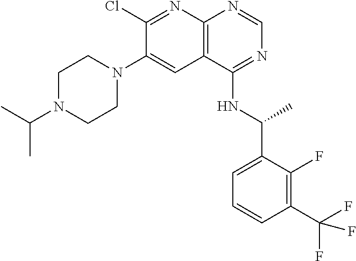 [SMILES: CC(C)N1CCN(c2cc3c(N[C@H](C)c4cccc(C(F)(F)F)c4F)ncnc3nc2Cl)CC1] | (R)-7-chloro-N-(1-(2-fluoro-3- (trifluoromethyl)phenyl)ethyl)-6-(4- isopropylpiperazin-1-yl)pyrido[2,3-d]pyrimidin- 4-amine | 497.4 |
| B395 | 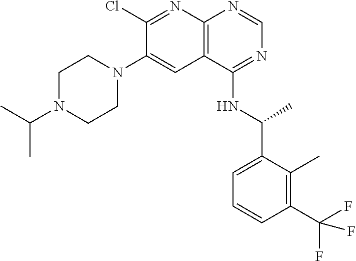 [SMILES: Cc1c([C@@H](C)Nc2ncnc3nc(Cl)c(N4CCN(C(C)C)CC4)cc23)cccc1C(F)(F)F] | (R)-7-chloro-6-(4-isopropylpiperazin-1-yl)-N- (1-(2-methyl-3- (trifluoromethyl)phenyl)ethyl)pyrido[2,3- d]pyrimidin-4-amine | 493.3 |
| B396 | 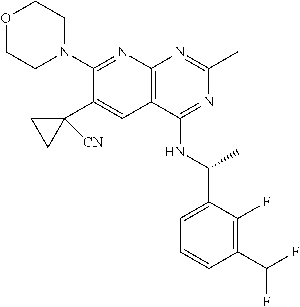 [SMILES: Cc1nc(N[C@H](C)c2cccc(C(F)F)c2F)c2cc(C3(C#N)CC3)c(N3CCOCC3)nc2n1] | (R)-1-(4-((1-(3-(difluoromethyl)-2- fluorophenyl)ethyl)amino)-2-methyl-7- morpholinopyrido[2,3-d]pyrimidin-6- yl)cyclopropane-1-carbonitrile | 483.7 |
| B397 | 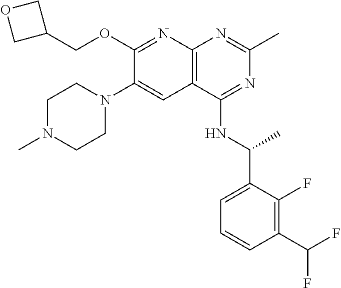 [SMILES: Cc1nc(N[C@H](C)c2cccc(C(F)F)c2F)c2cc(N3CCN(C)CC3)c(OCC3COC3)nc2n1] | (R)-N-(1-(3-(difluoromethyl)-2- fluorophenyl)ethyl)-2-methyl-6-(4- methylpiperazin-1-yl)-7-(oxetan-3- ylmethoxy)pyrido[2,3-d]pyrimidin-4-amine | 517.4 |
| B398 | 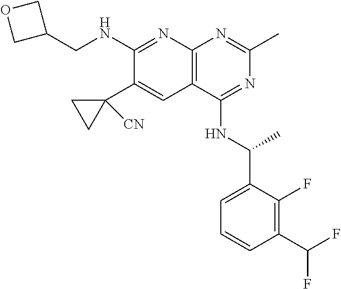 [SMILES: Cc1nc(N[C@H](C)c2cccc(C(F)F)c2F)c2cc(C3(C#N)CC3)c(NCC3COC3)nc2n1] | (R)-1-(4-((1-(3-(difluoromethyl)-2-fluoro- phenyl)ethyl)amino)-2-methyl-7-((oxetan-3- ylmethyl)amino)pyrido[2,3-d]pyrimidin-6- yl)cyclopropane-1-carbonitrile | 483.3 |
| B399 | 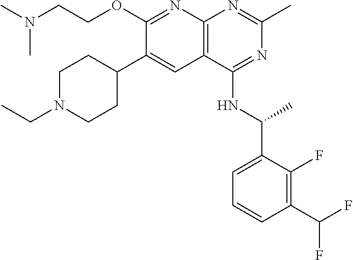 [SMILES: CCN1CCC(c2cc3c(N[C@H](C)c4cccc(C(F)F)c4F)nc(C)nc3nc2OCCN(C)C)CC1] | (R)-N-(1-(3-(difluoromethyl)-2-fluoro- phenyl)ethyl)-7-(2-(dimethylamino)ethoxy)- 6-(1-ethylpiperidin-4-yl)-2-methylpyrido[2,3- d]pyrimidin-4-amine | 531.5 |
| B400 | 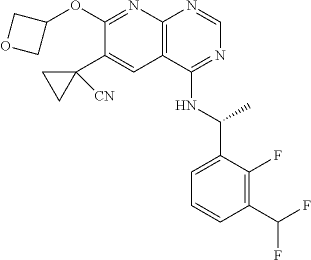 [SMILES: C[C@@H](Nc1ncnc2nc(OC3COC3)c(C3(C#N)CC3)cc12)c1cccc(C(F)F)c1F] | (R)-1-(4-((1-(3-(difluoromethyl)-2- fluorophenyl)ethyl)amino)-7-(oxetan-3- yloxy)pyrido[2,3-d]pyrimidin-6- yl)cyclopropane-1-carbonitrile | 456.7 |
| B401 | 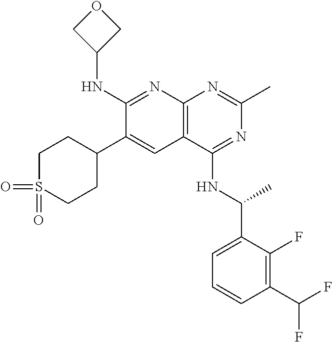 [SMILES: Cc1nc(N[C@H](C)c2cccc(C(F)F)c2F)c2cc(C3CCS(=O)(=O)CC3)c(NC3COC3)nc2n1] | (R)-4-(4-((1-(3-(difluoromethyl)-2- fluorophenyl)ethyl)amino)-2-methyl-7-(oxetan- 3-ylamino)pyrido[2,3-d]pyrimidin- 6-yl)tetrahydro-2H-thiopyran 1,1-dioxide | 536.5 |
| B402 | 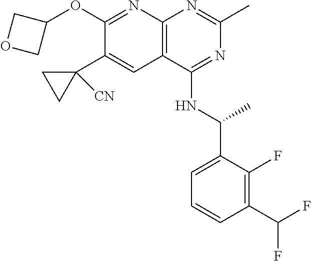 [SMILES: Cc1nc(N[C@H](C)c2cccc(C(F)F)c2F)c2cc(C3(C#N)CC3)c(OC3COC3)nc2n1] | (R)-1-(4-((1-(3-(difluoromethyl)-2- fluorophenyl)ethyl)amino)-2-methyl-7-(oxetan- 3-yloxy)pyrido[2,3-d]pyrimidin-6- yl)cyclopropane-1-carbonitrile | 470.4 |
| B403 | 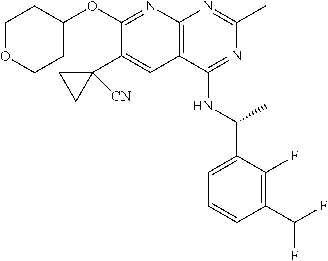 [SMILES: Cc1nc(N[C@H](C)c2cccc(C(F)F)c2F)c2cc(C3(C#N)CC3)c(OC3CCOCC3)nc2n1] | (R)-1-(4-((1-(3-(difluoromethyl)-2- fluorophenyl)ethyl)amino)-2-methyl-7- ((tetrahydro-2H-pyran-4-yl)oxy)pyrido[2,3- d]pyrimidin-6-yl)cyclopropane-1-carbonitrile | 498.3 |
| B404 | 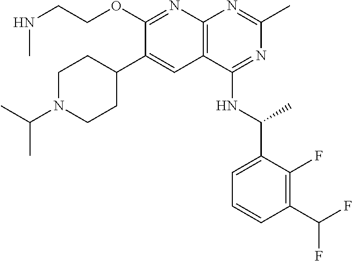 [SMILES: CNCCOc1nc2nc(C)nc(N[C@H](C)c3cccc(C(F)F)c3F)c2cc1C1CCN(C(C)C)CC1] | (R)-N-(1-(3-(difluoromethyl)-2-fluoro- phenyl)ethyl)-6-(1-isopropylpiperidin-4-yl)- 2-methyl-7-(2-(methylamino)ethoxy)pyri- do[2,3-d]pyrimidin-4-amine | 531.4 |
| B405 | 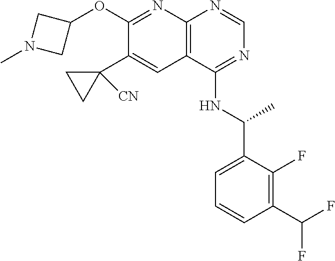 [SMILES: C[C@@H](Nc1ncnc2nc(OC3CN(C)C3)c(C3(C#N)CC3)cc12)c1cccc(C(F)F)c1F] | (R)-1-(4-((1-(3-(difluoromethyl)-2- fluorophenyl)ethyl)amino)-7-((1-methyl- azetidin-3-yl)oxy)pyrido[2,3-d]pyrimidin- 6-yl)cyclopropane-1-carbonitrile | 469.3 |
| B406 | 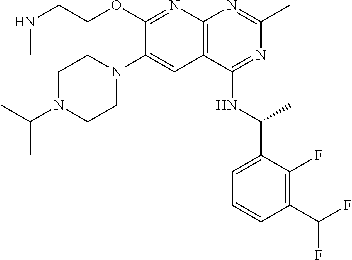 [SMILES: CNCCOc1nc2nc(C)nc(N[C@H](C)c3cccc(C(F)F)c3F)c2cc1N1CCN(C(C)C)CC1] | (R)-N-(1-(3-(difluoromethyl)-2-fluoro- phenyl)ethyl)-6-(4-isopropylpiperazin-1-yl)- 2-methyl-7-(2-(methylamino)ethoxy)pyrido[2,3- d]pyrimidin-4-amine | 532.5 |
| B407 | 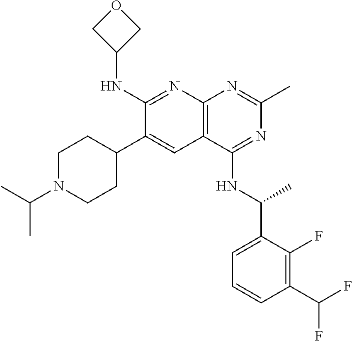 [SMILES: Cc1nc(N[C@H](C)c2cccc(C(F)F)c2F)c2cc(C3CCN(C(C)C)CC3)c(NC3COC3)nc2n1] | (R)-N4-(1-(3-(difluoromethyl)-2-fluoro- phenyl)ethyl)-6-(1-isopropylpiperidin-4-yl)- 2-methyl-N7-(oxetan-3-yl)pyrido[2,3- d]pyrimidine-4,7-diamine | 529.4 |
| B408 | 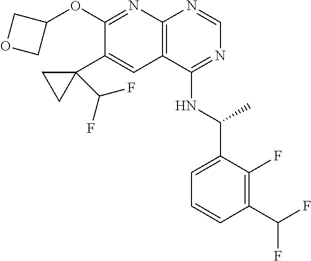 [SMILES: C[C@@H](Nc1ncnc2nc(OC3COC3)c(C3(C(F)F)CC3)cc12)c1cccc(C(F)F)c1F] | (R)-N-(1-(3-(difluoromethyl)-2- fluorophenyl)ethyl)-6-(1- (difluoromethyl)cyclopropyl)-7-(oxetan-3- yloxy)pyrido[2,3-d]pyrimidin-4-amine | 481.2 |
| B409 | 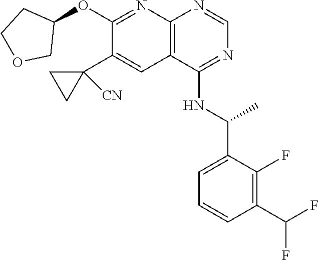 [SMILES: C[C@@H](Nc1ncnc2nc(O[C@@H]3CCOC3)c(C3(C#N)CC3)cc12)c1cccc(C(F)F)c1F] | 1-(4-(((R)-1-(3-(difluoromethyl)-2- fluorophenyl)ethyl)amino)-7-(((R)- tetrahydrofuran-3-yl)oxy)pyrido[2,3- d]pyrimidin-6-yl)cyclopropane-1-carbonitrile | 470.7 |
| B410 | 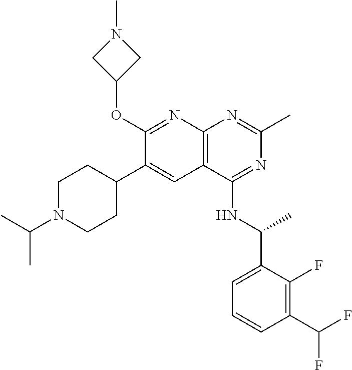 [SMILES: Cc1nc(N[C@H](C)c2cccc(C(F)F)c2F)c2cc(C3CCN(C(C)C)CC3)c(OC3CN(C)C3)nc2n1] | (R)-N-(1-(3-(difluoromethyl)-2- fluorophenyl)ethyl)-6-(1-isopropylpiperidin- 4-yl)-2-methyl-7-((1-methylazetidin-3- yl)oxy)pyrido[2,3-d]pyrimidin-4-amine | 543.5 |
| B411 | 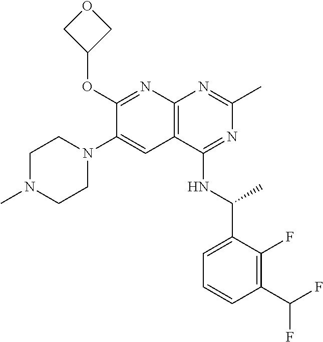 [SMILES: Cc1nc(N[C@H](C)c2cccc(C(F)F)c2F)c2cc(N3CCN(C)CC3)c(OC3COC3)nc2n1] | (R)-N-(1-(3-(difluoromethyl)-2- fluorophenyl)ethyl)-2-methyl-6-(4- methylpiperazin-1-yl)-7-(oxetan-3- yloxy)pyrido[2,3-d]pyrimidin-4-amine | 503.5 |
| B412 | 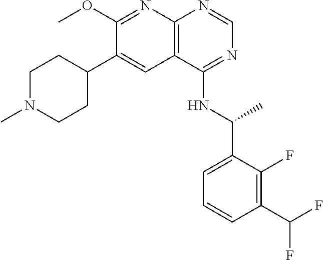 [SMILES: COc1nc2ncnc(N[C@H](C)c3cccc(C(F)F)c3F)c2cc1C1CCN(C)CC1] | (R)-N-(1-(3-(difluoromethyl)-2- fluorophenyl)ethyl)-7-methoxy-6-(1- methylpiperidin-4-yl)pyrido[2,3-d]pyrimidin- 4-amine | 446.3 |
| B413 | 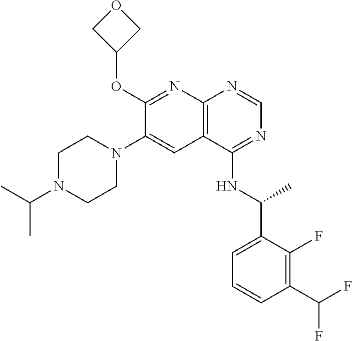 [SMILES: CC(C)N1CCN(c2cc3c(N[C@H](C)c4cccc(C(F)F)c4F)ncnc3nc2OC2COC2)CC1] | (R)-N-(1-(3-(difluoromethyl)-2-fluoro- phenyl)ethyl)-6-(4-isopropylpiperazin-1-yl)- 7-(oxetan-3-yloxy)pyrido[2,3-d]pyrimidin-4- amine | 517.4 |
| B414 | 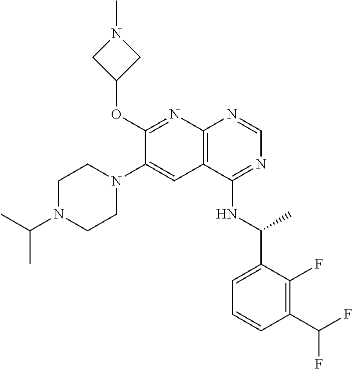 [SMILES: CC(C)N1CCN(c2cc3c(N[C@H](C)c4cccc(C(F)F)c4F)ncnc3nc2OC2CN(C)C2)CC1] | (R)-N-(1-(3-(difluoromethyl)-2- fluorophenyl)ethyl)-6-(4-isopropylpiperazin-1- yl)-7-((1-methylazetidin-3-yl)oxy)pyrido[2,3- d]pyrimidin-4-amine | 530.5 |
| B415 | 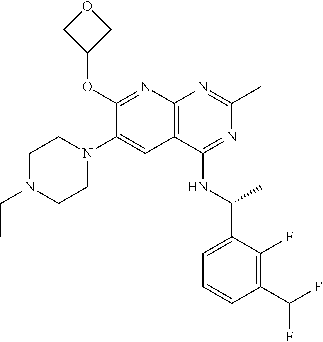 [SMILES: CCN1CCN(c2cc3c(N[C@H](C)c4cccc(C(F)F)c4F)nc(C)nc3nc2OC2COC2)CC1] | (R)-N-(1-(3-(difluoromethyl)-2-fluoro- phenyl)ethyl)-6-(4-ethylpiperazin-1-yl)-2- methyl-7-(oxetan-3-yloxy)pyrido[2,3- d]pyrimidin-4-amine | 517.5 |
| B416 | 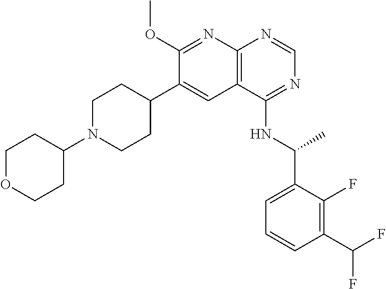 [SMILES: COc1nc2ncnc(N[C@H](C)c3cccc(C(F)F)c3F)c2cc1C1CCN(C2CCOCC2)CC1] | (R)-N-(1-(3-(difluoromethyl)-2-fluoro- phenyl)ethyl)-7-methoxy-6-(1-(tetrahydro- 2H-pyran-4-yl)piperidin-4-yl)pyrido[2,3- d]pyrimidin-4-amine | 516.5 |
| B417 | 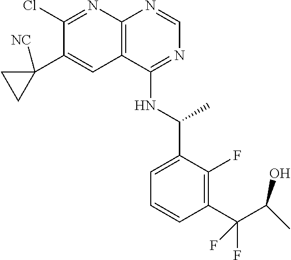 [SMILES: C[C@H](O)C(F)(F)c1cccc([C@@H](C)Nc2ncnc3nc(Cl)c(C4(C#N)CC4)cc23)c1F] | 1-(7-chloro-4-(((R)-1-(3-((S)-1,1-difluoro-2- hydroxypropyl)-2-fluoro- phenyl)ethyl)amino)pyrido[2,3-d]pyrimidin- 6-yl)cyclopropane-1-carbonitrile | 462.3 |
| B418 | 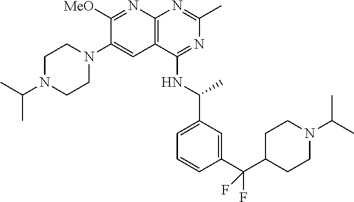 [SMILES: COc1nc2nc(C)nc(N[C@H](C)c3cccc(C(F)(F)C4CCN(C(C)C)CC4)c3)c2cc1N1CCN(C(C)C)CC1] | (R)-N-(1-(3-(difluoro(1-isopropylpiperidin-4- yl)methyl)phenyl)ethyl)-6-(4-isopropylpipera- zin-1-yl)-7-methoxy-2-methylpyrido[2,3- d]pyrimidin-4-amine | 596 |
| B419 | 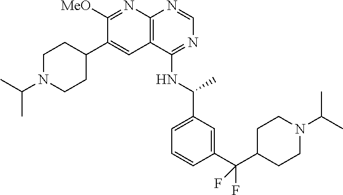 [SMILES: COc1nc2ncnc(N[C@H](C)c3cccc(C(F)(F)C4CCN(C(C)C)CC4)c3)c2cc1C1CCN(C(C)C)CC1] | (R)-N-(1-(3-(difluoro(1-isopropylpiperidin-4- yl)methyl)phenyl)ethyl)-6-(1-isopropylpiperidin- 4-yl)-7-methoxypyrido[2,3-d]pyrimidin- 4-amine | 580 |
| B420 | 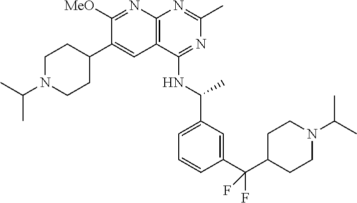 [SMILES: COc1nc2nc(C)nc(N[C@H](C)c3cccc(C(F)(F)C4CCN(C(C)C)CC4)c3)c2cc1C1CCN(C(C)C)CC1] | (R)-N-(1-(3-(difluoro(1-isopropylpiperidin-4- yl)methyl)phenyl)ethyl)-6-(1-isopropylpiperidin- 4-yl)-7-methoxy-2-methylpyrido[2,3- d]pyrimidin-4-amine | 595 |
| B421 | 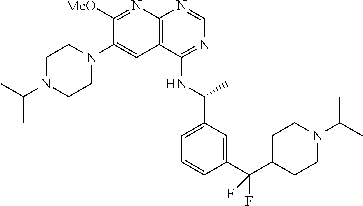 [SMILES: COc1nc2ncnc(N[C@H](C)c3cccc(C(F)(F)C4CCN(C(C)C)CC4)c3)c2cc1N1CCN(C(C)C)CC1] | (R)-N-(1-(3-(difluoro(1-isopropylpiperidin-4- yl)methyl)phenyl)ethyl)-6-(4-isopropylpiperazin- 1-yl)-7-methoxypyrido[2,3-d]pyrimidin-4-amine | 582 |
| B422 | 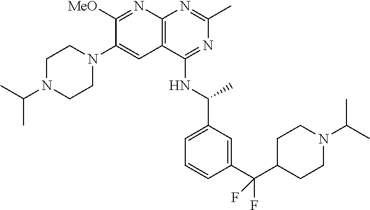 [SMILES: COc1nc2nc(C)nc(N[C@H](C)c3cccc(C(F)(F)C4CCN(C(C)C)CC4)c3)c2cc1N1CCN(C(C)C)CC1] | (R)-N-(1-(3-(difluoro(1-isopropylpiperidin-4- yl)methyl)phenyl)ethyl)-6-(4-isopropylpiperazin- 1-yl)-7-methoxy-2-methylpyrido[2,3- d]pyrimidin-4-amine | 596 |
| B423 | 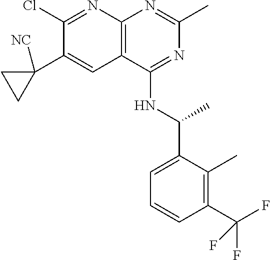 [SMILES: Cc1nc(N[C@H](C)c2cccc(C(F)(F)F)c2C)c2cc(C3(C#N)CC3)c(Cl)nc2n1] | (R)-1-(7-chloro-2-methyl-4-((1-(2-methyl-3- (trifluoromethyl)phenyl)ethyl)amino)pyrido[2,3- d]pyrimidin-6-yl)cyclopropane-1-carbonitrile | 446.2 |
| B424 | 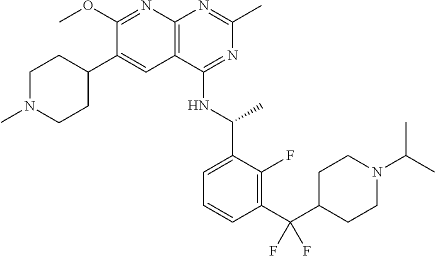 [SMILES: COc1nc2nc(C)nc(N[C@H](C)c3cccc(C(F)(F)C4CCN(C(C)C)CC4)c3F)c2cc1C1CCN(C)CC1] | (R)-N-(1-(3-(difluoro(1-isopropylpiperidin-4- yl)methyl)-2-fluorophenyl)ethyl)-7-methoxy-2- methyl-6-(1-methylpiperidin-4-yl)pyrido[2,3- d]pyrimidin-4-amine | 585.5 |
| B425 | 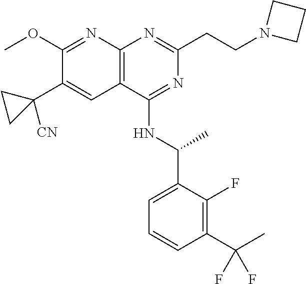 [SMILES: COc1nc2nc(CCN3CCC3)nc(N[C@H](C)c3cccc(C(C)(F)F)c3F)c2cc1C1(C#N)CC1] | (R)-1-(2-(2-(azetidin-1-yl)ethyl)-4-((1-(3-(1,1- difluoroethyl)-2-fluorophenyl)ethyl)amino)-7- methoxypyrido[2,3-d]pyrimidin-6- yl)cyclopropane-1-carbonitrile | 511.4 |
| B426 | 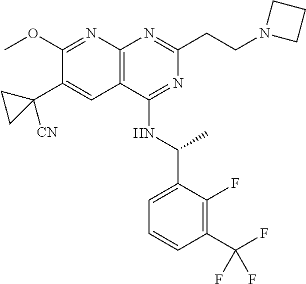 [SMILES: COc1nc2nc(CCN3CCC3)nc(N[C@H](C)c3cccc(C(F)(F)F)c3F)c2cc1C1(C#N)CC1] | (R)-1-(2-(2-(azetidin-1-yl)ethyl)-4-((1-(2-fluoro- (3-trifluoromethyl)phenyl)ethyl)amino)-7- methoxypyrido[2,3-d]pyrimidin-6- yl)cyclopropane-1-carbonitrile | 515.4 |
| B427 | 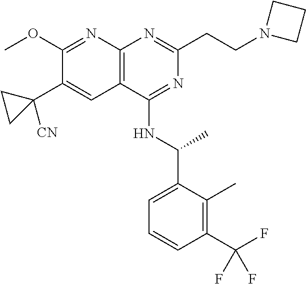 [SMILES: COc1nc2nc(CCN3CCC3)nc(N[C@H](C)c3cccc(C(F)(F)F)c3C)c2cc1C1(C#N)CC1] | (R)-1-(2-(2-(azetidin-1-yl)ethyl)-7-methoxy-4- ((1-(2-methyl-3- (trifluoromethyl)phenyl)ethyl)amino)pyrido[2,3- d]pyrimidin-6-yl)cyclopropane-1-carbonitrile | 511.4 |

In some embodiments, the compounds of the present disclosure exhibit one or more functional characteristics described herein. For example, a subject compound is capable of reducing Ras signaling output. In some instances, a subject compound is capable of disrupting a Ras-SOS interaction, including disrupting interaction or binding between a mutant Kras (e.g., Kras G12C) and SOS1, or between a wildtype Kras and SOS1, thereby reducing Ras signaling output. In some embodiments, a subject compound binds specifically to a SOS protein, including SOS1. In some embodiments, the IC50 of a subject compound (including those shown in Table 1) for a SOS protein is less than about 5 μM, less than about 1 μM, less than about 50 nM, less than about 10 nM, less than about 1 nM, less than about 0.5 nM, less than about 100 μM, or less than about 50 μM, as measured in an in vitro assay known in the art or exemplified herein.

A reduction in Ras signaling output can be evidenced by one or more members of the following: (i) an increase in steady state level of GDP-bound Ras protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, (vi) reduction (e.g., inhibition) of cell growth of Ras-driven tumor cells (e.g., those derived from a tumor cell line disclosed herein), and (vii) an interference or disruption of the interaction or binding between a SOS protein (e.g., SOS1) with a Ras protein such as a wildtype or a mutant Ras. In some cases, the reduction in Ras signaling output can be evidenced by two, three, four, five, six, or all of (i)-(vii) above.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the compounds section of the present disclosure may be utilized in the methods section including methods of use and production disclosed herein, or vice versa.

### Methods

The SOS1 inhibitors and the pharmaceutical compositions thereof, as disclosed herein, are particularly useful in treating Philadelphia chromosome-positive (Ph+) diseases and the symptoms associated therewith. The Philadelphia chromosome abnormality resulting from reciprocal translocation of t(9; 22) (q34; q11) generates a fused BCR-ABL gene that is associated with several types of cancer. Amongst them are blood cancers including leukemia and lymphoma, and secondary cancers in other bodily tissues and organs due to metastasis of the primary Ph+ cancers. Chronic myelogenous leukemia (CML) is the most common Ph+ disease, followed by Ph+ acute lymphoblastic leukemia (Ph+ALL) and Ph+ lymphoblastic lymphoma (Ph+LBL). The compositions and methods disclosed herein open a new avenue for remission or treatment free remission (TFR) for (Ph+) blood cancers including CML.

In an aspect, the present disclosure provides a method of treating one or more of these Philadelphia chromosome-positive (Ph+) blood cancers or ameliorating the symptoms thereof in a subject in need thereof. The method may comprise administering a pharmaceutical composition comprising an effective amount of a small molecule SOS1 inhibitor to the subject, wherein said subject has been treated with a tyrosine kinase inhibitor against BCR-ABL tyrosine kinase (TKI) prior to said administration of the SOS1 inhibitor.

In another aspect, a method of treating Philadelphia chromosome-positive (Ph+) blood cancer in a subject in need thereof comprises administering a pharmaceutical composition comprising an effective amount of a small molecule SOS1 inhibitor, wherein the subject is identified as having a BCR-ABL genetic aberration.

In some embodiments, a subject suitable for a subject treatment method has been previously treated by one or more TKIs. In some embodiments, a subject suitable for a treatment method disclosed herein has been treated with one or more known TKIs, including a first generation TKI (e.g., imatinib), a second generation TKI (e.g., dasatinib), and/or a third generation TKI (e.g., asciminib or ponatinib). Available TKIs may bind to the ATP-binding site in BCR-ABL tyrosine kinase and reduce its enzymatic activity and/or expression level. In some embodiments, exemplary TKIs exhibit an IC50 against BCR-ABL of less than 1 μM, 0.5 μM, 100 nM, 10 nM, 1 nM, 100 μM, or even less. Non-limiting examples of TKIs are imatinib, dasatinib, nilotinib, bosutinib, radotinib, flumatinib, ponatinib, olverembatinib, and asciminib. In some embodiments, the present disclosure provides a method of treating a blood cancer that is resistant and/or intolerant to treatment with dasatinib in a subject in need thereof, the method comprising administering to the subject a SOS1 inhibitor. In some embodiments, the method further comprises administering to the subject dasatinib. In some embodiments, the present disclosure provides a method of treating a blood cancer, such as Ph+ CML in chronic phase (CP), wherein the blood cancer is resistant to, intolerant to, or has progressed on at least two TKIs, the method comprising administering to the subject a SOS1 inhibitor. In some embodiments, the blood cancer is relapsed and/or refractory Ph+ CML-CP. In some embodiments, the at least two TKIs are selected from second generation TKIs (e.g., dasatinib, nilotinib, or bosutinib) and third generation TKIs (e.g., asciminib or ponatinib). In some embodiments, the method further comprises administering to the subject a TKI, such as dasatinib. In some embodiments, the present disclosure provides a method of treating CML-CP, such as CML-CP with warning or failure according to ELN 2020 guidelines, while on dasatinib, the method comprising administering to the subject (a) a SOS1 inhibitor and (b) dasatinib. (For ELN 2020 guidelines, see Hochhaus et al, Leukemia, 2020, 34(4), 966-984 (doi: 10.1038/s41375-020-0776-2), incorporated herein by reference).

Despite the remarkable progress via the advent of several generations of TKIs, drug resistance and/or intolerance to the earlier generations of TKIs severely limit treatment options to subjects with relapses or with resistance to one or more TKI. A subject resistant to a TKI may be characterized by having one or more mutations in the subject's BCR-ABL gene, an overexpressed BCR-ABL gene, a change in drug transporter activity (e.g., increased efflux and decreased influx of TKI), activation of compensatory signaling pathway (e.g., PI3K/AKT/JAK/STAT, and RAS/MAPK), a change in cell metabolism (e.g., change in leukemia stem-cell metabolism as evidenced by metabolic shift, Hypoxia/HIF-1 alpha, Alox5/beta-catenin), epigenic alteration (e.g., mutations on epigenetic regulating genes such as DNMT3A and/or increased methylation of p15 and EBF2 genes), a change of microenvironment and immunological status (e.g., immunosuppressive bone marrow microenvironment (BMM) with increased MDSCs and Treg, plus exhausted T cells), altered expression of microRNAs (e.g., miR-17 and miR-203), and/or alteration of DNA damage repair and genomic instability (e.g., increasing the additional chromosome abnormalities (ACAs) and point mutations). A combination therapy of the present disclosure (e.g., comprising (a) a small molecule SOS1 inhibitor and (b) a tyrosine kinase inhibitor against BCR-ABL tyrosine kinase (TKI)) may result in greater signaling inhibition downstream from BCR-ABL, such as greater inhibition of one or more of STAT5, GAB1/2, ERK, AKT, and S6, than signaling inhibition observed following treatment with a TKI alone, optionally assessed by measuring levels of phosphorylated protein (e.g., p-BCR-ABL, p-STAT5, p-GAB1/2, p-ERK, p-AKT, and p-S6).

In some embodiments, a subject resistant to a TKI comprises one or more mutations in BCR-ABL gene resulting in a decrease in TKI binding affinity to BCR-ABL tyrosine kinase. In some embodiments, a subject resistant to a TKI comprises a mutation in the P-loop (ATP-binding site), a mutation in C-loop (catalytic domain), a mutation in A-loop on the activation domain, a mutation in myristate pocket, and/or a mutation that directly affects the binding of the TKI (drug contact site). In some embodiments, a subject resistant to a TKI comprises a mutation in BCR-ABL gene including but not limited to point mutations selected from the group consisting of T315I, F359V, Y253H, E255K, M351T, G250E, F359I, and H396R. In some embodiments, a subject resistant to a TKI comprises a mutation in BCR-ABL described in Cancer (Basel) 2021, October; 13(19): 4820, which is incorporated herein by reference.

In some embodiments, a subject suitable for a subject treatment method exhibits intolerance to a TKI. TKIs are known to affect multiple organs in the body including the lungs, liver, gastrointestinal tract, kidneys, thyroid, blood, and skin. Cardiac involvement accounts for some of the most serious complications. A subject intolerant to a TKI may suffer from cardiovascular side effects ranging from hypertension, atrial fibrillation, reduced cardiac function, heart failure, and sudden death. A subject may also suffer from other side effects including nausea, vomiting and/or diarrhea, muscle cramps and bone pain, fatigue, rashes, and/or edema, any and all of which may render the subject intolerant to a TKI treatment. In some embodiments, the subject suffers from one or more side effects selected from fluid retention, headache, diarrhea, fatigue, dyspnea, musculoskeletal pain, nausea, skin rash, myalgia, arthralgia, infection, abdominal pain, hemorrhage, pruritis, pain, constipation, myelosuppression, bleeding-related events, cardiovascular toxicity, pulmonary arterial hypertension, QT prolongation, dermatologic reactions, Stevens-Johnson syndrome, erythema multiforme, tumor lysis syndrome, and hepatotoxicity.

A subject resistant to TKI may exhibit progression of the Philadelphia chromosome-positive (Ph+) blood cancer despite the treatment with TKI. Exemplary Philadelphia chromosome-positive (Ph+) blood cancers are chronic myelogenous leukemia (CML), Ph+ acute lymphoblastic leukemia (Ph+ALL), and Ph+ lymphoblastic lymphoma (Ph+LBL). These blood cancers manifest one or more symptoms including fatigue or weakness-such as shortness of breath while doing everyday activities-fever, paleness, bruising, excessive sweating (especially at night), weight loss, nausea and vomiting, headaches, blurred vision, abnormal blood cell counts, abdominal swelling or discomfort due to an enlarged spleen, itching, bone pain, and bleeding. A Ph+ALL subject may also exhibit enlargement of the liver, spleen, and lymph nodes.

Other symptoms associated with a Ph+ blood cancer include abnormal hematological response such as abnormal levels of red blood cells, white blood cells, or platelets detected in a subject. One hematological hallmark of CML is the uncontrolled production of mature and maturing granulocytes or blasts in a subject's bone marrow (for example, the subject's granulocyte count is elevated, e.g., beyond 50,000/mcL (≤50×10⁹/L), or 200,000/mcL (200×10⁹/L) to 1,000,000/mcL (1,000×10⁹/L)). In some embodiments, the subject to be treated by a subject method has a CML in a chronic phase (e.g., typically having less than 10% blasts in the subject's blood or bone marrow). In some embodiments, the subject's CML relapsed into an accelerated phase (e.g., typically having 15% or more but fewer than 30% blasts). In some embodiments, the subject's CML relapsed into a terminal blast phase (e.g., typically having more than 30% blasts, which may have spread to tissues and organs other than bone marrow).

Additional symptoms associated with a Ph+ blood cancer include abnormal molecular responses. Abnormal molecular responses include but are not limited to the presence of BCR-ABL gene in the subject's biological samples including blood, bone marrow, and spinal fluid. In some instances, more than 10%, 20%, 30%, 50%, 60%, 70%, 80%, or 90% of the blood cells comprise the BCR-ABL gene.

Yet additional symptoms associated a Ph+ blood cancer include abnormal cytogenetic responses, one hallmark of which is the presence of Philadelphia chromosome (Ph+ chromosome) in a subject's cells. In some instances, a subject in need of the treatment has Ph+ chromosome present in more than 95%, 80%, 70%, 60%, 50%, 40%, 30%, 20%, 10%, 5%, or 1% of the blood cells. In some instances, a subject in need of the treatment has Ph+ chromosome present in more than 95% of the blood cells, between about 66%-95% of the blood cells, between about 36%-65% of the blood cells, or between about 1%-35% of the blood cells.

Progression may be established for a lack of reduction in any or some of the symptoms disclosed herein or known in the art that are associated with Ph+ blood cancer. In some embodiments, progression is established for a lack of reduction in one or more symptoms after, for example, 3, 4, 5, 6, 7, 8, 9, 10, 11, 12, or more months from the initial treatment of TKI.

In some embodiments, a subject suitable for a subject treatment method suffers from a relapse of a Philadelphia chromosome-positive (Ph+) blood cancer. In some embodiments, the subject suffers from a relapse of CML. In some embodiments, the subject suffers from a relapse of Ph+ALL. In some embodiments, the subject suffers from a relapse of Ph+LBL. A relapse can be established by a renewed onset of any of the symptoms associated with the Philadelphia chromosome-positive (Ph+) blood cancer with which the subject has previously been diagnosed. A relapse may occur, e.g., 3, 5, 7, 8, 9, 10, 11, 12, 15, 18, 24, 25, 26, 27, 28, 29, 30, 36, or more months after the initial treatment of TKI. A relapse may be established by one or more of (a) loss of complete hematologic response (CHR), (b) loss of complete response (CCyR), (c) loss of partial response (PCyR), (d) development of new mutations, (e) loss of major molecular response (MMR), (f) clonal chromosomal abnormalities (CCA), and (g) Ph+.

A variety of techniques are available in the art for assessing a relapse or progression of a Ph+ blood cancer. In particular, techniques are established for detecting abnormal hematological response, molecular response, and/or cytogenetic response. Blood tests on a subject's blood sample may reveal the abnormal level of white blood cells, red blood cells, or platelets, as well as the presence of blasts. Bone marrow tests can be applied to bone marrow aspiration or bone marrow biopsy for detecting the types of leukemia cells (B or T lymphocytes), and their maturation stages. Imaging tests including an X-Ray, a computerized tomography (CT) scan, magnetic resonance imaging (MRI), and an ultrasound scan can detect signs of metastasis of cancer to the brain, spinal cord, or other parts of the body. Spinal fluid tests can be performed to detect any spread of cancer to spinal fluid.

In an aspect, the present disclosure provides a method of treating Philadelphia chromosome-positive (Ph+) blood cancer in a subject. The method comprises: (1) assessing for presence of a genetic aberration associated with resistance to a tyrosine kinase inhibitor against BCR-ABL tyrosine kinase (TKI) in a biological sample that comprises nucleic acid molecules from the subject; and (2) upon detecting said presence of the genetic aberration in BCR-ABL, administering to the subject a pharmaceutical composition comprising an effective amount of a small molecule SOS1 inhibitor.

The presence of Ph+ chromosome or genetic aberration in BCR-ABL can be determined by a wide variety of nucleic acid assays. Exemplary nucleic acid assays include but are not limited to genotyping assays and sequencing methods. Sequencing methods can include next-generation sequencing, targeted sequencing, exome sequencing, whole genome sequencing, massively parallel sequencing, and the like. Several platforms for next generation sequencing are commercially available, including those marketed by Illumina and Pacific Biosciences. Additional nucleic acid assays include but not limited to in situ hybridization (e.g., FISH), polymerase chain reaction (PCR), quantitative PCR (qPCR), quantitative real-time PCR (qRT-PCR), and ligase chain reaction (LCR), all of which are applicable for detecting a genetic aberration in BCR-ABL. Where desired, one or more nucleic acids can be employed to detect a genetic aberration resulting in formation of BCR-ABL gene including but not limited to translation, as well as genetic mutations within the BCR-ABL gene via point mutation, insertion, deletion, or frameshift.

In some embodiments, the step of assessing for presence of a genetic aberration associated with resistance to a TKI detects overexpression of BCR-ABL gene. In some embodiments, the step of assessing detects a mutation in the P-loop (ATP-binding site), in C-loop (catalytic domain), on the A-loop (activation loop), or in myristate pocket. In some embodiments, the step of assessing detects a genetic aberration associated with (i) a change in drug transporter activity (e.g., increased efflux and decreased influx of TKI, thereby confirming drug resistance), (ii) activation of compensatory signaling pathway (e.g., PI3K/AKT/JAK/STAT, and RAS/MAPK), (iii) a change in cell metabolism (e.g., change in leukemia stem-cell metabolism as evidenced by metabolic shift, Hypoxia/HIF-1alpha, Alox5/beta-catenin), (iv) epigenic alteration (e.g., mutations on epigenetic regulating genes such as DNMT3A and/or increased methylation of p15 and EBF2 genes), (v) a change of microenvironment and immunological status (e.g., immunosuppressive bone marrow microenvironment (BMM) with increased MDSCs and Treg, plus exhausted T cells), (vi) altered expression of microRNAs (e.g., miR-17 and miR-203), and/or (vii) alteration of DNA damage repair and genomic instability (e.g., increasing the additional chromosome abnormalities (ACAs) and point mutations).

In some embodiments, the step of assessing detects a mutation in BCR-ABL including but not limited point mutations selected from the group consisting of T315I, F359V, Y253H, E255K, M351T, G250E, F359I, and H396R. In some embodiments, the step of assessing detects one or more mutations in BCR-ABL described in Cancer (Basel) 2021, October; 13(19): 4820, which is incorporated herein by reference. In some embodiments, the step of assessing detects one or more mutations in BCR-ABL that are associated with a decrease in TKI binding affinity to BCR-ABL tyrosine kinase.

A variety of techniques for protein analysis can also be applied for assessing the presence and/or overexpression of BCR-ABL or mutants thereof, as well as expression of other proteins associated with TKI resistance. Suitable protein assays include, without limitation, immunohistochemistry (IHC), ELISA (enzyme linked immunosorbent assays), “sandwich” immunoassays, immunoradiometric assays, in situ immunoassays, western blot analysis, immunoprecipitation assays, immunofluorescent assays, flow cytometry, confocal microscopy, enzymatic assays, surface plasmon resonance, and PAGE-SDS. One or more of these protein assays utilize antibodies or fragments thereof that exhibit specific binding to BCR-ABL polypeptides. A large number of anti-BCR-ABL antibodies are available, including those provided by Thermofisher and Abcam. Commercially available antibodies can also be used in immunoassays to ascertain a decrease in TKI binding affinity to BCR-ABL as a result of a genetic mutation in the BCR-ABL gene.

Of interest for detection by a protein assay are proteinaceous molecules involved in a change in drug transporter activity (either increasing efflux of a TKI or decreasing influx of the TKI), activation of compensatory signaling pathway, change in cell metabolism, epigenic alteration, microenvironment and immunological status, altered expression of microRNAs, or alteration of DNA damage repair and genomic instability.

In practicing a subject method, the presence of Ph+ chromosome, BCR-ABL gene, mutations within the BCR-ABL gene, or other genetic aberrations associated with resistance to a TKI, can be determined using any biological sample comprising the target cells (e.g., cancer cells from a subject under investigation) or constituents thereof (e.g., constituents such as cfDNA from the tumor tissue or cancer cells). The biological sample may be a liquid biological sample or a solid biological sample (in case of metastasis to other tissues) from the subject under investigation or treatment. The biological sample may be a biopsy sample that is fixed, paraffin-embedded, fresh, or frozen. The biological sample may be obtained by any suitable means, including but not limited to blood draw, needle aspiration, fine needle aspiration, core needle biopsy, vacuum assisted biopsy, large core biopsy, incisional biopsy, excisional biopsy, punch biopsy, shave biopsy, skin biopsy, surgical specimen, and venipuncture.

Depending on the tumor tissue or cancer cells to be treated, the biological sample can be obtained from, without limitation, blood or plasma, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues of the subject.

In some embodiments, a biological sample comprises cell-free DNA (cfDNA) derived from a whole blood or plasma of the subject. A sample may be analyzed directly for its contents, or may be processed to purify one or more of its contents for analysis. In some embodiments, one or more components are purified from the sample for detecting the presence of Ph+ chromosome, BCR-ABL gene, mutations within the BCR-ABL gene, or other genetic aberration associated with resistance to a TKI. In some embodiments, the purified component of the biological sample is protein (e.g. total protein, cytoplasmic protein, or membrane protein). In some embodiments, the purified component of the sample is a nucleic acid, such as DNA (e.g. genomic DNA, cDNA, ctDNA, or cfDNA) or RNA (e.g. total RNA, mRNA, or microRNA).

In practicing a subject method disclosed herein, administrating a SOS1 inhibitor or a pharmaceutical composition comprising an effective amount of a SOS1 inhibitor typically involves contacting a cell with a SOS1 inhibitor disclosed herein. The SOS1 inhibitor can be a small molecule, a nucleic acid agent, or a polypeptide (e.g., an endonuclease). The contacting, as disclosed herein, can occur in vitro, ex vivo, or in vivo.

In some embodiments, the cell is contacted with (i) a SOS1 inhibitor disclosed herein and (ii) at least one additional agent. Contacting with (i) and (ii) can take place conjunctively. For example, a SOS1 inhibitor can be administered prior to, subsequent to, or concurrently with the administration of at least one additional agent. For simultaneous contacting, the SOS1 inhibitor and the at least one additional inhibitor can be in the same composition (e.g., as a formulation or as a unit dosage) or in different compositions (e.g., subjecting the cell to two different compositions at the same time). For sequential contacting, the SOS1 inhibitor and the at least one additional agent can be in the same composition (e.g., a single composition exhibiting different release profiles or in different compositions). For sequential contacting, a first contacting of the cell (e.g., with the SOS1 inhibitor or the at least one additional agent) and a second contacting of the cell (e.g., with the at least one additional agent or the SOS1 inhibitor) can be separated by at least about 10 minutes, 20 minutes, 30 minutes, 40 minutes, 50 minutes, 60 minutes, 2 hours, 3 hours, 4 hours, 5 hours, 6 hours, 7 hours, 8 hours, 12 hours, 16 hours, 24 hours, 1 day, 3 days, 5 days, 1 week, 2 weeks, 3 weeks, 1 month, 2 months, or longer. The first contacting and the second contacting can be separated by at most about 1 month, 1 week, 24 hours, 20 hours, 16 hours, 12 hours, 11 hours, 10 hours, 9 hours, 8 hours, 7 hours, 6 hours, 5 hours, 4 hours, 3 hours, 2 hours, 60 minutes, or less.

Suitable agents that can be administered in combination with a subject SOS1 inhibitor include but are not limited to TKIs disclosed herein, other anti-cancer agents, anti-allergic agents, anti-nausea agents (or anti-emetics), pain relievers, cytoprotective agents, immunomodulatory agents, chemotherapeutic agents, and combinations thereof.

Exemplary immunomodulatory agents include but are not limited to immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-1BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1BB antibody. In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-H3 antibodies (e.g., DS-5573a), anti-TIM3 antibodies (e.g., F38-2E2), and combinations thereof. In one embodiment, the checkpoint immune blockade agent is an anti-PD-L1 antibody.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, a method described herein that comprises administering (a) a SOS1 inhibitor and optionally (b) a TKI, such as dasatinib, further comprises administering (c) an additional agent selected from (1) a SHP2 inhibitor (e.g., 6-(4-amino-4-methylpiperidin-1-yl)-3-(2,3-dichlorophenyl)pyrazin-2-amine, RMC-4630, ERAS-601, TNO155, JAB-3068, IACS-13909/BBP-398, SHP099, RMC-4550), (2) an inhibitor of wildtype or mutant RAS, such as wildtype KRAS, wildtype HRAS, wildtype NRAS, mutant KRAS, mutant HRAS, mutant NRAS, KRAS G12C, KRAS G12D, KRAS G12S, KRAS G12V, KRAS G13D, KRAS G13C, KRAS G13V, or KRAS Q61H (e.g., LY3537982, JAB-21822, BBO-8520, D-1553, BI-1823911, RMC-9805, MRTX1133, MRTX849, AMG510, GDC-6036, AZD4625, JDQ443, RMC-6291, RMC-6236, BI-2493, MK-1084, RMC-8839, SHR1127, JAB-21822, GFH925, IBI351, BPI-421286, JMKX1899, HBI-2438, D3S-001, GFH375, VS-7375, RMC-7977, RMC-5127, FMC-376), and (3) a MET inhibitor (e.g., foretinib, AMG-458, tivantinib, crizotinib, cabozantinib, tepotinib, capmatinib, savolitinib, glesatinib).

The practice of any treatment methods disclosed herein may involve administering a SOS1 inhibitor in combination or in conjunction with an additional therapy. Applicable therapies for treating Ph+ diseases (including Ph+ blood cancers) include surgery, radiotherapy, cell therapy, chemotherapy, bone marrow transplant, and radiation.

In some embodiments, a subject SOS1 inhibitor is administered as a monotherapy to a subject in need thereof. As used herein, the term “monotherapy” means that only one therapy for the intended purpose (e.g., treating cancer, or specifically for treating Ph+ cancers) is administered to the subject during the treatment cycle. In some embodiments, the SOS1 inhibitor is administered without a TKI. In some embodiments, the SOS1 inhibitor is administered without an anti-cancer agent including chemotherapeutic agents. In some embodiments, other therapies, however, can be administered to the subject for other purposes. For example, anti-inflammatory agents or other agents that treat symptoms associated with Ph+ cancer, but not the underlying cancer itself, including, e.g., inflammation, pain, nausea, weight loss, and general malaise can be administered to a subject with cancer during the period of monotherapy treatment. A subject being treated by the present methods can have completed any prior treatment (e.g., surgery, chemotherapy, bone marrow transplant, or being treated with some other anti-cancer agents) before the monotherapy treatment or receive additional therapies after the monotherapy treatment. In one aspect, the present disclosure provides a method of treating Philadelphia chromosome-positive (Ph+) acute lymphoblastic leukemia (Ph+ALL) in a subject in need thereof, comprising administering a pharmaceutical composition comprising an effective amount of a small molecule SOS1 inhibitor.

In another aspect, the present disclosure provides a method of combination therapy, comprising administering (a) a small molecule SOS1 inhibitor and (b) a tyrosine kinase inhibitor against BCR-ABL tyrosine kinase (TKI) to a subject in need thereof, wherein the administering of (a) takes places prior to, concurrent with, or subsequent to administering (b), and wherein the combination therapy synergistically reduces progression of a Philadelphia chromosome-positive (Ph+) cancer (e.g., blood cancer), and/or reduces undesirable side effects associated with the TKI. The combination therapy can be applied to treat chronic myelogenous leukemia (CML), Ph+ acute lymphoblastic leukemia (Ph+ALL), and Ph+ lymphoblastic lymphoma (Ph+LBL).

A synergistic effect of a combination treatment comprising a plurality of agents, as disclosed herein, can be characterized by a synergistic value as ascertained by an “excess over Bliss independence” or “BLISS” independence criterion. The BLISS independence criterion can be used to screen for candidate drug combinations. The criterion can compare the observed combination response with the predicted combination response, which predicted combination response is obtained based on the assumption that there is no effect from drug-drug interactions. The combination effect can be determined to be synergistic when the observed combination response is greater than the predicted combination response (e.g., greater by a threshold value). To determine a synergistic value of a combination treatment comprising inhibitor (a) (e.g., a SOS1 inhibitor) and inhibitor (b) (e.g., a TKI disclosed or exemplified herein) for inducing growth inhibition of target cells (e.g., Ph+ cells), the BLISS independence criterion can utilize the following equation:

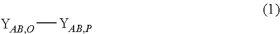

wherein:
Y_{AB,O} is observed percentage growth inhibition of the target cells by the combination comprising (a) at dose A and (b) at dose B; and
Y_{AB,P} is predicted percentage growth inhibition of the target cells by the combination comprising (a) at the dose A, and (b) at the dose B, wherein:

$${Y}_{A B , P =} {Y}_{A} + {Y}_{B} - {Y}_{A} {Y}_{B} \qquad ( 2 )$$

wherein further:
Y_{A} is observed percentage growth inhibition of the target cells by (a) alone at the dose A;
Y_{B} is observed percentage growth inhibition of the target cells by (b) alone at the dose B; and Y_{A}Y_{B} is product of Y_{A} and Y_{B}.
   The observed combined percentage inhibition Y_{AB,O} is compared with the predicted percentage growth inhibition Y_{AB,P} in accordance with equation (1). The comparison can determine whether the combination treatment promotes a synergistic effect, an additive effect, or an antagonistic effect, as summarized in equation (3). When Y_{AB,O}>Y_{AB,P}, the combination treatment can be determined to be more efficacious than expected (e.g., a synergistic effect). When Y_{AB,O}<Y_{AB,P}, the combination treatment can be determined to be worse than expected (e.g., an antagonistic effect). When Y_{AB,O}=Y_{AB,P}, the combination treatment can be determined to be substantially the same as a simple addition of two separate drugs (e.g., independent effects, or an additive effect).

$${Y}_{A B , P} \{ \begin{matrix}> {Y}_{AB , P} & Synergy \\ = {Y}_{AB , P} & Independent \\ < {Y}_{AB , P} & Antagonism\end{matrix} \qquad ( 3 )$$

When using the BLISS independence criterion, the percentage growth inhibition of the target cells can be provided based on a percentage scale (e.g., between about 0% to about 100%) or a fractional scale (e.g., between about 0 to 1). For example, a 75% growth inhibition of the target cells can be expressed as 0.75 for purposes of analysis in accordance with the BLISS independence criterion. For example, with the fractional scale is used, the difference between the observed combined percentage inhibition Y_{AB,O} and the predicted percentage growth inhibition Y_{AB,P}, in accordance with equation (1) (e.g., based on one or more in vitro experiments), can be determined to be additive (or antagonistic) when the difference is less than or equal to zero. Such difference can be determined to be synergistic when the difference is greater than zero. Here, the synergistic effect can be divided into a plurality of sub-ranges, e.g., a first synergistic sub-range having the difference between about 0.05 and about 0.1 (e.g., mild synergy), a second synergistic sub-range having the difference between about 0.1 and about 0.2 (e.g., moderate synergy), and a third synergistic sub-range having the difference greater than or equal to 0.2 (e.g., robust synergy).

In some embodiments, the combination treatment comprising a plurality of agents (e.g., a SOS1 inhibitor and a TKI disclosed or exemplified herein), can be utilized to reduce growth or proliferation of target cells, such as Ph+ cells, in vitro or in vivo. The therapeutic efficacy of the combination treatment can be characterized by a synergistic value of above 0.1 as ascertained by the Bliss independent criterion. The therapeutic efficacy of the combination treatment can be characterized by a synergistic value of at least about 0.1, 0.2, 0.3, 0.4, 0.5, 1, 1.5, 2, 2.5, 3, 3.5, 4, 4.5, 5, 5.5, 6, 6.5, 7, 7.5, 8, 9, 10, 11, 12, 13, 14, 15, or above as ascertained by the Bliss independent criterion. The therapeutic efficacy of the combination treatment can be characterized by a synergistic value of about 0.1 to about 1, about 1.0-5, about 5 to about 10, or about 10-15.

The subject combination therapy comprising a plurality of different therapeutic agents (e.g. a SOS1 inhibitor and a TKI) can synergistically achieve one or more desired therapeutic actions or outcomes, including, but not limited to, reduced progression of chronic myelogenous leukemia (CML), Ph+ acute lymphoblastic leukemia (Ph+ALL), or Ph+ lymphoblastic lymphoma (Ph+LBL). In some embodiments, the reduced progression of a Philadelphia chromosome-positive (Ph+) blood cancer is evidenced by an improvement of a response such as hematological response, molecular response, and cytogenetic response in a subject being treated. In some embodiments, an improvement of hematological response is characterized by blood cells of the subject that return to or partially return to normal levels. In some embodiments, an improvement of hematological response is characterized by one or more of <10×10⁹/L white blood cell count, <450×10⁹/L platelet count, <5% myelocytes plus metamyelocytes in peripheral blood, <20% basophils in peripheral blood, no blasts or promyelocytes in peripheral blood, no extramedullary involvement, no immature granulocytes in differentiation, and nonpalpable spleen. In some embodiments, an improvement of hematological response is characterized by <10% blasts in peripheral blood, such as <9%, <8%, <7%, <6%, <5%, <4%, <3%, <2%, or <1% blasts in peripheral blood. In some embodiments, an improvement of molecular response is characterized by a reduction in amount of BCR-ABL1 gene present in the subject's blood as ascertained by a nucleic acid assay. In some embodiments, an improvement of molecular response is characterized by a reduction in amount of BCR-ABL1 gene expression in the subject's blood as ascertained by a protein assay. In some embodiments, an improvement of molecular response is characterized by a BCR-ABL1 level less than 10%, such as less than 9%, less than 8%, less than 7%, less than 6%, less than 5%, less than 4%, less than 3%, less than 2%, less than 1%, less than 0.1%, less than 0.01%, or less than 0.0032%. In some embodiments, an improvement of molecular response is characterized by at least a 3-log reduction in the level of BCR-ABL1 gene present in the subject's blood, such as at least a 4-log, 4.5-log, or 5-log reduction in the level of BCR-ABL1 gene present in the subject's blood, optionally as ascertained by a qPCR assay. In some embodiments, an improvement of cytogenetic response is characterized by a reduction in number of blood cells comprising Philadelphia chromosome as ascertained by, e.g., FISH, PCR, sequencing, or other techniques. In some embodiments, an improvement of cytogenetic response is characterized by a presence of <40% Ph+ metaphases in at least 20 metaphases, such as <35% Ph+ metaphases, <30% Ph+ metaphases, <25% Ph+ metaphases, <20% Ph+ metaphases, <15% Ph+ metaphases, <10% Ph+ metaphases, <5% Ph+ metaphases, or no detectable (0%) Ph+ metaphases in at least 20 metaphases. In some embodiments, an improvement of cytogenetic response is characterized by less than 40% of cells comprising the Ph chromosome, such as less than 35%, less than 30%, less than 25%, less than 20%, less than 15%, less than 10%, less than 5%, or no detectable cells comprising the Ph chromosome.

A hematological response, molecular response, and/or cytogenetic response of a subject to a method of treatment described herein may persist for at least 1 month, such as at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. For example, a subject treated according to a method of the present disclosure may exhibit a hematological response characterized by one or more of (i)<10×10⁹/L white blood cell count, (ii)<450×10⁹/L platelet count, (iii) no immature granulocytes in differentiation, (iv) nonpalpable spleen, and (v)<10% blasts in peripheral blood, any one or more of which may persist for at least 1 month, such as at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. In some embodiments, a subject treated according to a method of the present disclosure exhibits a molecular response characterized by (i) a BCR-ABL1 level less than 10%, such as less than 9%, less than 8%, less than 7%, less than 6%, less than 5%, less than 4%, less than 3%, less than 2%, or less than 1%, or (ii) at least a 3-log reduction in the level of BCR-ABL1 gene present in the subject's blood, such as at least a 4-log, 4.5-log, or 5-log reduction in the level of BCR-ABL1 gene present in the subject's blood, optionally as ascertained by a qPCR assay, either or both of which persists for at least 1 month, such as at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer. In some embodiments, a subject treated according to a method of the present disclosure exhibits a cytogenetic response characterized by (i) a presence of <40% Ph+ metaphases in at least 20 metaphases, such as <35% Ph+ metaphases, <30% Ph+ metaphases, <25% Ph+ metaphases, <20% Ph+ metaphases, <15% Ph+ metaphases, <10% Ph+ metaphases, <5% Ph+ metaphases, or no detectable (0%) Ph+ metaphases in at least 20 metaphases, or (ii) less than 40% of cells comprising the Ph chromosome, such as less than 35%, less than 30%, less than 25%, less than 20%, less than 15%, less than 10%, less than 5%, or no detectable cells comprising the Ph chromosome, either or both of which persists for at least 1 month, such as at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or longer.

The subject combination treatment disclosed herein can achieve such desired actions or outcomes, while synergistically providing one or more superior advantages including, but not limited to, (i) decreased amount (e.g., dosage regimen, number of doses, etc.) of one, more, or all of the therapeutic agents utilized in the combination therapy; (ii) avoiding, limiting, or reducing one or more undesirable side-effects associated with the use of any one of the plurality of different therapeutic agents when used in the therapeutically effective amount or clinically approved amount; (iii) allowing for the use of a TKI that would otherwise be intolerable to the subject.

Through the administration of a combination therapy disclosed herein, a range of undesirable side-effects associated may be reduced, including cardio-toxicity, nausea, vomiting, diarrhea, muscle cramp, bone pain, fatigue, rashes, and edema. In some embodiments, the cardiovascular side effects ranging from hypertension, atrial fibrillation, reduced cardiac function, heart failure, and sudden death can be reduced. In some embodiments, other side effects of a TKI including nausea, vomiting and/or diarrhea, muscle cramps and bone pain, fatigue, rashes, and edema can be reduced. The combination treatment with a SOS1 inhibitor disclosed herein may allow administration of a TKI that is otherwise not tolerable to the subject. The combination treatment with a subject SOS1 inhibitor may allow a more frequent dosage regimen of TKI that is otherwise too toxic or not tolerable to a subject in need of such treatment.

The combination treatment as disclosed herein can utilize a TKI that can be therapeutically sub-optimal when used alone, but in combination with a SOS1 inhibitor disclosed herein yields an overall therapeutic efficacy (e.g., promoting one or more desired therapeutic outcomes and/or reducing undesirable side-effects). In some embodiments, the combination therapy utilizes a sub-therapeutic dose of a TKI approved for treating a human subject for a given indication. In some embodiments, the sub-therapeutic dose is less than 95% of the approved dose of the TKI, such as less than 90%, less than 80%, less than 70%, less than 60%, less than 50%, less than 40%, less than 30%, less than 20%, or less than 10% of the approved dose of the TKI.

For example, a sub-therapeutic dose of dasatinib is less than the approved dose of 100 mg daily for treating chronic phase CML or less than the approved dose of 140 mg daily for treating accelerated and blast phase CML and Ph+ ALL. For example, a sub-therapeutic dose of imatinib is less than the approved dose of 400 mg daily for treating chronic phase CML or less than the approved dose of 600 mg daily for treating accelerated and blast phase CML and Ph+ ALL. A sub-therapeutic dose of nilotinib is less than the approved dose of 300 mg twice daily for newly-diagnosed chronic phase CML or less than the approved dose of 400 mg twice a day for resistant (after initial TKI treatment) and accelerated or blast phase CML. A sub-therapeutic dose of asciminib for a human subject is less than 80 mg daily or less than 40 mg twice daily for treating CML and Ph+ALL, or less than 200 mg twice a day for Ph+ blood cancer having a T315I mutation. A sub-therapeutic dose of ponatinib is less than 45 mg daily for treating CML and Ph+ALL.

A sub-therapeutic dose of dasatinib may be less than 90% of the approved dose of 100 mg daily for treating chronic phase CML or less than 90% of the approved dose of 140 mg daily for treating accelerated and blast phase CML and Ph+ ALL, such as less than 90 mg daily for treating chronic phase CML or less than 126 mg daily for treating accelerated and blast phase CML and Ph+ ALL. A sub-therapeutic dose of imatinib may be less than 90% of the approved dose of 400 mg daily for treating chronic phase CML or less than 90% of the approved dose of 600 mg daily for treating accelerated and blast phase CML and Ph+ ALL, such as less than 360 mg daily for treating chronic phase CML or less than 540 mg daily for treating accelerated and blast phase CML and Ph+ ALL. A sub-therapeutic dose of nilotinib may be less than 90% of the approved dose of 300 mg twice daily for newly-diagnosed chronic phase CML or less than 90% of the approved dose of 400 mg twice a day for resistant (after initial TKI treatment) and accelerated or blast phase CML, such as less than 270 mg twice daily for newly-diagnosed chronic phase CML or less than 360 mg twice a day for resistant (after initial TKI treatment) and accelerated or blast phase CML. A sub-therapeutic dose of asciminib may be less than 90% of 80 mg daily or less than 40 mg twice daily for treating CML and Ph+ ALL, or less than 90% of 200 mg twice a day for Ph+ blood cancer having a T315I mutation, such as less than 72 mg daily or less than 36 mg twice daily for treating CML and Ph+ALL, or less than 180 mg twice a day for Ph+ blood cancer having a T315I mutation. A sub-therapeutic dose of ponatinib may be less than 90% of 45 mg daily for treating CML and Ph+ALL, such as less than 40.5 mg daily for treating CML and Ph+ALL.

In some embodiments, dasatinib is administered at a dose of 100 mg daily in combination with a SOS1 inhibitor disclosed herein. In some embodiments, dasatinib is administered at a dose of 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, or 240 mg daily in combination with a SOS1 inhibitor disclosed herein.

In practicing any of the subject methods, the SOS1 inhibitor may be administered at a dose of at least 5 mg daily, such as 5 mg, 10 mg, 20 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240 mg, 260 mg, 280 mg, 300 mg, 320 mg, 340 mg, 360 mg, 380 mg, 400 mg, 420 mg, 440 mg, 460 mg, 480 mg, 500 mg, 550 mg, 600 mg, 650 mg, 700 mg, 750 mg, 800 mg, 850 mg, 900 mg, 950 mg, or 1000 mg daily, optionally in combination with a TKI (e.g., dasatinib). In some embodiments, the SOS1 inhibitor is administered at a dose of 5 to 1000 mg, such as about 5 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 550 mg, about 600 mg, about 650 mg, about 700 mg, about 750 mg, about 800 mg, about 850 mg, about 900 mg, about 950 mg, or about 1000 mg, optionally in combination with a TKI, such as in combination with 100 mg dasatinib. In some embodiments, the SOS1 inhibitor is administered once daily in an oral dosage form.

In another aspect, the present disclosure provides a method of treating Philadelphia chromosome-positive (Ph+) blood cancer in a subject in need thereof. The method comprises administering a pharmaceutical composition that comprises an effective amount of a small molecule SOS1 inhibitor to the subject, wherein said SOS1 inhibitor inhibits growth of a CML cell line with an IC50 less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, 0.1 nM or even less, as ascertained in a growth inhibition assay utilizing the CML cell line. In some embodiments, the SOS1 inhibitor inhibits a CML cell line selected from the group consisting of K562, BV173, KCL22-s, KCL22-imatinib-resistant cell line, Ba/F3 (BRC-ABL), BA/F3 (BCR-ABL with T315I mutation), Ba/F3 (BCR-ABL with F359V mutation), with an IC50 less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, 0.1 nM or even less. In some embodiments, the SOS1 inhibitor inhibits growth of a CML cell line with an IC50 at least 100 times less than that of BI3406 or that of MRTX0902. In some embodiments, the SOS1 inhibitor synergistically inhibits growth of a CML cell line in combination with a tyrosine kinase inhibitor against BCR-ABL tyrosine kinase (TKI). Exemplary TKIs for use in such combinations can be one or more of imatinib, dasatinib, nilotinib, bosutinib, radotinib, flunatinib, ponatinib, olverembatinib, and asciminib. In some embodiments, the SOS1 inhibitor is characterized in that it synergistically inhibits growth of CML cells in combination with a TKI to yield at least about 80% growth inhibition, when less than about 50 nM, 40 nM, 30 nM, 20 nM, 10 nM, 5 nM, 4 nM, 3 nM, 2 nM, 1 nM, 0.5 nM, 0.4 nM, 0.3 nM, 0.2 nM, or 0.1 nM of the SOS1 inhibitor is applied in combination with the TKI applied at its IC50 molarity, in an in vitro growth inhibition assay using CML cells. In some embodiments, the SOS1 inhibitor is characterized in that it synergistically increases percent of cell growth inhibition from about 10% to at least about 50%, when less than about 50 nM, 40 nM, 30 nM, 20 nM, 10 nM, 5 nM, 4 nM, 3 nM, 2 nM, 1 nM of the SOS1 inhibitor is applied in combination with the TKI applied at its IC10 molarity, as ascertained in an in vitro growth inhibition assay using CML cells. In some embodiments, the SOS1 inhibitor is characterized in that it synergistically increases percent of cell growth inhibition from about 50% to at least about 80%, when less than about 50 nM, 40 nM, 30 nM, 20 nM, 10 nM, 5 nM, 4 nM, 3 nM, 2 nM, 1 nM of the SOS1 inhibitor is applied in combination with the TKI applied at its IC50 molarity, in an in vitro growth inhibition assay using CML cells.

In yet another aspect, the present disclosure provides a method of reducing proliferation of a cell comprising a Philadelphia chromosome. The method comprises administering to the cell (a) a small molecule SOS1 inhibitor disclosed herein, and (b) at least one tyrosine kinase inhibitor against BCR-ABL tyrosine kinase (TKI), wherein the administration of (a) and (b) synergistically inhibits growth of CML cells as evidenced by achieving a comparable or higher degree of growth inhibition when either (1) less than 90% of the SOS1 inhibitor is administered as compared to the amount required for the SOS1 inhibitor when administered alone; or (2) less than 90% of the TKI is administered as compared to the amount required for the TKI when administered alone. In some embodiments, the TKI administered is less than about 80%, 70%, 60%, 50%, 40%, 30%, 25%, 20%, 15%, or 10% of the amount required to achieve a comparable or higher degree of growth inhibition. In some embodiments, the practice of this method of reducing proliferation of Ph+ chronic myelogenous leukemia (CML) cells, Ph+ALL cells, or Ph+LBL is in vitro, ex vivo, or in vivo.

### Kits

Another aspect of the present disclosure provides a kit for use in reducing proliferation of a cell comprising a Philadelphia chromosome. Typically, the kit comprises: (1) a composition comprising a small molecule SOS1 inhibitor of Formula (I), (I-A), (I-B), (I-1), (I-B31), (I-B32), (I-C), (I-C1), (I-C2), (I-C3), (I-D), (I-D1), (I-D2), (I-E), (I-E1), (II-B), (II-C), (III), (A), (A-1), or (Aa-1); and (2) instructions directing contacting the cell with the composition. In some embodiments, the small molecule SOS1 inhibitor is a compound described in Table 1 or Table 2, or a pharmaceutically acceptable salt or solvate thereof. In some embodiments, instructions for contacting the cells in vitro, ex vivo, or in vivo are provided. In some embodiments, the kit further comprises at least one TKI known in the art or disclosed herein, including imatinib, dasatinib, nilotinib, bosutinib, radotinib, flumatinib, ponatinib, olverembatinib, and asciminib. In some embodiments, the SOS1 inhibitor and the at least one TKI are formulated in the same unit dosage form. In some embodiments, the SOS1 inhibitor and the at least one TKI are formulated in a different unit dosage form. In some embodiments, the kit can comprise a multi-day supply, including instructions directing the multi-day administration.

### Pharmaceutical Compositions and Methods of Administration

In certain aspects, the present disclosure provides a pharmaceutical composition comprising a compound described herein, or a pharmaceutically acceptable salt or solvate thereof, and a pharmaceutically acceptable excipient.

A compound of Formula (I), (I-A), (I-B), (I-1), (I-B1), (I-B2), (I-C), (I-C1), (I-C2), (I-C3), (I-D), (I-D1), (I-D2), (I-E), (I-E1), (II-B), (II-C), (III), (A), (A-1), or (Aa-1), or a pharmaceutically acceptable salt or solvate thereof, may be administered to a subject in a biologically compatible form suitable for administration to treat or prevent a disease, disorder or condition. A compound described herein may be administered in any pharmacological form including a therapeutically effective amount of a compound of Formula (I), (I-A), (I-B), (I-1), (I-B1), (I-B2), (I-C), (I-C1), (I-C2), (I-C3), (I-D), (I-D1), (I-D2), (I-E), (I-E1), (II-B), (II-C), (III), (A), (A-1), or (Aa-1), or a pharmaceutically acceptable salt or solvate thereof, alone or in combination with a pharmaceutically acceptable carrier. In some embodiments, the present disclosure provides a pharmaceutical composition comprising a pharmaceutically acceptable excipient and a compound of Formula (I), (I-A), (I-B), (I-1), (I-B1), (I-B2), (I-C), (I-C1), (I-C2), (I-C3), (I-D), (I-D1), (I-D2), (I-E), (I-E1), (II-B), (II-C), (III), (A), (A-1), or (Aa-1), or a pharmaceutically acceptable salt or solvate thereof.

In some embodiments, a compound described herein is administered as a pure chemical. In some embodiments, a compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient of the composition.

In practicing any of the subject methods, the compounds described herein may be administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon, for example, the condition and disorder of the recipient. By way of example only, a compound described herein can be administered locally to the area in need of treatment, by for example, local infusion during surgery, topical application such as creams or ointments, injection, catheter, or implant. The administration can also be by direct injection at the site of a diseased tissue or organ. In some embodiments, a compound described herein is administered orally.

In practicing any of the subject methods, a pharmaceutical composition suitable for oral administration may be presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, a pharmaceutical composition is formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided for illustrative purposes only and not to limit the scope of the claims provided herein.

### Example 1: Ras Sequence

| Human K-Ras4b (SEQ ID NO. 1): | |
|---|---|
| 1 | MTEYKLVVVG AGGVGKSALT IQLIQNHFVD EYDPTIEDSY RKQVVIDGET |
| 51 | CLLDILDTAG QEEYSAMRDQ YMRTGEGFLC VFAINNTKSF EDIHHYREQI |
| 101 | KRVKDSEDVP MVLVGNKCDL PSRTVDTKQA QDLARSYGIP FIETSAKTRQ |
| 151 | GVDDAFYTLV REIRKHKEKM SKDGKKKKKK SKTKCVIM |
| Human SOS1 (SEQ ID NO. 2): | |
| 1 | MQAQQLPYEF FSEENAPKWR GLLVPALKKV QGQVHPTLES NDDALQYVEE |
| 51 | LILQLLNMLC QAQPRSASDV EERVQKSFPH PIDKWAIADA QSAIEKRKRR |
| 101 | NPLSLPVEKI HPLLKEVLGY KIDHQVSVYI VAVLEYISAD ILKLVGNYVR |
| 151 | NIRHYEITKQ DIKVAMCADK VLMDMFHQDV EDINILSLTD EEPSTSGEQT |
| 201 | YYDLVKAFMA EIRQYIRELN LIIKVFREPF VSNSKLFSAN DVENIFSRIV |
| 251 | DIHELSVKLL GHIEDTVEMT DEGSPHPLVG SCFEDLAEEL AFDPYESYAR |
| 301 | DILRPGFHDR FLSQLSKPGA ALYLQSIGEG FKEAVQYVLP RLLLAPVYHC |
| 351 | LHYFELLKQL EEKSEDQEDK ECLKQAITAL LNVQSGMEKI CSKSLAKRRL |
| 401 | SESACRFYSQ QMKGKQLAIK KMNEIQKNID GWEGKDIGQC CNEFIMEGTL |
| 451 | TRVGAKHERH IFLFDGLMIC CKSNHGQPRL PGASNAEYRL KEKFFMRKVQ |
| 501 | INDKDDTNEY KHAFEIILKD ENSVIFSAKS AEEKNNWMAA LISLQYRSTL |
| 551 | ERMLDVTMLQ EEKEEQMRLP SADVYRFAEP DSEENIIFEE NMQPKAGIPI |
| 601 | IKAGTVIKLI ERLTYHMYAD PNFVRTFLTT YRSFCKPQEL LSLIIERFEI |
| 651 | PEPEPTEADR IAIENGDQPL SAELKRFRKE YIQPVQLRVL NVCRHWVEHH |
| 701 | FYDFERDAYL LQRMEEFIGT VRGKAMKKWV ESITKIIQRK KIARDNGPGH |
| 751 | NITFQSSPPT VEWHISRPGH IETFDLLTLH PIEIARQLTL LESDLYRAVQ |
| 801 | PSELVGSVWT KEDKEINSPN LLKMIRHTTN LTLWFEKCIV ETENLEERVA |
| 851 | VVSRIIEILQ VFQELNNFNG VLEVVSAMNS SPVYRLDHTF EQIPSRQKKI |
| 901 | LEEAHELSED HYKKYLAKLR SINPPCVPFF GIYLTNILKT EEGNPEVLKR |
| 951 | HGKELINFSK RRKVAEITGE IQQYQNQPYC LRVESDIKRF FENLNPMGNS |
| 1001 | MEKEFTDYLF NKSLEIEPRN PKPLPRFPKK YSYPLKSPGV RPSNPRPGTM |
| 1051 | RHPTPLQQEP RKISYSRIPE SETESTASAP NSPRTPLTPP PASGASSTTD |
| 1101 | VCSVFDSDHS SPFHSSNDTV FIQVTLPHGP RSASVSSISL TKGTDEVPVP |
| 1151 | PPVPPRRRPE SAPAESSPSK IMSKHLDSPP AIPPRQPTSK AYSPRYSISD |
| 1201 | RTSISDPPES PPLLPPREPV RTPDVFSSSP LHLQPPPLGK KSDHGNAFFP |
| 1251 | NSPSPFTPPP PQTPSPHGTR RHLPSPPLTQ EVDLHSIAGP PVPPRQSTSQ |
| 1301 | HIPKLPPKTY KREHTHPSMH RDGPPLLENA HSS |

### Example 2: SOS Purification

A SOS construct or a variant thereof is His-tagged. E. coli cultures are induced in a fermenter, harvested, and lysed in lysis buffer, for example, in 25 mM Tris HCl 7.5, 500 mM NaCl, 20 mM Imidazol, Complete EDTA-free (Roche)). For immobilized metal ion affinity chromatography (IMAC), the centrifuged lysate (50,000×g, 45 min, 40) is incubated with 30 mL Ni-NTA (Macherey-Nagel; #745400.100) in a spinner flask (16 h, 40) and subsequently transferred to a chromatography column connected to a chromatography system, e.g., an Akta chromatography system. The column is rinsed with wash buffer, e.g., in 25 mM Tris HCl 7.5, 500 mM NaCl, 20 mM Imidazol and the bound protein is eluted with a linear gradient (0-100%) of elution buffer (25 mM Tris HCl 7.5, 500 mM NaCl, 300 mM Imidazol). The main fractions of the elution peak (monitored by OD280) containing homogenous His10-hSOS is pooled.

### Example 3: Ras-SOS Interaction Assay

The ability of any compound of the present disclosure to reduce a Ras protein signaling output by, e.g., interfering or disrupting interaction (or binding) between SOS1 and a Ras protein can be assessed in vitro. For example, the equilibrium interaction of human SOS1 (hSOS1) with human wildtype Kras or K-Ras mutant (e.g., hK-Ras G12C mutant, or hK-Ras G12C) can be assessed as a proxy or an indication for a subject compound's ability to inhibit SOS. Detection of such interaction is achieved by measuring homogenous time-resolved fluorescence resonance energy transfer (HTRF) from (i) a fluorescence resonance energy transfer (FRET) donor (e.g., antiGST-Europium) that is bound to GST-tagged K-Ras G12C to (ii) a FRET acceptor (e.g., anti-6His-XL665) bound to a His-tagged hSOS1.

The assay buffer can contain 5 mM HEPES pH 7.4, 150 mM NaCl, 10 mM EDTA, 1 mM DTT, 0.05% BSA, 0.0025% (v/v) Igepal and 100 mM KF. A Ras working solution is prepared in assay buffer containing typically 10 nM of the protein construct (e.g., GST-tagged hK-Ras G12C) and 2 nM of the FRET donor (e.g., antiGST-Eu(K) from Cisbio, France). A SOS1 working solution is prepared in assay buffer containing typically 10 nM of the protein construct (e.g., His-hSOS1) and 10 nM of the FRET acceptor (e.g., anti-6His-XL665 from Cisbio, France). An inhibitor control solution is prepared in assay buffer containing 10 nM of the FRET acceptor without the SOS1 protein.

A fixed reaction mixture with or without test compound is transferred into a 384-well plate. Ras working solution is added to all wells of the test plate. SOS1 working solution is added to all wells except for those that are subsequently filled with the inhibitor control solution. After approximately 60 min incubation, the fluorescence is measured with a M1000Pro plate reader (Tecan) using HTRF detection (excitation 337 nm, emission 1: 620 nm, emission 2: 665 nm). Compounds are tested in duplicate at different concentrations (for example, 10 μM, 2.5 μM, 0.63 μM, 0.16 μM, 0.04 μM, 0.01 μM test compound). The ratiometric data (i.e., emission 2 divided by emission 1) is used to calculate IC50 values against SOS1 using GraphPad Prism (GraphPad software).

### Example 4: Ras-SOS Cellular Growth Inhibition Assay

The ability of any compound of the present disclosure to inhibit SOS1-mediated signaling and hence Ras protein signaling can be demonstrated by inhibiting growth of a given Kras mutant cell line.

Growth of cells with K-Ras G12C mutation: MIA PaCa-2 (ATCC CRL-1420) and NCI-H1792 (ATCC CRL-5895) cell lines comprise a G12C mutation and can be used to assess Ras cellular signaling in vitro, e.g., in response to a subject compound of the present disclosure. This cellular assay can also be used to discern selective inhibition of subject compounds against certain types of Kras mutants, e.g., more potent inhibition against Kras G12D relative to Kras G12C mutant, by using MIA PaCa-2 (G12C driven tumor cell line) as a comparison. Cell culture medium (comprising, for example, MIA PaCa-2 cells) is prepared with DMEM/Ham's F12 (e.g., with stable Glutamine, 10% FCS, and 2.5% Horse Serum). NCI-H1792 culture medium is prepared with RPMI 1640 (e.g., with stable Glutamine) and 10% FCS. A CellTiter-Glo (CTG) luminescent based assay (Promega) is used to assess growth of the cells, as a measurement of the ability of the compounds herein to inhibit Ras signaling in the cells. The cells (e.g., 800-1200 per well) are seeded in their respective culture medium in standard tissue culture-treated ultra-low attachment surface 96-well format plates (Corning Costar #3474). The day after plating, cells are treated with a dilution series (e.g., a 9 point 3-fold dilution series) of the compounds herein (e.g., approximately 125 μL final volume per well). Cell viability can be monitored (e.g., approximately 5 days later) according to the manufacturer's recommended instructions, where the CellTiter-Glo reagent is added (e.g., approximately 65 μL), vigorously mixed, covered, and placed on a plate shaker (e.g., approximately for 20 min) to ensure sufficient cell lysis prior to assessment of luminescent signal. The IC50 values are determined using the four-parameter fit. The resulting IC50 value is a measurement of the ability of the compounds herein to reduce cell growth of Ras-driven cells as representative tumor cells.

### Example 5: Inhibition of Ph+ Cell Proliferation Using SOS1 Inhibitors

Ph+ cell lines including CML cell lines such as K562, BV173, KCL22-s, KCL22-r, KCL22-r (imatinib-resistant), Ba/F3-BCR-ABL, Ba/F3-BCR-ABL (with T315I mutation), and Ba/F3-BCR-ABL (with F359V mutation) are commercially available. These cell lines are purchased from the American Type Culture Collection (ATCC) and other sources. Cells are maintained in a humidified incubator at 37° C. with 5% CO₂, and grown in RPMI 1640 with 10% FBS (Gibco) and 50 IU mL⁻¹ penicillin/streptomycin (Gibco). Reference BCR-ABL TKI inhibitors including dasatinib, imatinib, ponatinib, and asciminib are commercially available, for example, from LC Laboratories and MedChemExpress (MCE).

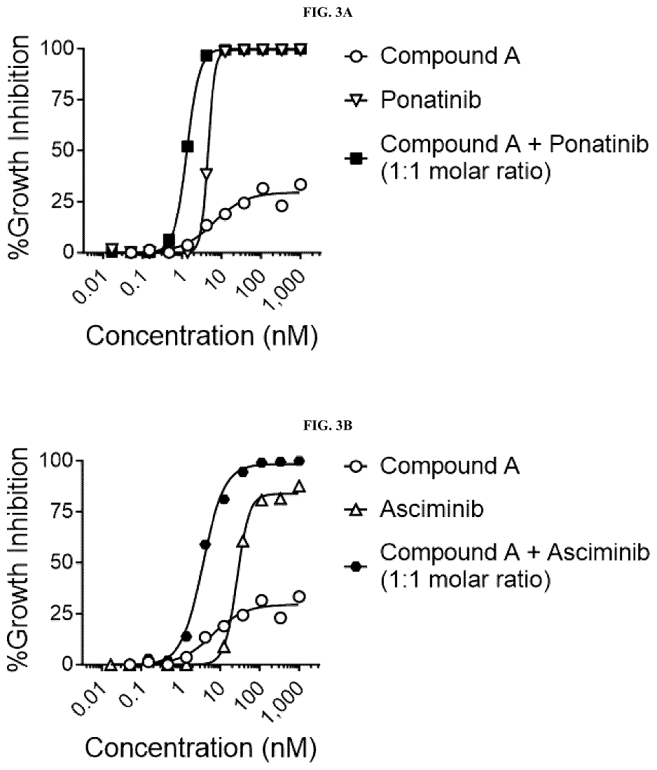

For comparison of anti-growth activity, a CellTiter-Glo (CTG) luminescent based assay (Promega) is used. Cells (~300-1,200 per well) are seeded (using the same media) in standard tissue culture-treated 384-well format plates. The day after plating, cells are treated with a 9 point, 3-fold dilution series of indicated compounds (40 μL final volume per well) and growth inhibition is monitored ~72 hours later according to the manufacturer's recommended instructions, where 20 μL of CellTiter-Glo reagent is added, vigorously mixed, covered, and placed on a plate shaker for 20 min to ensure complete cell lysis prior to assessment of luminescent signal. Growth inhibition of cells treated with a SOS1 inhibitor of the present disclosure in comparison to selected TKIs are monitored as stated above. The experiment demonstrates a concentration dependent growth inhibition of Ph+ cells (across all CML cells tested) by a SOS1 inhibitor disclosed herein. FIG. 3A and FIG. 3B show that a SOS1 inhibitor of the present disclosure (Compound A) in combination with ponatinib or asciminib, respectively, synergistically inhibits cell growth of BCR-ABL transformed mouse cells (Ba/F3-p210-BCR-ABL1-T315I cell line) in a 5-day growth inhibition assay.

### Example 6: Inhibition of Tumor Growth in Ph+ CML Xenograft Model

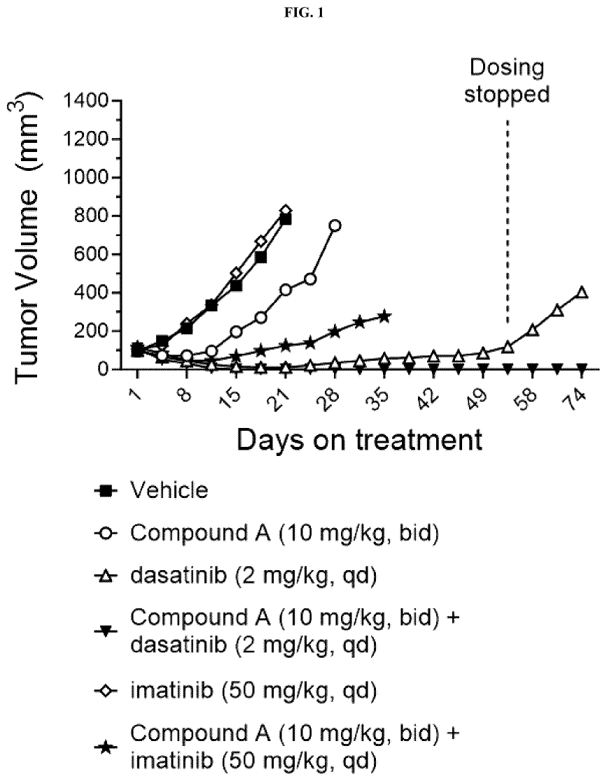

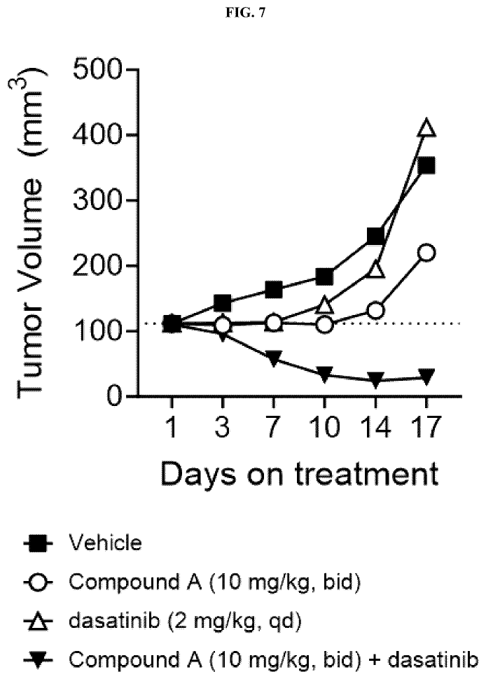

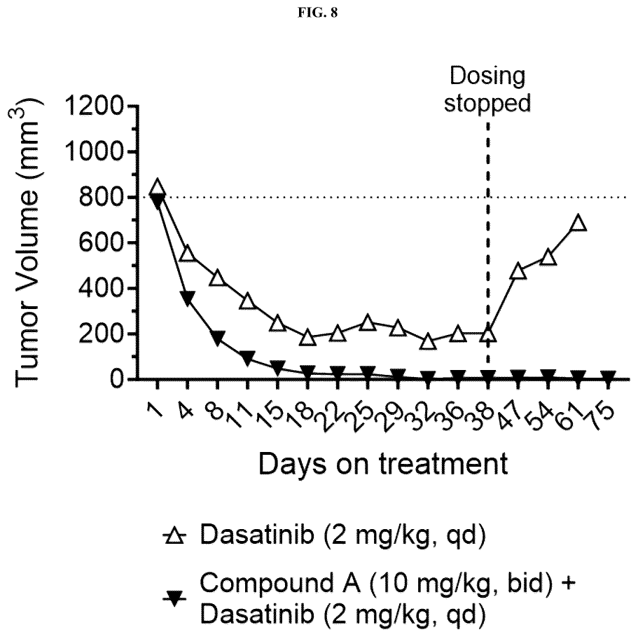

Mice were maintained under specific pathogen-free conditions, and food and water were provided ad libitum. Female 6- to 8-week-old athymic BALB/c nude (NCr) nu/nu mice were used to inoculate Ph+ CML cell lines. For example, KCL22-s cells (5×10⁶) were harvested on the day of use and injected in growth-factor-reduced Matrigel/PBS (50% final concentration in 100 μL). One flank was inoculated subcutaneously per mouse. Mice were monitored daily, weighed twice weekly, and caliper measurements began when tumors became visible. Animals were randomly assigned to treatment groups as indicated in FIG. 1 by an algorithm that assigns animals to groups to achieve best case distributions of mean tumor size with lowest possible standard deviation. Tumor volume was calculated by measuring two perpendicular diameters using the following formula: (L×w²)/2, in which L and w refer to the length and width of the tumor diameter, respectively. When tumors reached an average size of 100 mm³, mice were randomized and treated with vehicle (20% Dexolve) or indicated compounds using a daily or twice daily schedule by oral gavage. Results are expressed as mean and standard deviation of the mean. No apparent toxicity or body weight loss was observed. FIG. 1 shows that a SOS1 inhibitor of the present disclosure (Compound A) alone and in combination with a TKI (e.g., dasatinib or imatinib) is effective in inhibiting tumor growth. Combination treatment with dasatinib renders more than 90% complete responses. Similarly, FIG. 7 shows that a SOS1 inhibitor of the present disclosure (Compound A) is effective in inhibiting tumor growth alone as a monotherapy, and in combination with dasatinib is effective in reducing tumor volume of a dasatinib-refractory tumor (KCL22-r). In a third study, mice were randomized and treated with dasatinib alone or in combination with a SOS1 inhibitor of the present disclosure (Compound A) once KCL-22 tumors reached an average size of 800 mm³ to mimic a remission induction type design. All animals treated with dasatinib in combination with Compound A were rendered tumor free after 38 days of treatment and remained tumor free 3-weeks following cessation of treatment.

### Example 7: Inhibition of Tumor Growth in a Ba/F3 BCR-ABL CML Mouse Allograft Model

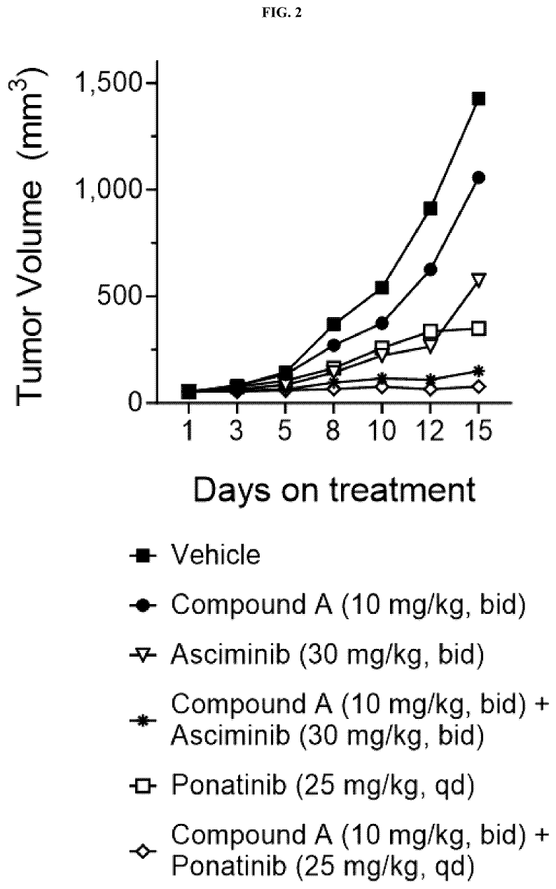

Mice were maintained under specific pathogen-free conditions, and food and water were provided ad libitum. A mouse Ba/F3 cell line that expresses a p210 BCF-ABL1 fusion and T315I mutation was implanted subcutaneously (1×10⁶ cells per mouse) into the right flank of female 6- to 8-week-old NOD/SCID mice. Mice were monitored daily, weighed up to three times weekly, and caliper measurements began when tumors became visible. Animals were randomly assigned to treatment groups as indicated in FIG. 2 by an algorithm that assigns animals to groups to achieve best case distributions of mean tumor size with lowest possible standard deviation. Tumor volume was calculated by measuring two perpendicular diameters using the following formula: (L×w²)/2, in which L and w refer to the length and width of the tumor diameter, respectively. When tumors reached an average size of ~100 mm³, mice were randomized and treated with vehicle, a SOS1 inhibitor of the present disclosure (Compound A), asciminib, ponatinib, Compound A and asciminib, or Compound A and ponatinib by oral gavage at the indicated dose and schedule. No apparent toxicity or body weight loss was observed. FIG. 2 shows that Compound A in combination with a TKI (e.g., asciminib or ponatinib) synergistically reduces tumor volume and extends the duration of controlling tumor growth.

### Example 8: Inhibition of Ph+ ALL Cell Proliferation Using SOS1 Inhibitors

Ph+ cell lines including ALL cell lines such as SUP-B15, Z-33, Z-119, Z-181, SD-1, and TOM-1 are commercially available. These cell lines are purchased from the American Type Culture Collection (ATCC) and other sources. Cells are maintained in a humidified incubator at 37° C. with 5% CO₂, and grown in RPMI 1640 with 10% FBS (Gibco) and 50 IU mL⁻¹ penicillin/streptomycin (Gibco). Reference BCR-ABL TKI inhibitors including dasatinib, imatinib, ponatinib, and asciminib are commercially available, for example, from LC Laboratories and MedChemExpress (MCE).

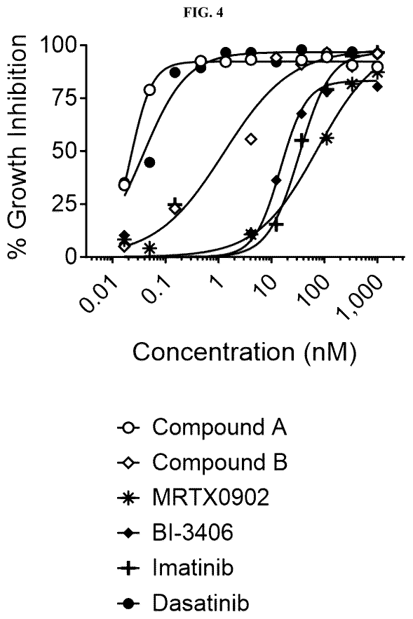

For comparison of anti-growth activity, a CellTiter-Glo (CTG) luminescent based assay (Promega) is used. Cells (~300-1,200 per well) are seeded (using the same media) in standard tissue culture-treated 384-well format plates. The day after plating, cells are treated with a 9 point, 3-fold dilution series of indicated compounds (40 μL final volume per well) and growth inhibition is monitored ~72 hours later according to the manufacturer's recommended instructions, where 20 μL of CellTiter-Glo reagent is added, vigorously mixed, covered, and placed on a plate shaker for 20 min to ensure complete cell lysis prior to assessment of luminescent signal. Growth inhibition of cells treated with a SOS1 inhibitor of the present disclosure in comparison to selected TKIs are monitored as stated above. FIG. 4 shows that SUP-B15 cells, a Ph+ pre-B-ALL cell line, are more sensitive to SOS1 inhibition by compounds of the present disclosure (Compound A, Compound B) compared to other SOS1 inhibitors and TKIs.

### Example 9: Inhibition of Cell Proliferation Using SOS1 Inhibitors in Combination with BCR-ABL TKI Inhibitors

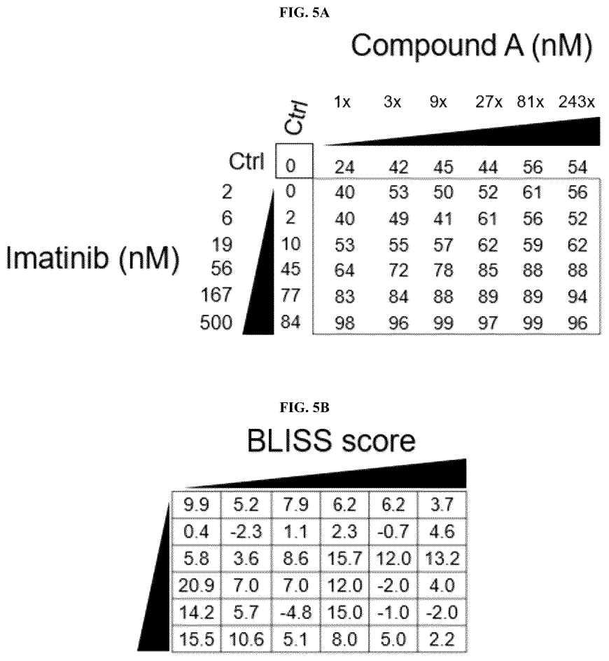

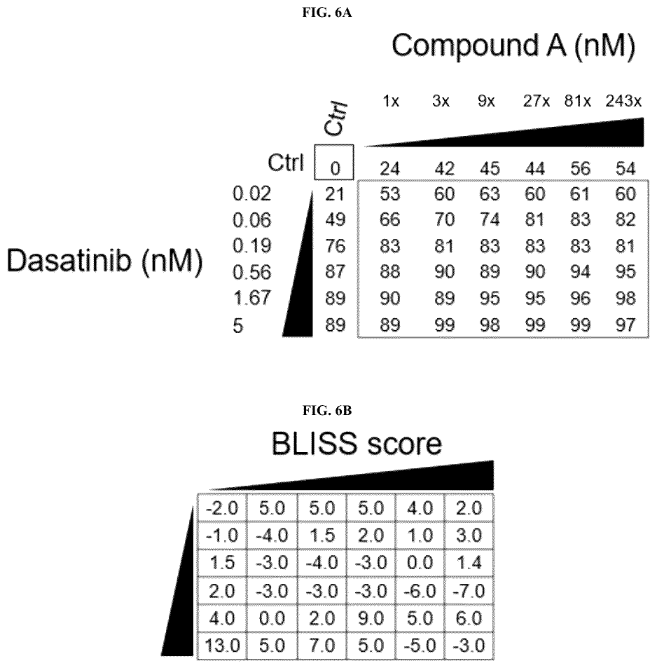

A variety of Philadelphia chromosome-positive (Ph+) or transfected BCR-ABL blood cancer cell lines are commercially available, including K562, BV173, KCL22-s, KCL22-r, KCL22-r (imatinib-resistant), Ba/F3-BCR-ABL, Ba/F3-BCR-ABL (with T315I mutation), Ba/F3-BCR-ABL (with F359V mutation), SUP-B15, Z-33, Z-119, Z-181, SD-1, and TOM-1. These exemplary cell lines can be maintained according to supplier recommendations, such as in a humidified incubator at 37° C. with 5% CO₂ and grown in RPMI 1640 with 10% FBS (Gibco) and 50 IU mL⁻¹ penicillin/streptomycin (Gibco). For comparison of anti-growth activity, a CellTiter-Glo (CTG) luminescent based assay (Promega) is used. Cells (~300-1,200 per well) are seeded (using the same media) in standard tissue culture-treated 384-well format plates. The day after plating, cells are treated with a 9 point, 3-fold dilution series of indicated compounds (40 μL final volume per well) and growth inhibition is monitored ~72 hours later according to the manufacturer's recommended instructions, where 20 μL of CellTiter-Glo reagent is added, vigorously mixed, covered, and placed on a plate shaker for 20 min to ensure complete cell lysis prior to assessment of luminescent signal. Growth inhibition of cells treated with a SOS1 inhibitor of the present disclosure (Compound A) alone and in combination with selected TKIs is monitored as stated above. FIG. 5A and FIG. 5B show that a SOS1 inhibitor of the present disclosure (Compound A) in combination with imatinib synergistically inhibits cell growth of KCL-22 cells. FIG. 6A and FIG. 6B show that a SOS1 inhibitor of the present disclosure (Compound A) in combination with dasatinib synergistically inhibits cell growth of KCL-22 cells.

What is claimed is:

1-36. (canceled)

37. A method of combination therapy, comprising administering (a) a small molecule SOS1 inhibitor and (b) a tyrosine kinase inhibitor against BCR-ABL tyrosine kinase (TKI) to a subject in need thereof, wherein the administering of (a) takes places prior to, concurrent with, or subsequent to administering (b), wherein the combination therapy synergistically reduces progression of a Philadelphia chromosome-positive (Ph+) blood cancer and/or reduces undesirable side effect associated with the TKI, and wherein the SOS1 inhibitor is a compound of Formula (I-1): 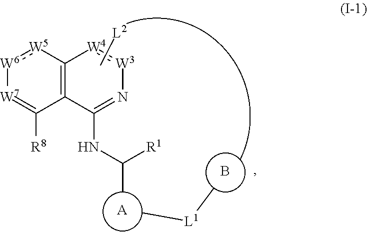
or a pharmaceutically acceptable salt or solvate thereof, wherein: 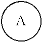
is selected from C₅₋₇ carbocycle and 5- to 7-membered heterocycle, each of which is optionally substituted with one or more R¹¹; 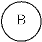
is absent or selected from C₃₋₈ carbocycle and 3- to 8-membered heterocycle, each of which is optionally substituted with one or more R^{11a};
L¹ is selected from a bond, C₁₋₆ alkylene, and C₁₋₆ haloalkylene;
L² is selected from C₅₋₂₅ alkylene, C₅₋₂₅ alkenylene, C₅₋₂₅ alkynylene, 5- to 25-membered heteroalkylene, and 5- to 25-membered heteroalkenylene, each of which is optionally substituted with one or more R^{11b}, wherein L² is covalently bound to one of W³, W⁴, W⁵, W⁶, or W⁷;
W³ is selected from N(R^{3b}), N, C(R³), and C(O);
W⁴ is selected from N(R^{4b}), N, C(R⁴), and C(O);
W⁵ is selected from N(R^{5b}), N, and C(R⁵);
W⁶ is selected from C(R⁶) and C(O);
W⁷ is C(R⁷);
R¹ is C₁₋₃ alkyl optionally substituted with one or more R^{11c};
R⁸ is selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R³, R⁴, R⁵, R⁶, and R⁷ are each independently selected from a bond to L², hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein each C₁₋₆ alkyl, C₂₋₆ alkenyl, C₂₋₆ alkynyl, C₃₋₁₀ carbocycle, and 3- to 10-membered heterocycle is independently optionally substituted with one, two, or three R²⁰;
R^{3b}, R^{4b}, and R^{5b} are each independently selected from a bond to L², hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹⁵, —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵ and —CH₂S(O)₂N(R¹²)(R¹³), wherein each C₁₋₆ alkyl, C₂₋₆ alkenyl, C₂₋₆ alkynyl, C₃₋₁₀ carbocycle and 3- to 10-membered heterocycle is independently optionally substituted with one, two, or three R²⁰;
R¹¹ and R^{11a} are each independently selected at each occurrence from halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11b} is independently selected at each occurrence from halogen, oxo, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, —CH₂S(O)₂N(R¹²)(R¹³), —CH₂N(R¹²)S(O)₂(R¹³), and —P(O)(R¹⁷)(R^{17a}), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R^{11c} is independently selected at each occurrence from halogen, —OR¹², and —N(R¹²)(R¹³);
R¹² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹³ is independently selected at each occurrence from hydrogen, C₁₋₆ alkyl, and C₁₋₆ haloalkyl; or R¹² and R¹³, together with the nitrogen atom to which they are attached, form a 3- to 10-membered heterocycle optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹⁷ and R^{17a} are each independently selected at each occurrence from C₁₋₆ alkyl and C₃₋₆ cycloalkyl, wherein C₁₋₆ alkyl and C₃₋₆ cycloalkyl are optionally substituted with one, two or three R²⁰; or
R¹⁷ and R^{17a} together with the phosphorous atom to which they are attached, form a 3- to 10-membered heterocycle;
R²⁰ is independently selected at each occurrence from halogen, oxo, ═NH, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), —OCH₂C(O)OR²², and —OC(O)R²⁵, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, C₁₋₆ alkyl, C₁₋₆ haloalkyl, C₁₋₆ alkoxy, C₁₋₆ haloalkoxy, —OR²¹, —SR²¹, —N(R²²)(R²³), —C(O)OR²², —C(O)N(R²²)(R²³), —C(O)C(O)N(R²²)(R²³), —OC(O)N(R²²)(R²³), —N(R²⁴)C(O)N(R²²)(R²³), —N(R²⁴)C(O)OR²⁵, —N(R²⁴)C(O)R²⁵, —N(R²⁴)S(O)₂R²⁵, —C(O)R²⁵, —S(O)₂R²⁵, —S(O)₂N(R²²)(R²³), and —OC(O)R²⁵;
R²¹ is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²² is independently selected at each occurrence from H, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ is independently selected at each occurrence from H and C₁₋₆ alkyl;
R²⁴ is independently selected at each occurrence from H and C₁₋₆ alkyl;
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle; and [symbol]
indicates a single or double bond such that all valences are satisfied.

38-43. (canceled)

44. The method of claim 37, wherein the combination therapy exhibits a synergistic effect in reducing side effect associated with the TKI selected from the group consisting of cardio-toxicity, nausea, vomiting, diarrhea, muscle cramp, bone pain, fatigue, rashes, and edema.

45. The method of claim 37, wherein the TKI is dasatinib administered at a sub-therapeutic dose of less than 100 mg daily for treating chronic phase CML or less than 140 mg daily for treating accelerated and blast phase CML or Ph+ALL.

46-49. (canceled)

50. The method of claim 37, wherein Ph+ blood cancer is selected from the group consisting of chronic myelogenous leukemia (CML), Ph+ acute lymphoblastic leukemia (Ph+ALL), and Ph+ lymphoblastic lymphoma (Ph+LBL).

51-63. (canceled)

64. The method of claim 37, wherein the TKI is selected from the group consisting of imatinib, dasatinib, nilotinib, bosutinib, radotinib, flumatinib, ponatinib, olverembatinib, and asciminib.

65. (canceled)

66. (canceled)

67. A method of treating Philadelphia chromosome-positive (Ph+) blood cancer in a subject, the method comprising:
(1) assessing for presence of a genetic aberration associated with resistance to a tyrosine kinase inhibitor against BCR-ABL tyrosine kinase (TKI) in a biological sample that comprises nucleic acid molecules from the subject; and
(2) upon detecting said presence of the genetic aberration, administering to the subject a pharmaceutical composition comprising an effective amount of a small molecule SOS1 inhibitor.

68. (canceled)

69. The method of claim 67, wherein the genetic aberration is a mutation in BCR-ABL selected from the group consisting of T315I, F359V, Y253H, E255K, M351T, G250E, F359I, and H396R.

70. (canceled)

71. (canceled)

72. A method of reducing proliferation of a cell comprising a Philadelphia chromosome, the method comprising administering to the cells (a) a small molecule SOS1 inhibitor and (b) at least one tyrosine kinase inhibitor against BCR-ABL (TKI), wherein the administration of (a) and (b) synergistically inhibits growth of CML cells with a synergistic value of at least about 0.1 as ascertained by Bliss independent criterion.

73. (canceled)

74. (canceled)

75. The method of claim 72, wherein the synergistic value is at least about 1.

76-84. (canceled)

85. The method of claim 37, wherein the compound of Formula (I-1) is a compound of Formula (I-B1) or (I-B2): 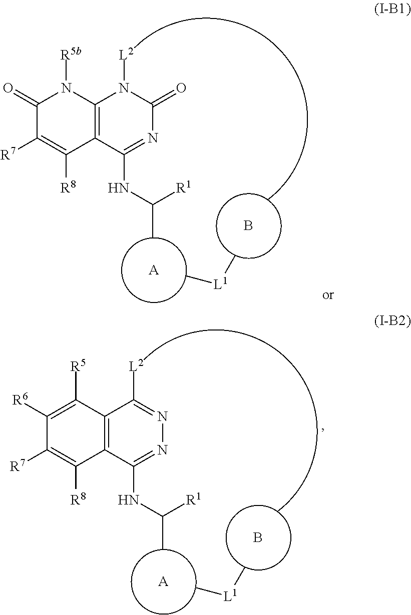
or a pharmaceutically acceptable salt or solvate thereof.

86. The method of claim 37, wherein the compound of Formula (I-1) is a compound of Formula (I-C1), (I-C2), or (I-C3): 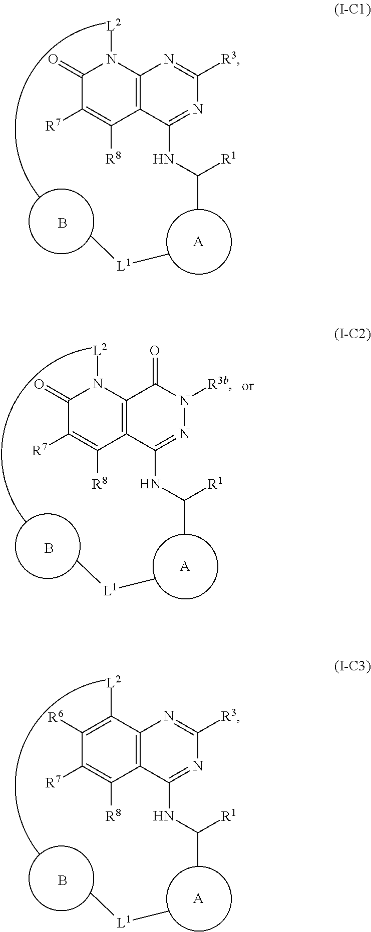
or a pharmaceutically acceptable salt or solvate thereof.

87. The method of claim 37, wherein R¹ is —CH₃, R³ is selected from hydrogen, —CN, —OR¹², and —CH₃, and R⁸ is hydrogen.

88. The method of claim 37, wherein R⁶ is selected from hydrogen, —OR¹², and C₁₋₆ alkyl optionally substituted with one, two, or three R²⁰, and wherein R¹² is selected from C₁₋₆ alkyl.

89. The method of claim 37, wherein R⁷ is selected from C₁₋₆ alkyl, C₃₋₁₀ cycloalkyl, 3- to 10-membered heterocycloalkyl, and —N(R¹²)(R¹³), wherein C₁₋₆ alkyl, C₃₋₁₀ cycloalkyl, and 3- to 10-membered heterocycloalkyl are optionally substituted with one, two, or three R²⁰.

90. The method of claim 37, wherein 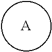
is selected from N 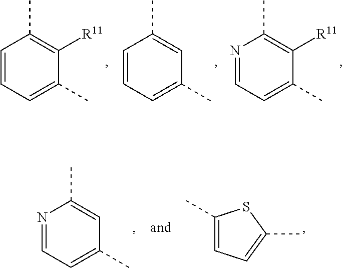
and R¹¹ is independently selected from fluorine and —CH₃.

91. The method of claim 37, wherein L¹ is C₁₋₃ haloalkylene and L² is selected from C₆₋₁₅ alkylene, C₆₋₁₅ alkenylene, C₆₋₁₅ alkynylene, 6- to 15-membered heteroalkylene, and 6- to 15-membered heteroalkenylene, each of which is optionally substituted with one or more R^{11b}.

92. The method of claim 37, wherein 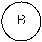
is selected from absent, phenyl, and 4- to 8-membered heterocycle, wherein the phenyl and 4- to 8-membered heterocycle are optionally substituted with one or more R^{11a}.

93. The method of claim 37, wherein the compound of Formula (I-1) is selected from 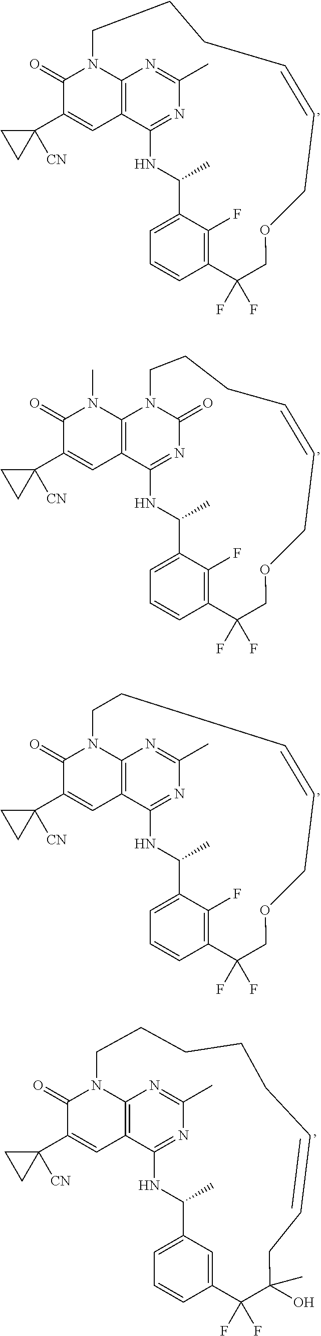 [SMILES: Cc1nc2c3cc(C4(C#N)CC4)c(=O)n(c3n1)CCC/C=C\COCC(F)(F)c1cccc(c1F)[C@@H](C)N2 ; C[C@H]1Nc2nc(=O)n(c3c2cc(C2(C#N)CC2)c(=O)n3C)CCC/C=C\COCC(F)(F)c2cccc1c2F ; Cc1nc2c3cc(C4(C#N)CC4)c(=O)n(c3n1)CC/C=C\COCC(F)(F)c1cccc(c1F)[C@@H](C)N2 ; Cc1nc2c3cc(C4(C#N)CC4)c(=O)n(c3n1)CCCCC/C=C\CC(C)(O)C(F)(F)c1cccc(c1)[C@@H](C)N2] 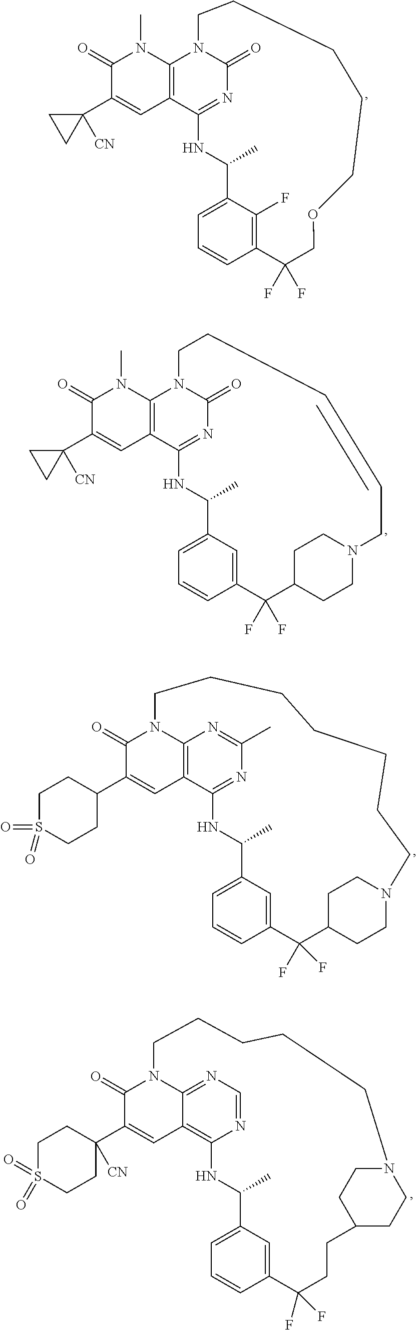 [SMILES: C[C@H]1Nc2nc(=O)n(c3c2cc(C2(C#N)CC2)c(=O)n3C)CCCCCOCC(F)(F)c2cccc1c2F ; C[C@H]1Nc2nc(=O)n(c3c2cc(C2(C#N)CC2)c(=O)n3C)CC/C=C\CN2CCC(CC2)C(F)(F)c2cccc1c2 ; Cc1nc2c3cc(C4CCS(=O)(=O)CC4)c(=O)n(c3n1)CCCCCCCN1CCC(CC1)C(F)(F)c1cccc(c1)[C@@H](C)N2 ; C[C@H]1Nc2ncnc3c2cc(C2(C#N)CCS(=O)(=O)CC2)c(=O)n3CCCCCN2CCC(CC2)CCC(F)(F)c2cccc1c2] 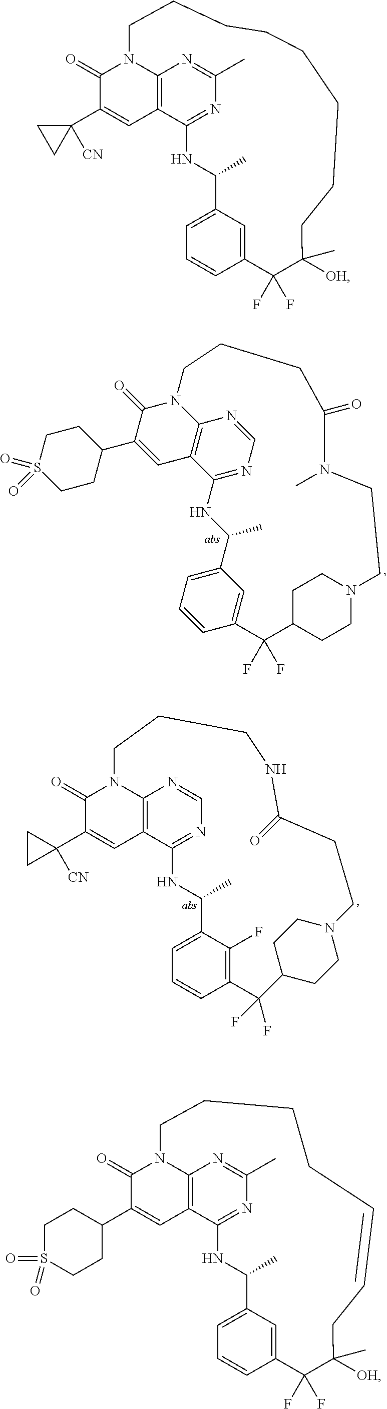 [SMILES: Cc1nc2c3cc(C4(C#N)CC4)c(=O)n(c3n1)CCCCCCCCC(C)(O)C(F)(F)c1cccc(c1)[C@@H](C)N2 ; C[C@H]1Nc2ncnc3c2cc(C2CCS(=O)(=O)CC2)c(=O)n3CCCC(=O)N(C)CCN2CCC(CC2)C(F)(F)c2cccc1c2 ; C[C@H]1Nc2ncnc3c2cc(C2(C#N)CC2)c(=O)n3CCCNC(=O)CCN2CCC(CC2)C(F)(F)c2cccc1c2F ; Cc1nc2c3cc(C4CCS(=O)(=O)CC4)c(=O)n(c3n1)CCCC/C=C\CC(C)(O)C(F)(F)c1cccc(c1)[C@@H](C)N2] 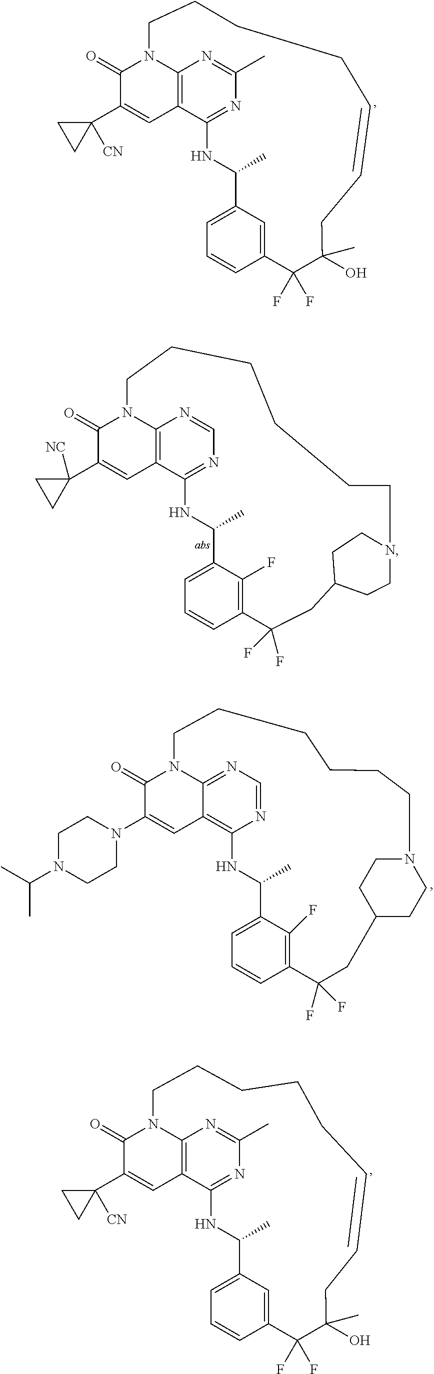 [SMILES: Cc1nc2c3cc(C4(C#N)CC4)c(=O)n(c3n1)CCCC/C=C\CC(C)(O)C(F)(F)c1cccc(c1)[C@@H](C)N2 ; C[C@H]1Nc2ncnc3c2cc(C2(C#N)CC2)c(=O)n3CCCCCCN2CCC(CC2)CC(F)(F)c2cccc1c2F ; CC(C)N1CCN(c2cc3c4ncnc3n(c2=O)CCCCCCN2CCC(CC2)CC(F)(F)c2cccc(c2F)[C@@H](C)N4)CC1 ; Cc1nc2c3cc(C4(C#N)CC4)c(=O)n(c3n1)CCCCC/C=C\CC(C)(O)C(F)(F)c1cccc(c1)[C@@H](C)N2] 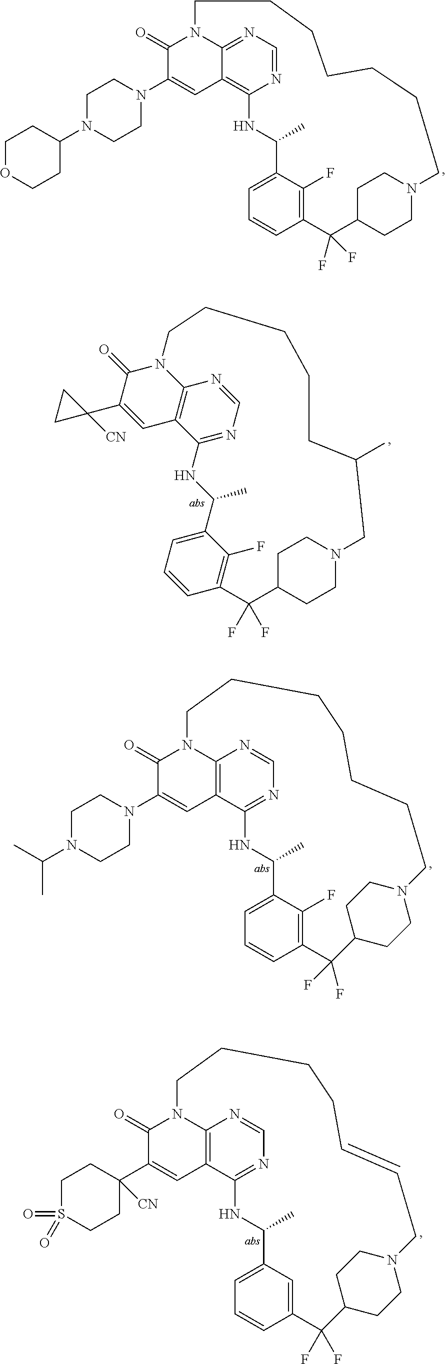 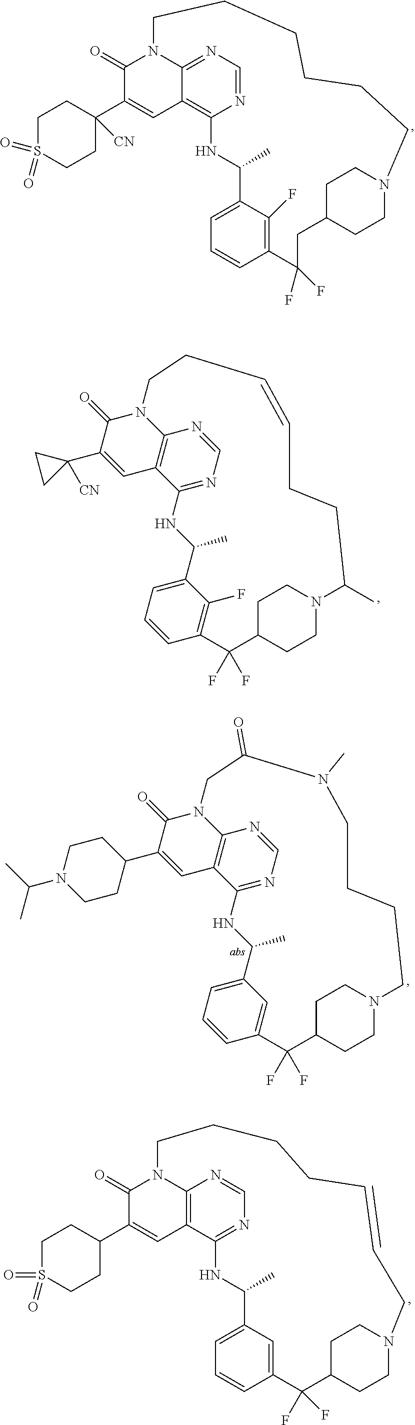 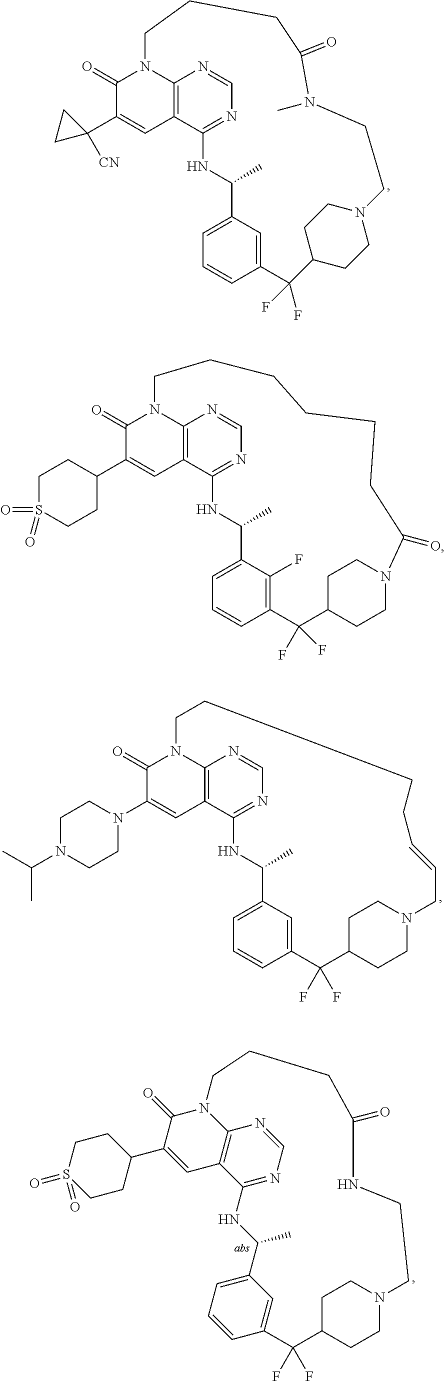 [SMILES: C[C@H]1Nc2ncnc3c2cc(C2(C#N)CC2)c(=O)n3CCCC(=O)N(C)CCN2CCC(CC2)C(F)(F)c2cccc1c2 ; C[C@H]1Nc2ncnc3c2cc(C2CCS(=O)(=O)CC2)c(=O)n3CCCCCCC(=O)N2CCC(CC2)C(F)(F)c2cccc1c2F ; CC(C)N1CCN(c2cc3c4ncnc3n(c2=O)CCCC/C=C/CN2CCC(CC2)C(F)(F)c2cccc(c2)[C@@H](C)N4)CC1 ; C[C@H]1Nc2ncnc3c2cc(C2CCS(=O)(=O)CC2)c(=O)n3CCCC(=O)NCCN2CCC(CC2)C(F)(F)c2cccc1c2] 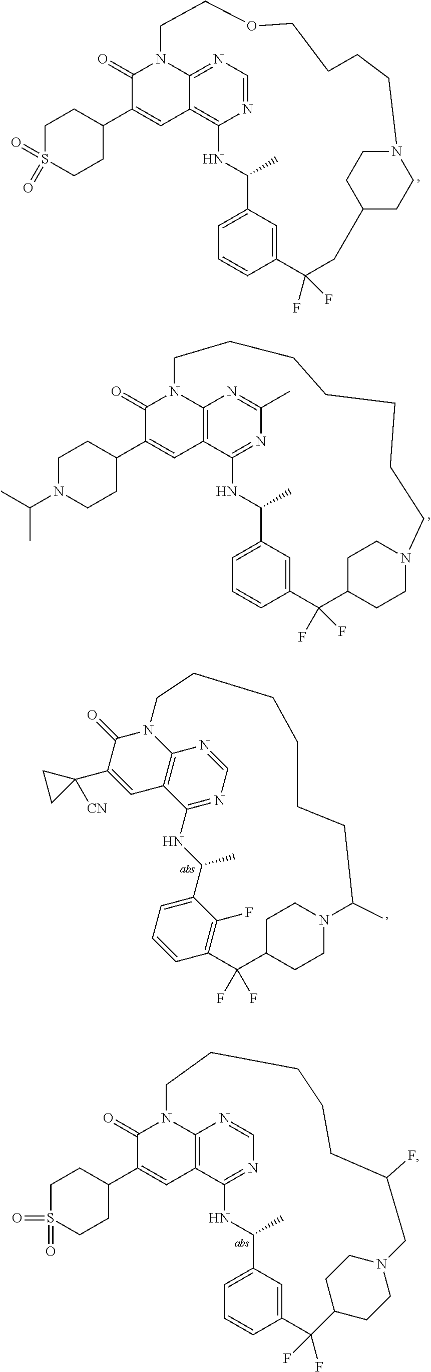 [SMILES: C[C@H]1Nc2ncnc3c2cc(C2CCS(=O)(=O)CC2)c(=O)n3CCOCCCCN2CCC(CC2)CC(F)(F)c2cccc1c2 ; Cc1nc2c3cc(C4CCN(C(C)C)CC4)c(=O)n(c3n1)CCCCCCCN1CCC(CC1)C(F)(F)c1cccc(c1)[C@@H](C)N2 ; CC1CCCCCCn2c(=O)c(C3(C#N)CC3)cc3c(ncnc32)N[C@H](C)c2cccc(c2F)C(F)(F)C2CCN1CC2 ; C[C@H]1Nc2ncnc3c2cc(C2CCS(=O)(=O)CC2)c(=O)n3CCCCCC(F)CN2CCC(CC2)C(F)(F)c2cccc1c2] 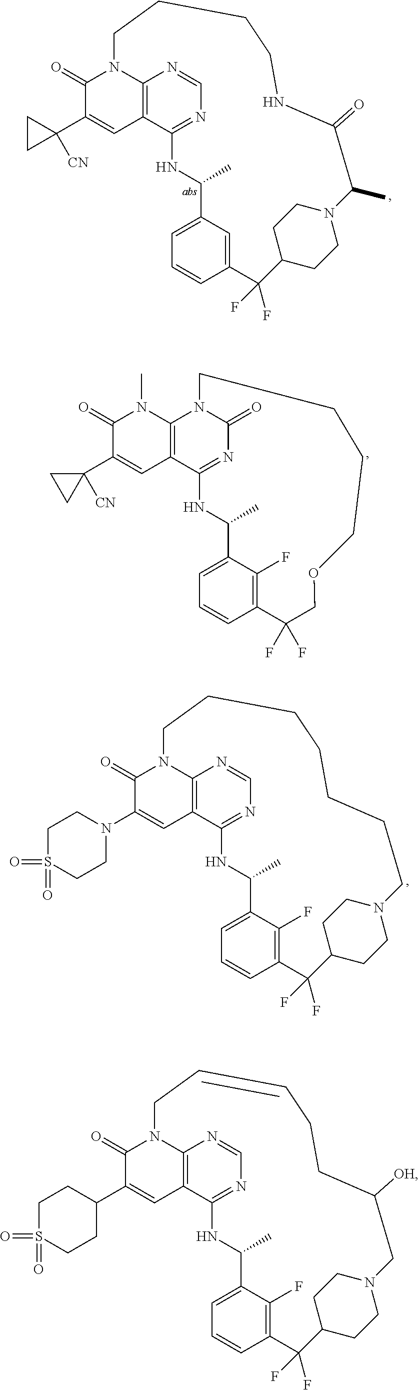 [SMILES: C[C@@H]1C(=O)NCCCCn2c(=O)c(C3(C#N)CC3)cc3c(ncnc32)N[C@H](C)c2cccc(c2)C(F)(F)C2CCN1CC2 ; C[C@H]1Nc2nc(=O)n(c3c2cc(C2(C#N)CC2)c(=O)n3C)CCCCOCC(F)(F)c2cccc1c2F ; C[C@H]1Nc2ncnc3c2cc(N2CCS(=O)(=O)CC2)c(=O)n3CCCCCCCN2CCC(CC2)C(F)(F)c2cccc1c2F ; C[C@H]1Nc2ncnc3c2cc(C2CCS(=O)(=O)CC2)c(=O)n3C/C=C\CCC(O)CN2CCC(CC2)C(F)(F)c2cccc1c2F] 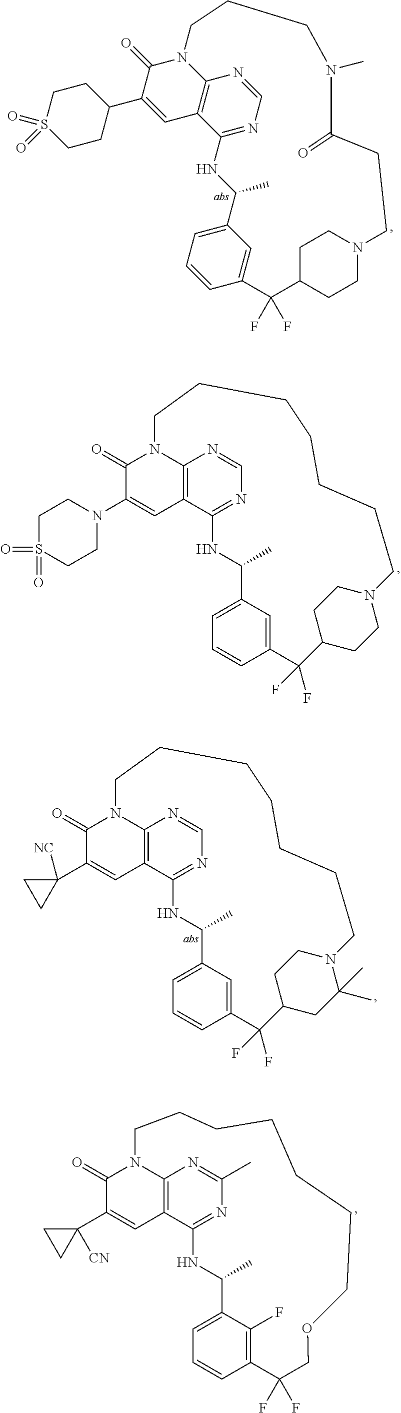 [SMILES: C[C@H]1Nc2ncnc3c2cc(C2CCS(=O)(=O)CC2)c(=O)n3CCCN(C)C(=O)CCN2CCC(CC2)C(F)(F)c2cccc1c2 ; C[C@H]1Nc2ncnc3c2cc(N2CCS(=O)(=O)CC2)c(=O)n3CCCCCCCN2CCC(CC2)C(F)(F)c2cccc1c2 ; C[C@H]1Nc2ncnc3c2cc(C2(C#N)CC2)c(=O)n3CCCCCCCN2CCC(CC2(C)C)C(F)(F)c2cccc1c2 ; Cc1nc2c3cc(C4(C#N)CC4)c(=O)n(c3n1)CCCCCCCOCC(F)(F)c1cccc(c1F)[C@@H](C)N2] 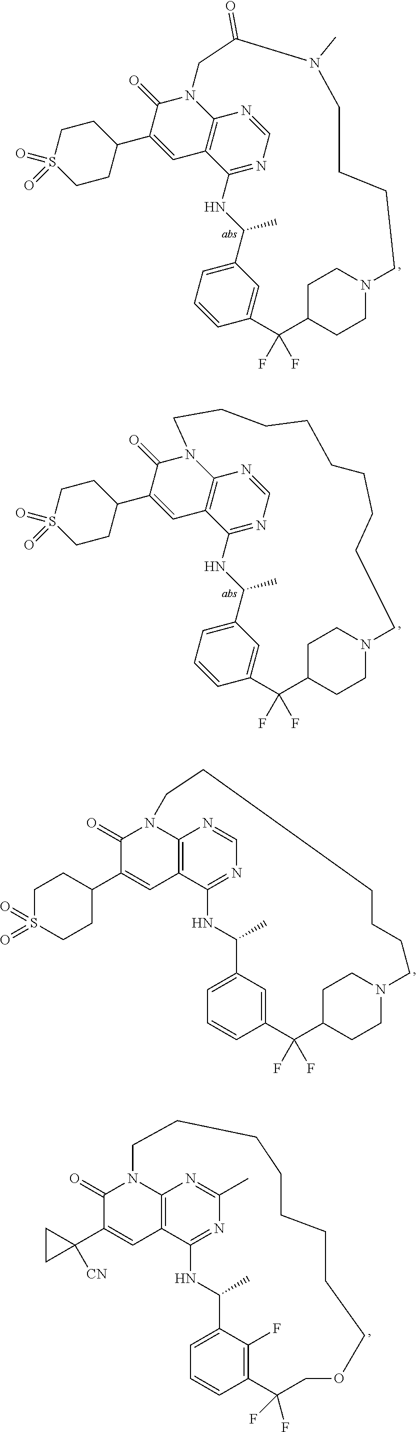 [SMILES: C[C@H]1Nc2ncnc3c2cc(C2CCS(=O)(=O)CC2)c(=O)n3CC(=O)N(C)CCCCN2CCC(CC2)C(F)(F)c2cccc1c2 ; C[C@H]1Nc2ncnc3c2cc(C2CCS(=O)(=O)CC2)c(=O)n3CCCCCCCCCN2CCC(CC2)C(F)(F)c2cccc1c2 ; C[C@H]1Nc2ncnc3c2cc(C2CCS(=O)(=O)CC2)c(=O)n3CCCCCCN2CCC(CC2)C(F)(F)c2cccc1c2 ; Cc1nc2c3cc(C4(C#N)CC4)c(=O)n(c3n1)CCCCCCCCOCC(F)(F)c1cccc(c1F)[C@@H](C)N2] 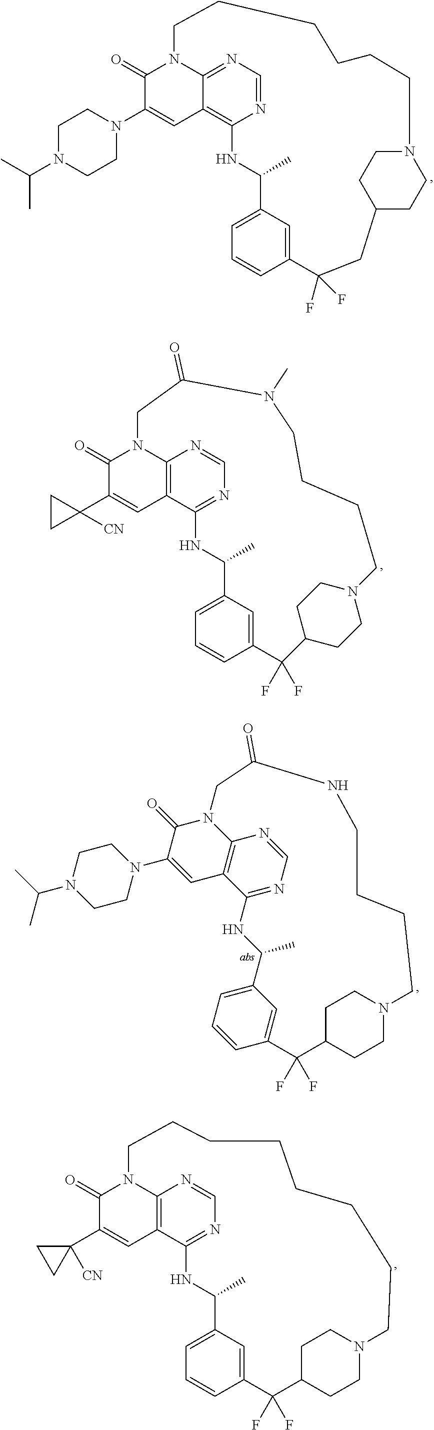 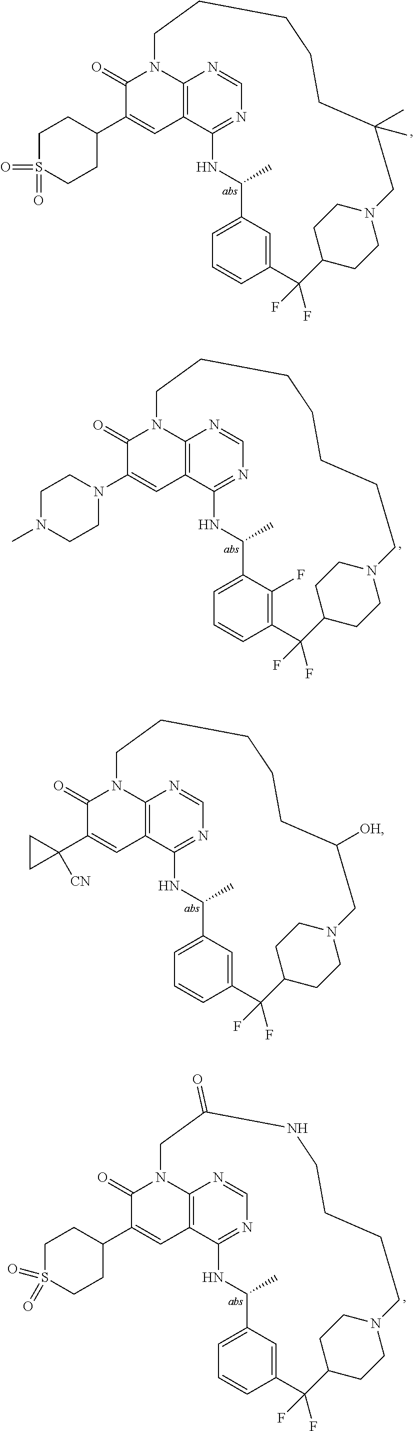 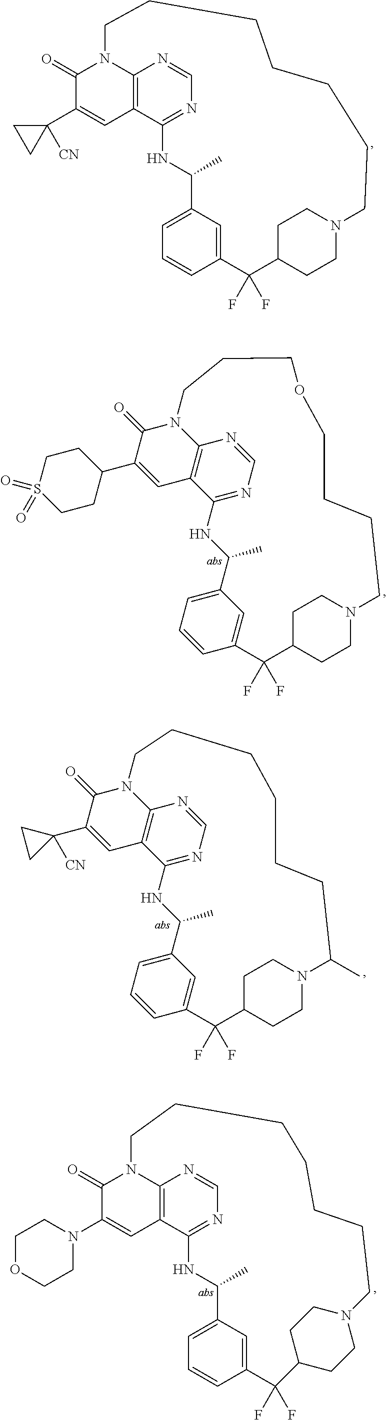 [SMILES: C[C@H]1Nc2ncnc3c2cc(C2(C#N)CC2)c(=O)n3CCCCCCCN2CCC(CC2)C(F)(F)c2cccc1c2 ; C[C@H]1Nc2ncnc3c2cc(C2CCS(=O)(=O)CC2)c(=O)n3CCCOCCCCN2CCC(CC2)C(F)(F)c2cccc1c2 ; CC1CCCCCCn2c(=O)c(C3(C#N)CC3)cc3c(ncnc32)N[C@H](C)c2cccc(c2)C(F)(F)C2CCN1CC2 ; C[C@H]1Nc2ncnc3c2cc(N2CCOCC2)c(=O)n3CCCCCCCN2CCC(CC2)C(F)(F)c2cccc1c2] 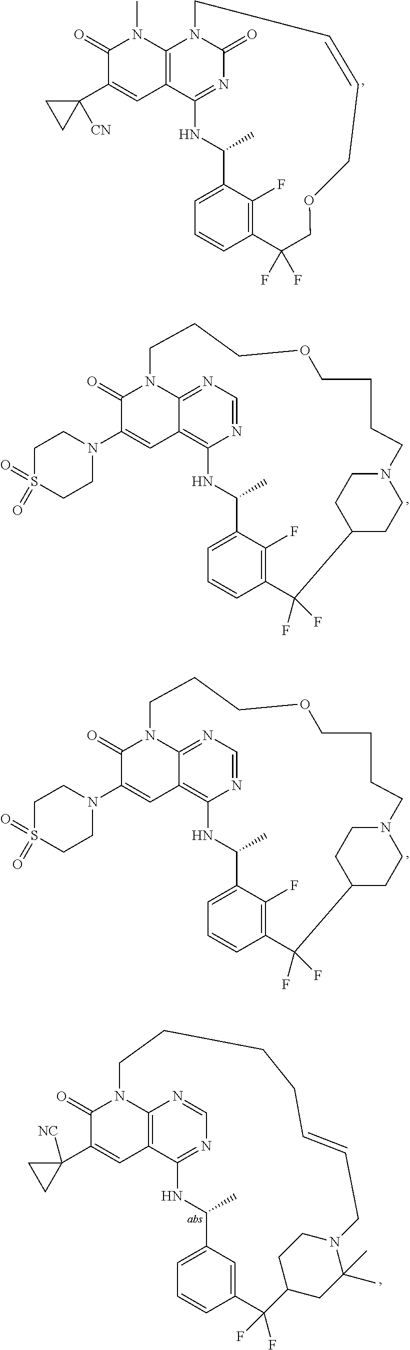 [SMILES: C[C@H]1Nc2nc(=O)n(c3c2cc(C2(C#N)CC2)c(=O)n3C)C/C=C\COCC(F)(F)c2cccc1c2F ; C[C@H]1Nc2ncnc3c2cc(N2CCS(=O)(=O)CC2)c(=O)n3CCCOCCCCN2CCC(CC2)C(F)(F)c2cccc1c2F ; C[C@H]1Nc2ncnc3c2cc(N2CCS(=O)(=O)CC2)c(=O)n3CCCOCCCCN2CCC(CC2)C(F)(F)c2cccc1c2F ; C[C@H]1Nc2ncnc3c2cc(C2(C#N)CC2)c(=O)n3CCCC/C=C/CN2CCC(CC2(C)C)C(F)(F)c2cccc1c2] 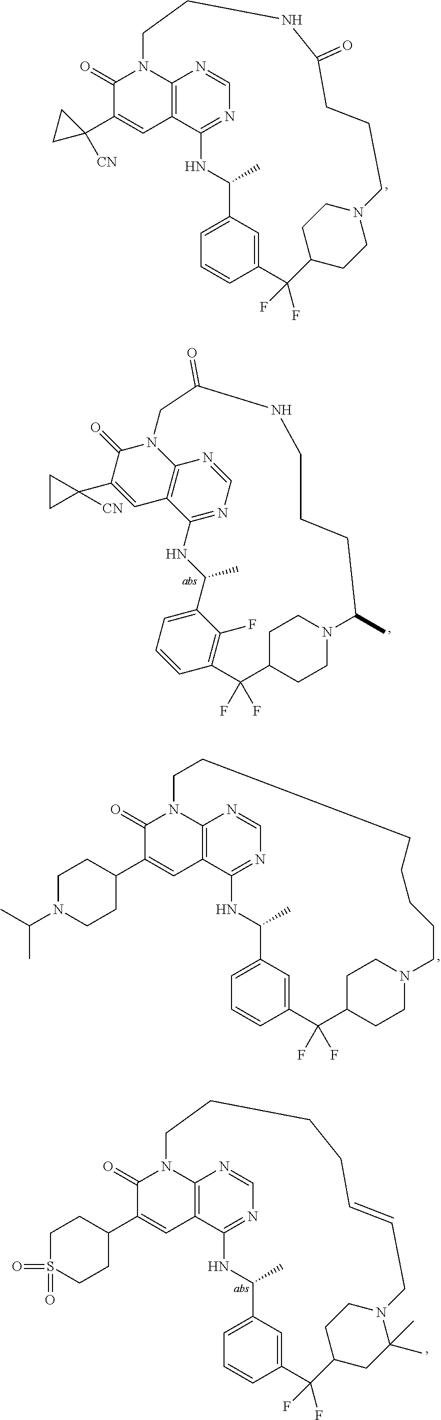 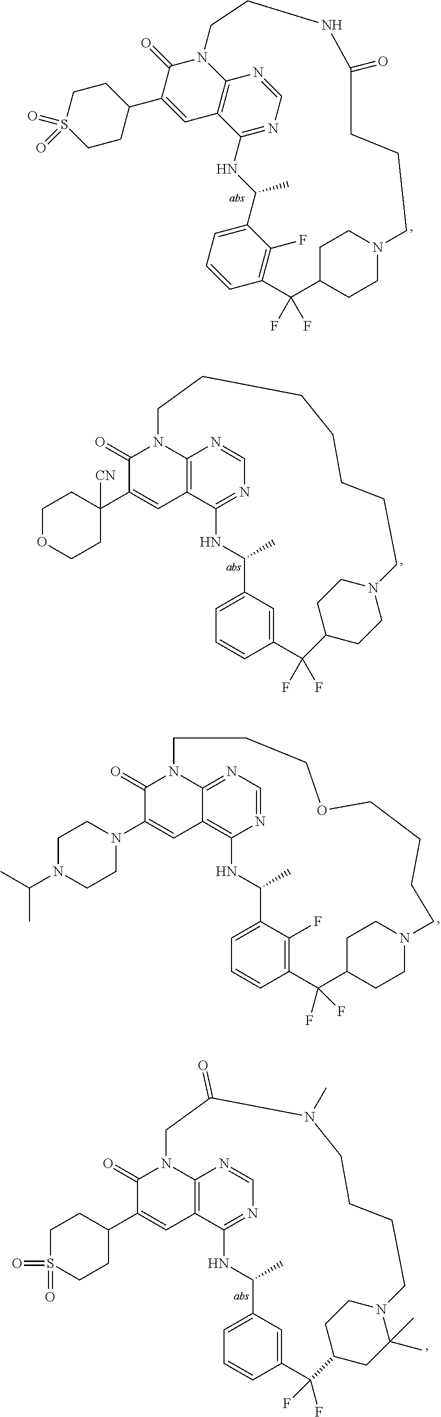 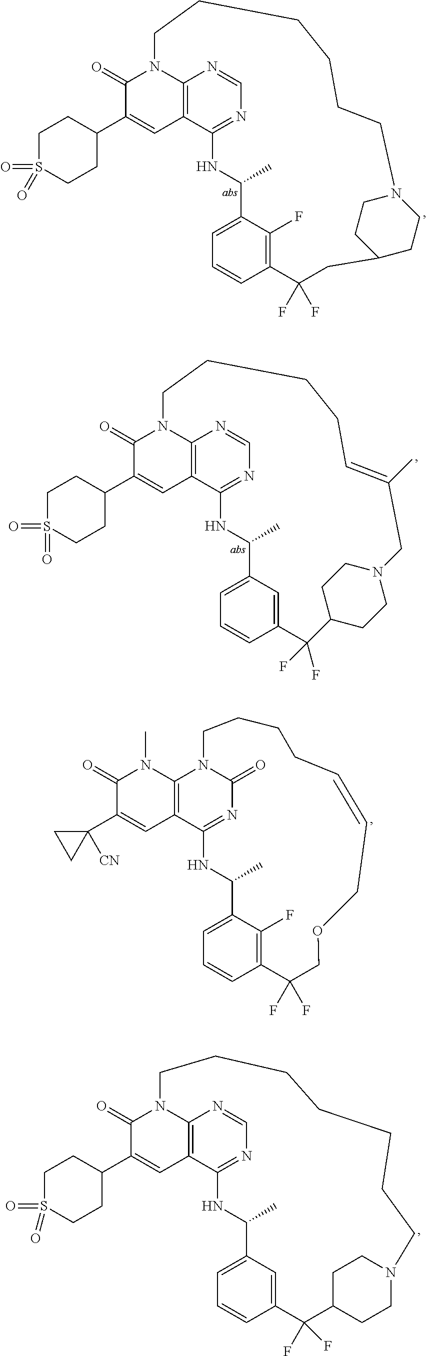 [SMILES: C[C@H]1Nc2ncnc3c2cc(C2CCS(=O)(=O)CC2)c(=O)n3CCCCCCN2CCC(CC2)CC(F)(F)c2cccc1c2F ; C/C1=C\CCCCn2c(=O)c(C3CCS(=O)(=O)CC3)cc3c(ncnc32)N[C@H](C)c2cccc(c2)C(F)(F)C2CCN(CC2)C1 ; C[C@H]1Nc2nc(=O)n(c3c2cc(C2(C#N)CC2)c(=O)n3C)CCCC/C=C\COCC(F)(F)c2cccc1c2F ; C[C@H]1Nc2ncnc3c2cc(C2CCS(=O)(=O)CC2)c(=O)n3CCCCCCCN2CCC(CC2)C(F)(F)c2cccc1c2] 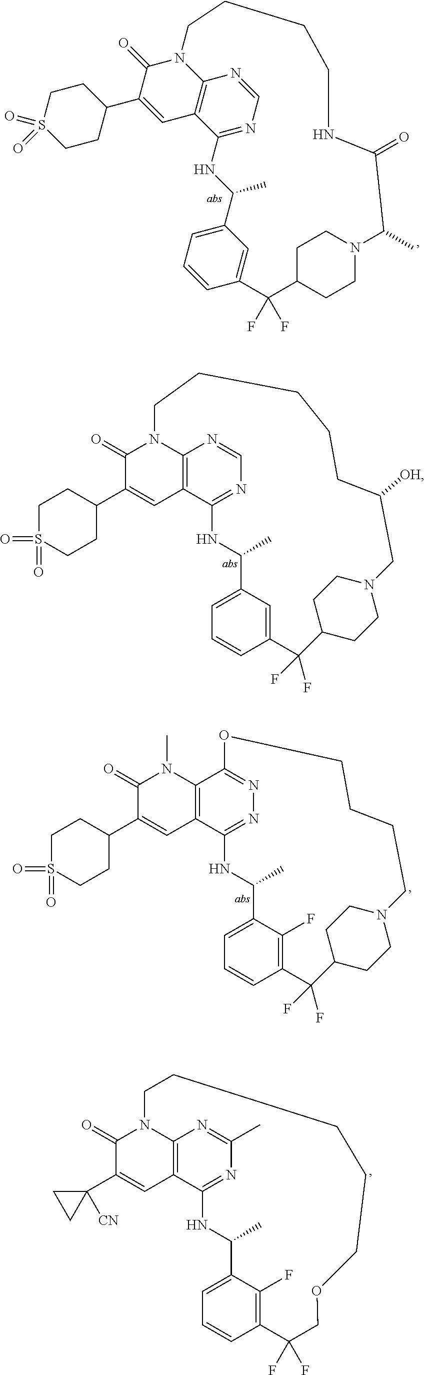 [SMILES: C[C@H]1Nc2ncnc3c2cc(C2CCS(=O)(=O)CC2)c(=O)n3CCCCNC(=O)[C@H](C)N2CCC(CC2)C(F)(F)c2cccc1c2 ; C[C@H]1Nc2ncnc3c2cc(C2CCS(=O)(=O)CC2)c(=O)n3CCCCC[C@H](O)CN2CCC(CC2)C(F)(F)c2cccc1c2 ; C[C@H]1Nc2nnc(c3c2cc(C2CCS(=O)(=O)CC2)c(=O)n3C)OCCCCN2CCC(CC2)C(F)(F)c2cccc1c2F ; Cc1nc2c3cc(C4(C#N)CC4)c(=O)n(c3n1)CCCCCOCC(F)(F)c1cccc(c1F)[C@@H](C)N2] 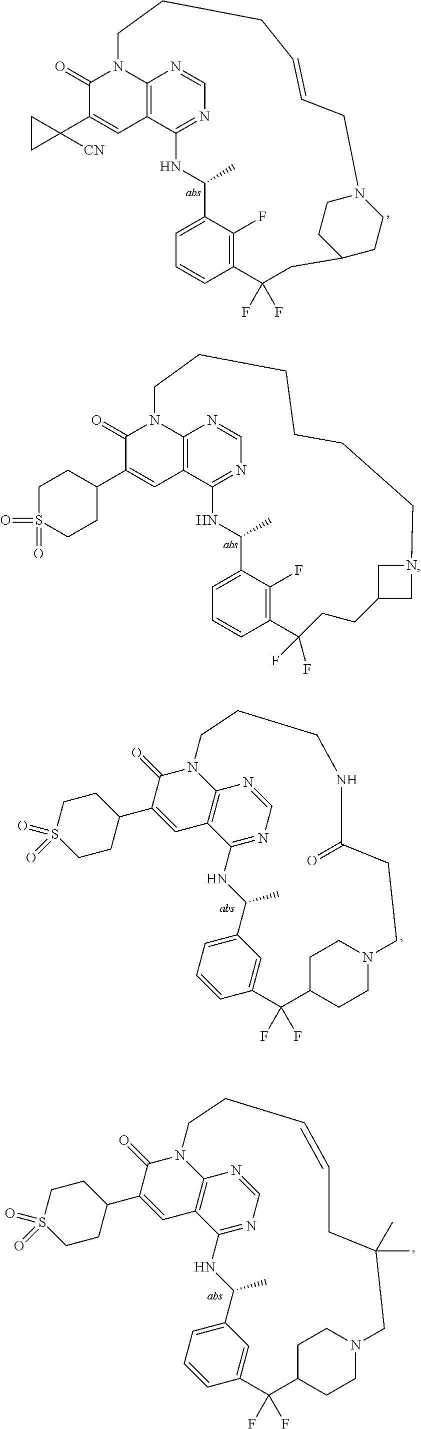 [SMILES: C[C@H]1Nc2ncnc3c2cc(C2(C#N)CC2)c(=O)n3CCC/C=C/CN2CCC(CC2)CC(F)(F)c2cccc1c2F ; C[C@H]1Nc2ncnc3c2cc(C2CCS(=O)(=O)CC2)c(=O)n3CCCCCCN2CC(CCC(F)(F)c3cccc1c3F)C2 ; C[C@H]1Nc2ncnc3c2cc(C2CCS(=O)(=O)CC2)c(=O)n3CCCNC(=O)CCN2CCC(CC2)C(F)(F)c2cccc1c2 ; C[C@H]1Nc2ncnc3c2cc(C2CCS(=O)(=O)CC2)c(=O)n3CC/C=C\CC(C)(C)CN2CCC(CC2)C(F)(F)c2cccc1c2] 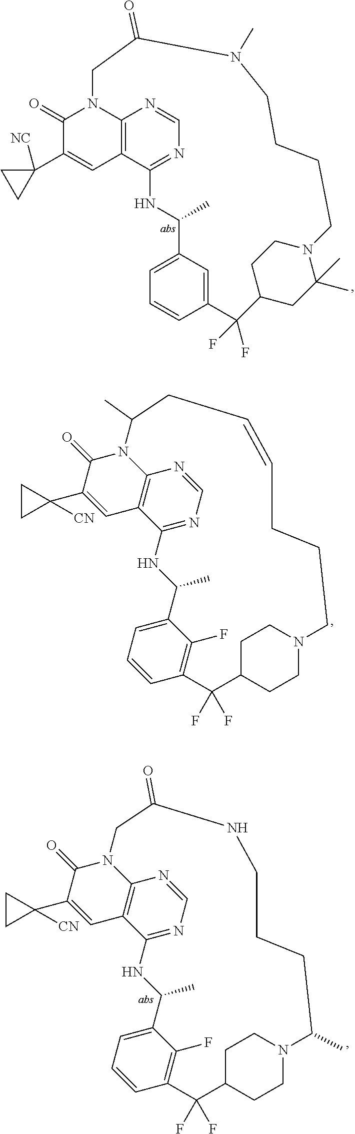 [SMILES: C[C@H]1Nc2ncnc3c2cc(C2(C#N)CC2)c(=O)n3CC(=O)N(C)CCCCN2CCC(CC2(C)C)C(F)(F)c2cccc1c2 ; CC1C/C=C\CCCN2CCC(CC2)C(F)(F)c2cccc(c2F)[C@@H](C)Nc2ncnc3c2cc(C2(C#N)CC2)c(=O)n31 ; C[C@H]1Nc2ncnc3c2cc(C2(C#N)CC2)c(=O)n3CC(=O)NCCC[C@H](C)N2CCC(CC2)C(F)(F)c2cccc1c2F] 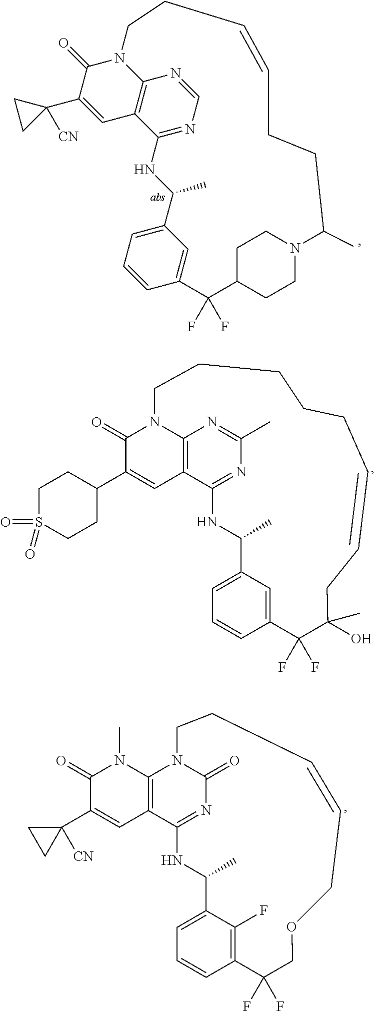 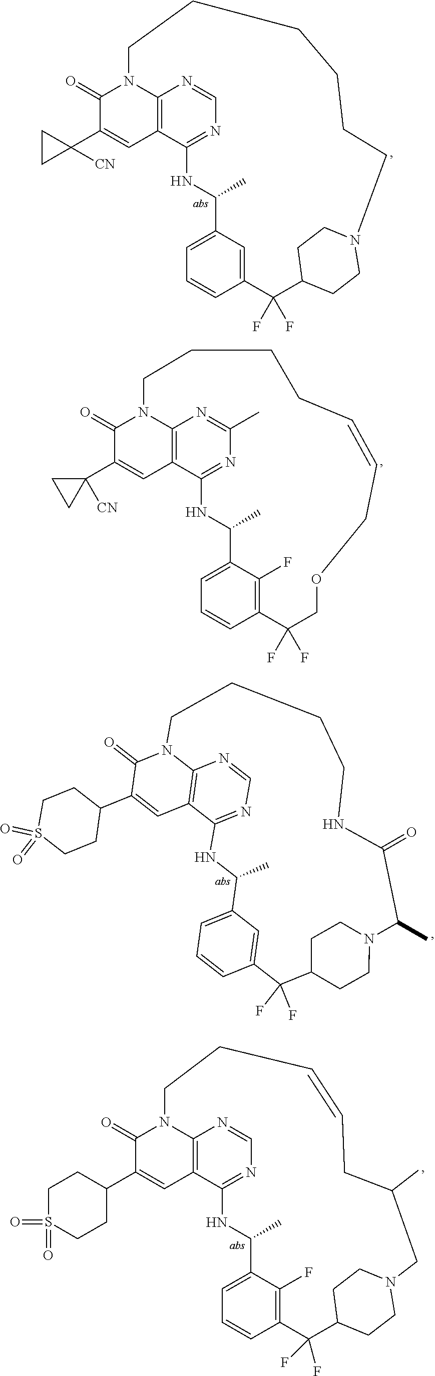 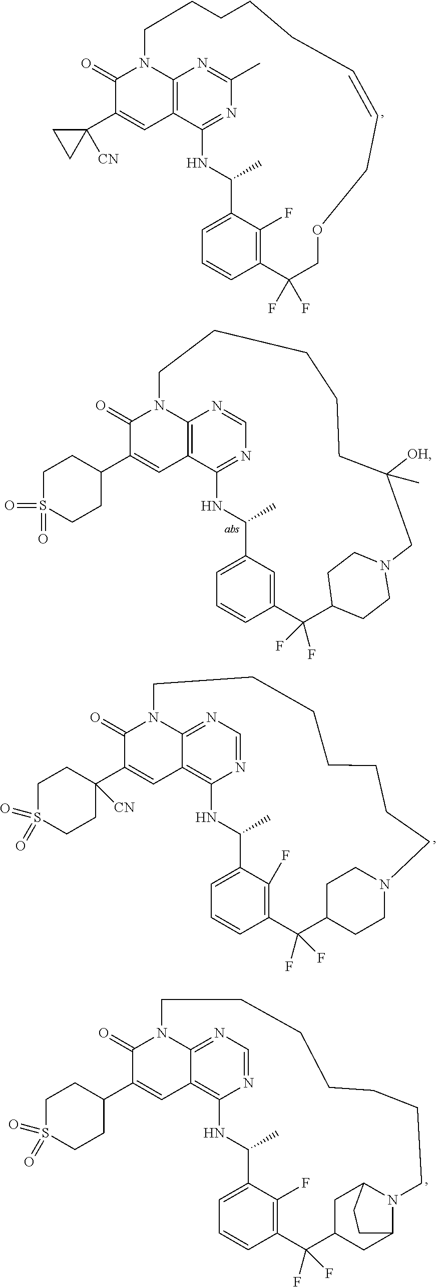 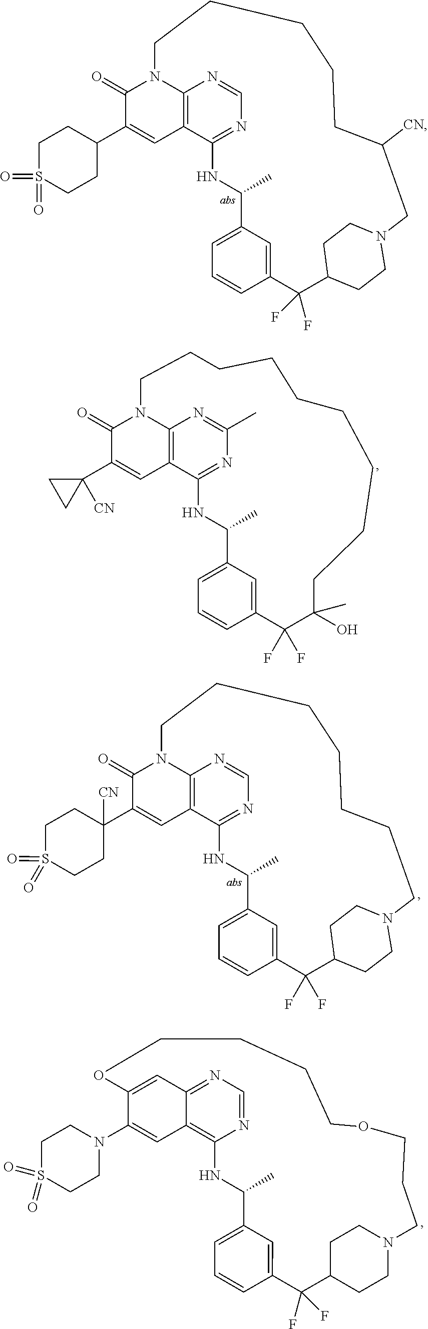 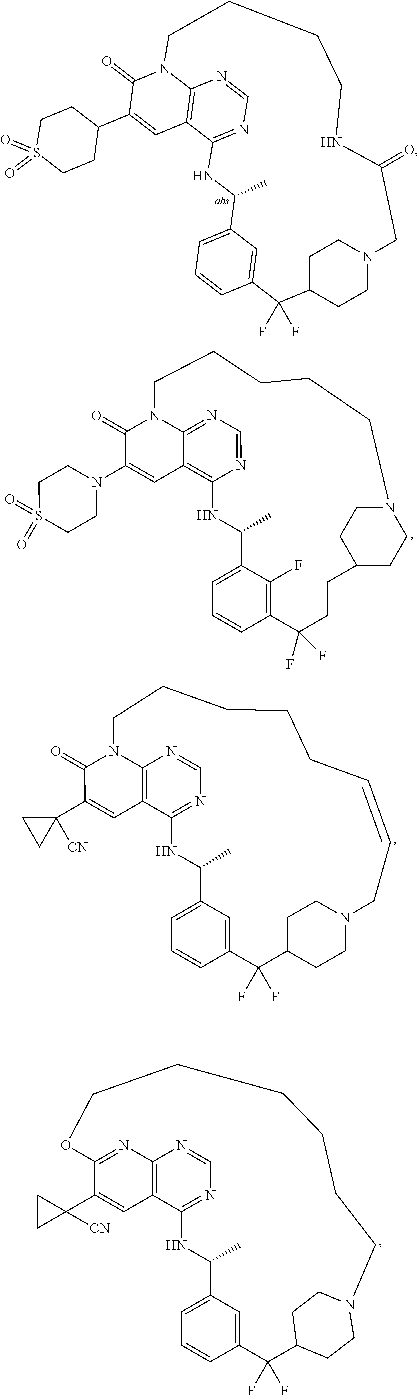 [SMILES: C[C@H]1Nc2ncnc3c2cc(C2CCS(=O)(=O)CC2)c(=O)n3CCCCNC(=O)CN2CCC(CC2)C(F)(F)c2cccc1c2 ; C[C@H]1Nc2ncnc3c2cc(N2CCS(=O)(=O)CC2)c(=O)n3CCCCCN2CCC(CC2)CCC(F)(F)c2cccc1c2F ; C[C@H]1Nc2ncnc3c2cc(C2(C#N)CC2)c(=O)n3CCCCC/C=C\CN2CCC(CC2)C(F)(F)c2cccc1c2 ; C[C@H]1Nc2ncnc3nc(c(C4(C#N)CC4)cc23)OCCCCCCN2CCC(CC2)C(F)(F)c2cccc1c2] 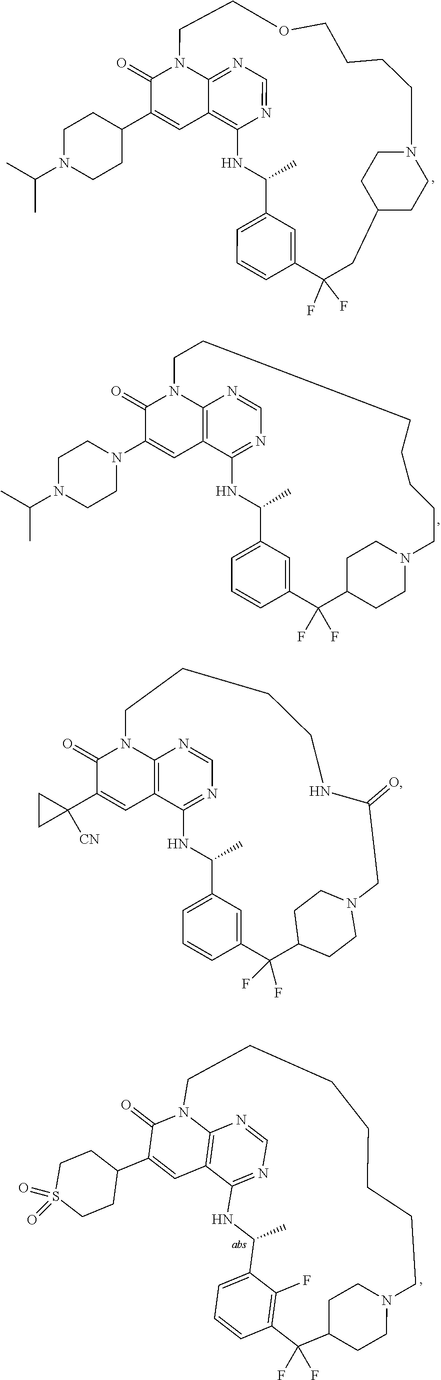 [SMILES: CC(C)N1CCC(c2cc3c4ncnc3n(c2=O)CCOCCCCN2CCC(CC2)CC(F)(F)c2cccc(c2)[C@@H](C)N4)CC1 ; CC(C)N1CCN(c2cc3c4ncnc3n(c2=O)CCCCCCCN2CCC(CC2)C(F)(F)c2cccc(c2)[C@@H](C)N4)CC1 ; C[C@H]1Nc2ncnc3c2cc(C2(C#N)CC2)c(=O)n3CCCCNC(=O)CN2CCC(CC2)C(F)(F)c2cccc1c2 ; C[C@H]1Nc2ncnc3c2cc(C2CCS(=O)(=O)CC2)c(=O)n3CCCCCCCN2CCC(CC2)C(F)(F)c2cccc1c2F] 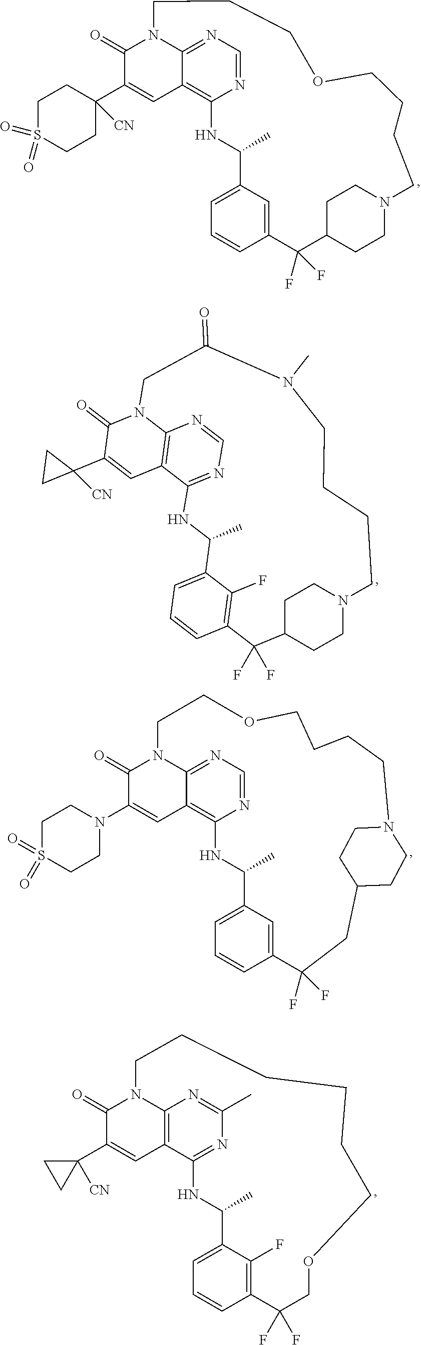 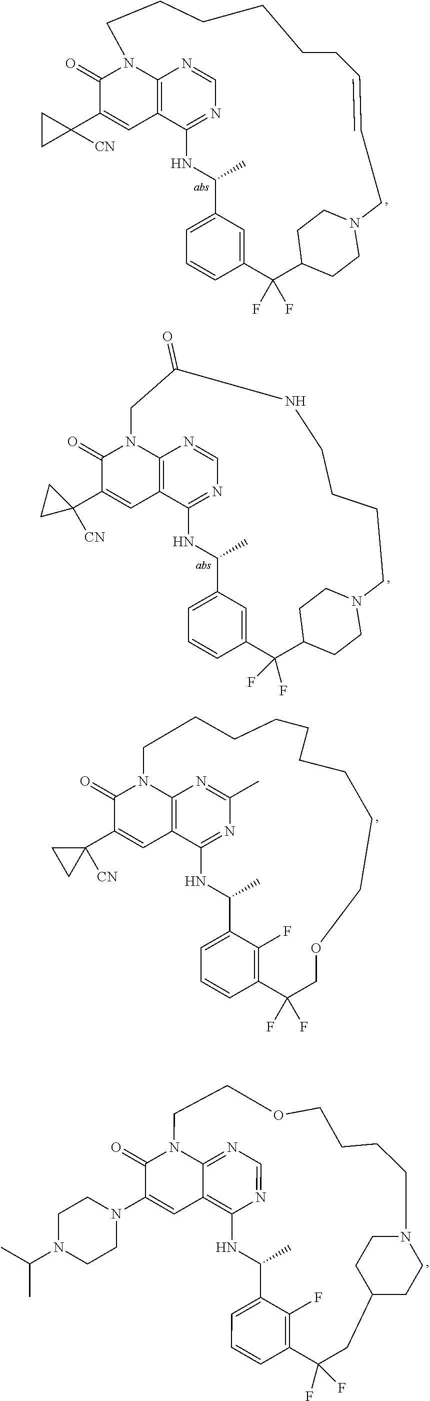 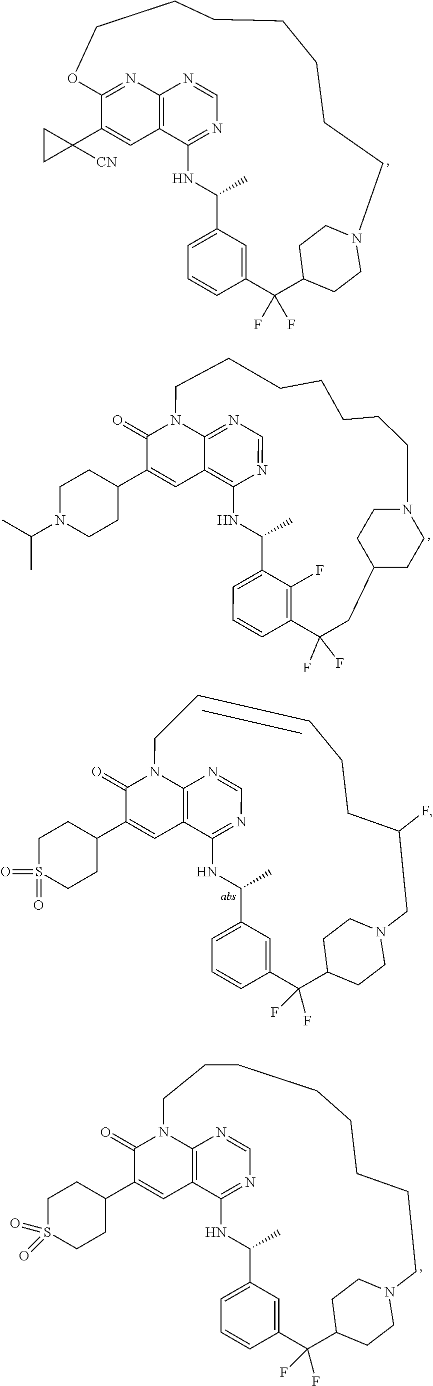 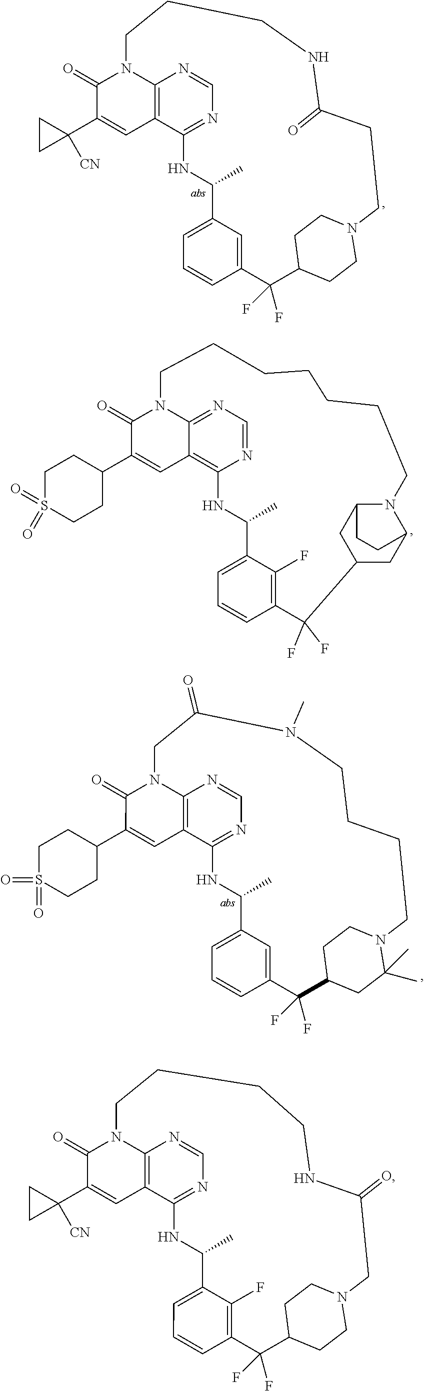 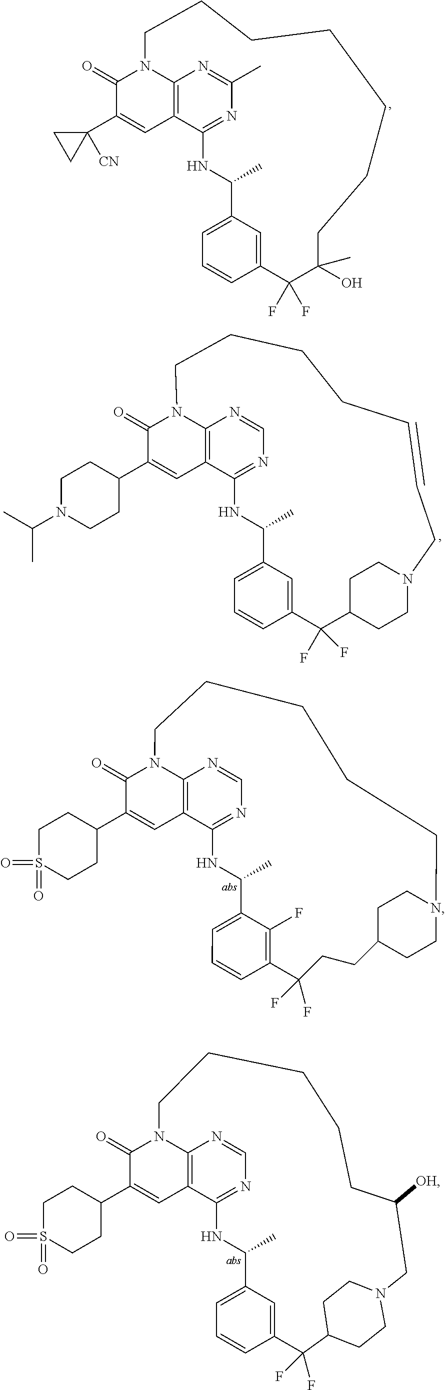 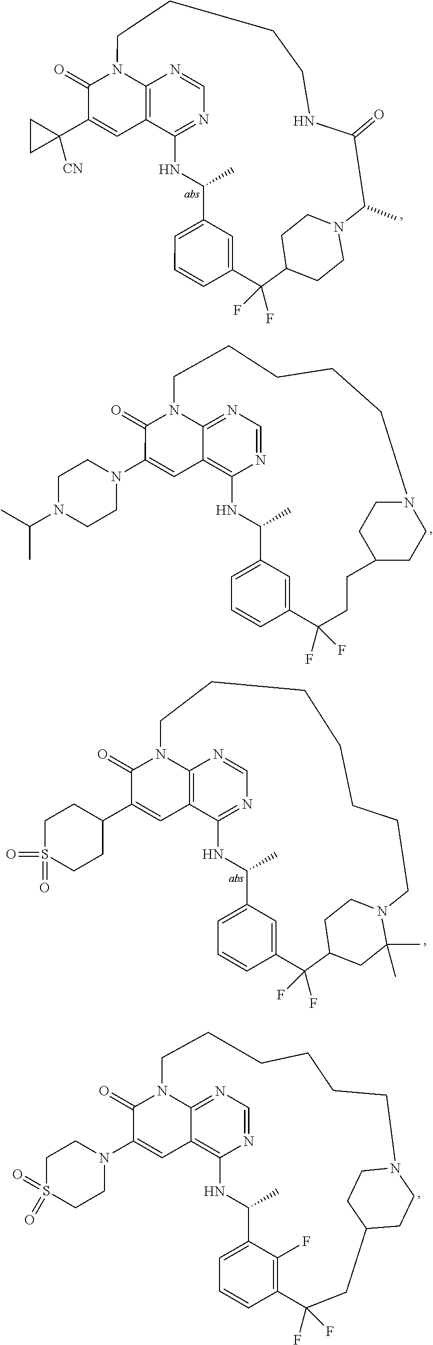 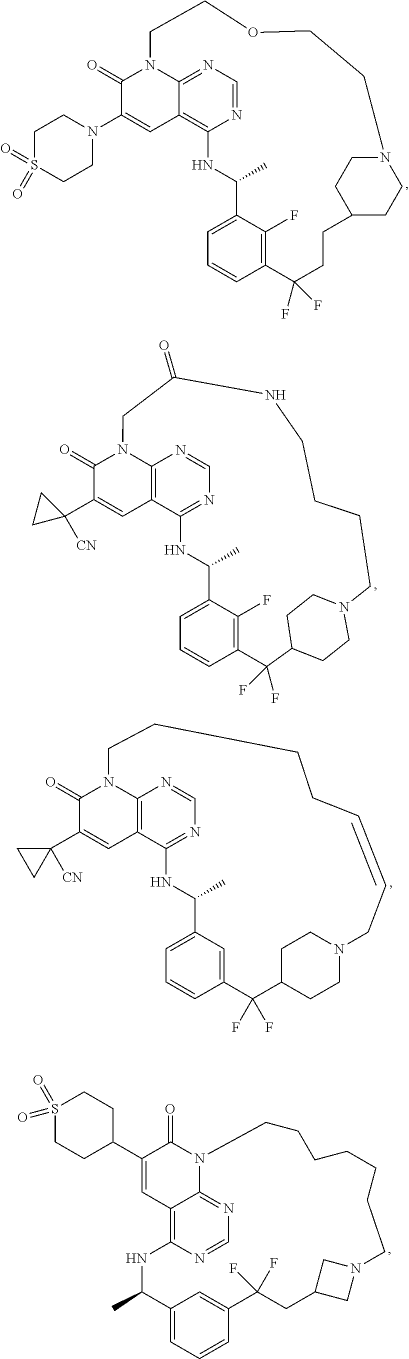 [SMILES: C[C@H]1Nc2ncnc3c2cc(N2CCS(=O)(=O)CC2)c(=O)n3CCOCCN2CCC(CC2)CCC(F)(F)c2cccc1c2F ; C[C@H]1Nc2ncnc3c2cc(C2(C#N)CC2)c(=O)n3CC(=O)NCCCCN2CCC(CC2)C(F)(F)c2cccc1c2F ; C[C@H]1Nc2ncnc3c2cc(C2(C#N)CC2)c(=O)n3CCCC/C=C\CN2CCC(CC2)C(F)(F)c2cccc1c2 ; C[C@H]1Nc2ncnc3c2cc(C2CCS(=O)(=O)CC2)c(=O)n3CCCCCCCN2CC(C2)CC(F)(F)c2cccc1c2] 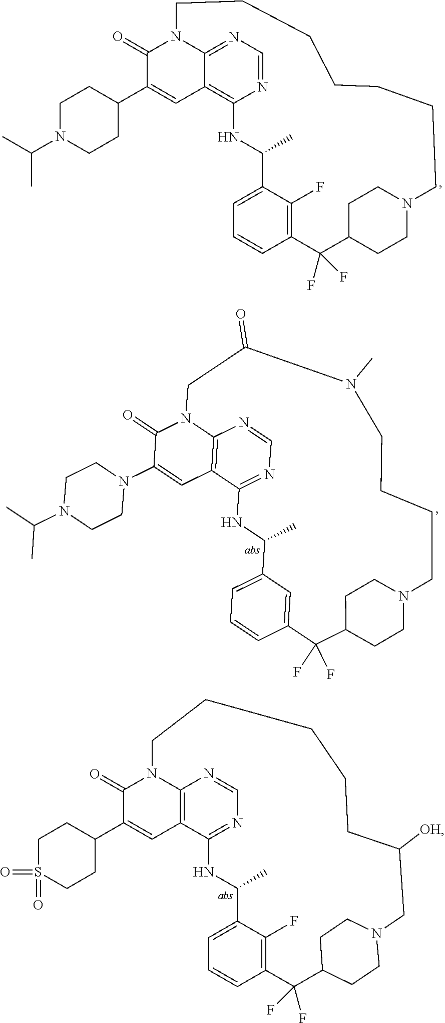 [SMILES: CC(C)N1CCC(c2cc3c4ncnc3n(c2=O)CCCCCCCN2CCC(CC2)C(F)(F)c2cccc(c2F)[C@@H](C)N4)CC1 ; CC(C)N1CCN(c2cc3c4ncnc3n(c2=O)CC(=O)N(C)CCCCN2CCC(CC2)C(F)(F)c2cccc(c2)[C@@H](C)N4)CC1 ; C[C@H]1Nc2ncnc3c2cc(C2CCS(=O)(=O)CC2)c(=O)n3CCCCCC(O)CN2CCC(CC2)C(F)(F)c2cccc1c2F] 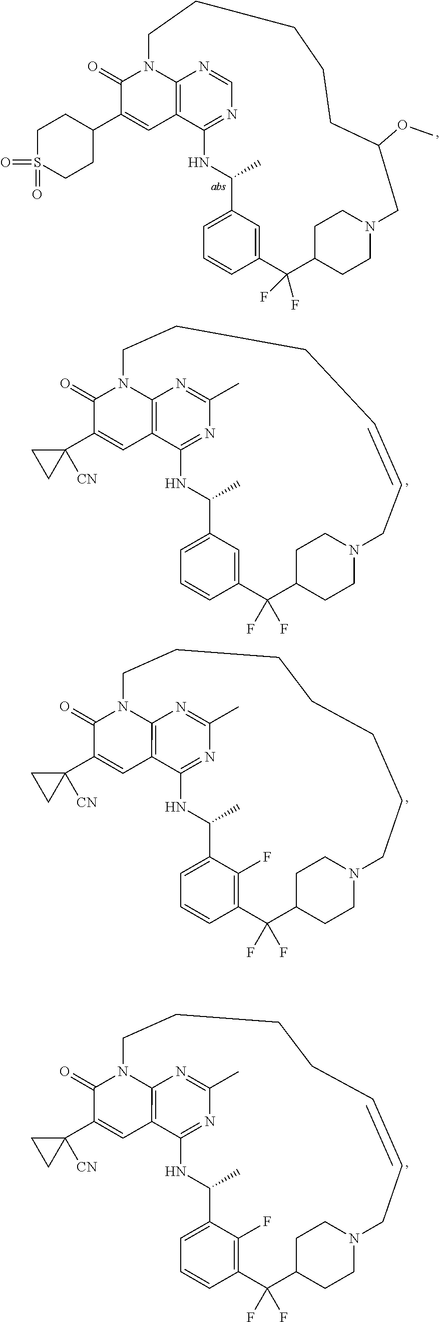 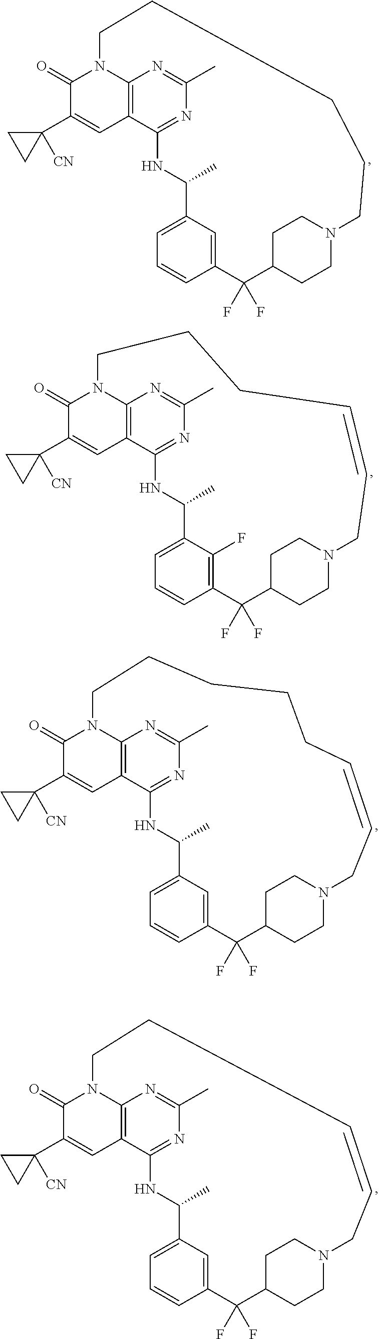 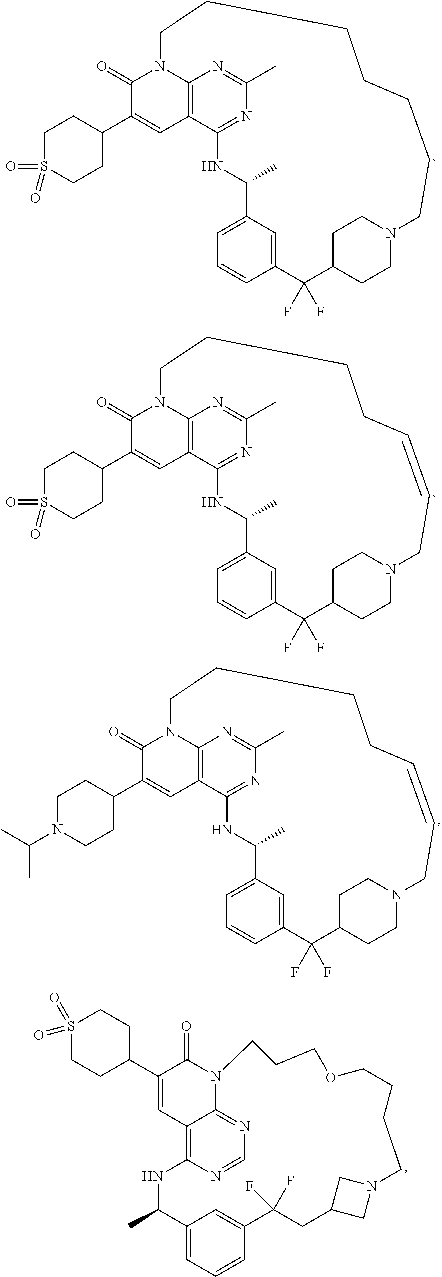 [SMILES: Cc1nc2c3cc(C4CCS(=O)(=O)CC4)c(=O)n(c3n1)CCCCCCCN1CCC(CC1)C(F)(F)c1cccc(c1)[C@@H](C)N2 ; Cc1nc2c3cc(C4CCS(=O)(=O)CC4)c(=O)n(c3n1)CCCC/C=C\CN1CCC(CC1)C(F)(F)c1cccc(c1)[C@@H](C)N2 ; Cc1nc2c3cc(C4CCN(C(C)C)CC4)c(=O)n(c3n1)CCCC/C=C\CN1CCC(CC1)C(F)(F)c1cccc(c1)[C@@H](C)N2 ; C[C@H]1Nc2ncnc3c2cc(C2CCS(=O)(=O)CC2)c(=O)n3CCCOCCCCN2CC(C2)CC(F)(F)c2cccc1c2] 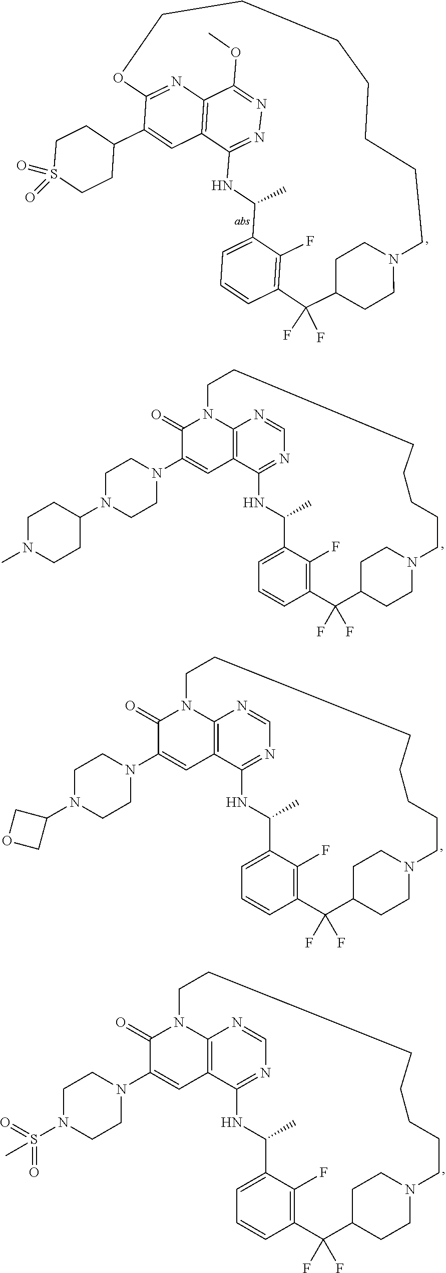 [SMILES: COc1nnc2c3cc(C4CCS(=O)(=O)CC4)c(nc13)OCCCCCCCN1CCC(CC1)C(F)(F)c1cccc(c1F)[C@@H](C)N2 ; C[C@H]1Nc2ncnc3c2cc(N2CCN(C4CCN(C)CC4)CC2)c(=O)n3CCCCCCCN2CCC(CC2)C(F)(F)c2cccc1c2F ; C[C@H]1Nc2ncnc3c2cc(N2CCN(C4COC4)CC2)c(=O)n3CCCCCCCN2CCC(CC2)C(F)(F)c2cccc1c2F ; C[C@H]1Nc2ncnc3c2cc(N2CCN(S(C)(=O)=O)CC2)c(=O)n3CCCCCCCN2CCC(CC2)C(F)(F)c2cccc1c2F] 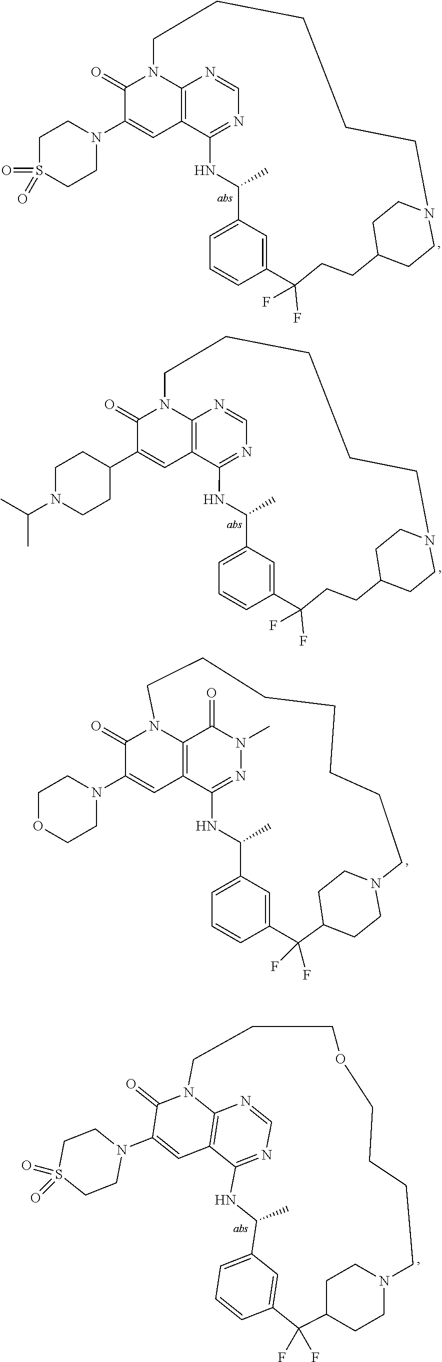 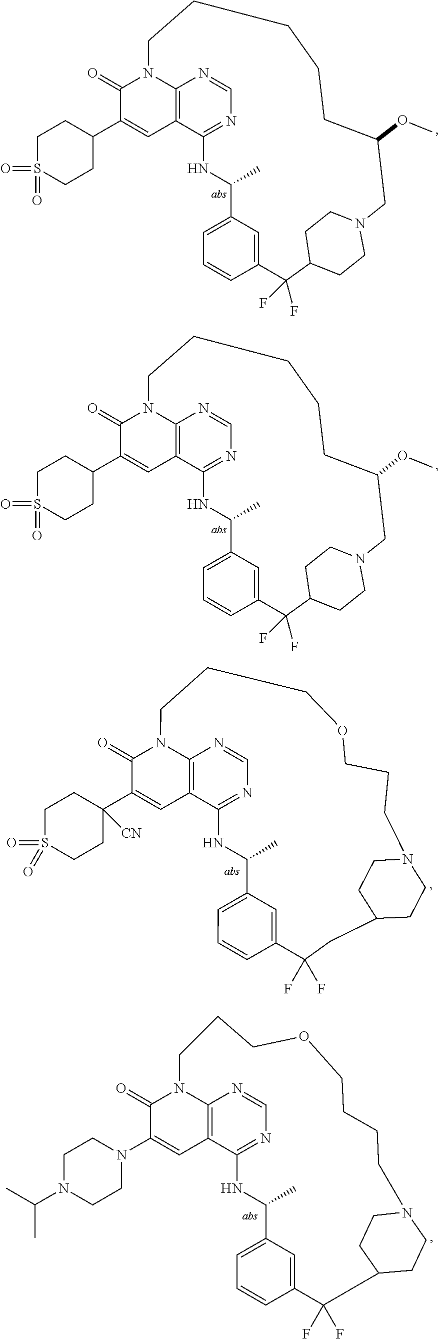 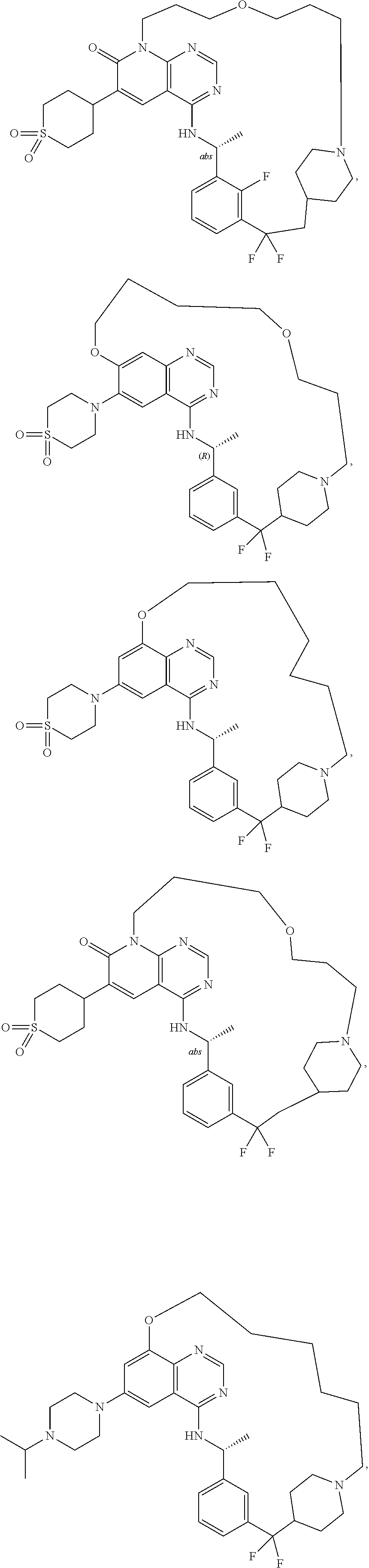 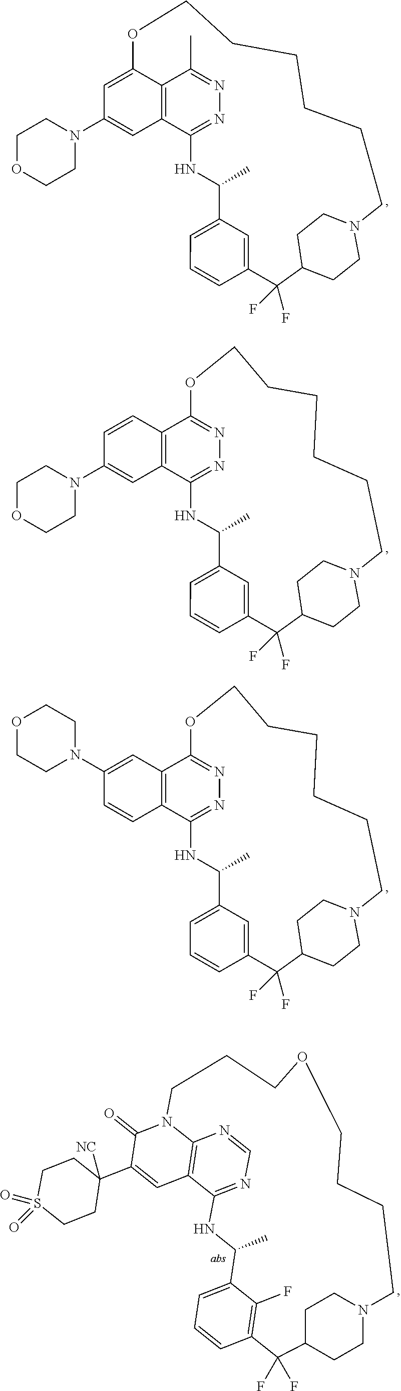 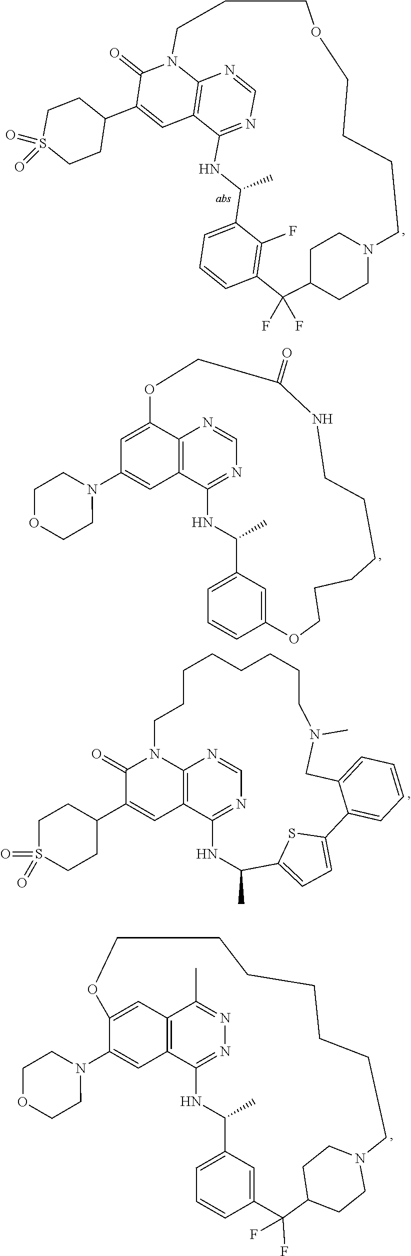 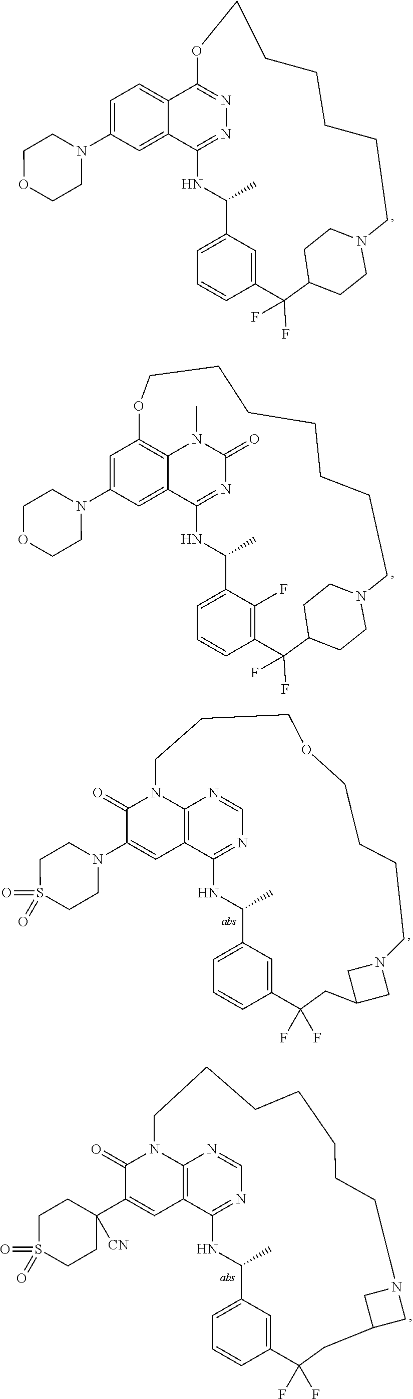 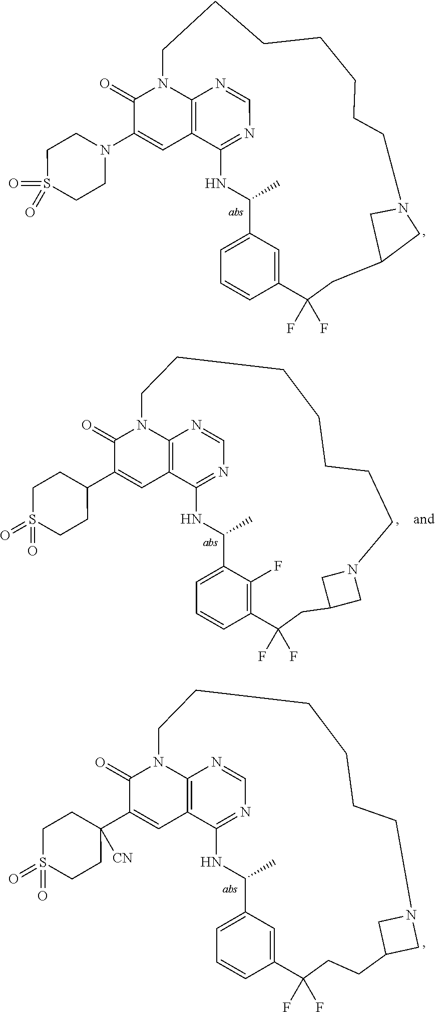
or a pharmaceutically acceptable salt or solvate thereof.

94. The method of claim 37, wherein the compound of Formula (I-1) is 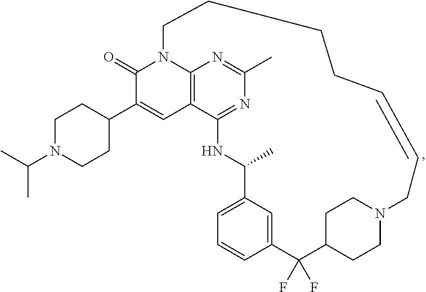 [SMILES: Cc1nc2c3cc(C4CCN(C(C)C)CC4)c(=O)n(c3n1)CCCC/C=C\CN1CCC(CC1)C(F)(F)c1cccc(c1)[C@@H](C)N2]
or a pharmaceutically acceptable salt or solvate thereof.

95. The method of claim 37, wherein the compound of Formula (I-1) is 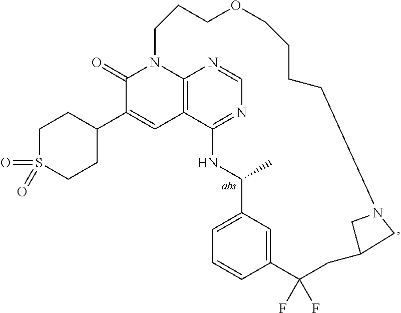 [SMILES: C[C@H]1Nc2ncnc3c2cc(C2CCS(=O)(=O)CC2)c(=O)n3CCCOCCCCN2CC(C2)CC(F)(F)c2cccc1c2]
or a pharmaceutically acceptable salt or solvate thereof.
